(19) United States
(12) Patent Application Publication
(10) Pub. No.: US 2025/0115807 A1
(43) Pub. Date: Apr. 10, 2025

(54) ARYLAMINE COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Xianbin XU, Xi'an (CN); Lei YANG, Xi'an (CN)

(21) Appl. No.: 18/725,896

(22) PCT Filed: May 24, 2023
(86) PCT No.: PCT/CN2023/096137

§ 371 Date: Jul. 1, 2024

(30) Foreign Application Priority Data
Aug. 22, 2022 (CN) 202211006825.5

(51) Int. Cl. C09K 11/06; H10K 50/11; H10K 85/60

(52) U.S. Cl. CPC C09K 11/06; H10K 50/11; H10K 85/633; C09K 2211/1011

(57) ABSTRACT

The disclosure relates to the technical field of organic electroluminescent materials, and provides an arylamine compound, and an organic electroluminescent device and an electronic apparatus thereof. The arylamine compound of the disclosure includes a [5]helicene group, and when the compound is used as a host material of an organic electroluminescent device, the efficiency and the service life of the device can be significantly improved.

### CROSS REFERENCE TO RELATED APPLICATIONS

This application is a National Stage Entry of International Application No. PCT/CN2023/096137, filed May 24, 2023, and claims priority to Chinese Patent Application No. CN202211006825.5, filed on Aug. 22, 2022, the disclosures of which are incorporated herein by reference in their entireties.

### FIELD OF THE INVENTION

The disclosure relates to the technical field of organic electroluminescent materials, in particular to an arylamine compound, and an organic electroluminescent device and an electronic apparatus thereof.

### BACKGROUND OF THE INVENTION

With the development of electronic technology and the progress of material science, the application range of electronic components for realizing electroluminescence or photoelectric conversion is wider and wider. An organic electroluminescent device (OLED) typically includes a cathode and an anode which are oppositely disposed, and a functional layer disposed between the cathode and the anode. The functional layer is composed of a plurality of organic film layers or inorganic film layers, and generally includes an organic electroluminescent layer, a hole transport layer, an electron transport layer, and the like. When a voltage is applied to the cathode and the anode, an electric field is generated between the cathode and the anode, electrons on a cathode side move towards an electroluminescent layer and holes on an anode side also move towards the electroluminescent layer under the action of the electric field, the electrons and the holes are combined in the electroluminescent layer to form excitons, the excitons are in an excited state and release energy outwards, and then the electroluminescent layer emits light outwards.

In the existing organic electroluminescent device, the most major problems are reflected in service life and efficiency, as an area of a display becomes larger, a driving voltage also increases, and the luminous efficiency and the current efficiency also need to be improved, and thus, it is necessary to continue to develop new materials to further improve the performance of the organic electroluminescent device.

### SUMMARY OF THE INVENTION

In view of the above problems existing in the prior art, an object of the disclosure is to provide an arylamine compound, and an organic electroluminescent device and an electronic apparatus thereof. When the arylamine compound is used in an organic electroluminescent device, the performance of the device can be improved.

According to a first aspect of the disclosure, provided is an arylamine compound, having a structure represented by a formula 1:

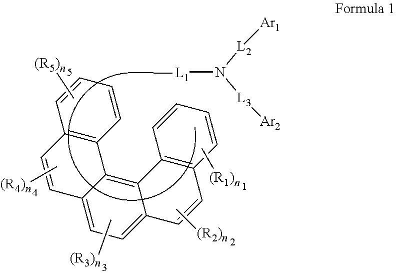

where L₁, L₂ and L₃ are the same or different, and are each independently selected from a single bond, substituted or unsubstituted arylene with 6 to 30 carbon atoms, and substituted or unsubstituted heteroarylene with 3 to 30 carbon atoms;
Ar₁ and Ar₂ are the same or different, and are each independently selected from substituted or unsubstituted aryl with 6 to 30 carbon atoms, and substituted or unsubstituted heteroaryl with 3 to 30 carbon atoms;
R₁, R₂, R₃, R₄ and R₅ are the same or different, and are each independently selected from hydrogen, deuterium, cyano, aryl with 6 to 20 carbon atoms, heteroaryl with 3 to 20 carbon atoms, alkyl with 1 to 10 carbon atoms, alkoxy with 1 to 10 carbon atoms, haloalkyl with 1 to 10 carbon atoms, trialkylsilyl with 3 to 12 carbon atoms, triphenylsilyl, cycloalkyl with 3 to 10 carbon atoms, and deuteroalkyl with 1 to 10 carbon atoms;
n₁ and n₅ are each independently selected from 0, 1, 2, 3 or 4; n₂, n₃ and n₄ are each independently selected from 0, 1 or 2; and
Substituent(s) in L₁, L₂, L₃, Ar₁ and Ar₂ are the same or different, and are each independently selected from deuterium, fluorine, cyano, aryl with 6 to 20 carbon atoms, heteroaryl with 3 to 20 carbon atoms, alkyl with 1 to 10 carbon atoms, alkoxy with 1 to 10 carbon atoms, haloalkyl with 1 to 10 carbon atoms, trialkylsilyl with 3 to 12 carbon atoms, triphenylsilyl, cycloalkyl with 3 to 10 carbon atoms, and deuteroalkyl with 1 to 10 carbon atoms; and optionally, any two adjacent substituents in Ar₁ and Ar₂ form a saturated or unsaturated 3- to 15-membered ring.

According to a second aspect of the disclosure, provided is an organic electroluminescent device, including an anode and a cathode which are oppositely disposed, and a functional layer disposed between the anode and the cathode; where the functional layer includes the arylamine compound described above.

According to a third aspect of the disclosure, provided is an electronic apparatus, including the organic electroluminescent device in the second aspect.

The compound of the disclosure is of a triarylamine structure formed with [5]helicene as a parent nucleus. On the one hand, [5]helicene has a larger conjugation plane and rigidity, and arylamines have excellent hole transport properties, and after [5]helicene is linked with arylamines, hole mobility of compounds can be further improved. On the other hand, the first ring and the fifth benzene ring at the end of [5]helicene

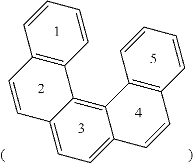 [SMILES: c1ccc2c(c1)ccc1ccc3ccc4ccccc4c3c12]

are in different planes due to the steric hindrance effect of hydrogen atoms, thus forming spatial planes with a plurality of different included angles within the molecule, which can effectively inhibit stacking between molecules, and improve the film-forming properties of the compounds. When the compound of the disclosure is used as a hole transport type host material in a hybrid type host material, the balance of carriers in an electroluminescent layer can be improved, the carrier utilization rate can be improved, and a recombination region of carriers can be broadened, thus significantly improving the efficiency and service life of devices.

### BRIEF DESCRIPTION OF DRAWINGS

The accompanying drawings are used to provide a further understanding of the disclosure and constitute a part of the description, and are used to explain the disclosure together with the following specific embodiments, but do not constitute limitations on the disclosure.

FIG. 1 is a structural schematic diagram of an organic electroluminescent device according to an embodiment of the disclosure.

FIG. 2 is a structural schematic diagram of an electronic apparatus according to an embodiment of the disclosure.

Reference signs

| | | | |
|---|---|---|---|
| 100, anode | 200, cathode | 300, functional | 310, hole |
| | | layer | injection |
| | | | layer |
| 321, hole | 322, hole | 330, organic | 340, electron |
| transport | adjustment | electroluminescent | transport |
| layer | layer | layer | layer |
| 350, electron | 400, electronic | | |
| injection layer | apparatus | | |

### DETAILED DESCRIPTION

Exemplary embodiments will now be described more fully with reference to the accompanying drawings. However, the exemplary embodiments can be implemented in a variety of forms, and should not be understood as a limitation to the instances set forth here; and on the contrary, these embodiments are provided such that the disclosure will be more comprehensive and complete, and the concepts of the examples are comprehensively conveyed to those skilled in the art. The described features, structures, or characteristics may be incorporated in one or more embodiments in any suitable manner. In the following description, many specific details are provided to give a sufficient understanding of the embodiments of the disclosure.

In a first aspect, the disclosure provides an arylamine compound, having a structure represented by a formula 1:

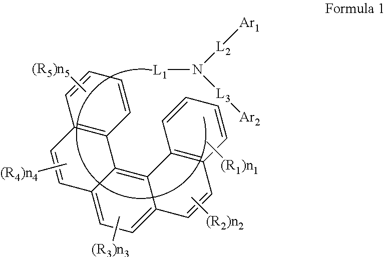

where L₁, L₂ and L₃ are the same or different, and are each independently selected from a single bond, substituted or unsubstituted arylene with 6 to 30 carbon atoms, and substituted or unsubstituted heteroarylene with 3 to 30 carbon atoms;
Ar₁ and Ar₂ are the same or different, and are each independently selected from substituted or unsubstituted aryl with 6 to 30 carbon atoms, and substituted or unsubstituted heteroaryl with 3 to 30 carbon atoms;
R₁, R₂, R₃, R₄ and R₅ are the same or different, and are each independently selected from hydrogen, deuterium, cyano, aryl with 6 to 20 carbon atoms, heteroaryl with 3 to 20 carbon atoms, alkyl with 1 to 10 carbon atoms, alkoxy with 1 to 10 carbon atoms, haloalkyl with 1 to 10 carbon atoms, trialkylsilyl with 3 to 12 carbon atoms, triphenylsilyl, cycloalkyl with 3 to 10 carbon atoms, and deuteroalkyl with 1 to 10 carbon atoms;
n₁, n₂, n₃, n₄ and n₅ independently represent the number of substituent(s) R₁, R₂, R₃, R₄ and R₅, respectively;
n₁ and n₅ are each independently selected from 0, 1, 2, 3 or 4; and n₂, n₃ and n₄ are each independently selected from 0, 1 or 2; and
Substituent(s) in L₁, L₂, L₃, Ar₁ and Ar₂ are the same or different, and are each independently selected from deuterium, fluorine, cyano, aryl with 6 to 20 carbon atoms, heteroaryl with 3 to 20 carbon atoms, alkyl with 1 to 10 carbon atoms, alkoxy with 1 to 10 carbon atoms, haloalkyl with 1 to 10 carbon atoms, trialkylsilyl with 3 to 12 carbon atoms, triphenylsilyl, cycloalkyl with 3 to 10 carbon atoms, and deuteroalkyl with 1 to 10 carbon atoms; and optionally, any two adjacent substituents in Ar₁ and Ar₂ form a saturated or unsaturated 3- to 15-membered ring.

In the disclosure, the terms “optional” and “optionally” mean that the subsequently described event or circumstance may or may not occur. For example, “optionally, any two adjacent substituents in Ar₁ and Ar₂ form a saturated or unsaturated 3- to 15-membered ring”, which includes a scenario where any two adjacent substituents form a ring and a scenario where any two adjacent substituents are each independently present and do not form a ring. “Any two adjacent substituents” may include the condition that a same atom has two substituents, and may also include the condition that two adjacent atoms each have one substituent; when the same atom has two substituents, the two substituents may form a saturated or unsaturated spiro ring with the atom to which they are jointly connected; and when two adjacent atoms each have one substituent, the two substituents may be fused to form a ring.

In the disclosure, the adopted description modes “each . . . is independently”, “ . . . is respectively and independently” and “ . . . are each independently” can be interchanged, and should be understood in a broad sense, which means that in different groups, specific options expressed between the same symbols do not influence each other, or in a same group, specific options expressed between the same symbols do not influence each other. For example, the meaning of ″

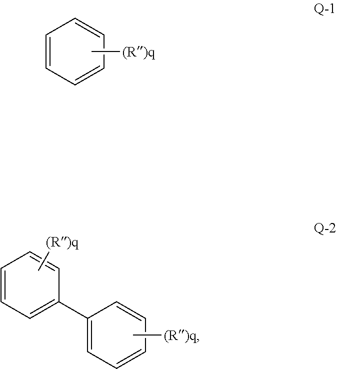

where each q is independently 0, 1, 2 or 3, and each R″ is independently selected from hydrogen, deuterium, fluorine and chlorine” is as follows: a formula Q-1 represents that q substituent(s) R″ exist on a benzene ring, each R″ can be the same or different, and options of each R″ do not influence each other; and a formula Q-2 represents that each benzene ring of biphenyl has q substituent(s) R″, the number q of the substituent(s) R″ on the two benzene rings can be the same or different, each R″ can be the same or different, and options of each R″ do not influence each other.

In the disclosure, the term such as “substituted or unsubstituted” means that a functional group described behind the term may have or not have a substituent (in the following, the substituent is collectively referred to as Rc in order to facilitate description). For example, “substituted or unsubstituted aryl” refers to aryl with a substituent Rc or aryl without a substituent. The above substituent, i.e. Rc, may be, for example, deuterium, fluorine, cyano, heteroaryl, aryl, trialkylsilyl, alkyl, haloalkyl, cycloalkyl, or the like. The number of substituent(s) may be one or more.

In the disclosure, “a plurality of” refers to two or more, e.g., two, three, four, five, six, etc.

In the disclosure, the number of carbon atoms in a substituted or unsubstituted functional group refers to the total number of carbon atoms of the group and all substituent(s) on the group. For example, if L₁ is substituted arylene with 12 carbon atoms, the number of all carbon atoms of the arylene and substituent(s) on the arylene is 12.

Hydrogen atoms in the structure of the compound of the disclosure include atoms of various isotopes of a hydrogen element, such as hydrogen (H), deuterium (D), or tritium (T). “D” in a structural formula of the compound of the disclosure represents deuteration.

In the disclosure, aryl refers to an optional functional group or substituent derived from an aromatic carbocyclic ring. The aryl may be monocyclic aryl (e.g., phenyl) or polycyclic aryl, in other words, the aryl can be monocyclic aryl, fused aryl, two or more monocyclic aryl conjugatedly connected through carbon-carbon bonds, monocyclic aryl and fused aryl which are conjugatedly connected through a carbon-carbon bond, or two or more fused aryl conjugatedly connected through carbon-carbon bonds. That is, unless otherwise noted, two or more aromatic groups conjugatedly connected through carbon-carbon bonds can also be regarded as the aryl of the disclosure. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. Examples of the aryl include, but are not limited to, phenyl, naphthyl, fluorenyl, spirobifluorenyl, anthryl, phenanthryl, biphenyl, terphenyl, triphenylene, perylenyl, benzo[9,10] phenanthryl, pyrenyl, benzofluoranthenyl, chrysenyl, and the like.

In the disclosure, the arylene involved refers to a divalent group formed by further loss of one or more hydrogen atoms from aryl.

In the disclosure, terphenyl includes

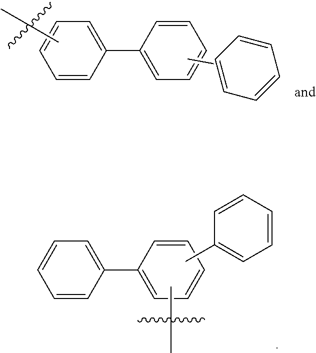

In the disclosure, the number of carbon atoms of the substituted or unsubstituted aryl (arylene) may be 6, 8, 10, 12, 13, 14, 15, 16, 17, 18, 19, 20, 21, 22, 23, 24, 25 or 30. In some embodiments, the substituted or unsubstituted aryl is substituted or unsubstituted aryl with 6 to 30 carbon atoms; in other embodiments, the substituted or unsubstituted aryl is substituted or unsubstituted aryl with 6 to 25 carbon atoms; in other embodiments, the substituted or unsubstituted aryl is substituted or unsubstituted aryl with 6 to 18 carbon atoms; and in other embodiments, the substituted or unsubstituted aryl is substituted or unsubstituted aryl with 6 to 15 carbon atoms.

In the disclosure, fluorenyl may be substituted with one or more substituents, and in the above case where the fluorenyl is substituted, the substituted fluorenyl may be

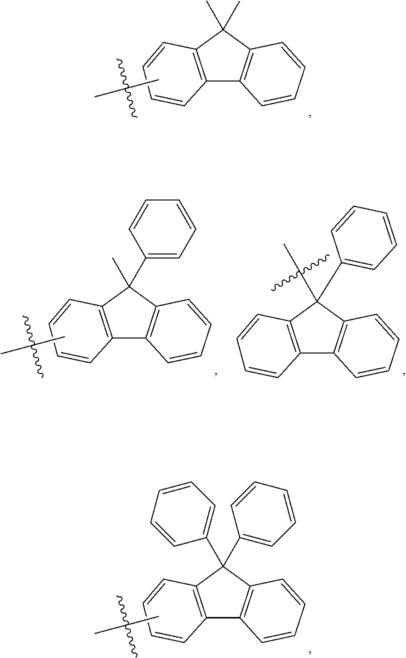

or the like, but is not limited to this.

In the disclosure, aryl as a substituent of L₁, L₂, L₃, Ar₁ and Ar₂ is, for example, but is not limited to, phenyl, naphthyl, phenanthryl, biphenyl, fluorenyl, dimethylfluorenyl or the like.

In the disclosure, heteroaryl refers to a monovalent aromatic ring containing 1, 2, 3, 4, 5 or 6 heteroatoms in the ring or its derivative, and the heteroatom may be one or more of B, O, N, P, Si, Se and S. The heteroaryl may be monocyclic heteroaryl or polycyclic heteroaryl, in other words, the heteroaryl may be a single aromatic ring system or a plurality of aromatic ring systems conjugatedly connected through carbon-carbon bonds, and any aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl and the like, but is not limited to this.

In the disclosure, the heteroarylene involved refers to a divalent or multivalent group formed by further loss of one or more hydrogen atoms from heteroaryl.

In the disclosure, the number of carbon atoms of the substituted or unsubstituted heteroaryl (heteroarylene) may be selected from 3, 4, 5, 6, 7, 8, 9, 10, 11, 12, 13, 14, 15, 16, 17, 18, 20, 21, 22, 23, 24, 25, 26, 27, 28, 29 and 30. In some embodiments, the substituted or unsubstituted heteroaryl is substituted or unsubstituted heteroaryl having a total number of carbon atoms being 12 to 18; and in other embodiments, the substituted or unsubstituted heteroaryl is substituted or unsubstituted heteroaryl having a total number of carbon atoms being 5 to 12.

In the disclosure, heteroaryl as a substituent of L₁, L₂, L₃, Ar₁ and Ar₂ is, for example, but not limited to, pyridyl, carbazolyl, dibenzothienyl, dibenzofuranyl, benzoxazolyl, benzothiazolyl, or benzimidazolyl.

In the disclosure, the substituted heteroaryl may be that one or two or more hydrogen atoms in the heteroaryl are substituted by groups such as a deuterium atom, a halogen group, cyano, aryl, heteroaryl, trialkylsilyl, alkyl, cycloalkyl, haloalkyl, and the like.

In the disclosure, the alkyl with 1 to 10 carbon atoms may include linear alkyl with 1 to 10 carbon atoms and branched alkyl with 3 to 10 carbon atoms. The number of carbon atoms of the alkyl may be, for example, 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, and the like.

In the disclosure, the halogen group may be, for example, fluorine, chlorine, bromine or iodine.

In the disclosure, specific examples of trialkylsilyl include, but are not limited to, trimethylsilyl, triethylsilyl, and the like.

In the disclosure, specific examples of haloalkyl include, but are not limited to, trifluoromethyl.

In the disclosure, the number of carbon atoms of cycloalkyl with 3 to 10 carbon atoms may be, for example, 3, 4, 5, 6, 7, 8, or 10. Specific examples of the cycloalkyl include, but are not limited to, cyclopentyl, cyclohexyl, and adamantyl.

In the disclosure, the number of carbon atoms of deuteroalkyl with 1 to 10 carbon atoms is, for example, 1, 2, 3, 4, 5, 6, 7, 8 or 10. Specific examples of the deuteroalkyl include, but are not limited to, trideuteromethyl.

In the disclosure, the number of carbon atoms of haloalkyl with 1 to 10 carbon atoms is, for example, 1, 2, 3, 4, 5, 6, 7, 8 or 10. Specific examples of the haloalkyl include, but are not limited to, trifluoromethyl.

In the disclosure, a ring system formed by n atoms is an n-membered ring. For example, phenyl is a 6-membered ring. A 3- to 15-membered ring refers to a cyclic group with 3 to 15 ring atoms. The 3- to 15-membered ring is, for example, cyclopentane, cyclohexane, a fluorene ring, a benzene ring or the like.

In the disclosure, [symbol] refers to a chemical bond interconnecting with other groups.

In the disclosure, an unpositioned connecting bond refers to a single bond “ [symbol] ” extending from a ring system, which means that one end of the connecting bond can be connected with any position in the ring system through which the bond penetrates, and the other end of the connecting bond is connected with the remaining part of a compound molecule. For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1) to (f-10)

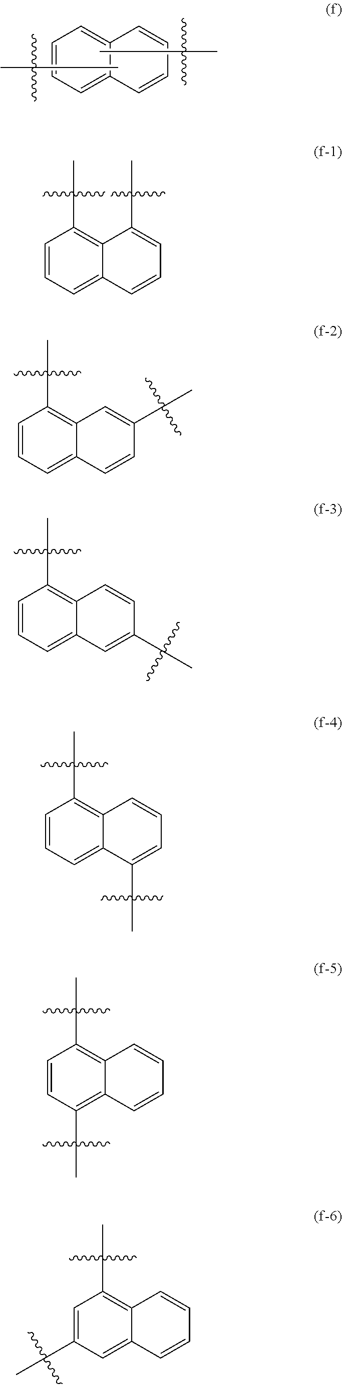

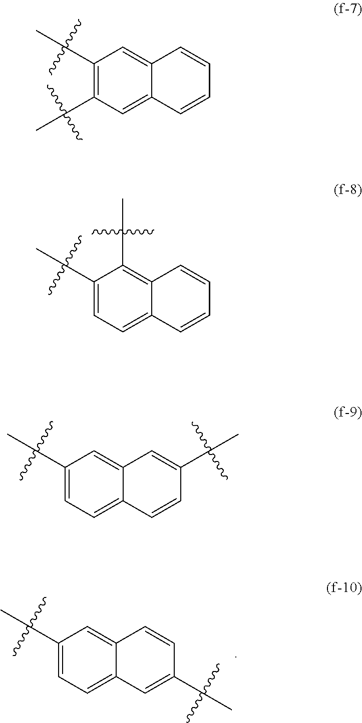 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by formulae (X′-1) to (X′-4):

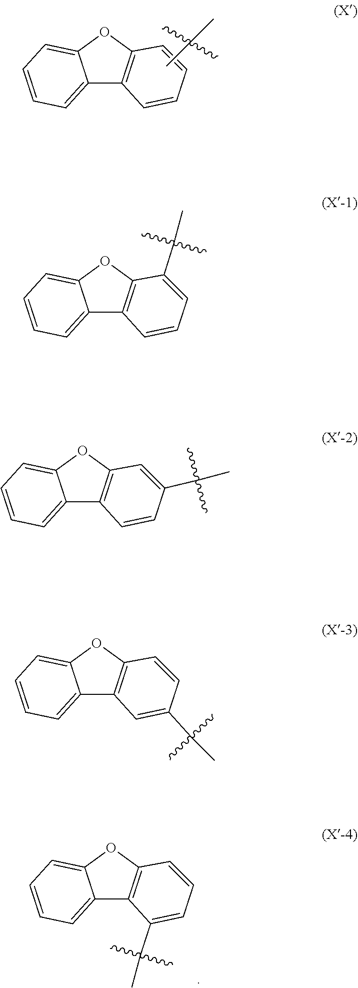

An unpositioned substituent in the disclosure refers to a substituent connected through a single bond extending from the center of a ring system, which means that the substituent can be connected to any possible position in the ring system. For example, as shown in the following formula (Y), a substituent R′ represented by the formula (Y) is connected with a quinoline ring through one unpositioned connecting bond, and its meaning includes any one possible connecting mode represented by formula (Y-1) to formula (Y-7):

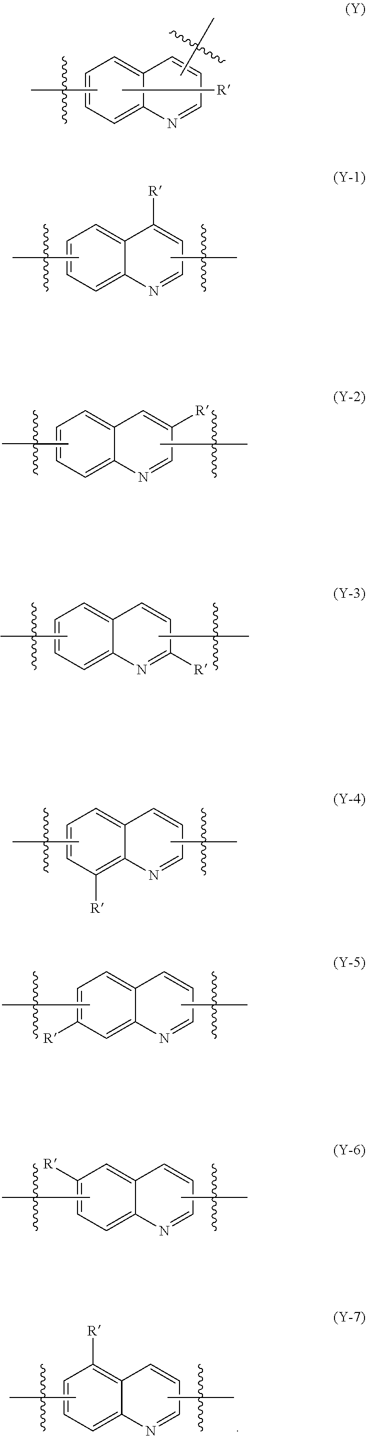

In some embodiments, the formula 1 is specifically selected from structures represented by formula (1-1) to formula (1-7):

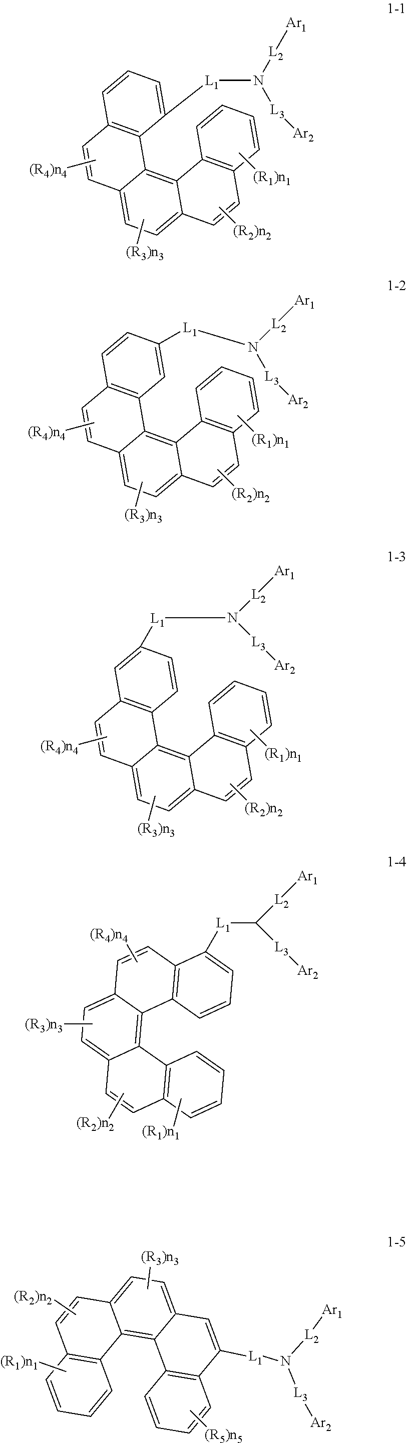

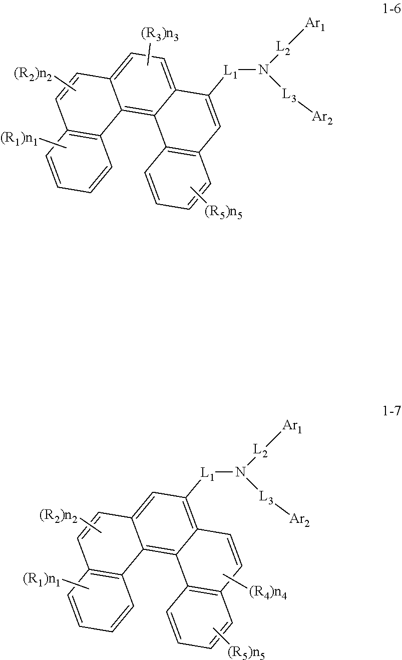

In some embodiments, Ar₁ and Ar₂ are each independently selected from substituted or unsubstituted aryl with 6, 7, 8, 9, 10, 11, 12, 13, 14, 15, 16, 17, 18, 19, 20, 21, 22, 23, 24, 25, 26, 27, 28, 29 or 30 carbon atoms, and substituted or unsubstituted heteroaryl with 3, 4, 5, 6, 7, 8, 9, 10, 12, 13, 14, 15, 16, 17, 18, 19, 20, 21, 22, 23, 24, 25, 26, 27, 28, 29 or 30 carbon atoms.

In some embodiments, Ar₁ and Ar₂ are each independently selected from substituted or unsubstituted aryl with 6 to 25 carbon atoms, and substituted or unsubstituted heteroaryl with 12 to 18 carbon atoms.

Optionally, Ar₁ and Ar₂ are each independently selected from substituted or unsubstituted phenyl, substituted or unsubstituted naphthyl, substituted or unsubstituted biphenyl, substituted or unsubstituted fluorenyl, substituted or unsubstituted terphenyl, substituted or unsubstituted phenanthryl, substituted or unsubstituted anthryl, substituted or unsubstituted pyrenyl, substituted or unsubstituted triphenylene, substituted or unsubstituted dibenzofuranyl, substituted or unsubstituted dibenzothienyl, and substituted or unsubstituted carbazolyl.

In some embodiments, substituent(s) in Ar₁ and Ar₂ are each independently selected from deuterium, fluorine, cyano, haloalkyl with 1 to 4 carbon atoms, deuteroalkyl with 1 to 4 carbon atoms, alkyl with 1 to 4 carbon atoms, cycloalkyl with 5 to 10 carbon atoms, aryl with 6 to 12 carbon atoms, heteroaryl with 5 to 12 carbon atoms, and trialkylsilyl with 3 to 8 carbon atoms, and optionally, any two adjacent substituents in Ar₁ and Ar₂ form a benzene ring or a fluorene ring.

Optionally, the substituent(s) in Ar₁ and Ar₂ are each independently selected from deuterium, fluorine, cyano, methyl, ethyl, isopropyl, tert-butyl, trideuteromethyl, cyclopentyl, cyclohexyl, phenyl, naphthyl, biphenyl, pyridyl, dibenzofuranyl, dibenzothienyl or trimethylsilyl; and optionally, any two adjacent substituents in Ar₁ and Ar₂ form a benzene ring, cyclopentane, cyclohexane or fluorene ring.

In some embodiments, Ar₁ and Ar₂ are each independently selected from a substituted or unsubstituted group W, the unsubstituted group W being selected from the group consisting of:

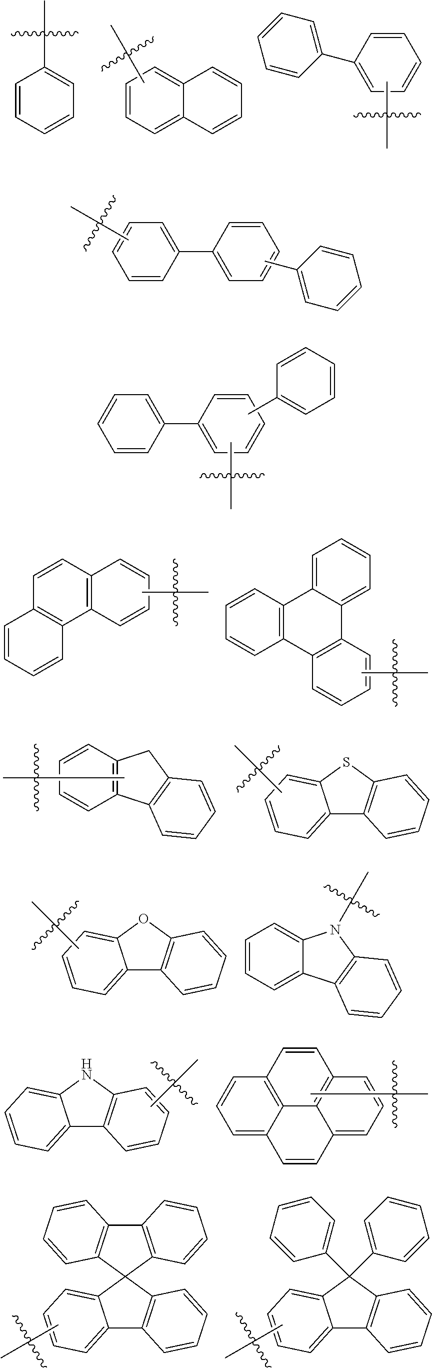

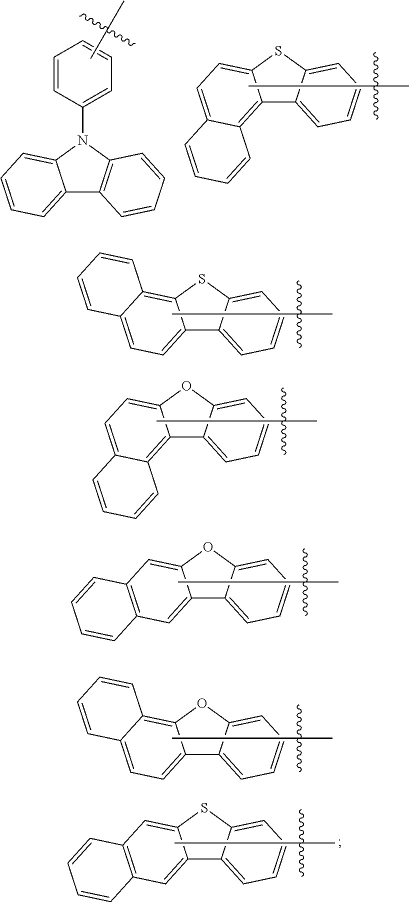

the substituted group W has one or two or more substituents, and the substituent(s) are each independently selected from deuterium, fluorine, cyano, trimethylsilyl, trifluoromethyl, cyclopentyl, cyclohexyl, methyl, ethyl, isopropyl, tert-butyl, phenyl, naphthyl, pyridyl, dibenzofuranyl, dibenzothienyl, and carbazolyl, and when the number of substituents in the group W is greater than 1, the substituents are the same or different.

In some embodiments, Ar₁ and Ar₂ are each independently selected from:

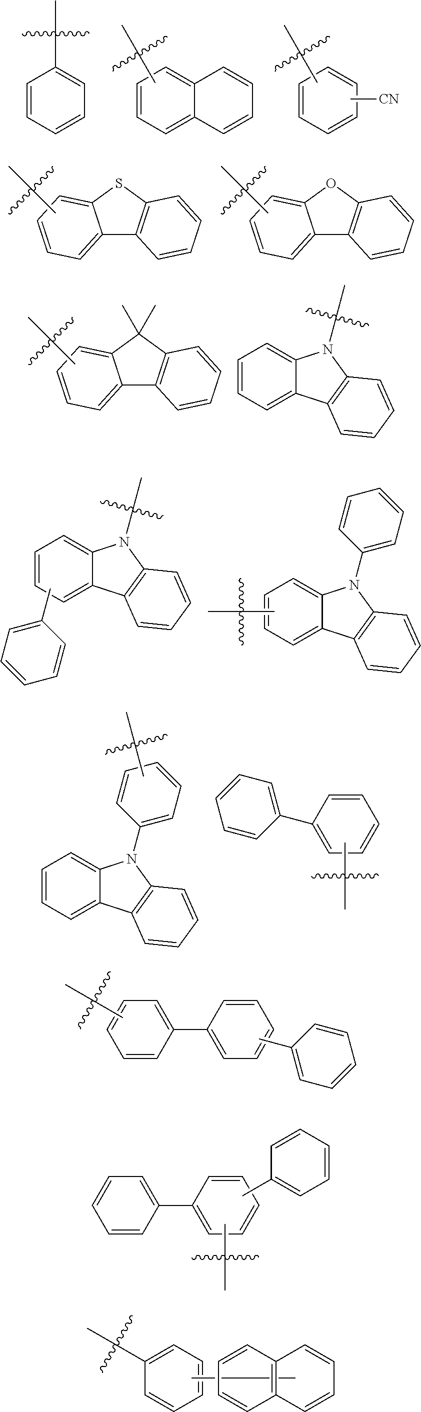

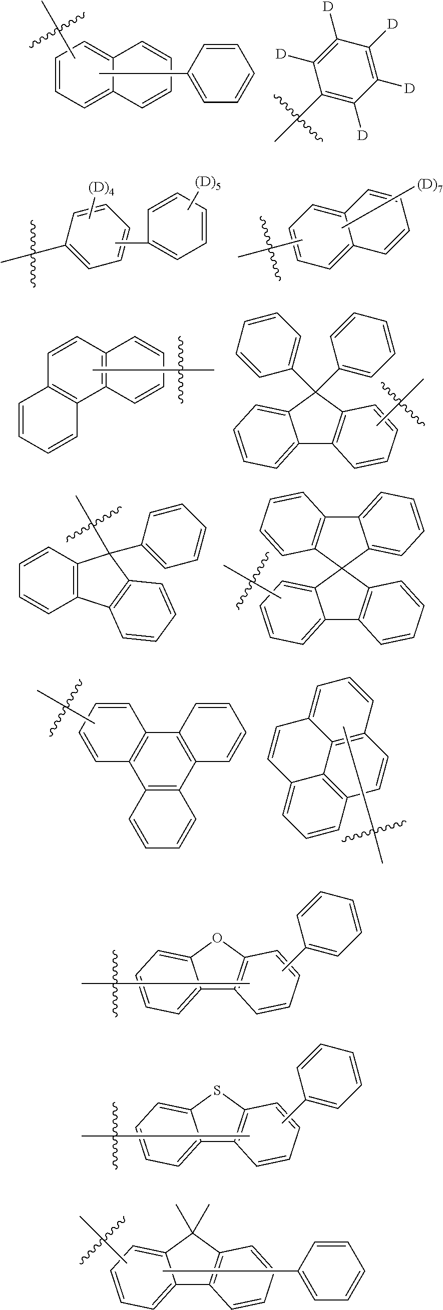

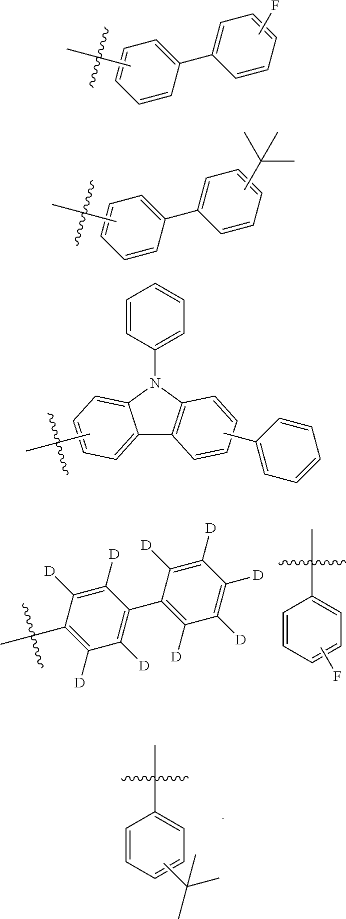

In some embodiments, Ar₁ and Ar₂ are each independently selected from:

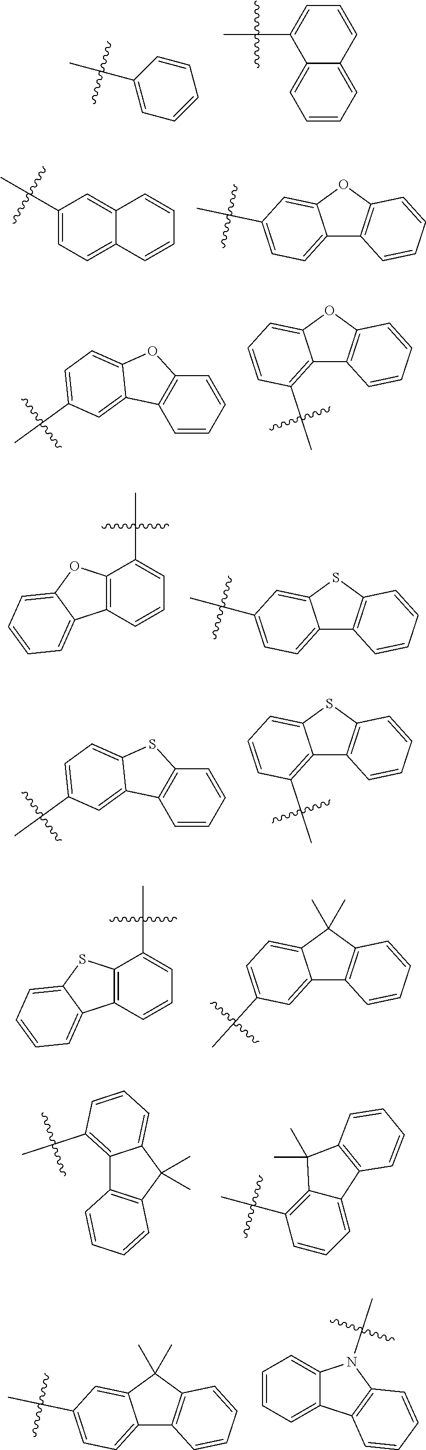

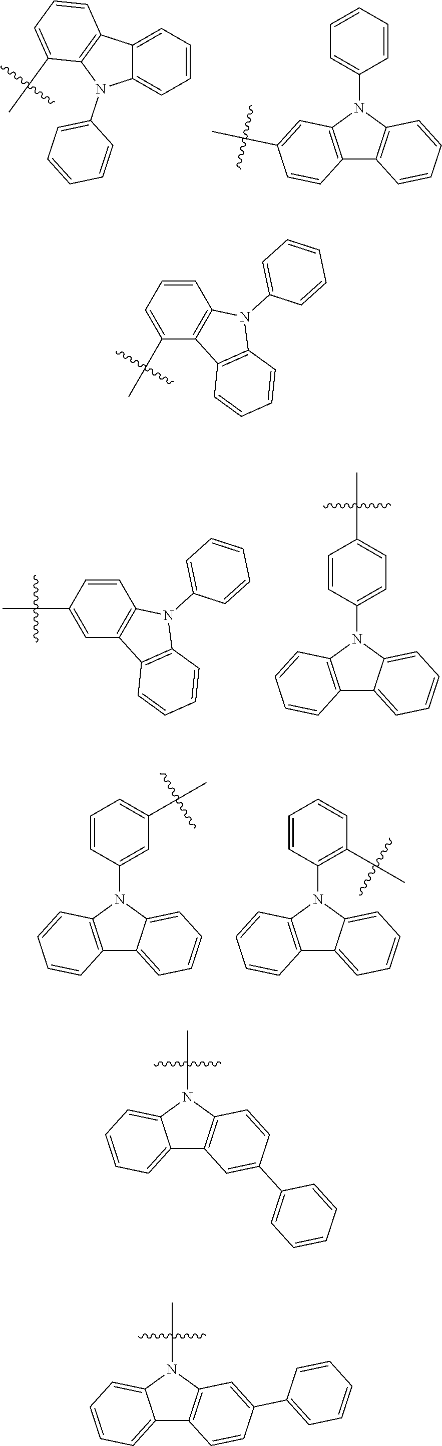

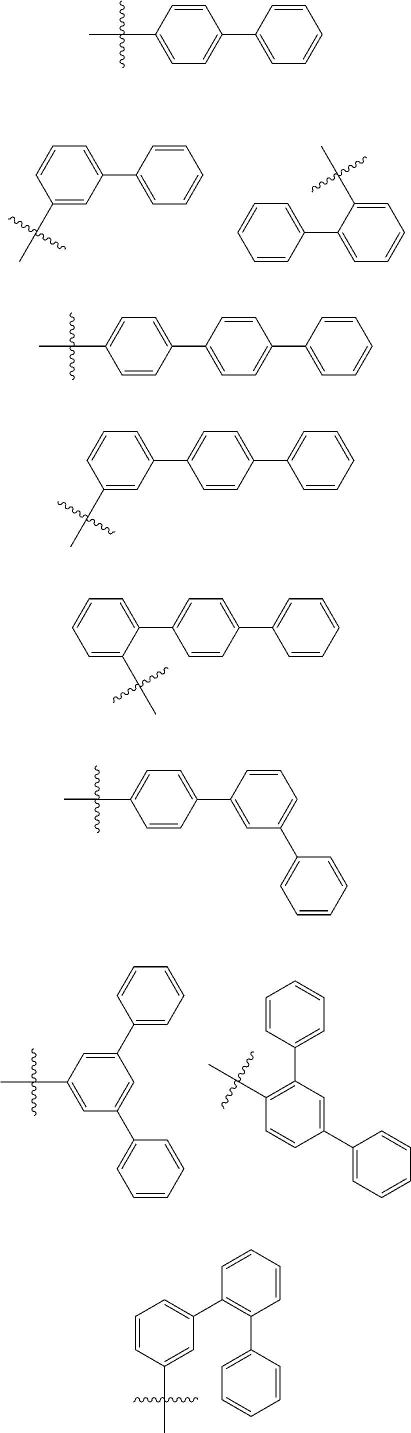

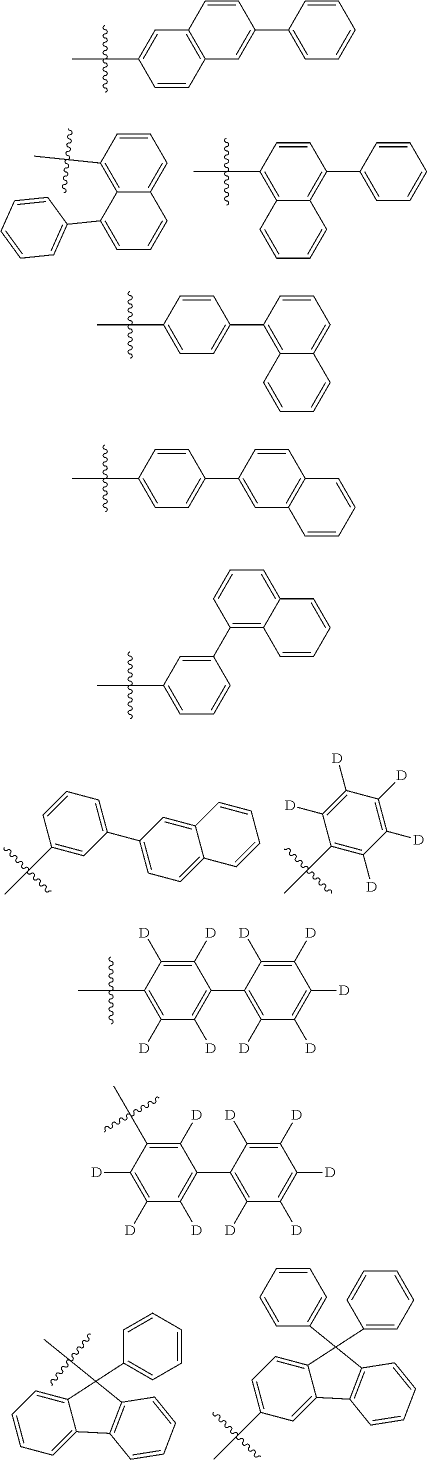

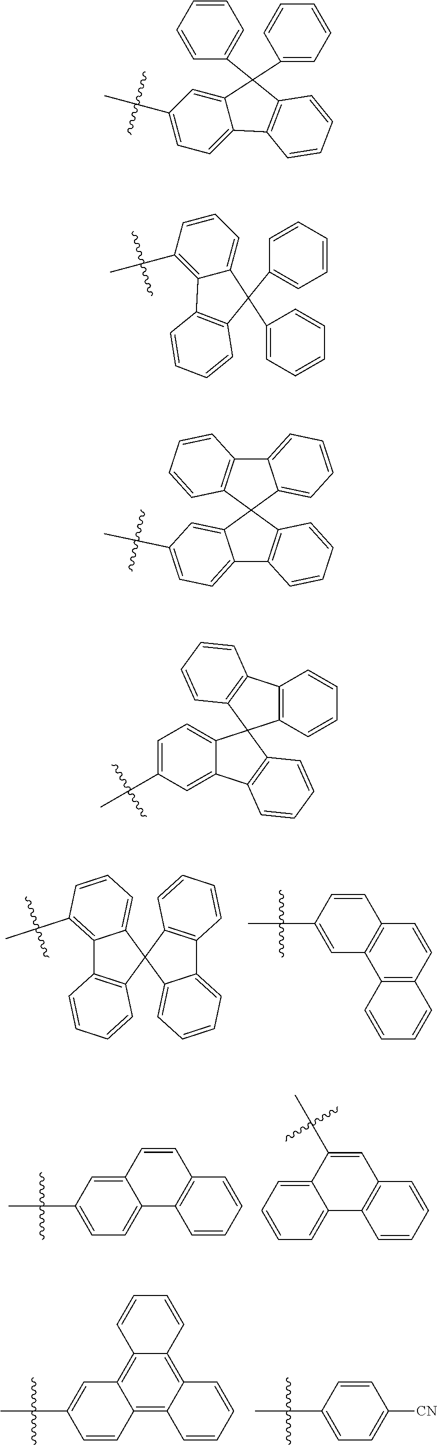

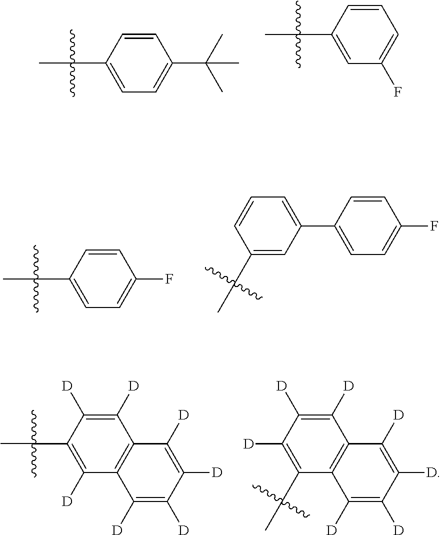

In some embodiments, L₁, L₂ and L₃ are each independently selected from a single bond, substituted or unsubstituted arylene with 6, 7, 8, 9, 10, 11, 12, 13, 14, 15, 16, 17, 18, 19, 20, 21, 22, 23, 24, 25, 26, 27, 28, 29 or 30 carbon atoms, and substituted or unsubstituted heteroarylene with 3, 4, 5, 6, 7, 8, 9, 10, 12, 13, 14, 15, 16, 17, 18, 19, 20, 21, 22, 23, 24, 25, 26, 27, 28, 29 or 30 carbon atoms.

In some embodiments, L₁, L₂ and L₃ are each independently selected from a single bond, substituted or unsubstituted arylene with 6 to 15 carbon atoms, and substituted or unsubstituted heteroarylene with 12 to 18 carbon atoms.

In some embodiments, substituent(s) in L₁, L₂ and L₃ are each independently selected from deuterium, fluorine, cyano, haloalkyl with 1 to 4 carbon atoms, deuteroalkyl with 1 to 4 carbon atoms, alkyl with 1 to 4 carbon atoms, aryl with 6 to 10 carbon atoms, and trialkylsilyl with 3 to 8 carbon atoms.

In some embodiments, L₁, L₂ and L₃ are each independently selected from a single bond, substituted or unsubstituted phenylene, substituted or unsubstituted naphthylene, substituted or unsubstituted biphenylene, substituted or unsubstituted fluorenylene, substituted or unsubstituted phenanthrylene, substituted or unsubstituted anthrylene, substituted or unsubstituted carbazolylene, substituted or unsubstituted dibenzothienylene, and substituted or unsubstituted dibenzofurylene.

Optionally, the substituent(s) in L₁, L₂ and L₃ are each independently selected from deuterium, fluorine, cyano, trimethylsilyl, trideuteromethyl, trifluoromethyl, methyl, ethyl, isopropyl, tert-butyl, phenyl and naphthyl.

In some embodiments, L₁ is selected from a single bond or the group consisting of:

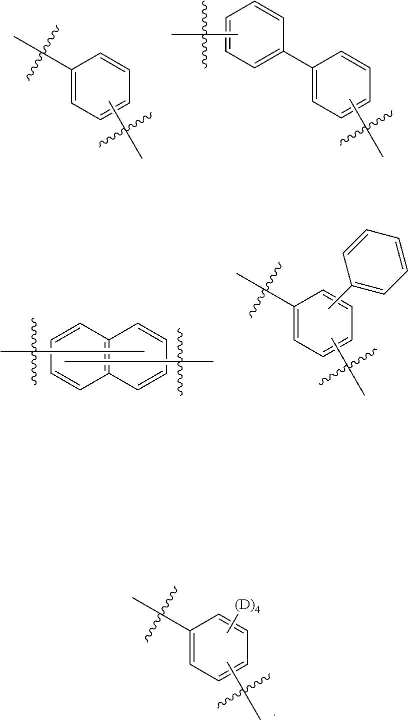

In some embodiments, L₂ and L₃ are each independently selected from a single bond or the group consisting of:

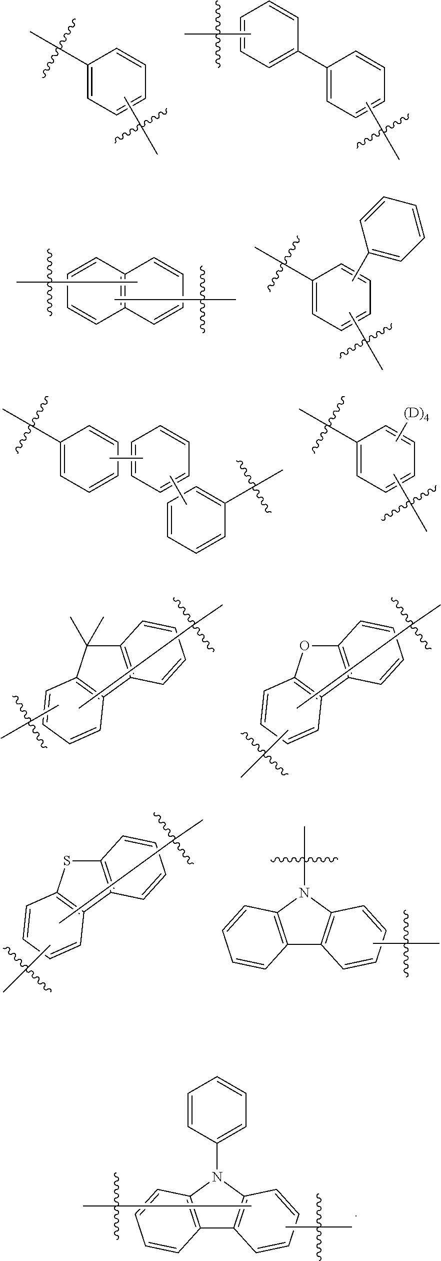

In some embodiments, L₁ is selected from a single bond, phenylene, deuterophenylene, and naphthylene.

In some embodiments, L₁ is selected from a single bond or the following groups:

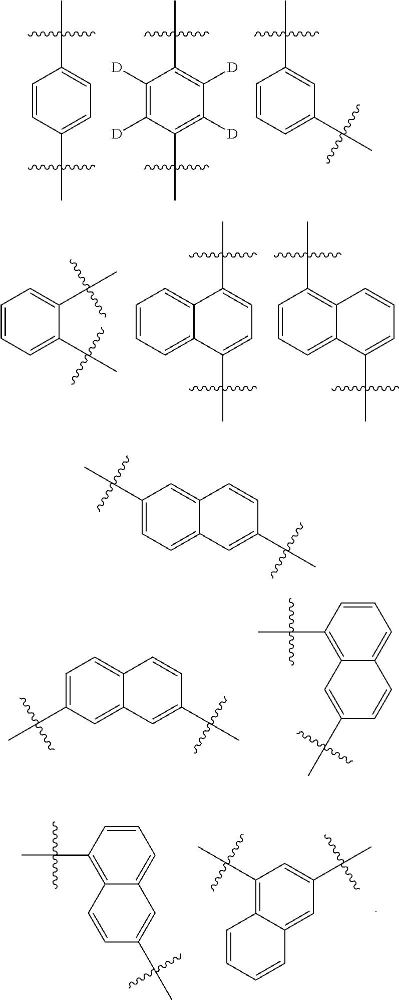

In some embodiments, L₂ and L₃ are each independently selected from a single bond, substituted or unsubstituted phenylene, substituted or unsubstituted naphthylene, substituted or unsubstituted biphenylene, substituted or unsubstituted carbazolylene, substituted or unsubstituted dibenzothienylene, and substituted or unsubstituted dibenzofurylene.

Optionally, substituent(s) in L₂ and L₃ are each independently selected from deuterium, fluorine, cyano, trimethylsilyl, trideuteromethyl, trifluoromethyl, methyl, ethyl, isopropyl, tert-butyl and phenyl.

In some embodiments, L₂ and L₃ are each independently selected from a single bond or the following groups:

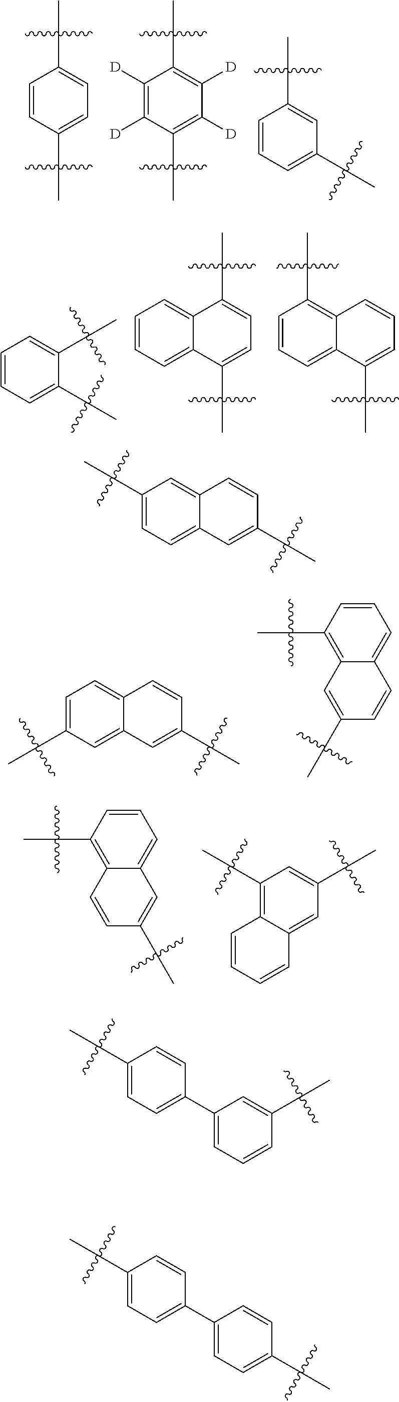

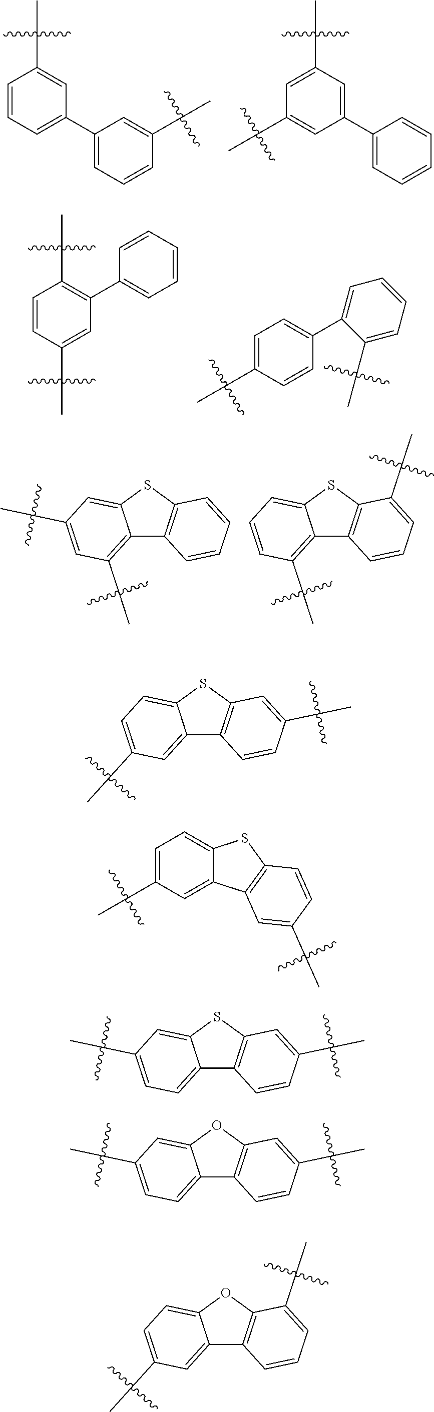

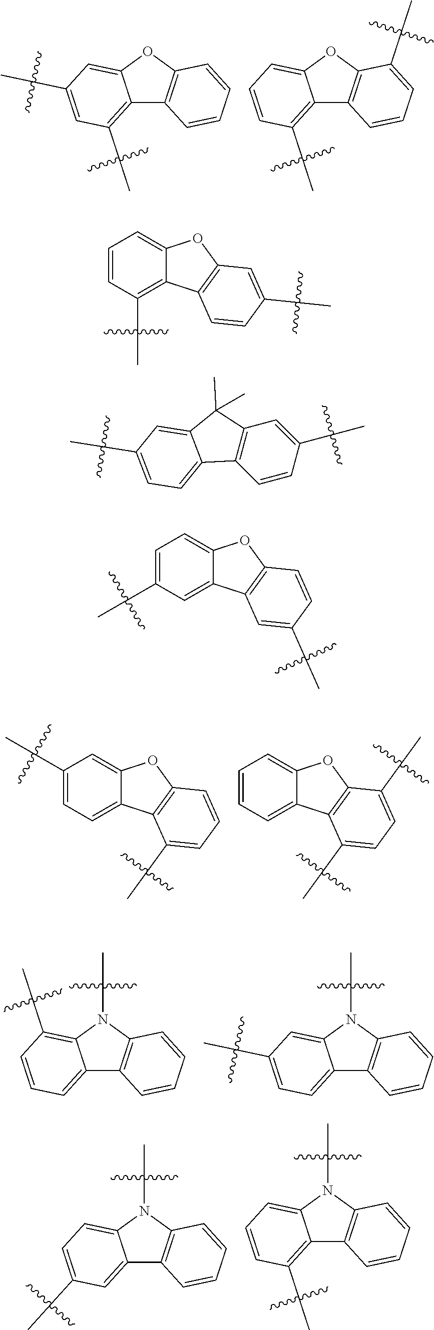

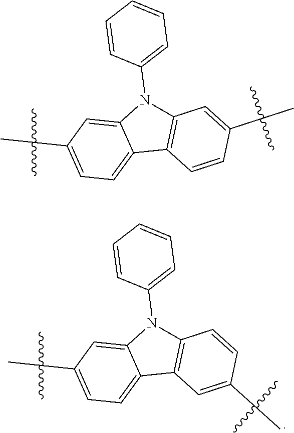 [SMILES: *c1ccc2c3ccc(*)cc3n(-c3ccccc3)c2c1 ; *c1ccc2c(c1)c1ccc(*)cc1n2-c1ccccc1]

In some embodiments,

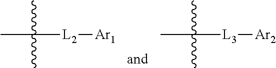

are each independently selected from the following groups:

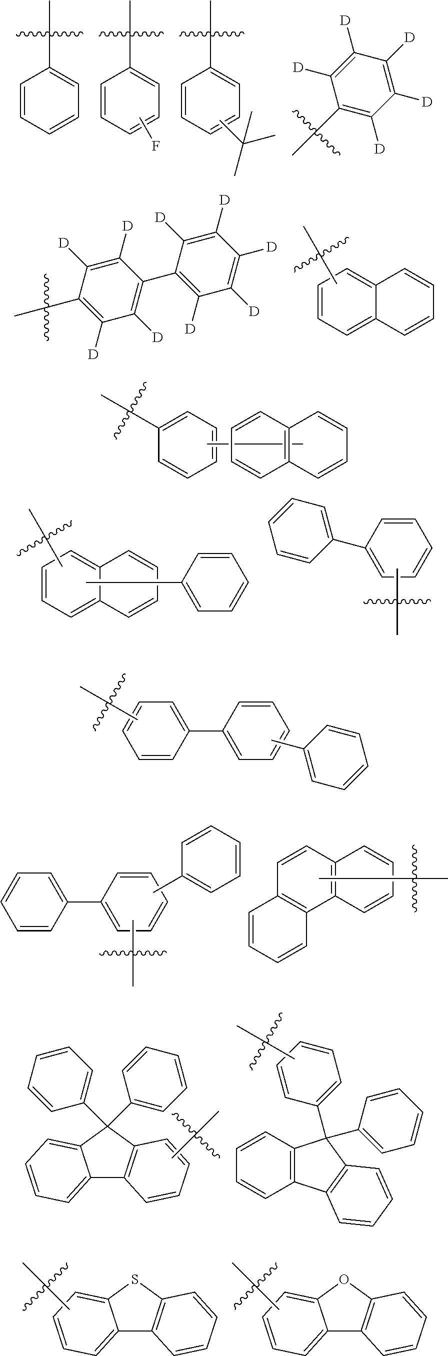

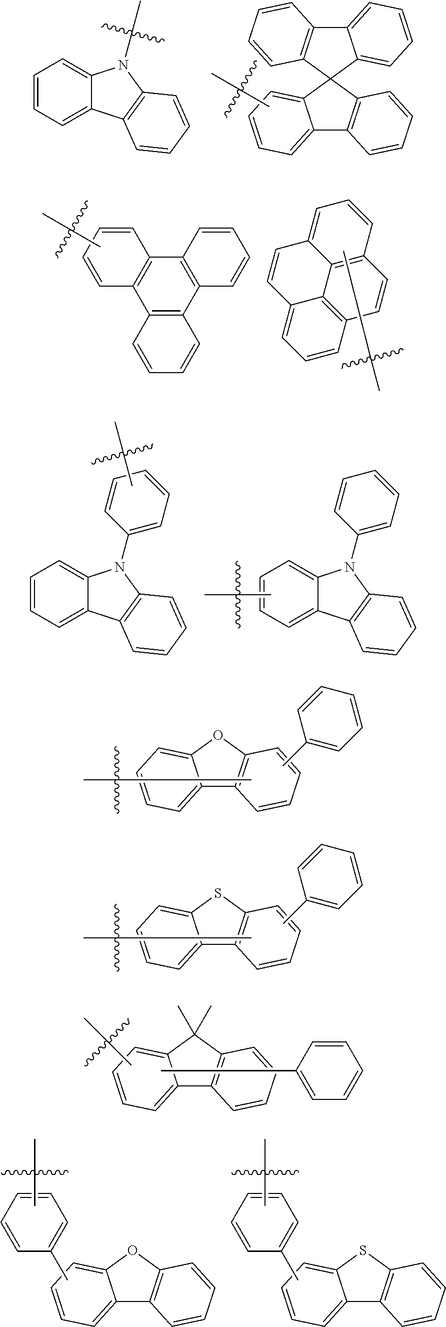

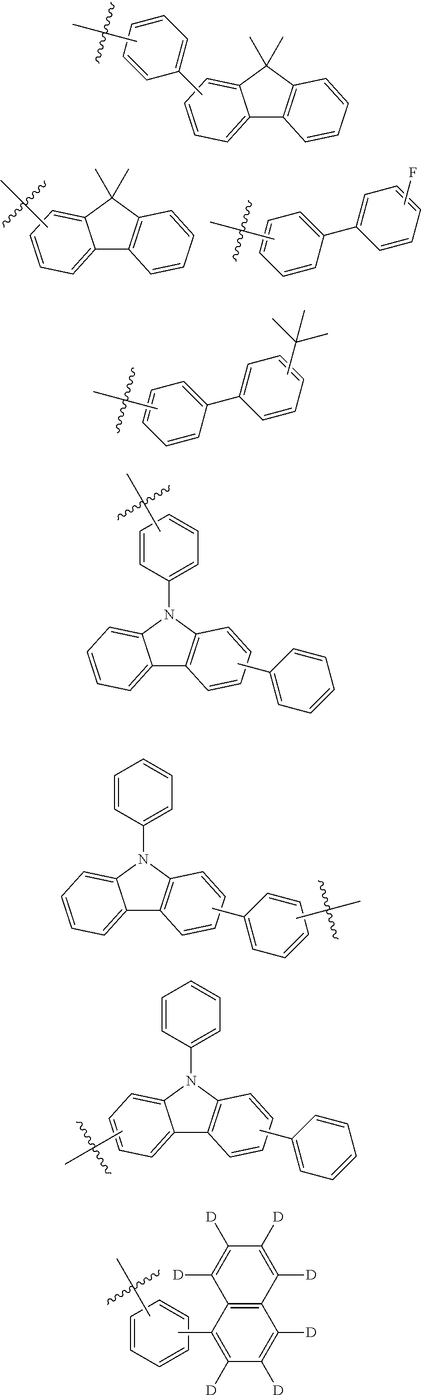

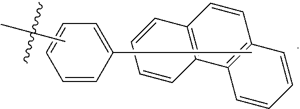

In some embodiments, R₁, R₂, R₃, R₄, and R₅ are the same or different, and are each independently selected from deuterium, cyano, trideuteromethyl, trimethylsilyl, trifluoromethyl, methyl, ethyl, isopropyl, tert-butyl, phenyl, and naphthyl.

In some embodiments,

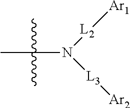

is selected from the following groups:

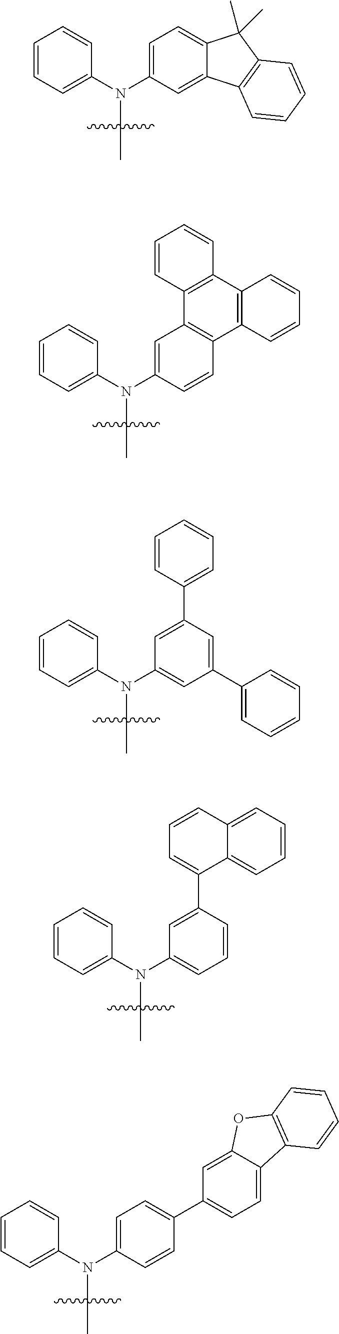 [SMILES: *N(c1ccccc1)c1ccc2c(c1)-c1ccccc1C2(C)C ; *N(c1ccccc1)c1ccc2c3ccccc3c3ccccc3c2c1 ; *N(c1ccccc1)c1cc(-c2ccccc2)cc(-c2ccccc2)c1 ; *N(c1ccccc1)c1cccc(-c2cccc3ccccc23)c1 ; *N(c1ccccc1)c1ccc(-c2ccc3c(c2)oc2ccccc23)cc1]

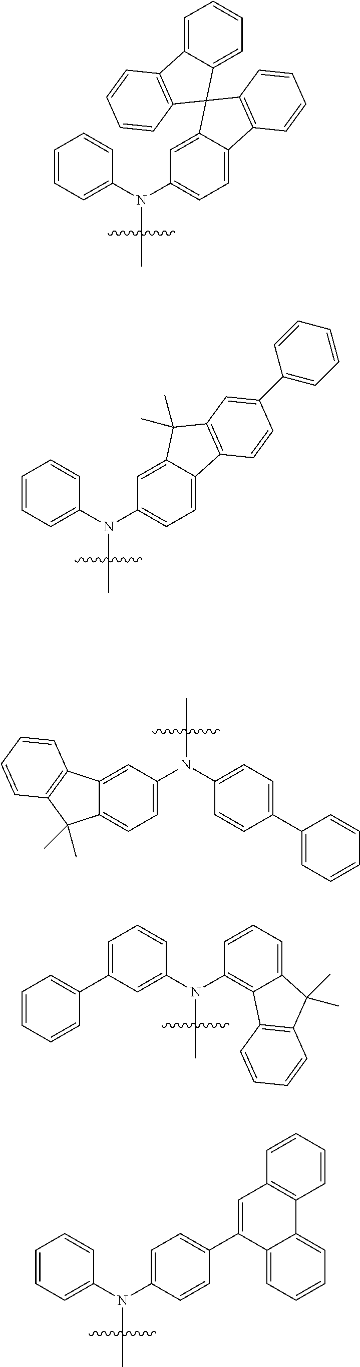 [SMILES: *N(c1ccccc1)c1ccc2c(c1)C1(c3ccccc3-c3ccccc31)c1ccccc1-2 ; *N(c1ccccc1)c1ccc2c(c1)C(C)(C)c1cc(-c3ccccc3)ccc1-2 ; *N(c1ccc(-c2ccccc2)cc1)c1ccc2c(c1)-c1ccccc1C2(C)C ; *N(c1cccc(-c2ccccc2)c1)c1cccc2c1-c1ccccc1C2(C)C ; *N(c1ccccc1)c1ccc(-c2cc3ccccc3c3ccccc23)cc1]

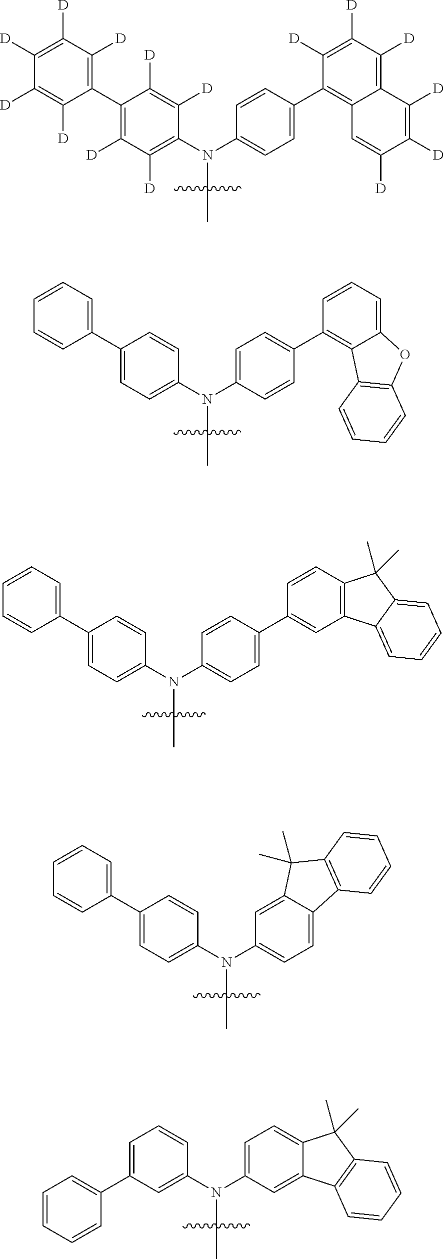 [SMILES: *N(c1ccc(-c2c([2H])c([2H])c([2H])c3c([2H])c([2H])c([2H])cc23)cc1)c1c([2H])c([2H])c(-c2c([2H])c([2H])c([2H])c([2H])c2[2H])c([2H])c1[2H] ; *N(c1ccc(-c2ccccc2)cc1)c1ccc(-c2cccc3oc4ccccc4c23)cc1 ; *N(c1ccc(-c2ccccc2)cc1)c1ccc(-c2ccc3c(c2)-c2ccccc2C3(C)C)cc1 ; *N(c1ccc(-c2ccccc2)cc1)c1ccc2c(c1)C(C)(C)c1ccccc1-2 ; *N(c1cccc(-c2ccccc2)c1)c1ccc2c(c1)-c1ccccc1C2(C)C]

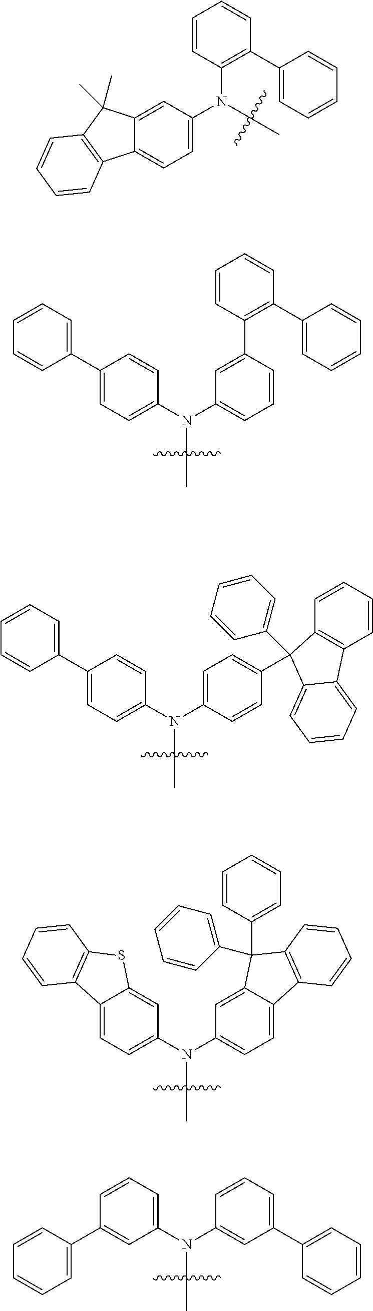 [SMILES: *N(c1ccc2c(c1)C(C)(C)c1ccccc1-2)c1ccccc1-c1ccccc1 ; *N(c1ccc(-c2ccccc2)cc1)c1cccc(-c2ccccc2-c2ccccc2)c1 ; *N(c1ccc(-c2ccccc2)cc1)c1ccc(C2(c3ccccc3)c3ccccc3-c3ccccc32)cc1 ; *N(c1ccc2c(c1)C(c1ccccc1)(c1ccccc1)c1ccccc1-2)c1ccc2c(c1)sc1ccccc12 ; *N(c1cccc(-c2ccccc2)c1)c1cccc(-c2ccccc2)c1]

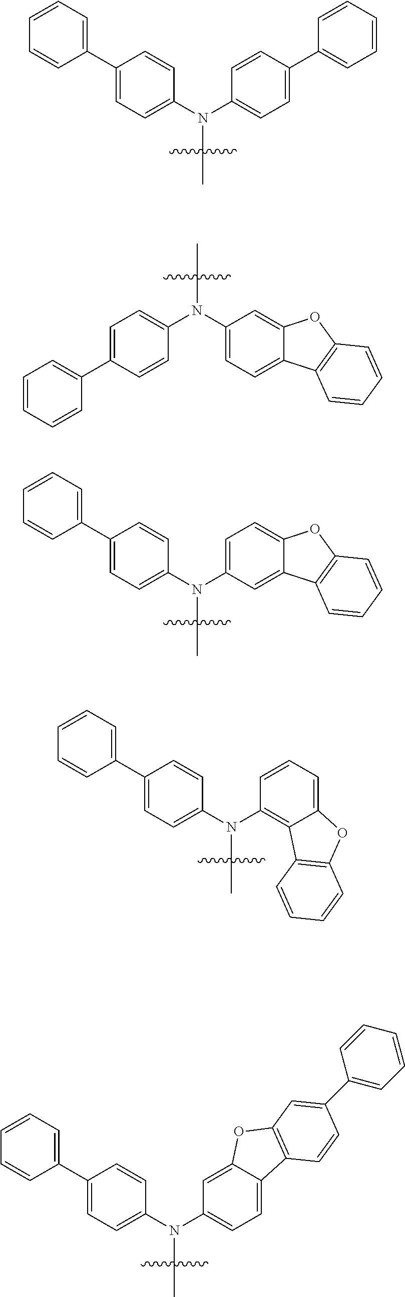

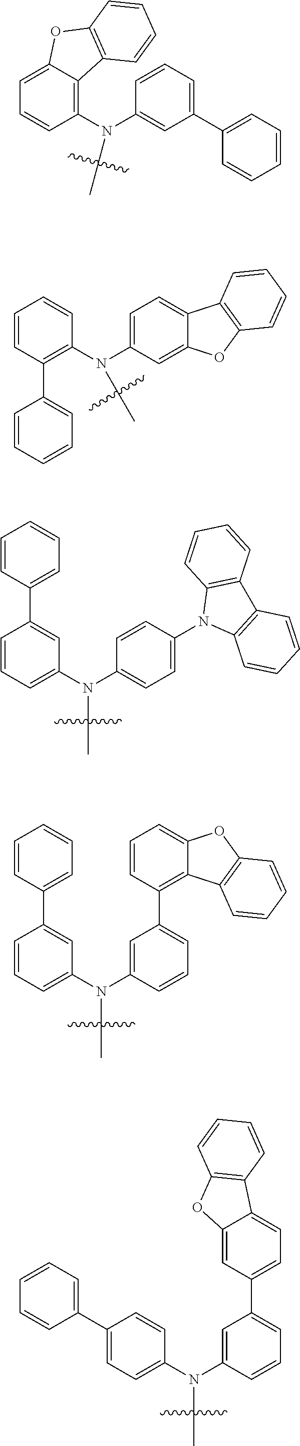 [SMILES: *N(c1cccc(-c2ccccc2)c1)c1cccc2oc3ccccc3c12 ; *N(c1ccc2c(c1)oc1ccccc12)c1ccccc1-c1ccccc1 ; *N(c1ccc(-n2c3ccccc3c3ccccc32)cc1)c1cccc(-c2ccccc2)c1 ; *N(c1cccc(-c2ccccc2)c1)c1cccc(-c2cccc3oc4ccccc4c23)c1 ; *N(c1ccc(-c2ccccc2)cc1)c1cccc(-c2ccc3c(c2)oc2ccccc23)c1]

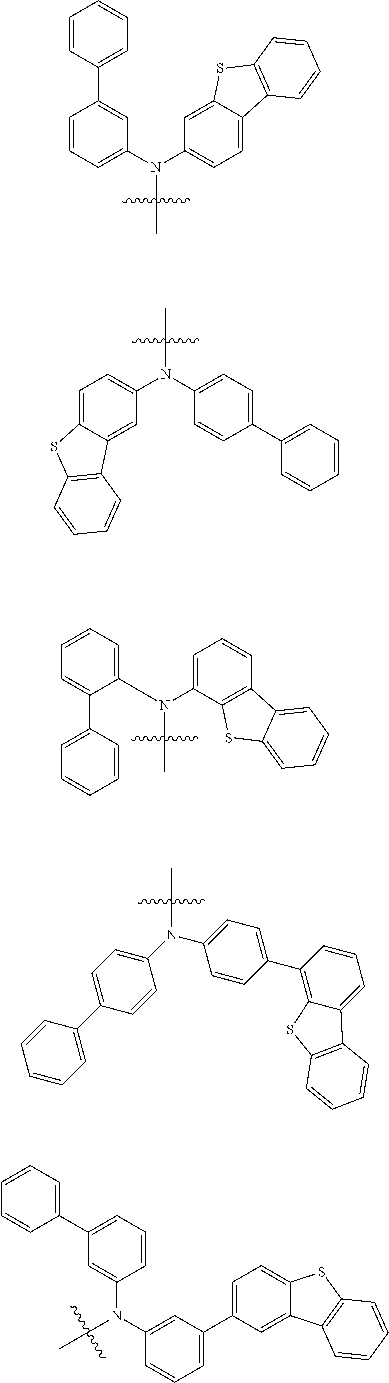 [SMILES: *N(c1cccc(-c2ccccc2)c1)c1ccc2c(c1)sc1ccccc12 ; *N(c1ccc(-c2ccccc2)cc1)c1ccc2sc3ccccc3c2c1 ; *N(c1ccccc1-c1ccccc1)c1cccc2c1sc1ccccc12 ; *N(c1ccc(-c2ccccc2)cc1)c1ccc(-c2cccc3c2sc2ccccc23)cc1 ; *N(c1cccc(-c2ccccc2)c1)c1cccc(-c2ccc3sc4ccccc4c3c2)c1]

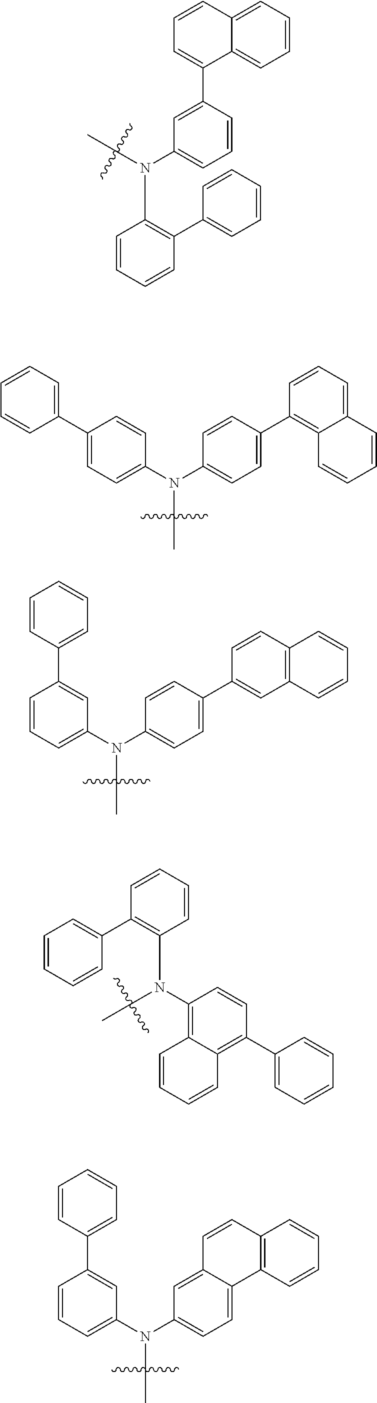 [SMILES: *N(c1cccc(-c2cccc3ccccc23)c1)c1ccccc1-c1ccccc1 ; *N(c1ccc(-c2ccccc2)cc1)c1ccc(-c2cccc3ccccc23)cc1 ; *N(c1ccc(-c2ccc3ccccc3c2)cc1)c1cccc(-c2ccccc2)c1 ; *N(c1ccccc1-c1ccccc1)c1ccc(-c2ccccc2)c2ccccc12 ; *N(c1cccc(-c2ccccc2)c1)c1ccc2c(ccc3ccccc32)c1]

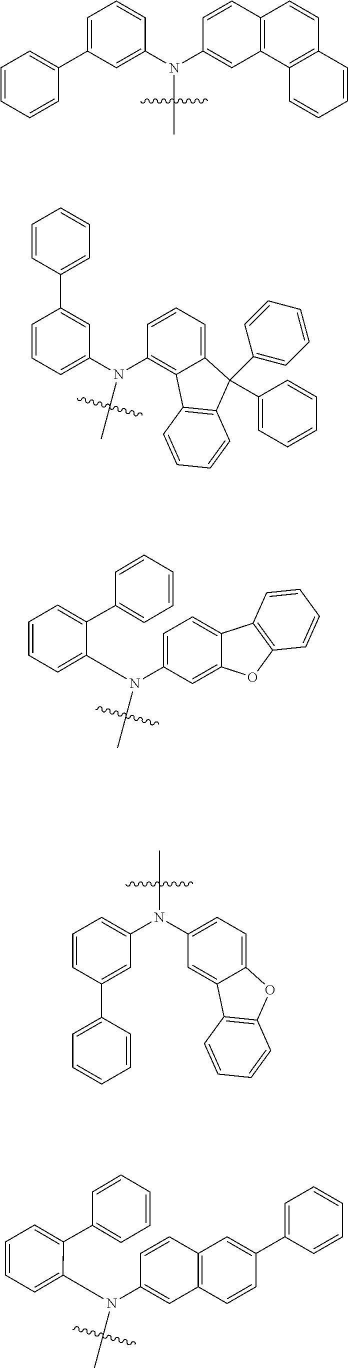 [SMILES: *N(c1cccc(-c2ccccc2)c1)c1ccc2ccc3ccccc3c2c1 ; *N(c1cccc(-c2ccccc2)c1)c1cccc2c1-c1ccccc1C2(c1ccccc1)c1ccccc1 ; *N(c1ccc2c(c1)oc1ccccc12)c1ccccc1-c1ccccc1 ; *N(c1cccc(-c2ccccc2)c1)c1ccc2oc3ccccc3c2c1 ; *N(c1ccc2cc(-c3ccccc3)ccc2c1)c1ccccc1-c1ccccc1]

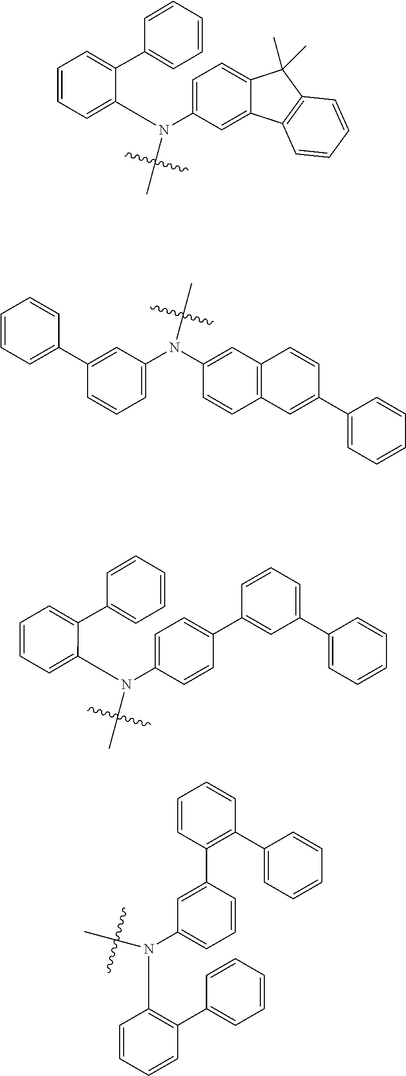 [SMILES: *N(c1ccc2c(c1)-c1ccccc1C2(C)C)c1ccccc1-c1ccccc1 ; *N(c1cccc(-c2ccccc2)c1)c1ccc2cc(-c3ccccc3)ccc2c1 ; *N(c1ccc(-c2cccc(-c3ccccc3)c2)cc1)c1ccccc1-c1ccccc1 ; *N(c1cccc(-c2ccccc2-c2ccccc2)c1)c1ccccc1-c1ccccc1]

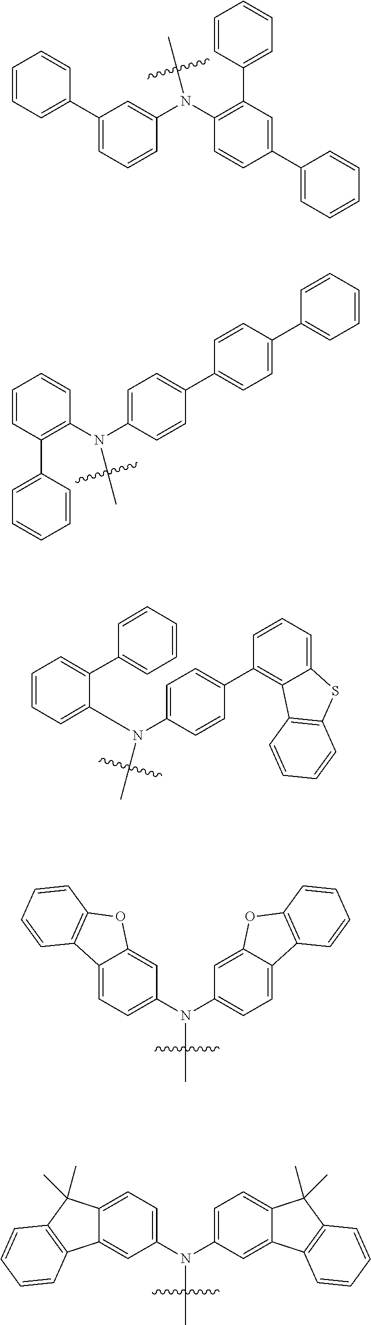 [SMILES: *N(c1cccc(-c2ccccc2)c1)c1ccc(-c2ccccc2)cc1-c1ccccc1 ; *N(c1ccc(-c2ccc(-c3ccccc3)cc2)cc1)c1ccccc1-c1ccccc1 ; *N(c1ccc(-c2cccc3sc4ccccc4c23)cc1)c1ccccc1-c1ccccc1 ; *N(c1ccc2c(c1)oc1ccccc12)c1ccc2c(c1)oc1ccccc12 ; *N(c1ccc2c(c1)-c1ccccc1C2(C)C)c1ccc2c(c1)-c1ccccc1C2(C)C]

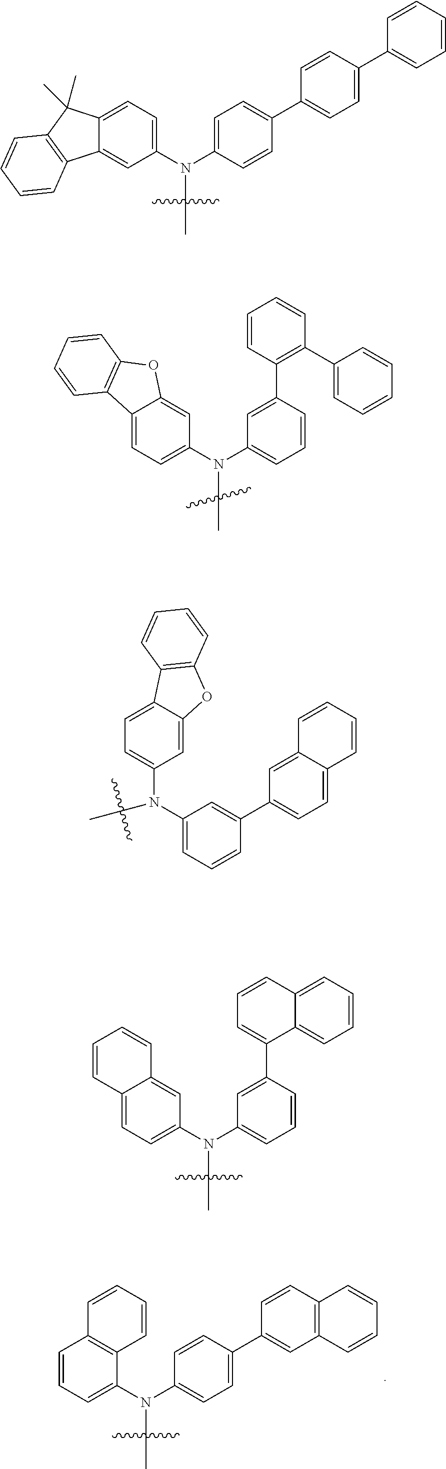 [SMILES: *N(c1ccc(-c2ccc(-c3ccccc3)cc2)cc1)c1ccc2c(c1)-c1ccccc1C2(C)C ; *N(c1cccc(-c2ccccc2-c2ccccc2)c1)c1ccc2c(c1)oc1ccccc12 ; *N(c1cccc(-c2ccc3ccccc3c2)c1)c1ccc2c(c1)oc1ccccc12 ; *N(c1cccc(-c2cccc3ccccc23)c1)c1ccc2ccccc2c1 ; *N(c1ccc(-c2ccc3ccccc3c2)cc1)c1cccc2ccccc12]

In some embodiments, the arylamine compound is selected from the group consisting of the following compounds:

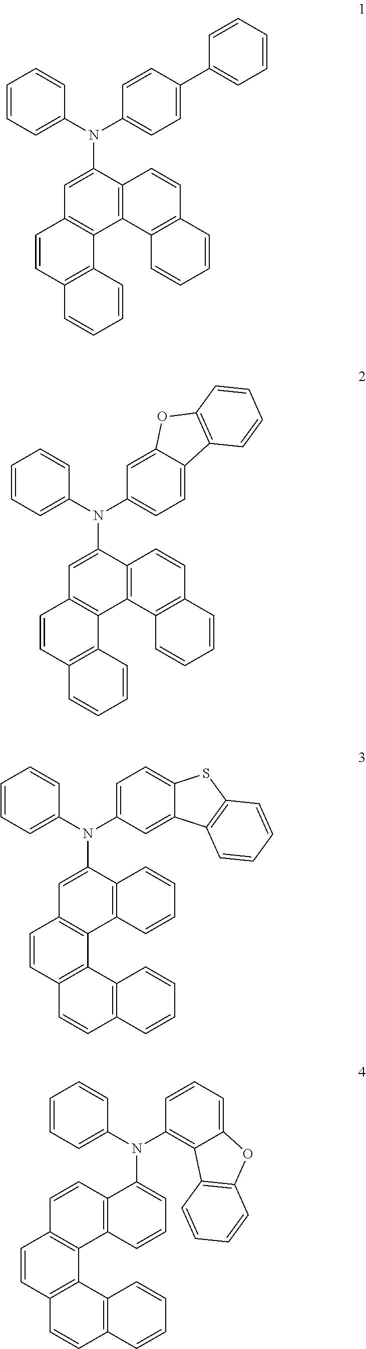 [SMILES: c1ccc(-c2ccc(N(c3ccccc3)c3cc4ccc5ccccc5c4c4c3ccc3ccccc34)cc2)cc1 ; c1ccc(N(c2ccc3c(c2)oc2ccccc23)c2cc3ccc4ccccc4c3c3c2ccc2ccccc23)cc1 ; c1ccc(N(c2ccc3sc4ccccc4c3c2)c2cc3ccc4ccc5ccccc5c4c3c3ccccc23)cc1 ; c1ccc(N(c2cccc3c2ccc2ccc4ccc5ccccc5c4c23)c2cccc3oc4ccccc4c23)cc1]

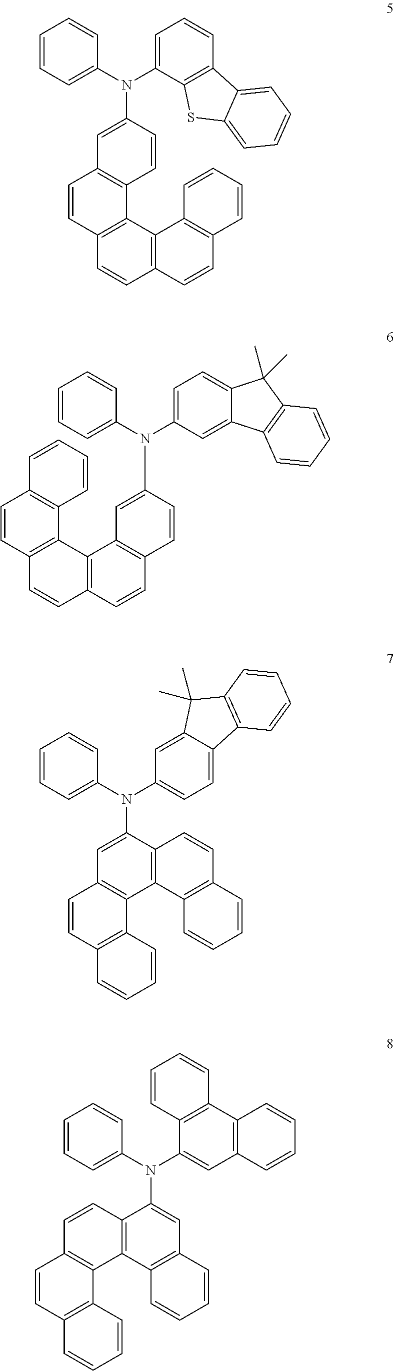 [SMILES: c1ccc(N(c2ccc3c(ccc4ccc5ccc6ccccc6c5c43)c2)c2cccc3c2sc2ccccc23)cc1 ; CC1(C)c2ccccc2-c2cc(N(c3ccccc3)c3ccc4ccc5ccc6ccc7ccccc7c6c5c4c3)ccc21 ; CC1(C)c2ccccc2-c2ccc(N(c3ccccc3)c3cc4ccc5ccccc5c4c4c3ccc3ccccc34)cc21 ; c1ccc(N(c2cc3ccccc3c3ccccc23)c2cc3ccccc3c3c2ccc2ccc4ccccc4c23)cc1]

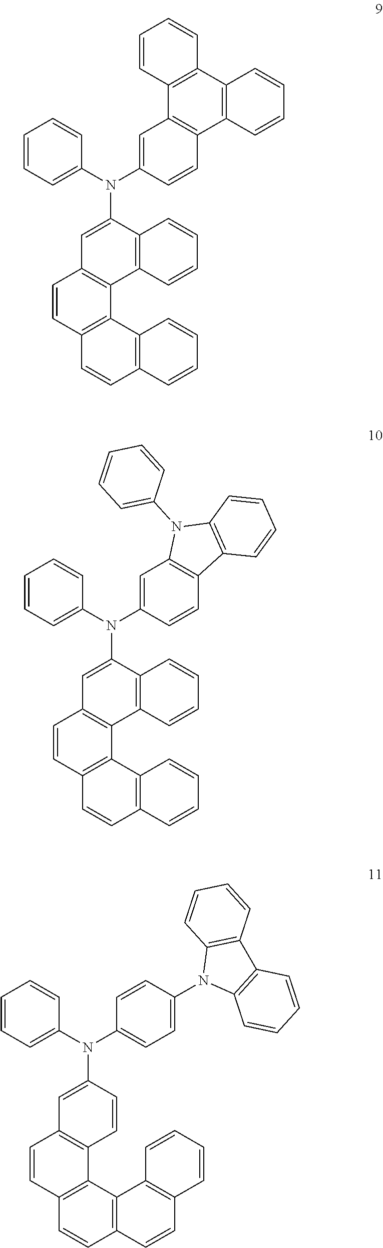 [SMILES: c1ccc(N(c2ccc3c4ccccc4c4ccccc4c3c2)c2cc3ccc4ccc5ccccc5c4c3c3ccccc23)cc1 ; c1ccc(N(c2ccc3c4ccccc4n(-c4ccccc4)c3c2)c2cc3ccc4ccc5ccccc5c4c3c3ccccc23)cc1 ; c1ccc(N(c2ccc(-n3c4ccccc4c4ccccc43)cc2)c2ccc3c(ccc4ccc5ccc6ccccc6c5c43)c2)cc1]

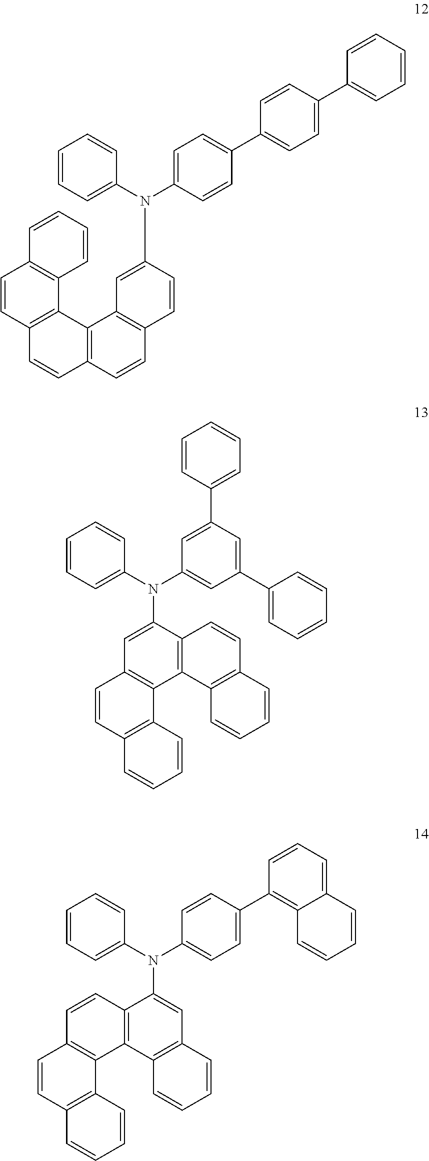 [SMILES: c1ccc(-c2ccc(-c3ccc(N(c4ccccc4)c4ccc5ccc6ccc7ccc8ccccc8c7c6c5c4)cc3)cc2)cc1 ; c1ccc(-c2cc(-c3ccccc3)cc(N(c3ccccc3)c3cc4ccc5ccccc5c4c4c3ccc3ccccc34)c2)cc1 ; c1ccc(N(c2ccc(-c3cccc4ccccc34)cc2)c2cc3ccccc3c3c2ccc2ccc4ccccc4c23)cc1]

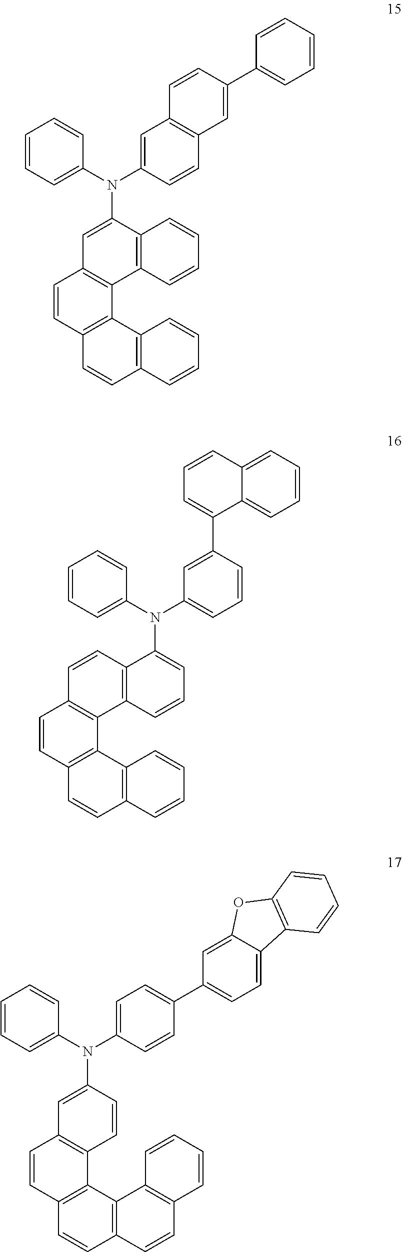 [SMILES: c1ccc(-c2ccc3cc(N(c4ccccc4)c4cc5ccc6ccc7ccccc7c6c5c5ccccc45)ccc3c2)cc1 ; c1ccc(N(c2cccc(-c3cccc4ccccc34)c2)c2cccc3c2ccc2ccc4ccc5ccccc5c4c23)cc1 ; c1ccc(N(c2ccc(-c3ccc4c(c3)oc3ccccc34)cc2)c2ccc3c(ccc4ccc5ccc6ccccc6c5c43)c2)cc1]

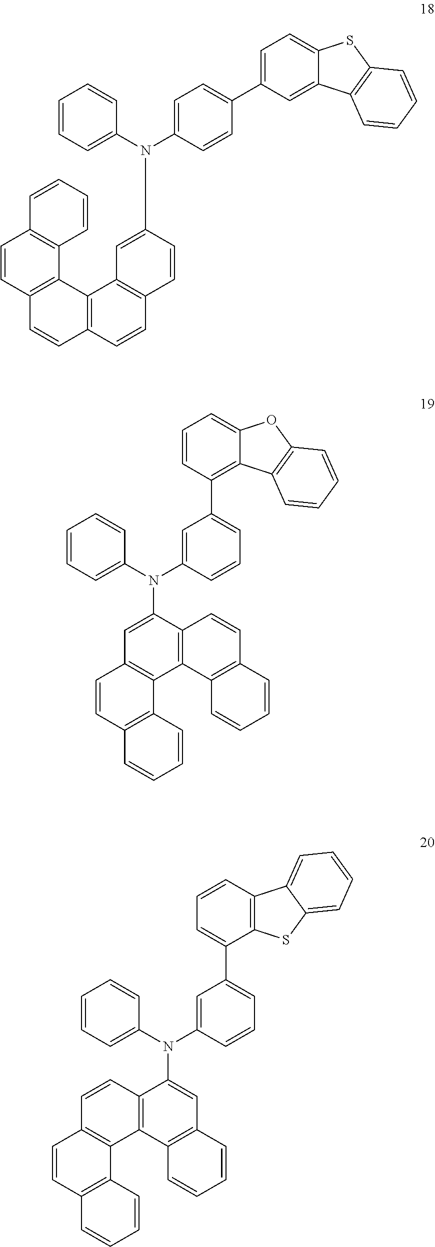 [SMILES: c1ccc(N(c2ccc(-c3ccc4sc5ccccc5c4c3)cc2)c2ccc3ccc4ccc5ccc6ccccc6c5c4c3c2)cc1 ; c1ccc(N(c2cccc(-c3cccc4oc5ccccc5c34)c2)c2cc3ccc4ccccc4c3c3c2ccc2ccccc23)cc1 ; c1ccc(N(c2cccc(-c3cccc4c3sc3ccccc34)c2)c2cc3ccccc3c3c2ccc2ccc4ccccc4c23)cc1]

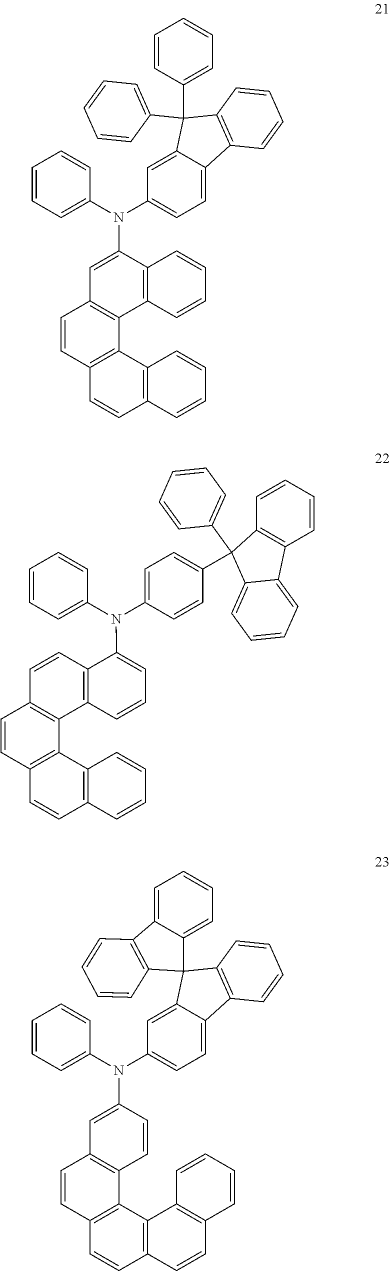 [SMILES: c1ccc(N(c2ccc3c(c2)C(c2ccccc2)(c2ccccc2)c2ccccc2-3)c2cc3ccc4ccc5ccccc5c4c3c3ccccc23)cc1 ; c1ccc(N(c2ccc(C3(c4ccccc4)c4ccccc4-c4ccccc43)cc2)c2cccc3c2ccc2ccc4ccc5ccccc5c4c23)cc1 ; c1ccc(N(c2ccc3c(c2)C2(c4ccccc4-c4ccccc42)c2ccccc2-3)c2ccc3c(ccc4ccc5ccc6ccccc6c5c43)c2)cc1]

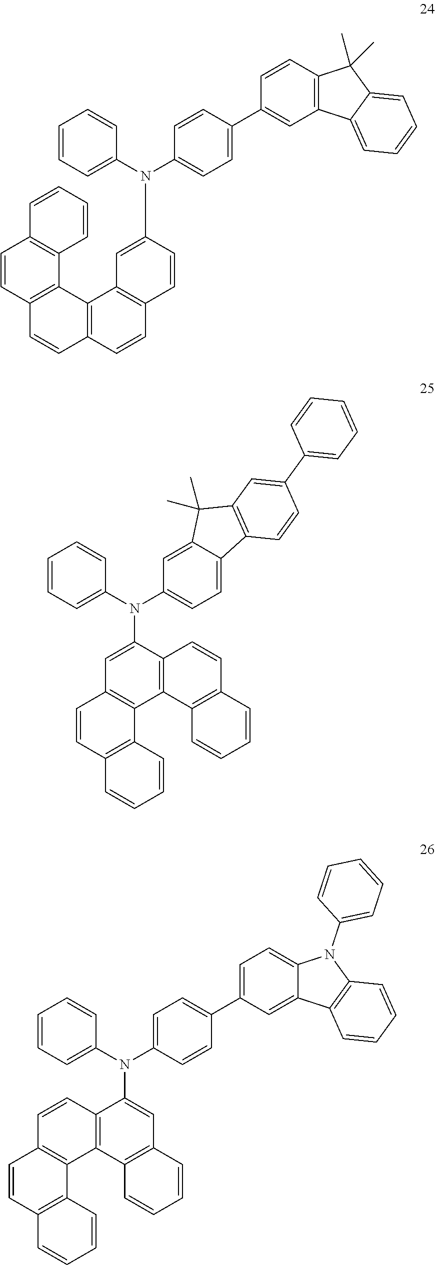 [SMILES: CC1(C)c2ccccc2-c2cc(-c3ccc(N(c4ccccc4)c4ccc5ccc6ccc7ccc8ccccc8c7c6c5c4)cc3)ccc21 ; CC1(C)c2cc(-c3ccccc3)ccc2-c2ccc(N(c3ccccc3)c3cc4ccc5ccccc5c4c4c3ccc3ccccc34)cc21 ; c1ccc(N(c2ccc(-c3ccc4c(c3)c3ccccc3n4-c3ccccc3)cc2)c2cc3ccccc3c3c2ccc2ccc4ccccc4c23)cc1]

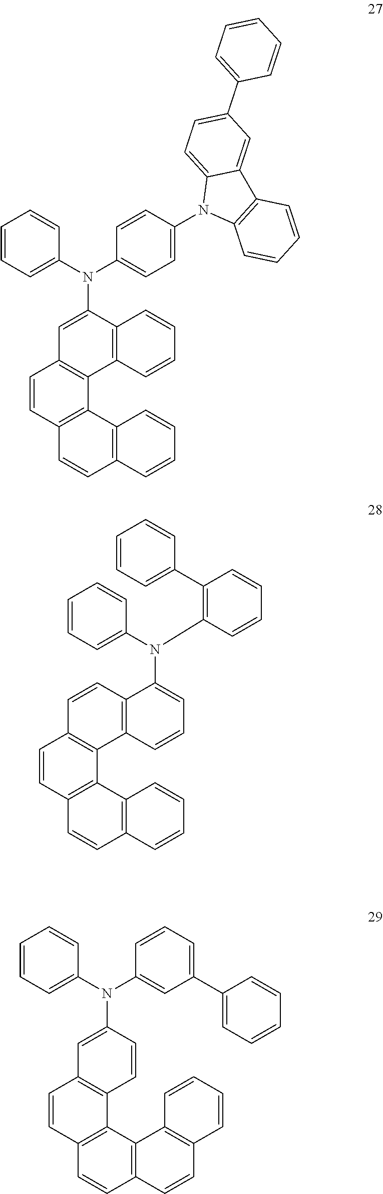

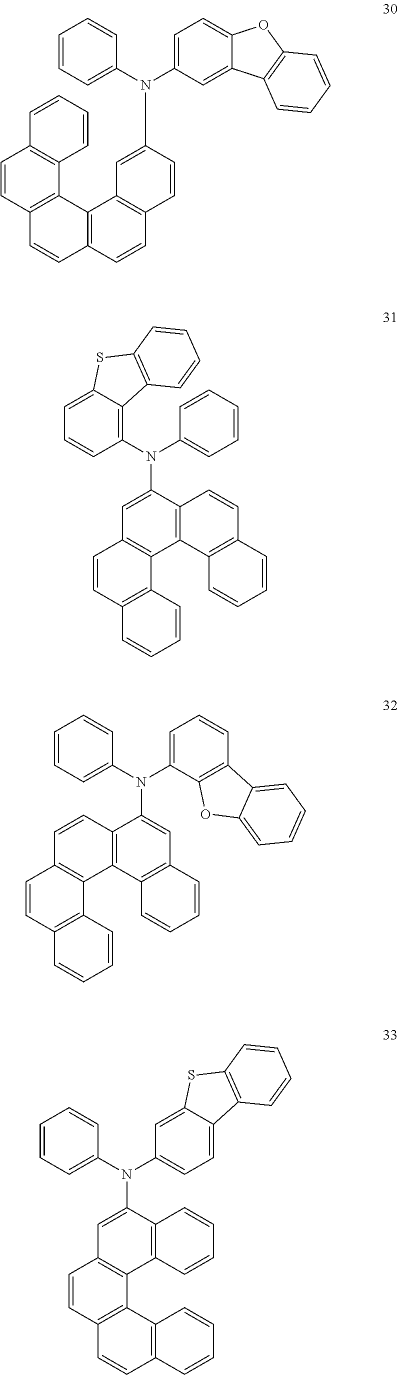 [SMILES: c1ccc(N(c2ccc3oc4ccccc4c3c2)c2ccc3ccc4ccc5ccc6ccccc6c5c4c3c2)cc1 ; c1ccc(N(c2cc3ccc4ccccc4c3c3c2ccc2ccccc23)c2cccc3sc4ccccc4c23)cc1 ; c1ccc(N(c2cc3ccccc3c3c2ccc2ccc4ccccc4c23)c2cccc3c2oc2ccccc23)cc1 ; c1ccc(N(c2ccc3c(c2)sc2ccccc23)c2cc3ccc4ccc5ccccc5c4c3c3ccccc23)cc1]

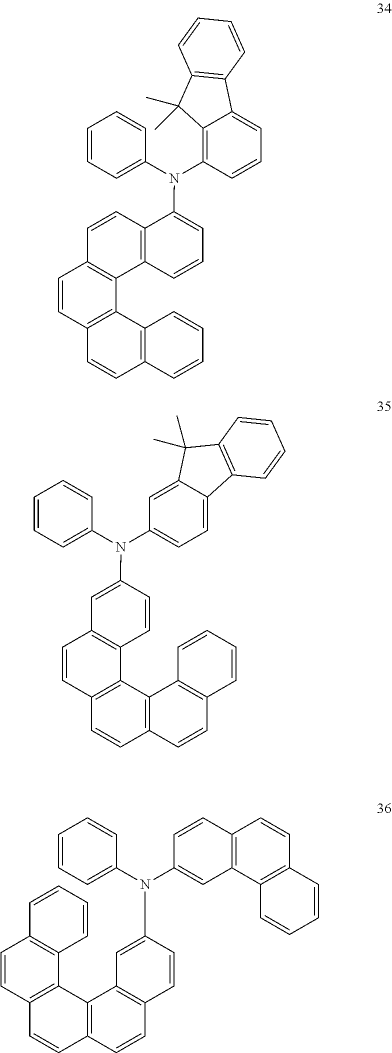

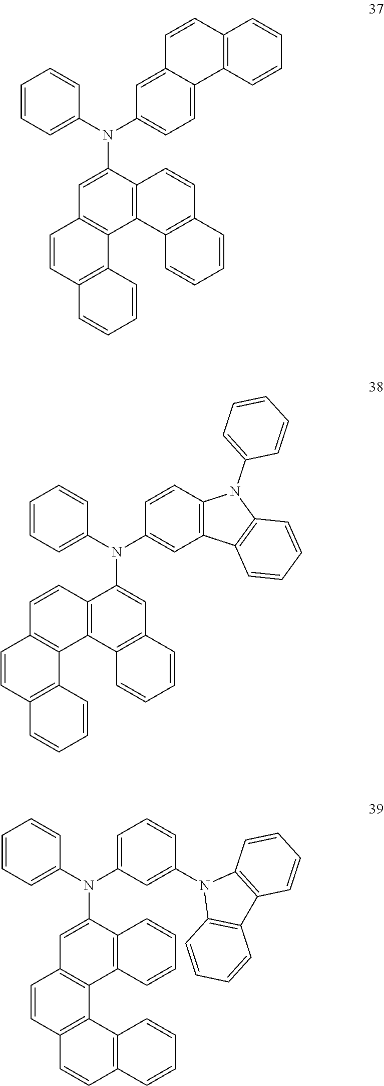 [SMILES: c1ccc(N(c2ccc3c(ccc4ccccc43)c2)c2cc3ccc4ccccc4c3c3c2ccc2ccccc23)cc1 ; c1ccc(N(c2ccc3c(c2)c2ccccc2n3-c2ccccc2)c2cc3ccccc3c3c2ccc2ccc4ccccc4c23)cc1 ; c1ccc(N(c2cccc(-n3c4ccccc4c4ccccc43)c2)c2cc3ccc4ccc5ccccc5c4c3c3ccccc23)cc1]

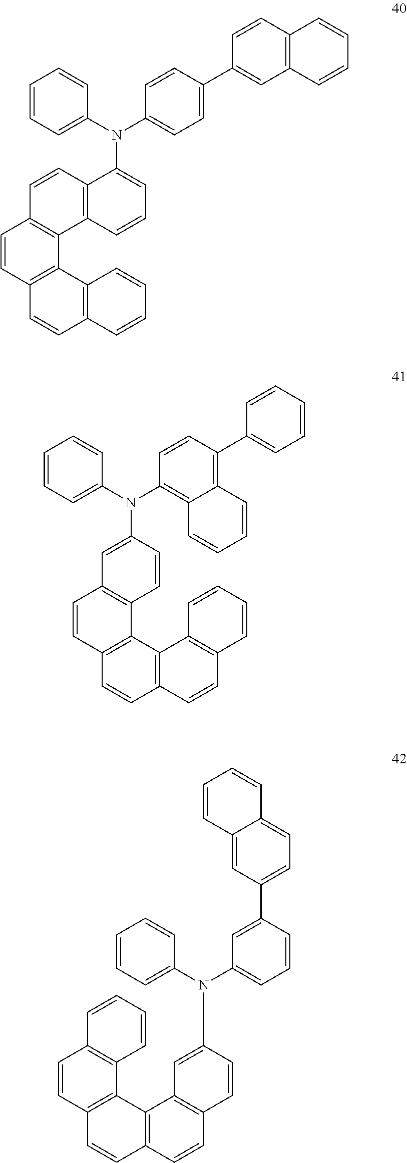 [SMILES: c1ccc(N(c2ccc(-c3ccc4ccccc4c3)cc2)c2cccc3c2ccc2ccc4ccc5ccccc5c4c23)cc1 ; c1ccc(-c2ccc(N(c3ccccc3)c3ccc4c(ccc5ccc6ccc7ccccc7c6c54)c3)c3ccccc23)cc1 ; c1ccc(N(c2cccc(-c3ccc4ccccc4c3)c2)c2ccc3ccc4ccc5ccc6ccccc6c5c4c3c2)cc1]

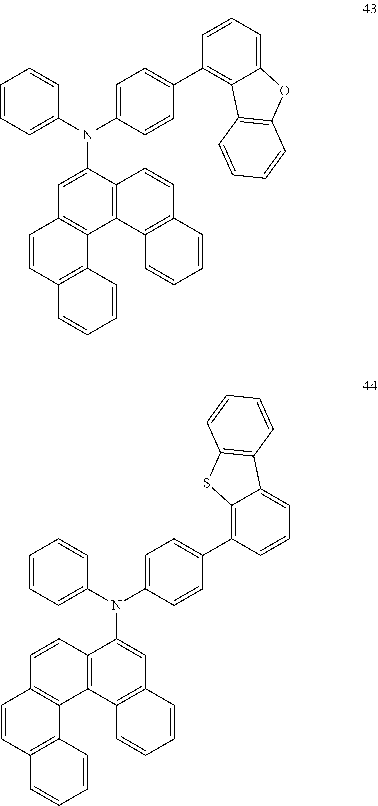 [SMILES: c1ccc(N(c2ccc(-c3cccc4oc5ccccc5c34)cc2)c2cc3ccc4ccccc4c3c3c2ccc2ccccc23)cc1 ; c1ccc(N(c2ccc(-c3cccc4c3sc3ccccc34)cc2)c2cc3ccccc3c3c2ccc2ccc4ccccc4c23)cc1]

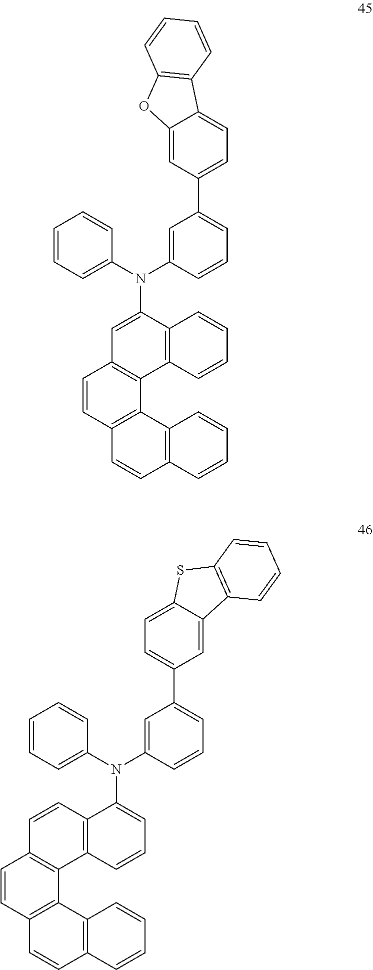 [SMILES: c1ccc(N(c2cccc(-c3ccc4c(c3)oc3ccccc34)c2)c2cc3ccc4ccc5ccccc5c4c3c3ccccc23)cc1 ; c1ccc(N(c2cccc(-c3ccc4sc5ccccc5c4c3)c2)c2cccc3c2ccc2ccc4ccc5ccccc5c4c23)cc1]

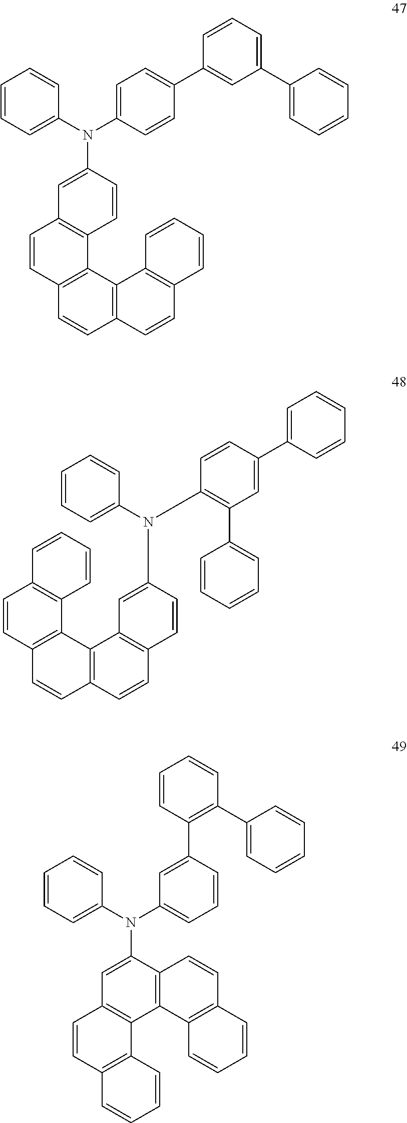 [SMILES: c1ccc(-c2cccc(-c3ccc(N(c4ccccc4)c4ccc5c(ccc6ccc7ccc8ccccc8c7c65)c4)cc3)c2)cc1 ; c1ccc(-c2ccc(N(c3ccccc3)c3ccc4ccc5ccc6ccc7ccccc7c6c5c4c3)c(-c3ccccc3)c2)cc1 ; c1ccc(-c2ccccc2-c2cccc(N(c3ccccc3)c3cc4ccc5ccccc5c4c4c3ccc3ccccc34)c2)cc1]

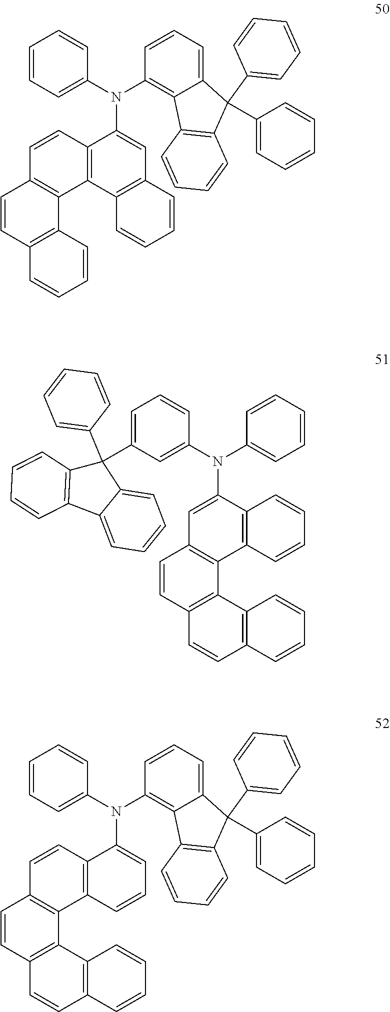 [SMILES: c1ccc(N(c2cccc3c2-c2ccccc2C3(c2ccccc2)c2ccccc2)c2cc3ccccc3c3c2ccc2ccc4ccccc4c23)cc1 ; c1ccc(N(c2cccc(C3(c4ccccc4)c4ccccc4-c4ccccc43)c2)c2cc3ccc4ccc5ccccc5c4c3c3ccccc23)cc1 ; c1ccc(N(c2cccc3c2-c2ccccc2C3(c2ccccc2)c2ccccc2)c2cccc3c2ccc2ccc4ccc5ccccc5c4c23)cc1]

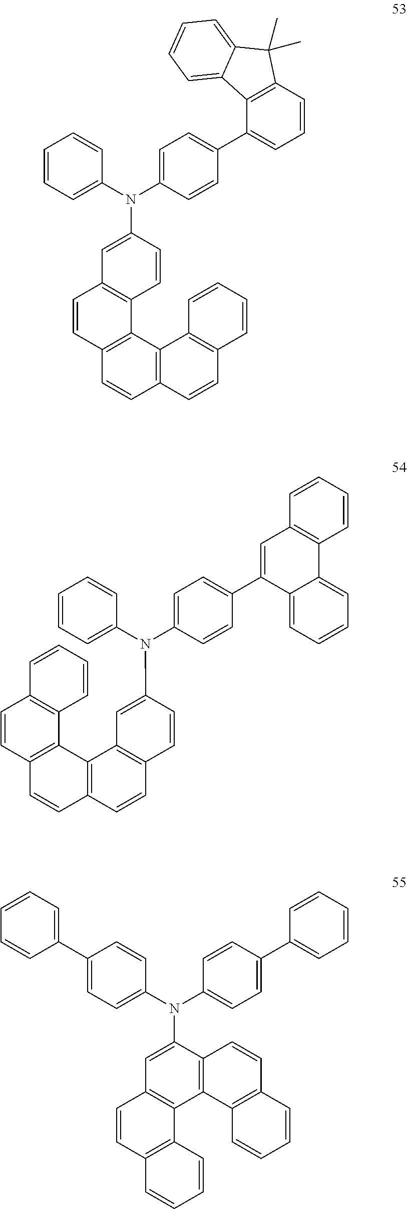 [SMILES: CC1(C)c2ccccc2-c2c(-c3ccc(N(c4ccccc4)c4ccc5c(ccc6ccc7ccc8ccccc8c7c65)c4)cc3)cccc21 ; c1ccc(N(c2ccc(-c3cc4ccccc4c4ccccc34)cc2)c2ccc3ccc4ccc5ccc6ccccc6c5c4c3c2)cc1 ; c1ccc(-c2ccc(N(c3ccc(-c4ccccc4)cc3)c3cc4ccc5ccccc5c4c4c3ccc3ccccc34)cc2)cc1]

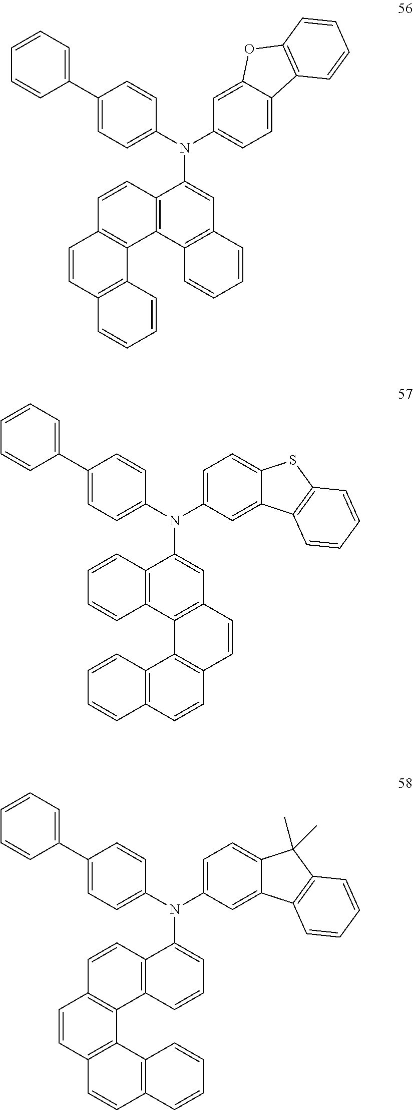 [SMILES: c1ccc(-c2ccc(N(c3ccc4c(c3)oc3ccccc34)c3cc4ccccc4c4c3ccc3ccc5ccccc5c34)cc2)cc1 ; c1ccc(-c2ccc(N(c3ccc4sc5ccccc5c4c3)c3cc4ccc5ccc6ccccc6c5c4c4ccccc34)cc2)cc1 ; CC1(C)c2ccccc2-c2cc(N(c3ccc(-c4ccccc4)cc3)c3cccc4c3ccc3ccc5ccc6ccccc6c5c34)ccc21]

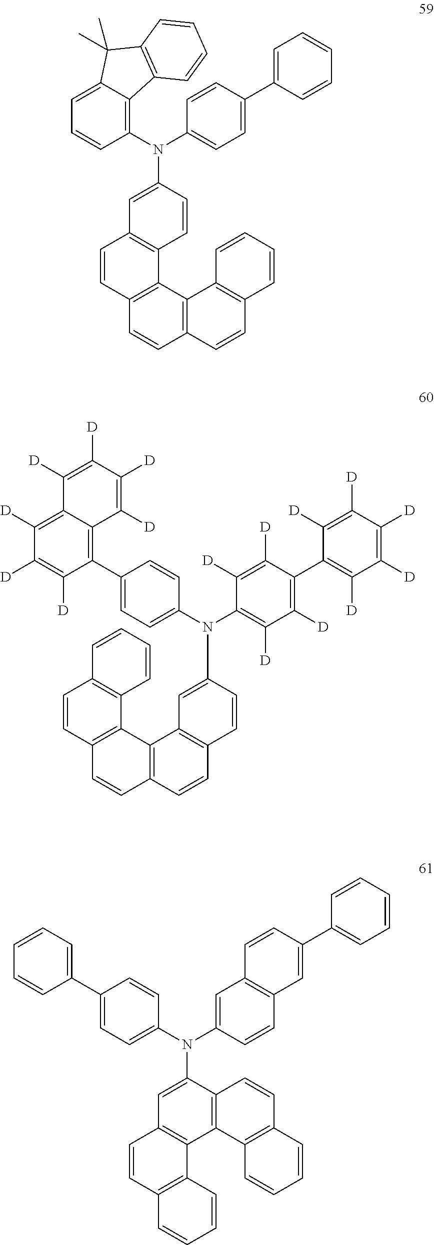 [SMILES: CC1(C)c2ccccc2-c2c(N(c3ccc(-c4ccccc4)cc3)c3ccc4c(ccc5ccc6ccc7ccccc7c6c54)c3)cccc21 ; [2H]c1c([2H])c([2H])c(-c2c([2H])c([2H])c(N(c3ccc(-c4c([2H])c([2H])c([2H])c5c([2H])c([2H])c([2H])c([2H])c45)cc3)c3ccc4ccc5ccc6ccc7ccccc7c6c5c4c3)c([2H])c2[2H])c([2H])c1[2H] ; c1ccc(-c2ccc(N(c3ccc4cc(-c5ccccc5)ccc4c3)c3cc4ccc5ccccc5c4c4c3ccc3ccccc34)cc2)cc1]

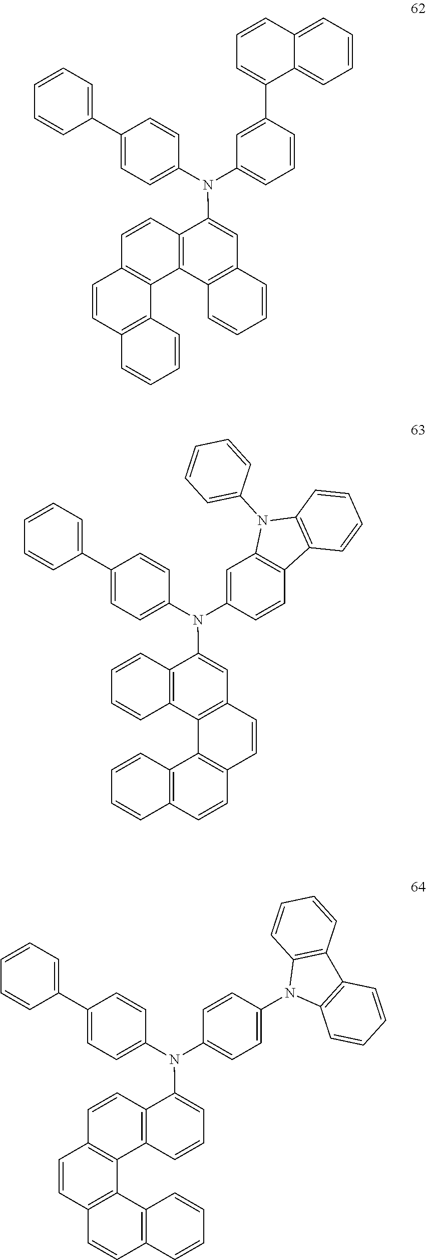 [SMILES: c1ccc(-c2ccc(N(c3cccc(-c4cccc5ccccc45)c3)c3cc4ccccc4c4c3ccc3ccc5ccccc5c34)cc2)cc1 ; c1ccc(-c2ccc(N(c3ccc4c5ccccc5n(-c5ccccc5)c4c3)c3cc4ccc5ccc6ccccc6c5c4c4ccccc34)cc2)cc1 ; c1ccc(-c2ccc(N(c3ccc(-n4c5ccccc5c5ccccc54)cc3)c3cccc4c3ccc3ccc5ccc6ccccc6c5c34)cc2)cc1]

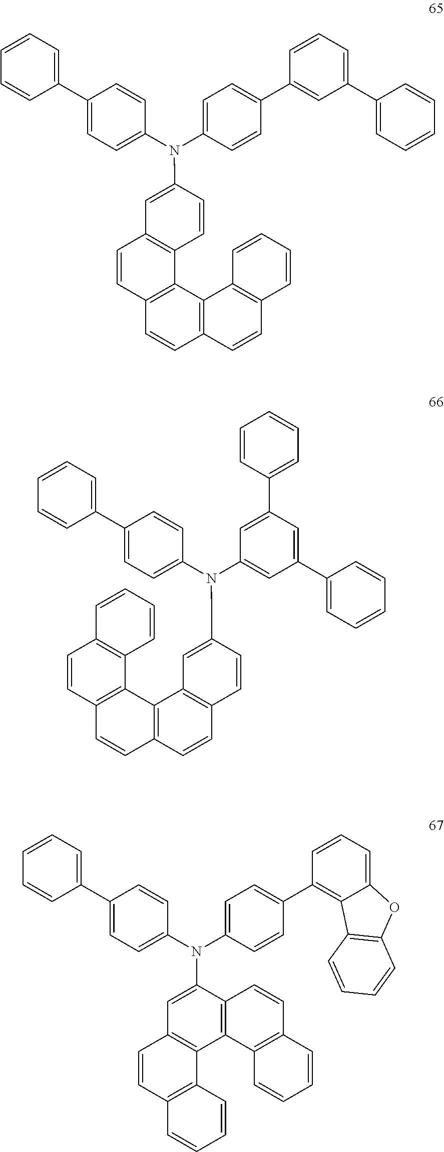 [SMILES: c1ccc(-c2ccc(N(c3ccc(-c4cccc(-c5ccccc5)c4)cc3)c3ccc4c(ccc5ccc6ccc7ccccc7c6c54)c3)cc2)cc1 ; c1ccc(-c2ccc(N(c3cc(-c4ccccc4)cc(-c4ccccc4)c3)c3ccc4ccc5ccc6ccc7ccccc7c6c5c4c3)cc2)cc1 ; c1ccc(-c2ccc(N(c3ccc(-c4cccc5oc6ccccc6c45)cc3)c3cc4ccc5ccccc5c4c4c3ccc3ccccc34)cc2)cc1]

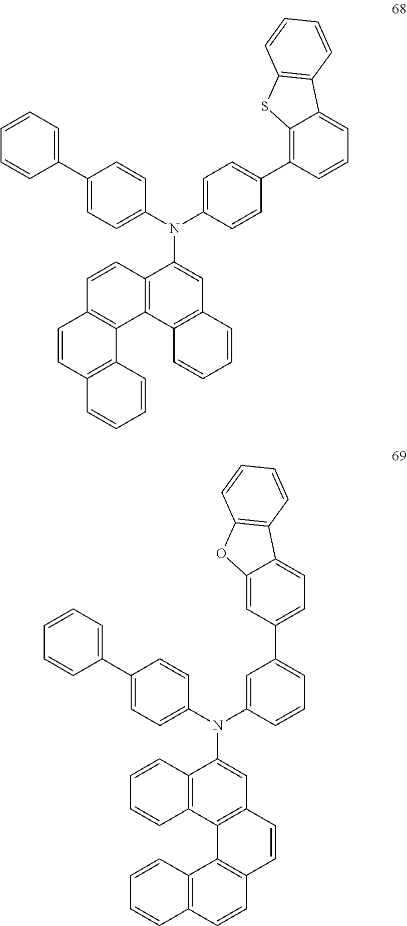 [SMILES: c1ccc(-c2ccc(N(c3ccc(-c4cccc5c4sc4ccccc45)cc3)c3cc4ccccc4c4c3ccc3ccc5ccccc5c34)cc2)cc1 ; c1ccc(-c2ccc(N(c3cccc(-c4ccc5c(c4)oc4ccccc45)c3)c3cc4ccc5ccc6ccccc6c5c4c4ccccc34)cc2)cc1]

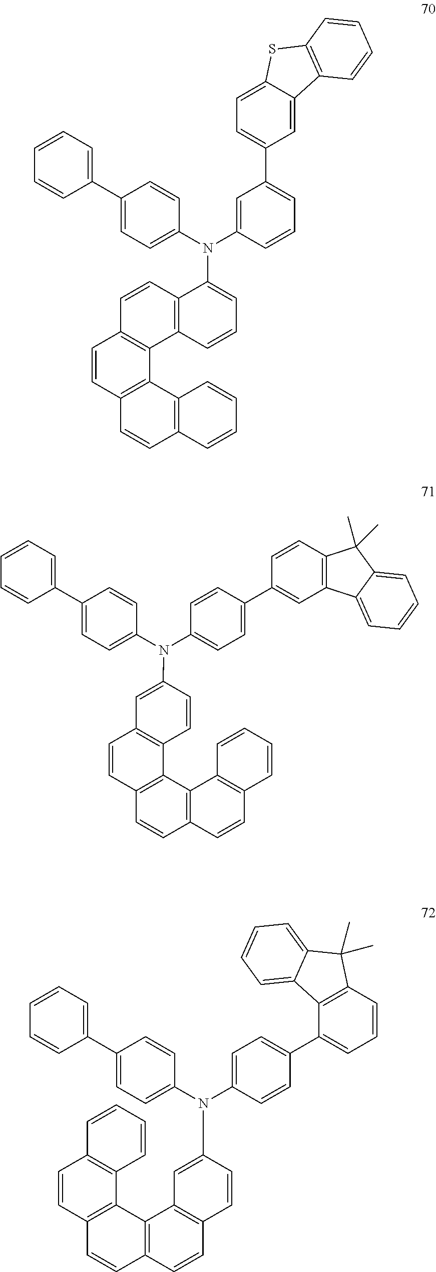 [SMILES: c1ccc(-c2ccc(N(c3cccc(-c4ccc5sc6ccccc6c5c4)c3)c3cccc4c3ccc3ccc5ccc6ccccc6c5c34)cc2)cc1 ; CC1(C)c2ccccc2-c2cc(-c3ccc(N(c4ccc(-c5ccccc5)cc4)c4ccc5c(ccc6ccc7ccc8ccccc8c7c65)c4)cc3)ccc21 ; CC1(C)c2ccccc2-c2c(-c3ccc(N(c4ccc(-c5ccccc5)cc4)c4ccc5ccc6ccc7ccc8ccccc8c7c6c5c4)cc3)cccc21]

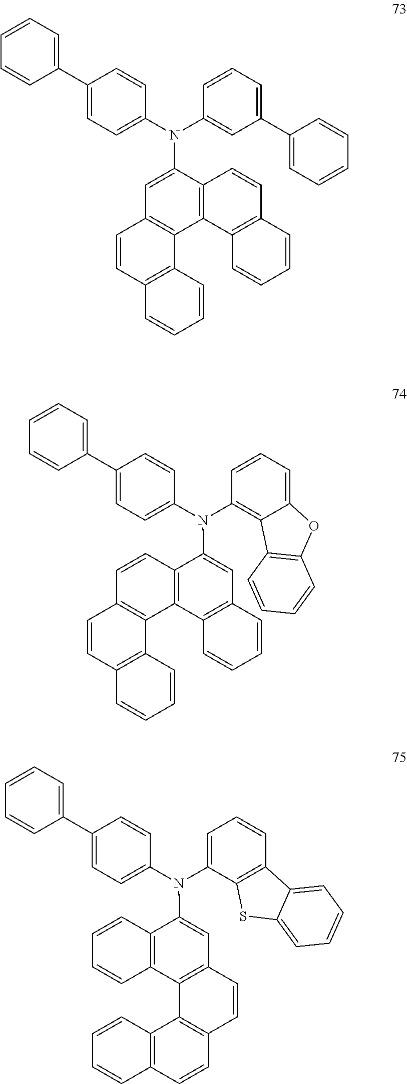 [SMILES: c1ccc(-c2ccc(N(c3cccc(-c4ccccc4)c3)c3cc4ccc5ccccc5c4c4c3ccc3ccccc34)cc2)cc1 ; c1ccc(-c2ccc(N(c3cc4ccccc4c4c3ccc3ccc5ccccc5c34)c3cccc4oc5ccccc5c34)cc2)cc1 ; c1ccc(-c2ccc(N(c3cc4ccc5ccc6ccccc6c5c4c4ccccc34)c3cccc4c3sc3ccccc34)cc2)cc1]

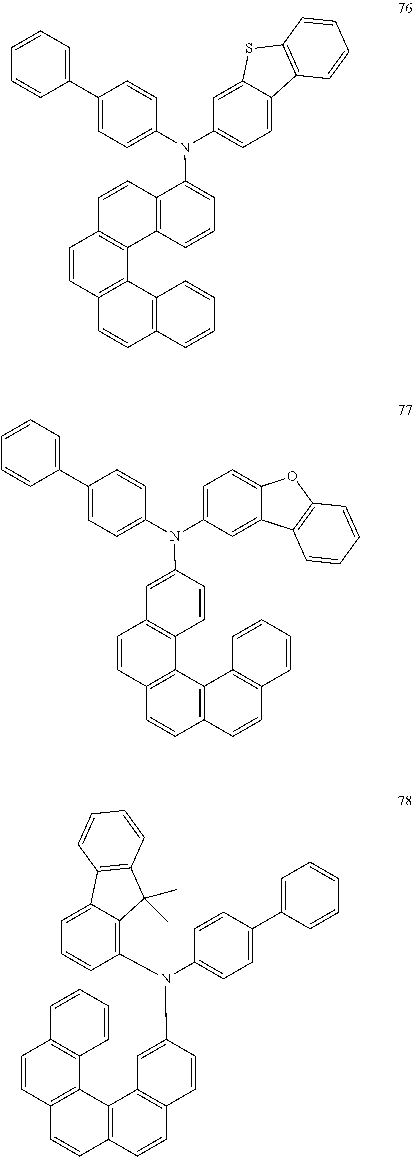 [SMILES: c1ccc(-c2ccc(N(c3ccc4c(c3)sc3ccccc34)c3cccc4c3ccc3ccc5ccc6ccccc6c5c34)cc2)cc1 ; c1ccc(-c2ccc(N(c3ccc4c(ccc5ccc6ccc7ccccc7c6c54)c3)c3ccc4oc5ccccc5c4c3)cc2)cc1 ; CC1(C)c2ccccc2-c2cccc(N(c3ccc(-c4ccccc4)cc3)c3ccc4ccc5ccc6ccc7ccccc7c6c5c4c3)c21]

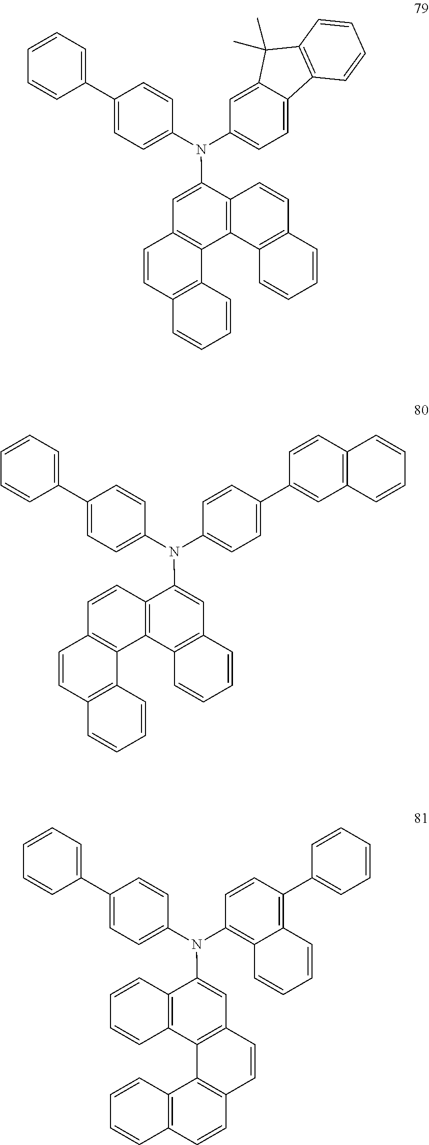 [SMILES: CC1(C)c2ccccc2-c2ccc(N(c3ccc(-c4ccccc4)cc3)c3cc4ccc5ccccc5c4c4c3ccc3ccccc34)cc21 ; c1ccc(-c2ccc(N(c3ccc(-c4ccc5ccccc5c4)cc3)c3cc4ccccc4c4c3ccc3ccc5ccccc5c34)cc2)cc1 ; c1ccc(-c2ccc(N(c3ccc(-c4ccccc4)c4ccccc34)c3cc4ccc5ccc6ccccc6c5c4c4ccccc34)cc2)cc1]

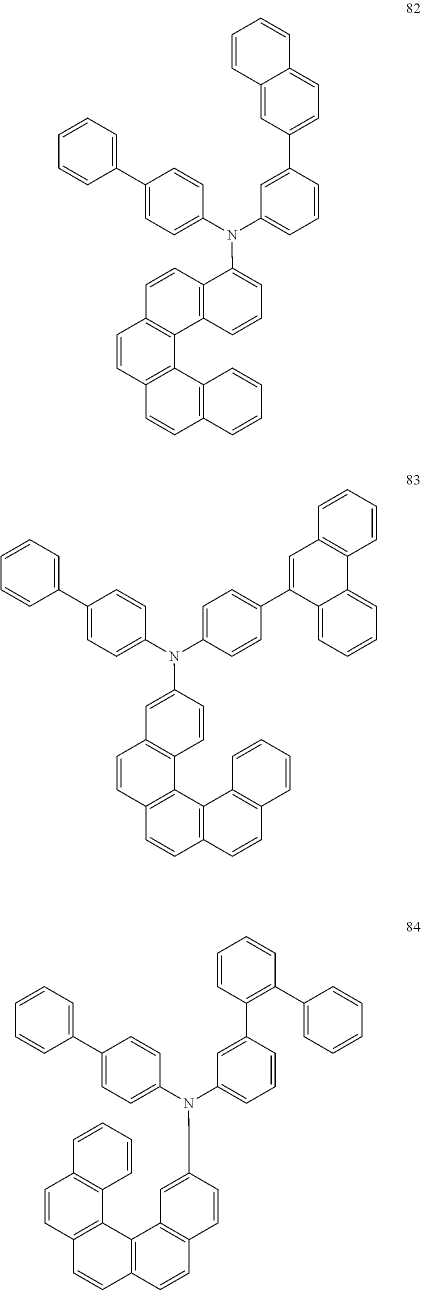 [SMILES: c1ccc(-c2ccc(N(c3cccc(-c4ccc5ccccc5c4)c3)c3cccc4c3ccc3ccc5ccc6ccccc6c5c34)cc2)cc1 ; c1ccc(-c2ccc(N(c3ccc(-c4cc5ccccc5c5ccccc45)cc3)c3ccc4c(ccc5ccc6ccc7ccccc7c6c54)c3)cc2)cc1 ; c1ccc(-c2ccc(N(c3cccc(-c4ccccc4-c4ccccc4)c3)c3ccc4ccc5ccc6ccc7ccccc7c6c5c4c3)cc2)cc1]

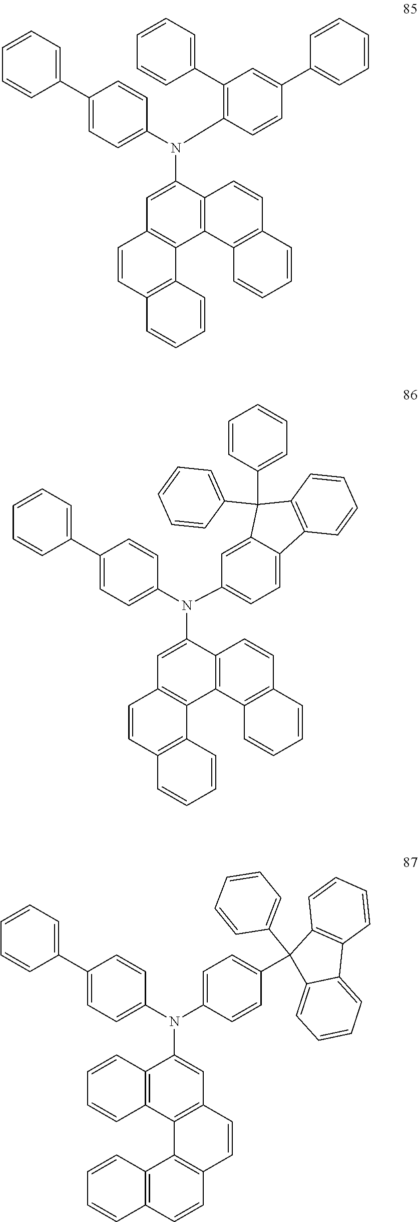 [SMILES: c1ccc(-c2ccc(N(c3ccc(-c4ccccc4)cc3-c3ccccc3)c3cc4ccc5ccccc5c4c4c3ccc3ccccc34)cc2)cc1 ; c1ccc(-c2ccc(N(c3ccc4c(c3)C(c3ccccc3)(c3ccccc3)c3ccccc3-4)c3cc4ccc5ccccc5c4c4c3ccc3ccccc34)cc2)cc1 ; c1ccc(-c2ccc(N(c3ccc(C4(c5ccccc5)c5ccccc5-c5ccccc54)cc3)c3cc4ccc5ccc6ccccc6c5c4c4ccccc34)cc2)cc1]

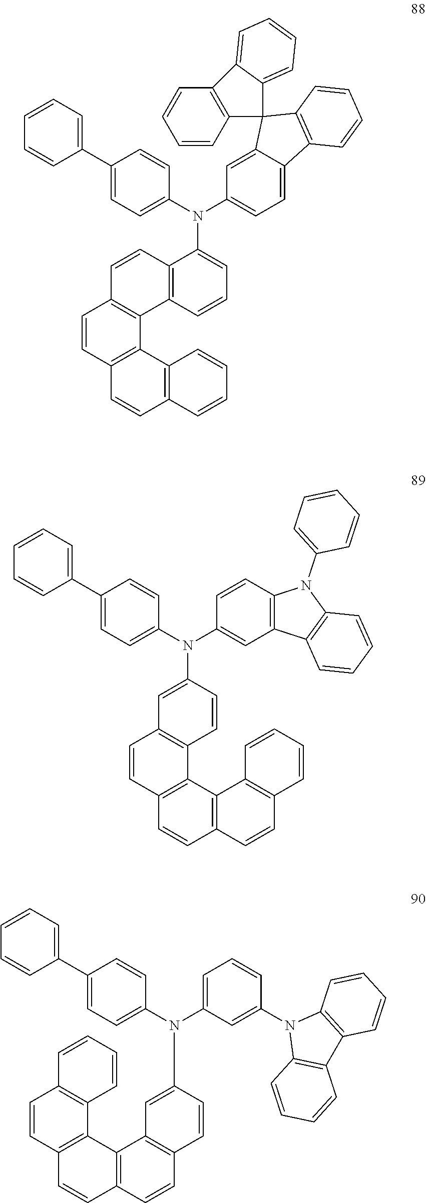 [SMILES: c1ccc(-c2ccc(N(c3ccc4c(c3)C3(c5ccccc5-c5ccccc53)c3ccccc3-4)c3cccc4c3ccc3ccc5ccc6ccccc6c5c34)cc2)cc1 ; c1ccc(-c2ccc(N(c3ccc4c(ccc5ccc6ccc7ccccc7c6c54)c3)c3ccc4c(c3)c3ccccc3n4-c3ccccc3)cc2)cc1 ; c1ccc(-c2ccc(N(c3cccc(-n4c5ccccc5c5ccccc54)c3)c3ccc4ccc5ccc6ccc7ccccc7c6c5c4c3)cc2)cc1]

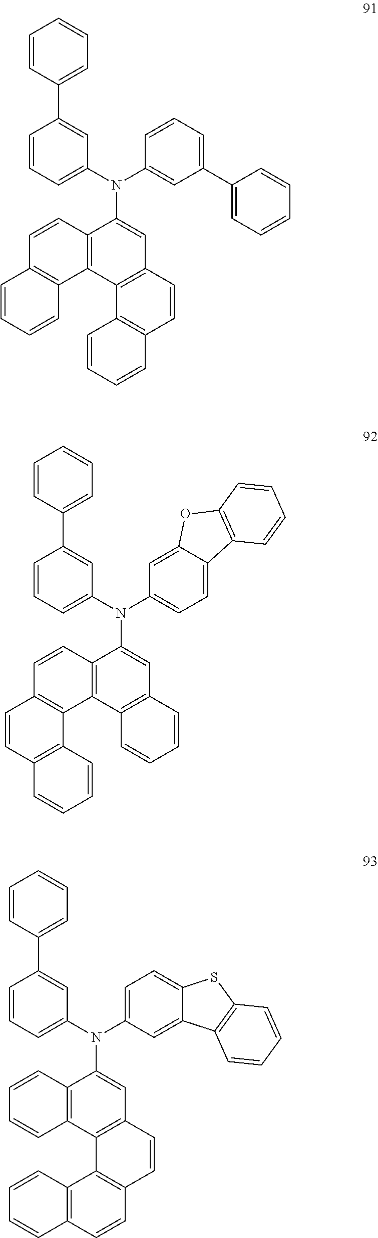 [SMILES: c1ccc(-c2cccc(N(c3cccc(-c4ccccc4)c3)c3cc4ccc5ccccc5c4c4c3ccc3ccccc34)c2)cc1 ; c1ccc(-c2cccc(N(c3ccc4c(c3)oc3ccccc34)c3cc4ccccc4c4c3ccc3ccc5ccccc5c34)c2)cc1 ; c1ccc(-c2cccc(N(c3ccc4sc5ccccc5c4c3)c3cc4ccc5ccc6ccccc6c5c4c4ccccc34)c2)cc1]

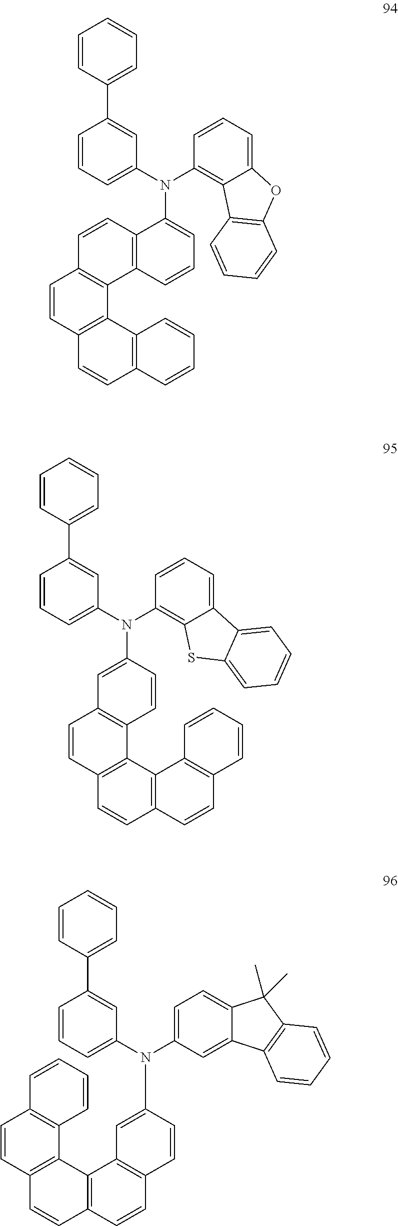 [SMILES: c1ccc(-c2cccc(N(c3cccc4c3ccc3ccc5ccc6ccccc6c5c34)c3cccc4oc5ccccc5c34)c2)cc1 ; c1ccc(-c2cccc(N(c3ccc4c(ccc5ccc6ccc7ccccc7c6c54)c3)c3cccc4c3sc3ccccc34)c2)cc1 ; CC1(C)c2ccccc2-c2cc(N(c3cccc(-c4ccccc4)c3)c3ccc4ccc5ccc6ccc7ccccc7c6c5c4c3)ccc21]

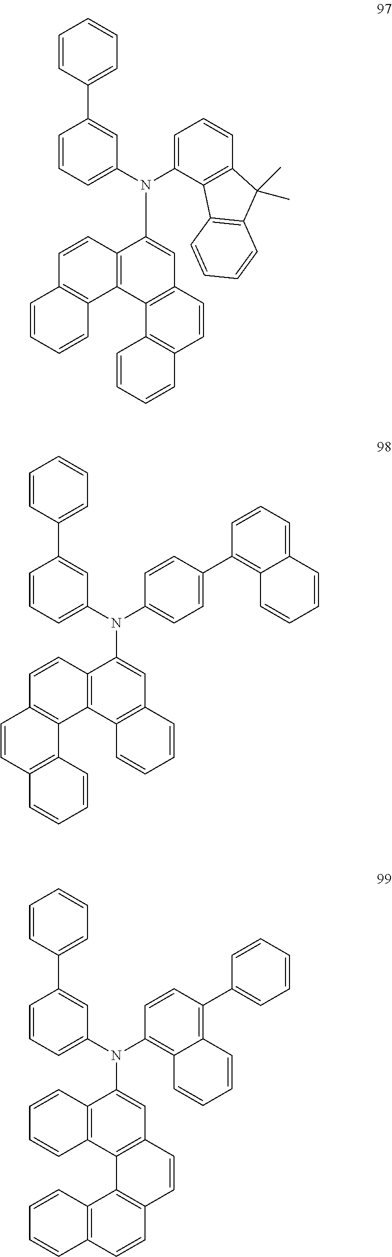 [SMILES: CC1(C)c2ccccc2-c2c(N(c3cccc(-c4ccccc4)c3)c3cc4ccc5ccccc5c4c4c3ccc3ccccc34)cccc21 ; c1ccc(-c2cccc(N(c3ccc(-c4cccc5ccccc45)cc3)c3cc4ccccc4c4c3ccc3ccc5ccccc5c34)c2)cc1 ; c1ccc(-c2cccc(N(c3ccc(-c4ccccc4)c4ccccc34)c3cc4ccc5ccc6ccccc6c5c4c4ccccc34)c2)cc1]

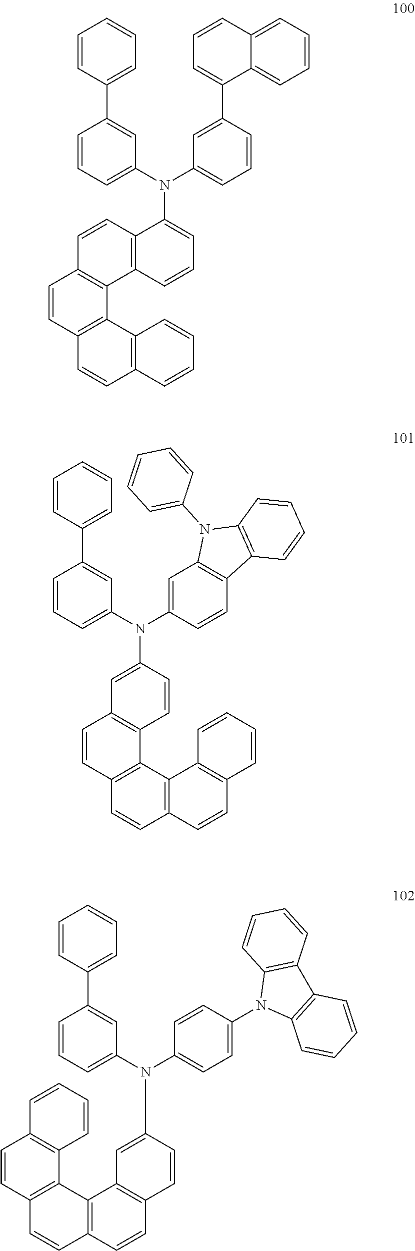 [SMILES: c1ccc(-c2cccc(N(c3cccc(-c4cccc5ccccc45)c3)c3cccc4c3ccc3ccc5ccc6ccccc6c5c34)c2)cc1 ; c1ccc(-c2cccc(N(c3ccc4c(ccc5ccc6ccc7ccccc7c6c54)c3)c3ccc4c5ccccc5n(-c5ccccc5)c4c3)c2)cc1 ; c1ccc(-c2cccc(N(c3ccc(-n4c5ccccc5c5ccccc54)cc3)c3ccc4ccc5ccc6ccc7ccccc7c6c5c4c3)c2)cc1]

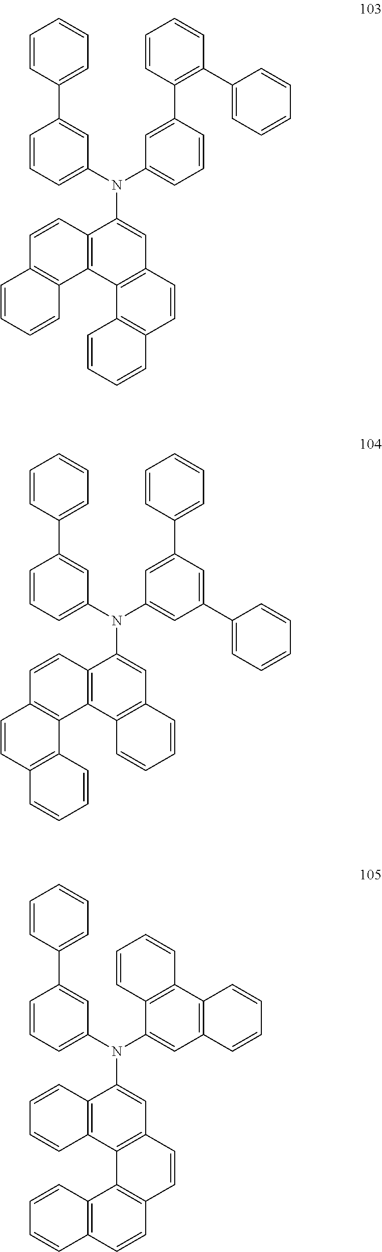 [SMILES: c1ccc(-c2cccc(N(c3cccc(-c4ccccc4-c4ccccc4)c3)c3cc4ccc5ccccc5c4c4c3ccc3ccccc34)c2)cc1 ; c1ccc(-c2cccc(N(c3cc(-c4ccccc4)cc(-c4ccccc4)c3)c3cc4ccccc4c4c3ccc3ccc5ccccc5c34)c2)cc1 ; c1ccc(-c2cccc(N(c3cc4ccccc4c4ccccc34)c3cc4ccc5ccc6ccccc6c5c4c4ccccc34)c2)cc1]

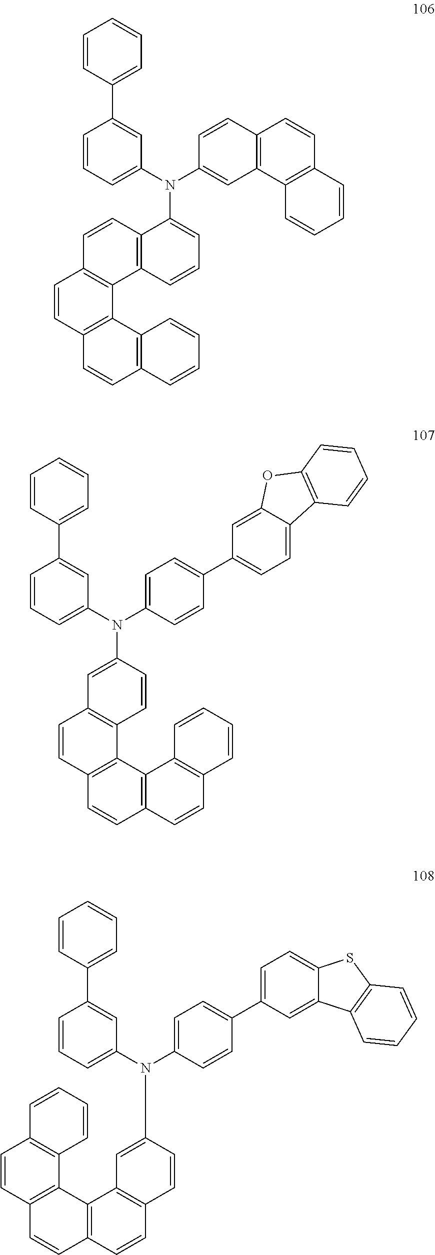 [SMILES: c1ccc(-c2cccc(N(c3ccc4ccc5ccccc5c4c3)c3cccc4c3ccc3ccc5ccc6ccccc6c5c34)c2)cc1 ; c1ccc(-c2cccc(N(c3ccc(-c4ccc5c(c4)oc4ccccc45)cc3)c3ccc4c(ccc5ccc6ccc7ccccc7c6c54)c3)c2)cc1 ; c1ccc(-c2cccc(N(c3ccc(-c4ccc5sc6ccccc6c5c4)cc3)c3ccc4ccc5ccc6ccc7ccccc7c6c5c4c3)c2)cc1]

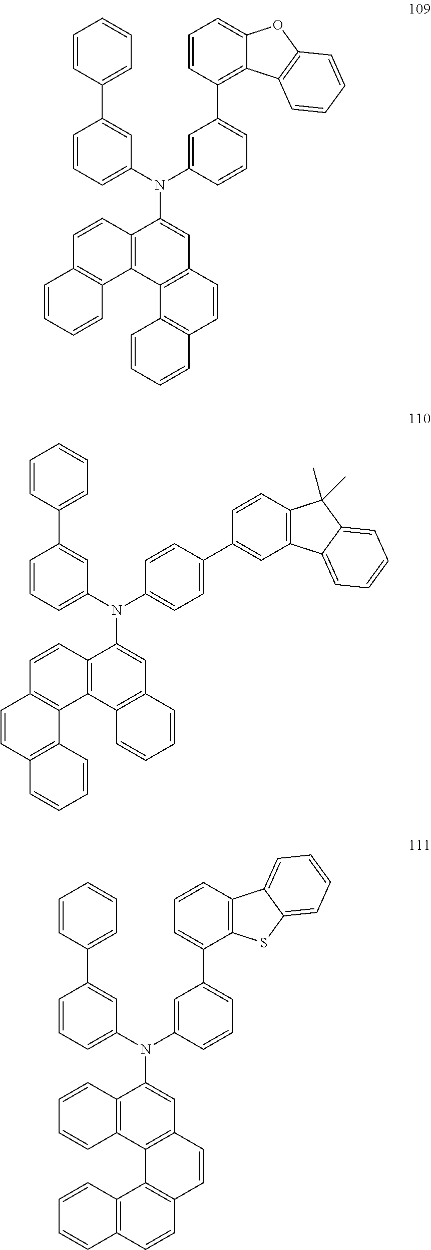 [SMILES: c1ccc(-c2cccc(N(c3cccc(-c4cccc5oc6ccccc6c45)c3)c3cc4ccc5ccccc5c4c4c3ccc3ccccc34)c2)cc1 ; CC1(C)c2ccccc2-c2cc(-c3ccc(N(c4cccc(-c5ccccc5)c4)c4cc5ccccc5c5c4ccc4ccc6ccccc6c45)cc3)ccc21 ; c1ccc(-c2cccc(N(c3cccc(-c4cccc5c4sc4ccccc45)c3)c3cc4ccc5ccc6ccccc6c5c4c4ccccc34)c2)cc1]

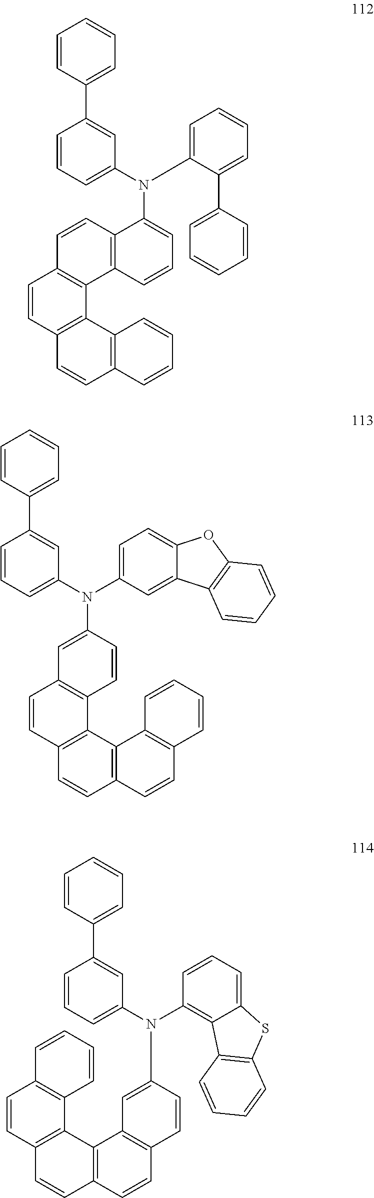 [SMILES: c1ccc(-c2cccc(N(c3ccccc3-c3ccccc3)c3cccc4c3ccc3ccc5ccc6ccccc6c5c34)c2)cc1 ; c1ccc(-c2cccc(N(c3ccc4c(ccc5ccc6ccc7ccccc7c6c54)c3)c3ccc4oc5ccccc5c4c3)c2)cc1 ; c1ccc(-c2cccc(N(c3ccc4ccc5ccc6ccc7ccccc7c6c5c4c3)c3cccc4sc5ccccc5c34)c2)cc1]

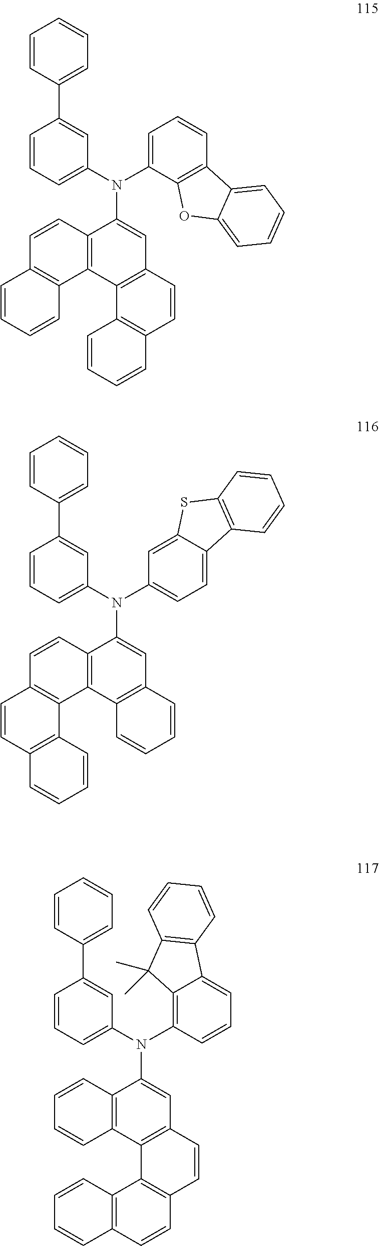 [SMILES: c1ccc(-c2cccc(N(c3cc4ccc5ccccc5c4c4c3ccc3ccccc34)c3cccc4c3oc3ccccc34)c2)cc1 ; c1ccc(-c2cccc(N(c3ccc4c(c3)sc3ccccc34)c3cc4ccccc4c4c3ccc3ccc5ccccc5c34)c2)cc1 ; CC1(C)c2ccccc2-c2cccc(N(c3cccc(-c4ccccc4)c3)c3cc4ccc5ccc6ccccc6c5c4c4ccccc34)c21]

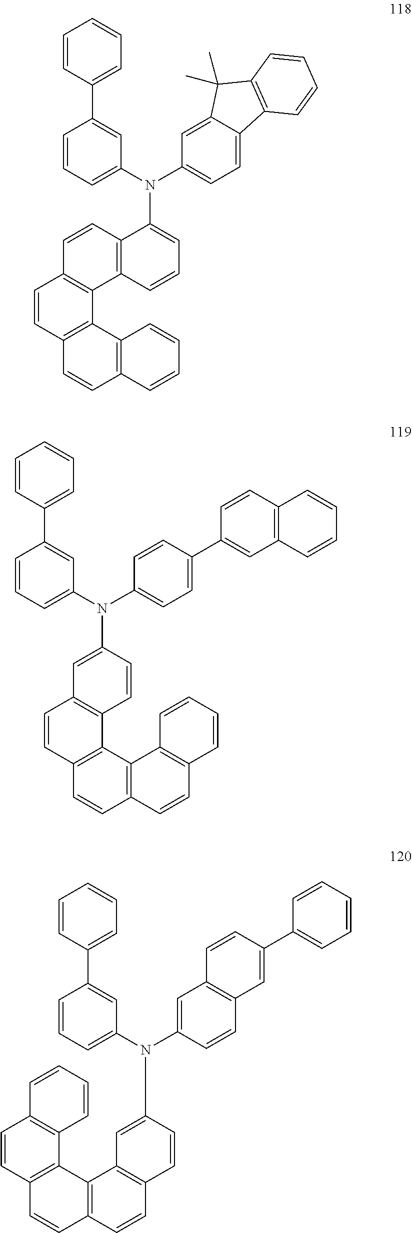 [SMILES: CC1(C)c2ccccc2-c2ccc(N(c3cccc(-c4ccccc4)c3)c3cccc4c3ccc3ccc5ccc6ccccc6c5c34)cc21 ; c1ccc(-c2cccc(N(c3ccc(-c4ccc5ccccc5c4)cc3)c3ccc4c(ccc5ccc6ccc7ccccc7c6c54)c3)c2)cc1 ; c1ccc(-c2cccc(N(c3ccc4cc(-c5ccccc5)ccc4c3)c3ccc4ccc5ccc6ccc7ccccc7c6c5c4c3)c2)cc1]

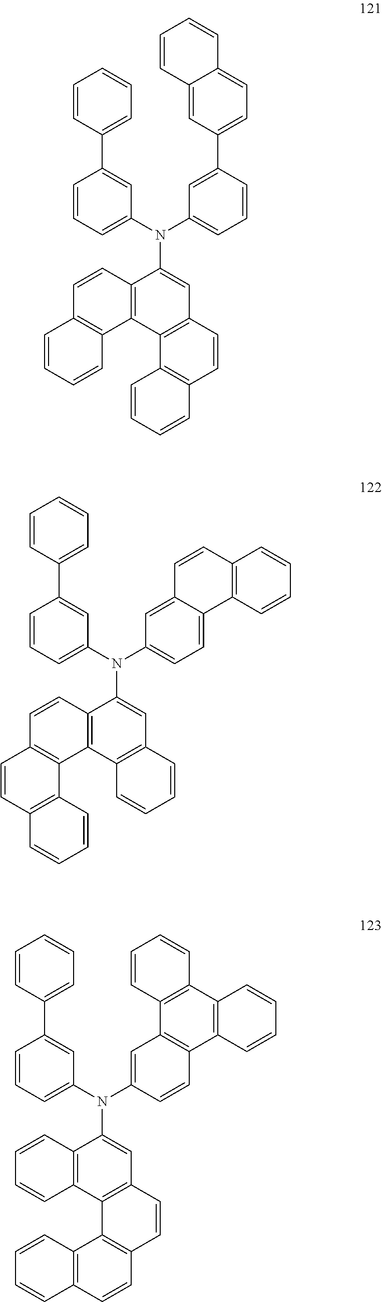 [SMILES: c1ccc(-c2cccc(N(c3cccc(-c4ccc5ccccc5c4)c3)c3cc4ccc5ccccc5c4c4c3ccc3ccccc34)c2)cc1 ; c1ccc(-c2cccc(N(c3ccc4c(ccc5ccccc54)c3)c3cc4ccccc4c4c3ccc3ccc5ccccc5c34)c2)cc1 ; c1ccc(-c2cccc(N(c3ccc4c5ccccc5c5ccccc5c4c3)c3cc4ccc5ccc6ccccc6c5c4c4ccccc34)c2)cc1]

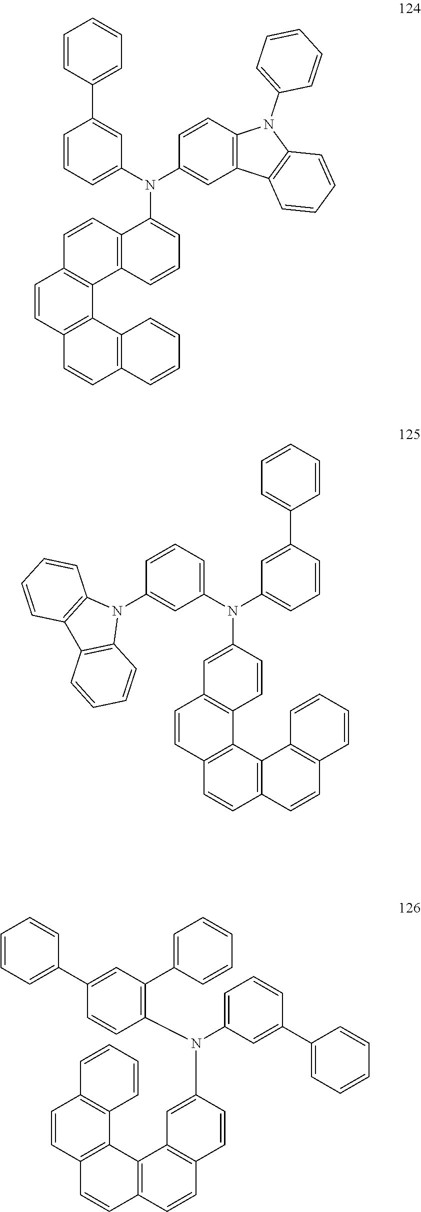 [SMILES: c1ccc(-c2cccc(N(c3ccc4c(c3)c3ccccc3n4-c3ccccc3)c3cccc4c3ccc3ccc5ccc6ccccc6c5c34)c2)cc1 ; c1ccc(-c2cccc(N(c3cccc(-n4c5ccccc5c5ccccc54)c3)c3ccc4c(ccc5ccc6ccc7ccccc7c6c54)c3)c2)cc1 ; c1ccc(-c2cccc(N(c3ccc4ccc5ccc6ccc7ccccc7c6c5c4c3)c3ccc(-c4ccccc4)cc3-c3ccccc3)c2)cc1]

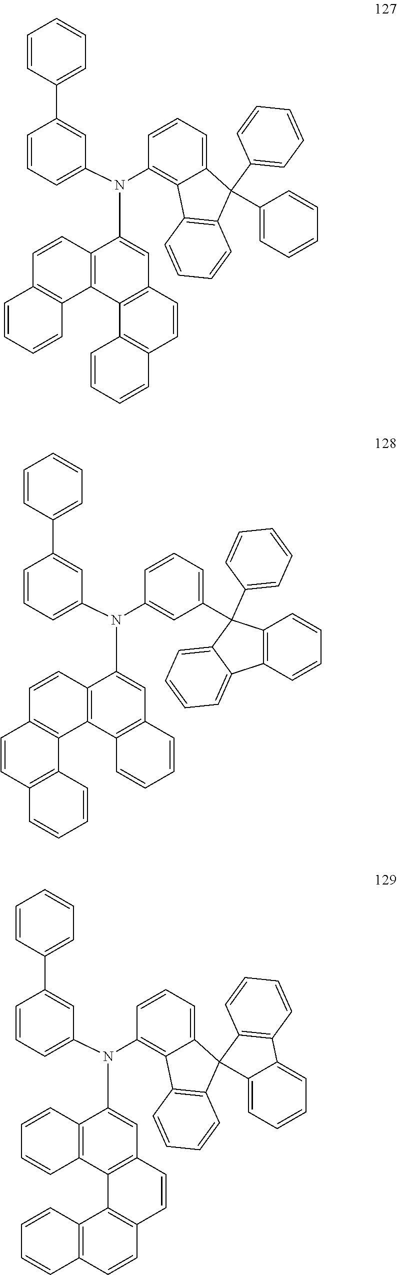 [SMILES: c1ccc(-c2cccc(N(c3cccc4c3-c3ccccc3C4(c3ccccc3)c3ccccc3)c3cc4ccc5ccccc5c4c4c3ccc3ccccc34)c2)cc1 ; c1ccc(-c2cccc(N(c3cccc(C4(c5ccccc5)c5ccccc5-c5ccccc54)c3)c3cc4ccccc4c4c3ccc3ccc5ccccc5c34)c2)cc1 ; c1ccc(-c2cccc(N(c3cccc4c3-c3ccccc3C43c4ccccc4-c4ccccc43)c3cc4ccc5ccc6ccccc6c5c4c4ccccc34)c2)cc1]

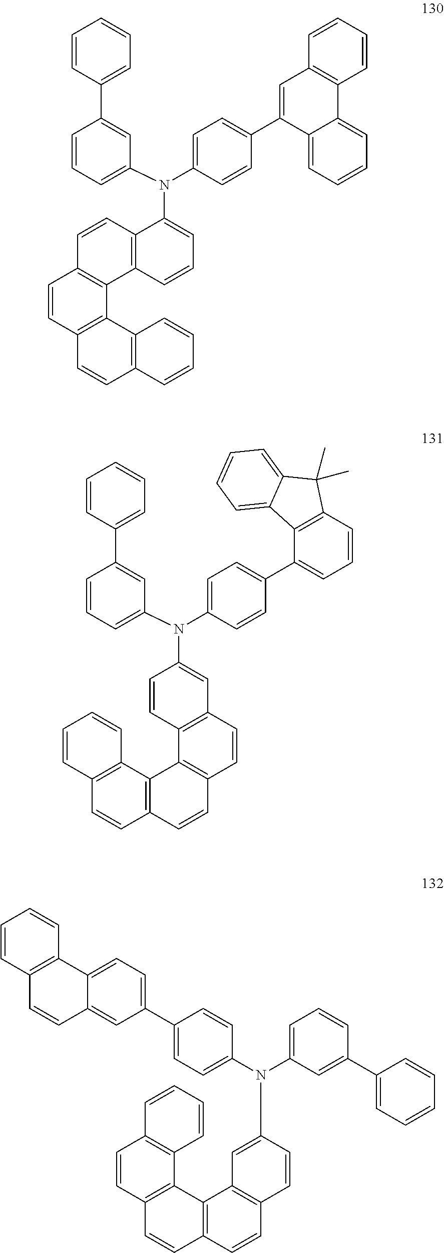 [SMILES: c1ccc(-c2cccc(N(c3ccc(-c4cc5ccccc5c5ccccc45)cc3)c3cccc4c3ccc3ccc5ccc6ccccc6c5c34)c2)cc1 ; CC1(C)c2ccccc2-c2c(-c3ccc(N(c4cccc(-c5ccccc5)c4)c4ccc5c(ccc6ccc7ccc8ccccc8c7c65)c4)cc3)cccc21 ; c1ccc(-c2cccc(N(c3ccc(-c4ccc5c(ccc6ccccc65)c4)cc3)c3ccc4ccc5ccc6ccc7ccccc7c6c5c4c3)c2)cc1]

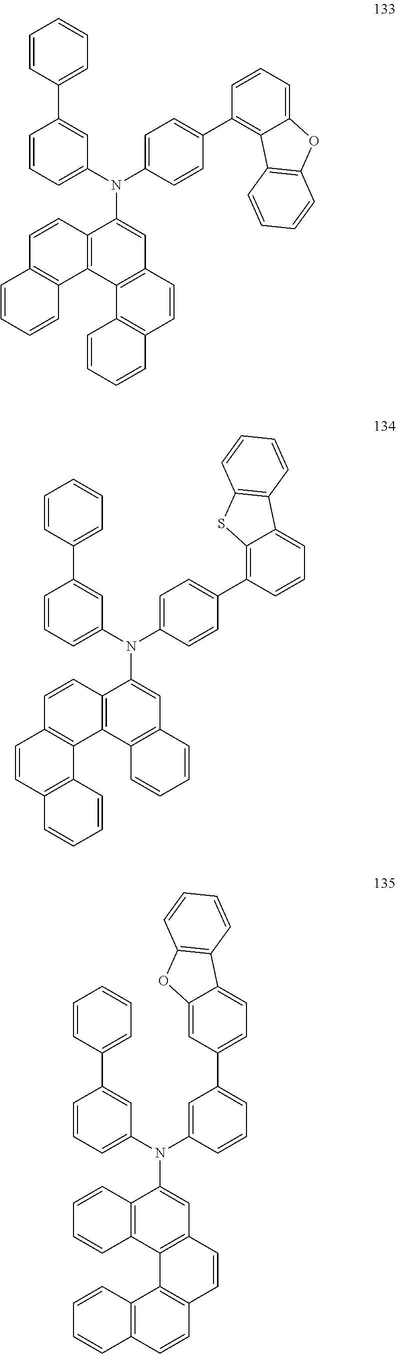 [SMILES: c1ccc(-c2cccc(N(c3ccc(-c4cccc5oc6ccccc6c45)cc3)c3cc4ccc5ccccc5c4c4c3ccc3ccccc34)c2)cc1 ; c1ccc(-c2cccc(N(c3ccc(-c4cccc5c4sc4ccccc45)cc3)c3cc4ccccc4c4c3ccc3ccc5ccccc5c34)c2)cc1 ; c1ccc(-c2cccc(N(c3cccc(-c4ccc5c(c4)oc4ccccc45)c3)c3cc4ccc5ccc6ccccc6c5c4c4ccccc34)c2)cc1]

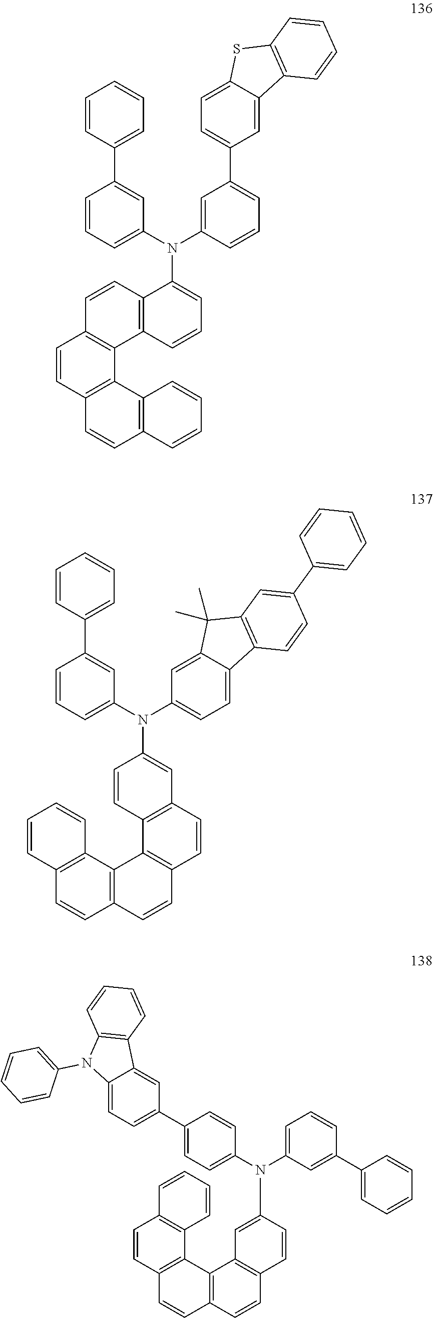 [SMILES: c1ccc(-c2cccc(N(c3cccc(-c4ccc5sc6ccccc6c5c4)c3)c3cccc4c3ccc3ccc5ccc6ccccc6c5c34)c2)cc1 ; CC1(C)c2cc(-c3ccccc3)ccc2-c2ccc(N(c3cccc(-c4ccccc4)c3)c3ccc4c(ccc5ccc6ccc7ccccc7c6c54)c3)cc21 ; c1ccc(-c2cccc(N(c3ccc(-c4ccc5c(c4)c4ccccc4n5-c4ccccc4)cc3)c3ccc4ccc5ccc6ccc7ccccc7c6c5c4c3)c2)cc1]

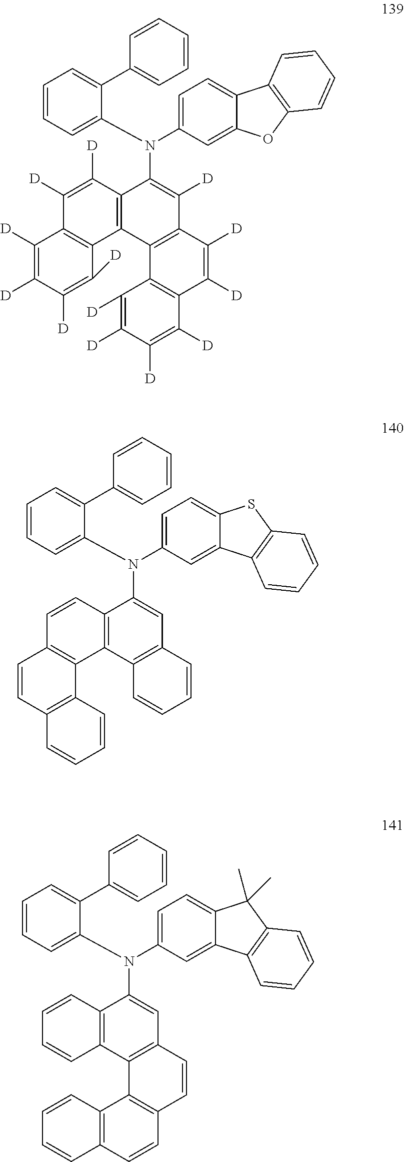 [SMILES: [2H]c1c([2H])c([2H])c2c(c1[2H])c([2H])c([2H])c1c([2H])c(N(c3ccc4c(c3)oc3ccccc34)c3ccccc3-c3ccccc3)c3c([2H])c([2H])c4c([2H])c([2H])c([2H])c([2H])c4c3c12 ; c1ccc(-c2ccccc2N(c2ccc3sc4ccccc4c3c2)c2cc3ccccc3c3c2ccc2ccc4ccccc4c23)cc1 ; CC1(C)c2ccccc2-c2cc(N(c3ccccc3-c3ccccc3)c3cc4ccc5ccc6ccccc6c5c4c4ccccc34)ccc21]

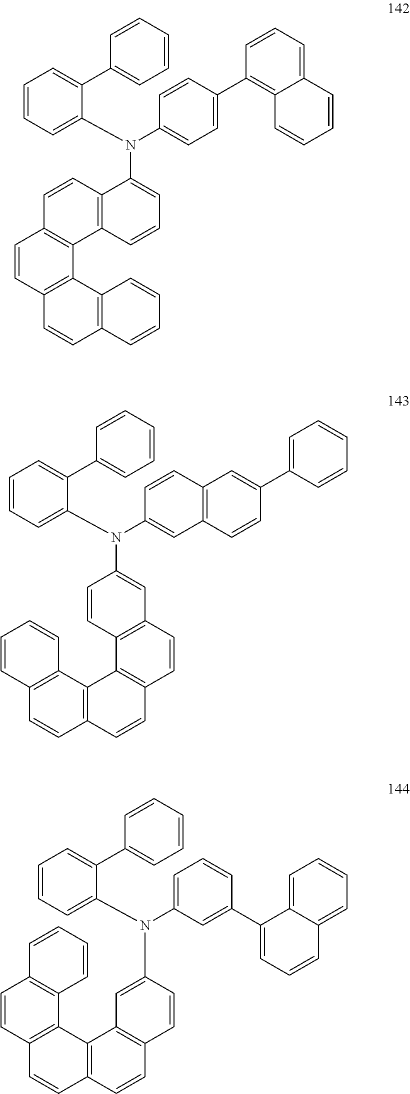 [SMILES: c1ccc(-c2ccccc2N(c2ccc(-c3cccc4ccccc34)cc2)c2cccc3c2ccc2ccc4ccc5ccccc5c4c23)cc1 ; c1ccc(-c2ccc3cc(N(c4ccc5c(ccc6ccc7ccc8ccccc8c7c65)c4)c4ccccc4-c4ccccc4)ccc3c2)cc1 ; c1ccc(-c2ccccc2N(c2cccc(-c3cccc4ccccc34)c2)c2ccc3ccc4ccc5ccc6ccccc6c5c4c3c2)cc1]

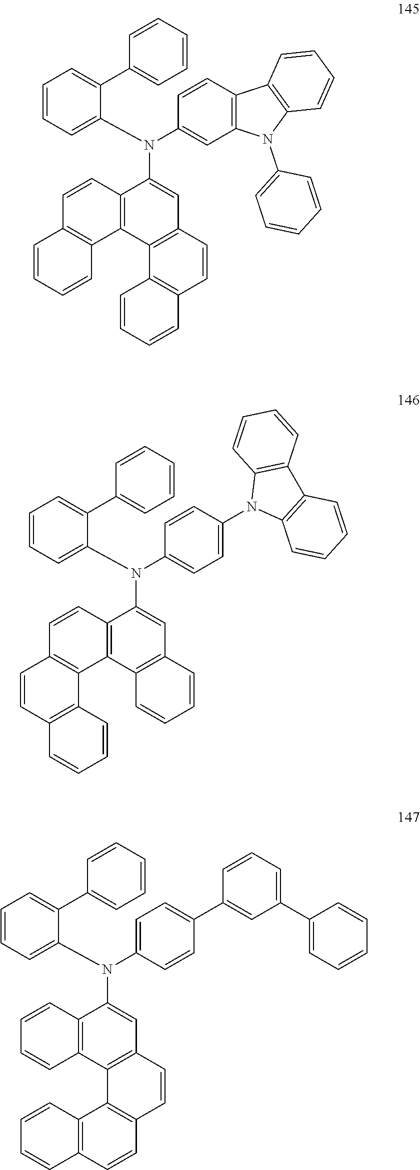 [SMILES: c1ccc(-c2ccccc2N(c2ccc3c4ccccc4n(-c4ccccc4)c3c2)c2cc3ccc4ccccc4c3c3c2ccc2ccccc23)cc1 ; c1ccc(-c2ccccc2N(c2ccc(-n3c4ccccc4c4ccccc43)cc2)c2cc3ccccc3c3c2ccc2ccc4ccccc4c23)cc1 ; c1ccc(-c2cccc(-c3ccc(N(c4ccccc4-c4ccccc4)c4cc5ccc6ccc7ccccc7c6c5c5ccccc45)cc3)c2)cc1]

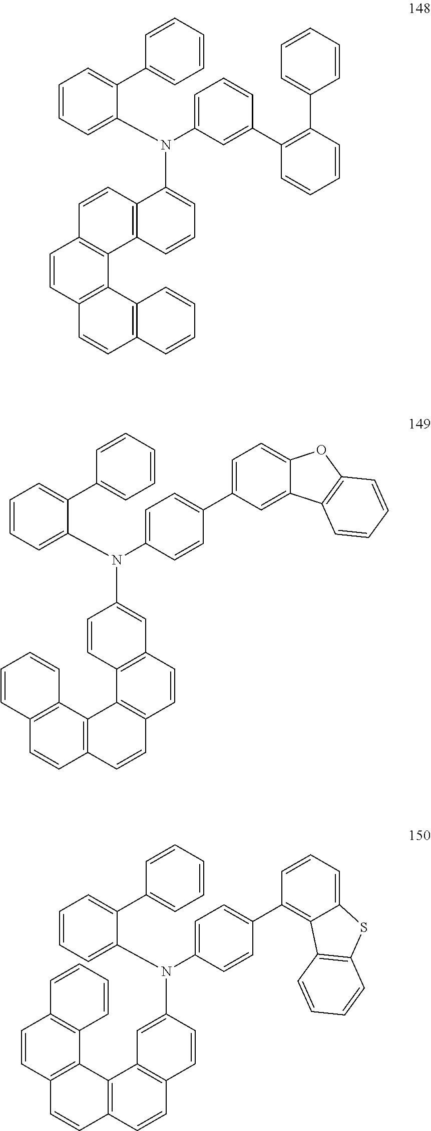 [SMILES: c1ccc(-c2ccccc2-c2cccc(N(c3ccccc3-c3ccccc3)c3cccc4c3ccc3ccc5ccc6ccccc6c5c34)c2)cc1 ; c1ccc(-c2ccccc2N(c2ccc(-c3ccc4oc5ccccc5c4c3)cc2)c2ccc3c(ccc4ccc5ccc6ccccc6c5c43)c2)cc1 ; c1ccc(-c2ccccc2N(c2ccc(-c3cccc4sc5ccccc5c34)cc2)c2ccc3ccc4ccc5ccc6ccccc6c5c4c3c2)cc1]

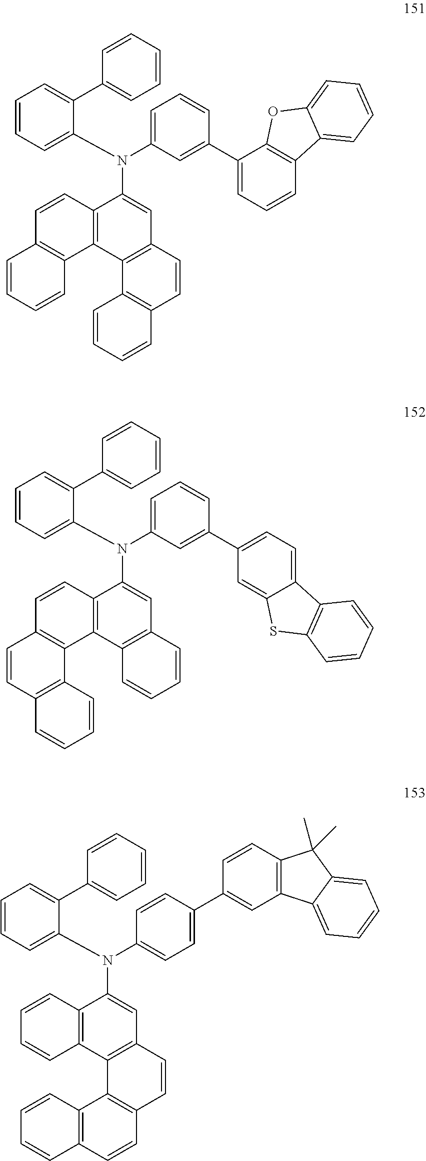 [SMILES: c1ccc(-c2ccccc2N(c2cccc(-c3cccc4c3oc3ccccc34)c2)c2cc3ccc4ccccc4c3c3c2ccc2ccccc23)cc1 ; c1ccc(-c2ccccc2N(c2cccc(-c3ccc4c(c3)sc3ccccc34)c2)c2cc3ccccc3c3c2ccc2ccc4ccccc4c23)cc1 ; CC1(C)c2ccccc2-c2cc(-c3ccc(N(c4ccccc4-c4ccccc4)c4cc5ccc6ccc7ccccc7c6c5c5ccccc45)cc3)ccc21]

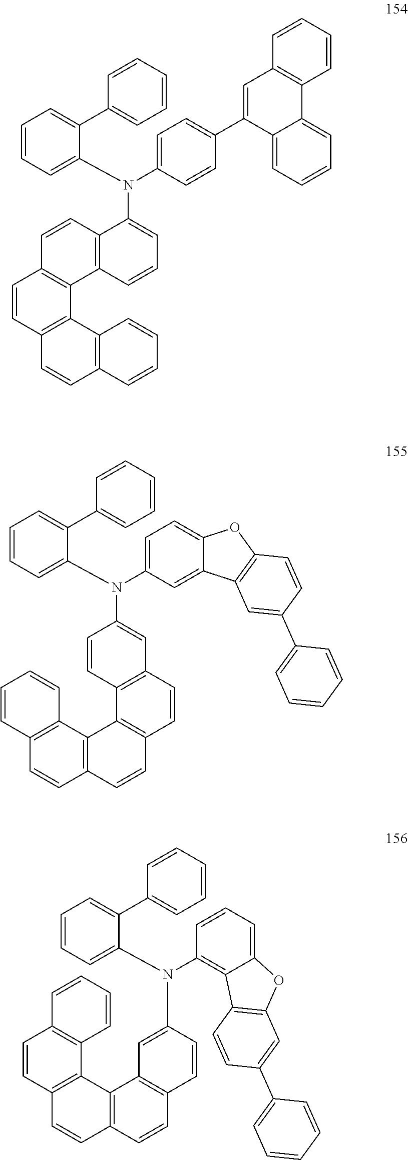 [SMILES: c1ccc(-c2ccccc2N(c2ccc(-c3cc4ccccc4c4ccccc34)cc2)c2cccc3c2ccc2ccc4ccc5ccccc5c4c23)cc1 ; c1ccc(-c2ccc3oc4ccc(N(c5ccc6c(ccc7ccc8ccc9ccccc9c8c76)c5)c5ccccc5-c5ccccc5)cc4c3c2)cc1 ; c1ccc(-c2ccc3c(c2)oc2cccc(N(c4ccc5ccc6ccc7ccc8ccccc8c7c6c5c4)c4ccccc4-c4ccccc4)c23)cc1]

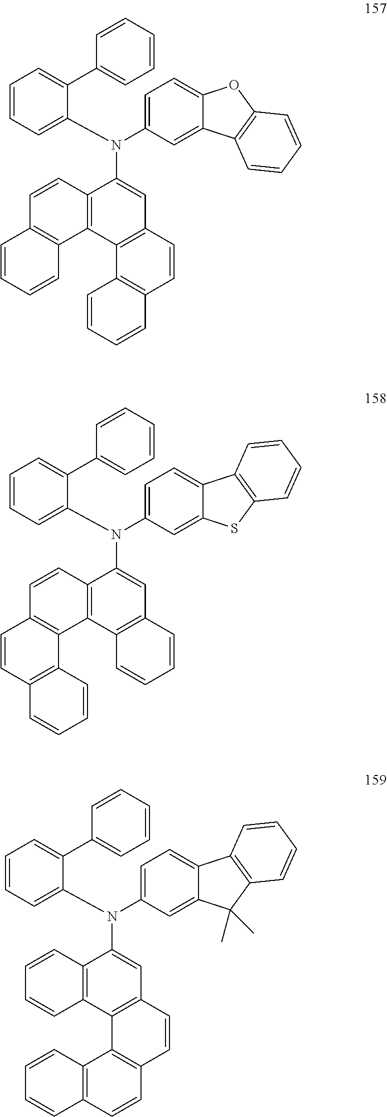 [SMILES: c1ccc(-c2ccccc2N(c2ccc3oc4ccccc4c3c2)c2cc3ccc4ccccc4c3c3c2ccc2ccccc23)cc1 ; c1ccc(-c2ccccc2N(c2ccc3c(c2)sc2ccccc23)c2cc3ccccc3c3c2ccc2ccc4ccccc4c23)cc1 ; CC1(C)c2ccccc2-c2ccc(N(c3ccccc3-c3ccccc3)c3cc4ccc5ccc6ccccc6c5c4c4ccccc34)cc21]

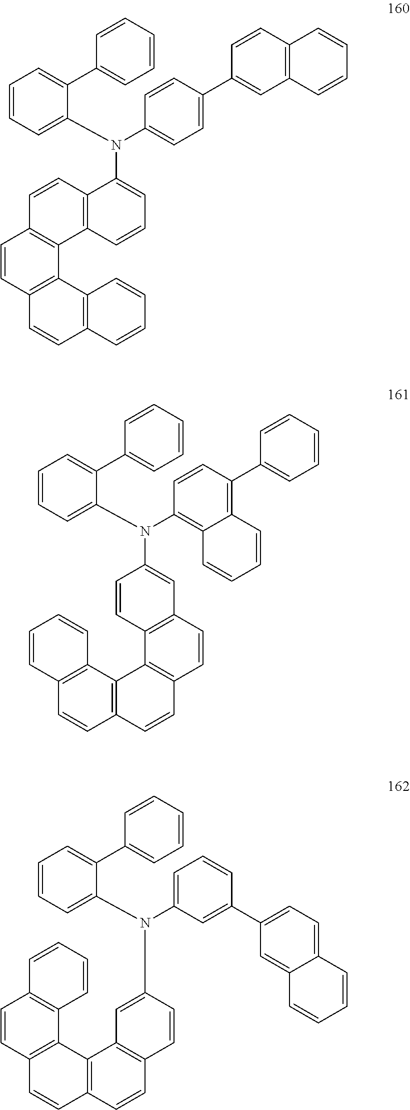 [SMILES: c1ccc(-c2ccccc2N(c2ccc(-c3ccc4ccccc4c3)cc2)c2cccc3c2ccc2ccc4ccc5ccccc5c4c23)cc1 ; c1ccc(-c2ccccc2N(c2ccc3c(ccc4ccc5ccc6ccccc6c5c43)c2)c2ccc(-c3ccccc3)c3ccccc23)cc1 ; c1ccc(-c2ccccc2N(c2cccc(-c3ccc4ccccc4c3)c2)c2ccc3ccc4ccc5ccc6ccccc6c5c4c3c2)cc1]

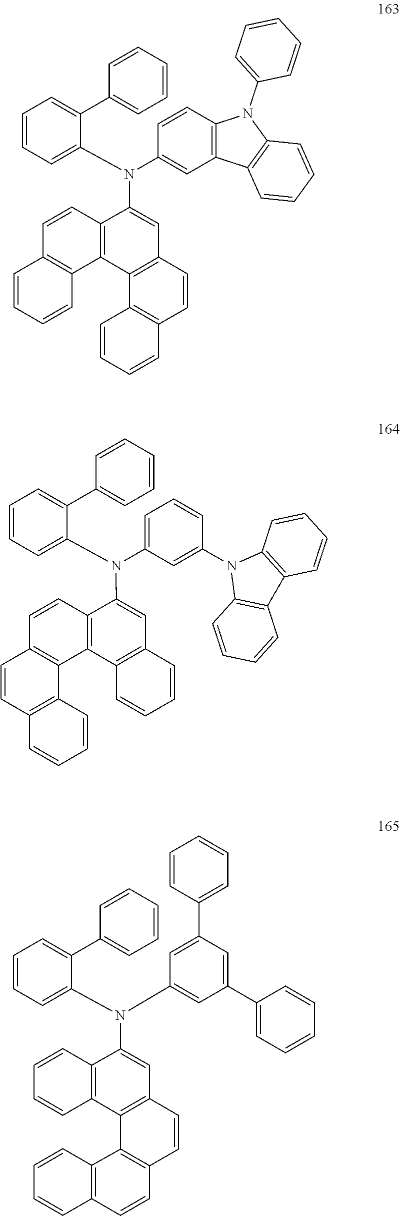 [SMILES: c1ccc(-c2ccccc2N(c2ccc3c(c2)c2ccccc2n3-c2ccccc2)c2cc3ccc4ccccc4c3c3c2ccc2ccccc23)cc1 ; c1ccc(-c2ccccc2N(c2cccc(-n3c4ccccc4c4ccccc43)c2)c2cc3ccccc3c3c2ccc2ccc4ccccc4c23)cc1 ; c1ccc(-c2cc(-c3ccccc3)cc(N(c3ccccc3-c3ccccc3)c3cc4ccc5ccc6ccccc6c5c4c4ccccc34)c2)cc1]

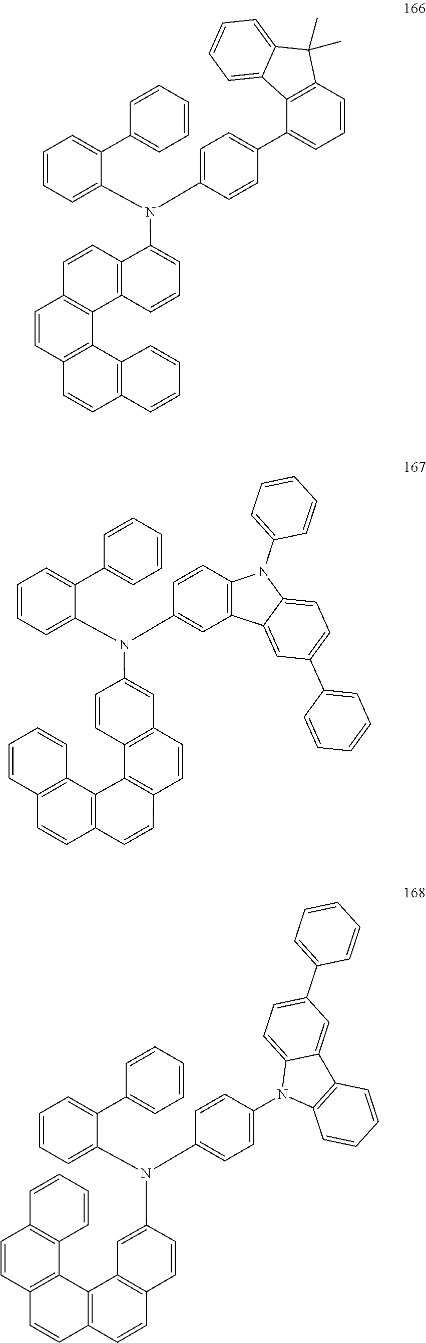 [SMILES: CC1(C)c2ccccc2-c2c(-c3ccc(N(c4ccccc4-c4ccccc4)c4cccc5c4ccc4ccc6ccc7ccccc7c6c45)cc3)cccc21 ; c1ccc(-c2ccc3c(c2)c2cc(N(c4ccc5c(ccc6ccc7ccc8ccccc8c7c65)c4)c4ccccc4-c4ccccc4)ccc2n3-c2ccccc2)cc1 ; c1ccc(-c2ccc3c(c2)c2ccccc2n3-c2ccc(N(c3ccc4ccc5ccc6ccc7ccccc7c6c5c4c3)c3ccccc3-c3ccccc3)cc2)cc1]

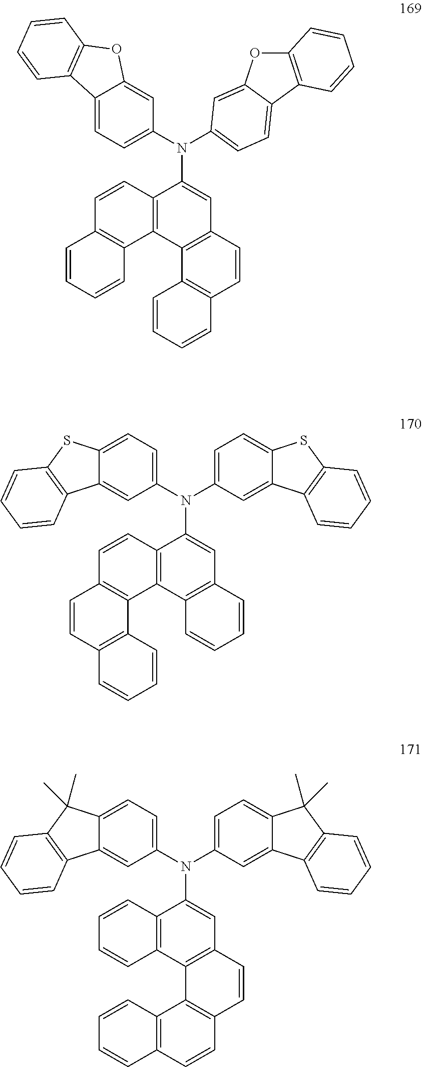 [SMILES: c1ccc2c(c1)ccc1cc(N(c3ccc4c(c3)oc3ccccc34)c3ccc4c(c3)oc3ccccc34)c3ccc4ccccc4c3c12 ; c1ccc2c(c1)ccc1ccc3c(N(c4ccc5sc6ccccc6c5c4)c4ccc5sc6ccccc6c5c4)cc4ccccc4c3c12 ; CC1(C)c2ccccc2-c2cc(N(c3ccc4c(c3)-c3ccccc3C4(C)C)c3cc4ccc5ccc6ccccc6c5c4c4ccccc34)ccc21]

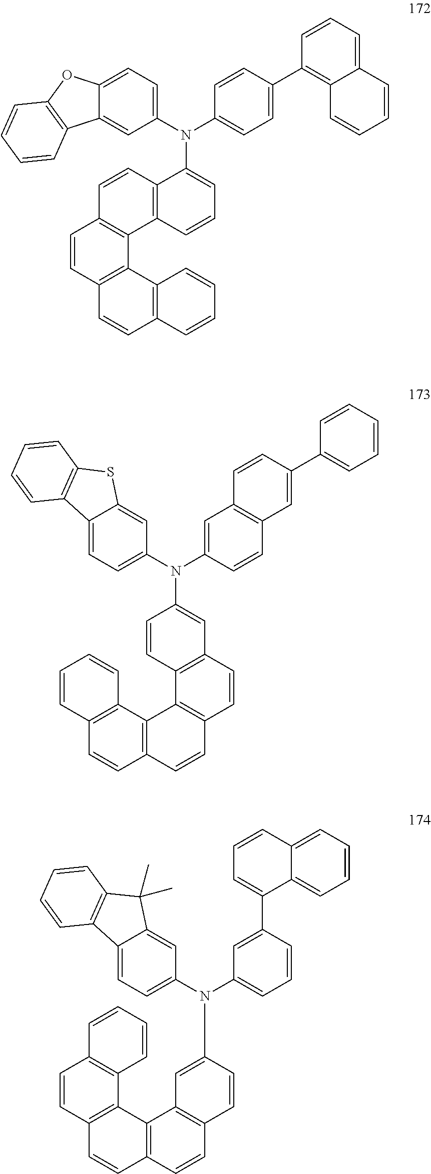 [SMILES: c1ccc2c(-c3ccc(N(c4ccc5oc6ccccc6c5c4)c4cccc5c4ccc4ccc6ccc7ccccc7c6c45)cc3)cccc2c1 ; c1ccc(-c2ccc3cc(N(c4ccc5c(ccc6ccc7ccc8ccccc8c7c65)c4)c4ccc5c(c4)sc4ccccc45)ccc3c2)cc1 ; CC1(C)c2ccccc2-c2ccc(N(c3cccc(-c4cccc5ccccc45)c3)c3ccc4ccc5ccc6ccc7ccccc7c6c5c4c3)cc21]

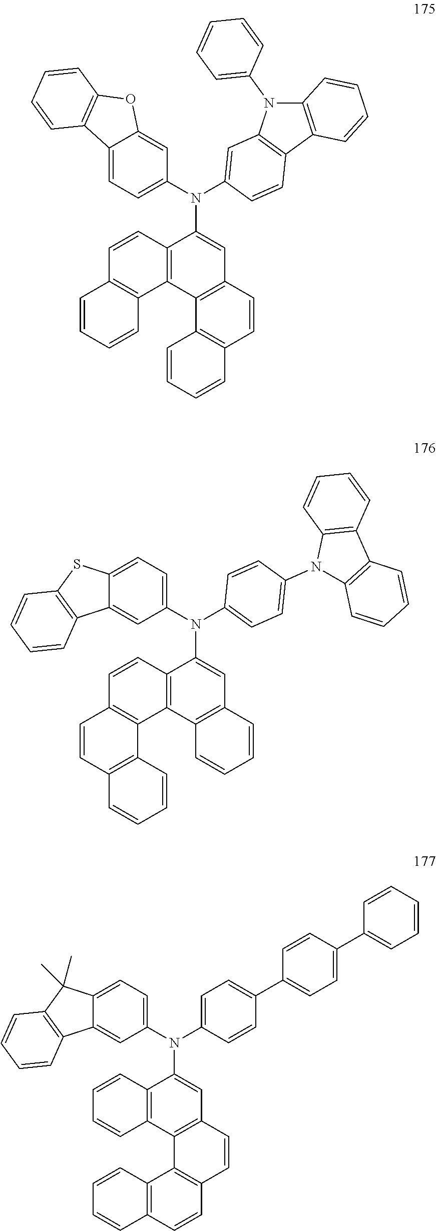 [SMILES: c1ccc(-n2c3ccccc3c3ccc(N(c4ccc5c(c4)oc4ccccc45)c4cc5ccc6ccccc6c5c5c4ccc4ccccc45)cc32)cc1 ; c1ccc2c(c1)ccc1ccc3c(N(c4ccc(-n5c6ccccc6c6ccccc65)cc4)c4ccc5sc6ccccc6c5c4)cc4ccccc4c3c12 ; CC1(C)c2ccccc2-c2cc(N(c3ccc(-c4ccc(-c5ccccc5)cc4)cc3)c3cc4ccc5ccc6ccccc6c5c4c4ccccc34)ccc21]

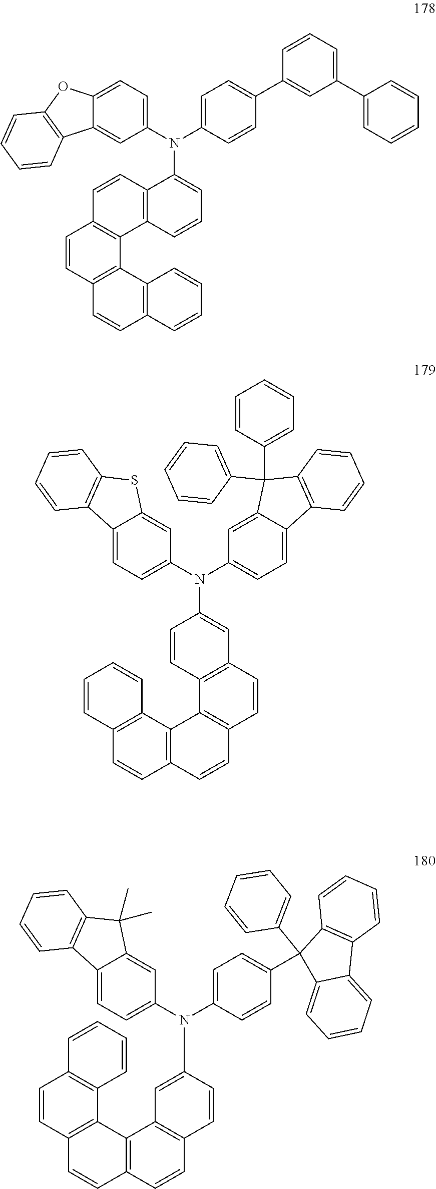 [SMILES: c1ccc(-c2cccc(-c3ccc(N(c4ccc5oc6ccccc6c5c4)c4cccc5c4ccc4ccc6ccc7ccccc7c6c45)cc3)c2)cc1 ; c1ccc(C2(c3ccccc3)c3ccccc3-c3ccc(N(c4ccc5c(ccc6ccc7ccc8ccccc8c7c65)c4)c4ccc5c(c4)sc4ccccc45)cc32)cc1 ; CC1(C)c2ccccc2-c2ccc(N(c3ccc(C4(c5ccccc5)c5ccccc5-c5ccccc54)cc3)c3ccc4ccc5ccc6ccc7ccccc7c6c5c4c3)cc21]

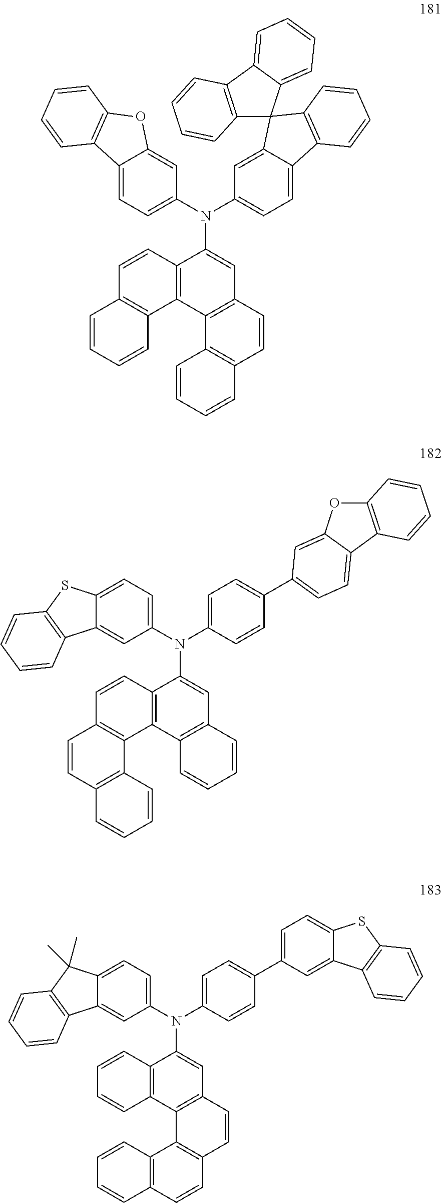 [SMILES: c1ccc2c(c1)-c1ccccc1C21c2ccccc2-c2ccc(N(c3ccc4c(c3)oc3ccccc34)c3cc4ccc5ccccc5c4c4c3ccc3ccccc34)cc21 ; c1ccc2c(c1)ccc1ccc3c(N(c4ccc(-c5ccc6c(c5)oc5ccccc56)cc4)c4ccc5sc6ccccc6c5c4)cc4ccccc4c3c12 ; CC1(C)c2ccccc2-c2cc(N(c3ccc(-c4ccc5sc6ccccc6c5c4)cc3)c3cc4ccc5ccc6ccccc6c5c4c4ccccc34)ccc21]

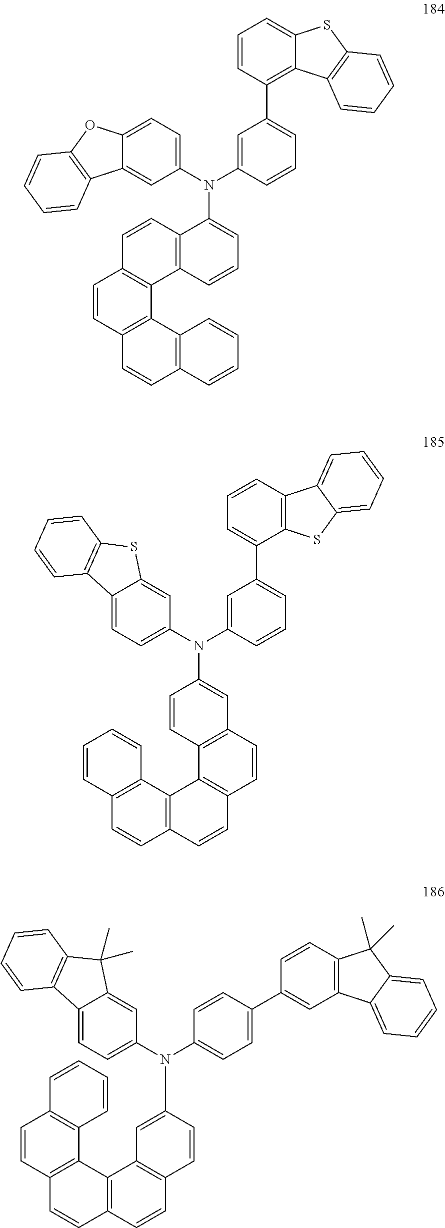 [SMILES: c1cc(-c2cccc3sc4ccccc4c23)cc(N(c2ccc3oc4ccccc4c3c2)c2cccc3c2ccc2ccc4ccc5ccccc5c4c23)c1 ; c1cc(-c2cccc3c2sc2ccccc23)cc(N(c2ccc3c(ccc4ccc5ccc6ccccc6c5c43)c2)c2ccc3c(c2)sc2ccccc23)c1 ; CC1(C)c2ccccc2-c2cc(-c3ccc(N(c4ccc5c(c4)C(C)(C)c4ccccc4-5)c4ccc5ccc6ccc7ccc8ccccc8c7c6c5c4)cc3)ccc21]

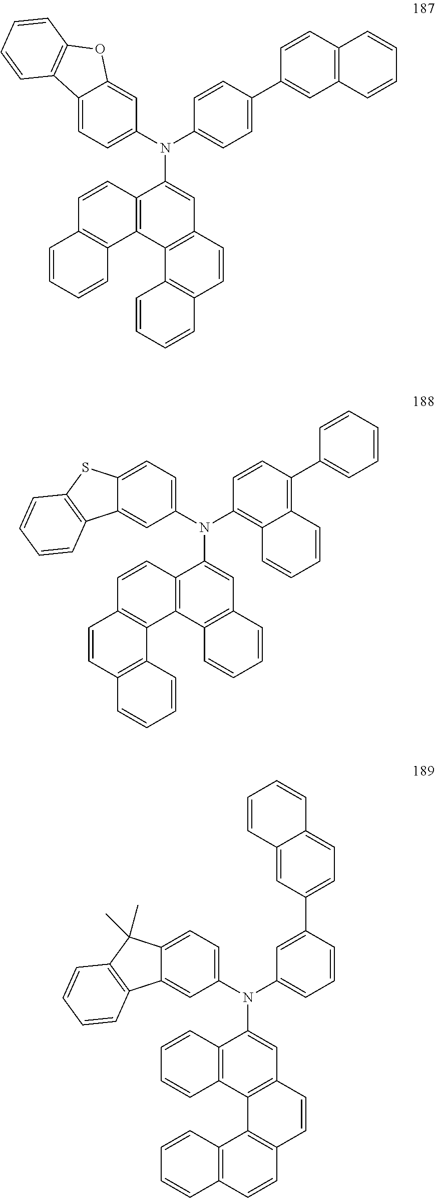 [SMILES: c1ccc2cc(-c3ccc(N(c4ccc5c(c4)oc4ccccc45)c4cc5ccc6ccccc6c5c5c4ccc4ccccc45)cc3)ccc2c1 ; c1ccc(-c2ccc(N(c3ccc4sc5ccccc5c4c3)c3cc4ccccc4c4c3ccc3ccc5ccccc5c34)c3ccccc23)cc1 ; CC1(C)c2ccccc2-c2cc(N(c3cccc(-c4ccc5ccccc5c4)c3)c3cc4ccc5ccc6ccccc6c5c4c4ccccc34)ccc21]

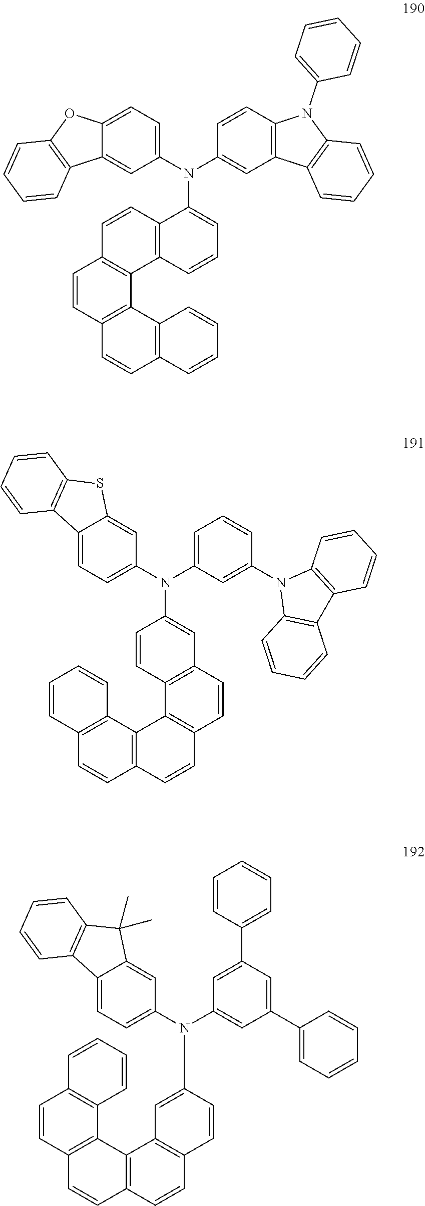 [SMILES: c1ccc(-n2c3ccccc3c3cc(N(c4ccc5oc6ccccc6c5c4)c4cccc5c4ccc4ccc6ccc7ccccc7c6c45)ccc32)cc1 ; c1cc(N(c2ccc3c(ccc4ccc5ccc6ccccc6c5c43)c2)c2ccc3c(c2)sc2ccccc23)cc(-n2c3ccccc3c3ccccc32)c1 ; CC1(C)c2ccccc2-c2ccc(N(c3cc(-c4ccccc4)cc(-c4ccccc4)c3)c3ccc4ccc5ccc6ccc7ccccc7c6c5c4c3)cc21]

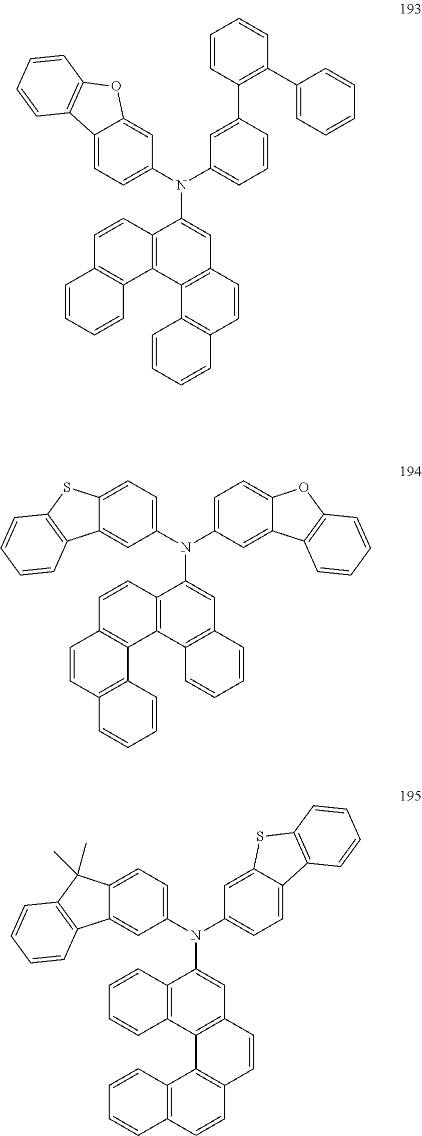 [SMILES: c1ccc(-c2ccccc2-c2cccc(N(c3ccc4c(c3)oc3ccccc34)c3cc4ccc5ccccc5c4c4c3ccc3ccccc34)c2)cc1 ; c1ccc2c(c1)ccc1ccc3c(N(c4ccc5oc6ccccc6c5c4)c4ccc5sc6ccccc6c5c4)cc4ccccc4c3c12 ; CC1(C)c2ccccc2-c2cc(N(c3ccc4c(c3)sc3ccccc34)c3cc4ccc5ccc6ccccc6c5c4c4ccccc34)ccc21]

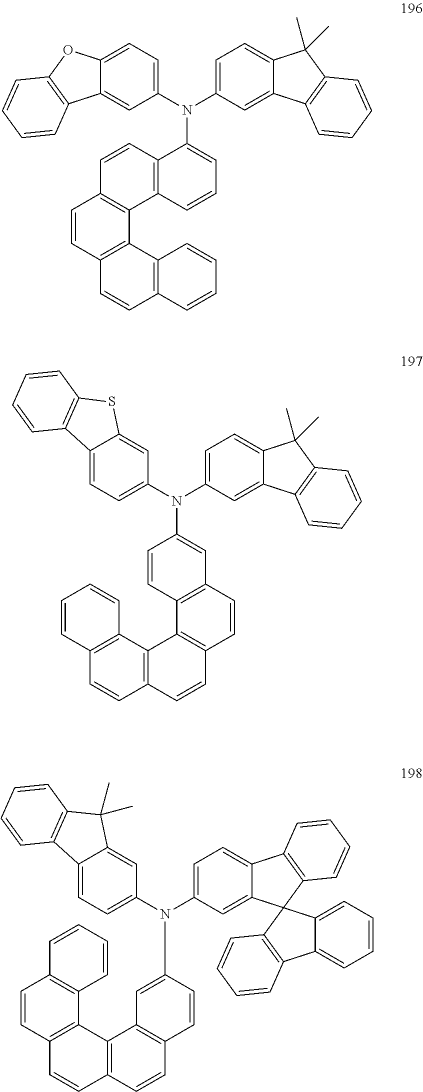 [SMILES: CC1(C)c2ccccc2-c2cc(N(c3ccc4oc5ccccc5c4c3)c3cccc4c3ccc3ccc5ccc6ccccc6c5c34)ccc21 ; CC1(C)c2ccccc2-c2cc(N(c3ccc4c(ccc5ccc6ccc7ccccc7c6c54)c3)c3ccc4c(c3)sc3ccccc34)ccc21 ; CC1(C)c2ccccc2-c2ccc(N(c3ccc4c(c3)C3(c5ccccc5-c5ccccc53)c3ccccc3-4)c3ccc4ccc5ccc6ccc7ccccc7c6c5c4c3)cc21]

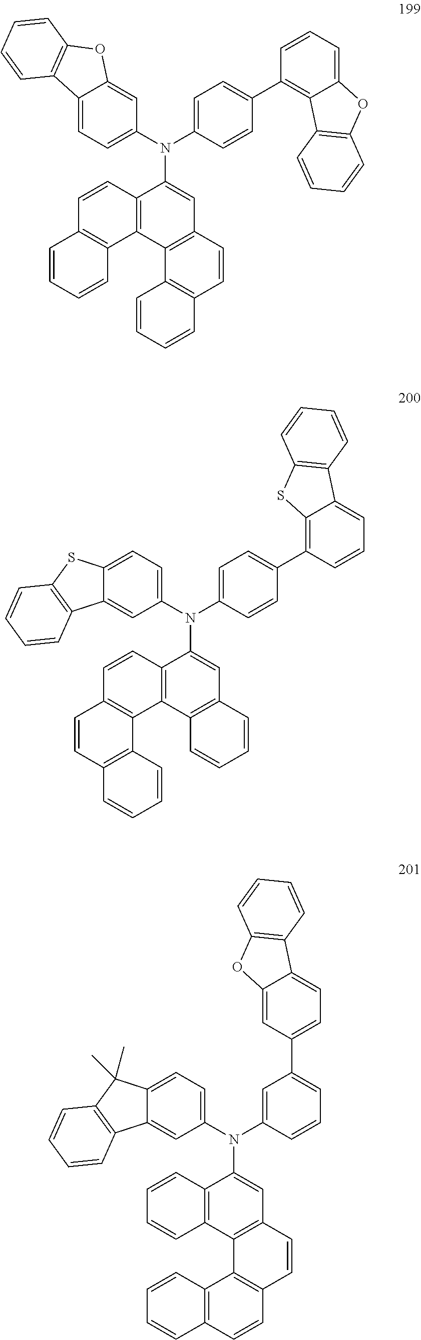 [SMILES: c1ccc2c(c1)ccc1cc(N(c3ccc(-c4cccc5oc6ccccc6c45)cc3)c3ccc4c(c3)oc3ccccc34)c3ccc4ccccc4c3c12 ; c1ccc2c(c1)ccc1ccc3c(N(c4ccc(-c5cccc6c5sc5ccccc56)cc4)c4ccc5sc6ccccc6c5c4)cc4ccccc4c3c12 ; CC1(C)c2ccccc2-c2cc(N(c3cccc(-c4ccc5c(c4)oc4ccccc45)c3)c3cc4ccc5ccc6ccccc6c5c4c4ccccc34)ccc21]

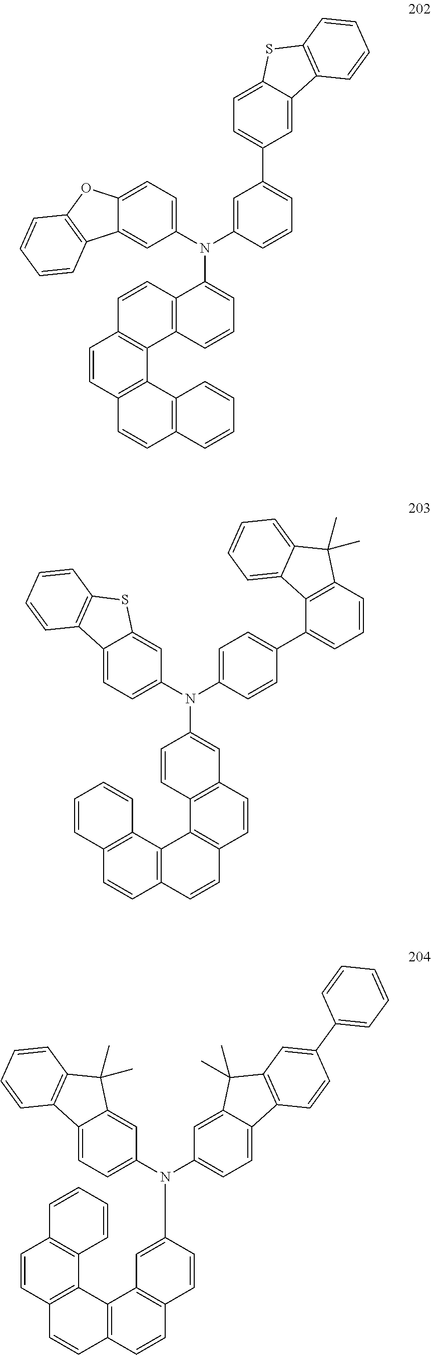 [SMILES: c1cc(-c2ccc3sc4ccccc4c3c2)cc(N(c2ccc3oc4ccccc4c3c2)c2cccc3c2ccc2ccc4ccc5ccccc5c4c23)c1 ; CC1(C)c2ccccc2-c2c(-c3ccc(N(c4ccc5c(ccc6ccc7ccc8ccccc8c7c65)c4)c4ccc5c(c4)sc4ccccc45)cc3)cccc21 ; CC1(C)c2ccccc2-c2ccc(N(c3ccc4c(c3)C(C)(C)c3cc(-c5ccccc5)ccc3-4)c3ccc4ccc5ccc6ccc7ccccc7c6c5c4c3)cc21]

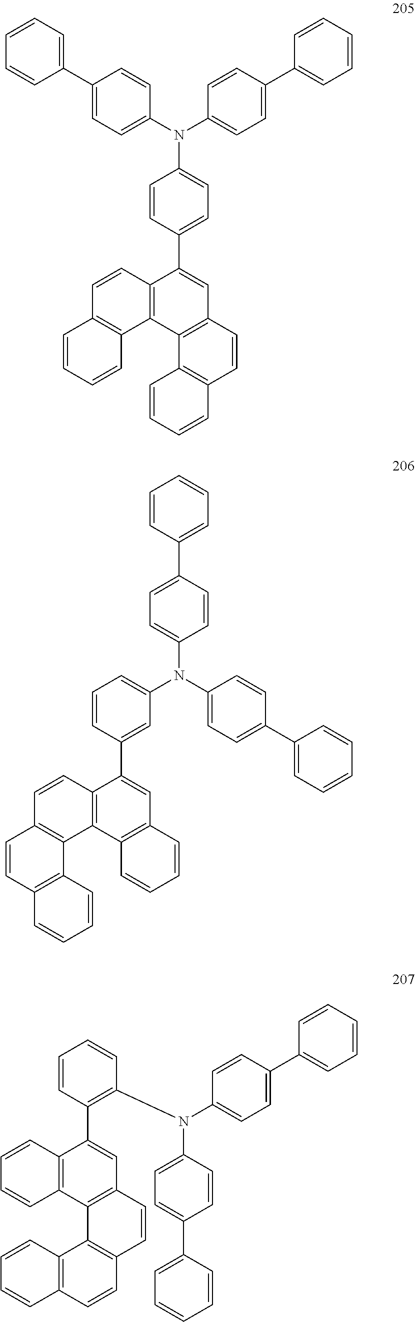

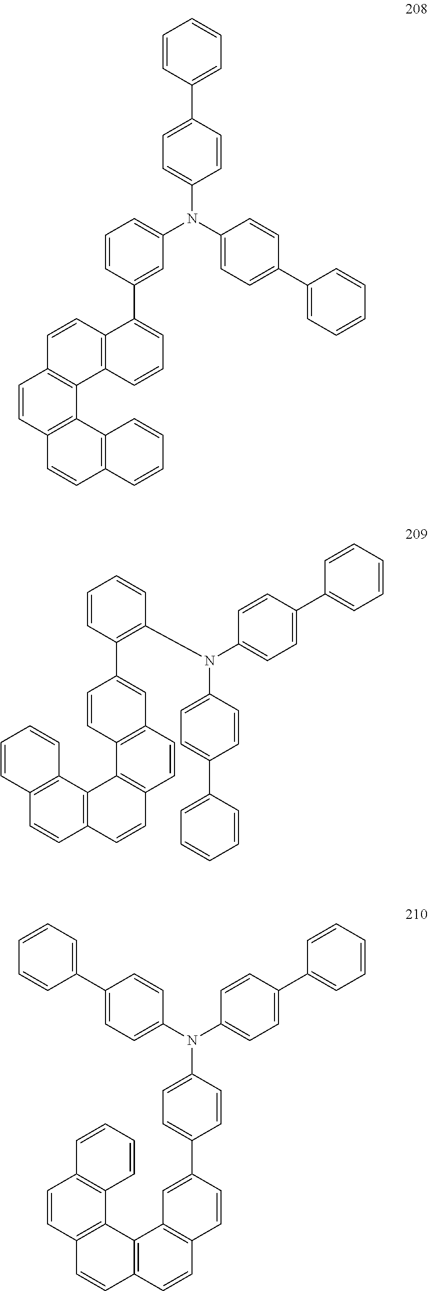 [SMILES: c1ccc(-c2ccc(N(c3ccc(-c4ccccc4)cc3)c3cccc(-c4cccc5c4ccc4ccc6ccc7ccccc7c6c45)c3)cc2)cc1 ; c1ccc(-c2ccc(N(c3ccc(-c4ccccc4)cc3)c3ccccc3-c3ccc4c(ccc5ccc6ccc7ccccc7c6c54)c3)cc2)cc1 ; c1ccc(-c2ccc(N(c3ccc(-c4ccccc4)cc3)c3ccc(-c4ccc5ccc6ccc7ccc8ccccc8c7c6c5c4)cc3)cc2)cc1]

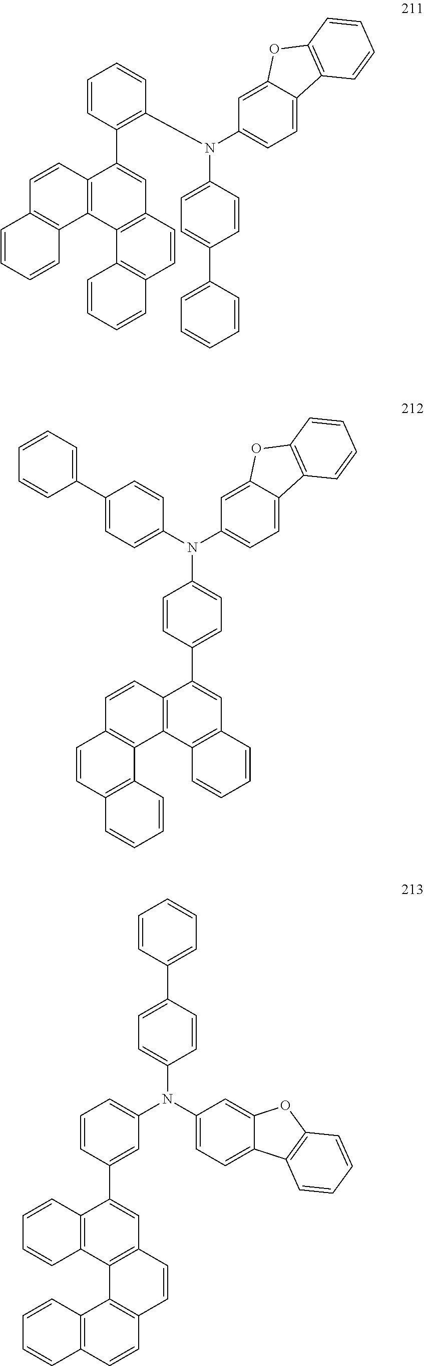 [SMILES: c1ccc(-c2ccc(N(c3ccc4c(c3)oc3ccccc34)c3ccccc3-c3cc4ccc5ccccc5c4c4c3ccc3ccccc34)cc2)cc1 ; c1ccc(-c2ccc(N(c3ccc(-c4cc5ccccc5c5c4ccc4ccc6ccccc6c45)cc3)c3ccc4c(c3)oc3ccccc34)cc2)cc1 ; c1ccc(-c2ccc(N(c3cccc(-c4cc5ccc6ccc7ccccc7c6c5c5ccccc45)c3)c3ccc4c(c3)oc3ccccc34)cc2)cc1]

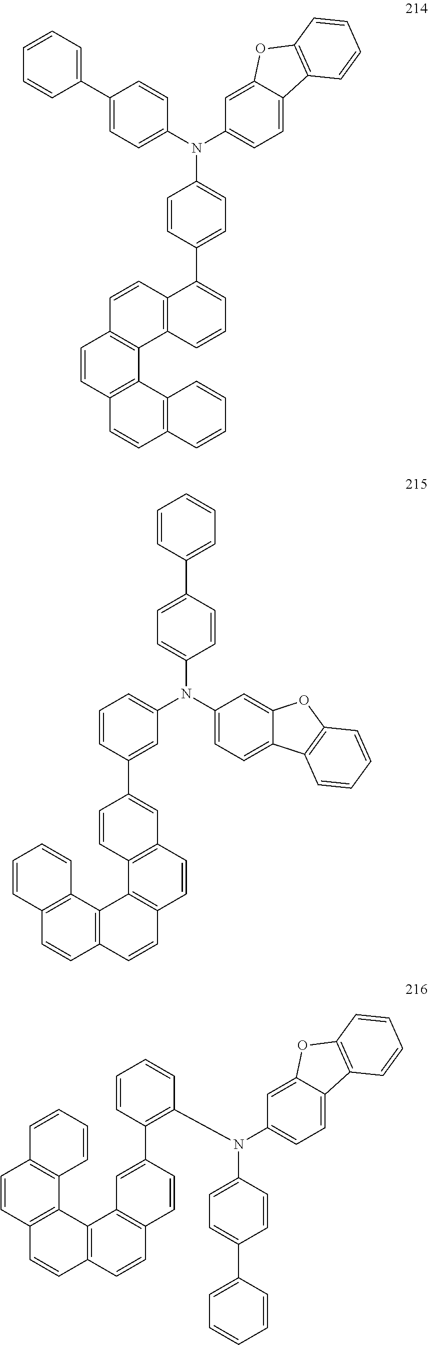 [SMILES: c1ccc(-c2ccc(N(c3ccc(-c4cccc5c4ccc4ccc6ccc7ccccc7c6c45)cc3)c3ccc4c(c3)oc3ccccc34)cc2)cc1 ; c1ccc(-c2ccc(N(c3cccc(-c4ccc5c(ccc6ccc7ccc8ccccc8c7c65)c4)c3)c3ccc4c(c3)oc3ccccc34)cc2)cc1 ; c1ccc(-c2ccc(N(c3ccc4c(c3)oc3ccccc34)c3ccccc3-c3ccc4ccc5ccc6ccc7ccccc7c6c5c4c3)cc2)cc1]

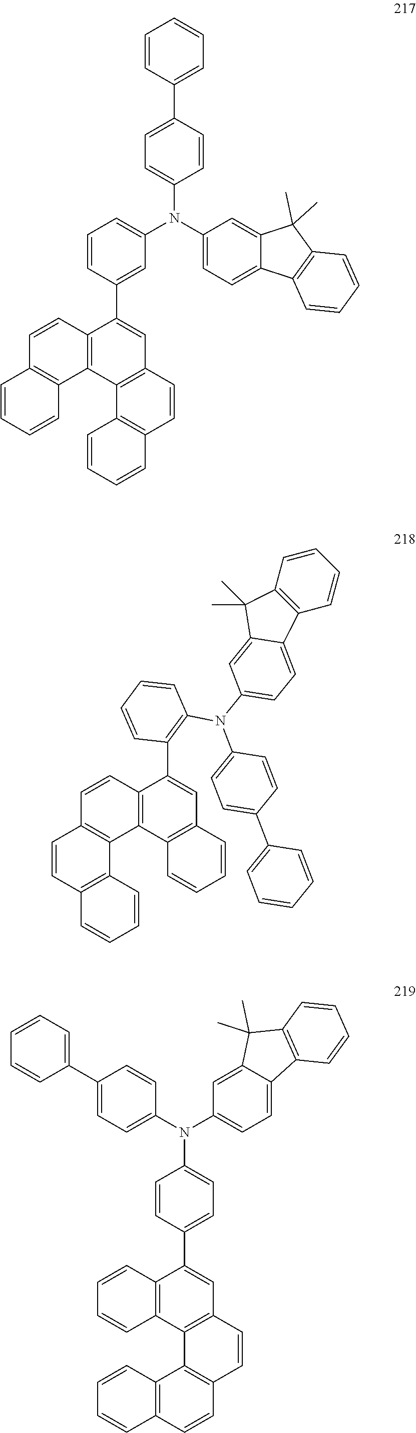 [SMILES: CC1(C)c2ccccc2-c2ccc(N(c3ccc(-c4ccccc4)cc3)c3cccc(-c4cc5ccc6ccccc6c5c5c4ccc4ccccc45)c3)cc21 ; CC1(C)c2ccccc2-c2ccc(N(c3ccc(-c4ccccc4)cc3)c3ccccc3-c3cc4ccccc4c4c3ccc3ccc5ccccc5c34)cc21 ; CC1(C)c2ccccc2-c2ccc(N(c3ccc(-c4ccccc4)cc3)c3ccc(-c4cc5ccc6ccc7ccccc7c6c5c5ccccc45)cc3)cc21]

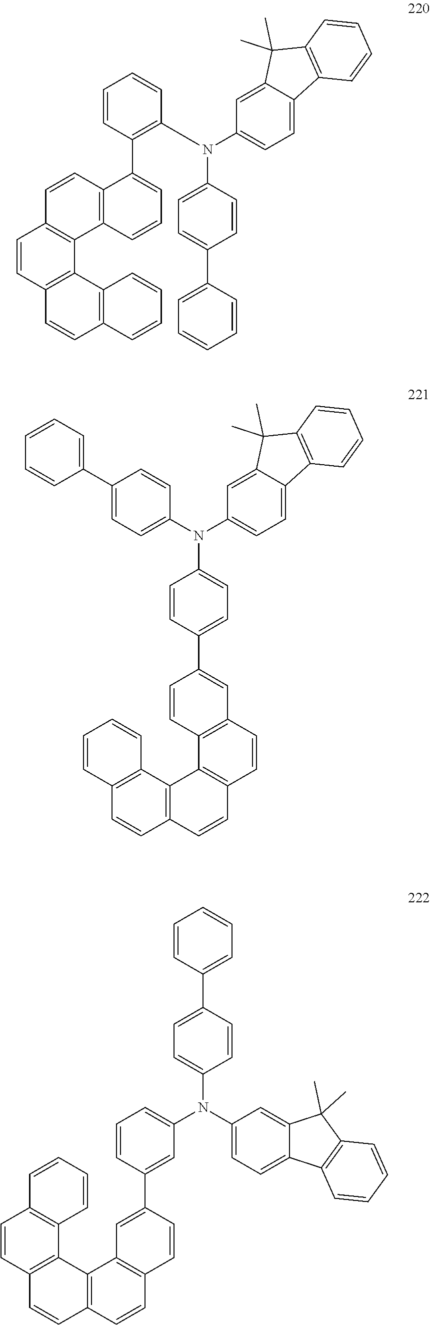 [SMILES: CC1(C)c2ccccc2-c2ccc(N(c3ccc(-c4ccccc4)cc3)c3ccccc3-c3cccc4c3ccc3ccc5ccc6ccccc6c5c34)cc21 ; CC1(C)c2ccccc2-c2ccc(N(c3ccc(-c4ccccc4)cc3)c3ccc(-c4ccc5c(ccc6ccc7ccc8ccccc8c7c65)c4)cc3)cc21 ; CC1(C)c2ccccc2-c2ccc(N(c3ccc(-c4ccccc4)cc3)c3cccc(-c4ccc5ccc6ccc7ccc8ccccc8c7c6c5c4)c3)cc21]

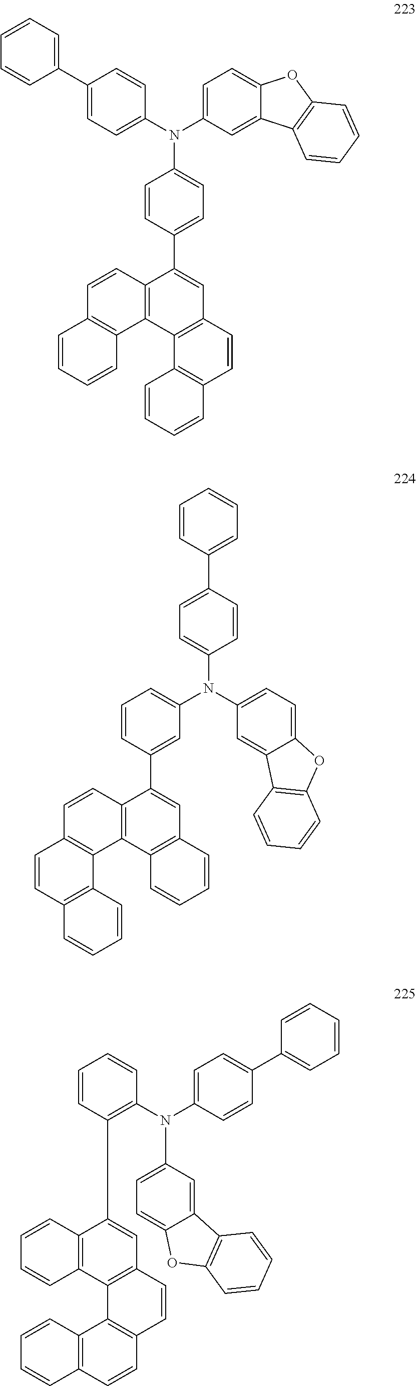 [SMILES: c1ccc(-c2ccc(N(c3ccc(-c4cc5ccc6ccccc6c5c5c4ccc4ccccc45)cc3)c3ccc4oc5ccccc5c4c3)cc2)cc1 ; c1ccc(-c2ccc(N(c3cccc(-c4cc5ccccc5c5c4ccc4ccc6ccccc6c45)c3)c3ccc4oc5ccccc5c4c3)cc2)cc1 ; c1ccc(-c2ccc(N(c3ccc4oc5ccccc5c4c3)c3ccccc3-c3cc4ccc5ccc6ccccc6c5c4c4ccccc34)cc2)cc1]

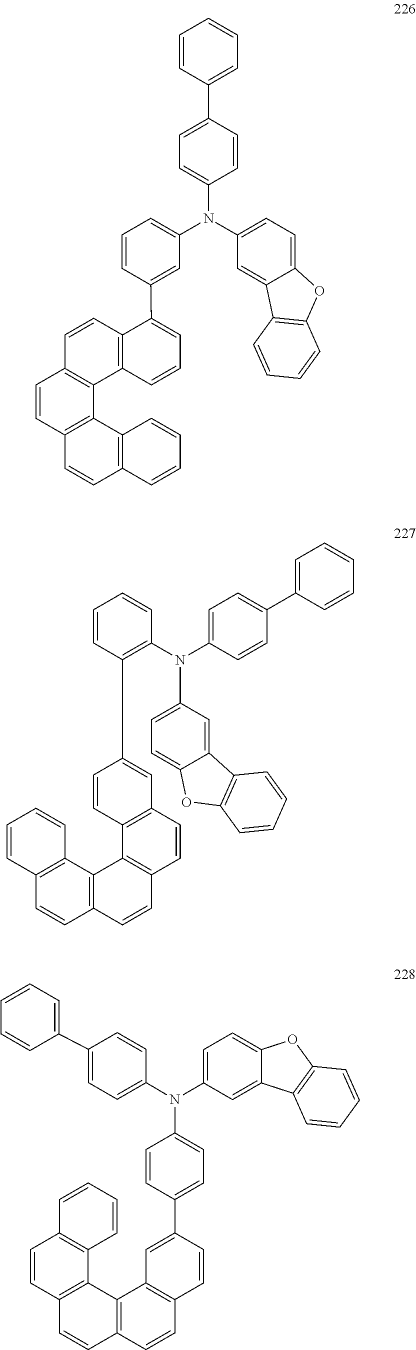 [SMILES: c1ccc(-c2ccc(N(c3cccc(-c4cccc5c4ccc4ccc6ccc7ccccc7c6c45)c3)c3ccc4oc5ccccc5c4c3)cc2)cc1 ; c1ccc(-c2ccc(N(c3ccc4oc5ccccc5c4c3)c3ccccc3-c3ccc4c(ccc5ccc6ccc7ccccc7c6c54)c3)cc2)cc1 ; c1ccc(-c2ccc(N(c3ccc(-c4ccc5ccc6ccc7ccc8ccccc8c7c6c5c4)cc3)c3ccc4oc5ccccc5c4c3)cc2)cc1]

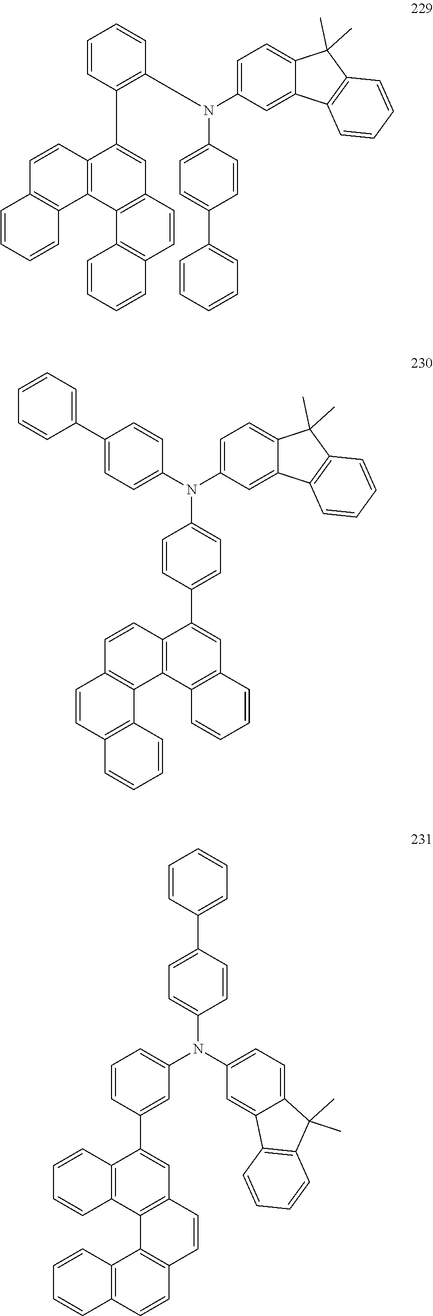 [SMILES: CC1(C)c2ccccc2-c2cc(N(c3ccc(-c4ccccc4)cc3)c3ccccc3-c3cc4ccc5ccccc5c4c4c3ccc3ccccc34)ccc21 ; CC1(C)c2ccccc2-c2cc(N(c3ccc(-c4ccccc4)cc3)c3ccc(-c4cc5ccccc5c5c4ccc4ccc6ccccc6c45)cc3)ccc21 ; CC1(C)c2ccccc2-c2cc(N(c3ccc(-c4ccccc4)cc3)c3cccc(-c4cc5ccc6ccc7ccccc7c6c5c5ccccc45)c3)ccc21]

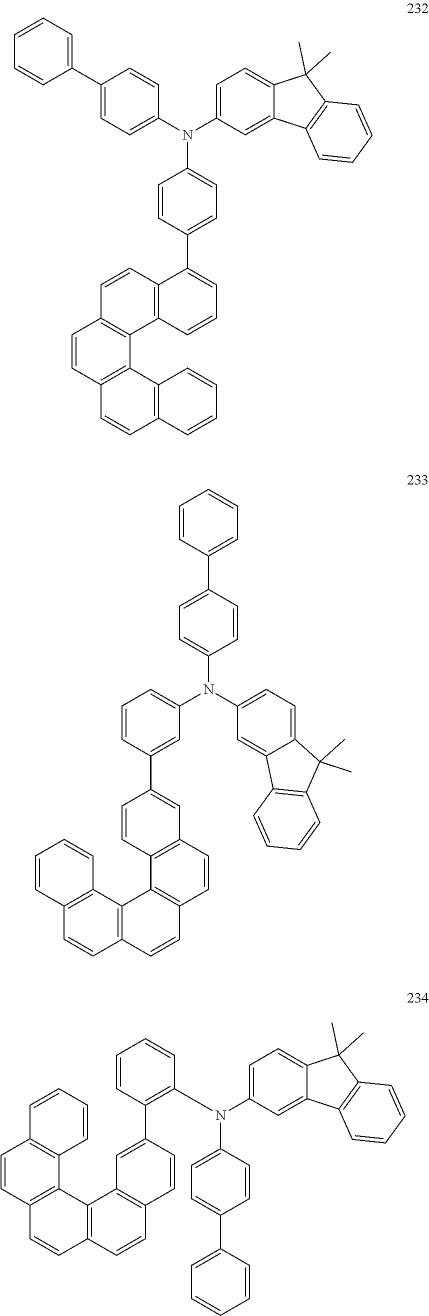 [SMILES: CC1(C)c2ccccc2-c2cc(N(c3ccc(-c4ccccc4)cc3)c3ccc(-c4cccc5c4ccc4ccc6ccc7ccccc7c6c45)cc3)ccc21 ; CC1(C)c2ccccc2-c2cc(N(c3ccc(-c4ccccc4)cc3)c3cccc(-c4ccc5c(ccc6ccc7ccc8ccccc8c7c65)c4)c3)ccc21 ; CC1(C)c2ccccc2-c2cc(N(c3ccc(-c4ccccc4)cc3)c3ccccc3-c3ccc4ccc5ccc6ccc7ccccc7c6c5c4c3)ccc21]

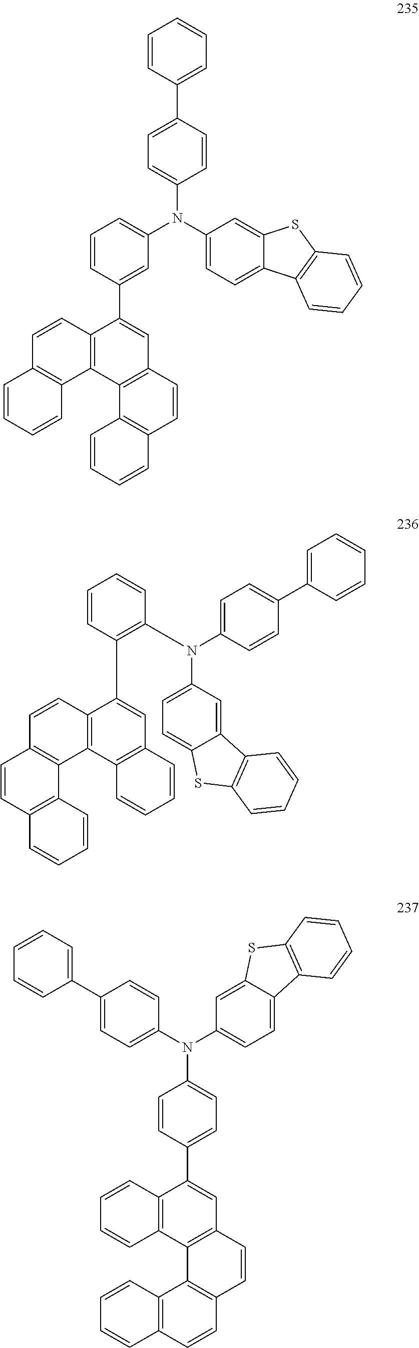 [SMILES: c1ccc(-c2ccc(N(c3cccc(-c4cc5ccc6ccccc6c5c5c4ccc4ccccc45)c3)c3ccc4c(c3)sc3ccccc34)cc2)cc1 ; c1ccc(-c2ccc(N(c3ccc4sc5ccccc5c4c3)c3ccccc3-c3cc4ccccc4c4c3ccc3ccc5ccccc5c34)cc2)cc1 ; c1ccc(-c2ccc(N(c3ccc(-c4cc5ccc6ccc7ccccc7c6c5c5ccccc45)cc3)c3ccc4c(c3)sc3ccccc34)cc2)cc1]

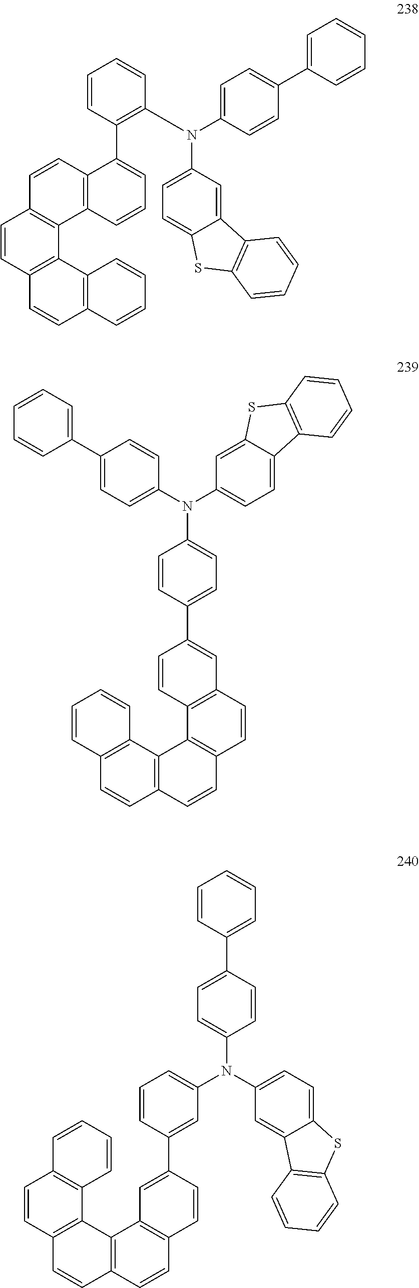 [SMILES: c1ccc(-c2ccc(N(c3ccc4sc5ccccc5c4c3)c3ccccc3-c3cccc4c3ccc3ccc5ccc6ccccc6c5c34)cc2)cc1 ; c1ccc(-c2ccc(N(c3ccc(-c4ccc5c(ccc6ccc7ccc8ccccc8c7c65)c4)cc3)c3ccc4c(c3)sc3ccccc34)cc2)cc1 ; c1ccc(-c2ccc(N(c3cccc(-c4ccc5ccc6ccc7ccc8ccccc8c7c6c5c4)c3)c3ccc4sc5ccccc5c4c3)cc2)cc1]

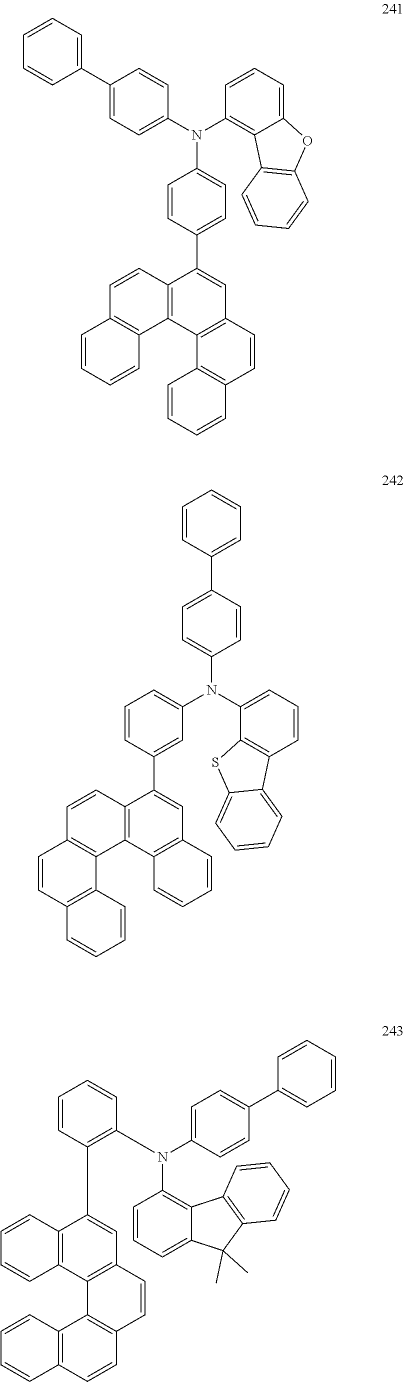 [SMILES: c1ccc(-c2ccc(N(c3ccc(-c4cc5ccc6ccccc6c5c5c4ccc4ccccc45)cc3)c3cccc4oc5ccccc5c34)cc2)cc1 ; c1ccc(-c2ccc(N(c3cccc(-c4cc5ccccc5c5c4ccc4ccc6ccccc6c45)c3)c3cccc4c3sc3ccccc34)cc2)cc1 ; CC1(C)c2ccccc2-c2c(N(c3ccc(-c4ccccc4)cc3)c3ccccc3-c3cc4ccc5ccc6ccccc6c5c4c4ccccc34)cccc21]

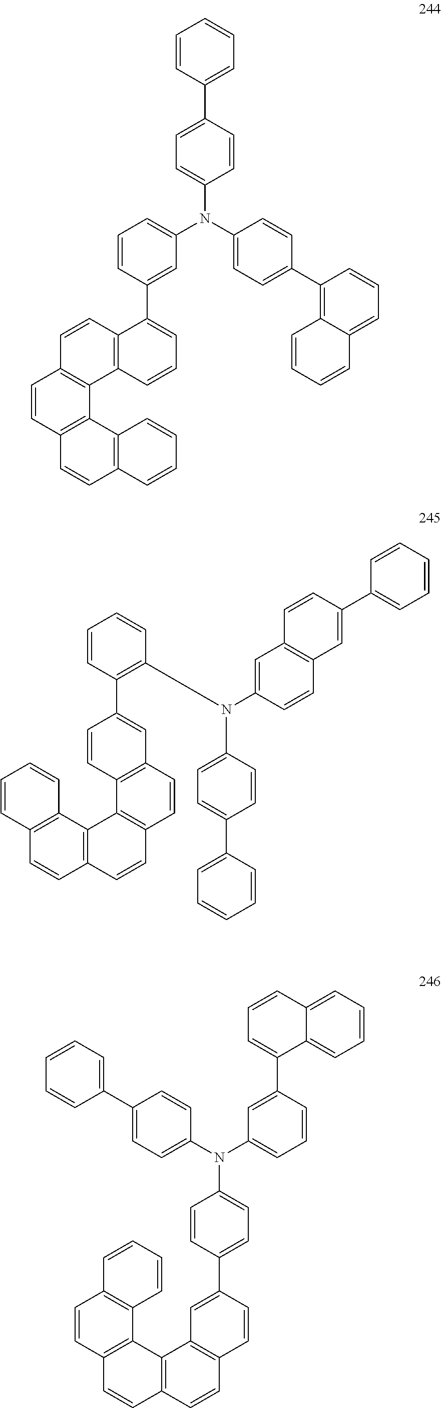 [SMILES: c1ccc(-c2ccc(N(c3ccc(-c4cccc5ccccc45)cc3)c3cccc(-c4cccc5c4ccc4ccc6ccc7ccccc7c6c45)c3)cc2)cc1 ; c1ccc(-c2ccc(N(c3ccc4cc(-c5ccccc5)ccc4c3)c3ccccc3-c3ccc4c(ccc5ccc6ccc7ccccc7c6c54)c3)cc2)cc1 ; c1ccc(-c2ccc(N(c3ccc(-c4ccc5ccc6ccc7ccc8ccccc8c7c6c5c4)cc3)c3cccc(-c4cccc5ccccc45)c3)cc2)cc1]

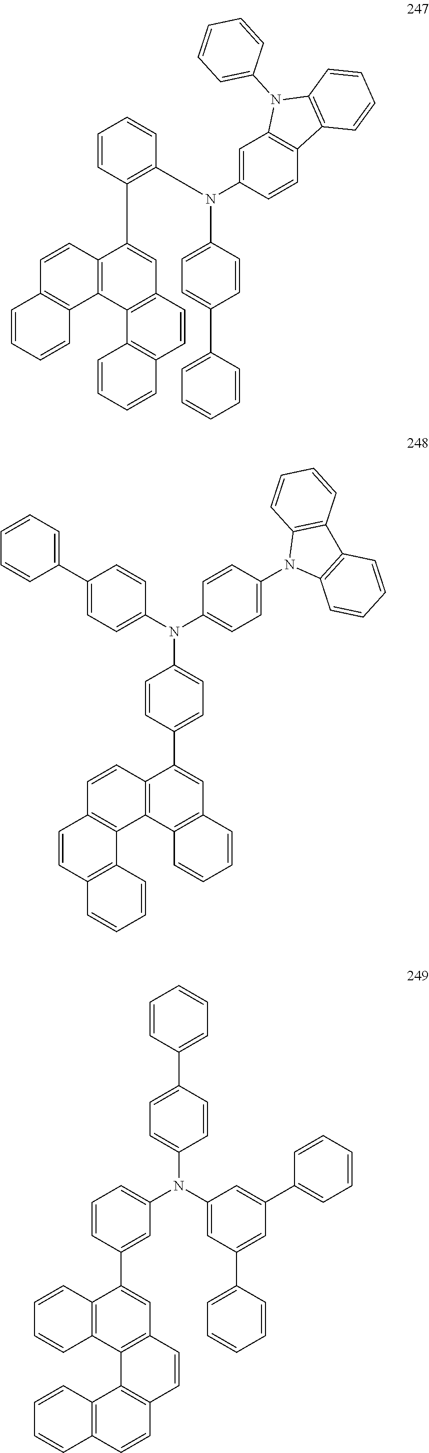 [SMILES: c1ccc(-c2ccc(N(c3ccc4c5ccccc5n(-c5ccccc5)c4c3)c3ccccc3-c3cc4ccc5ccccc5c4c4c3ccc3ccccc34)cc2)cc1 ; c1ccc(-c2ccc(N(c3ccc(-c4cc5ccccc5c5c4ccc4ccc6ccccc6c45)cc3)c3ccc(-n4c5ccccc5c5ccccc54)cc3)cc2)cc1 ; c1ccc(-c2ccc(N(c3cc(-c4ccccc4)cc(-c4ccccc4)c3)c3cccc(-c4cc5ccc6ccc7ccccc7c6c5c5ccccc45)c3)cc2)cc1]

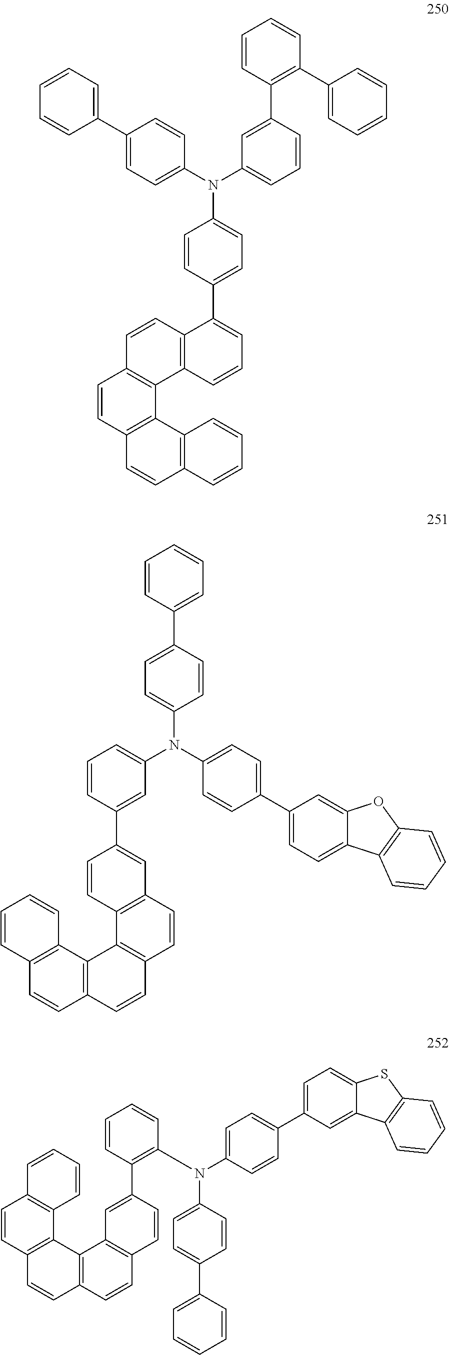 [SMILES: c1ccc(-c2ccc(N(c3ccc(-c4cccc5c4ccc4ccc6ccc7ccccc7c6c45)cc3)c3cccc(-c4ccccc4-c4ccccc4)c3)cc2)cc1 ; c1ccc(-c2ccc(N(c3ccc(-c4ccc5c(c4)oc4ccccc45)cc3)c3cccc(-c4ccc5c(ccc6ccc7ccc8ccccc8c7c65)c4)c3)cc2)cc1 ; c1ccc(-c2ccc(N(c3ccc(-c4ccc5sc6ccccc6c5c4)cc3)c3ccccc3-c3ccc4ccc5ccc6ccc7ccccc7c6c5c4c3)cc2)cc1]

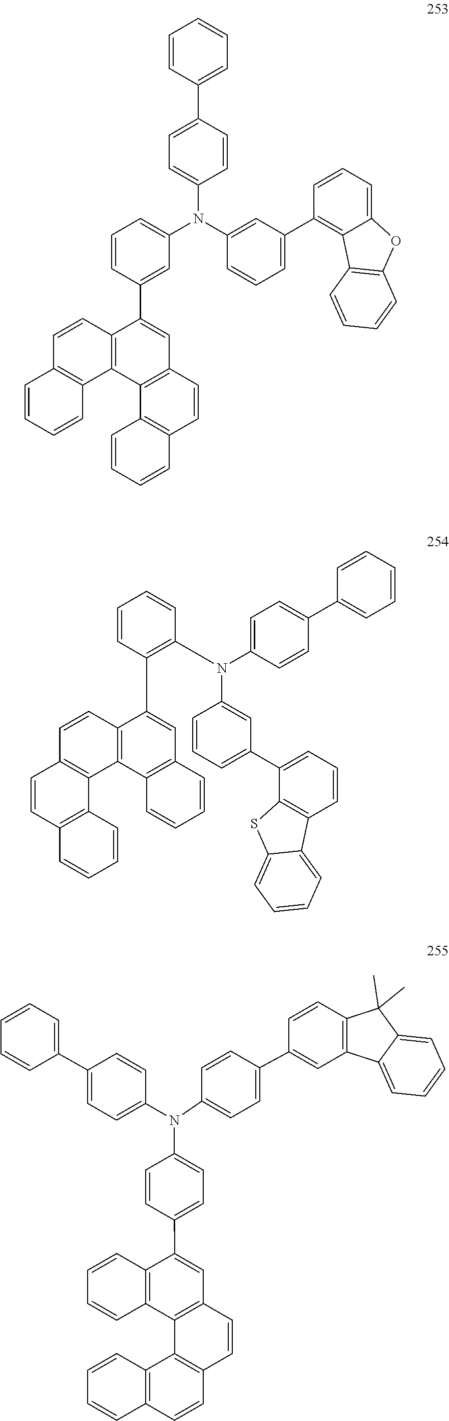 [SMILES: c1ccc(-c2ccc(N(c3cccc(-c4cc5ccc6ccccc6c5c5c4ccc4ccccc45)c3)c3cccc(-c4cccc5oc6ccccc6c45)c3)cc2)cc1 ; c1ccc(-c2ccc(N(c3cccc(-c4cccc5c4sc4ccccc45)c3)c3ccccc3-c3cc4ccccc4c4c3ccc3ccc5ccccc5c34)cc2)cc1 ; CC1(C)c2ccccc2-c2cc(-c3ccc(N(c4ccc(-c5ccccc5)cc4)c4ccc(-c5cc6ccc7ccc8ccccc8c7c6c6ccccc56)cc4)cc3)ccc21]

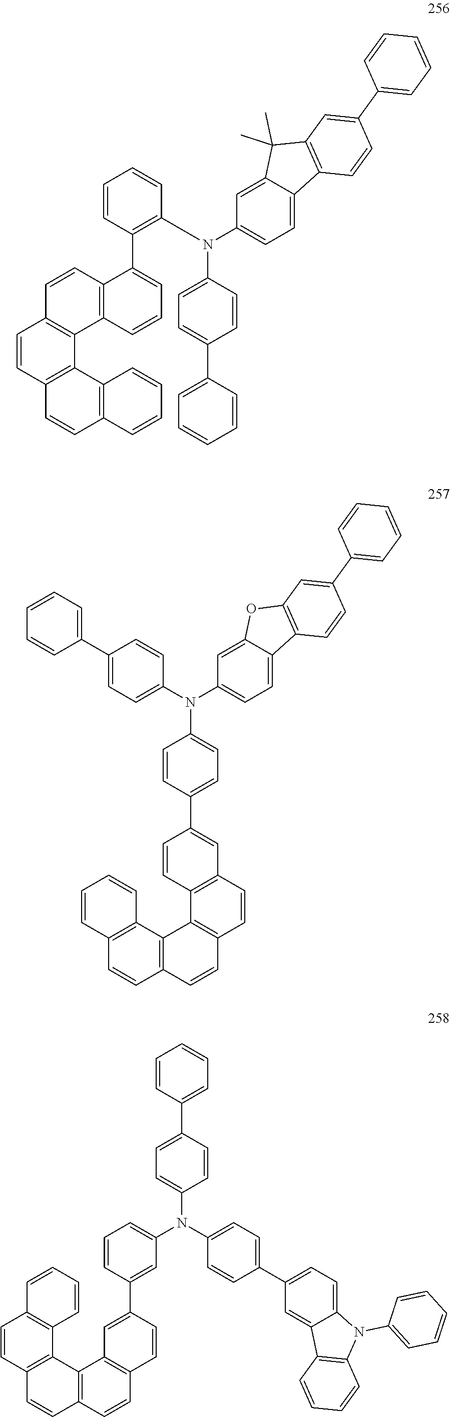 [SMILES: CC1(C)c2cc(-c3ccccc3)ccc2-c2ccc(N(c3ccc(-c4ccccc4)cc3)c3ccccc3-c3cccc4c3ccc3ccc5ccc6ccccc6c5c34)cc21 ; c1ccc(-c2ccc(N(c3ccc(-c4ccc5c(ccc6ccc7ccc8ccccc8c7c65)c4)cc3)c3ccc4c(c3)oc3cc(-c5ccccc5)ccc34)cc2)cc1 ; c1ccc(-c2ccc(N(c3ccc(-c4ccc5c(c4)c4ccccc4n5-c4ccccc4)cc3)c3cccc(-c4ccc5ccc6ccc7ccc8ccccc8c7c6c5c4)c3)cc2)cc1]

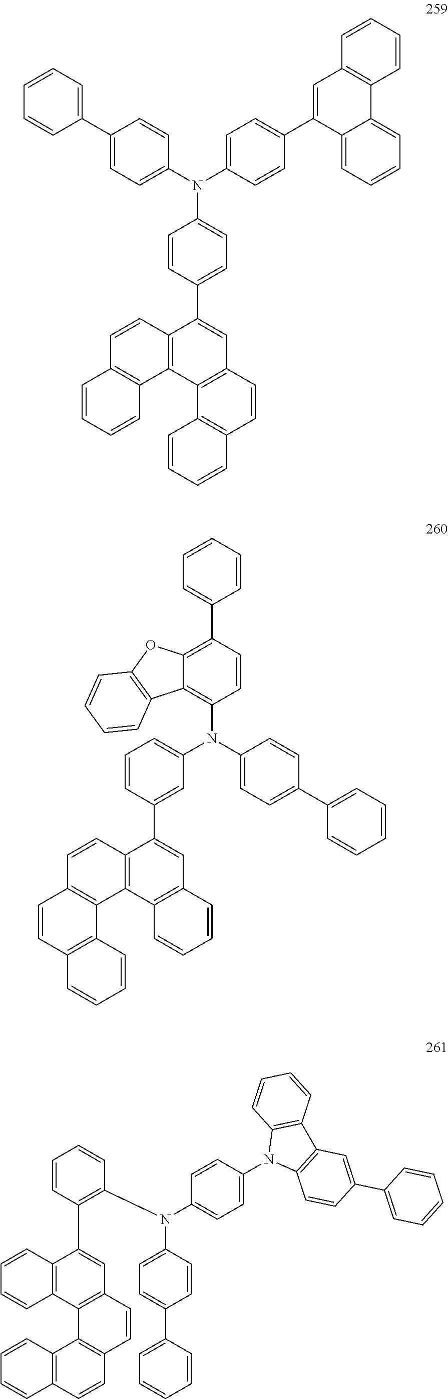 [SMILES: c1ccc(-c2ccc(N(c3ccc(-c4cc5ccccc5c5ccccc45)cc3)c3ccc(-c4cc5ccc6ccccc6c5c5c4ccc4ccccc45)cc3)cc2)cc1 ; c1ccc(-c2ccc(N(c3cccc(-c4cc5ccccc5c5c4ccc4ccc6ccccc6c45)c3)c3ccc(-c4ccccc4)c4oc5ccccc5c34)cc2)cc1 ; c1ccc(-c2ccc(N(c3ccc(-n4c5ccccc5c5cc(-c6ccccc6)ccc54)cc3)c3ccccc3-c3cc4ccc5ccc6ccccc6c5c4c4ccccc34)cc2)cc1]

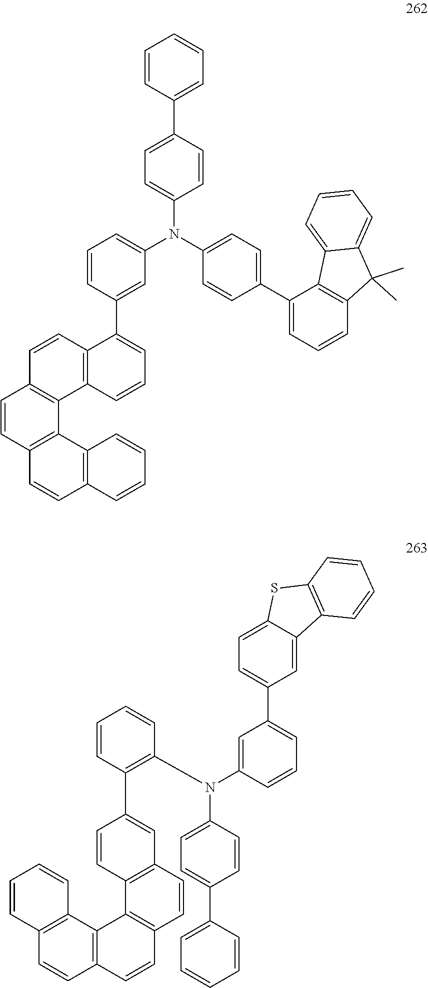 [SMILES: CC1(C)c2ccccc2-c2c(-c3ccc(N(c4ccc(-c5ccccc5)cc4)c4cccc(-c5cccc6c5ccc5ccc7ccc8ccccc8c7c56)c4)cc3)cccc21 ; c1ccc(-c2ccc(N(c3cccc(-c4ccc5sc6ccccc6c5c4)c3)c3ccccc3-c3ccc4c(ccc5ccc6ccc7ccccc7c6c54)c3)cc2)cc1]

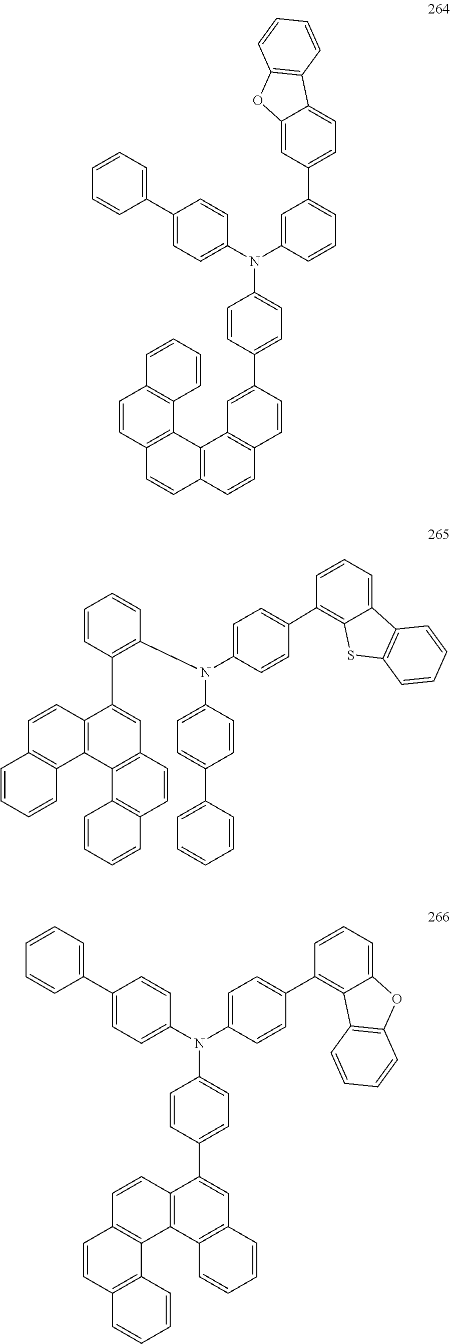 [SMILES: c1ccc(-c2ccc(N(c3ccc(-c4ccc5ccc6ccc7ccc8ccccc8c7c6c5c4)cc3)c3cccc(-c4ccc5c(c4)oc4ccccc45)c3)cc2)cc1 ; c1ccc(-c2ccc(N(c3ccc(-c4cccc5c4sc4ccccc45)cc3)c3ccccc3-c3cc4ccc5ccccc5c4c4c3ccc3ccccc34)cc2)cc1 ; c1ccc(-c2ccc(N(c3ccc(-c4cc5ccccc5c5c4ccc4ccc6ccccc6c45)cc3)c3ccc(-c4cccc5oc6ccccc6c45)cc3)cc2)cc1]

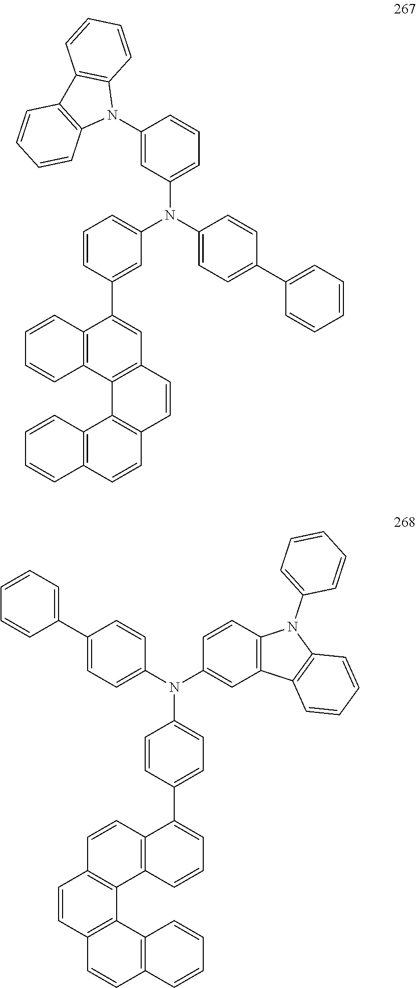 [SMILES: c1ccc(-c2ccc(N(c3cccc(-c4cc5ccc6ccc7ccccc7c6c5c5ccccc45)c3)c3cccc(-n4c5ccccc5c5ccccc54)c3)cc2)cc1 ; c1ccc(-c2ccc(N(c3ccc(-c4cccc5c4ccc4ccc6ccc7ccccc7c6c45)cc3)c3ccc4c(c3)c3ccccc3n4-c3ccccc3)cc2)cc1]

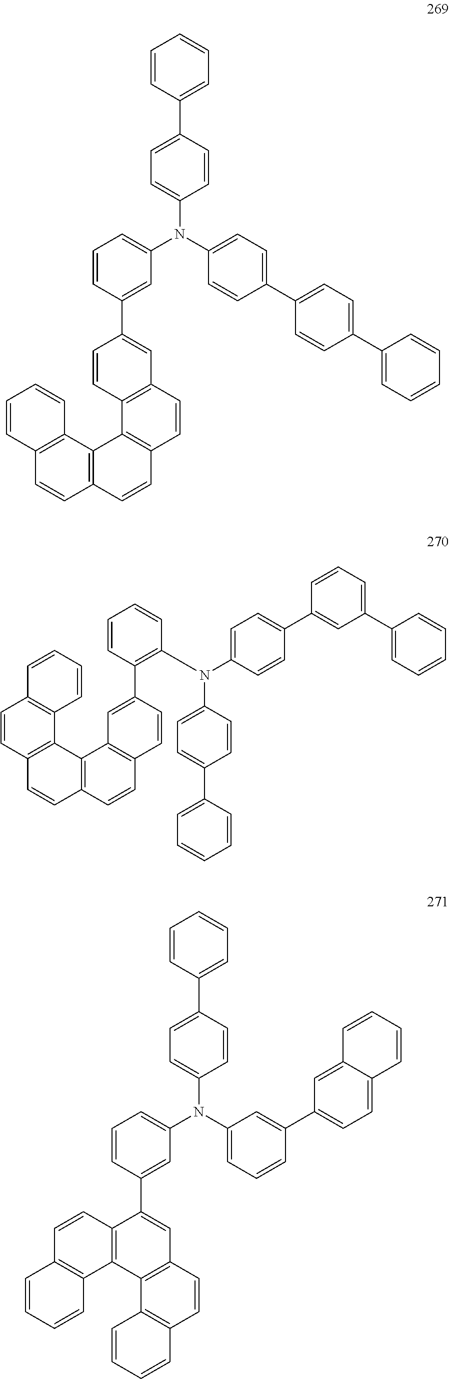 [SMILES: c1ccc(-c2ccc(-c3ccc(N(c4ccc(-c5ccccc5)cc4)c4cccc(-c5ccc6c(ccc7ccc8ccc9ccccc9c8c76)c5)c4)cc3)cc2)cc1 ; c1ccc(-c2ccc(N(c3ccc(-c4cccc(-c5ccccc5)c4)cc3)c3ccccc3-c3ccc4ccc5ccc6ccc7ccccc7c6c5c4c3)cc2)cc1 ; c1ccc(-c2ccc(N(c3cccc(-c4ccc5ccccc5c4)c3)c3cccc(-c4cc5ccc6ccccc6c5c5c4ccc4ccccc45)c3)cc2)cc1]

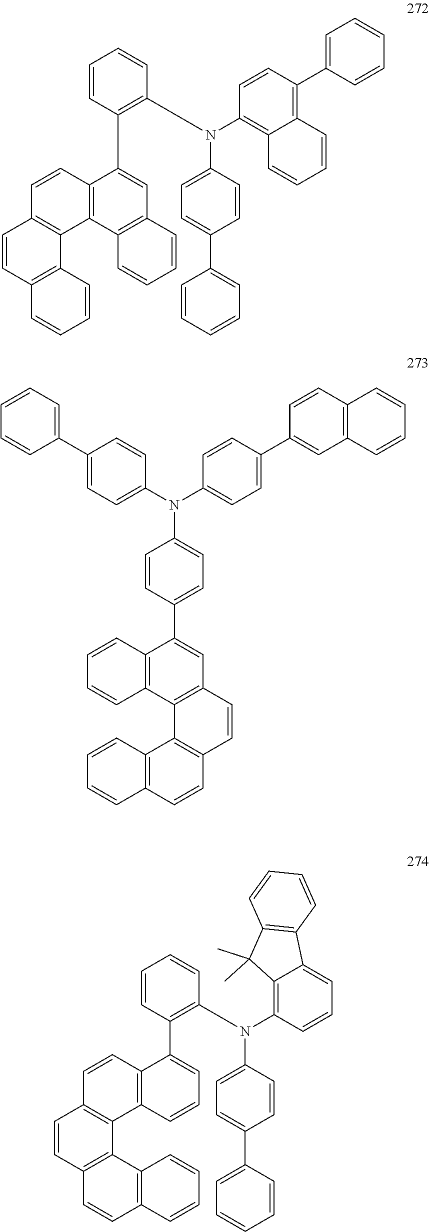 [SMILES: c1ccc(-c2ccc(N(c3ccccc3-c3cc4ccccc4c4c3ccc3ccc5ccccc5c34)c3ccc(-c4ccccc4)c4ccccc34)cc2)cc1 ; c1ccc(-c2ccc(N(c3ccc(-c4ccc5ccccc5c4)cc3)c3ccc(-c4cc5ccc6ccc7ccccc7c6c5c5ccccc45)cc3)cc2)cc1 ; CC1(C)c2ccccc2-c2cccc(N(c3ccc(-c4ccccc4)cc3)c3ccccc3-c3cccc4c3ccc3ccc5ccc6ccccc6c5c34)c21]

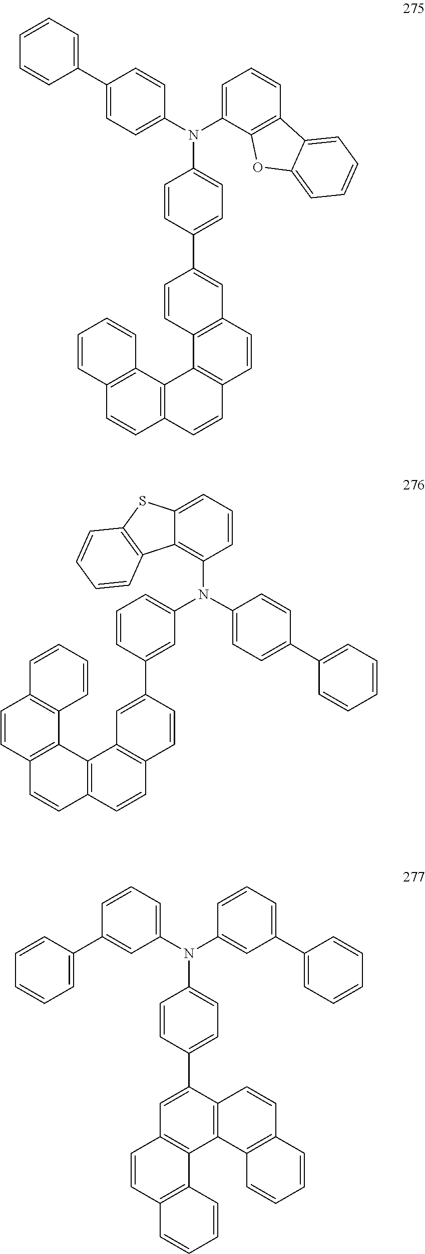 [SMILES: c1ccc(-c2ccc(N(c3ccc(-c4ccc5c(ccc6ccc7ccc8ccccc8c7c65)c4)cc3)c3cccc4c3oc3ccccc34)cc2)cc1 ; c1ccc(-c2ccc(N(c3cccc(-c4ccc5ccc6ccc7ccc8ccccc8c7c6c5c4)c3)c3cccc4sc5ccccc5c34)cc2)cc1 ; c1ccc(-c2cccc(N(c3ccc(-c4cc5ccc6ccccc6c5c5c4ccc4ccccc45)cc3)c3cccc(-c4ccccc4)c3)c2)cc1]

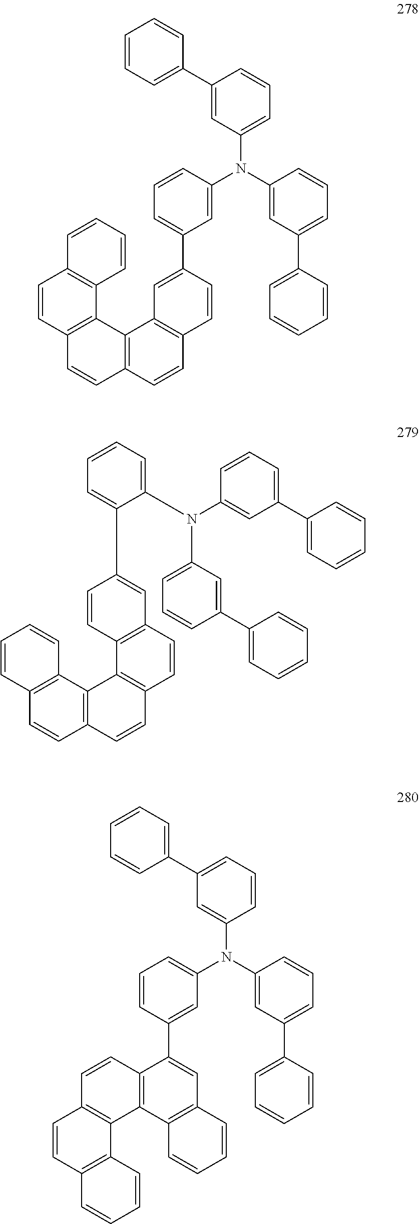 [SMILES: c1ccc(-c2cccc(N(c3cccc(-c4ccccc4)c3)c3cccc(-c4ccc5ccc6ccc7ccc8ccccc8c7c6c5c4)c3)c2)cc1 ; c1ccc(-c2cccc(N(c3cccc(-c4ccccc4)c3)c3ccccc3-c3ccc4c(ccc5ccc6ccc7ccccc7c6c54)c3)c2)cc1 ; c1ccc(-c2cccc(N(c3cccc(-c4ccccc4)c3)c3cccc(-c4cc5ccccc5c5c4ccc4ccc6ccccc6c45)c3)c2)cc1]

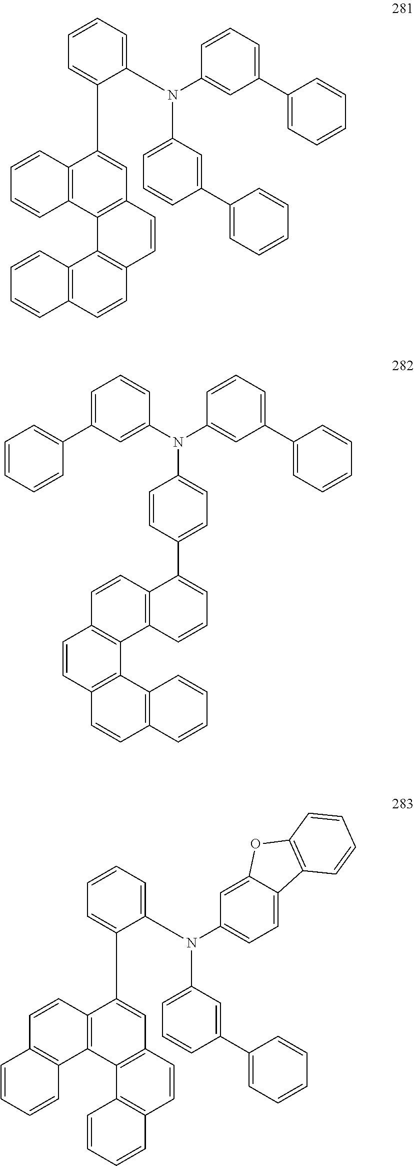 [SMILES: c1ccc(-c2cccc(N(c3cccc(-c4ccccc4)c3)c3ccccc3-c3cc4ccc5ccc6ccccc6c5c4c4ccccc34)c2)cc1 ; c1ccc(-c2cccc(N(c3ccc(-c4cccc5c4ccc4ccc6ccc7ccccc7c6c45)cc3)c3cccc(-c4ccccc4)c3)c2)cc1 ; c1ccc(-c2cccc(N(c3ccc4c(c3)oc3ccccc34)c3ccccc3-c3cc4ccc5ccccc5c4c4c3ccc3ccccc34)c2)cc1]

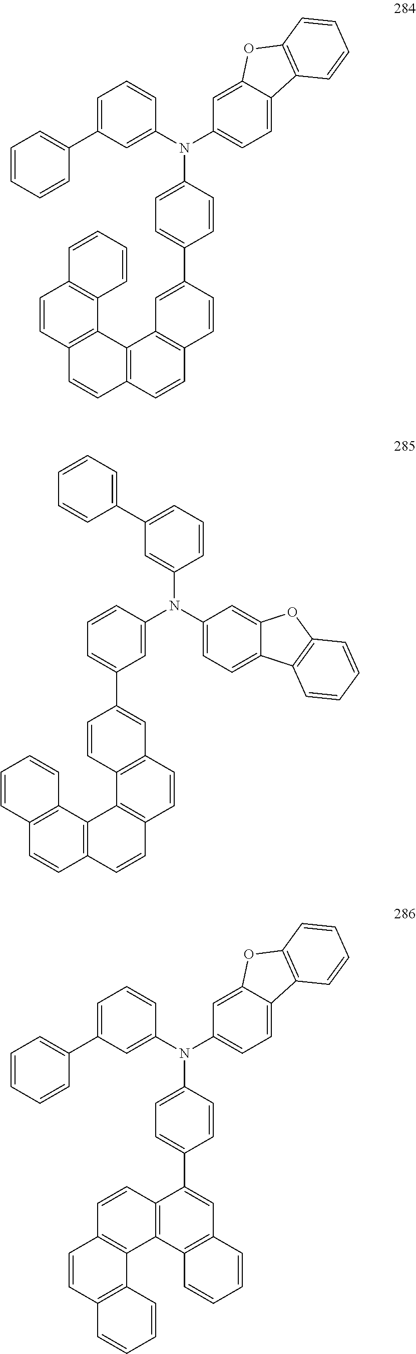 [SMILES: c1ccc(-c2cccc(N(c3ccc(-c4ccc5ccc6ccc7ccc8ccccc8c7c6c5c4)cc3)c3ccc4c(c3)oc3ccccc34)c2)cc1 ; c1ccc(-c2cccc(N(c3cccc(-c4ccc5c(ccc6ccc7ccc8ccccc8c7c65)c4)c3)c3ccc4c(c3)oc3ccccc34)c2)cc1 ; c1ccc(-c2cccc(N(c3ccc(-c4cc5ccccc5c5c4ccc4ccc6ccccc6c45)cc3)c3ccc4c(c3)oc3ccccc34)c2)cc1]

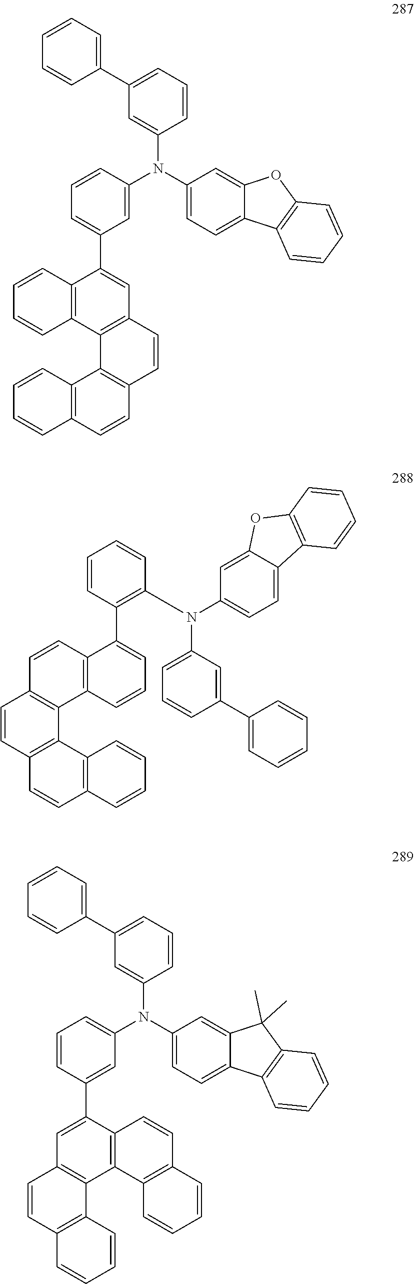 [SMILES: c1ccc(-c2cccc(N(c3cccc(-c4cc5ccc6ccc7ccccc7c6c5c5ccccc45)c3)c3ccc4c(c3)oc3ccccc34)c2)cc1 ; c1ccc(-c2cccc(N(c3ccc4c(c3)oc3ccccc34)c3ccccc3-c3cccc4c3ccc3ccc5ccc6ccccc6c5c34)c2)cc1 ; CC1(C)c2ccccc2-c2ccc(N(c3cccc(-c4ccccc4)c3)c3cccc(-c4cc5ccc6ccccc6c5c5c4ccc4ccccc45)c3)cc21]

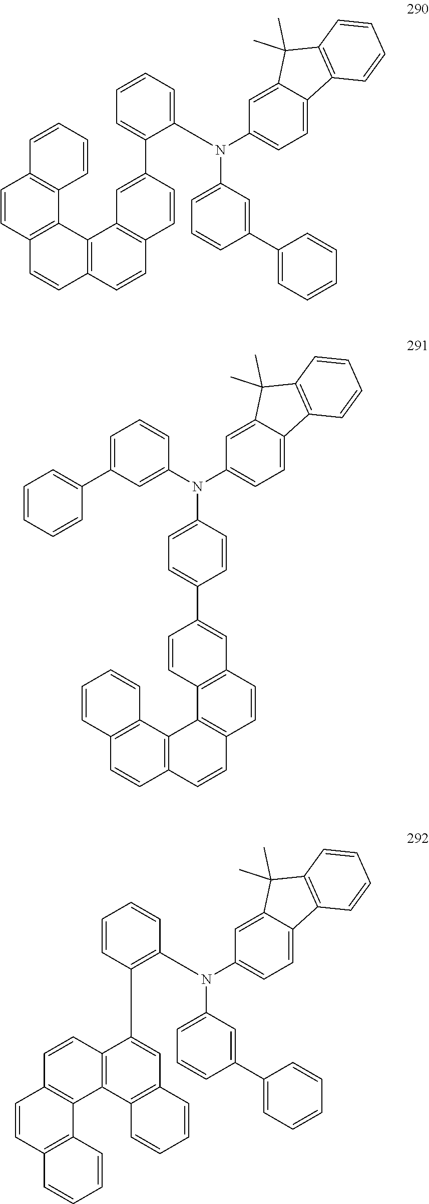 [SMILES: CC1(C)c2ccccc2-c2ccc(N(c3cccc(-c4ccccc4)c3)c3ccccc3-c3ccc4ccc5ccc6ccc7ccccc7c6c5c4c3)cc21 ; CC1(C)c2ccccc2-c2ccc(N(c3ccc(-c4ccc5c(ccc6ccc7ccc8ccccc8c7c65)c4)cc3)c3cccc(-c4ccccc4)c3)cc21 ; CC1(C)c2ccccc2-c2ccc(N(c3cccc(-c4ccccc4)c3)c3ccccc3-c3cc4ccccc4c4c3ccc3ccc5ccccc5c34)cc21]

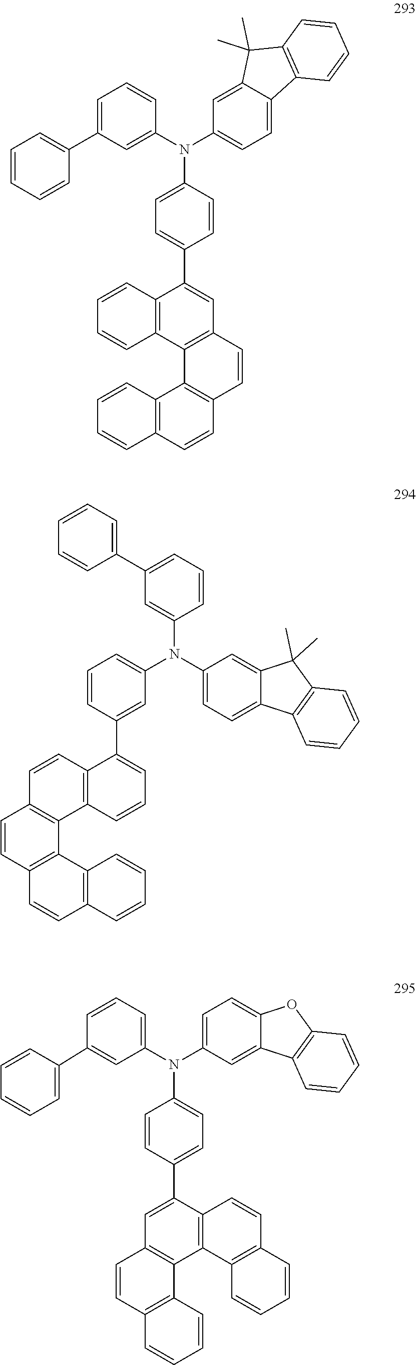 [SMILES: CC1(C)c2ccccc2-c2ccc(N(c3ccc(-c4cc5ccc6ccc7ccccc7c6c5c5ccccc45)cc3)c3cccc(-c4ccccc4)c3)cc21 ; CC1(C)c2ccccc2-c2ccc(N(c3cccc(-c4ccccc4)c3)c3cccc(-c4cccc5c4ccc4ccc6ccc7ccccc7c6c45)c3)cc21 ; c1ccc(-c2cccc(N(c3ccc(-c4cc5ccc6ccccc6c5c5c4ccc4ccccc45)cc3)c3ccc4oc5ccccc5c4c3)c2)cc1]

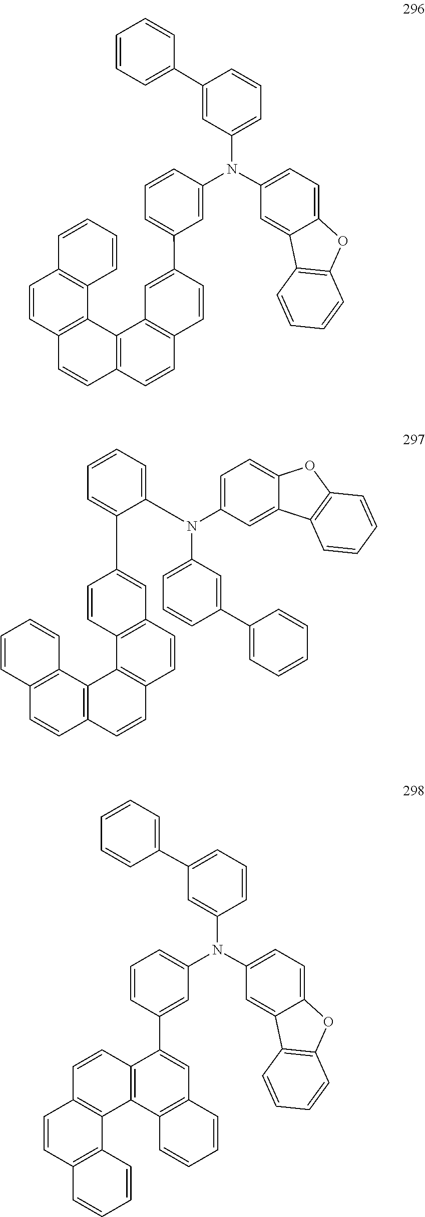 [SMILES: c1ccc(-c2cccc(N(c3cccc(-c4ccc5ccc6ccc7ccc8ccccc8c7c6c5c4)c3)c3ccc4oc5ccccc5c4c3)c2)cc1 ; c1ccc(-c2cccc(N(c3ccc4oc5ccccc5c4c3)c3ccccc3-c3ccc4c(ccc5ccc6ccc7ccccc7c6c54)c3)c2)cc1 ; c1ccc(-c2cccc(N(c3cccc(-c4cc5ccccc5c5c4ccc4ccc6ccccc6c45)c3)c3ccc4oc5ccccc5c4c3)c2)cc1]

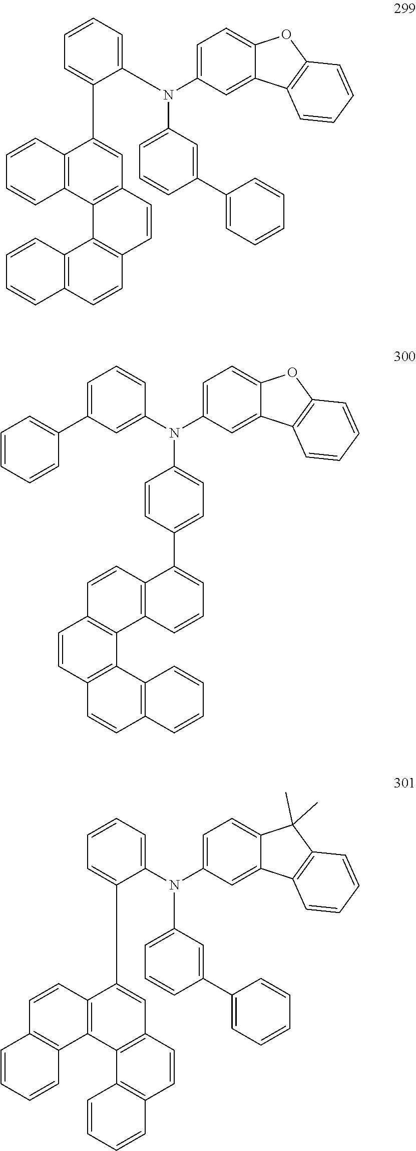 [SMILES: c1ccc(-c2cccc(N(c3ccc4oc5ccccc5c4c3)c3ccccc3-c3cc4ccc5ccc6ccccc6c5c4c4ccccc34)c2)cc1 ; c1ccc(-c2cccc(N(c3ccc(-c4cccc5c4ccc4ccc6ccc7ccccc7c6c45)cc3)c3ccc4oc5ccccc5c4c3)c2)cc1 ; CC1(C)c2ccccc2-c2cc(N(c3cccc(-c4ccccc4)c3)c3ccccc3-c3cc4ccc5ccccc5c4c4c3ccc3ccccc34)ccc21]

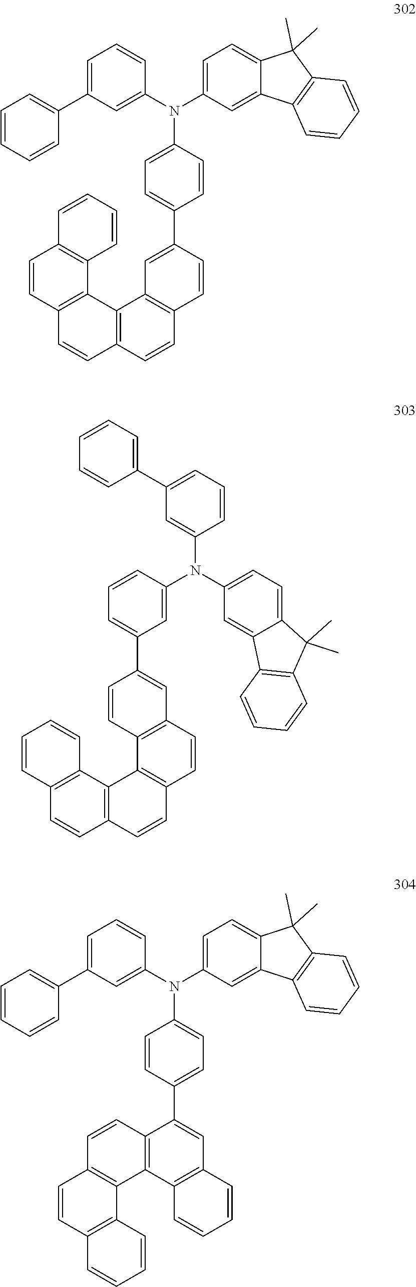 [SMILES: CC1(C)c2ccccc2-c2cc(N(c3ccc(-c4ccc5ccc6ccc7ccc8ccccc8c7c6c5c4)cc3)c3cccc(-c4ccccc4)c3)ccc21 ; CC1(C)c2ccccc2-c2cc(N(c3cccc(-c4ccccc4)c3)c3cccc(-c4ccc5c(ccc6ccc7ccc8ccccc8c7c65)c4)c3)ccc21 ; CC1(C)c2ccccc2-c2cc(N(c3ccc(-c4cc5ccccc5c5c4ccc4ccc6ccccc6c45)cc3)c3cccc(-c4ccccc4)c3)ccc21]

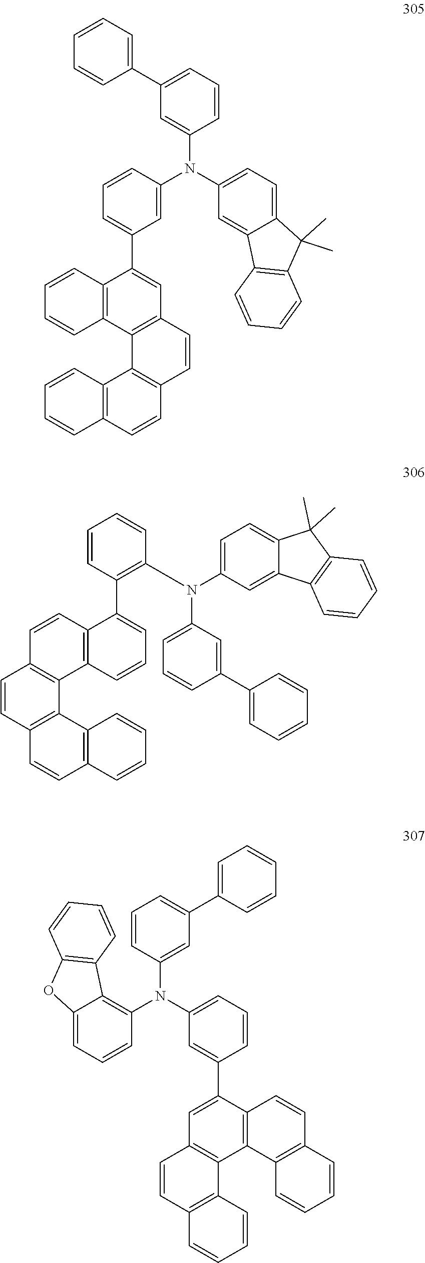 [SMILES: CC1(C)c2ccccc2-c2cc(N(c3cccc(-c4ccccc4)c3)c3cccc(-c4cc5ccc6ccc7ccccc7c6c5c5ccccc45)c3)ccc21 ; CC1(C)c2ccccc2-c2cc(N(c3cccc(-c4ccccc4)c3)c3ccccc3-c3cccc4c3ccc3ccc5ccc6ccccc6c5c34)ccc21 ; c1ccc(-c2cccc(N(c3cccc(-c4cc5ccc6ccccc6c5c5c4ccc4ccccc45)c3)c3cccc4oc5ccccc5c34)c2)cc1]

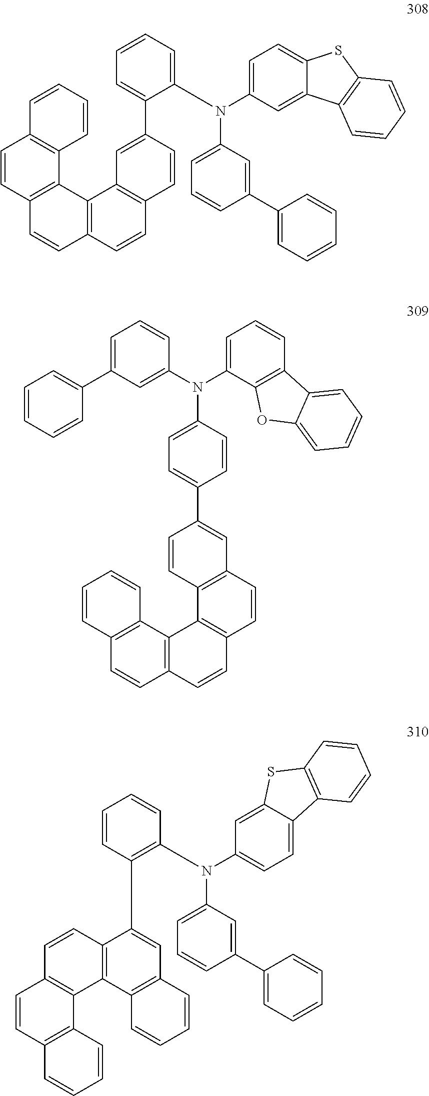 [SMILES: c1ccc(-c2cccc(N(c3ccc4sc5ccccc5c4c3)c3ccccc3-c3ccc4ccc5ccc6ccc7ccccc7c6c5c4c3)c2)cc1 ; c1ccc(-c2cccc(N(c3ccc(-c4ccc5c(ccc6ccc7ccc8ccccc8c7c65)c4)cc3)c3cccc4c3oc3ccccc34)c2)cc1 ; c1ccc(-c2cccc(N(c3ccc4c(c3)sc3ccccc34)c3ccccc3-c3cc4ccccc4c4c3ccc3ccc5ccccc5c34)c2)cc1]

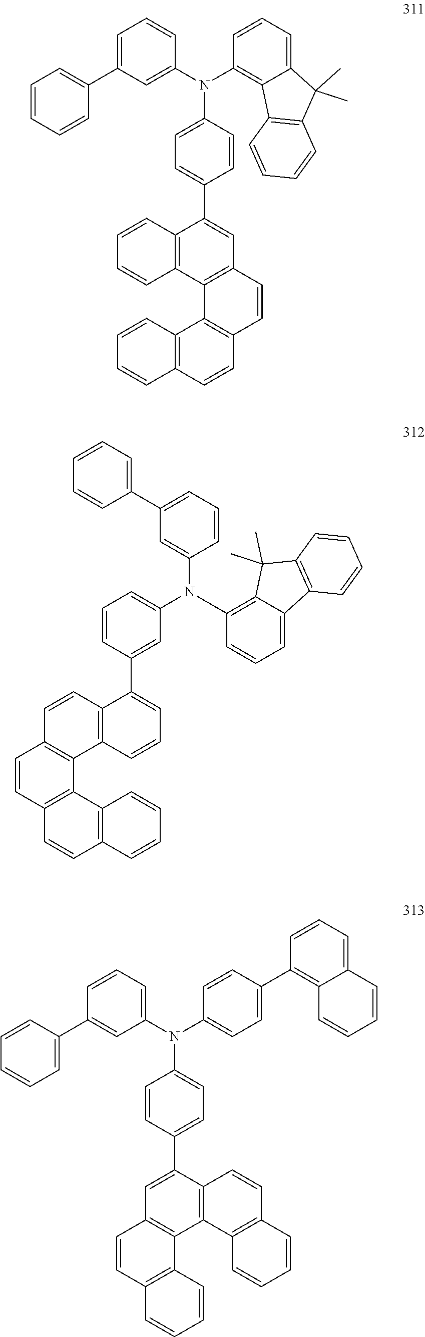 [SMILES: CC1(C)c2ccccc2-c2c(N(c3ccc(-c4cc5ccc6ccc7ccccc7c6c5c5ccccc45)cc3)c3cccc(-c4ccccc4)c3)cccc21 ; CC1(C)c2ccccc2-c2cccc(N(c3cccc(-c4ccccc4)c3)c3cccc(-c4cccc5c4ccc4ccc6ccc7ccccc7c6c45)c3)c21 ; c1ccc(-c2cccc(N(c3ccc(-c4cccc5ccccc45)cc3)c3ccc(-c4cc5ccc6ccccc6c5c5c4ccc4ccccc45)cc3)c2)cc1]

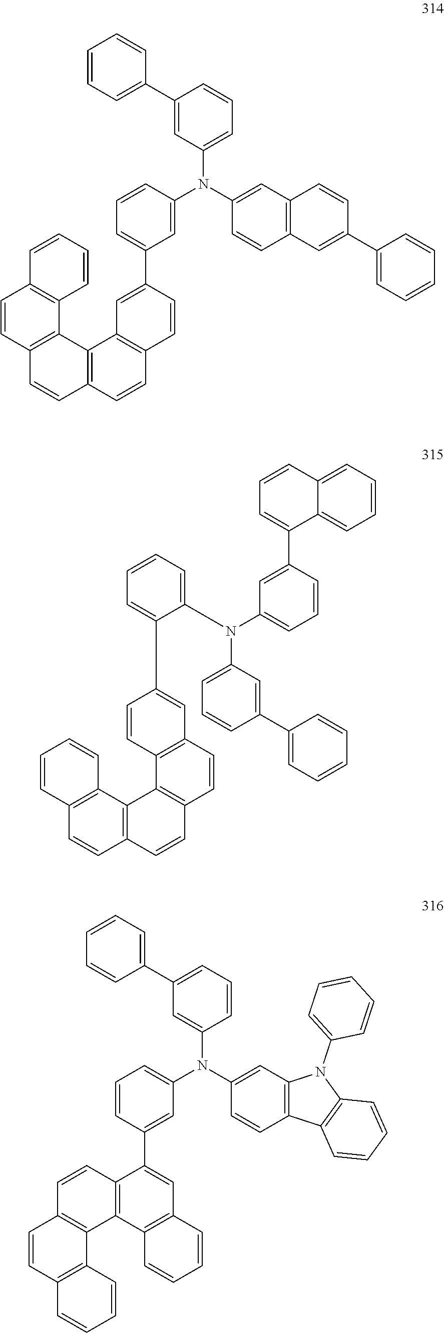 [SMILES: c1ccc(-c2cccc(N(c3cccc(-c4ccc5ccc6ccc7ccc8ccccc8c7c6c5c4)c3)c3ccc4cc(-c5ccccc5)ccc4c3)c2)cc1 ; c1ccc(-c2cccc(N(c3cccc(-c4cccc5ccccc45)c3)c3ccccc3-c3ccc4c(ccc5ccc6ccc7ccccc7c6c54)c3)c2)cc1 ; c1ccc(-c2cccc(N(c3cccc(-c4cc5ccccc5c5c4ccc4ccc6ccccc6c45)c3)c3ccc4c5ccccc5n(-c5ccccc5)c4c3)c2)cc1]

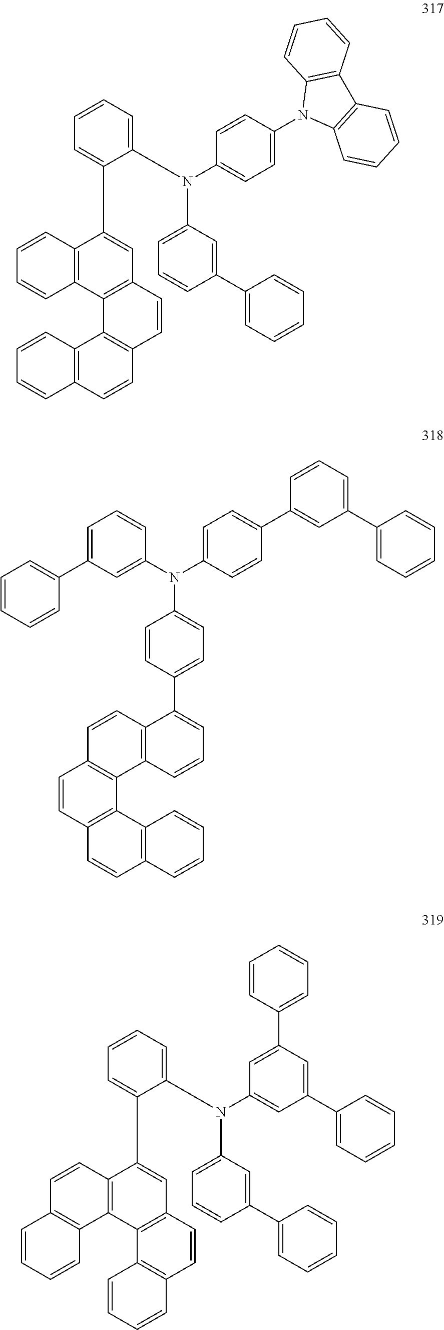 [SMILES: c1ccc(-c2cccc(N(c3ccc(-n4c5ccccc5c5ccccc54)cc3)c3ccccc3-c3cc4ccc5ccc6ccccc6c5c4c4ccccc34)c2)cc1 ; c1ccc(-c2cccc(-c3ccc(N(c4ccc(-c5cccc6c5ccc5ccc7ccc8ccccc8c7c56)cc4)c4cccc(-c5ccccc5)c4)cc3)c2)cc1 ; c1ccc(-c2cccc(N(c3cc(-c4ccccc4)cc(-c4ccccc4)c3)c3ccccc3-c3cc4ccc5ccccc5c4c4c3ccc3ccccc34)c2)cc1]

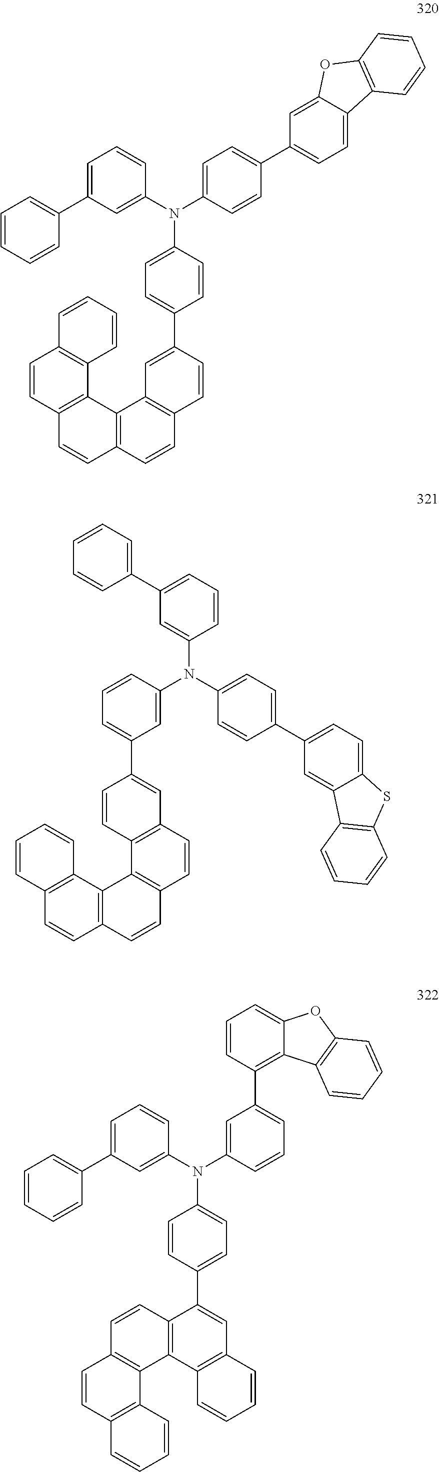 [SMILES: c1ccc(-c2cccc(N(c3ccc(-c4ccc5c(c4)oc4ccccc45)cc3)c3ccc(-c4ccc5ccc6ccc7ccc8ccccc8c7c6c5c4)cc3)c2)cc1 ; c1ccc(-c2cccc(N(c3ccc(-c4ccc5sc6ccccc6c5c4)cc3)c3cccc(-c4ccc5c(ccc6ccc7ccc8ccccc8c7c65)c4)c3)c2)cc1 ; c1ccc(-c2cccc(N(c3ccc(-c4cc5ccccc5c5c4ccc4ccc6ccccc6c45)cc3)c3cccc(-c4cccc5oc6ccccc6c45)c3)c2)cc1]

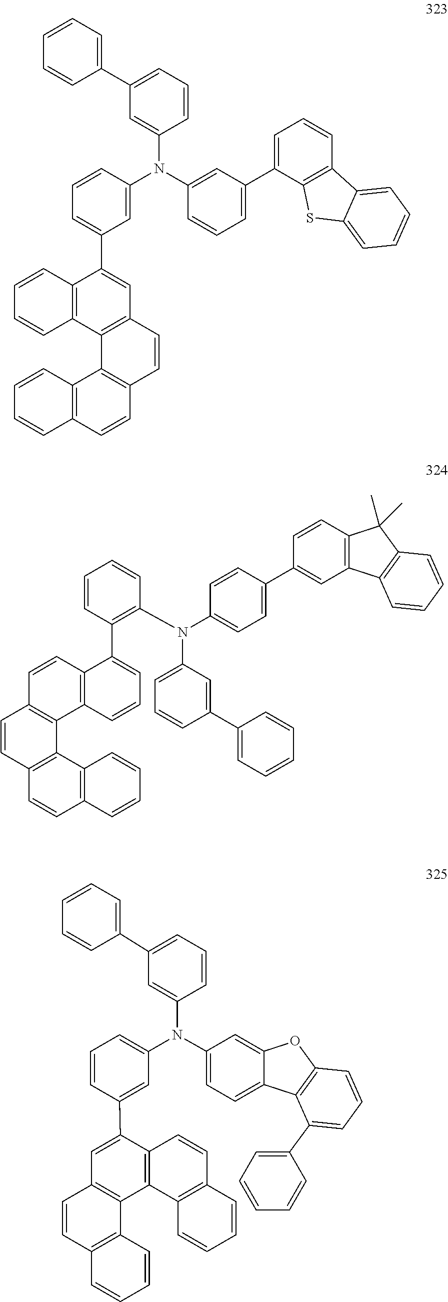 [SMILES: c1ccc(-c2cccc(N(c3cccc(-c4cc5ccc6ccc7ccccc7c6c5c5ccccc45)c3)c3cccc(-c4cccc5c4sc4ccccc45)c3)c2)cc1 ; CC1(C)c2ccccc2-c2cc(-c3ccc(N(c4cccc(-c5ccccc5)c4)c4ccccc4-c4cccc5c4ccc4ccc6ccc7ccccc7c6c45)cc3)ccc21 ; c1ccc(-c2cccc(N(c3cccc(-c4cc5ccc6ccccc6c5c5c4ccc4ccccc45)c3)c3ccc4c(c3)oc3cccc(-c5ccccc5)c34)c2)cc1]

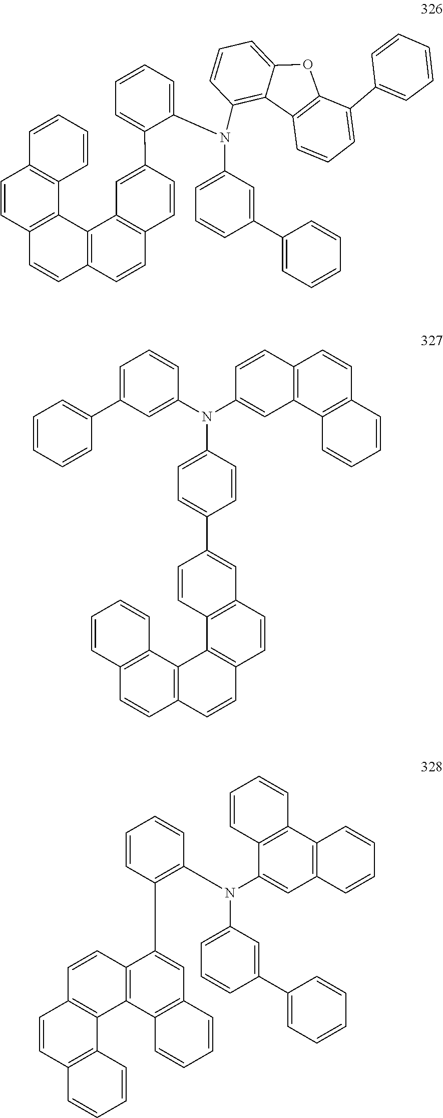 [SMILES: c1ccc(-c2cccc(N(c3ccccc3-c3ccc4ccc5ccc6ccc7ccccc7c6c5c4c3)c3cccc4oc5c(-c6ccccc6)cccc5c34)c2)cc1 ; c1ccc(-c2cccc(N(c3ccc(-c4ccc5c(ccc6ccc7ccc8ccccc8c7c65)c4)cc3)c3ccc4ccc5ccccc5c4c3)c2)cc1 ; c1ccc(-c2cccc(N(c3ccccc3-c3cc4ccccc4c4c3ccc3ccc5ccccc5c34)c3cc4ccccc4c4ccccc34)c2)cc1]

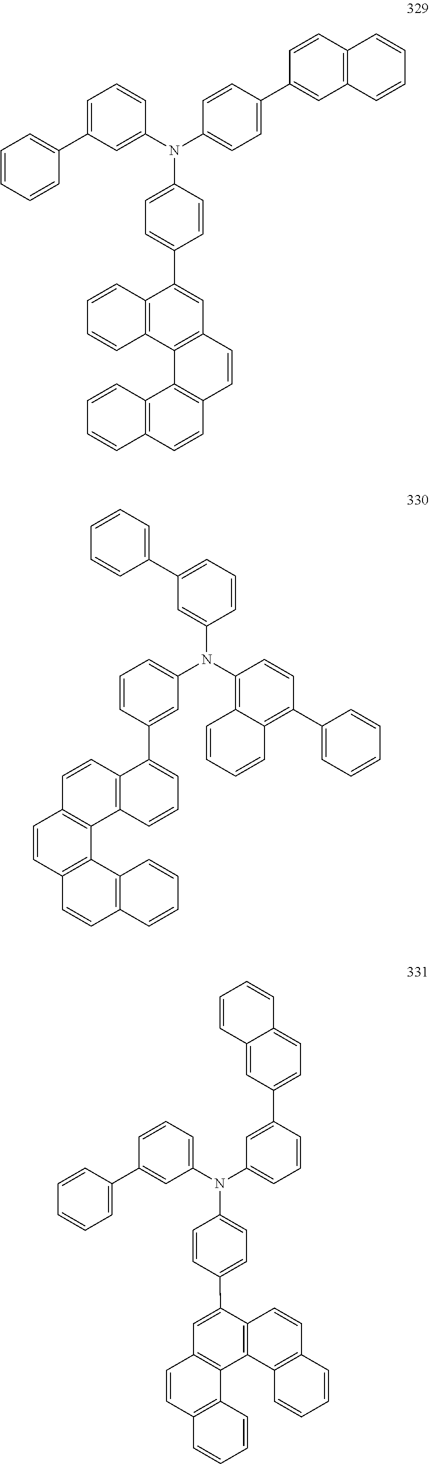

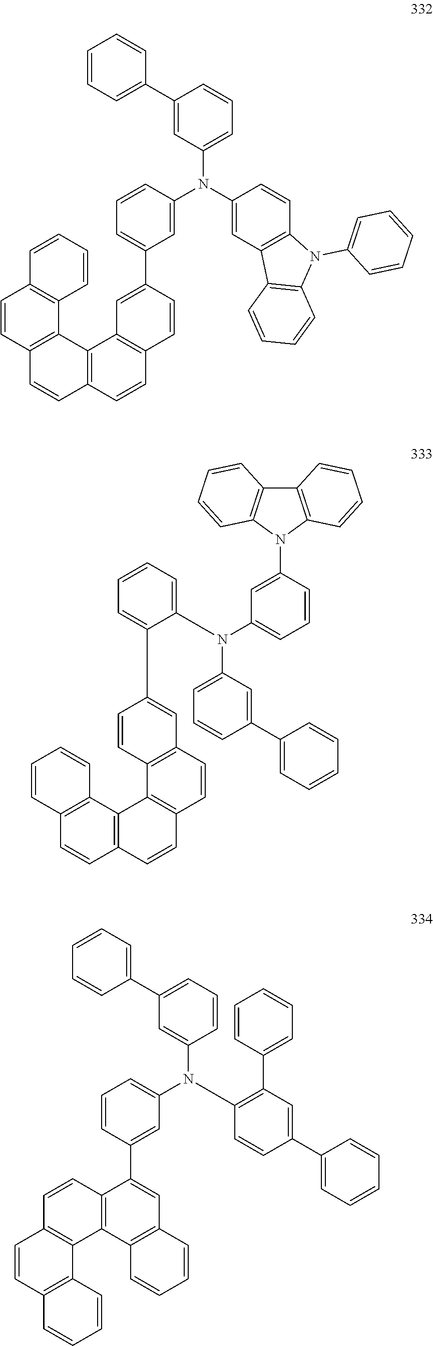 [SMILES: c1ccc(-c2cccc(N(c3cccc(-c4ccc5ccc6ccc7ccc8ccccc8c7c6c5c4)c3)c3ccc4c(c3)c3ccccc3n4-c3ccccc3)c2)cc1 ; c1ccc(-c2cccc(N(c3cccc(-n4c5ccccc5c5ccccc54)c3)c3ccccc3-c3ccc4c(ccc5ccc6ccc7ccccc7c6c54)c3)c2)cc1 ; c1ccc(-c2cccc(N(c3cccc(-c4cc5ccccc5c5c4ccc4ccc6ccccc6c45)c3)c3ccc(-c4ccccc4)cc3-c3ccccc3)c2)cc1]

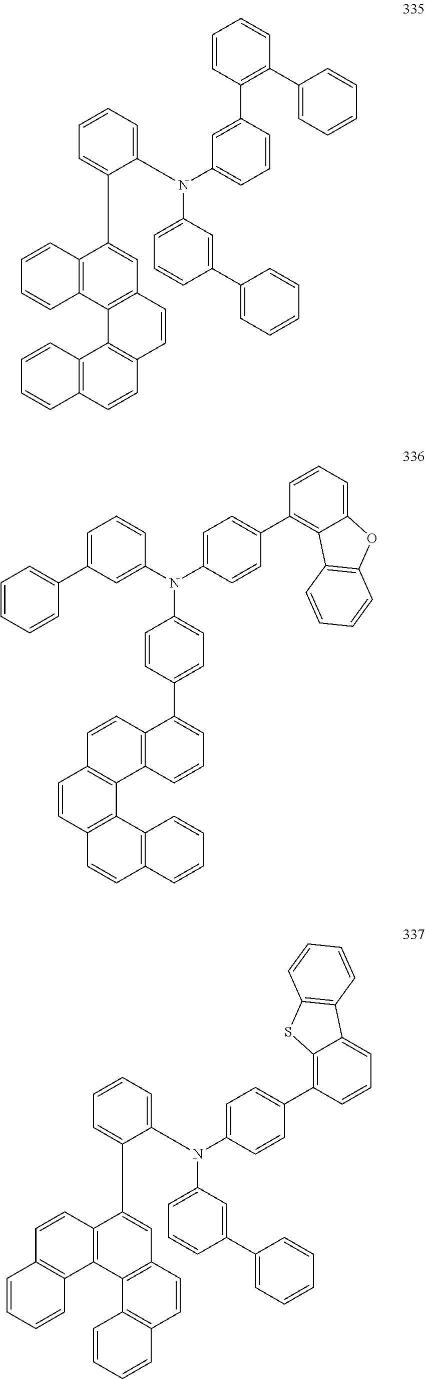 [SMILES: c1ccc(-c2cccc(N(c3cccc(-c4ccccc4-c4ccccc4)c3)c3ccccc3-c3cc4ccc5ccc6ccccc6c5c4c4ccccc34)c2)cc1 ; c1ccc(-c2cccc(N(c3ccc(-c4cccc5c4ccc4ccc6ccc7ccccc7c6c45)cc3)c3ccc(-c4cccc5oc6ccccc6c45)cc3)c2)cc1 ; c1ccc(-c2cccc(N(c3ccc(-c4cccc5c4sc4ccccc45)cc3)c3ccccc3-c3cc4ccc5ccccc5c4c4c3ccc3ccccc34)c2)cc1]

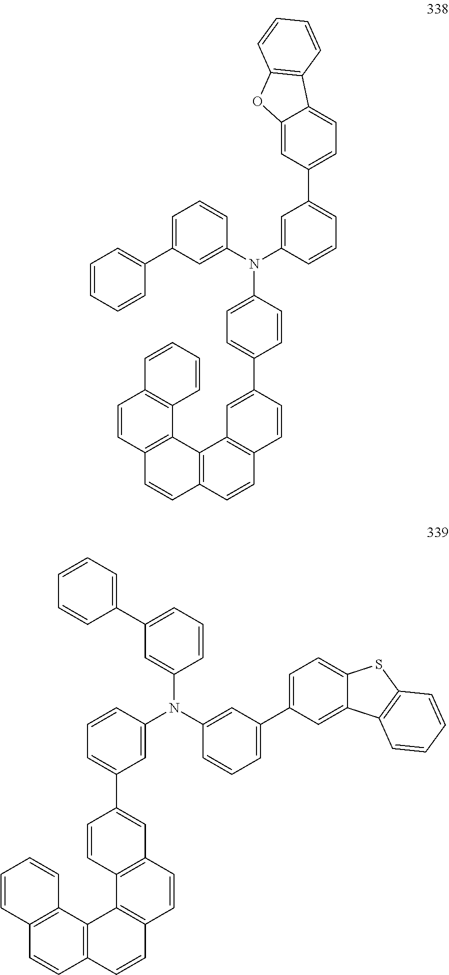 [SMILES: c1ccc(-c2cccc(N(c3ccc(-c4ccc5ccc6ccc7ccc8ccccc8c7c6c5c4)cc3)c3cccc(-c4ccc5c(c4)oc4ccccc45)c3)c2)cc1 ; c1ccc(-c2cccc(N(c3cccc(-c4ccc5c(ccc6ccc7ccc8ccccc8c7c65)c4)c3)c3cccc(-c4ccc5sc6ccccc6c5c4)c3)c2)cc1]

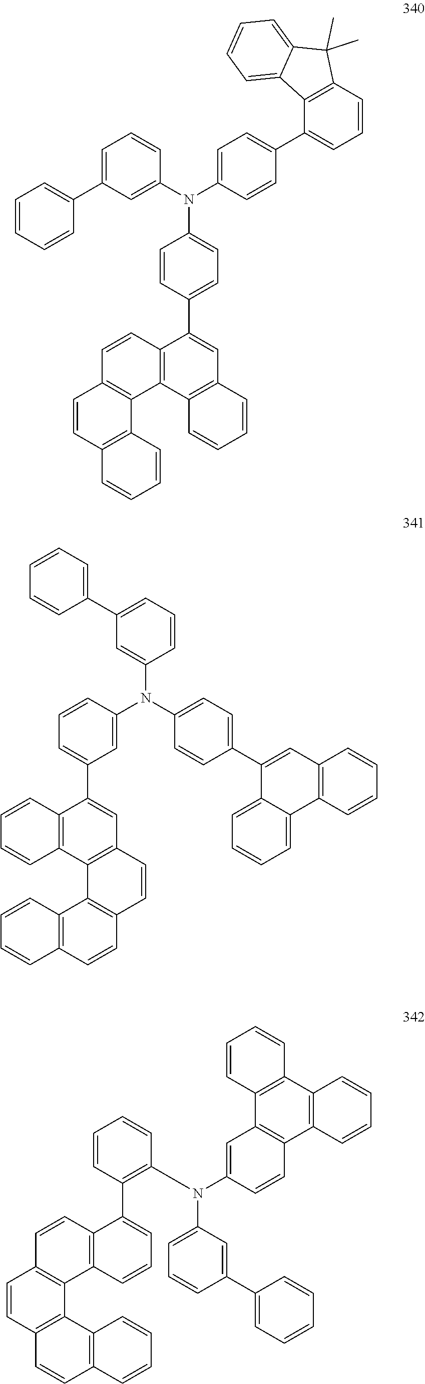 [SMILES: CC1(C)c2ccccc2-c2c(-c3ccc(N(c4ccc(-c5cc6ccccc6c6c5ccc5ccc7ccccc7c56)cc4)c4cccc(-c5ccccc5)c4)cc3)cccc21 ; c1ccc(-c2cccc(N(c3ccc(-c4cc5ccccc5c5ccccc45)cc3)c3cccc(-c4cc5ccc6ccc7ccccc7c6c5c5ccccc45)c3)c2)cc1 ; c1ccc(-c2cccc(N(c3ccc4c5ccccc5c5ccccc5c4c3)c3ccccc3-c3cccc4c3ccc3ccc5ccc6ccccc6c5c34)c2)cc1]

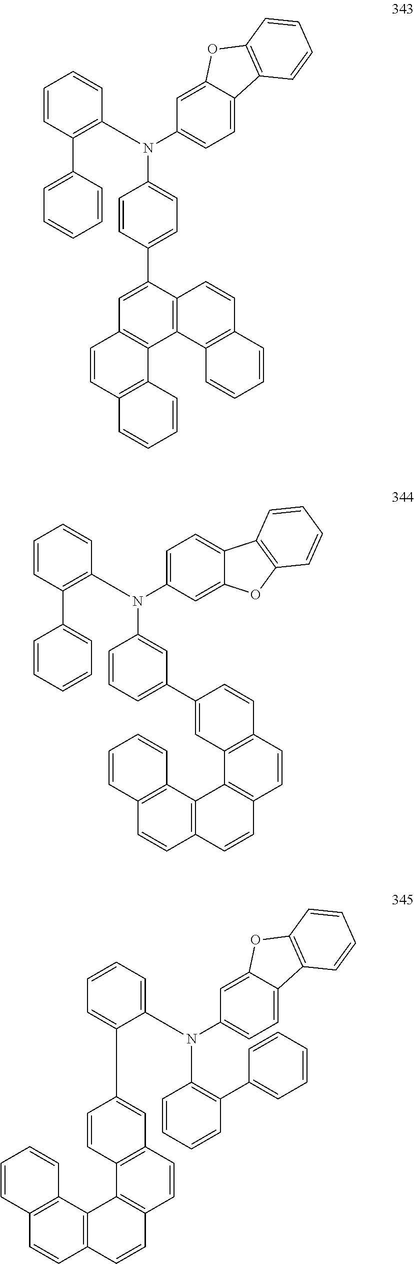 [SMILES: c1ccc(-c2ccccc2N(c2ccc(-c3cc4ccc5ccccc5c4c4c3ccc3ccccc34)cc2)c2ccc3c(c2)oc2ccccc23)cc1 ; c1ccc(-c2ccccc2N(c2cccc(-c3ccc4ccc5ccc6ccc7ccccc7c6c5c4c3)c2)c2ccc3c(c2)oc2ccccc23)cc1 ; c1ccc(-c2ccccc2N(c2ccc3c(c2)oc2ccccc23)c2ccccc2-c2ccc3c(ccc4ccc5ccc6ccccc6c5c43)c2)cc1]

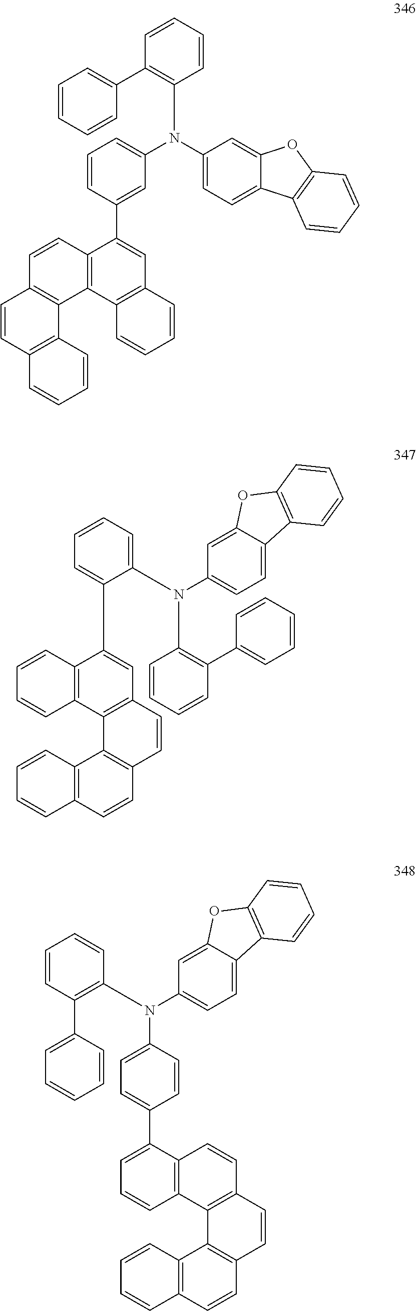 [SMILES: c1ccc(-c2ccccc2N(c2cccc(-c3cc4ccccc4c4c3ccc3ccc5ccccc5c34)c2)c2ccc3c(c2)oc2ccccc23)cc1 ; c1ccc(-c2ccccc2N(c2ccc3c(c2)oc2ccccc23)c2ccccc2-c2cc3ccc4ccc5ccccc5c4c3c3ccccc23)cc1 ; c1ccc(-c2ccccc2N(c2ccc(-c3cccc4c3ccc3ccc5ccc6ccccc6c5c34)cc2)c2ccc3c(c2)oc2ccccc23)cc1]

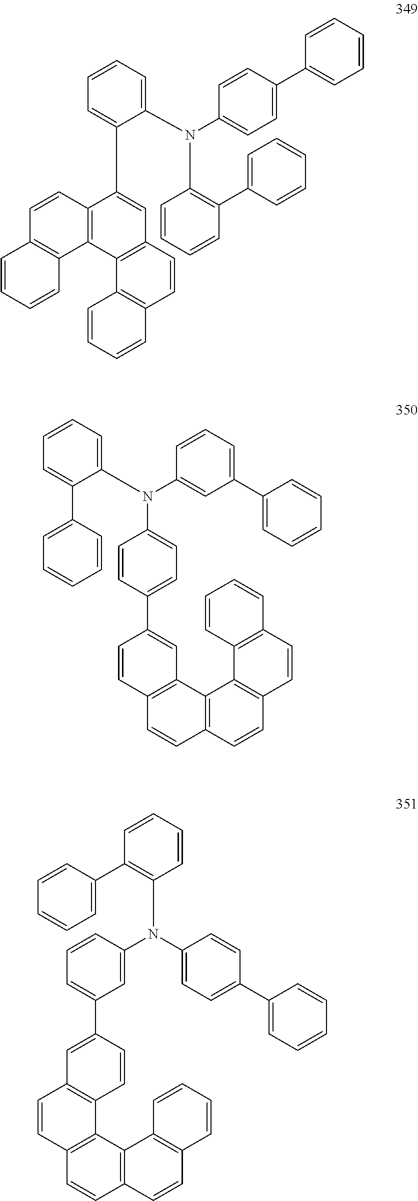 [SMILES: c1ccc(-c2ccc(N(c3ccccc3-c3ccccc3)c3ccccc3-c3cc4ccc5ccccc5c4c4c3ccc3ccccc34)cc2)cc1 ; c1ccc(-c2cccc(N(c3ccc(-c4ccc5ccc6ccc7ccc8ccccc8c7c6c5c4)cc3)c3ccccc3-c3ccccc3)c2)cc1 ; c1ccc(-c2ccc(N(c3cccc(-c4ccc5c(ccc6ccc7ccc8ccccc8c7c65)c4)c3)c3ccccc3-c3ccccc3)cc2)cc1]

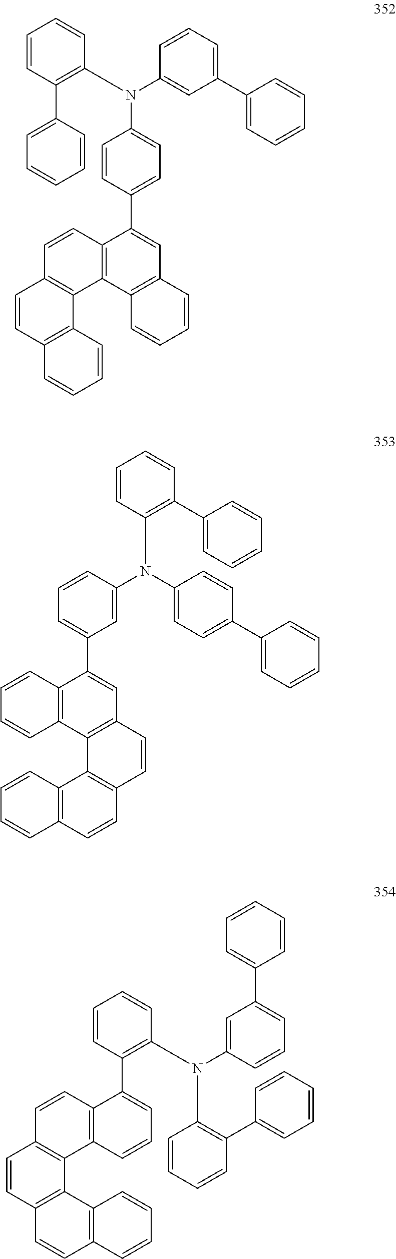 [SMILES: c1ccc(-c2cccc(N(c3ccc(-c4cc5ccccc5c5c4ccc4ccc6ccccc6c45)cc3)c3ccccc3-c3ccccc3)c2)cc1 ; c1ccc(-c2ccc(N(c3cccc(-c4cc5ccc6ccc7ccccc7c6c5c5ccccc45)c3)c3ccccc3-c3ccccc3)cc2)cc1 ; c1ccc(-c2cccc(N(c3ccccc3-c3ccccc3)c3ccccc3-c3cccc4c3ccc3ccc5ccc6ccccc6c5c34)c2)cc1]

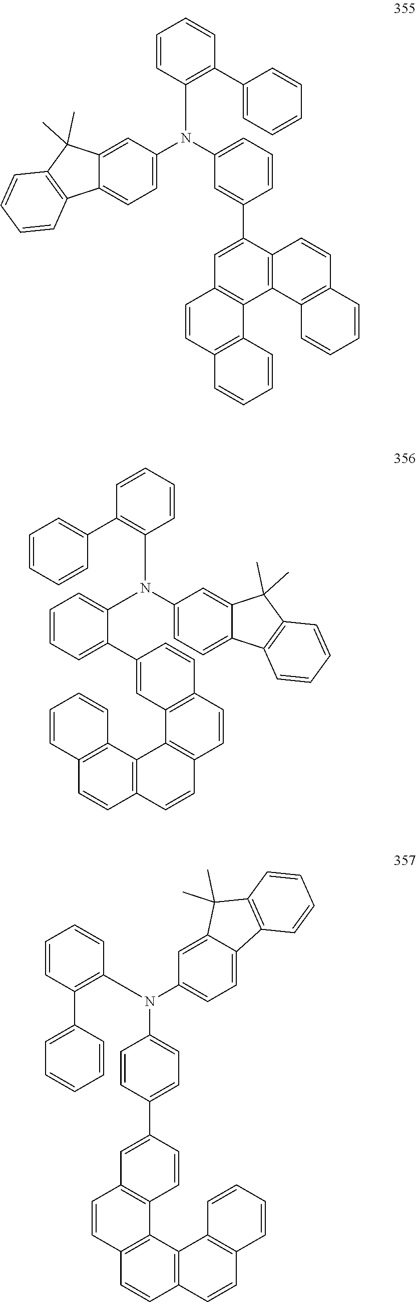 [SMILES: CC1(C)c2ccccc2-c2ccc(N(c3cccc(-c4cc5ccc6ccccc6c5c5c4ccc4ccccc45)c3)c3ccccc3-c3ccccc3)cc21 ; CC1(C)c2ccccc2-c2ccc(N(c3ccccc3-c3ccccc3)c3ccccc3-c3ccc4ccc5ccc6ccc7ccccc7c6c5c4c3)cc21 ; CC1(C)c2ccccc2-c2ccc(N(c3ccc(-c4ccc5c(ccc6ccc7ccc8ccccc8c7c65)c4)cc3)c3ccccc3-c3ccccc3)cc21]

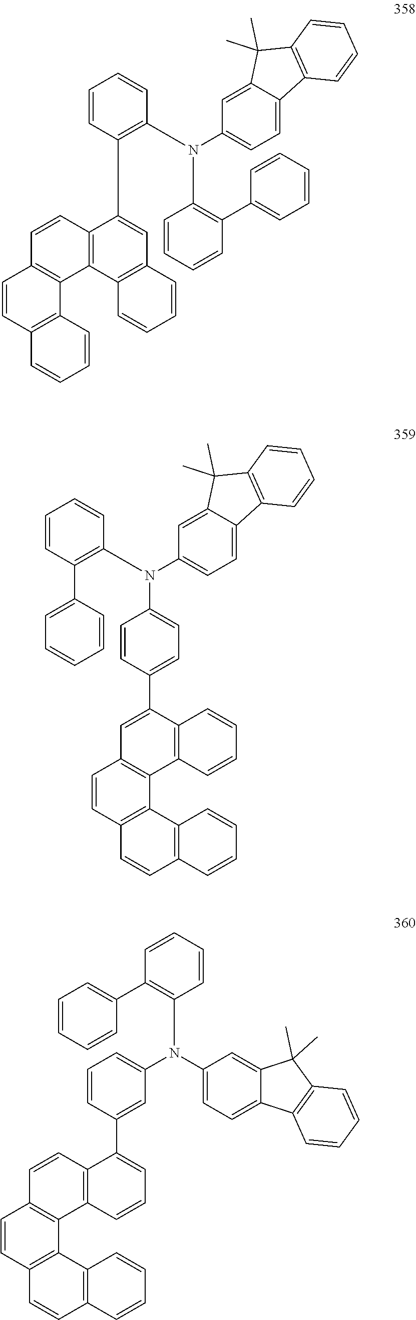 [SMILES: CC1(C)c2ccccc2-c2ccc(N(c3ccccc3-c3ccccc3)c3ccccc3-c3cc4ccccc4c4c3ccc3ccc5ccccc5c34)cc21 ; CC1(C)c2ccccc2-c2ccc(N(c3ccc(-c4cc5ccc6ccc7ccccc7c6c5c5ccccc45)cc3)c3ccccc3-c3ccccc3)cc21 ; CC1(C)c2ccccc2-c2ccc(N(c3cccc(-c4cccc5c4ccc4ccc6ccc7ccccc7c6c45)c3)c3ccccc3-c3ccccc3)cc21]

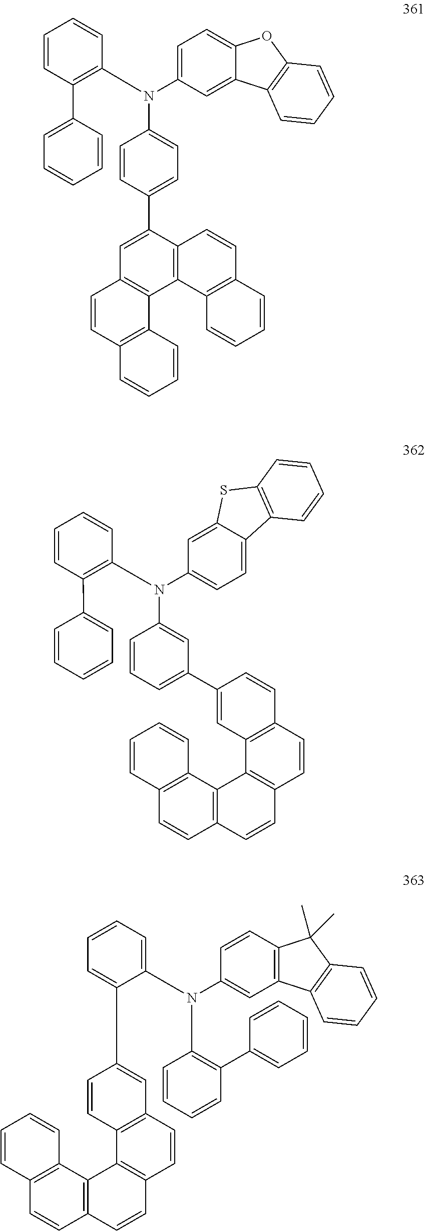 [SMILES: c1ccc(-c2ccccc2N(c2ccc(-c3cc4ccc5ccccc5c4c4c3ccc3ccccc34)cc2)c2ccc3oc4ccccc4c3c2)cc1 ; c1ccc(-c2ccccc2N(c2cccc(-c3ccc4ccc5ccc6ccc7ccccc7c6c5c4c3)c2)c2ccc3c(c2)sc2ccccc23)cc1 ; CC1(C)c2ccccc2-c2cc(N(c3ccccc3-c3ccccc3)c3ccccc3-c3ccc4c(ccc5ccc6ccc7ccccc7c6c54)c3)ccc21]

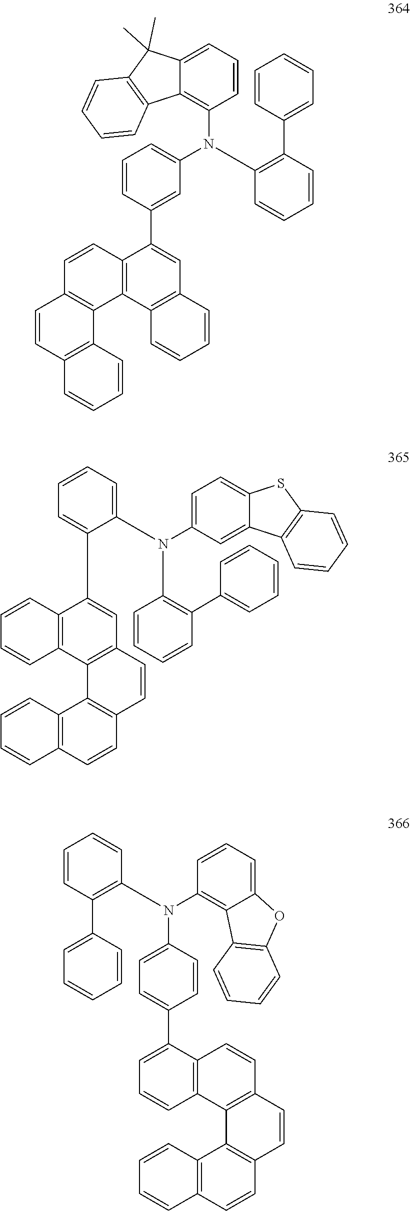 [SMILES: CC1(C)c2ccccc2-c2c(N(c3cccc(-c4cc5ccccc5c5c4ccc4ccc6ccccc6c45)c3)c3ccccc3-c3ccccc3)cccc21 ; c1ccc(-c2ccccc2N(c2ccc3sc4ccccc4c3c2)c2ccccc2-c2cc3ccc4ccc5ccccc5c4c3c3ccccc23)cc1 ; c1ccc(-c2ccccc2N(c2ccc(-c3cccc4c3ccc3ccc5ccc6ccccc6c5c34)cc2)c2cccc3oc4ccccc4c23)cc1]

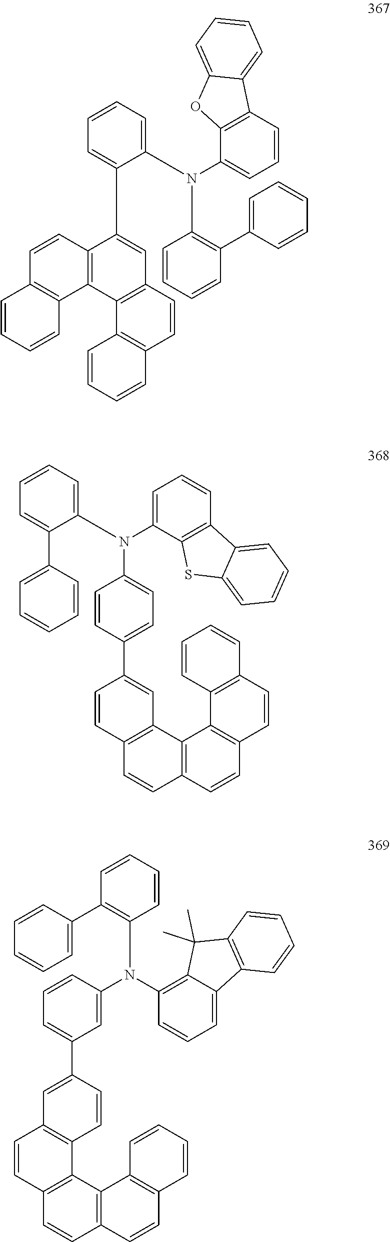 [SMILES: c1ccc(-c2ccccc2N(c2ccccc2-c2cc3ccc4ccccc4c3c3c2ccc2ccccc23)c2cccc3c2oc2ccccc23)cc1 ; c1ccc(-c2ccccc2N(c2ccc(-c3ccc4ccc5ccc6ccc7ccccc7c6c5c4c3)cc2)c2cccc3c2sc2ccccc23)cc1 ; CC1(C)c2ccccc2-c2cccc(N(c3cccc(-c4ccc5c(ccc6ccc7ccc8ccccc8c7c65)c4)c3)c3ccccc3-c3ccccc3)c21]

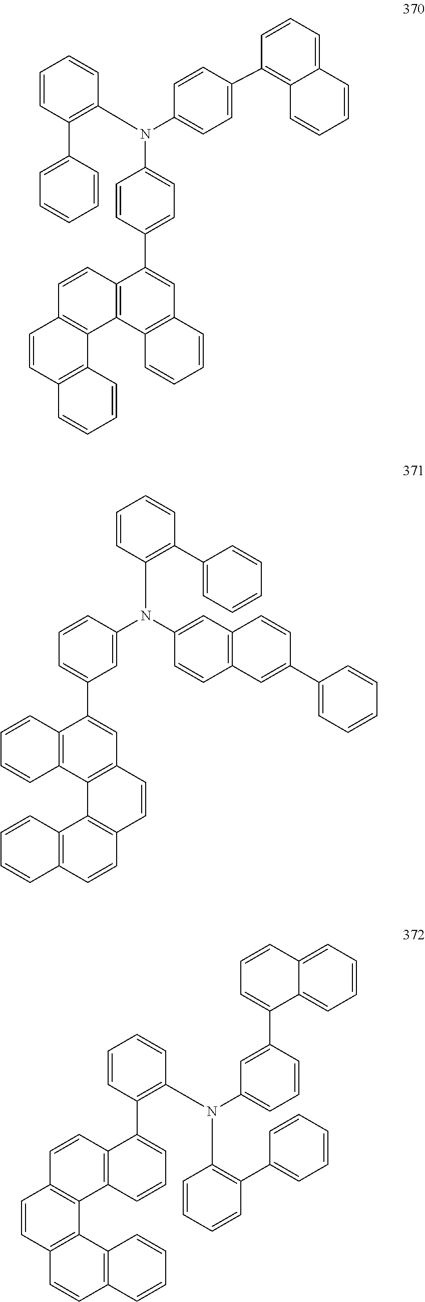 [SMILES: c1ccc(-c2ccccc2N(c2ccc(-c3cccc4ccccc34)cc2)c2ccc(-c3cc4ccccc4c4c3ccc3ccc5ccccc5c34)cc2)cc1 ; c1ccc(-c2ccc3cc(N(c4cccc(-c5cc6ccc7ccc8ccccc8c7c6c6ccccc56)c4)c4ccccc4-c4ccccc4)ccc3c2)cc1 ; c1ccc(-c2ccccc2N(c2cccc(-c3cccc4ccccc34)c2)c2ccccc2-c2cccc3c2ccc2ccc4ccc5ccccc5c4c23)cc1]

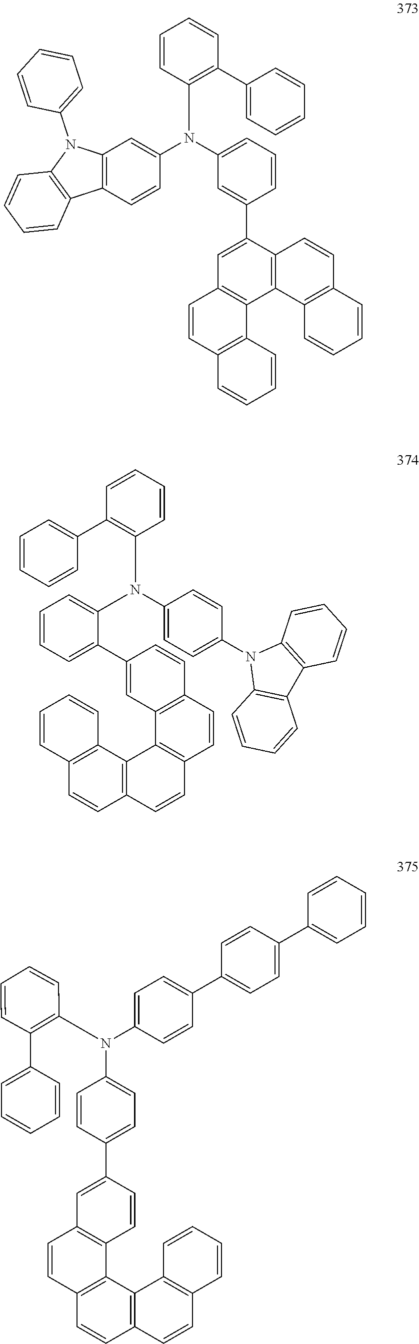 [SMILES: c1ccc(-c2ccccc2N(c2cccc(-c3cc4ccc5ccccc5c4c4c3ccc3ccccc34)c2)c2ccc3c4ccccc4n(-c4ccccc4)c3c2)cc1 ; c1ccc(-c2ccccc2N(c2ccc(-n3c4ccccc4c4ccccc43)cc2)c2ccccc2-c2ccc3ccc4ccc5ccc6ccccc6c5c4c3c2)cc1 ; c1ccc(-c2ccc(-c3ccc(N(c4ccc(-c5ccc6c(ccc7ccc8ccc9ccccc9c8c76)c5)cc4)c4ccccc4-c4ccccc4)cc3)cc2)cc1]

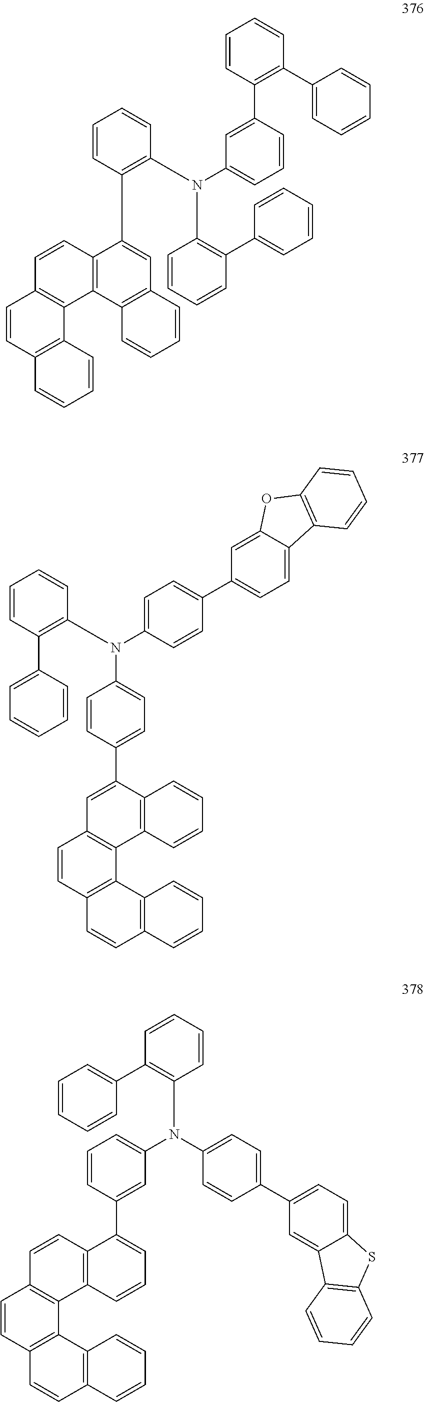 [SMILES: c1ccc(-c2ccccc2-c2cccc(N(c3ccccc3-c3ccccc3)c3ccccc3-c3cc4ccccc4c4c3ccc3ccc5ccccc5c34)c2)cc1 ; c1ccc(-c2ccccc2N(c2ccc(-c3ccc4c(c3)oc3ccccc34)cc2)c2ccc(-c3cc4ccc5ccc6ccccc6c5c4c4ccccc34)cc2)cc1 ; c1ccc(-c2ccccc2N(c2ccc(-c3ccc4sc5ccccc5c4c3)cc2)c2cccc(-c3cccc4c3ccc3ccc5ccc6ccccc6c5c34)c2)cc1]

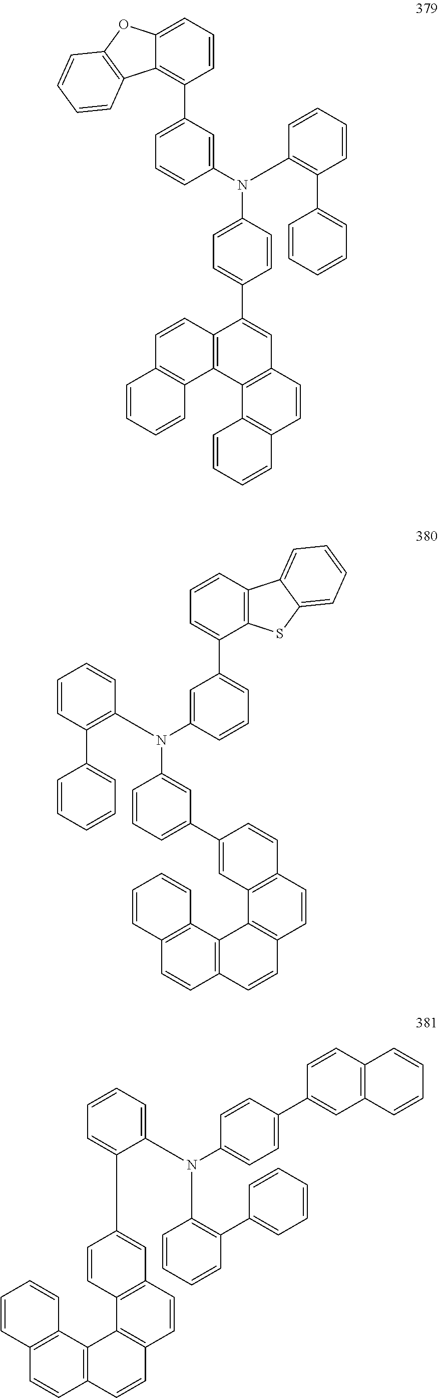 [SMILES: c1ccc(-c2ccccc2N(c2ccc(-c3cc4ccc5ccccc5c4c4c3ccc3ccccc34)cc2)c2cccc(-c3cccc4oc5ccccc5c34)c2)cc1 ; c1ccc(-c2ccccc2N(c2cccc(-c3ccc4ccc5ccc6ccc7ccccc7c6c5c4c3)c2)c2cccc(-c3cccc4c3sc3ccccc34)c2)cc1 ; c1ccc(-c2ccccc2N(c2ccc(-c3ccc4ccccc4c3)cc2)c2ccccc2-c2ccc3c(ccc4ccc5ccc6ccccc6c5c43)c2)cc1]

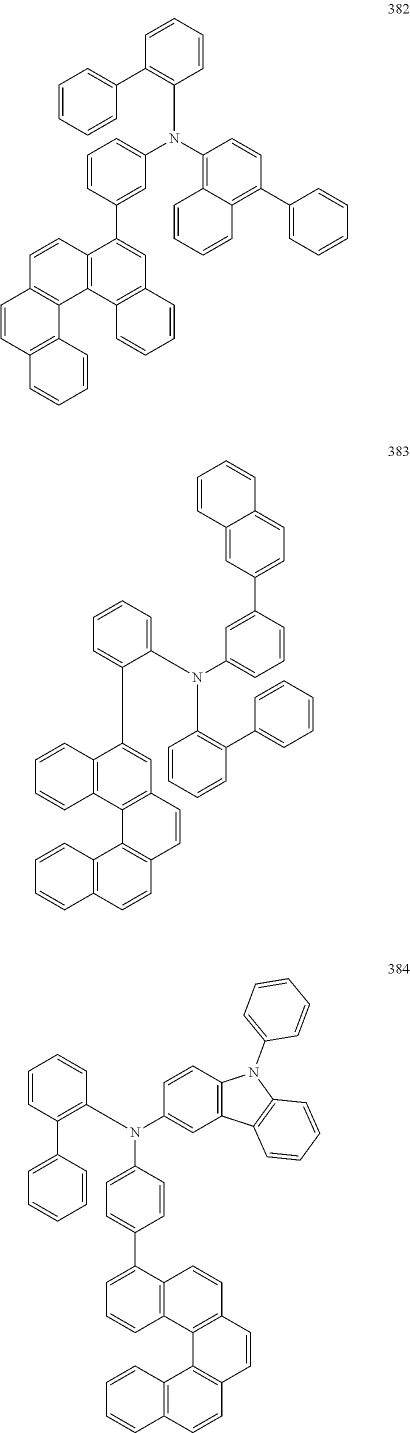 [SMILES: c1ccc(-c2ccccc2N(c2cccc(-c3cc4ccccc4c4c3ccc3ccc5ccccc5c34)c2)c2ccc(-c3ccccc3)c3ccccc23)cc1 ; c1ccc(-c2ccccc2N(c2cccc(-c3ccc4ccccc4c3)c2)c2ccccc2-c2cc3ccc4ccc5ccccc5c4c3c3ccccc23)cc1 ; c1ccc(-c2ccccc2N(c2ccc(-c3cccc4c3ccc3ccc5ccc6ccccc6c5c34)cc2)c2ccc3c(c2)c2ccccc2n3-c2ccccc2)cc1]

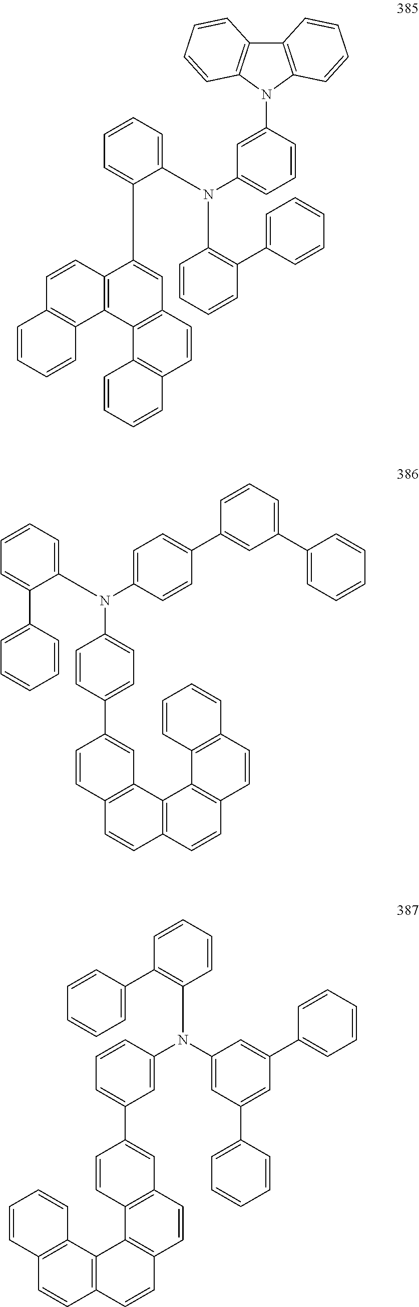 [SMILES: c1ccc(-c2ccccc2N(c2cccc(-n3c4ccccc4c4ccccc43)c2)c2ccccc2-c2cc3ccc4ccccc4c3c3c2ccc2ccccc23)cc1 ; c1ccc(-c2cccc(-c3ccc(N(c4ccc(-c5ccc6ccc7ccc8ccc9ccccc9c8c7c6c5)cc4)c4ccccc4-c4ccccc4)cc3)c2)cc1 ; c1ccc(-c2cc(-c3ccccc3)cc(N(c3cccc(-c4ccc5c(ccc6ccc7ccc8ccccc8c7c65)c4)c3)c3ccccc3-c3ccccc3)c2)cc1]

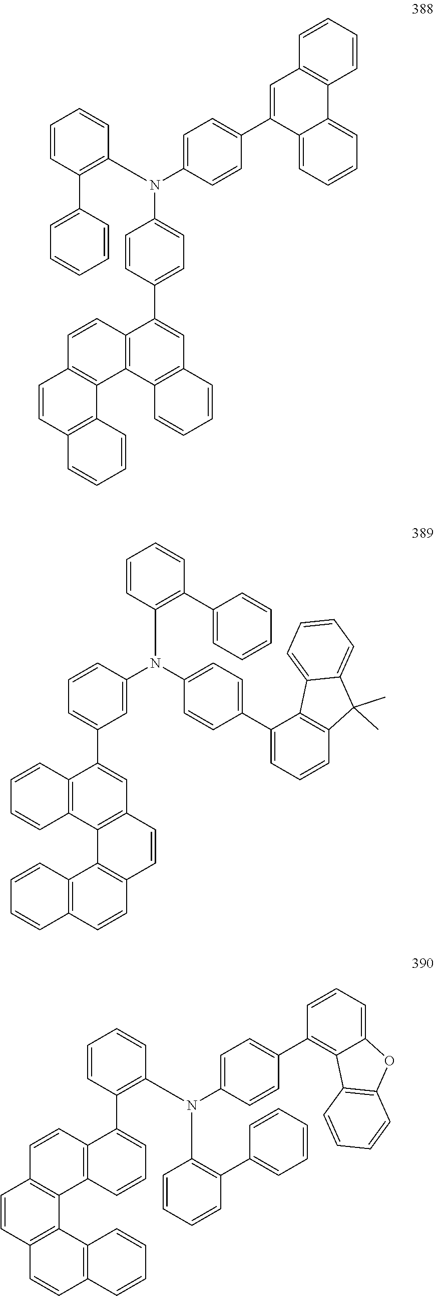 [SMILES: c1ccc(-c2ccccc2N(c2ccc(-c3cc4ccccc4c4ccccc34)cc2)c2ccc(-c3cc4ccccc4c4c3ccc3ccc5ccccc5c34)cc2)cc1 ; CC1(C)c2ccccc2-c2c(-c3ccc(N(c4cccc(-c5cc6ccc7ccc8ccccc8c7c6c6ccccc56)c4)c4ccccc4-c4ccccc4)cc3)cccc21 ; c1ccc(-c2ccccc2N(c2ccc(-c3cccc4oc5ccccc5c34)cc2)c2ccccc2-c2cccc3c2ccc2ccc4ccc5ccccc5c4c23)cc1]

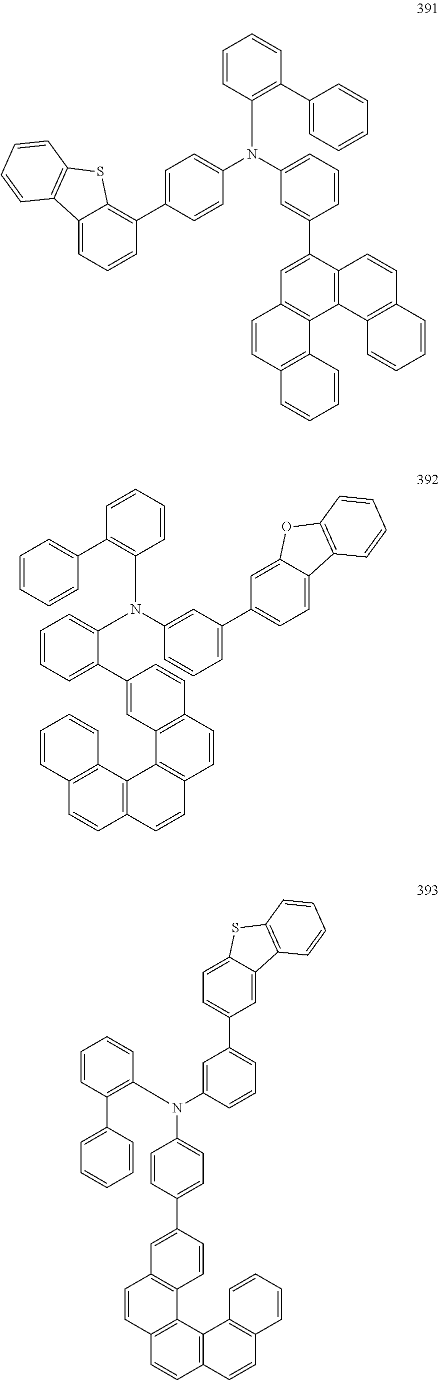 [SMILES: c1ccc(-c2ccccc2N(c2ccc(-c3cccc4c3sc3ccccc34)cc2)c2cccc(-c3cc4ccc5ccccc5c4c4c3ccc3ccccc34)c2)cc1 ; c1ccc(-c2ccccc2N(c2cccc(-c3ccc4c(c3)oc3ccccc34)c2)c2ccccc2-c2ccc3ccc4ccc5ccc6ccccc6c5c4c3c2)cc1 ; c1ccc(-c2ccccc2N(c2ccc(-c3ccc4c(ccc5ccc6ccc7ccccc7c6c54)c3)cc2)c2cccc(-c3ccc4sc5ccccc5c4c3)c2)cc1]

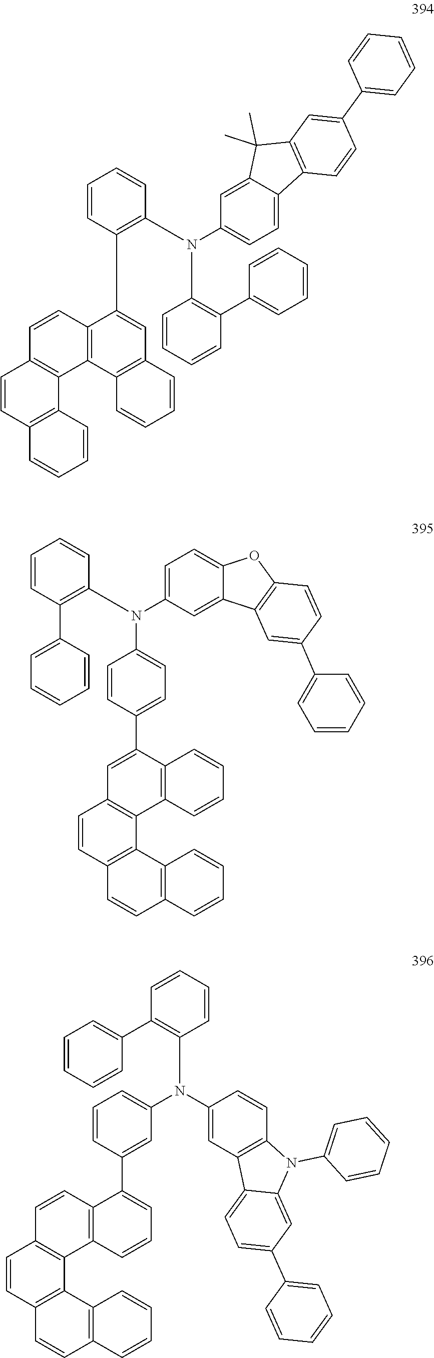 [SMILES: CC1(C)c2cc(-c3ccccc3)ccc2-c2ccc(N(c3ccccc3-c3ccccc3)c3ccccc3-c3cc4ccccc4c4c3ccc3ccc5ccccc5c34)cc21 ; c1ccc(-c2ccc3oc4ccc(N(c5ccc(-c6cc7ccc8ccc9ccccc9c8c7c7ccccc67)cc5)c5ccccc5-c5ccccc5)cc4c3c2)cc1 ; c1ccc(-c2ccc3c4cc(N(c5cccc(-c6cccc7c6ccc6ccc8ccc9ccccc9c8c67)c5)c5ccccc5-c5ccccc5)ccc4n(-c4ccccc4)c3c2)cc1]

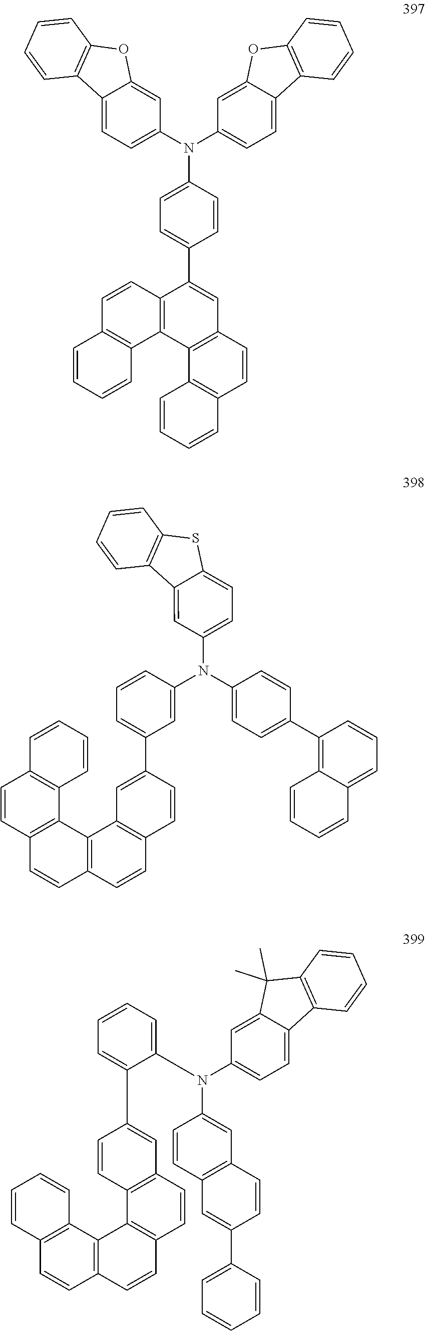 [SMILES: c1ccc2c(c1)ccc1cc(-c3ccc(N(c4ccc5c(c4)oc4ccccc45)c4ccc5c(c4)oc4ccccc45)cc3)c3ccc4ccccc4c3c12 ; c1cc(-c2ccc3ccc4ccc5ccc6ccccc6c5c4c3c2)cc(N(c2ccc(-c3cccc4ccccc34)cc2)c2ccc3sc4ccccc4c3c2)c1 ; CC1(C)c2ccccc2-c2ccc(N(c3ccc4cc(-c5ccccc5)ccc4c3)c3ccccc3-c3ccc4c(ccc5ccc6ccc7ccccc7c6c54)c3)cc21]

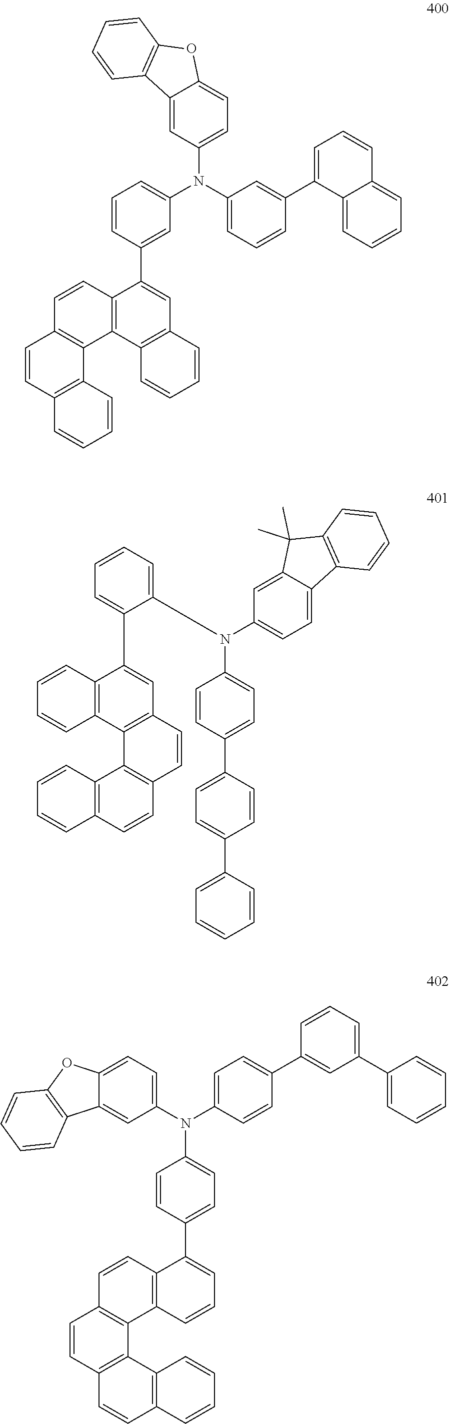 [SMILES: c1cc(-c2cccc3ccccc23)cc(N(c2cccc(-c3cc4ccccc4c4c3ccc3ccc5ccccc5c34)c2)c2ccc3oc4ccccc4c3c2)c1 ; CC1(C)c2ccccc2-c2ccc(N(c3ccc(-c4ccc(-c5ccccc5)cc4)cc3)c3ccccc3-c3cc4ccc5ccc6ccccc6c5c4c4ccccc34)cc21 ; c1ccc(-c2cccc(-c3ccc(N(c4ccc(-c5cccc6c5ccc5ccc7ccc8ccccc8c7c56)cc4)c4ccc5oc6ccccc6c5c4)cc3)c2)cc1]

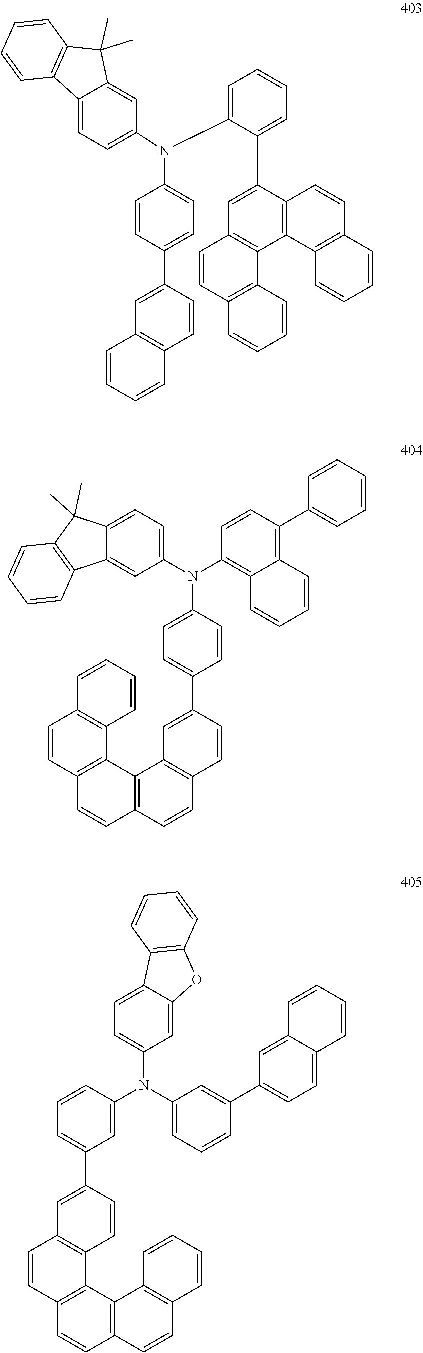 [SMILES: CC1(C)c2ccccc2-c2ccc(N(c3ccc(-c4ccc5ccccc5c4)cc3)c3ccccc3-c3cc4ccc5ccccc5c4c4c3ccc3ccccc34)cc21 ; CC1(C)c2ccccc2-c2cc(N(c3ccc(-c4ccc5ccc6ccc7ccc8ccccc8c7c6c5c4)cc3)c3ccc(-c4ccccc4)c4ccccc34)ccc21 ; c1cc(-c2ccc3ccccc3c2)cc(N(c2cccc(-c3ccc4c(ccc5ccc6ccc7ccccc7c6c54)c3)c2)c2ccc3c(c2)oc2ccccc23)c1]

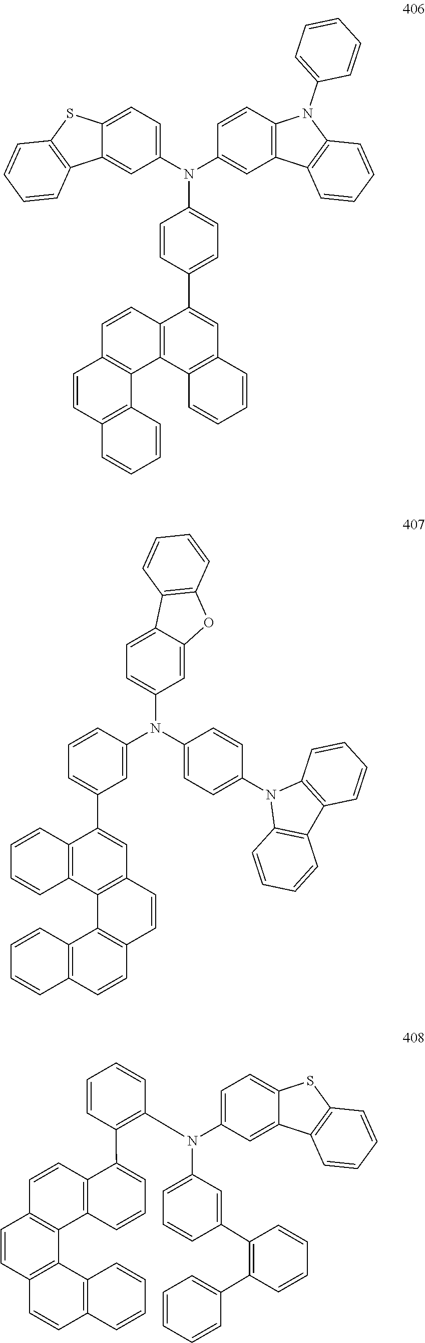 [SMILES: c1ccc(-n2c3ccccc3c3cc(N(c4ccc(-c5cc6ccccc6c6c5ccc5ccc7ccccc7c56)cc4)c4ccc5sc6ccccc6c5c4)ccc32)cc1 ; c1cc(-c2cc3ccc4ccc5ccccc5c4c3c3ccccc23)cc(N(c2ccc(-n3c4ccccc4c4ccccc43)cc2)c2ccc3c(c2)oc2ccccc23)c1 ; c1ccc(-c2ccccc2-c2cccc(N(c3ccc4sc5ccccc5c4c3)c3ccccc3-c3cccc4c3ccc3ccc5ccc6ccccc6c5c34)c2)cc1]

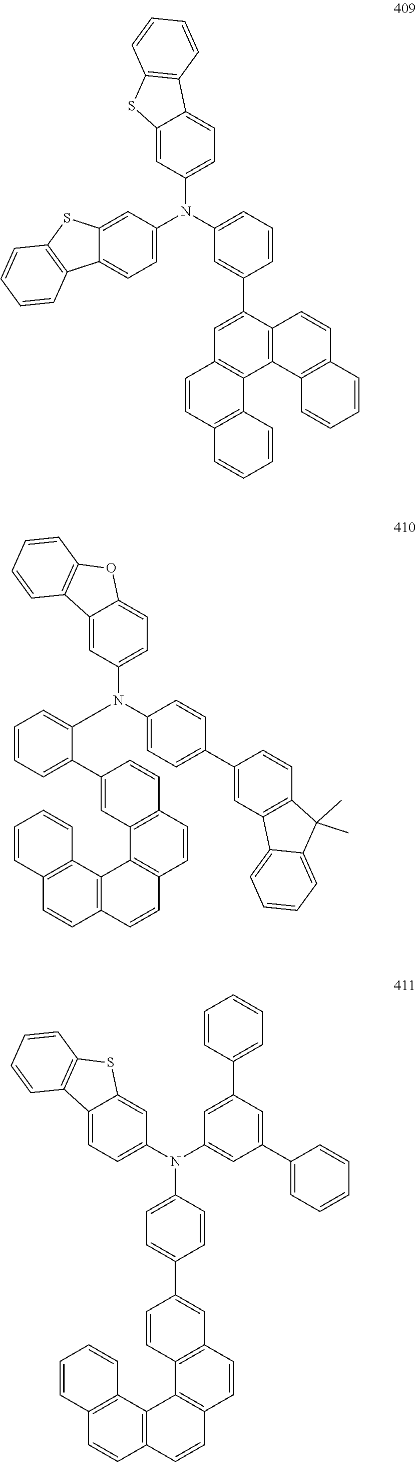 [SMILES: c1cc(-c2cc3ccc4ccccc4c3c3c2ccc2ccccc23)cc(N(c2ccc3c(c2)sc2ccccc23)c2ccc3c(c2)sc2ccccc23)c1 ; CC1(C)c2ccccc2-c2cc(-c3ccc(N(c4ccc5oc6ccccc6c5c4)c4ccccc4-c4ccc5ccc6ccc7ccc8ccccc8c7c6c5c4)cc3)ccc21 ; c1ccc(-c2cc(-c3ccccc3)cc(N(c3ccc(-c4ccc5c(ccc6ccc7ccc8ccccc8c7c65)c4)cc3)c3ccc4c(c3)sc3ccccc34)c2)cc1]

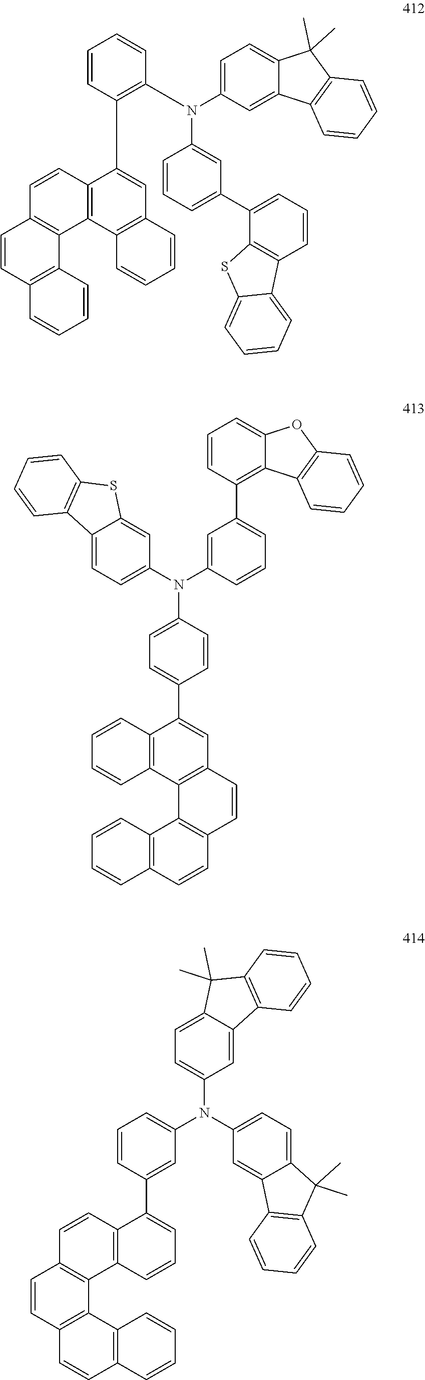 [SMILES: CC1(C)c2ccccc2-c2cc(N(c3cccc(-c4cccc5c4sc4ccccc45)c3)c3ccccc3-c3cc4ccccc4c4c3ccc3ccc5ccccc5c34)ccc21 ; c1cc(-c2cccc3oc4ccccc4c23)cc(N(c2ccc(-c3cc4ccc5ccc6ccccc6c5c4c4ccccc34)cc2)c2ccc3c(c2)sc2ccccc23)c1 ; CC1(C)c2ccccc2-c2cc(N(c3cccc(-c4cccc5c4ccc4ccc6ccc7ccccc7c6c45)c3)c3ccc4c(c3)-c3ccccc3C4(C)C)ccc21]

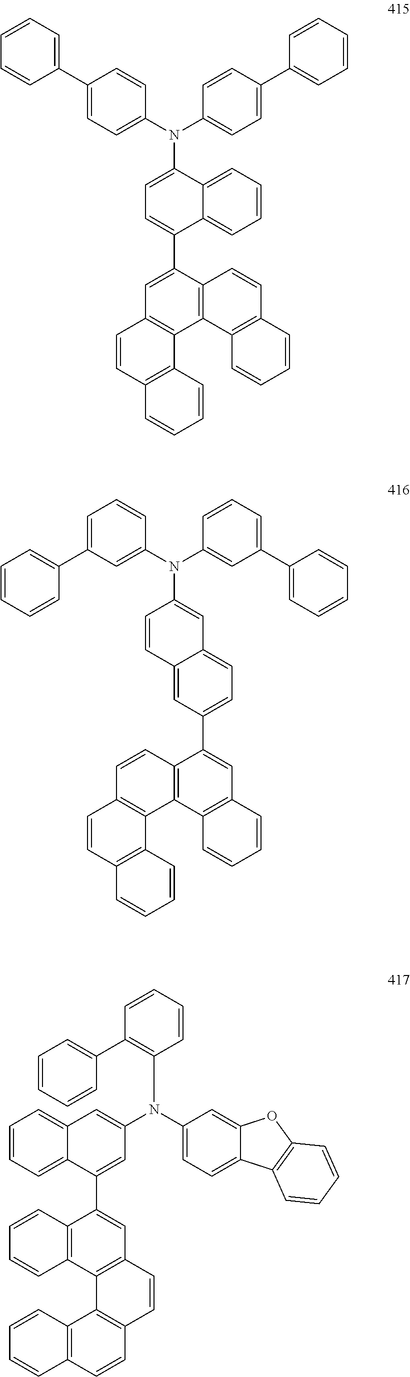 [SMILES: c1ccc(-c2ccc(N(c3ccc(-c4ccccc4)cc3)c3ccc(-c4cc5ccc6ccccc6c5c5c4ccc4ccccc45)c4ccccc34)cc2)cc1 ; c1ccc(-c2cccc(N(c3cccc(-c4ccccc4)c3)c3ccc4cc(-c5cc6ccccc6c6c5ccc5ccc7ccccc7c56)ccc4c3)c2)cc1 ; c1ccc(-c2ccccc2N(c2cc(-c3cc4ccc5ccc6ccccc6c5c4c4ccccc34)c3ccccc3c2)c2ccc3c(c2)oc2ccccc23)cc1]

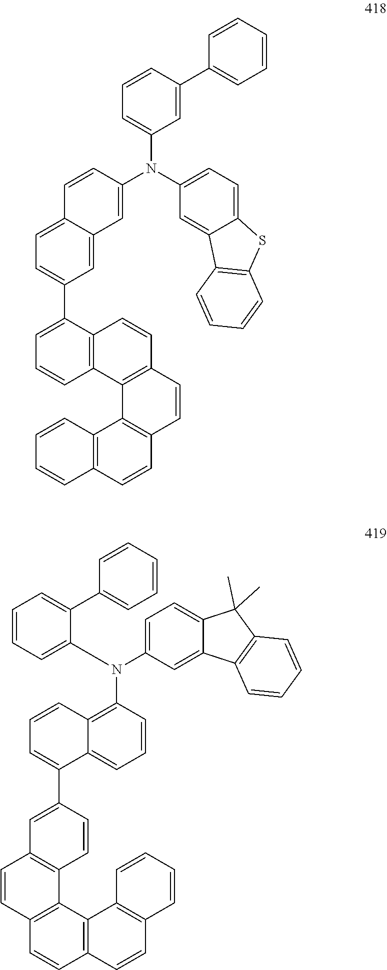 [SMILES: c1ccc(-c2cccc(N(c3ccc4ccc(-c5cccc6c5ccc5ccc7ccc8ccccc8c7c56)cc4c3)c3ccc4sc5ccccc5c4c3)c2)cc1 ; CC1(C)c2ccccc2-c2cc(N(c3ccccc3-c3ccccc3)c3cccc4c(-c5ccc6c(ccc7ccc8ccc9ccccc9c8c76)c5)cccc34)ccc21]

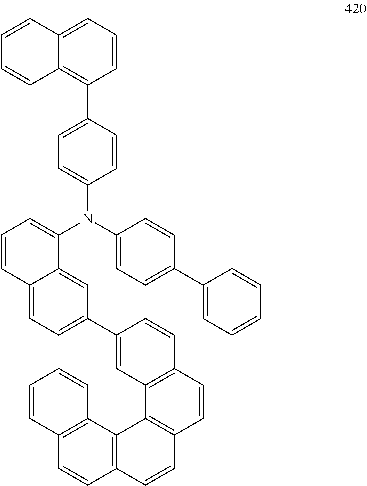 [SMILES: c1ccc(-c2ccc(N(c3ccc(-c4cccc5ccccc45)cc3)c3cccc4ccc(-c5ccc6ccc7ccc8ccc9ccccc9c8c7c6c5)cc34)cc2)cc1]

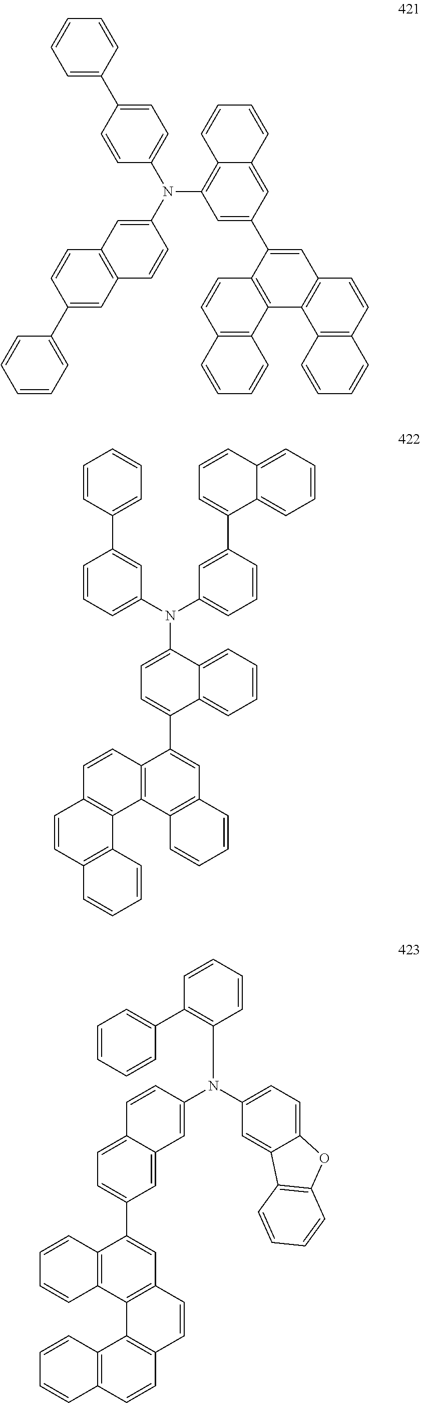 [SMILES: c1ccc(-c2ccc(N(c3ccc4cc(-c5ccccc5)ccc4c3)c3cc(-c4cc5ccc6ccccc6c5c5c4ccc4ccccc45)cc4ccccc34)cc2)cc1 ; c1ccc(-c2cccc(N(c3cccc(-c4cccc5ccccc45)c3)c3ccc(-c4cc5ccccc5c5c4ccc4ccc6ccccc6c45)c4ccccc34)c2)cc1 ; c1ccc(-c2ccccc2N(c2ccc3ccc(-c4cc5ccc6ccc7ccccc7c6c5c5ccccc45)cc3c2)c2ccc3oc4ccccc4c3c2)cc1]

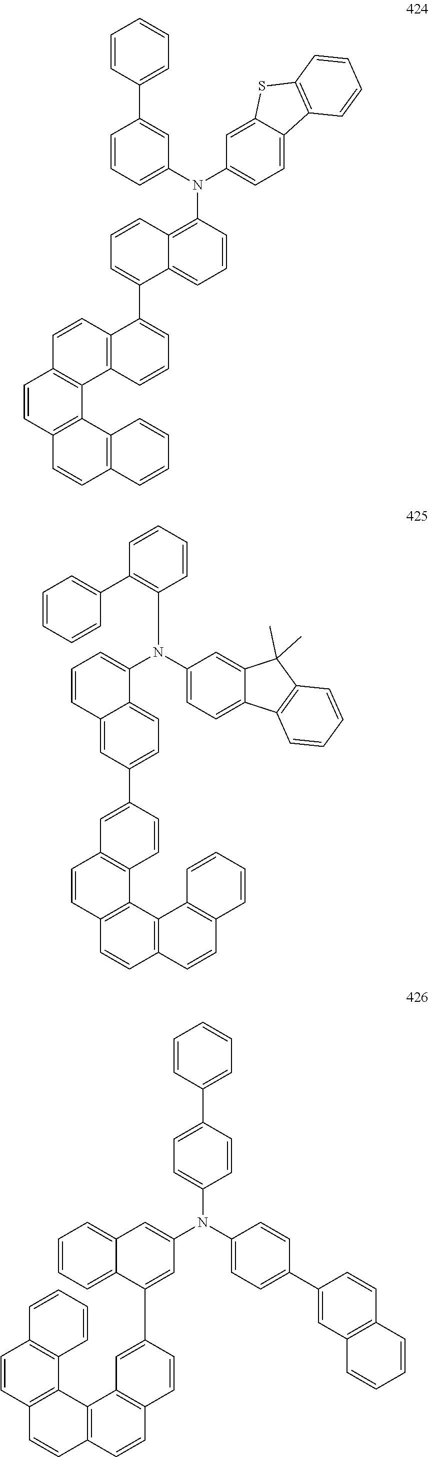

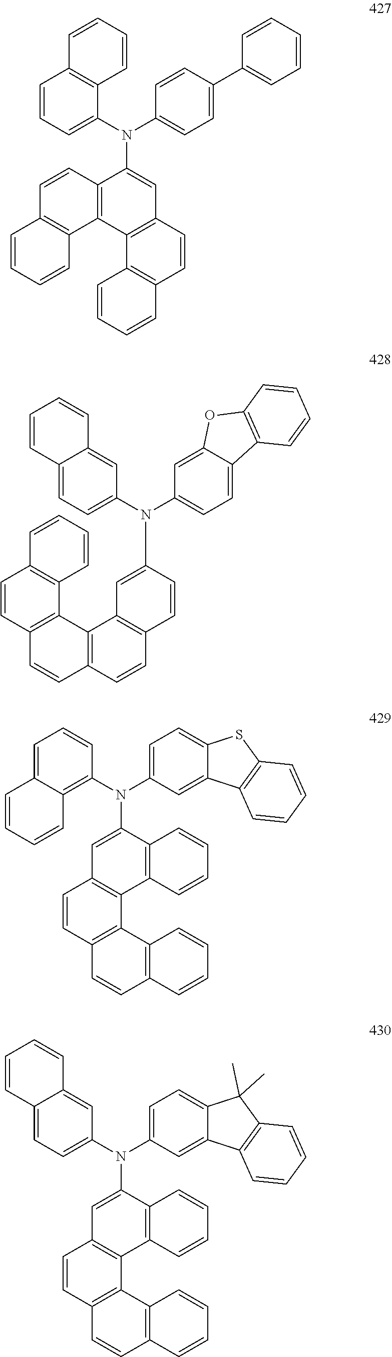

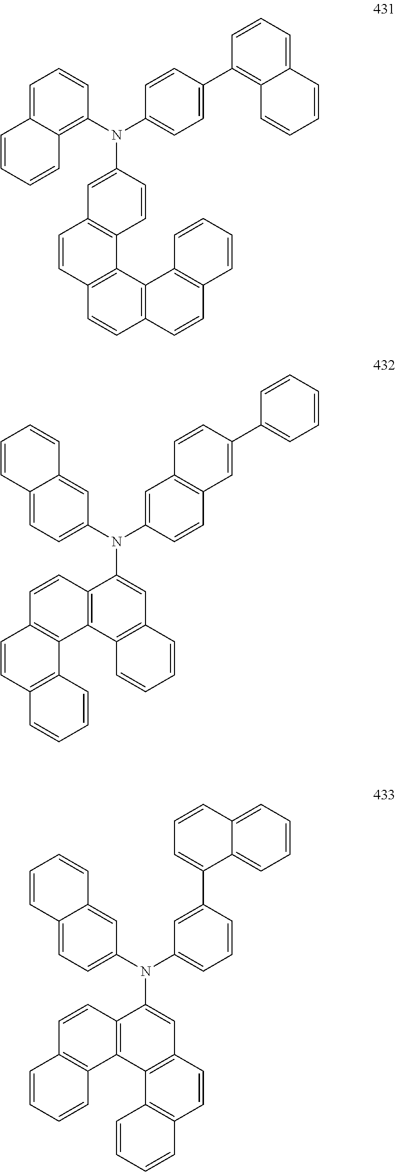 [SMILES: c1ccc2c(-c3ccc(N(c4ccc5c(ccc6ccc7ccc8ccccc8c7c65)c4)c4cccc5ccccc45)cc3)cccc2c1 ; c1ccc(-c2ccc3cc(N(c4ccc5ccccc5c4)c4cc5ccccc5c5c4ccc4ccc6ccccc6c45)ccc3c2)cc1 ; c1cc(-c2cccc3ccccc23)cc(N(c2ccc3ccccc3c2)c2cc3ccc4ccccc4c3c3c2ccc2ccccc23)c1]

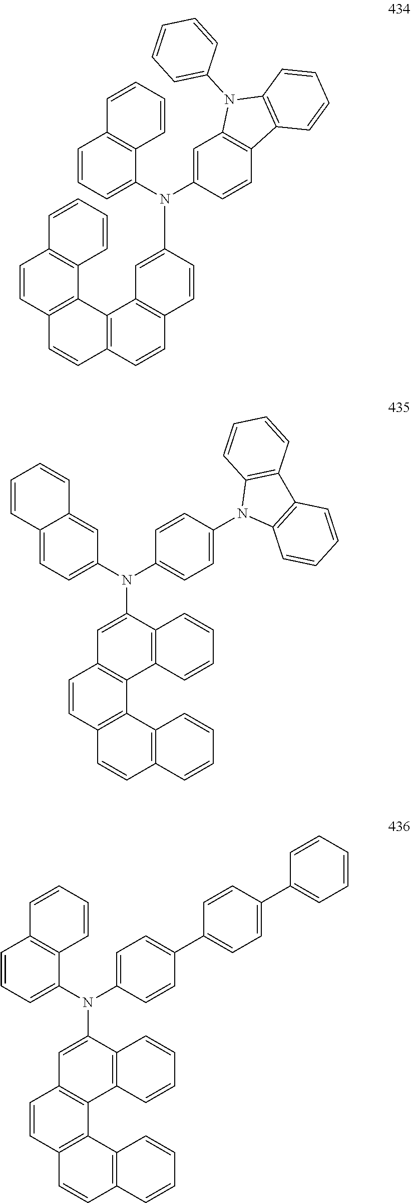 [SMILES: c1ccc(-n2c3ccccc3c3ccc(N(c4ccc5ccc6ccc7ccc8ccccc8c7c6c5c4)c4cccc5ccccc45)cc32)cc1 ; c1ccc2cc(N(c3ccc(-n4c5ccccc5c5ccccc54)cc3)c3cc4ccc5ccc6ccccc6c5c4c4ccccc34)ccc2c1 ; c1ccc(-c2ccc(-c3ccc(N(c4cccc5ccccc45)c4cc5ccc6ccc7ccccc7c6c5c5ccccc45)cc3)cc2)cc1]

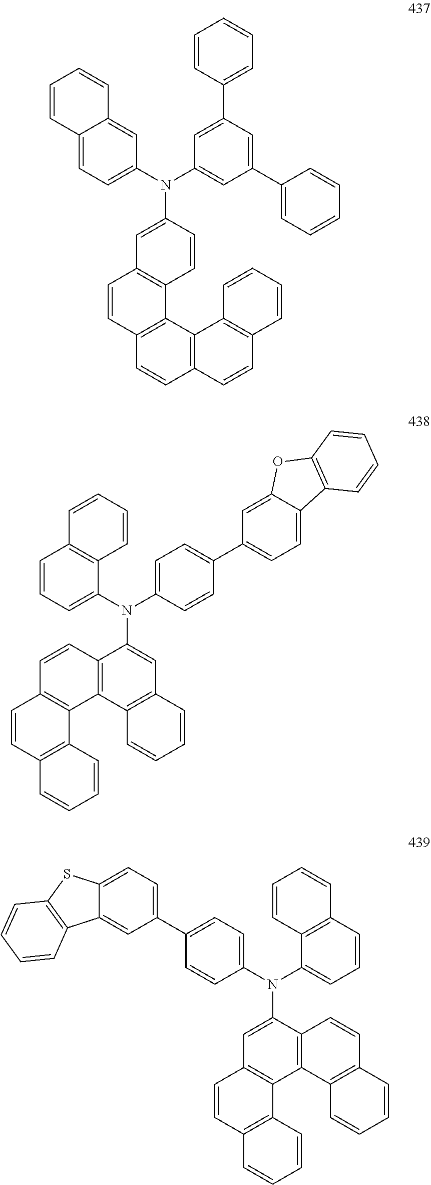 [SMILES: c1ccc(-c2cc(-c3ccccc3)cc(N(c3ccc4ccccc4c3)c3ccc4c(ccc5ccc6ccc7ccccc7c6c54)c3)c2)cc1 ; c1ccc2c(N(c3ccc(-c4ccc5c(c4)oc4ccccc45)cc3)c3cc4ccccc4c4c3ccc3ccc5ccccc5c34)cccc2c1 ; c1ccc2c(N(c3ccc(-c4ccc5sc6ccccc6c5c4)cc3)c3cc4ccc5ccccc5c4c4c3ccc3ccccc34)cccc2c1]

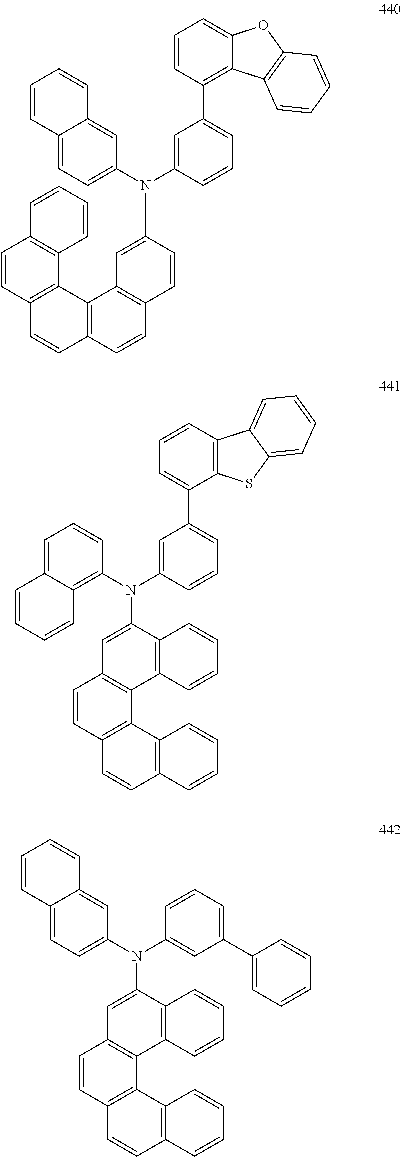 [SMILES: c1cc(-c2cccc3oc4ccccc4c23)cc(N(c2ccc3ccccc3c2)c2ccc3ccc4ccc5ccc6ccccc6c5c4c3c2)c1 ; c1cc(-c2cccc3c2sc2ccccc23)cc(N(c2cccc3ccccc23)c2cc3ccc4ccc5ccccc5c4c3c3ccccc23)c1 ; c1ccc(-c2cccc(N(c3ccc4ccccc4c3)c3cc4ccc5ccc6ccccc6c5c4c4ccccc34)c2)cc1]

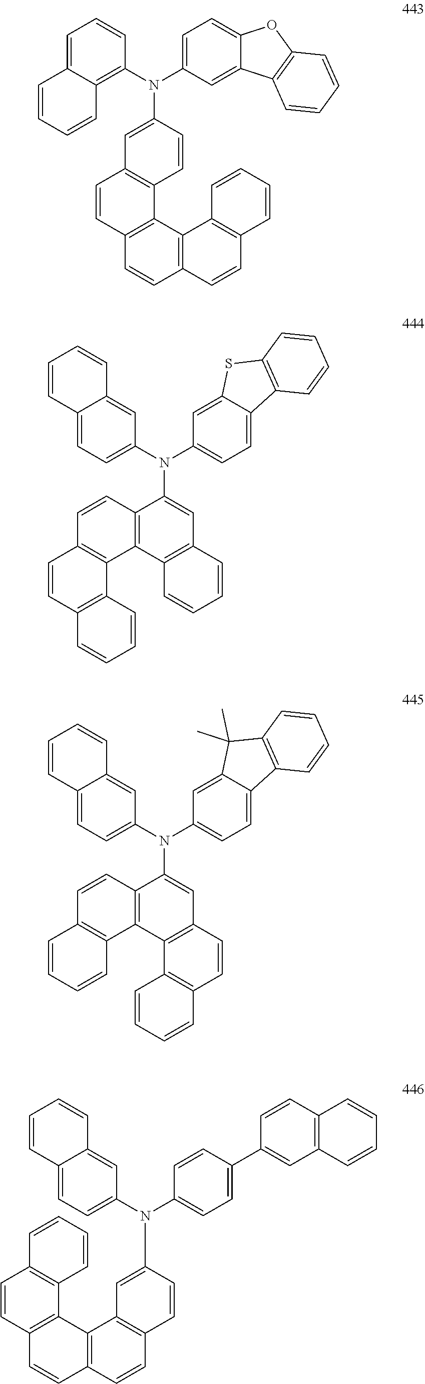 [SMILES: c1ccc2c(N(c3ccc4c(ccc5ccc6ccc7ccccc7c6c54)c3)c3ccc4oc5ccccc5c4c3)cccc2c1 ; c1ccc2cc(N(c3ccc4c(c3)sc3ccccc34)c3cc4ccccc4c4c3ccc3ccc5ccccc5c34)ccc2c1 ; CC1(C)c2ccccc2-c2ccc(N(c3ccc4ccccc4c3)c3cc4ccc5ccccc5c4c4c3ccc3ccccc34)cc21 ; c1ccc2cc(-c3ccc(N(c4ccc5ccccc5c4)c4ccc5ccc6ccc7ccc8ccccc8c7c6c5c4)cc3)ccc2c1]

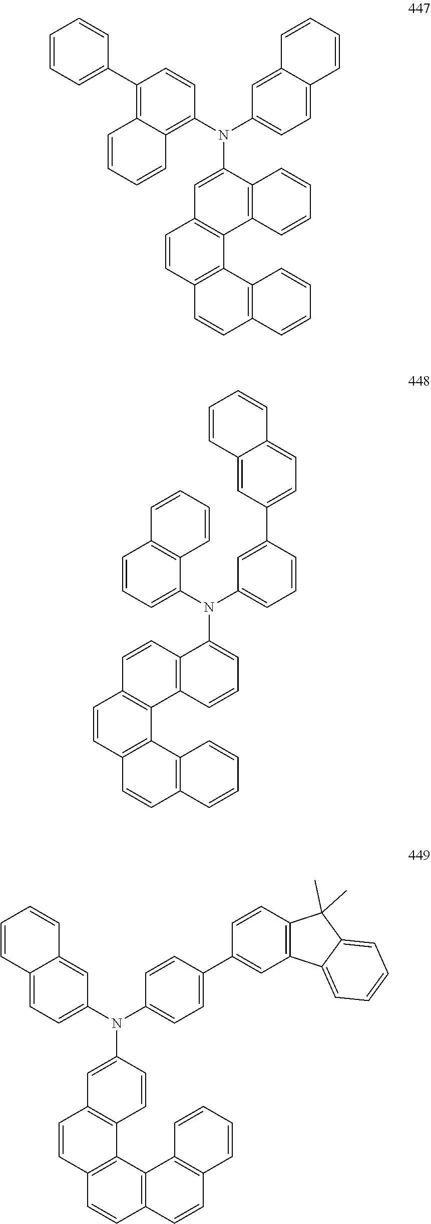 [SMILES: c1ccc(-c2ccc(N(c3ccc4ccccc4c3)c3cc4ccc5ccc6ccccc6c5c4c4ccccc34)c3ccccc23)cc1 ; c1cc(-c2ccc3ccccc3c2)cc(N(c2cccc3ccccc23)c2cccc3c2ccc2ccc4ccc5ccccc5c4c23)c1 ; CC1(C)c2ccccc2-c2cc(-c3ccc(N(c4ccc5ccccc5c4)c4ccc5c(ccc6ccc7ccc8ccccc8c7c65)c4)cc3)ccc21]

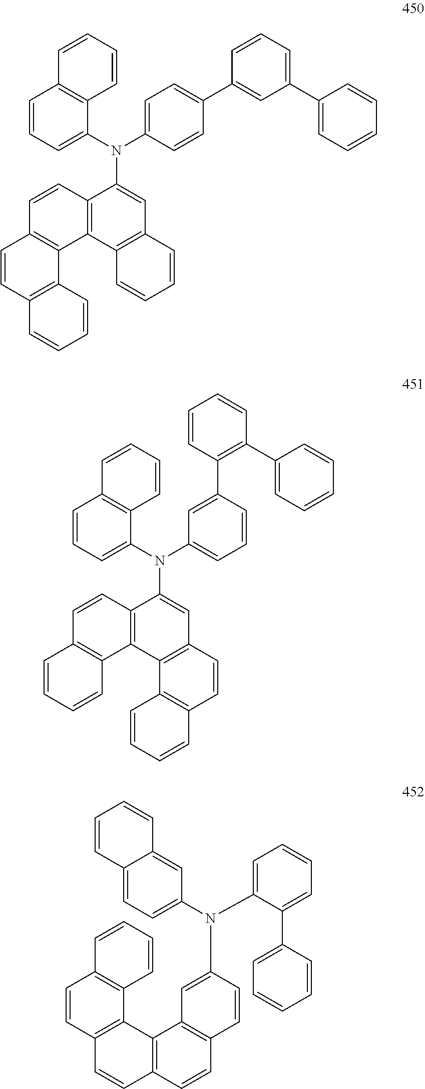 [SMILES: c1ccc(-c2cccc(-c3ccc(N(c4cccc5ccccc45)c4cc5ccccc5c5c4ccc4ccc6ccccc6c45)cc3)c2)cc1 ; c1ccc(-c2ccccc2-c2cccc(N(c3cccc4ccccc34)c3cc4ccc5ccccc5c4c4c3ccc3ccccc34)c2)cc1 ; c1ccc(-c2ccccc2N(c2ccc3ccccc3c2)c2ccc3ccc4ccc5ccc6ccccc6c5c4c3c2)cc1]

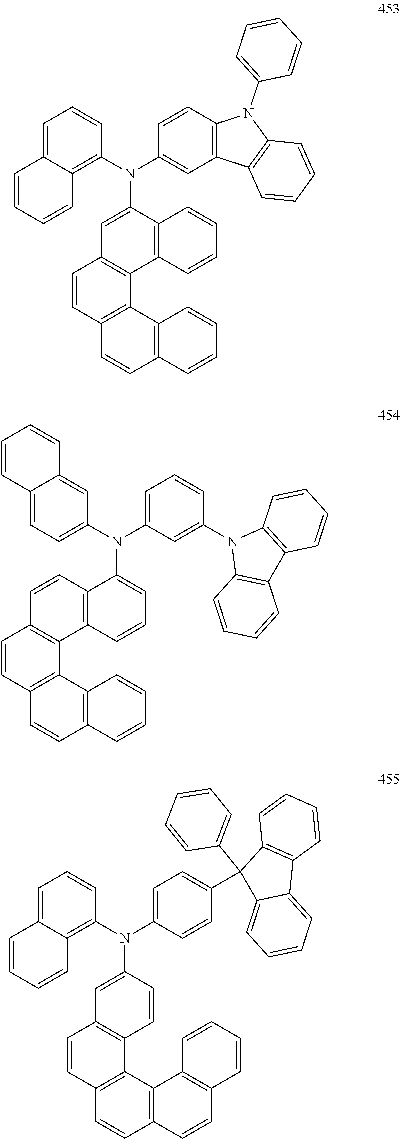 [SMILES: c1ccc(-n2c3ccccc3c3cc(N(c4cccc5ccccc45)c4cc5ccc6ccc7ccccc7c6c5c5ccccc45)ccc32)cc1 ; c1cc(N(c2ccc3ccccc3c2)c2cccc3c2ccc2ccc4ccc5ccccc5c4c23)cc(-n2c3ccccc3c3ccccc32)c1 ; c1ccc(C2(c3ccc(N(c4ccc5c(ccc6ccc7ccc8ccccc8c7c65)c4)c4cccc5ccccc45)cc3)c3ccccc3-c3ccccc32)cc1]

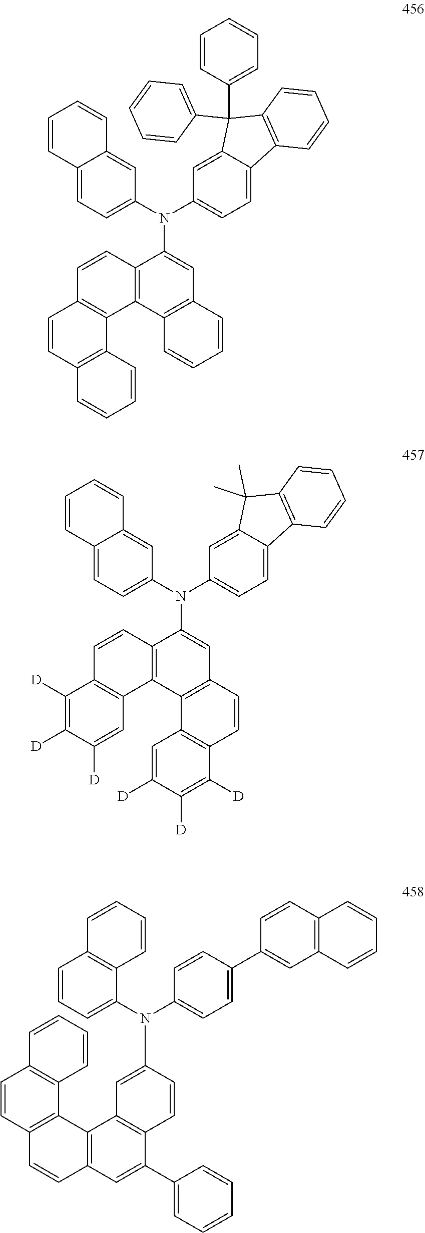 [SMILES: c1ccc(C2(c3ccccc3)c3ccccc3-c3ccc(N(c4ccc5ccccc5c4)c4cc5ccccc5c5c4ccc4ccc6ccccc6c45)cc32)cc1 ; [2H]c1cc2c(ccc3cc(N(c4ccc5c(c4)C(C)(C)c4ccccc4-5)c4ccc5ccccc5c4)c4ccc5c([2H])c([2H])c([2H])cc5c4c32)c([2H])c1[2H] ; c1ccc(-c2cc3ccc4ccc5ccccc5c4c3c3cc(N(c4ccc(-c5ccc6ccccc6c5)cc4)c4cccc5ccccc45)ccc23)cc1]

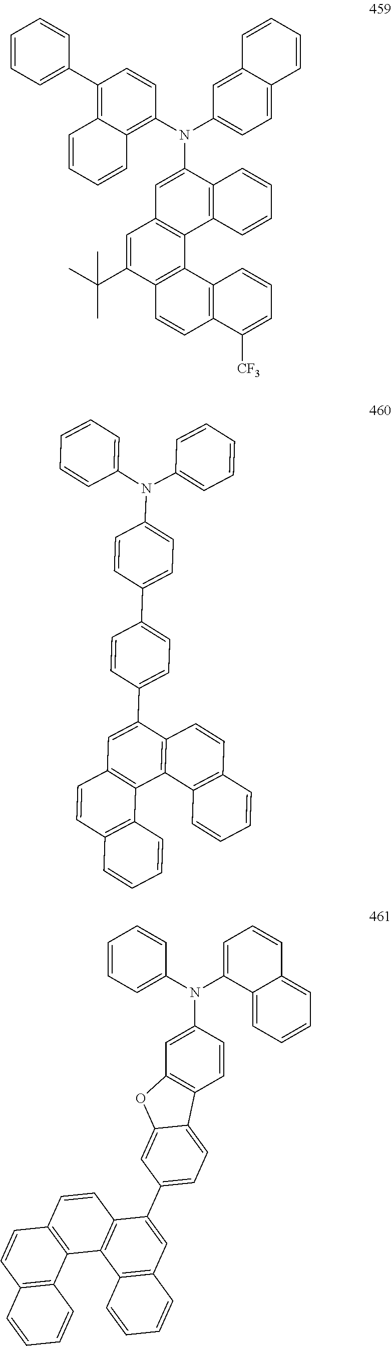

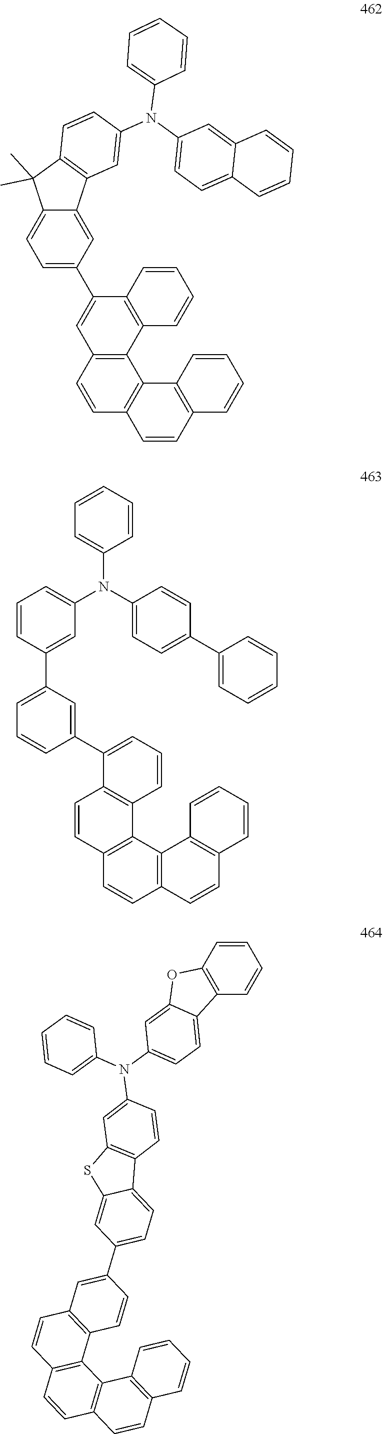 [SMILES: CC1(C)c2ccc(-c3cc4ccc5ccc6ccccc6c5c4c4ccccc34)cc2-c2cc(N(c3ccccc3)c3ccc4ccccc4c3)ccc21 ; c1ccc(-c2ccc(N(c3ccccc3)c3cccc(-c4cccc(-c5cccc6c5ccc5ccc7ccc8ccccc8c7c56)c4)c3)cc2)cc1 ; c1ccc(N(c2ccc3c(c2)oc2ccccc23)c2ccc3c(c2)sc2cc(-c4ccc5c(ccc6ccc7ccc8ccccc8c7c65)c4)ccc23)cc1]

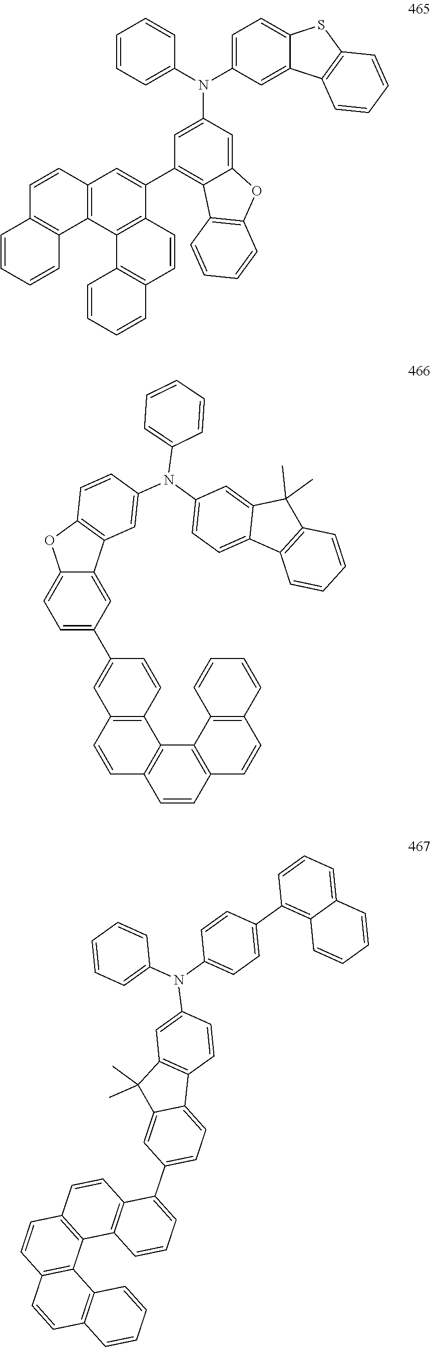 [SMILES: c1ccc(N(c2cc(-c3cc4ccc5ccccc5c4c4c3ccc3ccccc34)c3c(c2)oc2ccccc23)c2ccc3sc4ccccc4c3c2)cc1 ; CC1(C)c2ccccc2-c2ccc(N(c3ccccc3)c3ccc4oc5ccc(-c6ccc7c(ccc8ccc9ccc%10ccccc%10c9c87)c6)cc5c4c3)cc21 ; CC1(C)c2cc(-c3cccc4c3ccc3ccc5ccc6ccccc6c5c34)ccc2-c2ccc(N(c3ccccc3)c3ccc(-c4cccc5ccccc45)cc3)cc21]

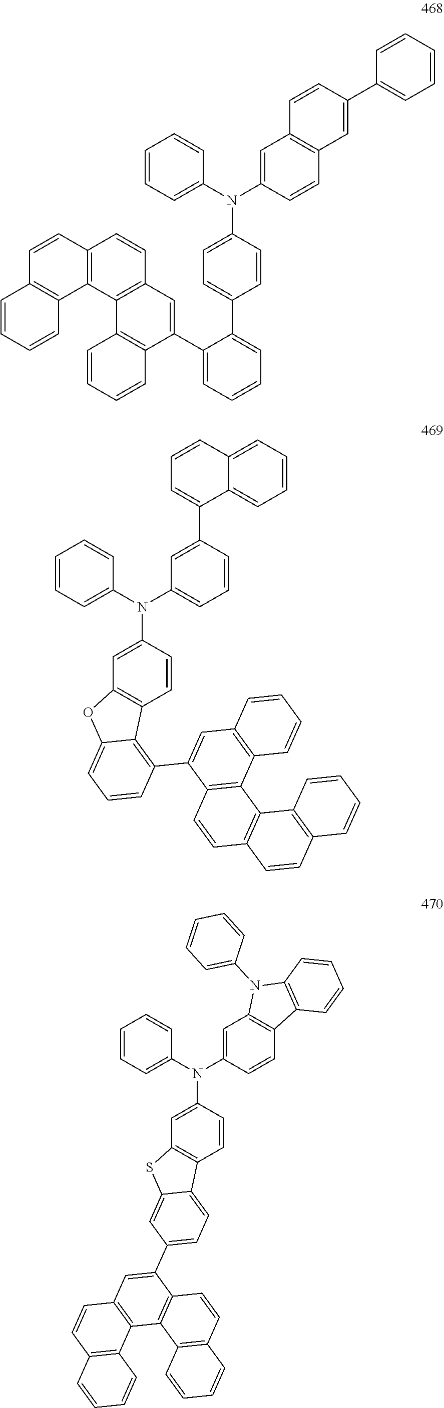 [SMILES: c1ccc(-c2ccc3cc(N(c4ccccc4)c4ccc(-c5ccccc5-c5cc6ccc7ccc8ccccc8c7c6c6ccccc56)cc4)ccc3c2)cc1 ; c1ccc(N(c2cccc(-c3cccc4ccccc34)c2)c2ccc3c(c2)oc2cccc(-c4cc5ccccc5c5c4ccc4ccc6ccccc6c45)c23)cc1 ; c1ccc(N(c2ccc3c(c2)sc2cc(-c4cc5ccc6ccccc6c5c5c4ccc4ccccc45)ccc23)c2ccc3c4ccccc4n(-c4ccccc4)c3c2)cc1]

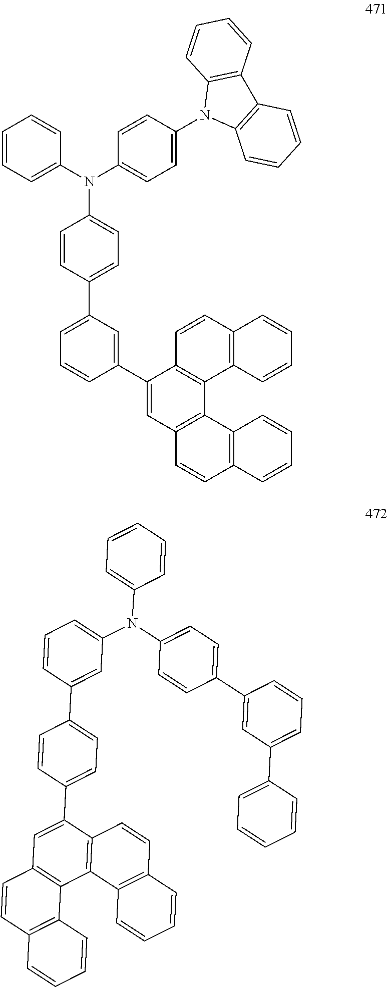 [SMILES: c1ccc(N(c2ccc(-c3cccc(-c4cc5ccc6ccccc6c5c5c4ccc4ccccc45)c3)cc2)c2ccc(-n3c4ccccc4c4ccccc43)cc2)cc1 ; c1ccc(-c2cccc(-c3ccc(N(c4ccccc4)c4cccc(-c5ccc(-c6cc7ccc8ccccc8c7c7c6ccc6ccccc67)cc5)c4)cc3)c2)cc1]

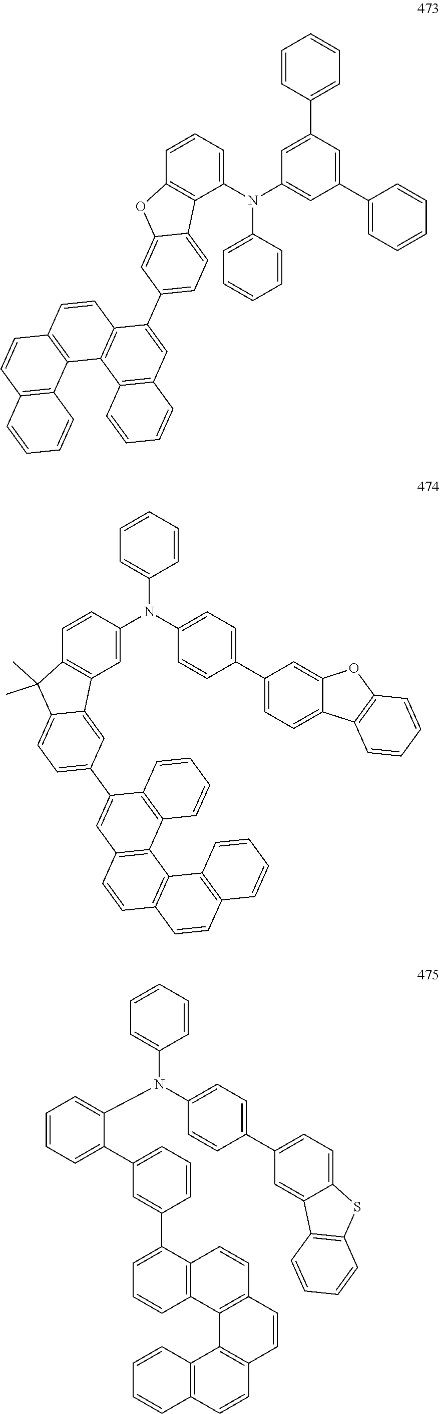 [SMILES: c1ccc(-c2cc(-c3ccccc3)cc(N(c3ccccc3)c3cccc4oc5cc(-c6cc7ccccc7c7c6ccc6ccc8ccccc8c67)ccc5c34)c2)cc1 ; CC1(C)c2ccc(-c3cc4ccc5ccc6ccccc6c5c4c4ccccc34)cc2-c2cc(N(c3ccccc3)c3ccc(-c4ccc5c(c4)oc4ccccc45)cc3)ccc21 ; c1ccc(N(c2ccc(-c3ccc4sc5ccccc5c4c3)cc2)c2ccccc2-c2cccc(-c3cccc4c3ccc3ccc5ccc6ccccc6c5c34)c2)cc1]

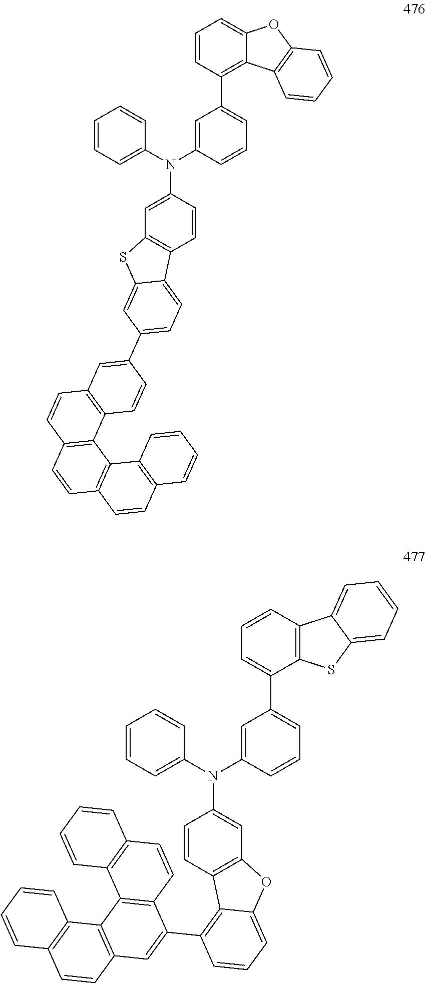 [SMILES: c1ccc(N(c2cccc(-c3cccc4oc5ccccc5c34)c2)c2ccc3c(c2)sc2cc(-c4ccc5c(ccc6ccc7ccc8ccccc8c7c65)c4)ccc23)cc1 ; c1ccc(N(c2cccc(-c3cccc4c3sc3ccccc34)c2)c2ccc3c(c2)oc2cccc(-c4cc5ccc6ccccc6c5c5c4ccc4ccccc45)c23)cc1]

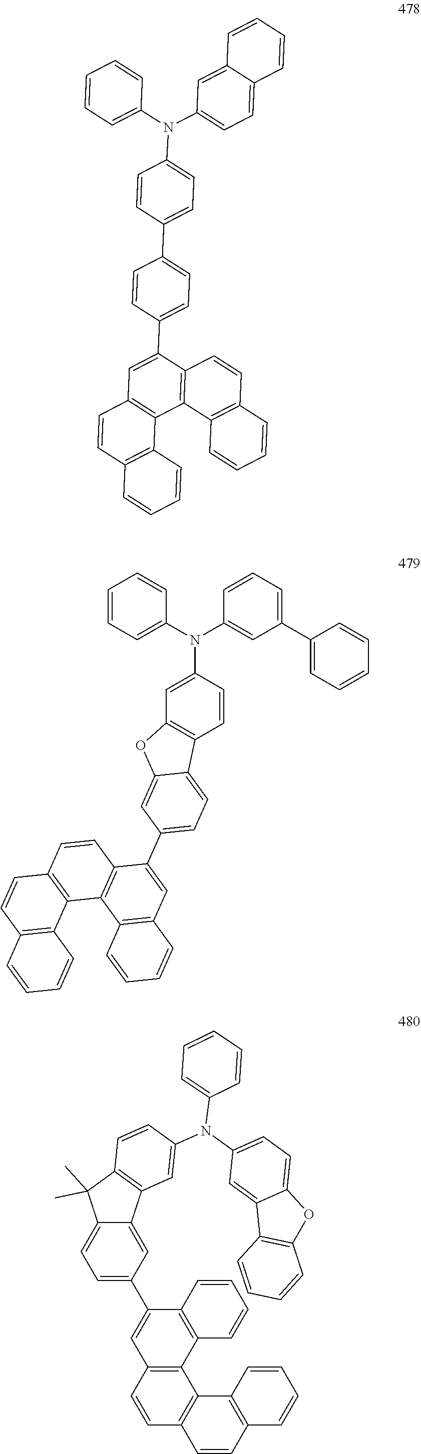 [SMILES: c1ccc(N(c2ccc(-c3ccc(-c4cc5ccc6ccccc6c5c5c4ccc4ccccc45)cc3)cc2)c2ccc3ccccc3c2)cc1 ; c1ccc(-c2cccc(N(c3ccccc3)c3ccc4c(c3)oc3cc(-c5cc6ccccc6c6c5ccc5ccc7ccccc7c56)ccc34)c2)cc1 ; CC1(C)c2ccc(-c3cc4ccc5ccc6ccccc6c5c4c4ccccc34)cc2-c2cc(N(c3ccccc3)c3ccc4oc5ccccc5c4c3)ccc21]

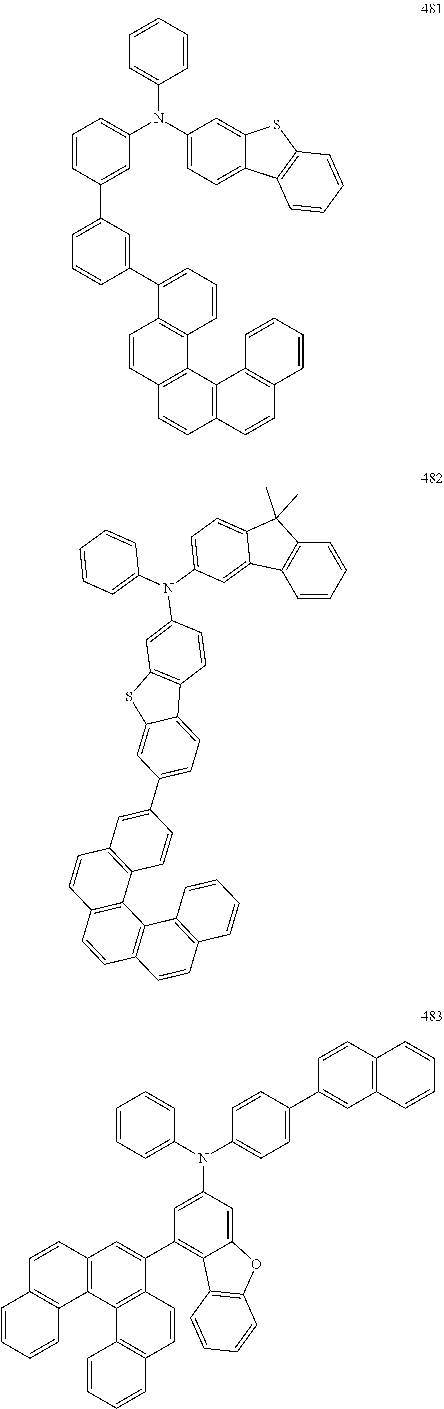 [SMILES: c1ccc(N(c2cccc(-c3cccc(-c4cccc5c4ccc4ccc6ccc7ccccc7c6c45)c3)c2)c2ccc3c(c2)sc2ccccc23)cc1 ; CC1(C)c2ccccc2-c2cc(N(c3ccccc3)c3ccc4c(c3)sc3cc(-c5ccc6c(ccc7ccc8ccc9ccccc9c8c76)c5)ccc34)ccc21 ; c1ccc(N(c2ccc(-c3ccc4ccccc4c3)cc2)c2cc(-c3cc4ccc5ccccc5c4c4c3ccc3ccccc34)c3c(c2)oc2ccccc23)cc1]

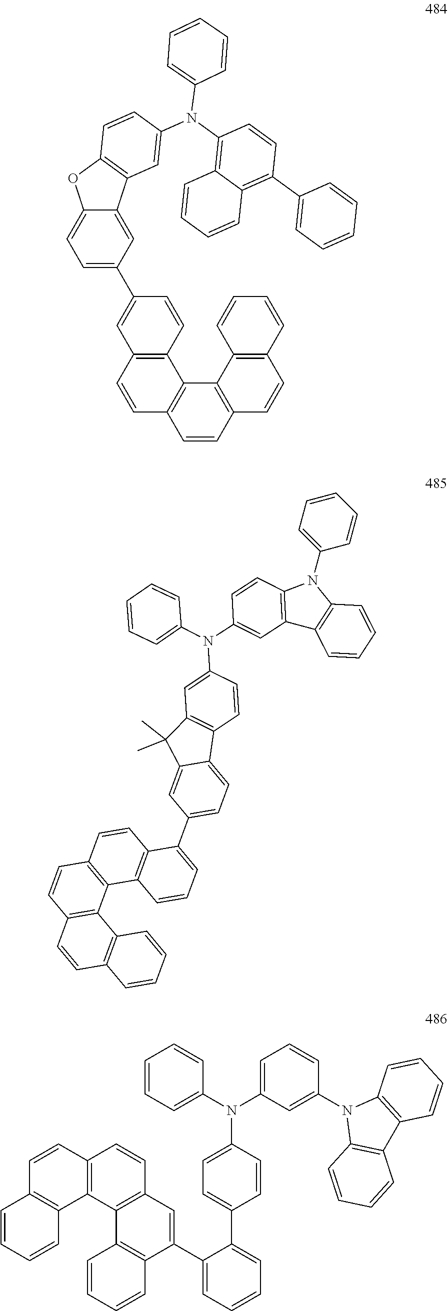 [SMILES: c1ccc(-c2ccc(N(c3ccccc3)c3ccc4oc5ccc(-c6ccc7c(ccc8ccc9ccc%10ccccc%10c9c87)c6)cc5c4c3)c3ccccc23)cc1 ; CC1(C)c2cc(-c3cccc4c3ccc3ccc5ccc6ccccc6c5c34)ccc2-c2ccc(N(c3ccccc3)c3ccc4c(c3)c3ccccc3n4-c3ccccc3)cc21 ; c1ccc(N(c2ccc(-c3ccccc3-c3cc4ccc5ccc6ccccc6c5c4c4ccccc34)cc2)c2cccc(-n3c4ccccc4c4ccccc43)c2)cc1]

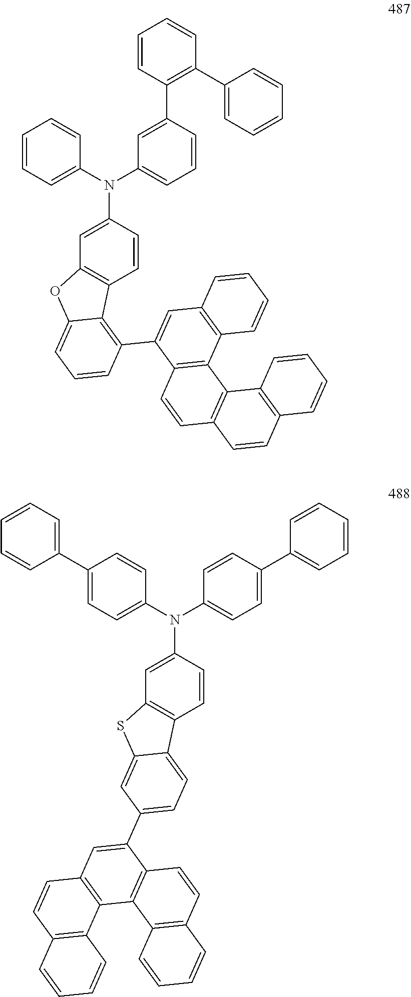 [SMILES: c1ccc(-c2ccccc2-c2cccc(N(c3ccccc3)c3ccc4c(c3)oc3cccc(-c5cc6ccccc6c6c5ccc5ccc7ccccc7c56)c34)c2)cc1 ; c1ccc(-c2ccc(N(c3ccc(-c4ccccc4)cc3)c3ccc4c(c3)sc3cc(-c5cc6ccc7ccccc7c6c6c5ccc5ccccc56)ccc34)cc2)cc1]

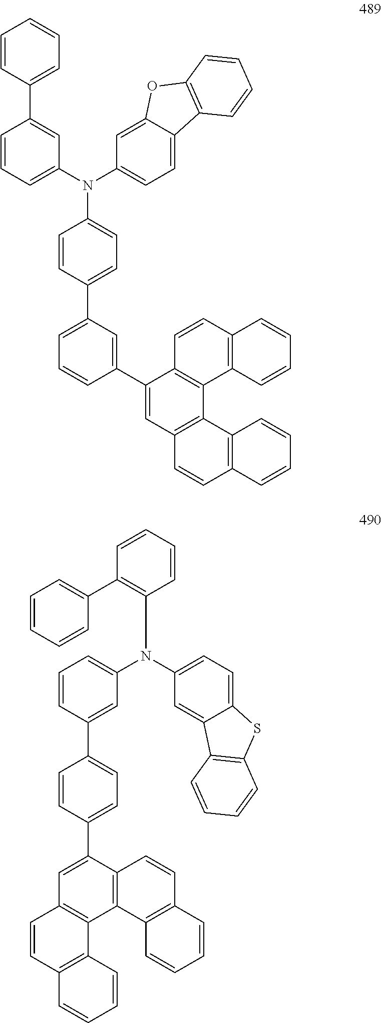 [SMILES: c1ccc(-c2cccc(N(c3ccc(-c4cccc(-c5cc6ccc7ccccc7c6c6c5ccc5ccccc56)c4)cc3)c3ccc4c(c3)oc3ccccc34)c2)cc1 ; c1ccc(-c2ccccc2N(c2cccc(-c3ccc(-c4cc5ccc6ccccc6c5c5c4ccc4ccccc45)cc3)c2)c2ccc3sc4ccccc4c3c2)cc1]

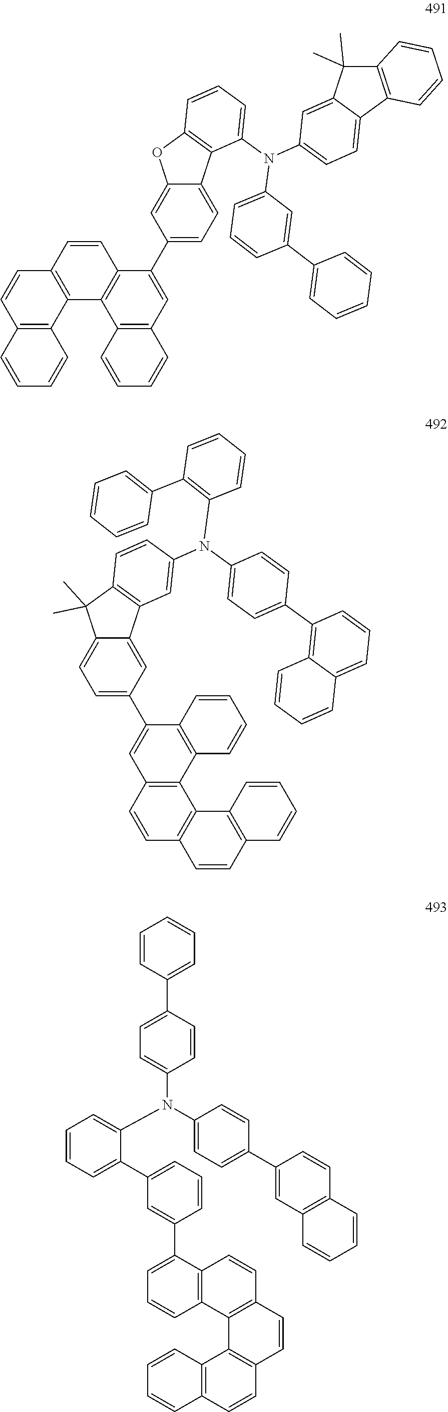 [SMILES: CC1(C)c2ccccc2-c2ccc(N(c3cccc(-c4ccccc4)c3)c3cccc4oc5cc(-c6cc7ccccc7c7c6ccc6ccc8ccccc8c67)ccc5c34)cc21 ; CC1(C)c2ccc(-c3cc4ccc5ccc6ccccc6c5c4c4ccccc34)cc2-c2cc(N(c3ccc(-c4cccc5ccccc45)cc3)c3ccccc3-c3ccccc3)ccc21 ; c1ccc(-c2ccc(N(c3ccc(-c4ccc5ccccc5c4)cc3)c3ccccc3-c3cccc(-c4cccc5c4ccc4ccc6ccc7ccccc7c6c45)c3)cc2)cc1]

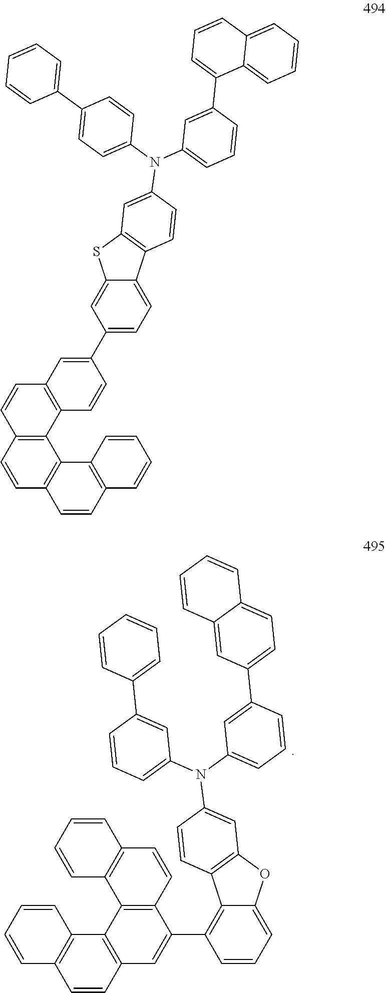 [SMILES: c1ccc(-c2ccc(N(c3cccc(-c4cccc5ccccc45)c3)c3ccc4c(c3)sc3cc(-c5ccc6c(ccc7ccc8ccc9ccccc9c8c76)c5)ccc34)cc2)cc1 ; c1ccc(-c2cccc(N(c3cccc(-c4ccc5ccccc5c4)c3)c3ccc4c(c3)oc3cccc(-c5cc6ccc7ccccc7c6c6c5ccc5ccccc56)c34)c2)cc1]

In a second aspect, the disclosure provides an organic electroluminescent device, including an anode, a cathode, and a functional layer disposed between the anode and the cathode; where the functional layer includes the arylamine compound according to the first aspect of the disclosure.

The arylamine compound provided in the disclosure can be used to form at least one organic film layer in the functional layer so as to improve the characteristics such as the luminous efficiency and service life of the organic electroluminescent device.

Optionally, the functional layer includes an organic electroluminescent layer including the arylamine compound. The organic electroluminescent layer may be composed of the arylamine compound provided by the disclosure, or may be composed of the arylamine compound provided by the disclosure and other materials together.

Optionally, the functional layer further includes a hole transport layer (also referred to as a first hole transport layer) and a hole adjustment layer (also referred to as a second hole transport layer), the hole transport layer being located between the anode and the organic electroluminescent layer, and the hole adjustment layer being located between the hole transport layer and the organic electroluminescent layer. In some embodiments, the hole adjustment layer is composed of the arylamine compound provided by the disclosure, or is composed of the arylamine compound provided by the disclosure and other materials together.

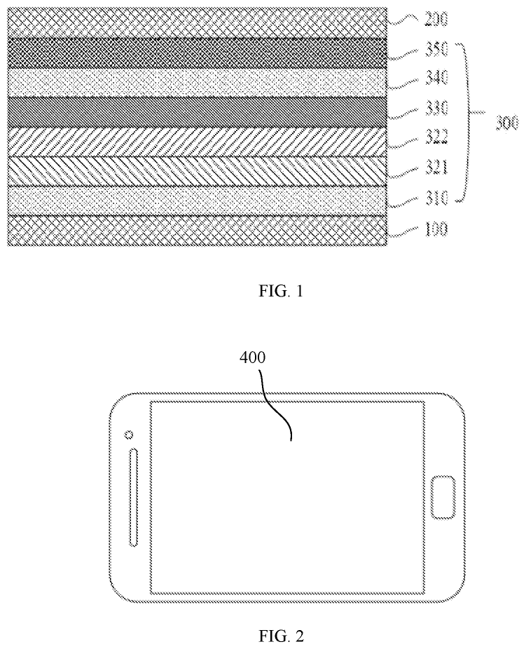

According to one specific embodiment, the organic electroluminescent device is shown in FIG. 1, and includes an anode 100, a hole injection layer 310, a hole transport layer 321, a hole adjustment layer 322, an organic electroluminescent layer 330, an electron transport layer 340, an electron injection layer 350 and a cathode 200 which are sequentially stacked.

In the disclosure, the anode 100 includes an anode material, which is preferably a material having a large work function that facilitates hole injection into the functional layer. Specific examples of the anode material include metals such as nickel, platinum, vanadium, chromium, copper, zinc, and gold, or an alloy of them; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy) thiophene] (PEDT), polypyrrole, and polyaniline, but are not limited to this. A transparent electrode containing indium tin oxide (ITO) as the anode is preferably included.

In the disclosure, the hole transport layer and the hole adjustment layer may each include one or more hole transport materials, and the hole transport materials may be selected from a carbazole multimer, carbazole connected triarylamine compounds or other types of compounds, and in particular may be selected from compounds shown below or any combination of them:

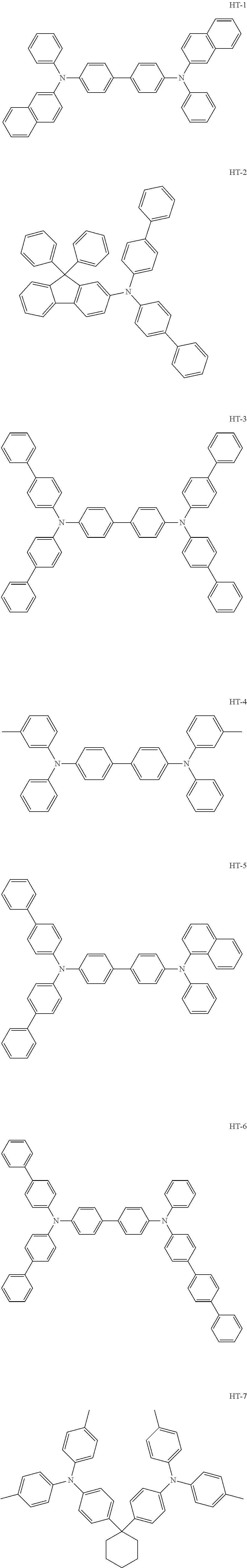

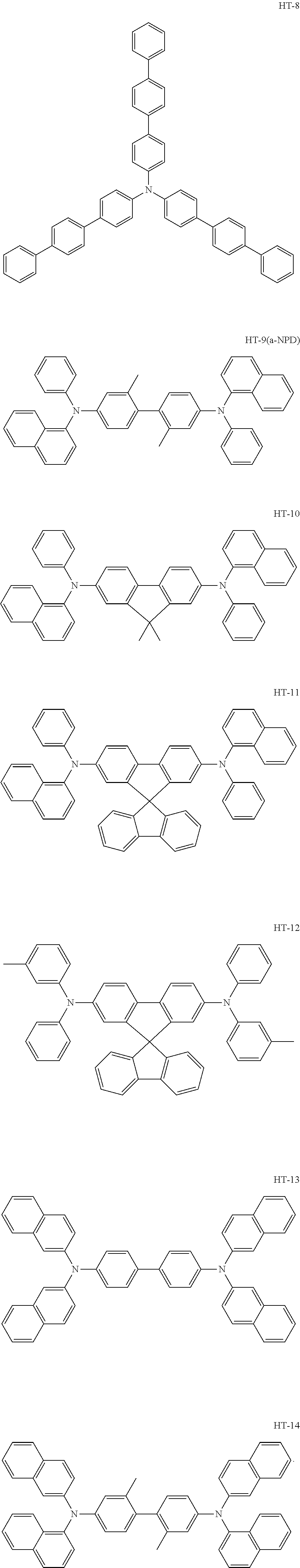

In one embodiment, the hole transport layer 321 consists of α-NPD.

In one embodiment, the hole adjustment layer 322 consists of HT-2.

Optionally, the hole injection layer 310 is further arranged between the anode 100 and the hole transport layer 321 to enhance the ability to inject holes to the hole transport layer 321. The hole injection layer 310 can be made of a benzidine derivative, a starburst arylamine compound, a phthalocyanine derivative or other materials, which is not specially limited in the disclosure. A material of the hole injection layer 310 is, for example, selected from the following compounds or any combination of them;

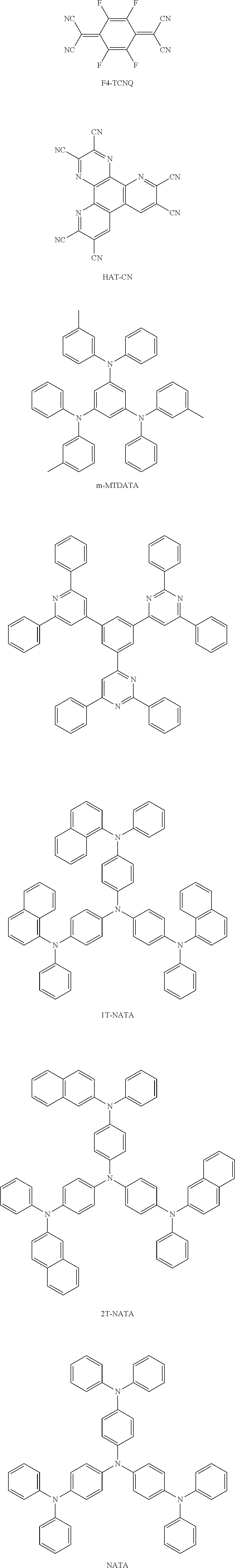

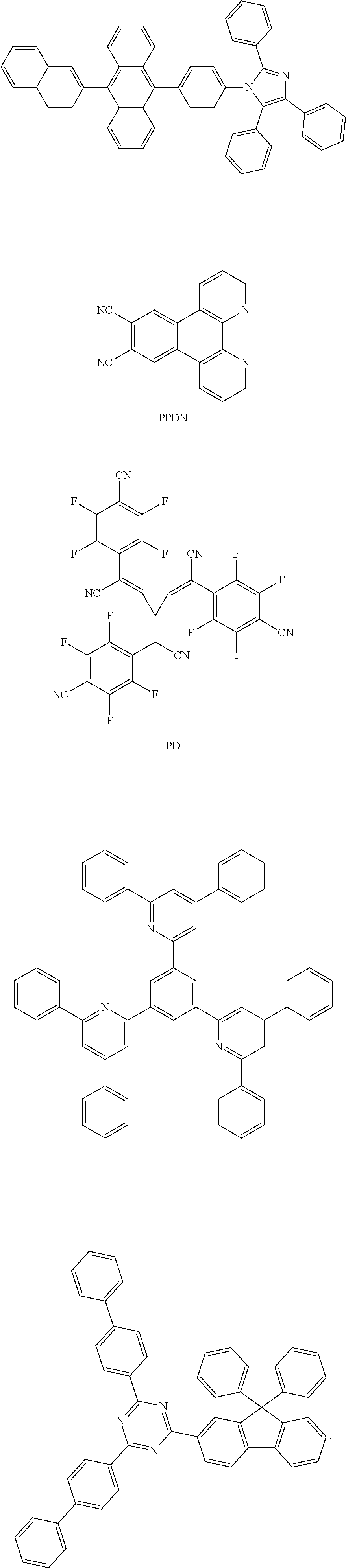

In one embodiment of the disclosure, the hole injection layer 310 consists of PD and α-NPD.

Optionally, the organic electroluminescent layer 330 may be composed of a single electroluminescent material, and may also include a host material and a dopant material. Optionally, the organic electroluminescent layer 330 is composed of the host material and the dopant material, holes injected into the organic electroluminescent layer 330 and electrons injected into the organic electroluminescent layer 330 can be recombined in the organic electroluminescent layer 330 to form excitons, the excitons transfer energy to the host material, the host material transfers energy to the dopant material, and then the dopant material can emit light.

The host material of the organic electroluminescent layer 330 may include a metal chelated compound, a distyryl derivative, an aromatic amine derivative, a dibenzofuran derivative, or other types of materials. The host material of the organic electroluminescent layer 330 may be one compound, or a combination of two or more compounds. Optionally, the host material includes the arylamine compound of the disclosure.

The dopant material of the organic electroluminescent layer 330 may be a compound having a condensed aryl ring or its derivative, a compound having a heteroaryl ring or its derivative, an aromatic amine derivative, or other materials, which is not specially limited in the disclosure. The dopant material is also referred to as a doping material or a dopant. The dopant material can be divided into fluorescent dopants and phosphorescent dopants according to luminescence types. Specific examples of the phosphorescent dopants include, but are not limited to,

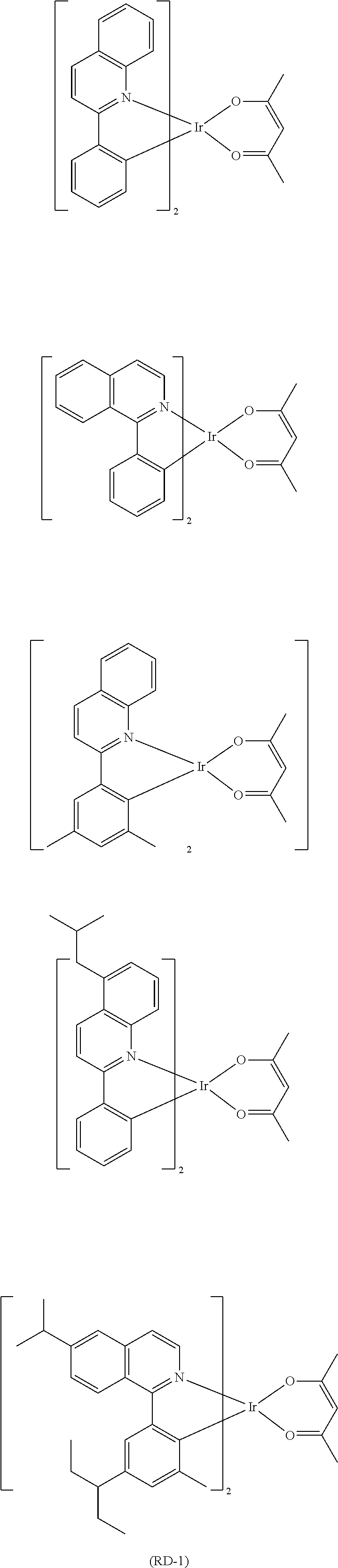

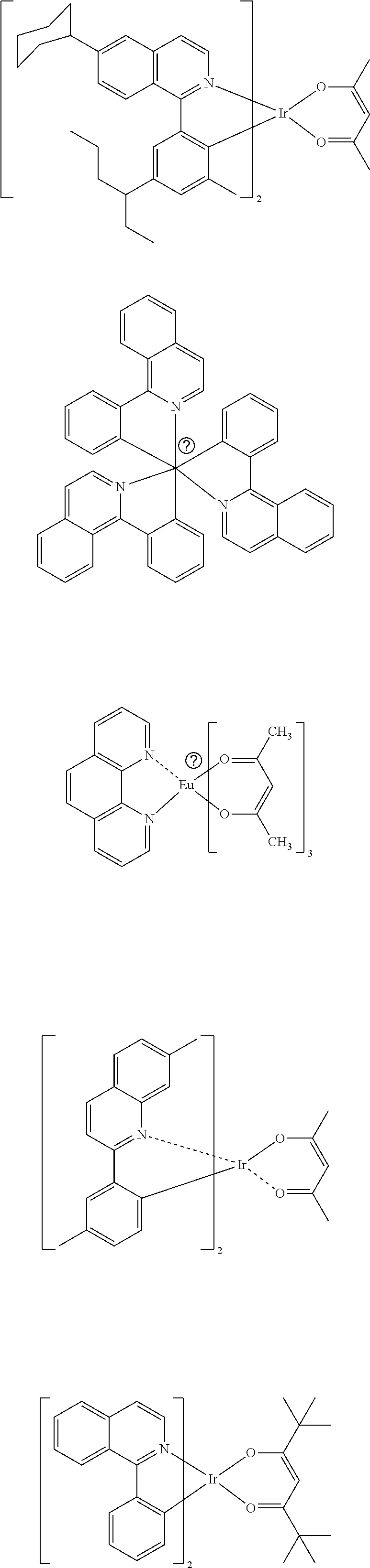

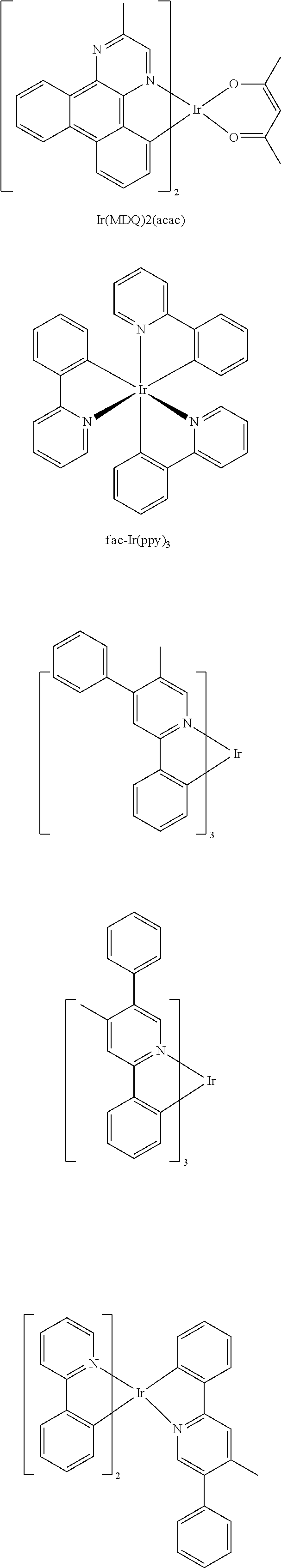

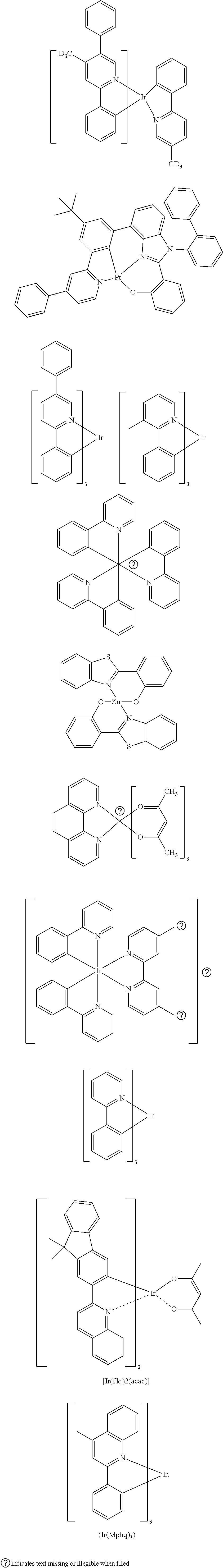

In one embodiment of the disclosure, the organic electroluminescent device is a red organic electroluminescent device. In one more specific embodiment, the host material of the organic electroluminescent layer 330 includes the arylamine compound of the disclosure. The dopant material may be, for example, RD-1.

The electron transport layer 340 may be of a single-layer structure or a multi-layer structure, and may include one or more electron transport materials, and the electron transport materials may be selected from, but are not limited to, LiQ, a benzimidazole derivative, an oxadiazole derivative, a quinoxaline derivative, or other electron transport materials, which are not particularly limited in the disclosure. A material of the electron transport layer 340 includes, but is not limited to, the following compounds:

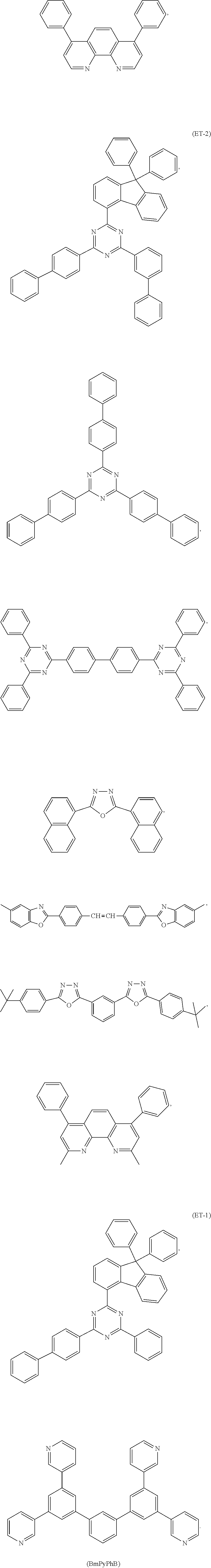

In one embodiment of the disclosure, the electron transport layer 340 consists of ET-1 and LiQ.

In the disclosure, the cathode 200 includes a cathode material, which is a material having a small work function that facilitates electron injection into the functional layer. Specific examples of the cathode material include, but are not limited to, metals such as magnesium, calcium, sodium, potassium, titanium, indium, yttrium, lithium, gadolinium, aluminum, silver, tin, and lead, or an alloy of them; or a multilayer material such as LiF/Al, Liq/Al, LiO₂/Al, LiF/Ca, LiF/Al, and BaF₂/Ca. Optionally, a metal electrode including magnesium and silver as the cathode is included.

Optionally, the electron injection layer 350 is further arranged between the cathode 200 and the electron transport layer 340 to enhance the ability to inject electrons into the electron transport layer 340. The electron injection layer 350 may include an inorganic material such as an alkali metal sulfide, and an alkali metal halide, or may include a complex of an alkali metal and an organic substance. In one embodiment of the disclosure, the electron injection layer 350 includes ytterbium (Yb).

According to a third aspect of the disclosure, provided is an electronic apparatus, including the organic electroluminescent device according to the second aspect of the disclosure.

According to one embodiment, as shown in FIG. 2, the electronic apparatus provided is an electronic apparatus 400 including the organic electroluminescent device described above. The electronic apparatus 400 may be, for example, a display device, a lighting device, an optical communication device, or other type of electronic devices, and may include, for example, but is not limited to, a computer screen, a mobile phone screen, a television, electronic paper, an emergency lighting lamp, an optical module, and the like.

A synthesis method of the arylamine compound of the disclosure is specifically illustrated below with reference to synthesis examples, but the disclosure is not limited in any way accordingly.

### Synthesis Examples

Those skilled in the art will recognize that chemical reactions described in the disclosure may be used to suitably prepare a number of arylamine compounds of the disclosure, and that other methods for preparing the compounds of the disclosure are considered to be within the scope of the disclosure. For example, the synthesis of those non-exemplified compounds according to the disclosure can be successfully accomplished by those skilled in the art by modification methods such as appropriately protecting interfering groups, by utilizing other known reagents other than those described in the disclosure, or by making some conventional modification of reaction conditions. Compounds of which synthesis methods are not mentioned in the disclosure are all commercially available raw material products.

### Synthesis of Sub-a1:

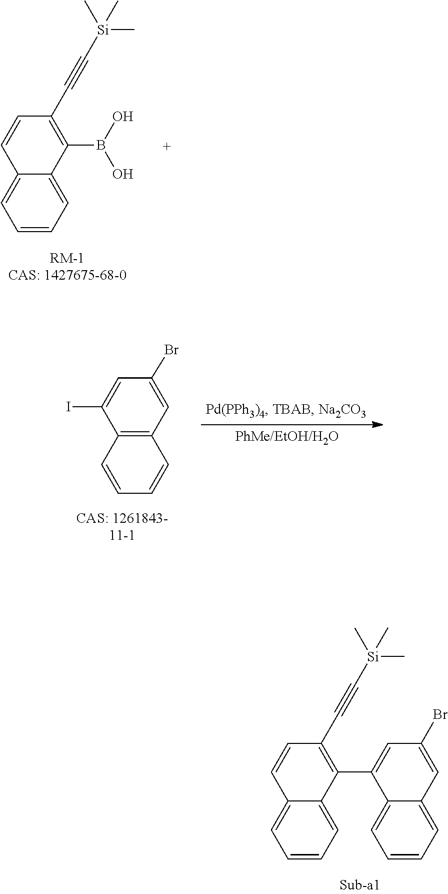 [SMILES: C[Si](C)(C)C#Cc1ccc2ccccc2c1B(O)O ; Brc1cc(I)c2ccccc2c1 ; C[Si](C)(C)C#Cc1ccc2ccccc2c1-c1cc(Br)cc2ccccc12]

RM-1 (CAS: 1427675-68-0, 13.41 g, 50 mmol), 1-iodo-3-bromonaphthalene (16.64 g, 50 mmol), tetrakis(triphenylphosphine) palladium (0.58 g, 0.5 mmol), tetrabutylammonium bromide (1.61 g, 5 mmol), anhydrous sodium carbonate (10.6 g, 100 mmol), toluene (140 mL), anhydrous ethanol (35 mL) and deionized water (35 mL) were sequentially added into a 500 mL three-necked flask under a nitrogen atmosphere, stirring and heating were started, heating was performed to reflux, and a reaction was carried out for 16 h. After the reaction mixture was cooled to room temperature, the resulting reaction solution was extracted with dichloromethane (100 mL×3 times), organic phases were mixed, dried over anhydrous magnesium sulfate, and filtered, and the filtrate was concentrated in vacuum to remove solvent and obtain a crude product. The crude product was purified by silica gel column chromatography with n-heptane as a mobile phase, to obtain Sub-a1 (13.31 g, yield: 62%) as a white solid.

With reference to the synthesis method of Sub-a1, Sub-a2 to Sub-a4 were synthesized by using a reactant A shown in Table 1 instead of 1-iodo-3-bromonaphthalene.

TABLE 1
Synthesis of Sub-a2 to Sub-a4

| Sub-a No. | Reactant A | Structure of Sub-a | Yield (%) |
|---|---|---|---|
| Sub-a2 | 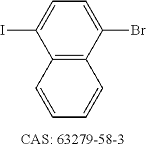 [SMILES: Brc1ccc(I)c2ccccc12] | 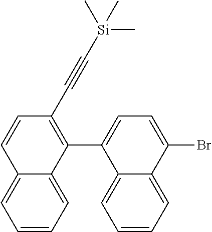 [SMILES: C[Si](C)(C)C#Cc1ccc2ccccc2c1-c1ccc(Br)c2ccccc12] | 64 |
| Sub-a3 | 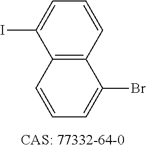 [SMILES: Brc1cccc2c(I)cccc12] | 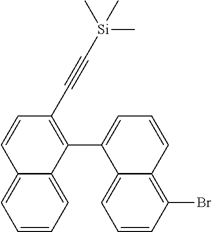 [SMILES: C[Si](C)(C)C#Cc1ccc2ccccc2c1-c1cccc2c(Br)cccc12] | 66 |
| Sub-a4 | 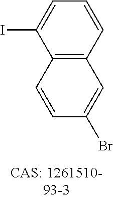 [SMILES: Brc1ccc2c(I)cccc2c1] | 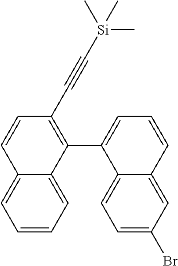 [SMILES: C[Si](C)(C)C#Cc1ccc2ccccc2c1-c1cccc2cc(Br)ccc12] | 65 |

### Synthesis of Sub-b1:

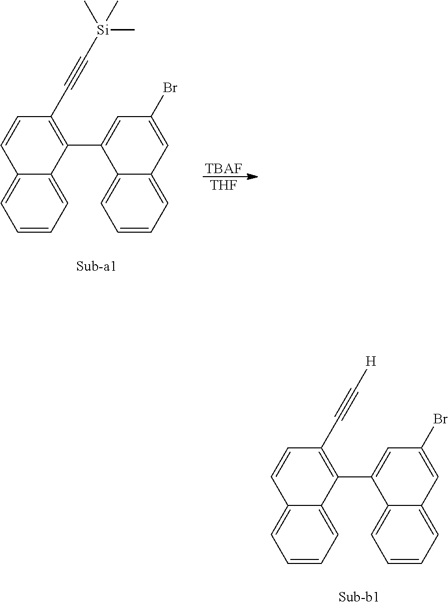

Sub-a1 (21.47 g, 50 mmol), tetrabutylammonium fluoride (1.0 M in tetrahydrofuran, 150 mL) and deionized water (150 mL) were sequentially added into a 500 mL three-neck flask under a nitrogen atmosphere, and a reaction was carried out under stirring at room temperature for 2 h. After the reaction was completed, the resulting reaction solution was extracted with dichloromethane (50 mL×3 times), organic phases were mixed, dried over anhydrous magnesium sulfate, and filtered, and the filtrate was concentrated in vacuum to remove solvent and obtain a crude product. The crude product was purified by silica gel column chromatography with n-heptane as a mobile phase, to obtain Sub-b1 (15.72 g, yield: 88%) as a white solid.

With reference to the synthesis method of Sub-b1, Sub-b2 to Sub-b4 were synthesized by using a reactant B shown in Table 2 instead of Sub-a1.

TABLE 2
Synthesis of Sub-b2 to Sub-b4

| Sub-b No. | Reactant B | Structure of Sub-b | Yield (%) |
|---|---|---|---|
| Sub-b2 | 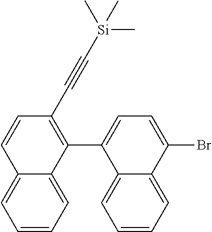 [SMILES: C[Si](C)(C)C#Cc1ccc2ccccc2c1-c1ccc(Br)c2ccccc12] | 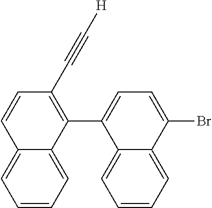 [SMILES: C#Cc1ccc2ccccc2c1-c1ccc(Br)c2ccccc12] | 89 |
| Sub-b3 | 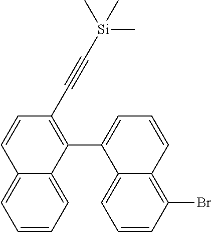 [SMILES: C[Si](C)(C)C#Cc1ccc2ccccc2c1-c1cccc2c(Br)cccc12] | 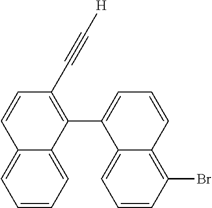 [SMILES: C#Cc1ccc2ccccc2c1-c1cccc2c(Br)cccc12] | 90 |
| Sub-b4 | 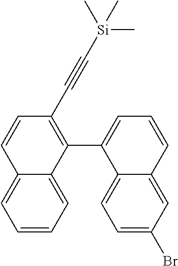 [SMILES: C[Si](C)(C)C#Cc1ccc2ccccc2c1-c1cccc2cc(Br)ccc12] | 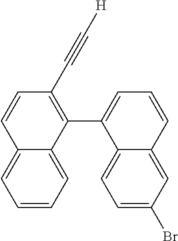 [SMILES: C#Cc1ccc2ccccc2c1-c1cccc2cc(Br)ccc12] | 87 |

### Synthesis of Sub-c1:

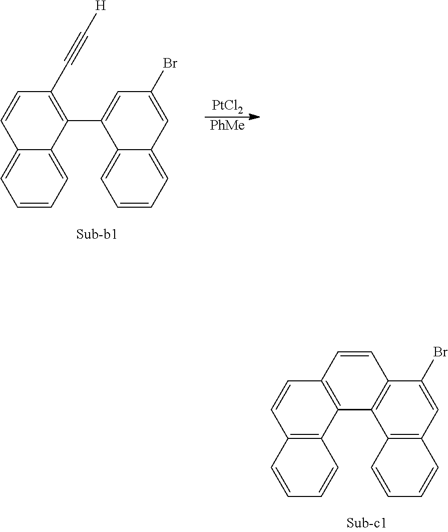

Sub-b1 (17.86 g, 50 mmol), platinum dichloride (0.916 g, 0.66 g, 2.5 mmol) and toluene (180 mL) were sequentially added into a 500 mL three-neck flask under a nitrogen atmosphere, heating was performed to reflux, and a reaction was carried out under stirring for 24 h. After the system was cooled to room temperature, the resulting reaction solution was extracted with dichloromethane (100 mL×3 times), organic phases were mixed, dried over anhydrous magnesium sulfate, and filtered, and the filtrate was concentrated in vacuum to remove solvent and obtain a crude product. The crude product was purified by silica gel column chromatography with n-heptane as a mobile phase, to obtain Sub-c1 (13.93 g, yield: 78%) as a white solid.

With reference to the synthesis method of Sub-c1, Sub-c2 to Sub-c4 were synthesized by using a reactant C shown in Table 3 instead of Sub-b1.

TABLE 3
Synthesis of Sub-c2 to Sub-c4

| Sub-c No. | Reactant C | Structure of Sub-c | Yield (%) |
|---|---|---|---|
| Sub-c2 | 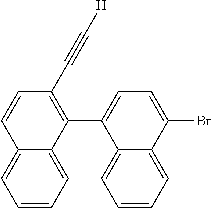 [SMILES: C#Cc1ccc2ccccc2c1-c1ccc(Br)c2ccccc12] | 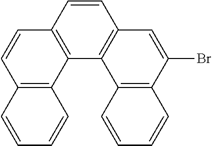 [SMILES: Brc1cc2ccc3ccc4ccccc4c3c2c2ccccc12] | 61 |
| Sub-c3 | 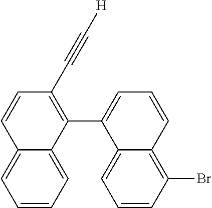 [SMILES: C#Cc1ccc2ccccc2c1-c1cccc2c(Br)cccc12] | 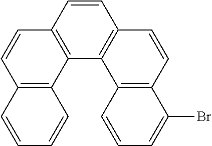 [SMILES: Brc1cccc2c1ccc1ccc3ccc4ccccc4c3c12] | 71 |
| Sub-c4 | 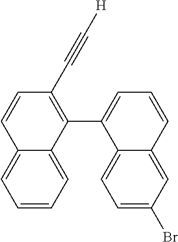 [SMILES: C#Cc1ccc2ccccc2c1-c1cccc2cc(Br)ccc12] | 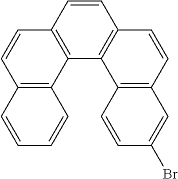 [SMILES: Brc1ccc2c(ccc3ccc4ccc5ccccc5c4c32)c1] | 60 |

### Synthesis of Sub-c5:

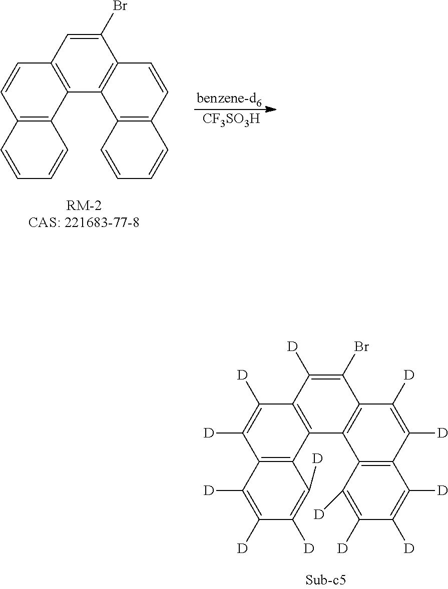

A compound RM-2 (CAS: 221683-77-8, 8.93 g, 25 mmol) and 200 mL of benzene-D₆ were added into a 100 mL three-necked flask under a nitrogen atmosphere, heating was performed to 60° C., trifluoromethanesulfonic acid (22.51 g, 150 mmol) was added into the three-necked flask, heating was continued to be performed to boiling, and a reaction was carried out under stirring for 24 h. After the reaction system was cooled to room temperature, 50 mL of deuterium oxide was added into the three-necked flask, and after stirring for 10 min, the reaction solution was neutralized by adding a saturated aqueous K₃PO₄ solution. An organic layer was extracted with dichloromethane (50 mL×3 times), organic phases were mixed, dried over anhydrous magnesium sulfate, filtered, and the filtrate was concentrated in vacuum to remove solvent and obtain a crude product. The crude product was purified by silica gel column chromatography with a mixture of n-heptane and dichloromethane as a mobile phase, to obtain Sub-c5 (5.37 g, yield: 58%) as a white solid.

### Synthesis of Sub-d1:

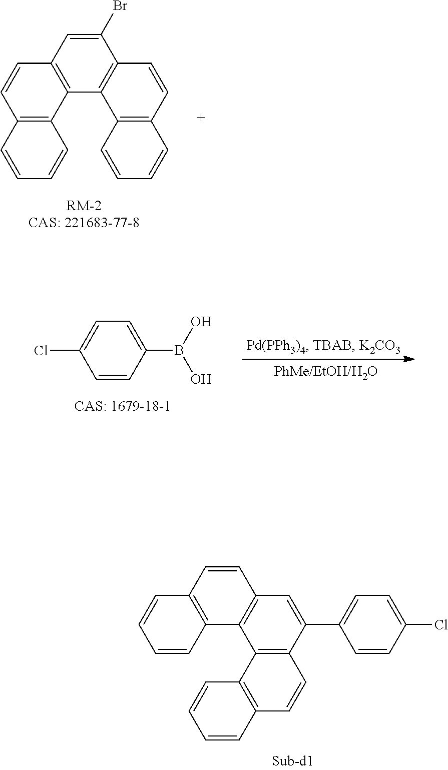 [SMILES: Brc1cc2ccc3ccccc3c2c2c1ccc1ccccc12 ; OB(O)c1ccc(Cl)cc1 ; Clc1ccc(-c2cc3ccc4ccccc4c3c3c2ccc2ccccc23)cc1]

RM-2 (CAS: 221683-77-8, 17.86 g, 50 mmol), 4-chlorophenylboronic acid (8.60 g, 55 mmol), tetrakis(triphenylphosphine) palladium (0.58 g, 0.5 mmol), tetrabutylammonium bromide (1.61 g, 5 mmol), anhydrous potassium carbonate (13.82 g, 100 mmol), toluene (180 mL), anhydrous ethanol (45 mL) and deionized water (45 mL) were sequentially added into a 500 ml three-necked flask under a nitrogen atmosphere, stirring and heating were started, heating was performed to reflux, and a reaction was carried out for 16 h. After the system was cooled to room temperature, the resulting reaction solution was extracted with dichloromethane (100 mL×3 times), organic phases were mixed, dried over anhydrous magnesium sulfate, and filtered, and the filtrate was concentrated in vacuum to remove solvent and obtain a crude product. The crude product was purified by silica gel column chromatography with a mixture of n-heptane and ethyl acetate as a mobile phase, to obtain Sub-d1 (15.94 g, yield: 82%) as a white solid.

With reference to the synthesis method of Sub-d1, Sub-d2 to Sub-d23 were synthesized by using a reactant D shown in Table 4 instead of RM-2, and using a reactant E shown in Table 4 instead of 4-chlorophenylboronic acid.

TABLE 4
Synthesis of Sub-d2 to Sub-d23

| Sub-d No. | Reactant D | Reactant E |
|---|---|---|
| Sub-d2 | 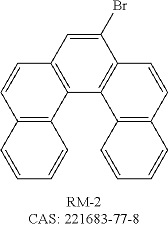 [SMILES: Brc1cc2ccc3ccccc3c2c2c1ccc1ccccc12] | 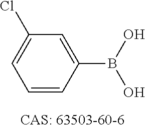 |
| Sub-d3 | 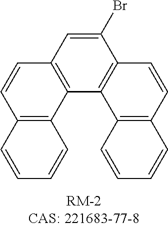 [SMILES: Brc1cc2ccc3ccccc3c2c2c1ccc1ccccc12] | 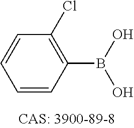 |
| Sub-d4 | 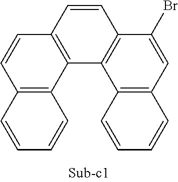 [SMILES: Brc1cc2ccccc2c2c1ccc1ccc3ccccc3c12] | 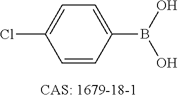 |
| Sub-d5 | 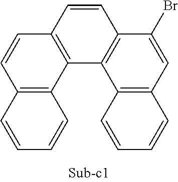 [SMILES: Brc1cc2ccccc2c2c1ccc1ccc3ccccc3c12] | 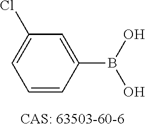 |
| Sub-d6 | 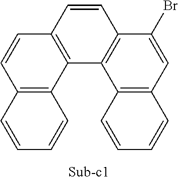 [SMILES: Brc1cc2ccccc2c2c1ccc1ccc3ccccc3c12] | 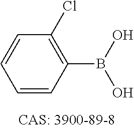 |
| Sub-d7 | 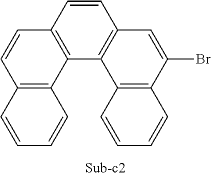 [SMILES: Brc1cc2ccc3ccc4ccccc4c3c2c2ccccc12] | 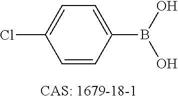 |
| Sub-d8 | 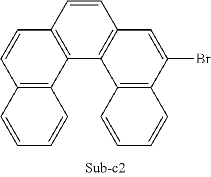 [SMILES: Brc1cc2ccc3ccc4ccccc4c3c2c2ccccc12] | 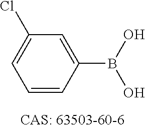 |
| Sub-d9 | 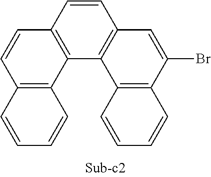 [SMILES: Brc1cc2ccc3ccc4ccccc4c3c2c2ccccc12] | 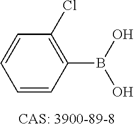 |
| Sub-d10 | 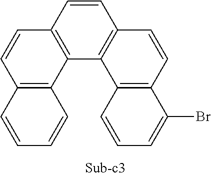 [SMILES: Brc1cccc2c1ccc1ccc3ccc4ccccc4c3c12] | 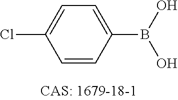 |
| Sub-d11 | 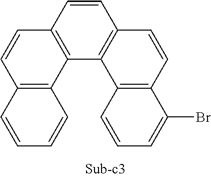 [SMILES: Brc1cccc2c1ccc1ccc3ccc4ccccc4c3c12] | 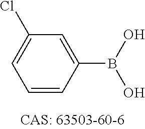 |
| Sub-d12 | 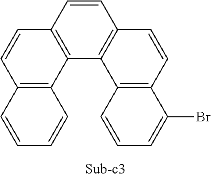 [SMILES: Brc1cccc2c1ccc1ccc3ccc4ccccc4c3c12] | 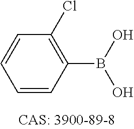 |
| Sub-d13 | 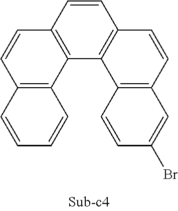 [SMILES: Brc1ccc2c(ccc3ccc4ccc5ccccc5c4c32)c1] | 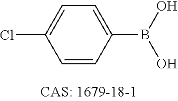 |
| Sub-d14 | 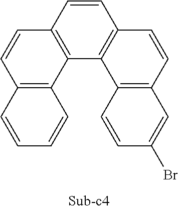 [SMILES: Brc1ccc2c(ccc3ccc4ccc5ccccc5c4c32)c1] | 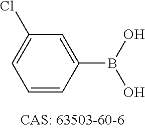 |
| Sub-d15 | 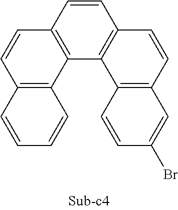 [SMILES: Brc1ccc2c(ccc3ccc4ccc5ccccc5c4c32)c1] | 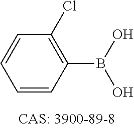 |
| Sub-d16 | 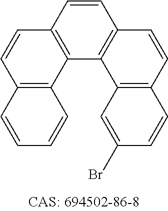 [SMILES: Brc1ccc2ccc3ccc4ccc5ccccc5c4c3c2c1] | 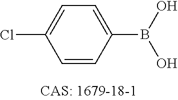 |
| Sub-d17 | 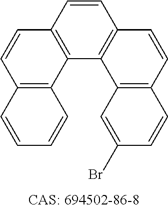 [SMILES: Brc1ccc2ccc3ccc4ccc5ccccc5c4c3c2c1] | 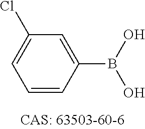 |
| Sub-d18 | 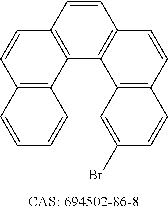 [SMILES: Brc1ccc2ccc3ccc4ccc5ccccc5c4c3c2c1] | 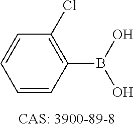 |
| Sub-d19 | 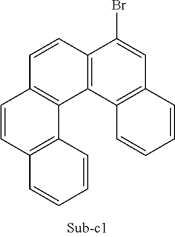 [SMILES: Brc1cc2ccccc2c2c1ccc1ccc3ccccc3c12] | 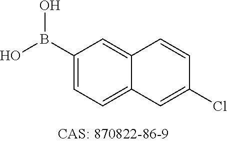 [SMILES: OB(O)c1ccc2cc(Cl)ccc2c1] |
| Sub-d20 | 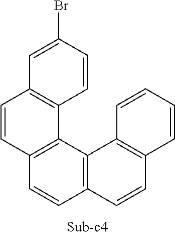 [SMILES: Brc1ccc2c(ccc3ccc4ccc5ccccc5c4c32)c1] | 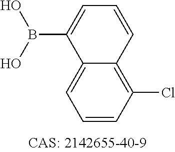 [SMILES: OB(O)c1cccc2c(Cl)cccc12] |
| Sub-d21 | 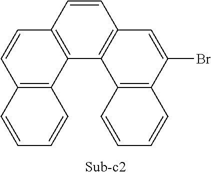 [SMILES: Brc1cc2ccc3ccc4ccccc4c3c2c2ccccc12] | 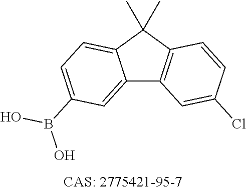 [SMILES: CC1(C)c2ccc(Cl)cc2-c2cc(B(O)O)ccc21] |
| Sub-d22 | 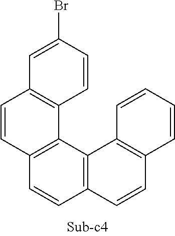 [SMILES: Brc1ccc2c(ccc3ccc4ccc5ccccc5c4c32)c1] | 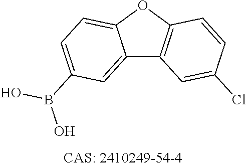 [SMILES: OB(O)c1ccc2oc3ccc(Cl)cc3c2c1] |
| Sub-d23 | 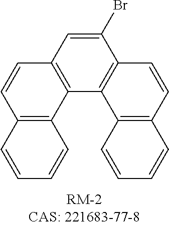 [SMILES: Brc1cc2ccc3ccccc3c2c2c1ccc1ccccc12] | 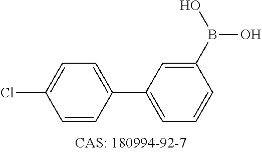 [SMILES: OB(O)c1cccc(-c2ccc(Cl)cc2)c1] |

| Sub-d No. | Structure of Sub-d | Yield % |
|---|---|---|
| Sub-d2 | 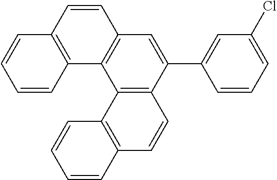 [SMILES: Clc1cccc(-c2cc3ccc4ccccc4c3c3c2ccc2ccccc23)c1] | 80 |
| Sub-d3 | 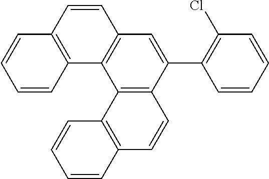 [SMILES: Clc1ccccc1-c1cc2ccc3ccccc3c2c2c1ccc1ccccc12] | 76 |
| Sub-d4 | 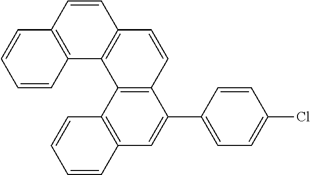 [SMILES: Clc1ccc(-c2cc3ccccc3c3c2ccc2ccc4ccccc4c23)cc1] | 72 |
| Sub-d5 | 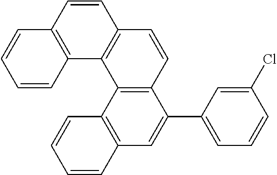 [SMILES: Clc1cccc(-c2cc3ccccc3c3c2ccc2ccc4ccccc4c23)c1] | 73 |
| Sub-d6 | 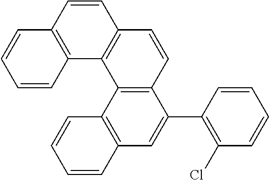 [SMILES: Clc1ccccc1-c1cc2ccccc2c2c1ccc1ccc3ccccc3c12] | 70 |
| Sub-d7 | 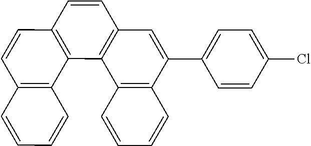 [SMILES: Clc1ccc(-c2cc3ccc4ccc5ccccc5c4c3c3ccccc23)cc1] | 85 |
| Sub-d8 | 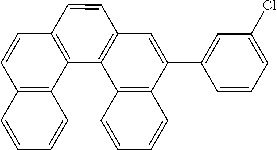 [SMILES: Clc1cccc(-c2cc3ccc4ccc5ccccc5c4c3c3ccccc23)c1] | 76 |
| Sub-d9 | 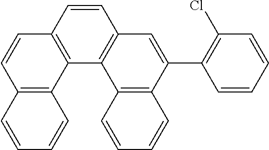 [SMILES: Clc1ccccc1-c1cc2ccc3ccc4ccccc4c3c2c2ccccc12] | 72 |
| Sub-d10 | 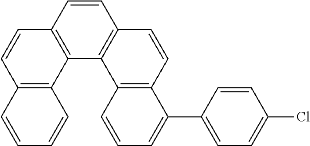 [SMILES: Clc1ccc(-c2cccc3c2ccc2ccc4ccc5ccccc5c4c23)cc1] | 79 |
| Sub-d11 | 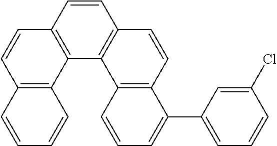 [SMILES: Clc1cccc(-c2cccc3c2ccc2ccc4ccc5ccccc5c4c23)c1] | 81 |
| Sub-d12 | 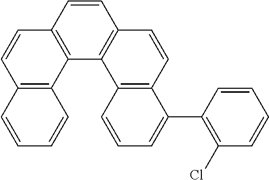 [SMILES: Clc1ccccc1-c1cccc2c1ccc1ccc3ccc4ccccc4c3c12] | 73 |
| Sub-d13 | 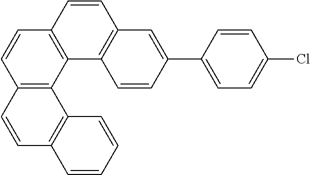 [SMILES: Clc1ccc(-c2ccc3c(ccc4ccc5ccc6ccccc6c5c43)c2)cc1] | 84 |
| Sub-d14 | 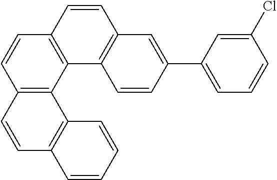 [SMILES: Clc1cccc(-c2ccc3c(ccc4ccc5ccc6ccccc6c5c43)c2)c1] | 85 |
| Sub-d15 | 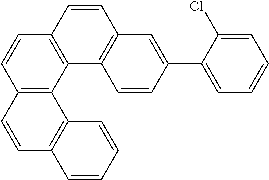 [SMILES: Clc1ccccc1-c1ccc2c(ccc3ccc4ccc5ccccc5c4c32)c1] | 75 |
| Sub-d16 | 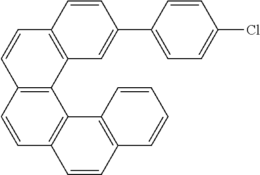 [SMILES: Clc1ccc(-c2ccc3ccc4ccc5ccc6ccccc6c5c4c3c2)cc1] | 80 |
| Sub-d17 | 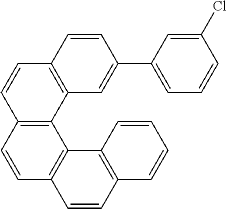 [SMILES: Clc1cccc(-c2ccc3ccc4ccc5ccc6ccccc6c5c4c3c2)c1] | 83 |
| Sub-d18 | 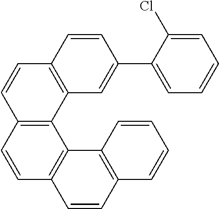 [SMILES: Clc1ccccc1-c1ccc2ccc3ccc4ccc5ccccc5c4c3c2c1] | 71 |
| Sub-d19 | 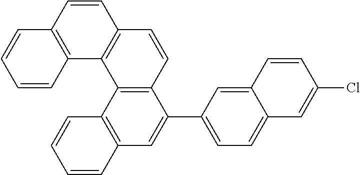 [SMILES: Clc1ccc2cc(-c3cc4ccccc4c4c3ccc3ccc5ccccc5c34)ccc2c1] | 83 |
| Sub-d20 | 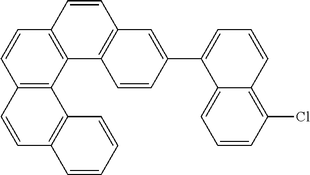 [SMILES: Clc1cccc2c(-c3ccc4c(ccc5ccc6ccc7ccccc7c6c54)c3)cccc12] | 79 |
| Sub-d21 | 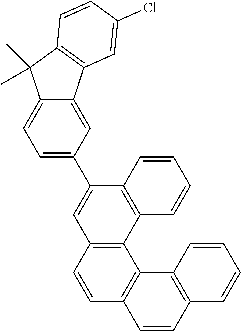 [SMILES: CC1(C)c2ccc(Cl)cc2-c2cc(-c3cc4ccc5ccc6ccccc6c5c4c4ccccc34)ccc21] | 73 |
| Sub-d22 | 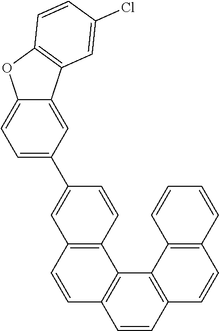 [SMILES: Clc1ccc2oc3ccc(-c4ccc5c(ccc6ccc7ccc8ccccc8c7c65)c4)cc3c2c1] | 74 |
| Sub-d23 | 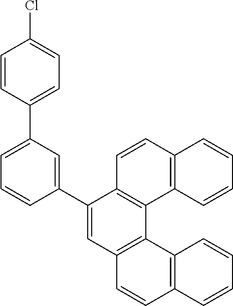 [SMILES: Clc1ccc(-c2cccc(-c3cc4ccc5ccccc5c4c4c3ccc3ccccc34)c2)cc1] | 76 |

### Synthesis of Sub-e1:

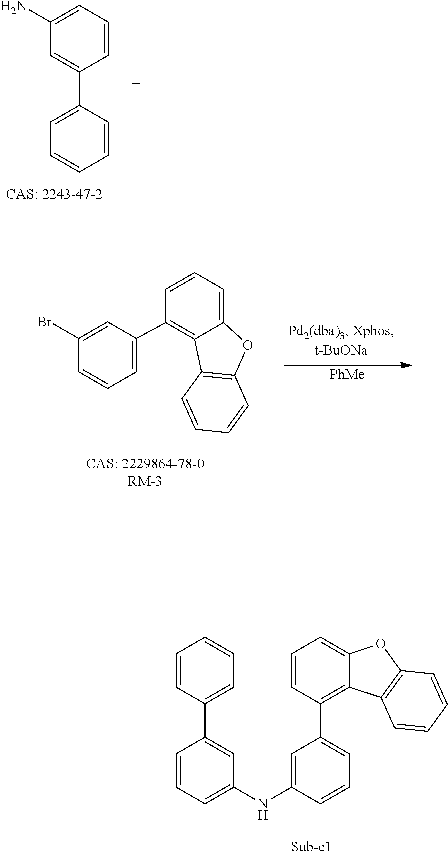 [SMILES: Nc1cccc(-c2ccccc2)c1 ; Brc1cccc(-c2cccc3oc4ccccc4c23)c1 ; "PhMe" ; c1ccc(-c2cccc(Nc3cccc(-c4cccc5oc6ccccc6c45)c3)c2)cc1]

3-Aminobiphenyl (9.31 g, 55 mmol), RM-3 (CAS: 2229864-78-0, 16.15 g, 50 mmol), tris (dibenzyleneacetone) dipalladium (0.916 g, 1 mmol), (2-dicyclohexylphosphino-2′,4′,6′-triisopropylbiphenyl) (0.95 g, 2 mmol), sodium tert-butoxide (9.61 g, 100 mmol) and toluene (160 mL) were sequentially added into a 250 mL three-necked flask under a nitrogen atmosphere, heating was performed to reflux, and a reaction was carried out under stirring overnight. After the system was cooled to room temperature, the reaction solution was poured into 250 mL of deionized water, well stirring was performed for 30 min, suction filtration was performed, and the obtained filter cake was subjected to drip washing with deionized water to be neutral, and then washed with absolute ethanol (200 mL) to remove water to obtain a crude product; and the crude product was purified by silica gel column chromatography with a mixture of dichloromethane and n-heptane as a mobile phase, to give Sub-e1 (15.0 g; yield: 73%) as a white solid.

With reference to the synthesis method of Sub-e1, Sub-e2 to Sub-e7 were synthesized by using a reactant F shown in Table 5 instead of 3-aminobiphenyl, and using a reactant G shown in Table 5 instead of RM-3.

TABLE 5
Synthesis of Sub-e2 to Sub-e7

| Sub-e No. | Reactant F | Reactant G |
|---|---|---|
| Sub-e2 | 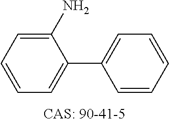 [SMILES: Nc1ccccc1-c1ccccc1] | 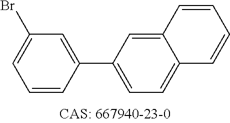 [SMILES: Brc1cccc(-c2ccc3ccccc3c2)c1] |
| Sub-e3 | 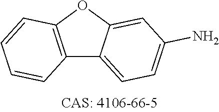 [SMILES: Nc1ccc2c(c1)oc1ccccc12] | 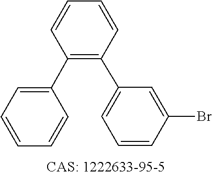 [SMILES: Brc1cccc(-c2ccccc2-c2ccccc2)c1] |
| Sub-e4 | 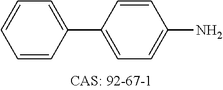 [SMILES: Nc1ccc(-c2ccccc2)cc1] | 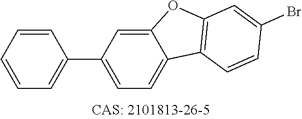 [SMILES: Brc1ccc2c(c1)oc1cc(-c3ccccc3)ccc12] |
| Sub-e5 | 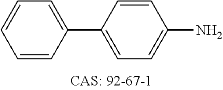 [SMILES: Nc1ccc(-c2ccccc2)cc1] | 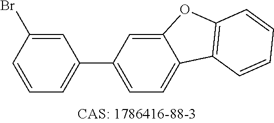 [SMILES: Brc1cccc(-c2ccc3c(c2)oc2ccccc23)c1] |
| Sub-e6 | 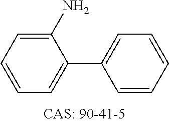 [SMILES: Nc1ccccc1-c1ccccc1] | 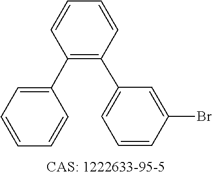 [SMILES: Brc1cccc(-c2ccccc2-c2ccccc2)c1] |
| Sub-e7 | 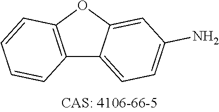 [SMILES: Nc1ccc2c(c1)oc1ccccc12] | 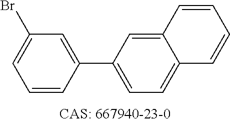 [SMILES: Brc1cccc(-c2ccc3ccccc3c2)c1] |

| Sub-e No. | Structure of Sub-e | Yield % |
|---|---|---|
| Sub-e2 | 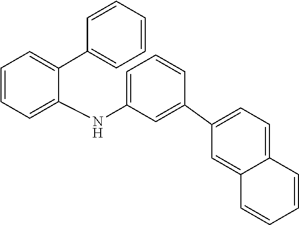 [SMILES: c1ccc(-c2ccccc2Nc2cccc(-c3ccc4ccccc4c3)c2)cc1] | 62 |
| Sub-e3 | 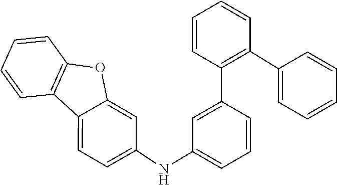 [SMILES: c1ccc(-c2ccccc2-c2cccc(Nc3ccc4c(c3)oc3ccccc34)c2)cc1] | 66 |
| Sub-e4 | 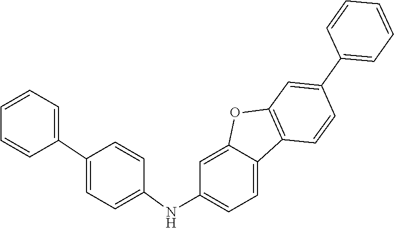 [SMILES: c1ccc(-c2ccc(Nc3ccc4c(c3)oc3cc(-c5ccccc5)ccc34)cc2)cc1] | 72 |
| Sub-e5 | 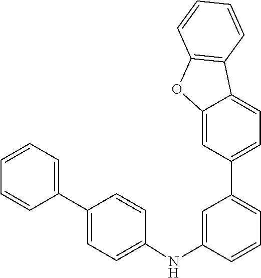 [SMILES: c1ccc(-c2ccc(Nc3cccc(-c4ccc5c(c4)oc4ccccc45)c3)cc2)cc1] | 69 |
| Sub-e6 | 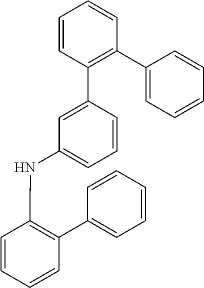 [SMILES: c1ccc(-c2ccccc2Nc2cccc(-c3ccccc3-c3ccccc3)c2)cc1] | 61 |
| Sub-e7 | 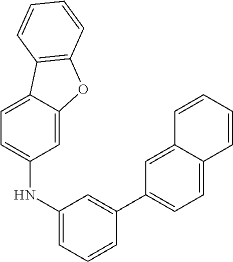 [SMILES: c1cc(Nc2ccc3c(c2)oc2ccccc23)cc(-c2ccc3ccccc3c2)c1] | 75 |

### Synthesis of Compound 6:

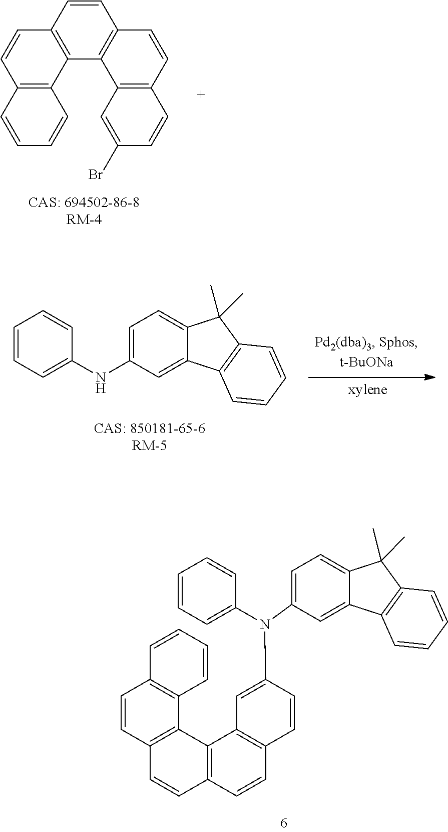 [SMILES: Brc1ccc2ccc3ccc4ccc5ccccc5c4c3c2c1 ; CC1(C)c2ccccc2-c2cc(Nc3ccccc3)ccc21 ; CC1(C)c2ccccc2-c2cc(N(c3ccccc3)c3ccc4ccc5ccc6ccc7ccccc7c6c5c4c3)ccc21]

RM-4 (CAS: 694502-86-8, 10.72 g, 30 mmol), RM-5 (CAS: 850181-65-6, 9.42 g, 33 mmol), tris(dibenzyleneacetone) dipalladium (0.55 g, 0.6 mmol), (2-dicyclohexylphosphino-2′, 6′-dimethoxybiphenyl (0.49 g, 1.2 mmol), sodium tert-butoxide (5.77 g, 60 mmol) and xylene (120 mL) were sequentially added into a 250 mL three-necked flask under a nitrogen atmosphere, heating was performed to reflux, and a reaction was carried out under stirring overnight; after the system was cooled to room temperature, the resulting reaction solution was extracted with dichloromethane (100 mL×3 times), organic phases were mixed, dried over anhydrous magnesium sulfate, and filtered, and the filtrate was concentrated in vacuum to remove solvent and obtain a crude product; and the crude product was purified by silica gel column chromatography with a mixture of dichloromethane and n-heptane as a mobile phase, to obtain a compound 6 (12.97 g, yield: 77%, m/z=600.2 [M+H]⁺) as a white solid.

Referring to the synthesis of the compound 6, the compounds of the disclosure in Table 6 were synthesized by using a reactant H shown in Table 6 instead of RM-4, and using a reactant J shown in Table 6 instead of RM-5.

TABLE 6
Synthesis of compounds of the present disclosure

| | | | m/z | |
|---|---|---|---|---|
| | | Compound structure | ([M + | Yield/ |
| Reactant H | Reactant J | and No. | H]⁺) | % |
| 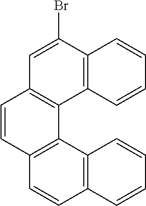 [SMILES: Brc1cc2ccc3ccc4ccccc4c3c2c2ccccc12] | 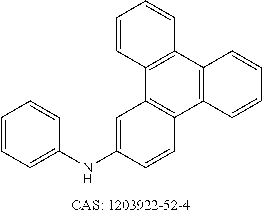 [SMILES: c1ccc(Nc2ccc3c4ccccc4c4ccccc4c3c2)cc1] | 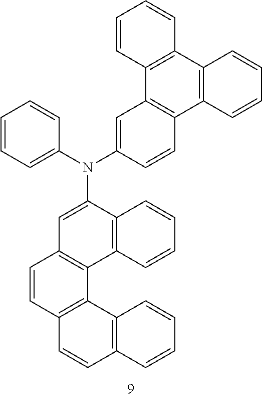 [SMILES: c1ccc(N(c2ccc3c4ccccc4c4ccccc4c3c2)c2cc3ccc4ccc5ccccc5c4c3c3ccccc23)cc1] | 596.2 | 69 |
| 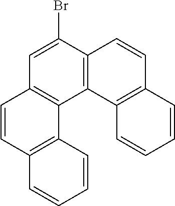 [SMILES: Brc1cc2ccc3ccccc3c2c2c1ccc1ccccc12] | 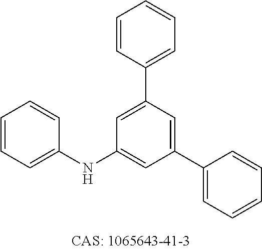 [SMILES: c1ccc(Nc2cc(-c3ccccc3)cc(-c3ccccc3)c2)cc1] | 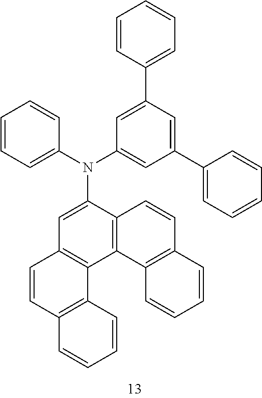 [SMILES: c1ccc(-c2cc(-c3ccccc3)cc(N(c3ccccc3)c3cc4ccc5ccccc5c4c4c3ccc3ccccc34)c2)cc1] | 598.3 | 65 |
| 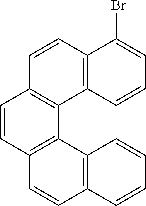 [SMILES: Brc1cccc2c1ccc1ccc3ccc4ccccc4c3c12] | 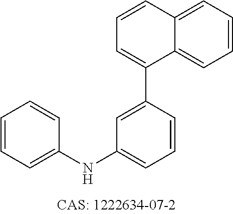 [SMILES: c1ccc(Nc2cccc(-c3cccc4ccccc34)c2)cc1] | 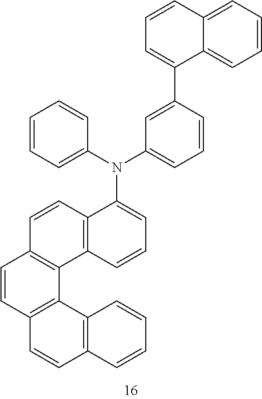 [SMILES: c1ccc(N(c2cccc(-c3cccc4ccccc34)c2)c2cccc3c2ccc2ccc4ccc5ccccc5c4c23)cc1] | 572.2 | 72 |
| 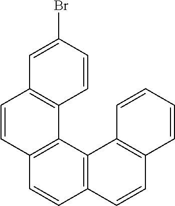 [SMILES: Brc1ccc2c(ccc3ccc4ccc5ccccc5c4c32)c1] | 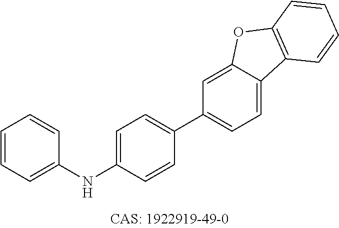 [SMILES: c1ccc(Nc2ccc(-c3ccc4c(c3)oc3ccccc34)cc2)cc1] | 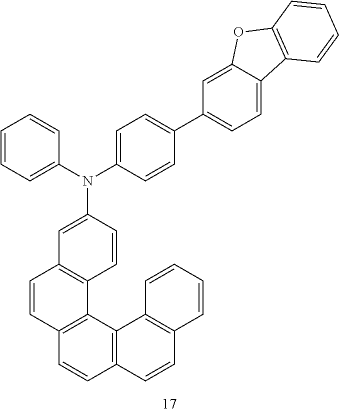 [SMILES: c1ccc(N(c2ccc(-c3ccc4c(c3)oc3ccccc34)cc2)c2ccc3c(ccc4ccc5ccc6ccccc6c5c43)c2)cc1] | 612.2 | 68 |
| 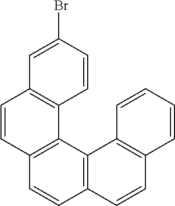 [SMILES: Brc1ccc2c(ccc3ccc4ccc5ccccc5c4c32)c1] | 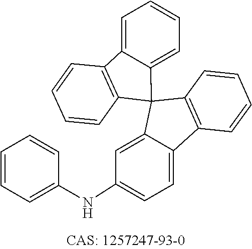 [SMILES: c1ccc(Nc2ccc3c(c2)C2(c4ccccc4-c4ccccc42)c2ccccc2-3)cc1] | 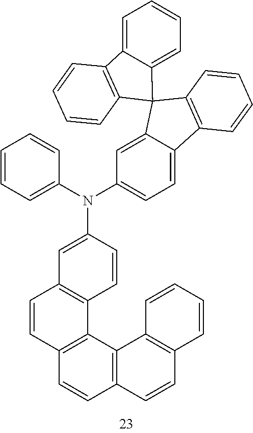 [SMILES: c1ccc(N(c2ccc3c(c2)C2(c4ccccc4-c4ccccc42)c2ccccc2-3)c2ccc3c(ccc4ccc5ccc6ccccc6c5c43)c2)cc1] | 684.3 | 71 |
| 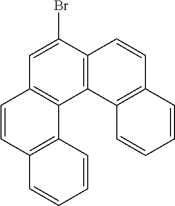 [SMILES: Brc1cc2ccc3ccccc3c2c2c1ccc1ccccc12] | 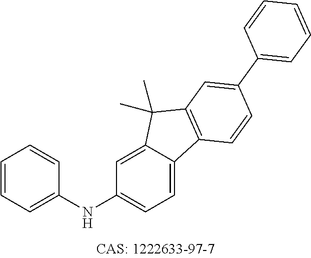 [SMILES: CC1(C)c2cc(Nc3ccccc3)ccc2-c2ccc(-c3ccccc3)cc21] | 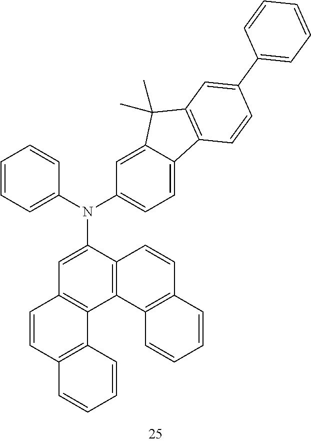 [SMILES: CC1(C)c2cc(-c3ccccc3)ccc2-c2ccc(N(c3ccccc3)c3cc4ccc5ccccc5c4c4c3ccc3ccccc34)cc21] | 638.3 | 69 |
| 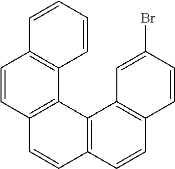 [SMILES: Brc1ccc2ccc3ccc4ccc5ccccc5c4c3c2c1] | 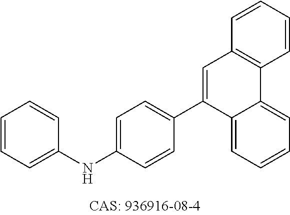 [SMILES: c1ccc(Nc2ccc(-c3cc4ccccc4c4ccccc34)cc2)cc1] | 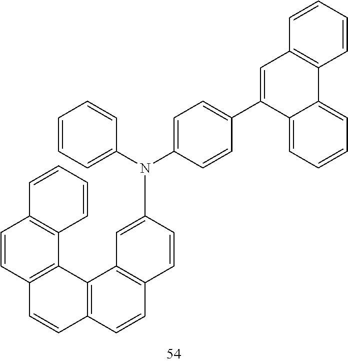 [SMILES: c1ccc(N(c2ccc(-c3cc4ccccc4c4ccccc34)cc2)c2ccc3ccc4ccc5ccc6ccccc6c5c4c3c2)cc1] | 622.3 | 67 |
| 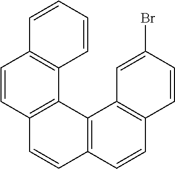 [SMILES: Brc1ccc2ccc3ccc4ccc5ccccc5c4c3c2c1] | 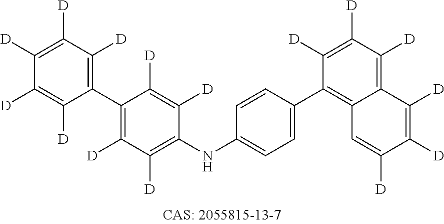 [SMILES: [2H]c1cc2c(-c3ccc(Nc4c([2H])c([2H])c(-c5c([2H])c([2H])c([2H])c([2H])c5[2H])c([2H])c4[2H])cc3)c([2H])c([2H])c([2H])c2c([2H])c1[2H]] | 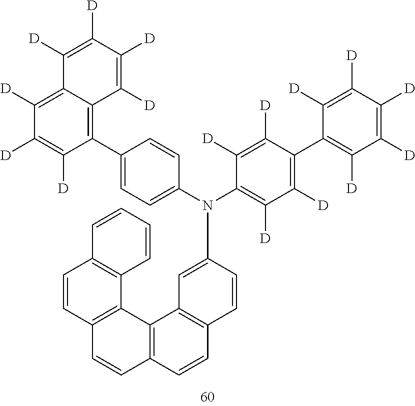 [SMILES: [2H]c1c([2H])c([2H])c(-c2c([2H])c([2H])c(N(c3ccc(-c4c([2H])c([2H])c([2H])c5c([2H])c([2H])c([2H])c([2H])c45)cc3)c3ccc4ccc5ccc6ccc7ccccc7c6c5c4c3)c([2H])c2[2H])c([2H])c1[2H]] | 664.4 | 66 |
| 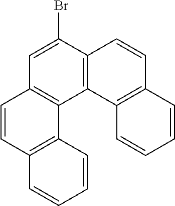 [SMILES: Brc1cc2ccc3ccccc3c2c2c1ccc1ccccc12] | 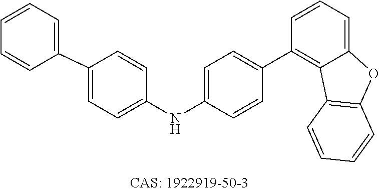 [SMILES: c1ccc(-c2ccc(Nc3ccc(-c4cccc5oc6ccccc6c45)cc3)cc2)cc1] | 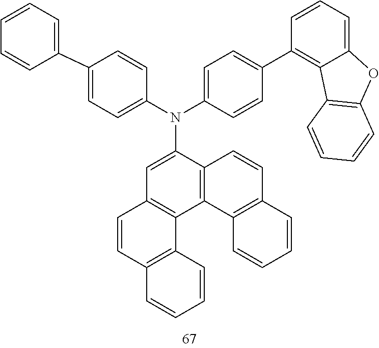 [SMILES: c1ccc(-c2ccc(N(c3ccc(-c4cccc5oc6ccccc6c45)cc3)c3cc4ccc5ccccc5c4c4c3ccc3ccccc34)cc2)cc1] | 688.3 | 68 |
| 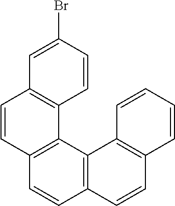 [SMILES: Brc1ccc2c(ccc3ccc4ccc5ccccc5c4c32)c1] | 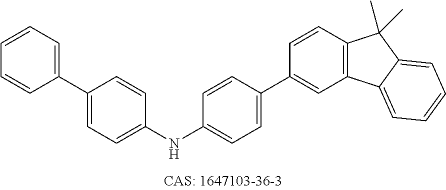 [SMILES: CC1(C)c2ccccc2-c2cc(-c3ccc(Nc4ccc(-c5ccccc5)cc4)cc3)ccc21] | 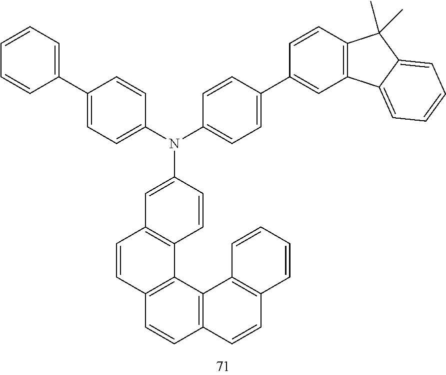 [SMILES: CC1(C)c2ccccc2-c2cc(-c3ccc(N(c4ccc(-c5ccccc5)cc4)c4ccc5c(ccc6ccc7ccc8ccccc8c7c65)c4)cc3)ccc21] | 714.3 | 70 |
| 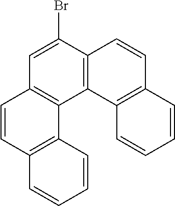 [SMILES: Brc1cc2ccc3ccccc3c2c2c1ccc1ccccc12] | 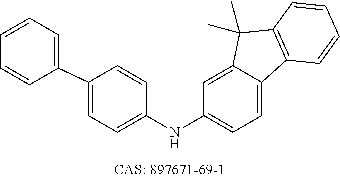 [SMILES: CC1(C)c2ccccc2-c2ccc(Nc3ccc(-c4ccccc4)cc3)cc21] | 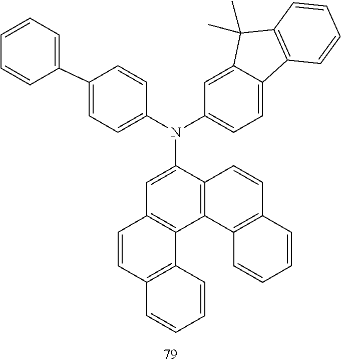 [SMILES: CC1(C)c2ccccc2-c2ccc(N(c3ccc(-c4ccccc4)cc3)c3cc4ccc5ccccc5c4c4c3ccc3ccccc34)cc21] | 638.3 | 75 |
| 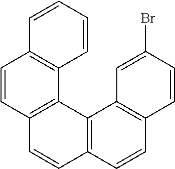 [SMILES: Brc1ccc2ccc3ccc4ccc5ccccc5c4c3c2c1] | 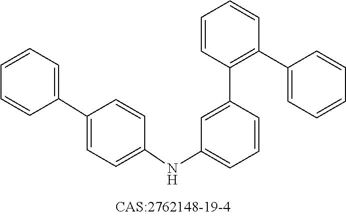 [SMILES: c1ccc(-c2ccc(Nc3cccc(-c4ccccc4-c4ccccc4)c3)cc2)cc1] | 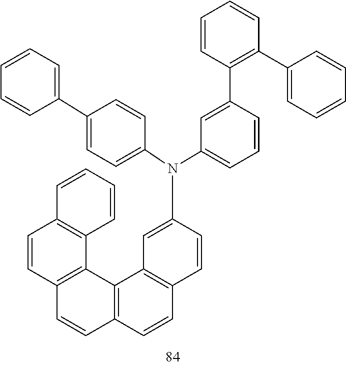 [SMILES: c1ccc(-c2ccc(N(c3cccc(-c4ccccc4-c4ccccc4)c3)c3ccc4ccc5ccc6ccc7ccccc7c6c5c4c3)cc2)cc1] | 674.3 | 71 |
| 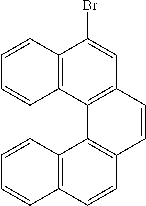 [SMILES: Brc1cc2ccc3ccc4ccccc4c3c2c2ccccc12] | 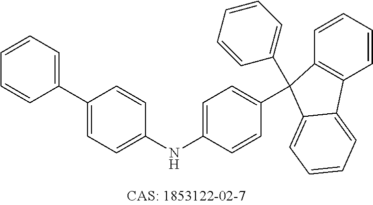 [SMILES: c1ccc(-c2ccc(Nc3ccc(C4(c5ccccc5)c5ccccc5-c5ccccc54)cc3)cc2)cc1] | 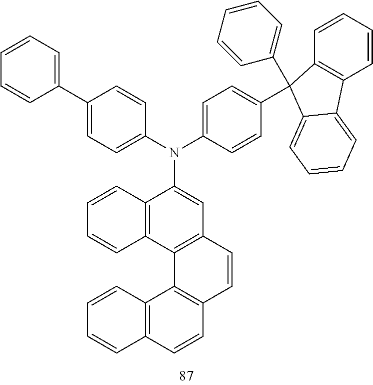 [SMILES: c1ccc(-c2ccc(N(c3ccc(C4(c5ccccc5)c5ccccc5-c5ccccc54)cc3)c3cc4ccc5ccc6ccccc6c5c4c4ccccc34)cc2)cc1] | 762.3 | 72 |
| 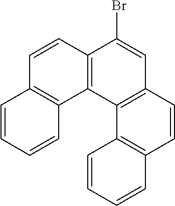 [SMILES: Brc1cc2ccc3ccccc3c2c2c1ccc1ccccc12] | 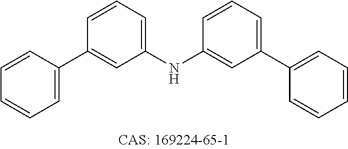 [SMILES: c1ccc(-c2cccc(Nc3cccc(-c4ccccc4)c3)c2)cc1] | 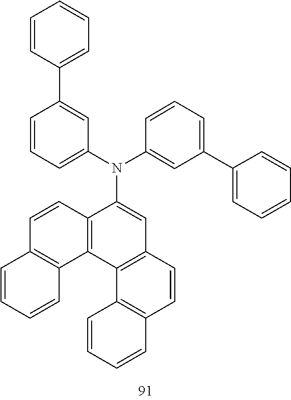 [SMILES: c1ccc(-c2cccc(N(c3cccc(-c4ccccc4)c3)c3cc4ccc5ccccc5c4c4c3ccc3ccccc34)c2)cc1] | 598.3 | 68 |
| 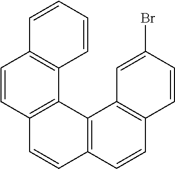 [SMILES: Brc1ccc2ccc3ccc4ccc5ccccc5c4c3c2c1] | 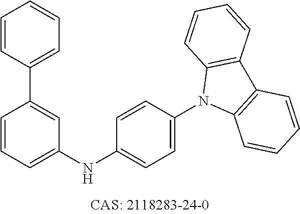 [SMILES: c1ccc(-c2cccc(Nc3ccc(-n4c5ccccc5c5ccccc54)cc3)c2)cc1] | 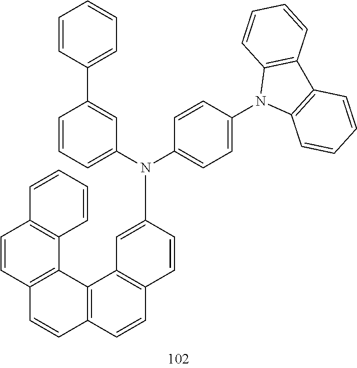 [SMILES: c1ccc(-c2cccc(N(c3ccc(-n4c5ccccc5c5ccccc54)cc3)c3ccc4ccc5ccc6ccc7ccccc7c6c5c4c3)c2)cc1] | 687.3 | 65 |
| 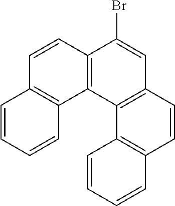 [SMILES: Brc1cc2ccc3ccccc3c2c2c1ccc1ccccc12] | 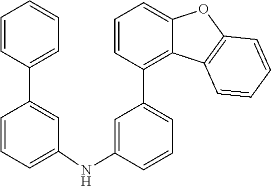 [SMILES: c1ccc(-c2cccc(Nc3cccc(-c4cccc5oc6ccccc6c45)c3)c2)cc1] | 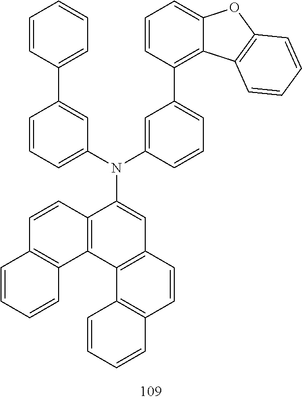 [SMILES: c1ccc(-c2cccc(N(c3cccc(-c4cccc5oc6ccccc6c45)c3)c3cc4ccc5ccccc5c4c4c3ccc3ccccc34)c2)cc1] | 688.3 | 72 |
| 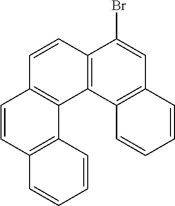 [SMILES: Brc1cc2ccccc2c2c1ccc1ccc3ccccc3c12] | 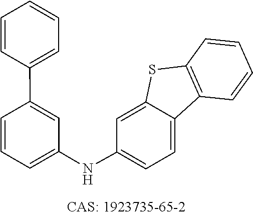 [SMILES: c1ccc(-c2cccc(Nc3ccc4c(c3)sc3ccccc34)c2)cc1] | 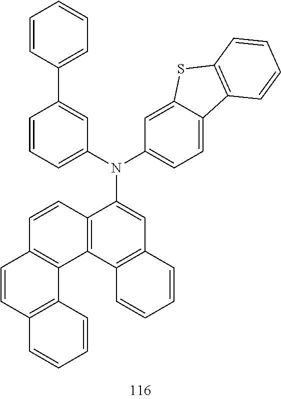 [SMILES: c1ccc(-c2cccc(N(c3ccc4c(c3)sc3ccccc34)c3cc4ccccc4c4c3ccc3ccc5ccccc5c34)c2)cc1] | 628.2 | 72 |
| 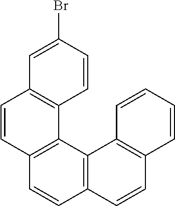 [SMILES: Brc1ccc2c(ccc3ccc4ccc5ccccc5c4c32)c1] | 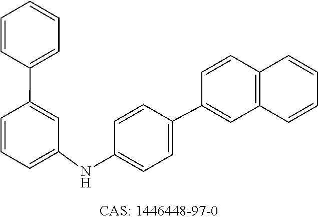 [SMILES: c1ccc(-c2cccc(Nc3ccc(-c4ccc5ccccc5c4)cc3)c2)cc1] | 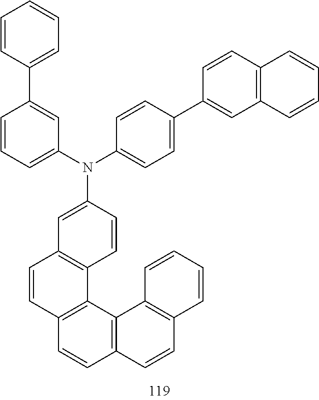 [SMILES: c1ccc(-c2cccc(N(c3ccc(-c4ccc5ccccc5c4)cc3)c3ccc4c(ccc5ccc6ccc7ccccc7c6c54)c3)c2)cc1] | 648.3 | 72 |
| 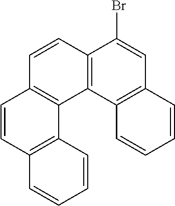 [SMILES: Brc1cc2ccccc2c2c1ccc1ccc3ccccc3c12] | 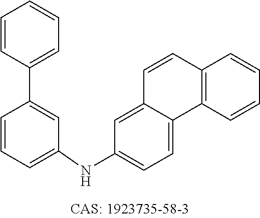 [SMILES: c1ccc(-c2cccc(Nc3ccc4c(ccc5ccccc54)c3)c2)cc1] | 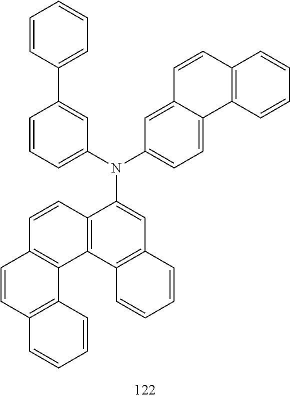 [SMILES: c1ccc(-c2cccc(N(c3ccc4c(ccc5ccccc54)c3)c3cc4ccccc4c4c3ccc3ccc5ccccc5c34)c2)cc1] | 622.3 | 69 |
| 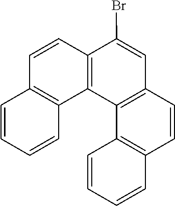 [SMILES: Brc1cc2ccc3ccccc3c2c2c1ccc1ccccc12] | 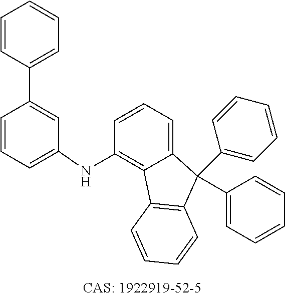 [SMILES: c1ccc(-c2cccc(Nc3cccc4c3-c3ccccc3C4(c3ccccc3)c3ccccc3)c2)cc1] | 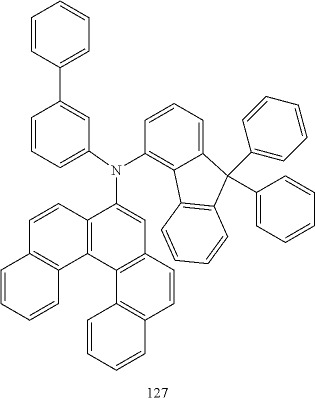 [SMILES: c1ccc(-c2cccc(N(c3cccc4c3-c3ccccc3C4(c3ccccc3)c3ccccc3)c3cc4ccc5ccccc5c4c4c3ccc3ccccc34)c2)cc1] | 762.3 | 62 |
| 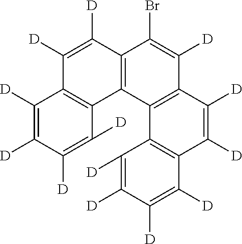 [SMILES: [2H]c1c([2H])c([2H])c2c(c1[2H])c([2H])c([2H])c1c([2H])c(Br)c3c([2H])c([2H])c4c([2H])c([2H])c([2H])c([2H])c4c3c12] | 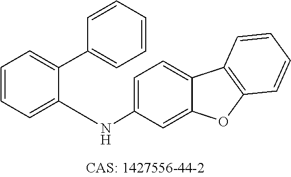 [SMILES: c1ccc(-c2ccccc2Nc2ccc3c(c2)oc2ccccc23)cc1] | 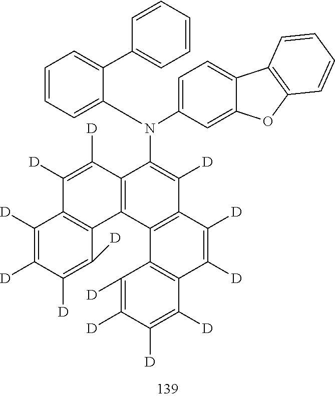 [SMILES: [2H]c1c([2H])c([2H])c2c(c1[2H])c([2H])c([2H])c1c([2H])c(N(c3ccc4c(c3)oc3ccccc34)c3ccccc3-c3ccccc3)c3c([2H])c([2H])c4c([2H])c([2H])c([2H])c([2H])c4c3c12] | 625.3 | 60 |
| 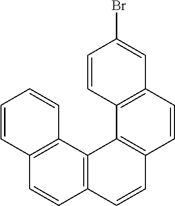 [SMILES: Brc1ccc2c(ccc3ccc4ccc5ccccc5c4c32)c1] | 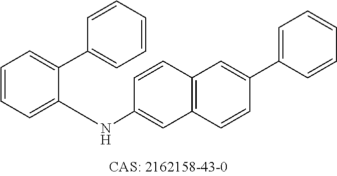 [SMILES: c1ccc(-c2ccc3cc(Nc4ccccc4-c4ccccc4)ccc3c2)cc1] | 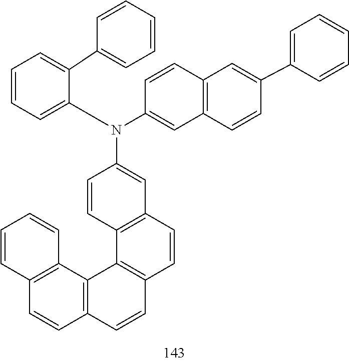 [SMILES: c1ccc(-c2ccc3cc(N(c4ccc5c(ccc6ccc7ccc8ccccc8c7c65)c4)c4ccccc4-c4ccccc4)ccc3c2)cc1] | 648.3 | 57 |
| 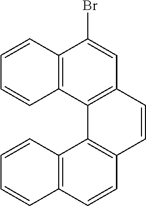 [SMILES: Brc1cc2ccc3ccc4ccccc4c3c2c2ccccc12] | 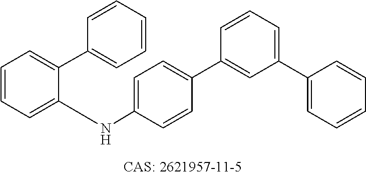 [SMILES: c1ccc(-c2cccc(-c3ccc(Nc4ccccc4-c4ccccc4)cc3)c2)cc1] | 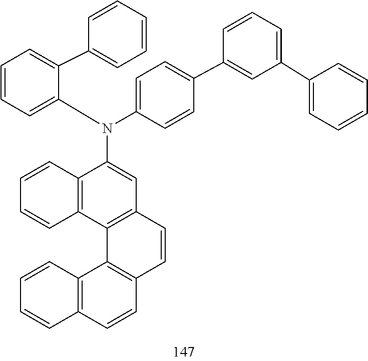 [SMILES: c1ccc(-c2cccc(-c3ccc(N(c4ccccc4-c4ccccc4)c4cc5ccc6ccc7ccccc7c6c5c5ccccc45)cc3)c2)cc1] | 674.3 | 59 |
| 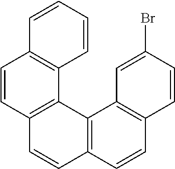 [SMILES: Brc1ccc2ccc3ccc4ccc5ccccc5c4c3c2c1] | 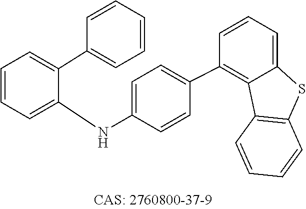 [SMILES: c1ccc(-c2ccccc2Nc2ccc(-c3cccc4sc5ccccc5c34)cc2)cc1] | 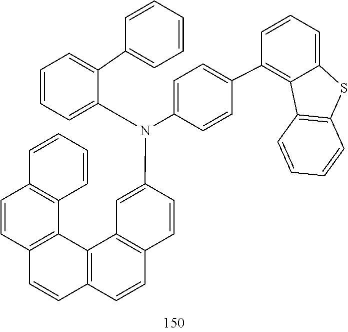 [SMILES: c1ccc(-c2ccccc2N(c2ccc(-c3cccc4sc5ccccc5c34)cc2)c2ccc3ccc4ccc5ccc6ccccc6c5c4c3c2)cc1] | 704.2 | 62 |
| 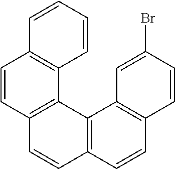 [SMILES: Brc1ccc2ccc3ccc4ccc5ccccc5c4c3c2c1] | 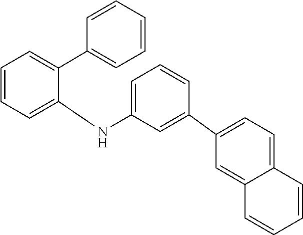 [SMILES: c1ccc(-c2ccccc2Nc2cccc(-c3ccc4ccccc4c3)c2)cc1] | 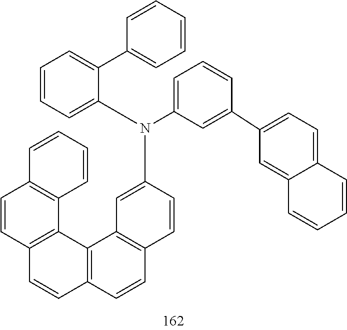 [SMILES: c1ccc(-c2ccccc2N(c2cccc(-c3ccc4ccccc4c3)c2)c2ccc3ccc4ccc5ccc6ccccc6c5c4c3c2)cc1] | 648.3 | 57 |
| 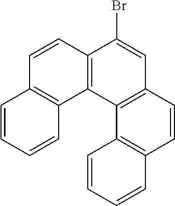 [SMILES: Brc1cc2ccc3ccccc3c2c2c1ccc1ccccc12] | 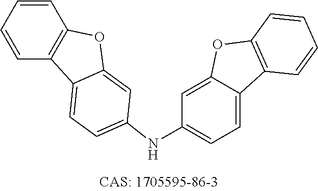 [SMILES: c1ccc2c(c1)oc1cc(Nc3ccc4c(c3)oc3ccccc34)ccc12] | 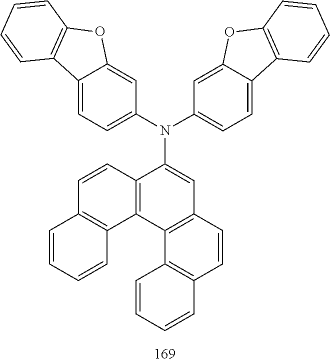 [SMILES: c1ccc2c(c1)ccc1cc(N(c3ccc4c(c3)oc3ccccc34)c3ccc4c(c3)oc3ccccc34)c3ccc4ccccc4c3c12] | 626.2 | 70 |
| 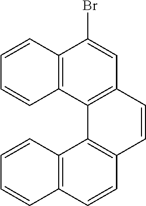 [SMILES: Brc1cc2ccc3ccc4ccccc4c3c2c2ccccc12] | 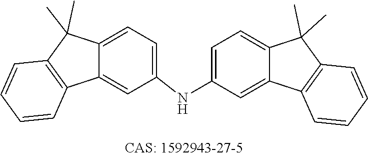 [SMILES: CC1(C)c2ccccc2-c2cc(Nc3ccc4c(c3)-c3ccccc3C4(C)C)ccc21] | 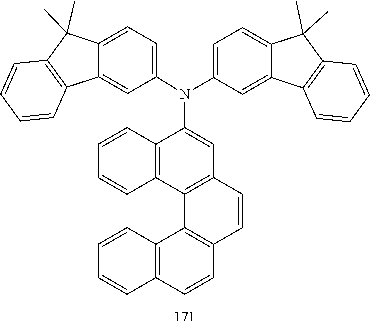 [SMILES: CC1(C)c2ccccc2-c2cc(N(c3ccc4c(c3)-c3ccccc3C4(C)C)c3cc4ccc5ccc6ccccc6c5c4c4ccccc34)ccc21] | 678.3 | 71 |
| 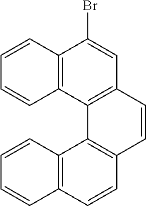 [SMILES: Brc1cc2ccc3ccc4ccccc4c3c2c2ccccc12] | 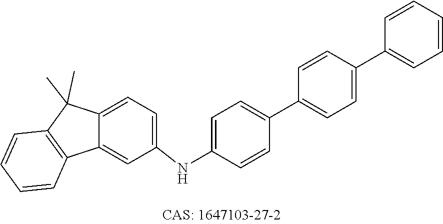 [SMILES: CC1(C)c2ccccc2-c2cc(Nc3ccc(-c4ccc(-c5ccccc5)cc4)cc3)ccc21] | 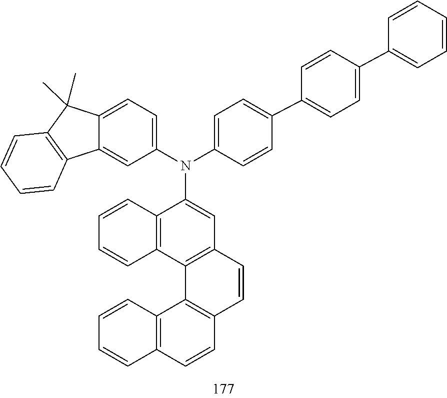 [SMILES: CC1(C)c2ccccc2-c2cc(N(c3ccc(-c4ccc(-c5ccccc5)cc4)cc3)c3cc4ccc5ccc6ccccc6c5c4c4ccccc34)ccc21] | 714.3 | 68 |
| 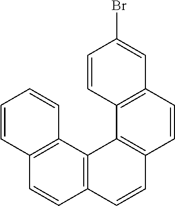 [SMILES: Brc1ccc2c(ccc3ccc4ccc5ccccc5c4c32)c1] | 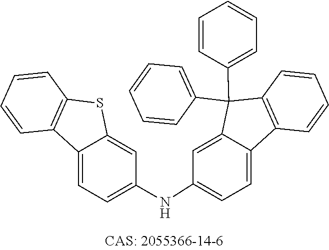 [SMILES: c1ccc(C2(c3ccccc3)c3ccccc3-c3ccc(Nc4ccc5c(c4)sc4ccccc45)cc32)cc1] | 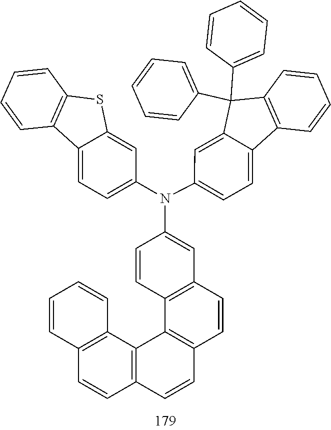 [SMILES: c1ccc(C2(c3ccccc3)c3ccccc3-c3ccc(N(c4ccc5c(ccc6ccc7ccc8ccccc8c7c65)c4)c4ccc5c(c4)sc4ccccc45)cc32)cc1] | 792.3 | 65 |
| 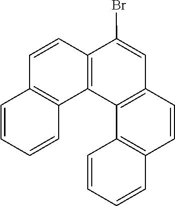 [SMILES: Brc1cc2ccc3ccccc3c2c2c1ccc1ccccc12] | 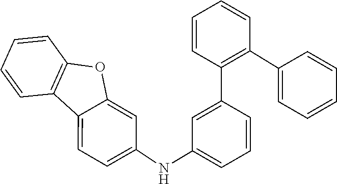 [SMILES: c1ccc(-c2ccccc2-c2cccc(Nc3ccc4c(c3)oc3ccccc34)c2)cc1] | 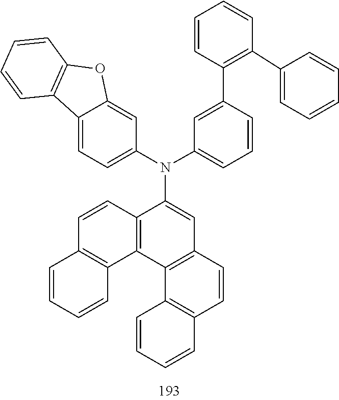 [SMILES: c1ccc(-c2ccccc2-c2cccc(N(c3ccc4c(c3)oc3ccccc34)c3cc4ccc5ccccc5c4c4c3ccc3ccccc34)c2)cc1] | 688.3 | 65 |
| 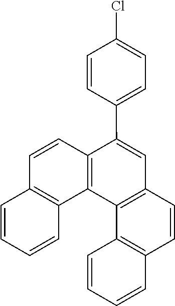 [SMILES: Clc1ccc(-c2cc3ccc4ccccc4c3c3c2ccc2ccccc23)cc1] | 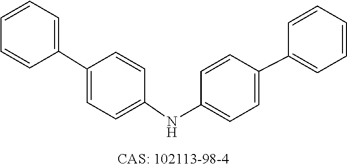 [SMILES: c1ccc(-c2ccc(Nc3ccc(-c4ccccc4)cc3)cc2)cc1] | 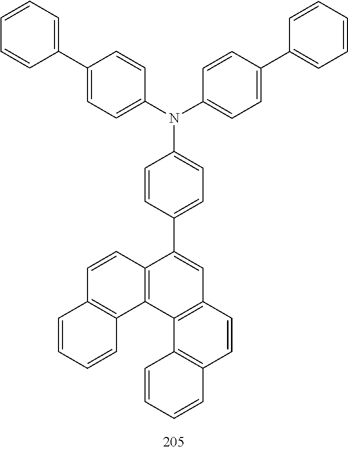 [SMILES: c1ccc(-c2ccc(N(c3ccc(-c4ccccc4)cc3)c3ccc(-c4cc5ccc6ccccc6c5c5c4ccc4ccccc45)cc3)cc2)cc1] | 674.3 | 73 |
| 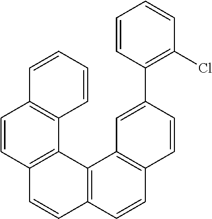 [SMILES: Clc1ccccc1-c1ccc2ccc3ccc4ccc5ccccc5c4c3c2c1] | 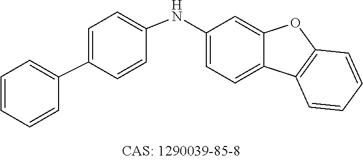 [SMILES: c1ccc(-c2ccc(Nc3ccc4c(c3)oc3ccccc34)cc2)cc1] | 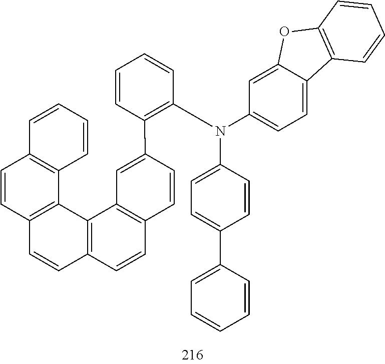 [SMILES: c1ccc(-c2ccc(N(c3ccc4c(c3)oc3ccccc34)c3ccccc3-c3ccc4ccc5ccc6ccc7ccccc7c6c5c4c3)cc2)cc1] | 688.3 | 58 |
| 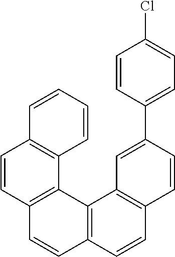 [SMILES: Clc1ccc(-c2ccc3ccc4ccc5ccc6ccccc6c5c4c3c2)cc1] | 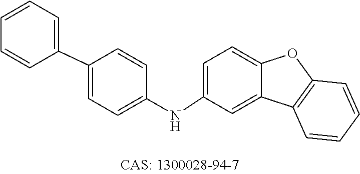 [SMILES: c1ccc(-c2ccc(Nc3ccc4oc5ccccc5c4c3)cc2)cc1] | 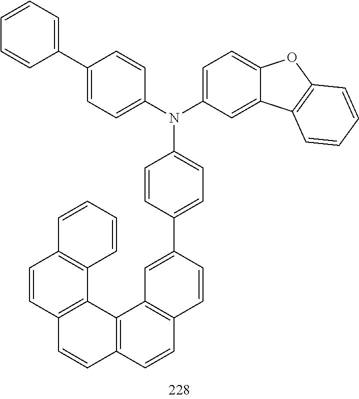 [SMILES: c1ccc(-c2ccc(N(c3ccc(-c4ccc5ccc6ccc7ccc8ccccc8c7c6c5c4)cc3)c3ccc4oc5ccccc5c4c3)cc2)cc1] | 688.3 | 67 |
| 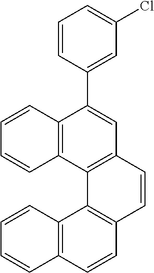 [SMILES: Clc1cccc(-c2cc3ccc4ccc5ccccc5c4c3c3ccccc23)c1] | 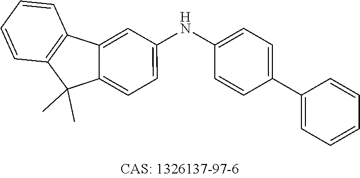 [SMILES: CC1(C)c2ccccc2-c2cc(Nc3ccc(-c4ccccc4)cc3)ccc21] | 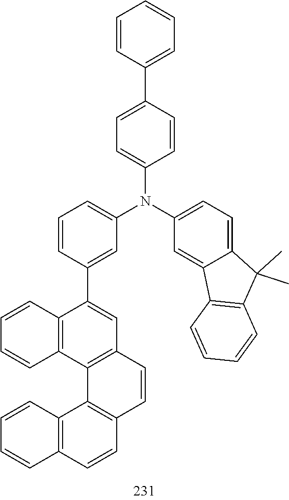 [SMILES: CC1(C)c2ccccc2-c2cc(N(c3ccc(-c4ccccc4)cc3)c3cccc(-c4cc5ccc6ccc7ccccc7c6c5c5ccccc45)c3)ccc21] | 714.3 | 75 |
| 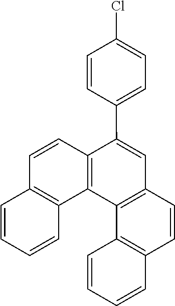 [SMILES: Clc1ccc(-c2cc3ccc4ccccc4c3c3c2ccc2ccccc23)cc1] | 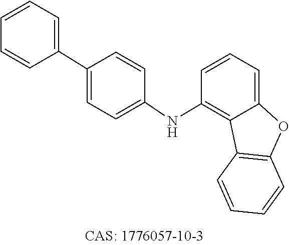 [SMILES: c1ccc(-c2ccc(Nc3cccc4oc5ccccc5c34)cc2)cc1] | 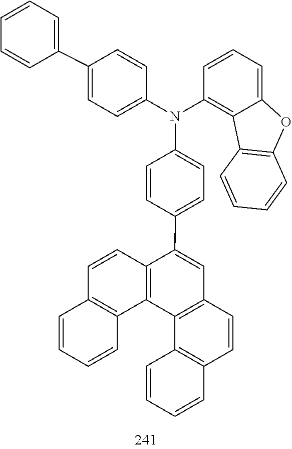 [SMILES: c1ccc(-c2ccc(N(c3ccc(-c4cc5ccc6ccccc6c5c5c4ccc4ccccc45)cc3)c3cccc4oc5ccccc5c34)cc2)cc1] | 688.3 | 69 |
| 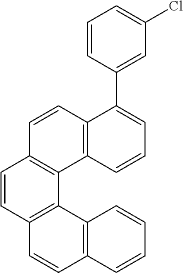 [SMILES: Clc1cccc(-c2cccc3c2ccc2ccc4ccc5ccccc5c4c23)c1] | 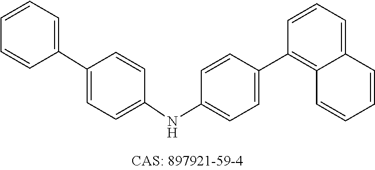 [SMILES: c1ccc(-c2ccc(Nc3ccc(-c4cccc5ccccc45)cc3)cc2)cc1] | 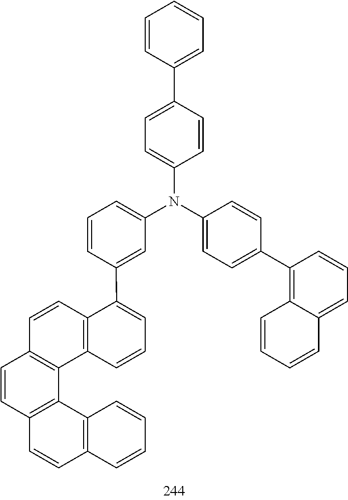 [SMILES: c1ccc(-c2ccc(N(c3ccc(-c4cccc5ccccc45)cc3)c3cccc(-c4cccc5c4ccc4ccc6ccc7ccccc7c6c45)c3)cc2)cc1] | 724.3 | 66 |
| 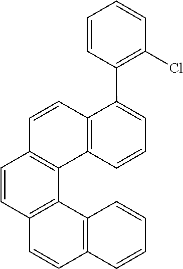 [SMILES: Clc1ccccc1-c1cccc2c1ccc1ccc3ccc4ccccc4c3c12] | 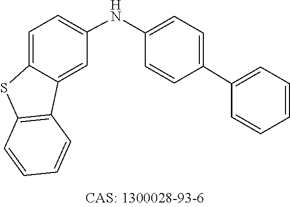 [SMILES: c1ccc(-c2ccc(Nc3ccc4sc5ccccc5c4c3)cc2)cc1] | 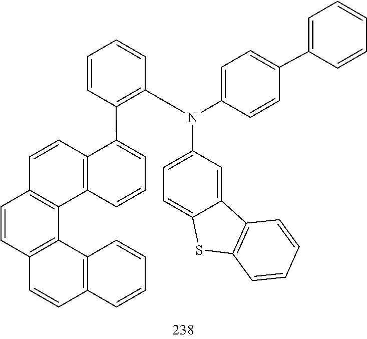 [SMILES: c1ccc(-c2ccc(N(c3ccc4sc5ccccc5c4c3)c3ccccc3-c3cccc4c3ccc3ccc5ccc6ccccc6c5c34)cc2)cc1] | 704.2 | 57 |
| 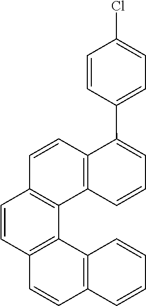 [SMILES: Clc1ccc(-c2cccc3c2ccc2ccc4ccc5ccccc5c4c23)cc1] | 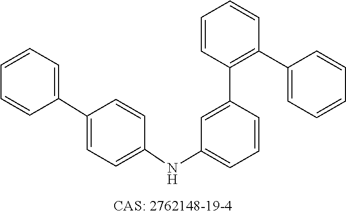 [SMILES: c1ccc(-c2ccc(Nc3cccc(-c4ccccc4-c4ccccc4)c3)cc2)cc1] | 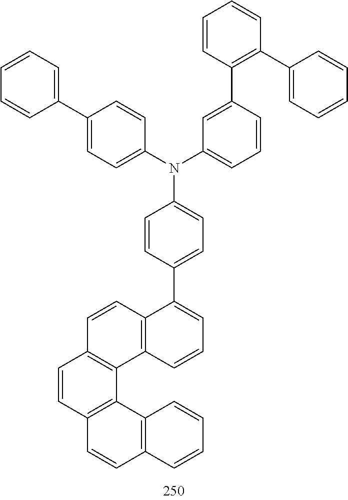 [SMILES: c1ccc(-c2ccc(N(c3ccc(-c4cccc5c4ccc4ccc6ccc7ccccc7c6c45)cc3)c3cccc(-c4ccccc4-c4ccccc4)c3)cc2)cc1] | 750.3 | 66 |
| 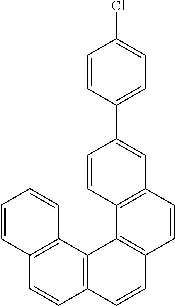 [SMILES: Clc1ccc(-c2ccc3c(ccc4ccc5ccc6ccccc6c5c43)c2)cc1] | 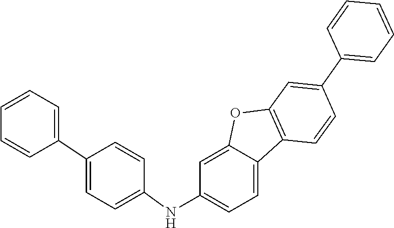 [SMILES: c1ccc(-c2ccc(Nc3ccc4c(c3)oc3cc(-c5ccccc5)ccc34)cc2)cc1] | 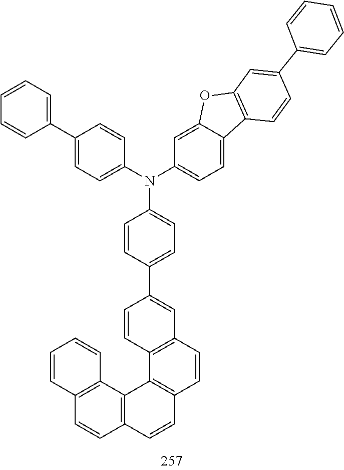 [SMILES: c1ccc(-c2ccc(N(c3ccc(-c4ccc5c(ccc6ccc7ccc8ccccc8c7c65)c4)cc3)c3ccc4c(c3)oc3cc(-c5ccccc5)ccc34)cc2)cc1] | 764.3 | 70 |
| 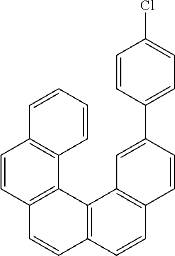 [SMILES: Clc1ccc(-c2ccc3ccc4ccc5ccc6ccccc6c5c4c3c2)cc1] | 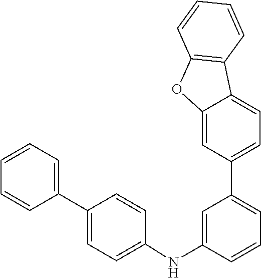 [SMILES: c1ccc(-c2ccc(Nc3cccc(-c4ccc5c(c4)oc4ccccc45)c3)cc2)cc1] | 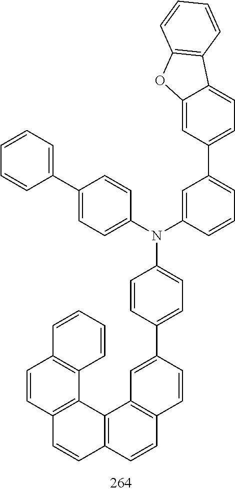 [SMILES: c1ccc(-c2ccc(N(c3ccc(-c4ccc5ccc6ccc7ccc8ccccc8c7c6c5c4)cc3)c3cccc(-c4ccc5c(c4)oc4ccccc45)c3)cc2)cc1] | 764.3 | 69 |
| 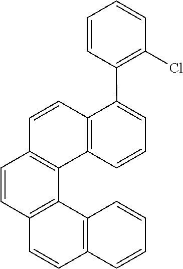 [SMILES: Clc1ccccc1-c1cccc2c1ccc1ccc3ccc4ccccc4c3c12] | 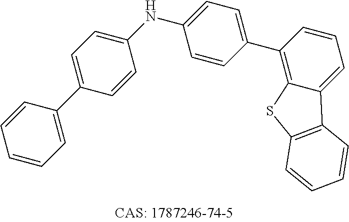 [SMILES: c1ccc(-c2ccc(Nc3ccc(-c4cccc5c4sc4ccccc45)cc3)cc2)cc1] | 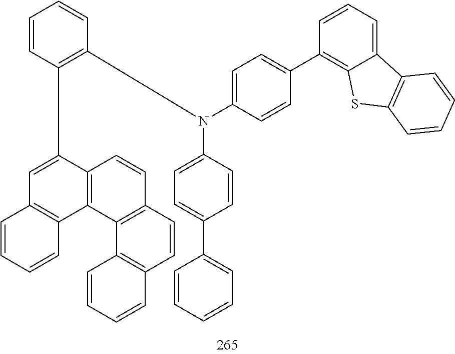 [SMILES: c1ccc(-c2ccc(N(c3ccc(-c4cccc5c4sc4ccccc45)cc3)c3ccccc3-c3cc4ccccc4c4c3ccc3ccc5ccccc5c34)cc2)cc1] | 780.3 | 52 |
| 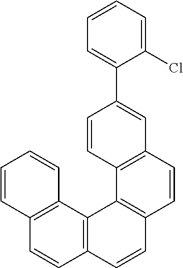 [SMILES: Clc1ccccc1-c1ccc2c(ccc3ccc4ccc5ccccc5c4c32)c1] | 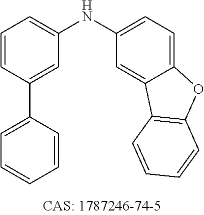 [SMILES: c1ccc(-c2cccc(Nc3ccc4oc5ccccc5c4c3)c2)cc1] | 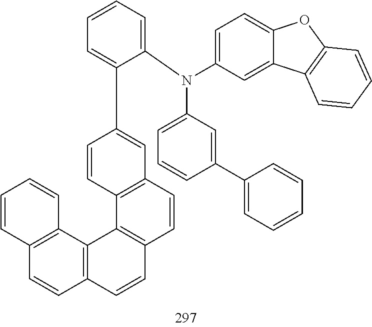 [SMILES: c1ccc(-c2cccc(N(c3ccc4oc5ccccc5c4c3)c3ccccc3-c3ccc4c(ccc5ccc6ccc7ccccc7c6c54)c3)c2)cc1] | 688.3 | 56 |
| 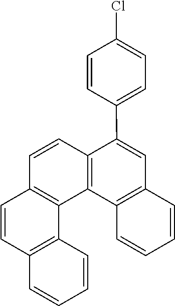 [SMILES: Clc1ccc(-c2cc3ccccc3c3c2ccc2ccc4ccccc4c23)cc1] | 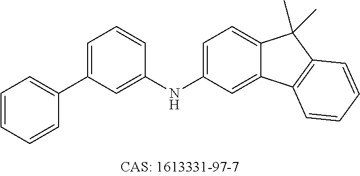 [SMILES: CC1(C)c2ccccc2-c2cc(Nc3cccc(-c4ccccc4)c3)ccc21] | 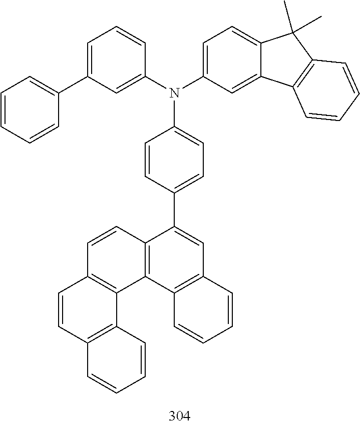 [SMILES: CC1(C)c2ccccc2-c2cc(N(c3ccc(-c4cc5ccccc5c5c4ccc4ccc6ccccc6c45)cc3)c3cccc(-c4ccccc4)c3)ccc21] | 714.3 | 69 |
| 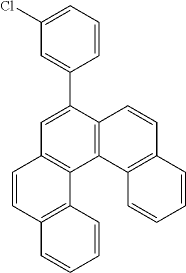 [SMILES: Clc1cccc(-c2cc3ccc4ccccc4c3c3c2ccc2ccccc23)c1] | 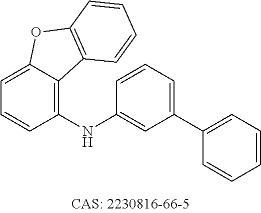 [SMILES: c1ccc(-c2cccc(Nc3cccc4oc5ccccc5c34)c2)cc1] | 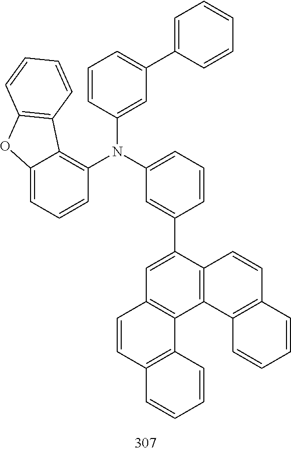 [SMILES: c1ccc(-c2cccc(N(c3cccc(-c4cc5ccc6ccccc6c5c5c4ccc4ccccc45)c3)c3cccc4oc5ccccc5c34)c2)cc1] | 688.3 | 66 |
| 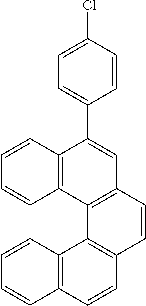 [SMILES: Clc1ccc(-c2cc3ccc4ccc5ccccc5c4c3c3ccccc23)cc1] | 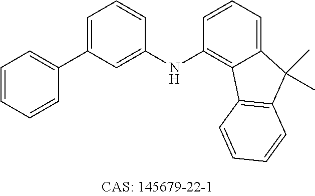 [SMILES: CC1(C)c2ccccc2-c2c(Nc3cccc(-c4ccccc4)c3)cccc21] | 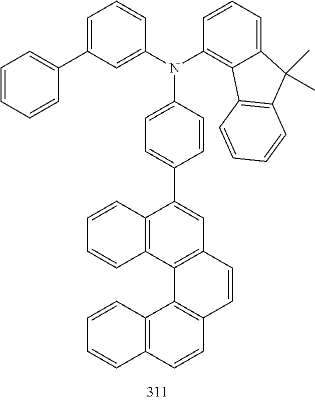 [SMILES: CC1(C)c2ccccc2-c2c(N(c3ccc(-c4cc5ccc6ccc7ccccc7c6c5c5ccccc45)cc3)c3cccc(-c4ccccc4)c3)cccc21] | 714.3 | 75 |
| 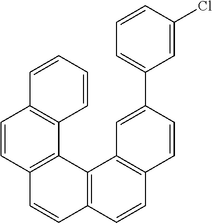 [SMILES: Clc1cccc(-c2ccc3ccc4ccc5ccc6ccccc6c5c4c3c2)c1] | 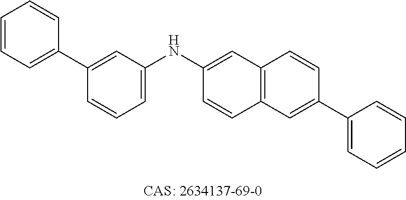 [SMILES: c1ccc(-c2cccc(Nc3ccc4cc(-c5ccccc5)ccc4c3)c2)cc1] | 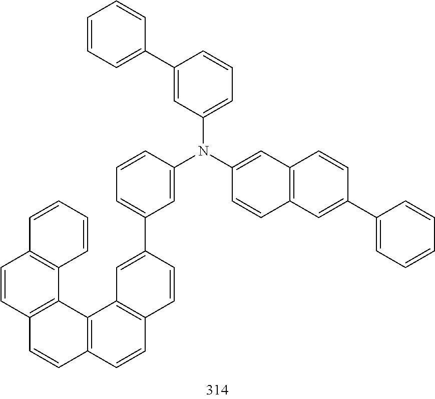 [SMILES: c1ccc(-c2cccc(N(c3cccc(-c4ccc5ccc6ccc7ccc8ccccc8c7c6c5c4)c3)c3ccc4cc(-c5ccccc5)ccc4c3)c2)cc1] | 724.3 | 70 |
| 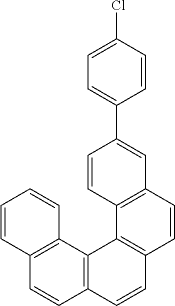 [SMILES: Clc1ccc(-c2ccc3c(ccc4ccc5ccc6ccccc6c5c43)c2)cc1] | 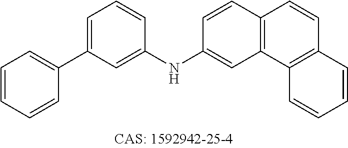 [SMILES: c1ccc(-c2cccc(Nc3ccc4ccc5ccccc5c4c3)c2)cc1] | 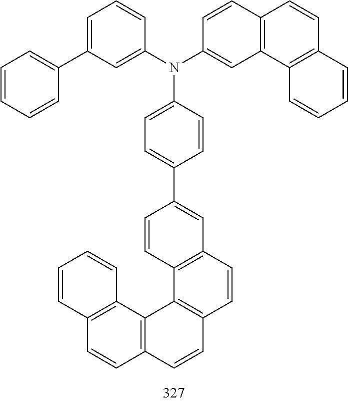 [SMILES: c1ccc(-c2cccc(N(c3ccc(-c4ccc5c(ccc6ccc7ccc8ccccc8c7c65)c4)cc3)c3ccc4ccc5ccccc5c4c3)c2)cc1] | 698.3 | 74 |
| 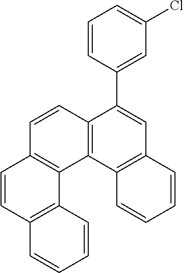 [SMILES: Clc1cccc(-c2cc3ccccc3c3c2ccc2ccc4ccccc4c23)c1] | 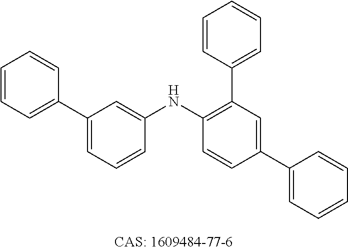 [SMILES: c1ccc(-c2cccc(Nc3ccc(-c4ccccc4)cc3-c3ccccc3)c2)cc1] | 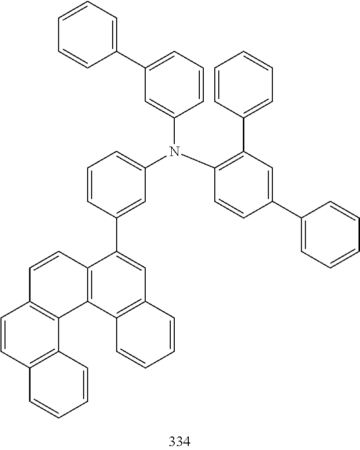 [SMILES: c1ccc(-c2cccc(N(c3cccc(-c4cc5ccccc5c5c4ccc4ccc6ccccc6c45)c3)c3ccc(-c4ccccc4)cc3-c3ccccc3)c2)cc1] | 750.3 | 58 |
| 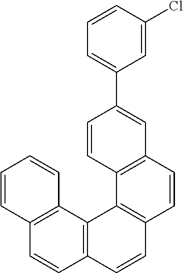 [SMILES: Clc1cccc(-c2ccc3c(ccc4ccc5ccc6ccccc6c5c43)c2)c1] | 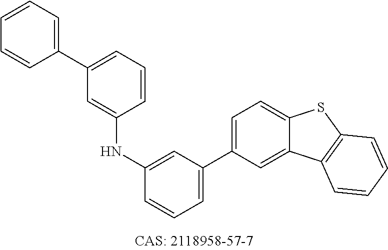 [SMILES: c1ccc(-c2cccc(Nc3cccc(-c4ccc5sc6ccccc6c5c4)c3)c2)cc1] | 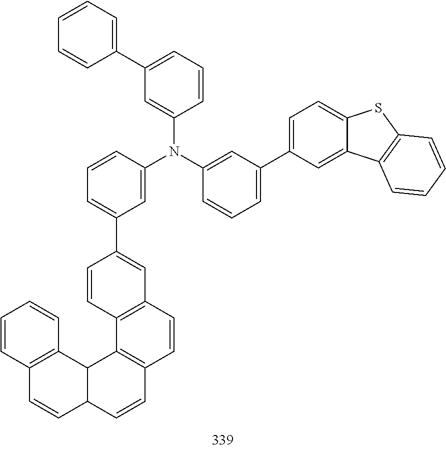 [SMILES: C1=CC2C=Cc3ccc4cc(-c5cccc(N(c6cccc(-c7ccccc7)c6)c6cccc(-c7ccc8sc9ccccc9c8c7)c6)c5)ccc4c3C2c2ccccc21] | 780.3 | 66 |
| 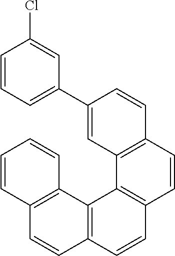 [SMILES: Clc1cccc(-c2ccc3ccc4ccc5ccc6ccccc6c5c4c3c2)c1] | 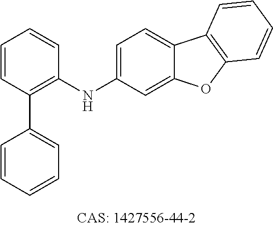 [SMILES: c1ccc(-c2ccccc2Nc2ccc3c(c2)oc2ccccc23)cc1] | 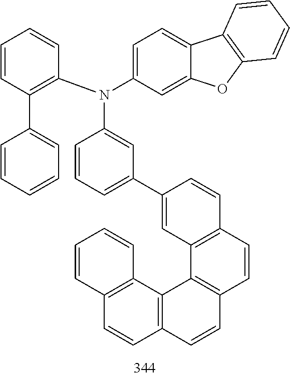 [SMILES: c1ccc(-c2ccccc2N(c2cccc(-c3ccc4ccc5ccc6ccc7ccccc7c6c5c4c3)c2)c2ccc3c(c2)oc2ccccc23)cc1] | 688.3 | 56 |
| 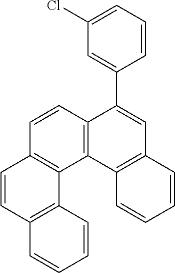 [SMILES: Clc1cccc(-c2cc3ccccc3c3c2ccc2ccc4ccccc4c23)c1] | 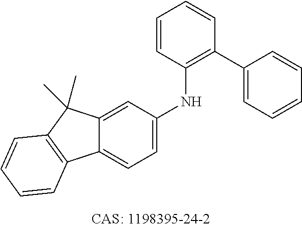 [SMILES: CC1(C)c2ccccc2-c2ccc(Nc3ccccc3-c3ccccc3)cc21] | 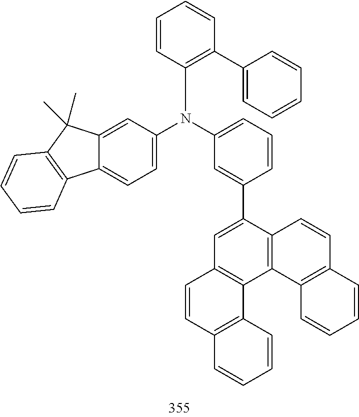 [SMILES: CC1(C)c2ccccc2-c2ccc(N(c3cccc(-c4cc5ccc6ccccc6c5c5c4ccc4ccccc45)c3)c3ccccc3-c3ccccc3)cc21] | 714.3 | 57 |
| 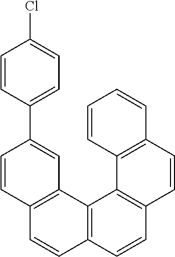 [SMILES: Clc1ccc(-c2ccc3ccc4ccc5ccc6ccccc6c5c4c3c2)cc1] | 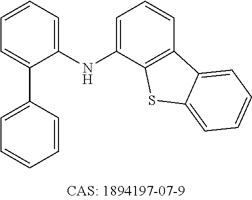 [SMILES: c1ccc(-c2ccccc2Nc2cccc3c2sc2ccccc23)cc1] | 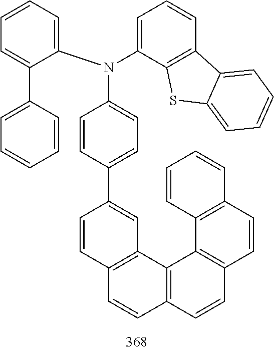 [SMILES: c1ccc(-c2ccccc2N(c2ccc(-c3ccc4ccc5ccc6ccc7ccccc7c6c5c4c3)cc2)c2cccc3c2sc2ccccc23)cc1] | 704.2 | 51 |
| 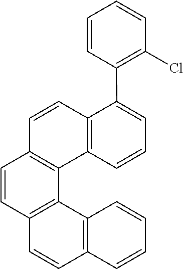 [SMILES: Clc1ccccc1-c1cccc2c1ccc1ccc3ccc4ccccc4c3c12] | 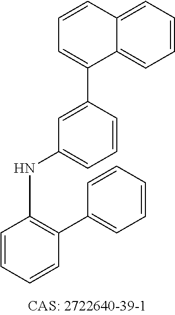 [SMILES: c1ccc(-c2ccccc2Nc2cccc(-c3cccc4ccccc34)c2)cc1] | 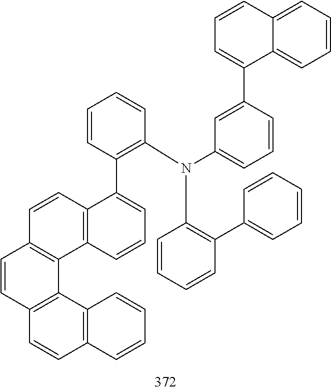 [SMILES: c1ccc(-c2ccccc2N(c2cccc(-c3cccc4ccccc34)c2)c2ccccc2-c2cccc3c2ccc2ccc4ccc5ccccc5c4c23)cc1] | 724.3 | 56 |
| 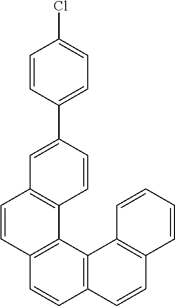 [SMILES: Clc1ccc(-c2ccc3c(ccc4ccc5ccc6ccccc6c5c43)c2)cc1] | 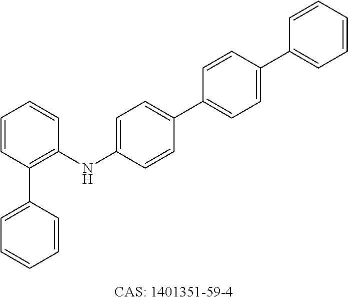 [SMILES: c1ccc(-c2ccc(-c3ccc(Nc4ccccc4-c4ccccc4)cc3)cc2)cc1] | 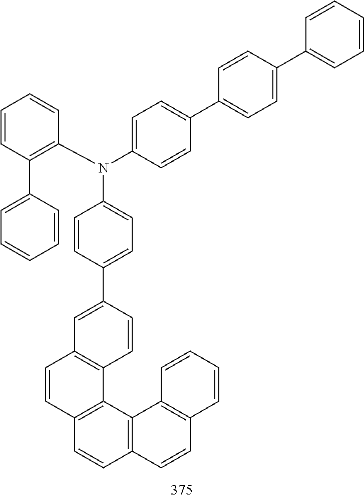 [SMILES: c1ccc(-c2ccc(-c3ccc(N(c4ccc(-c5ccc6c(ccc7ccc8ccc9ccccc9c8c76)c5)cc4)c4ccccc4-c4ccccc4)cc3)cc2)cc1] | 750.3 | 55 |
| 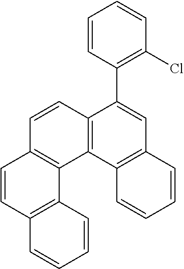 [SMILES: Clc1ccccc1-c1cc2ccccc2c2c1ccc1ccc3ccccc3c12] | 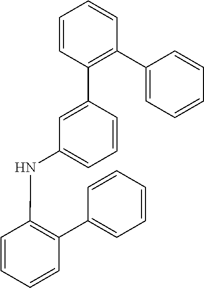 [SMILES: c1ccc(-c2ccccc2Nc2cccc(-c3ccccc3-c3ccccc3)c2)cc1] | 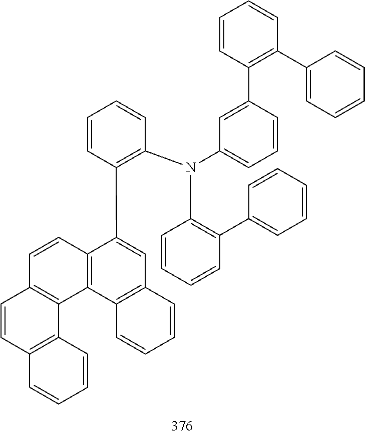 [SMILES: c1ccc(-c2ccccc2-c2cccc(N(c3ccccc3-c3ccccc3)c3ccccc3-c3cc4ccccc4c4c3ccc3ccc5ccccc5c34)c2)cc1] | 750.3 | 54 |
| 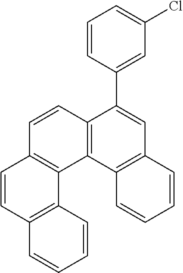 [SMILES: Clc1cccc(-c2cc3ccccc3c3c2ccc2ccc4ccccc4c23)c1] | 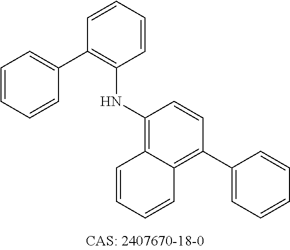 [SMILES: c1ccc(-c2ccccc2Nc2ccc(-c3ccccc3)c3ccccc23)cc1] | 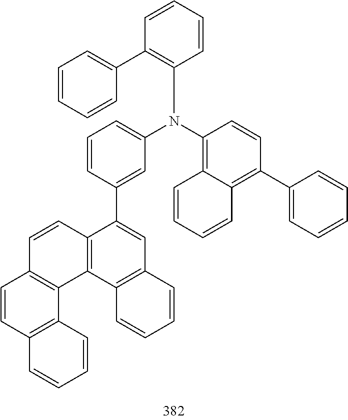 [SMILES: c1ccc(-c2ccccc2N(c2cccc(-c3cc4ccccc4c4c3ccc3ccc5ccccc5c34)c2)c2ccc(-c3ccccc3)c3ccccc23)cc1] | 724.3 | 52 |
| 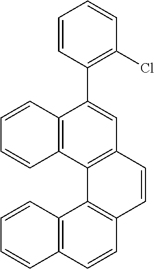 [SMILES: Clc1ccccc1-c1cc2ccc3ccc4ccccc4c3c2c2ccccc12] | 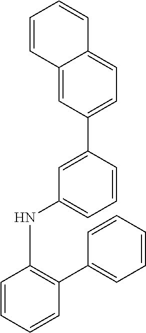 [SMILES: c1ccc(-c2ccccc2Nc2cccc(-c3ccc4ccccc4c3)c2)cc1] | 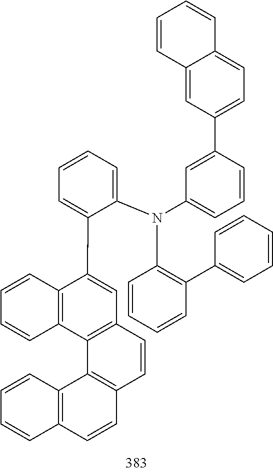 [SMILES: c1ccc(-c2ccccc2N(c2cccc(-c3ccc4ccccc4c3)c2)c2ccccc2-c2cc3ccc4ccc5ccccc5c4c3c3ccccc23)cc1] | 724.3 | 56 |
| 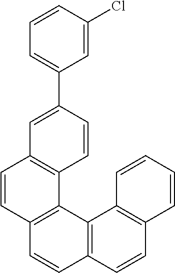 [SMILES: Clc1cccc(-c2ccc3c(ccc4ccc5ccc6ccccc6c5c43)c2)c1] | 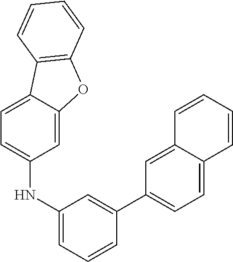 [SMILES: c1cc(Nc2ccc3c(c2)oc2ccccc23)cc(-c2ccc3ccccc3c2)c1] | 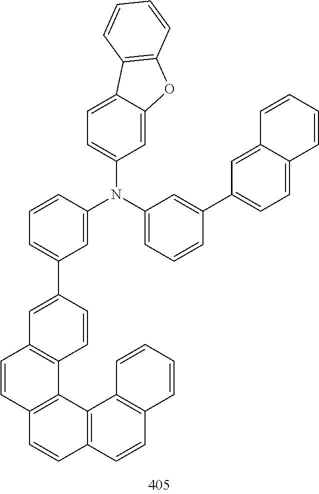 [SMILES: c1cc(-c2ccc3ccccc3c2)cc(N(c2cccc(-c3ccc4c(ccc5ccc6ccc7ccccc7c6c54)c3)c2)c2ccc3c(c2)oc2ccccc23)c1] | 738.3 | 67 |
| 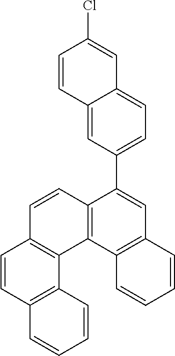 [SMILES: Clc1ccc2cc(-c3cc4ccccc4c4c3ccc3ccc5ccccc5c34)ccc2c1] | 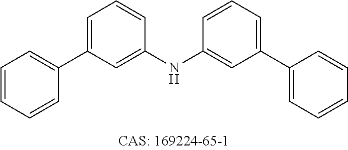 [SMILES: c1ccc(-c2cccc(Nc3cccc(-c4ccccc4)c3)c2)cc1] | 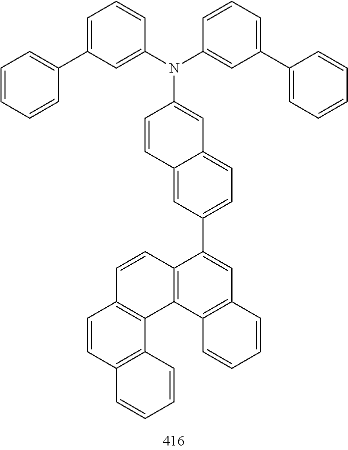 [SMILES: c1ccc(-c2cccc(N(c3cccc(-c4ccccc4)c3)c3ccc4cc(-c5cc6ccccc6c6c5ccc5ccc7ccccc7c56)ccc4c3)c2)cc1] | 724.3 | 70 |
| 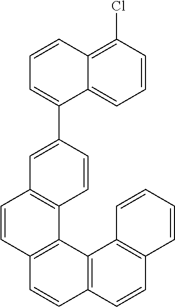 [SMILES: Clc1cccc2c(-c3ccc4c(ccc5ccc6ccc7ccccc7c6c54)c3)cccc12] | 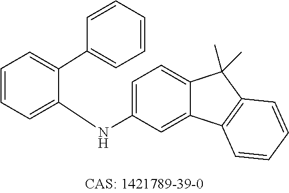 [SMILES: CC1(C)c2ccccc2-c2cc(Nc3ccccc3-c3ccccc3)ccc21] | 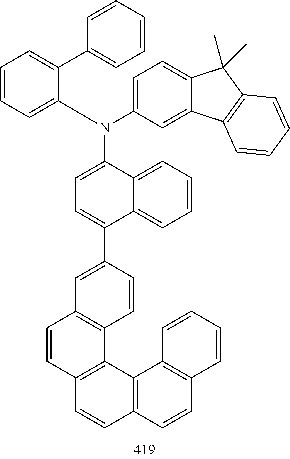 [SMILES: CC1(C)c2ccccc2-c2cc(N(c3ccccc3-c3ccccc3)c3ccc(-c4ccc5c(ccc6ccc7ccc8ccccc8c7c65)c4)c4ccccc34)ccc21] | 764.3 | 52 |
| 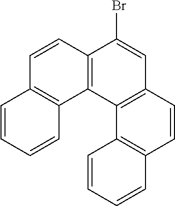 [SMILES: Brc1cc2ccc3ccccc3c2c2c1ccc1ccccc12] | 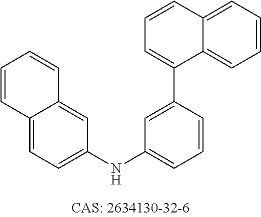 [SMILES: c1cc(Nc2ccc3ccccc3c2)cc(-c2cccc3ccccc23)c1] | 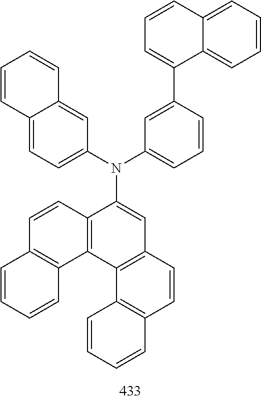 [SMILES: c1cc(-c2cccc3ccccc23)cc(N(c2ccc3ccccc3c2)c2cc3ccc4ccccc4c3c3c2ccc2ccccc23)c1] | 622.3 | 75 |
| 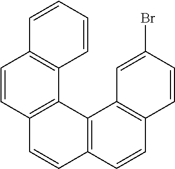 [SMILES: Brc1ccc2ccc3ccc4ccc5ccccc5c4c3c2c1] | 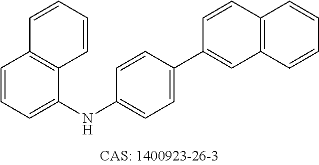 [SMILES: c1ccc2cc(-c3ccc(Nc4cccc5ccccc45)cc3)ccc2c1] | 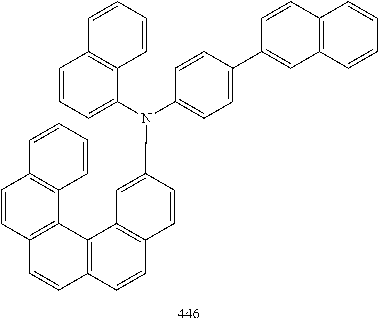 [SMILES: c1ccc2cc(-c3ccc(N(c4ccc5ccc6ccc7ccc8ccccc8c7c6c5c4)c4cccc5ccccc45)cc3)ccc2c1] | 622.3 | 69 |
| 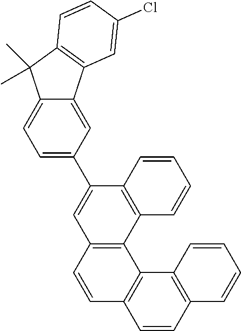 [SMILES: CC1(C)c2ccc(Cl)cc2-c2cc(-c3cc4ccc5ccc6ccccc6c5c4c4ccccc34)ccc21] | 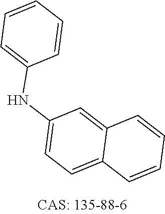 [SMILES: c1ccc(Nc2ccc3ccccc3c2)cc1] | 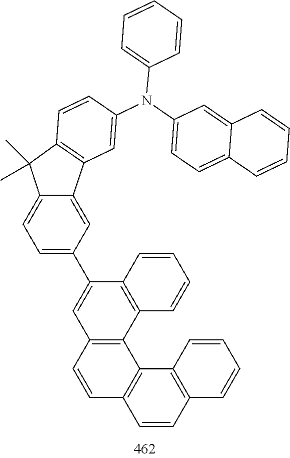 [SMILES: CC1(C)c2ccc(-c3cc4ccc5ccc6ccccc6c5c4c4ccccc34)cc2-c2cc(N(c3ccccc3)c3ccc4ccccc4c3)ccc21] | 688.3 | 74 |
| 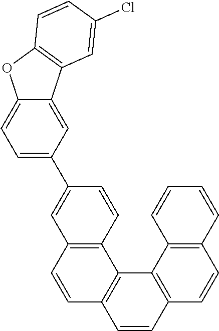 [SMILES: Clc1ccc2oc3ccc(-c4ccc5c(ccc6ccc7ccc8ccccc8c7c65)c4)cc3c2c1] | 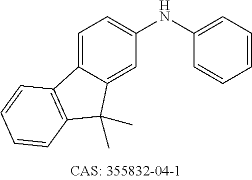 [SMILES: CC1(C)c2ccccc2-c2ccc(Nc3ccccc3)cc21] | 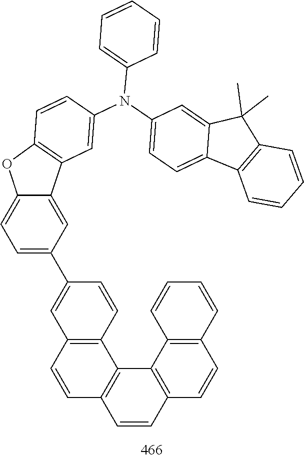 [SMILES: CC1(C)c2ccccc2-c2ccc(N(c3ccccc3)c3ccc4oc5ccc(-c6ccc7c(ccc8ccc9ccc%10ccccc%10c9c87)c6)cc5c4c3)cc21] | 728.3 | 69 |
| 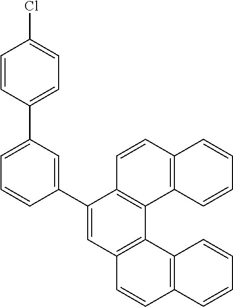 [SMILES: Clc1ccc(-c2cccc(-c3cc4ccc5ccccc5c4c4c3ccc3ccccc34)c2)cc1] | 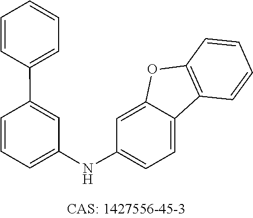 [SMILES: c1ccc(-c2cccc(Nc3ccc4c(c3)oc3ccccc34)c2)cc1] | 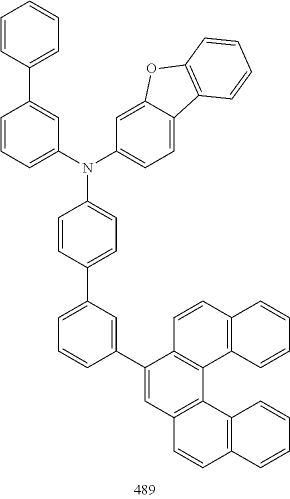 [SMILES: c1ccc(-c2cccc(N(c3ccc(-c4cccc(-c5cc6ccc7ccccc7c6c6c5ccc5ccccc56)c4)cc3)c3ccc4c(c3)oc3ccccc34)c2)cc1] | 764.3 | 64 |

### NMR Data for Some Compounds:

NMR data of compound 193: 1H-NMR (400 MHZ, Methylene-Chloride-D₂) δ ppm 8.00-7.89 (m, 5H), 7.83 (d, 1H), 7.75 (d, 1H), 7.71-7.45 (m, 9H), 7.44-7.25 (m, 7H), 7.21-7.12 (m, 4H), 7.11 (s, 1H), 7.06 (s, 1H), 6.98 (t, 1H), 6.93 (s, 1H), 6.87 (d, 1H), 6.54 (d, 1H);

NMR data of compound 344: 1H-NMR (400 MHZ, Methylene-Chloride-D₂) δ ppm 8.58 (s, 1H), 8.00-7.80 (m, 11H), 7.57 (t, 1H), 7.49-7.32 (m, 9H), 7.27-7.15 (m, 6H), 6.99 (s, 1H), 6.94 (s, 1H), 6.80 (d, 1H), 6.69 (d, 1H), 6.39 (d, 1H); and

NMR data of compound 433: 1H-NMR (400 MHZ, Methylene-Chloride-D₂) δ ppm 7.98 (d, 1H), 7.95-7.87 (m, 3H), 7.85-7.73 (m, 6H), 7.71-7.65 (m, 3H), 7.62-7.44 (m, 6H), 7.43-7.33 (m, 6H), 7.28 (d, 1H), 7.07 (s, 1H), 7.01-6.96 (m, 2H), 6.72 (d, 1H), 6.62 (d, 1H).

### Organic Electroluminescent Device Manufacture and Evaluation:

### Example 1: Manufacture of Red Organic Electroluminescent Device

An anode was pretreated by the following process: surface treatment was performed with UV ozone and O₂:N₂ plasma on an ITO/Ag/ITO substrate with a thickness of 100 Å, 1000 Å, and 100 Å in sequence to increase the work function of the anode, and the surface of the ITO substrate was cleaned with an organic solvent to remove impurities and oil on the surface of the ITO substrate.

PD and α-NPD were co-evaporated on an experimental substrate (the anode) at an evaporation rate ratio of 2%:98% to form a hole injection layer (HIL) having a thickness of 100 Å, and then α-NPD was vacuum-evaporated on the hole injection layer to form a hole transport layer having a thickness of 1060 Å.

A compound HT-2 was vacuum-evaporated on the hole transport layer to form a hole adjustment layer having a thickness of 670 Å.

Next, a compound 6, RH—N and RD-1 were co-evaporated on the hole adjustment layer at an evaporation rate ratio of 49%:49%:2% to form a red electroluminescent layer (EML) with a thickness of 400 Å.

A compound ET-1 and LiQ were mixed in a weight ratio of 1:1 and evaporated on the electroluminescent layer to form an electron transport layer (ETL) having a thickness of 350 Å, Yb was evaporated on the electron transport layer to form an electron injection layer (EIL) having a thickness of 10 Å, and then magnesium (Mg) and silver (Ag) were mixed in an evaporation rate of 1:9 and vacuum-evaporated on the electron injection layer to form a cathode having a thickness of 130 Å.

In addition, CP-1 was vacuum-evaporated on the above cathode to form a capping layer having a thickness of 820 Å, thus completing the manufacture of a red organic electroluminescent device.

### Examples 2 to 66

An organic electroluminescent device was manufactured by the same method as that in Example 1, except that a compound X in Table 7 below was used instead of the compound 6 in Example 1 when the electroluminescent layer was manufactured.

### Comparative Examples 1 to 4

An organic electroluminescent device was manufactured by the same method as that in Example 1, except that a compound A, a compound B, a compound C and a compound D were respectively used instead of the compound 6 in Example 1 when the electroluminescent layer was manufactured.

In the Examples and Comparative examples, the structures of compounds used are as follows:

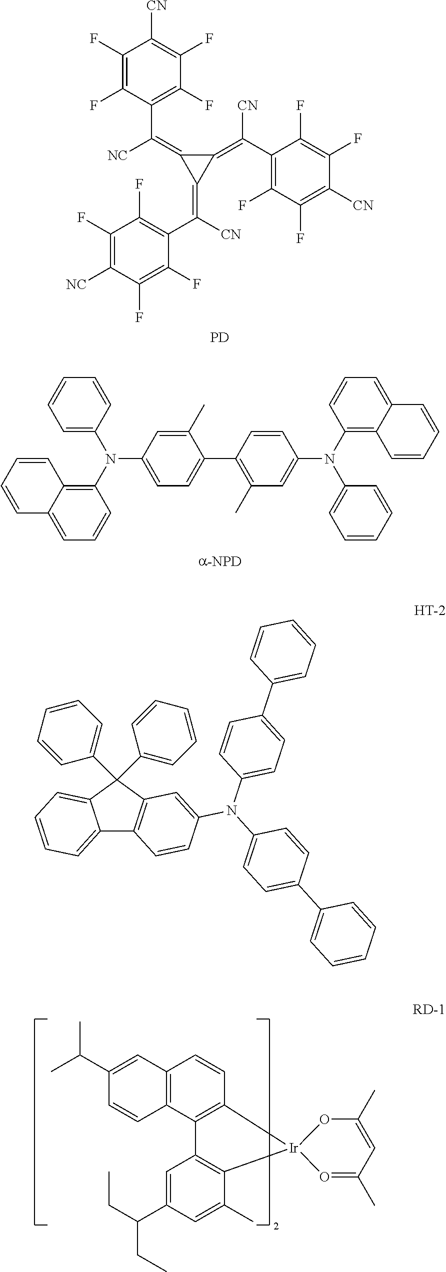

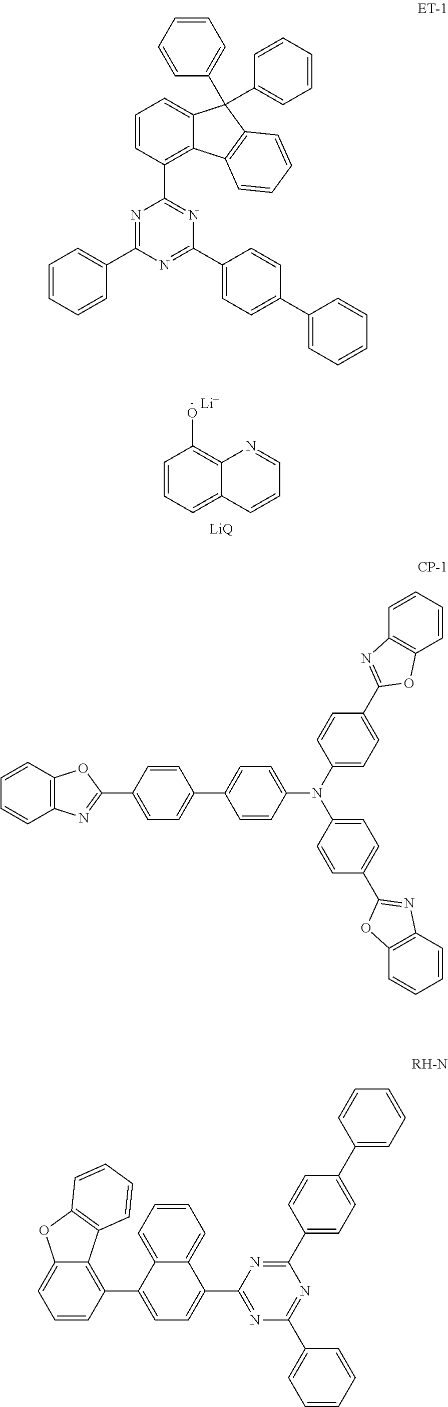

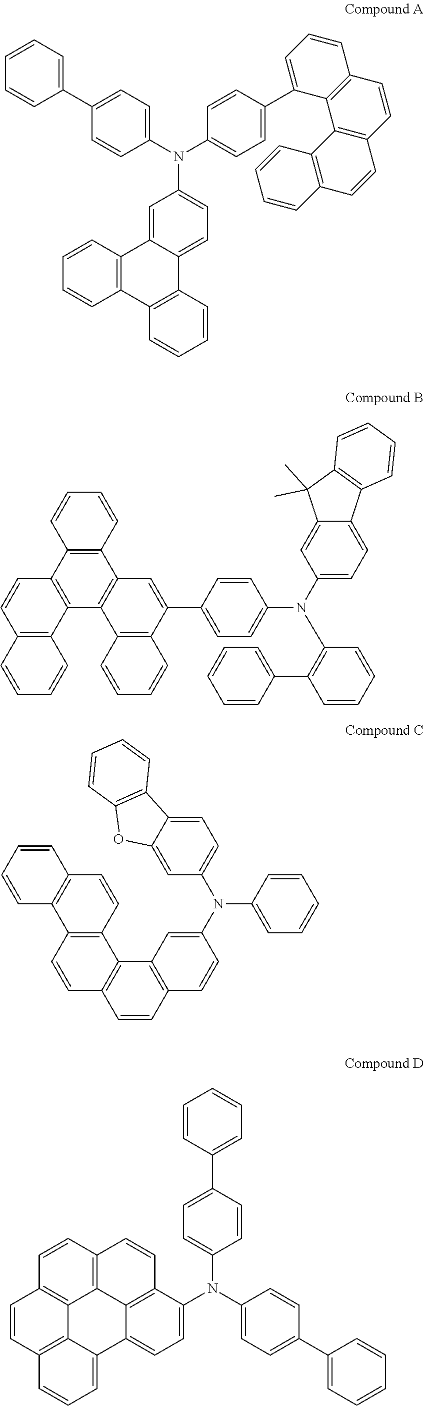

The red organic electroluminescent devices manufactured in Examples 1 to 66 and Comparative examples 1 to 4 were subjected to performance tests, and specifically, the current-voltage-brightness (IVL) performance of the devices was tested under the condition of 10 mA/cm², and the T95 device service life was tested under the condition of 20 mA/cm², and the test results are shown in Table 7.

TABLE 7

| | Electro- | Oper- | | | | |
|---|---|---|---|---|---|---|
| | luminescent | ating | | | | T95 |
| Example | layer compound | voltage | Cd/ | | | (hrs)@20 |
| No. | X:RH-N:RD-1 | (V) | A | CIEx | CIEy | mA/cm² |
| Example 1 | Compound 6 | 3.30 | 53.5 | 0.680 | 0.320 | 448 |
| Example 2 | Compound 9 | 3.46 | 54.4 | 0.680 | 0.320 | 437 |
| Example 3 | Compound 13 | 3.45 | 53.3 | 0.680 | 0.320 | 445 |
| Example 4 | Compound 16 | 3.47 | 53.4 | 0.680 | 0.320 | 449 |
| Example 5 | Compound 17 | 3.50 | 54.2 | 0.680 | 0.320 | 435 |
| Example 6 | Compound 23 | 3.46 | 54.7 | 0.680 | 0.320 | 440 |
| Example 7 | Compound 25 | 3.45 | 53.7 | 0.680 | 0.320 | 448 |
| Example 8 | Compound 54 | 3.29 | 54.4 | 0.680 | 0.320 | 456 |
| Example 9 | Compound 60 | 3.31 | 54.9 | 0.680 | 0.320 | 496 |
| Example 10 | Compound 67 | 3.49 | 53.8 | 0.680 | 0.320 | 447 |
| Example 11 | Compound 71 | 3.48 | 53.4 | 0.680 | 0.320 | 454 |
| Example 12 | Compound 79 | 3.46 | 53.8 | 0.680 | 0.320 | 439 |
| Example 13 | Compound 84 | 3.26 | 53.5 | 0.680 | 0.320 | 458 |
| Example 14 | Compound 87 | 3.50 | 54.0 | 0.680 | 0.320 | 444 |
| Example 15 | Compound 91 | 3.51 | 54.1 | 0.680 | 0.320 | 454 |
| Example 16 | Compound 102 | 3.29 | 53.6 | 0.680 | 0.320 | 438 |
| Example 17 | Compound 109 | 3.47 | 54.5 | 0.680 | 0.320 | 450 |
| Example 18 | Compound 116 | 3.49 | 54.6 | 0.680 | 0.320 | 447 |
| Example 19 | Compound 119 | 3.51 | 53.8 | 0.680 | 0.320 | 439 |
| Example 20 | Compound 122 | 3.49 | 54.5 | 0.680 | 0.320 | 453 |
| Example 21 | Compound 127 | 3.51 | 54.6 | 0.680 | 0.320 | 453 |
| Example 22 | Compound 139 | 3.49 | 54.2 | 0.680 | 0.320 | 483 |
| Example 23 | Compound 143 | 3.45 | 53.1 | 0.680 | 0.320 | 455 |
| Example 24 | Compound 147 | 3.49 | 54.9 | 0.680 | 0.320 | 449 |
| Example 25 | Compound 150 | 3.27 | 53.2 | 0.680 | 0.320 | 449 |
| Example 26 | Compound 162 | 3.29 | 53.9 | 0.680 | 0.320 | 456 |
| Example 27 | Compound 169 | 3.46 | 53.8 | 0.680 | 0.320 | 454 |
| Example 28 | Compound 171 | 3.48 | 54.7 | 0.680 | 0.320 | 441 |
| Example 29 | Compound 177 | 3.48 | 54.1 | 0.680 | 0.320 | 456 |
| Example 30 | Compound 179 | 3.48 | 53.3 | 0.680 | 0.320 | 451 |
| Example 31 | Compound 193 | 3.46 | 53.6 | 0.680 | 0.320 | 457 |
| Example 32 | Compound 433 | 3.46 | 54.2 | 0.680 | 0.320 | 458 |
| Example 33 | Compound 446 | 3.50 | 54.4 | 0.680 | 0.320 | 452 |
| Example 34 | Compound 205 | 3.51 | 61.1 | 0.680 | 0.320 | 413 |
| Example 35 | Compound 216 | 3.28 | 59.7 | 0.680 | 0.320 | 406 |
| Example 36 | Compound 228 | 3.28 | 59.1 | 0.680 | 0.320 | 416 |
| Example 37 | Compound 231 | 3.51 | 61.5 | 0.680 | 0.320 | 411 |
| Example 38 | Compound 241 | 3.50 | 60.3 | 0.680 | 0.320 | 420 |
| Example 39 | Compound 244 | 3.46 | 60.7 | 0.680 | 0.320 | 409 |
| Example 40 | Compound 238 | 3.51 | 60.6 | 0.680 | 0.320 | 404 |
| Example 41 | Compound 250 | 3.46 | 59.2 | 0.680 | 0.320 | 415 |
| Example 42 | Compound 257 | 3.45 | 59.5 | 0.680 | 0.320 | 420 |
| Example 43 | Compound 264 | 3.28 | 61.5 | 0.680 | 0.320 | 423 |
| Example 44 | Compound 265 | 3.50 | 59.7 | 0.680 | 0.320 | 414 |
| Example 45 | Compound 297 | 3.50 | 58.9 | 0.680 | 0.320 | 406 |
| Example 46 | Compound 304 | 3.48 | 62.3 | 0.680 | 0.320 | 411 |
| Example 47 | Compound 307 | 3.50 | 58.7 | 0.680 | 0.320 | 423 |
| Example 48 | Compound 311 | 3.51 | 58.5 | 0.680 | 0.320 | 426 |
| Example 49 | Compound 314 | 3.31 | 61.0 | 0.680 | 0.320 | 420 |
| Example 50 | Compound 327 | 3.48 | 61.1 | 0.680 | 0.320 | 412 |
| Example 51 | Compound 334 | 3.45 | 61.1 | 0.680 | 0.320 | 413 |
| Example 52 | Compound 339 | 3.50 | 61.4 | 0.680 | 0.320 | 402 |
| Example 53 | Compound 344 | 3.31 | 60.6 | 0.680 | 0.320 | 403 |
| Example 54 | Compound 355 | 3.46 | 60.6 | 0.680 | 0.320 | 401 |
| Example 55 | Compound 368 | 3.28 | 59.8 | 0.680 | 0.320 | 412 |
| Example 56 | Compound 372 | 3.49 | 61.6 | 0.680 | 0.320 | 421 |
| Example 57 | Compound 375 | 3.48 | 62.2 | 0.680 | 0.320 | 417 |
| Example 58 | Compound 376 | 3.45 | 60.1 | 0.680 | 0.320 | 419 |
| Example 59 | Compound 382 | 3.48 | 59.0 | 0.680 | 0.320 | 428 |
| Example 60 | Compound 383 | 3.46 | 62.1 | 0.680 | 0.320 | 402 |
| Example 61 | Compound 405 | 3.51 | 59.5 | 0.680 | 0.320 | 407 |
| Example 62 | Compound 416 | 3.45 | 60.4 | 0.680 | 0.320 | 423 |
| Example 63 | Compound 419 | 3.50 | 59.0 | 0.680 | 0.320 | 422 |
| Example 64 | Compound 462 | 3.44 | 59.9 | 0.680 | 0.320 | 405 |
| Example 65 | Compound 466 | 3.43 | 59.3 | 0.680 | 0.320 | 410 |
| Example 66 | Compound 489 | 3.50 | 59.5 | 0.680 | 0.320 | 418 |
| Comparative | Compound A | 3.52 | 48.2 | 0.680 | 0.320 | 350 |
| example 1 | | | | | | |
| Comparative | Compound B | 3.51 | 47.3 | 0.680 | 0.320 | 354 |
| example 2 | | | | | | |
| Comparative | Compound C | 3.48 | 46.9 | 0.680 | 0.320 | 330 |
| example 3 | | | | | | |
| Comparative | Compound D | 3.53 | 45.1 | 0.680 | 0.320 | 327 |
| example 4 | | | | | | |

As can be seen from the above Table 7, when the compound of the disclosure is used as a host material of the red organic electroluminescent device in Examples 1 to 66, the luminous efficiency s improved by at least 10.4%, and the service life is improved by at least 13.3% compared with Comparative examples 1 to 4.

The structure of the compound of the disclosure includes a compound in which [5]helicene is used as a parent nucleus to be linked with arylamine, [5]helicene has a large conjugation plane and rigidity, which is conducive to intermolecular stacking, and linking [5]helicene to arylamine helps to improve the hole mobility of materials; meanwhile, in addition, two benzene rings at the end of [5]helicene are not in the same plane due to the steric hindrance effect of hydrogen atoms, and stacking between molecules can be inhibited to a certain extent, improving the film-forming properties of the materials; and a combination of [5]helicene as the parent nucleus and a triarylamine group can also make a first triplet energy level (T1) of the compound as a whole at a suitable level, which facilitates energy transfer in the electroluminescent layer, and avoids aggregation between molecules. When the compound of the disclosure is used as a hole transport type host material in a hybrid type host material, the balance of carriers in a electroluminescent layer can be improved, the carrier utilization rate can be improved, and a recombination region of carriers can be broadened, thus significantly improving the efficiency and service life of devices.

The preferred embodiments of the disclosure are described in detail in combination with the drawings, but the disclosure is not limited to the specific details in the above embodiments. Various simple variations can be made to the technical solutions of the disclosure in the scope of the technical concept of the disclosure, and these simple variations all fall within the protection scope of the disclosure.

1. An arylamine compound, having a structure represented by a formula 1: 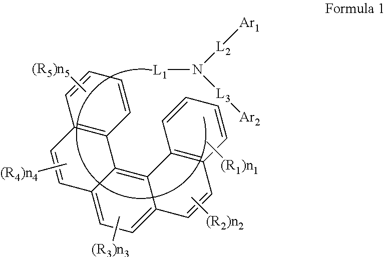
wherein L₁, L₂ and L₃ are the same or different, and are each independently selected from a single bond, substituted or unsubstituted arylene with 6 to 30 carbon atoms, and substituted or unsubstituted heteroarylene with 3 to 30 carbon atoms;
Ar₁ and Ar₂ are the same or different, and are each independently selected from substituted or unsubstituted aryl with 6 to 30 carbon atoms, and substituted or unsubstituted heteroaryl with 3 to 30 carbon atoms;
R₁, R₂, R₃, R₄ and R₅ are the same or different, and are each independently selected from hydrogen, deuterium, cyano, aryl with 6 to 20 carbon atoms, heteroaryl with 3 to 20 carbon atoms, alkyl with 1 to 10 carbon atoms, alkoxy with 1 to 10 carbon atoms, haloalkyl with 1 to 10 carbon atoms, trialkylsilyl with 3 to 12 carbon atoms, triphenylsilyl, cycloalkyl with 3 to 10 carbon atoms, and deuteroalkyl with 1 to 10 carbon atoms;
n₁ and n₅ are each independently selected from 0, 1, 2, 3 or 4; and n₂, n₃ and n₄ are each independently selected from 0, 1 or 2; and
substituent(s) in L₁, L₂, L₃, Ar₁ and Ar₂ are the same or different, and are each independently selected from deuterium, fluorine, cyano, aryl with 6 to 20 carbon atoms, heteroaryl with 3 to 20 carbon atoms, alkyl with 1 to 10 carbon atoms, alkoxy with 1 to 10 carbon atoms, haloalkyl with 1 to 10 carbon atoms, trialkylsilyl with 3 to 12 carbon atoms, triphenylsilyl, cycloalkyl with 3 to 10 carbon atoms, and deuteroalkyl with 1 to 10 carbon atoms; and optionally, any two adjacent substituents in Ar₁ and Ar₂ form a saturated or unsaturated 3- to 15-membered ring.

2. The arylamine compound according to claim 1, wherein Ar₁ and Ar₂ are each independently selected from substituted or unsubstituted aryl with 6 to 25 carbon atoms, and substituted or unsubstituted heteroaryl with 12 to 18 carbon atoms.

3. The arylamine compound according to claim 1, wherein Ar₁ and Ar₂ are each independently selected from a substituted or unsubstituted group W, the unsubstituted group W being selected from the group consisting of the following groups: 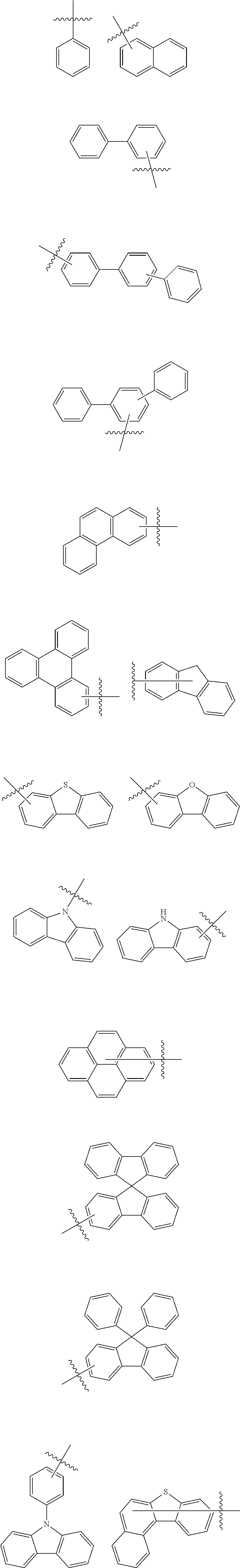 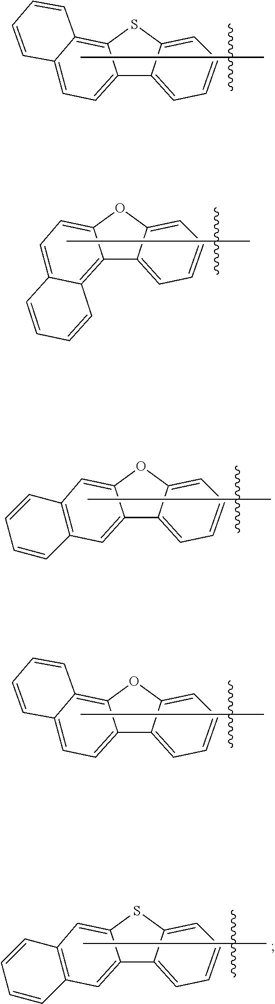
and
the substituted group W has one or two or more substituents each independently selected from deuterium, fluorine, cyano, trimethylsilyl, trifluoromethyl, cyclopentyl, cyclohexyl, methyl, ethyl, isopropyl, tert-butyl, phenyl, naphthyl, pyridyl, dibenzofuranyl, dibenzothienyl, and carbazolyl, and when the number of substituents in the group W is greater than 1, the substituents are the same or different.

4. The arylamine compound according to claim 1, wherein Ar₁ and Ar₂ are each independently selected from the following groups:

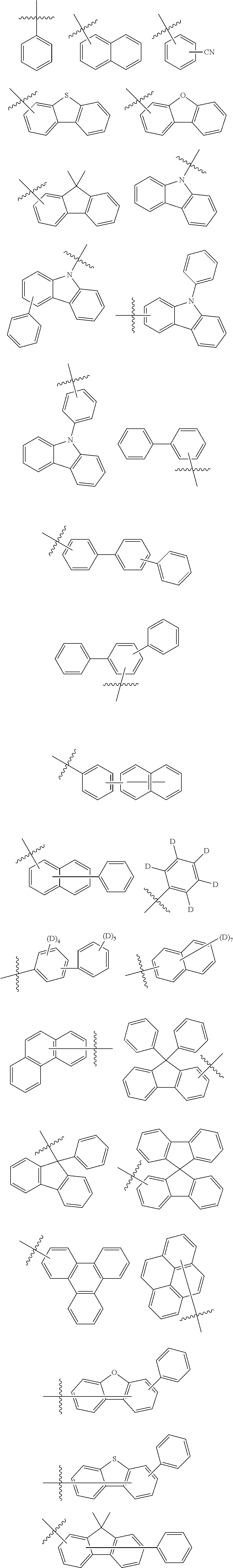

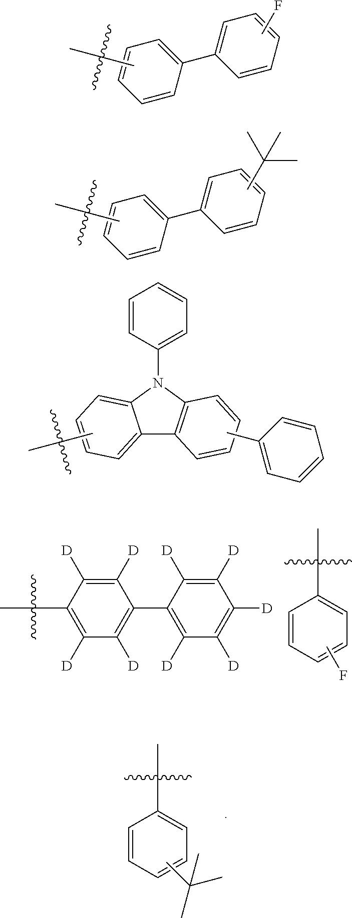

5. The arylamine compound according to claim 1, wherein L₁, L₂ and L₃ are each independently selected from a single bond, substituted or unsubstituted arylene with 6 to 15 carbon atoms, and substituted or unsubstituted heteroarylene with 12 to 18 carbon atoms.

6. The arylamine compound according to claim 1, wherein L₁, L₂ and L₃ are each independently selected from a single bond, substituted or unsubstituted phenylene, substituted or unsubstituted naphthylene, substituted or unsubstituted biphenylene, substituted or unsubstituted fluorenylene, substituted or unsubstituted phenanthrylene, substituted or unsubstituted anthrylene, substituted or unsubstituted carbazolylene, substituted or unsubstituted dibenzothienylene, and substituted or unsubstituted dibenzofurylene.

7. The arylamine compound according to claim 1, wherein L₁ is selected from a single bond or the group consisting of the following groups: 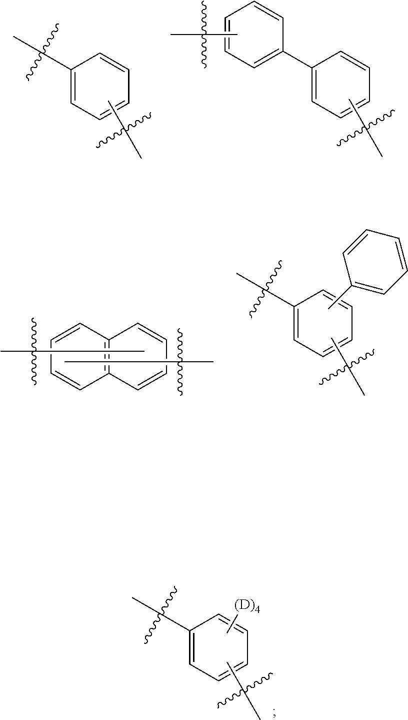
and
L₂ and L₃ are each independently selected from a single bond or the group consisting of the following groups:

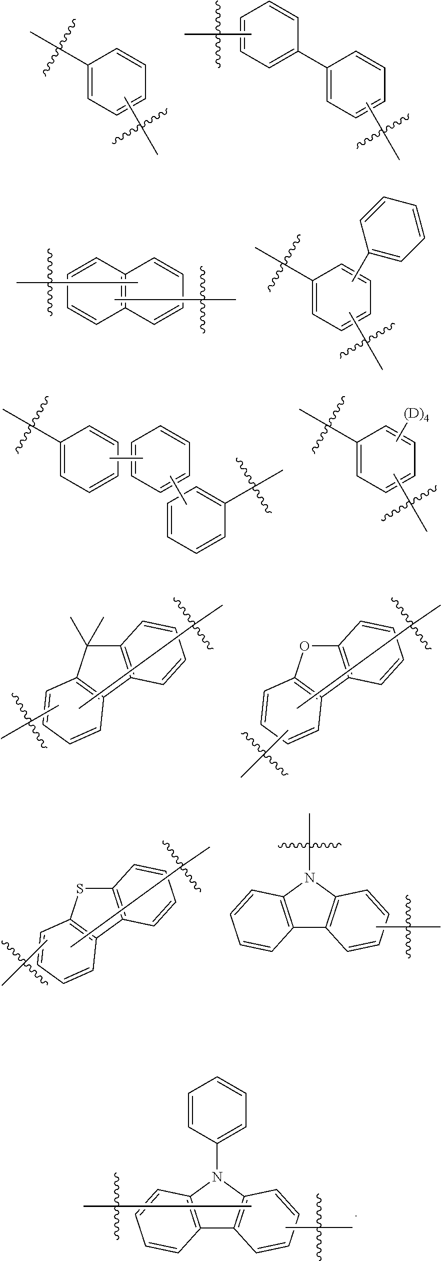

8. The arylamine compound according to claim 1, wherein 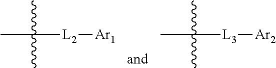
are each independently selected from the following groups:

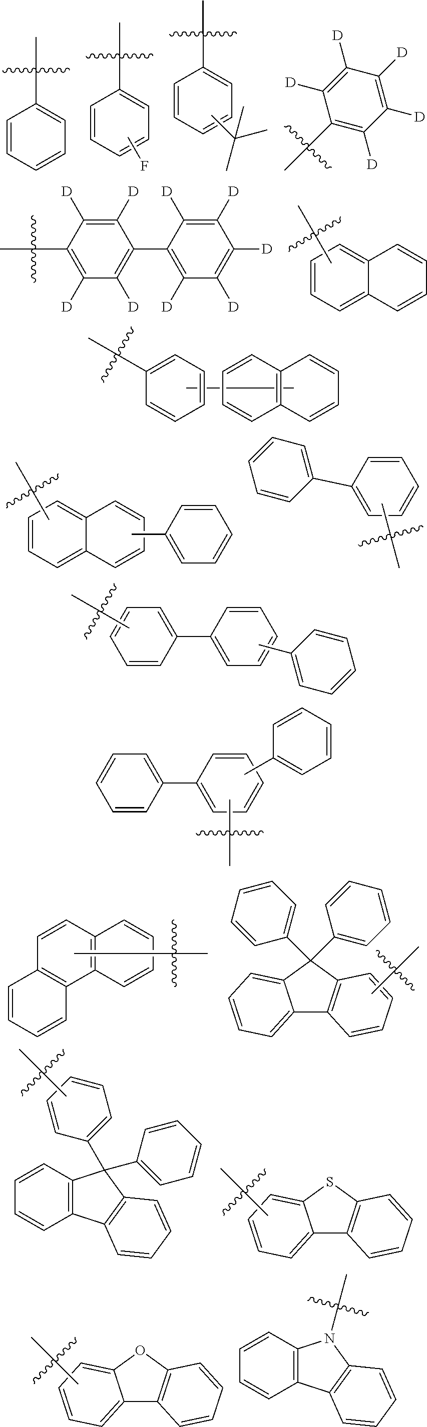

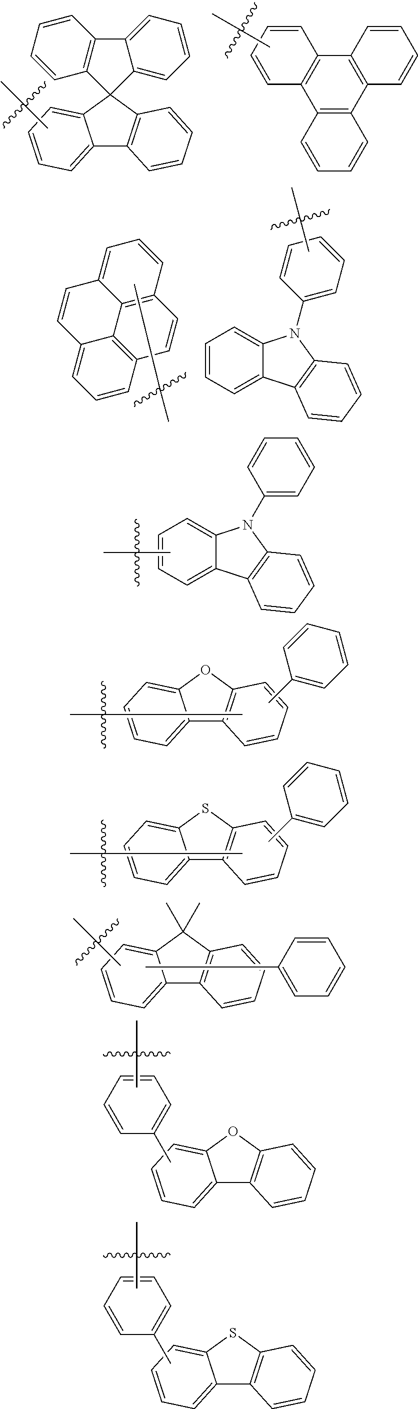

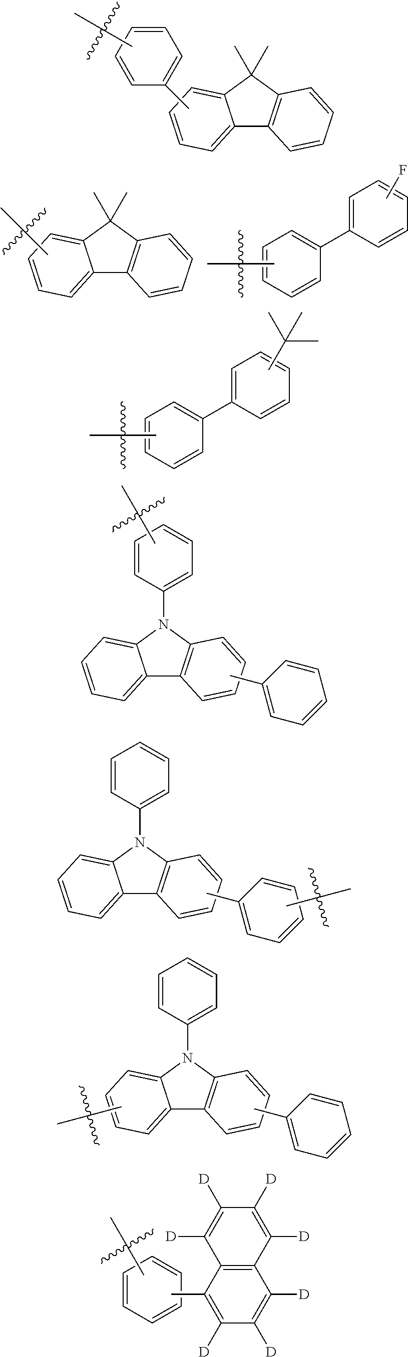

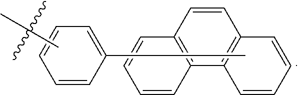

9. The arylamine compound according to claim 1, wherein L₁ is selected from a single bond or the following groups: 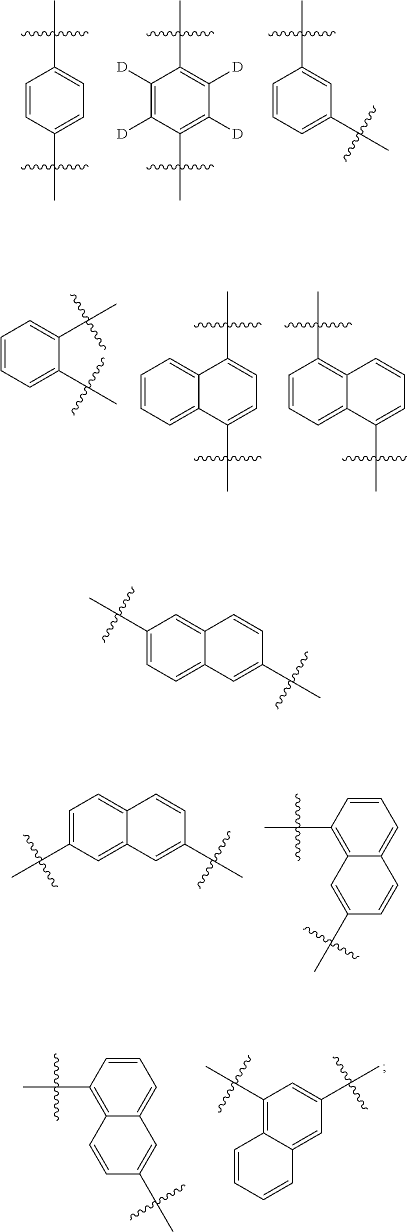
and
L₂ and Ls are each independently selected from a single bond or the following groups:

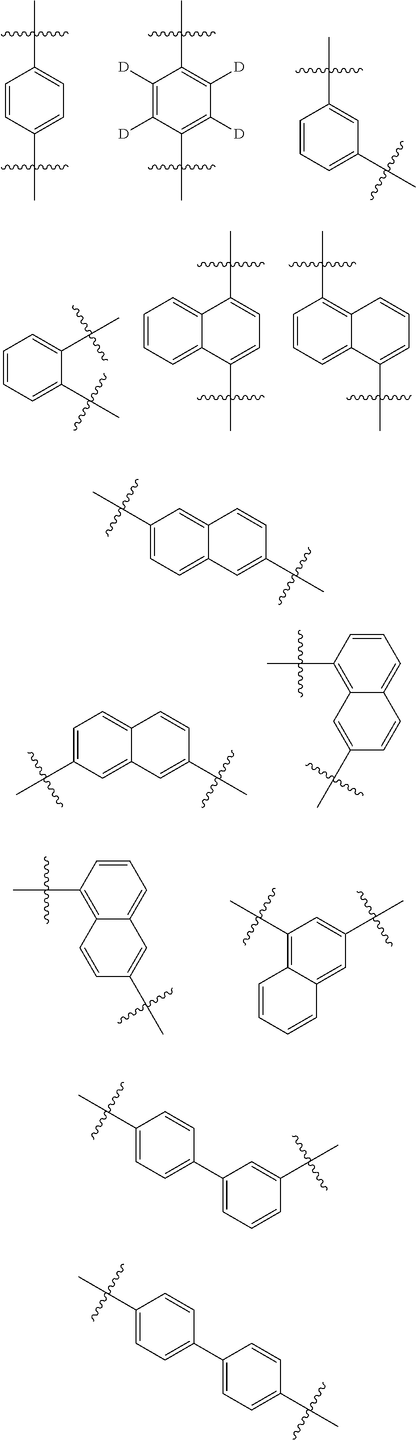

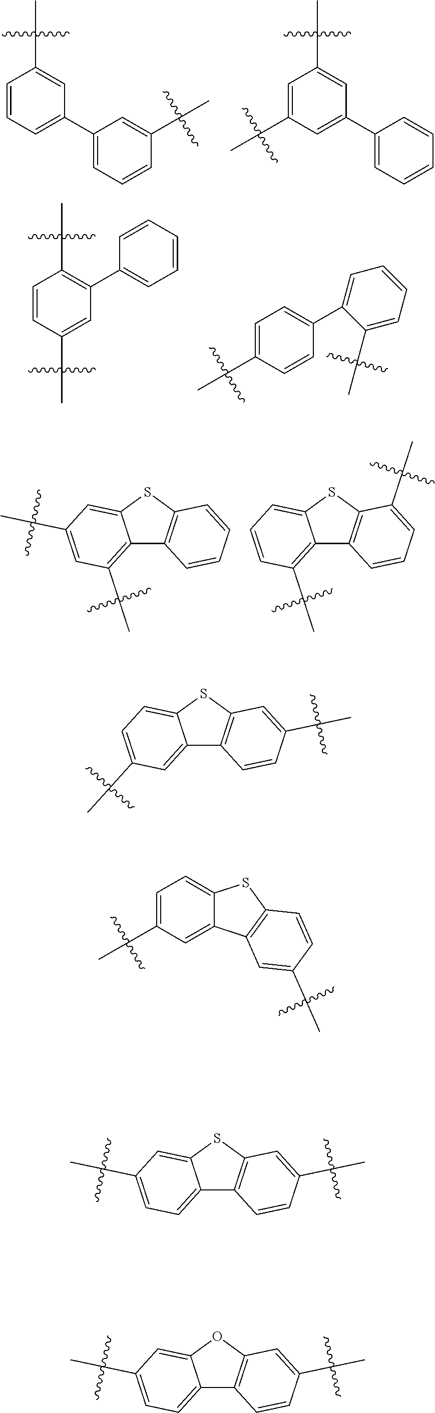

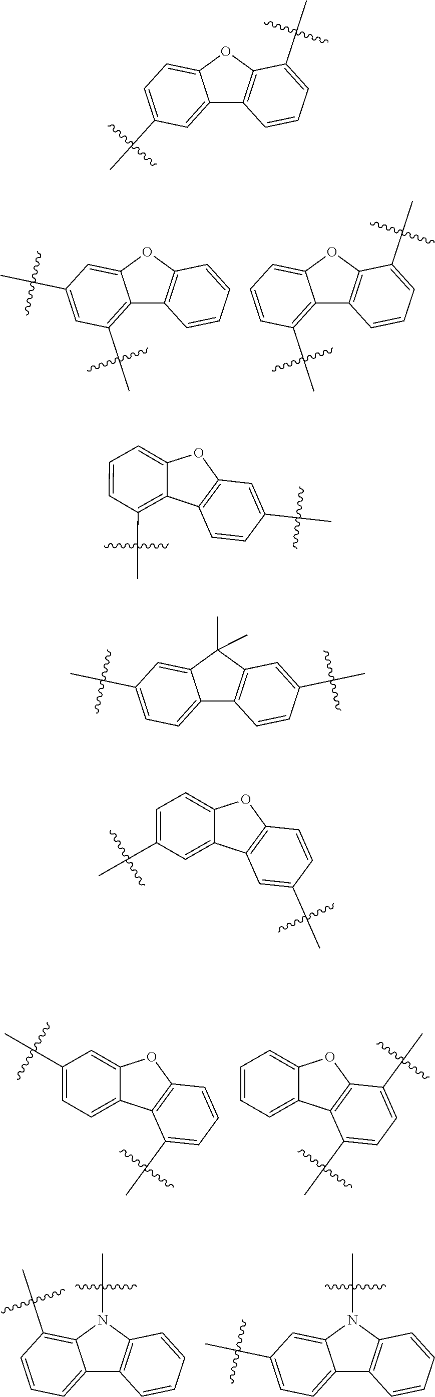

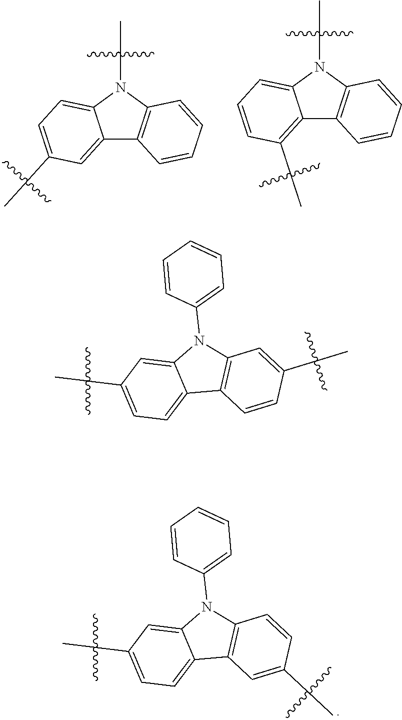

10. The arylamine compound according to claim 1, wherein 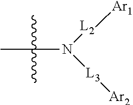
is selected from the following groups:

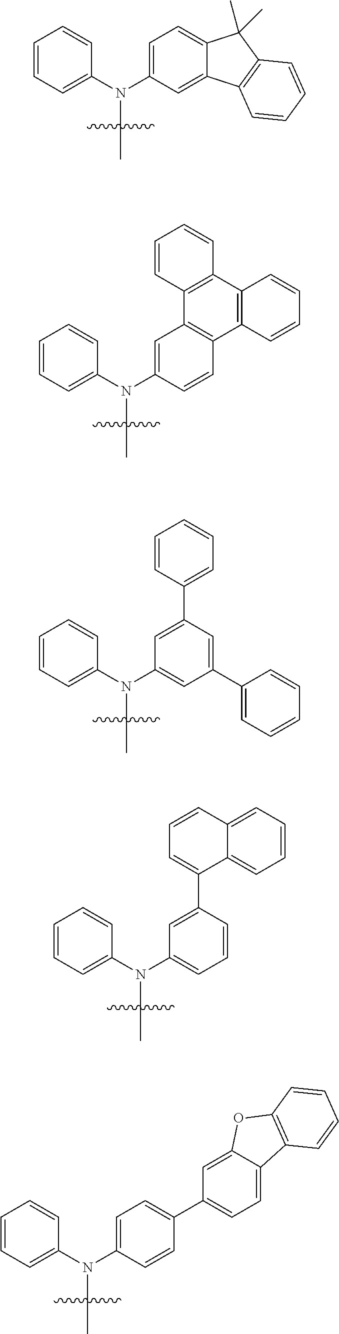 [SMILES: *N(c1ccccc1)c1ccc2c(c1)-c1ccccc1C2(C)C ; *N(c1ccccc1)c1ccc2c3ccccc3c3ccccc3c2c1 ; *N(c1ccccc1)c1cc(-c2ccccc2)cc(-c2ccccc2)c1 ; *N(c1ccccc1)c1cccc(-c2cccc3ccccc23)c1 ; *N(c1ccccc1)c1ccc(-c2ccc3c(c2)oc2ccccc23)cc1]

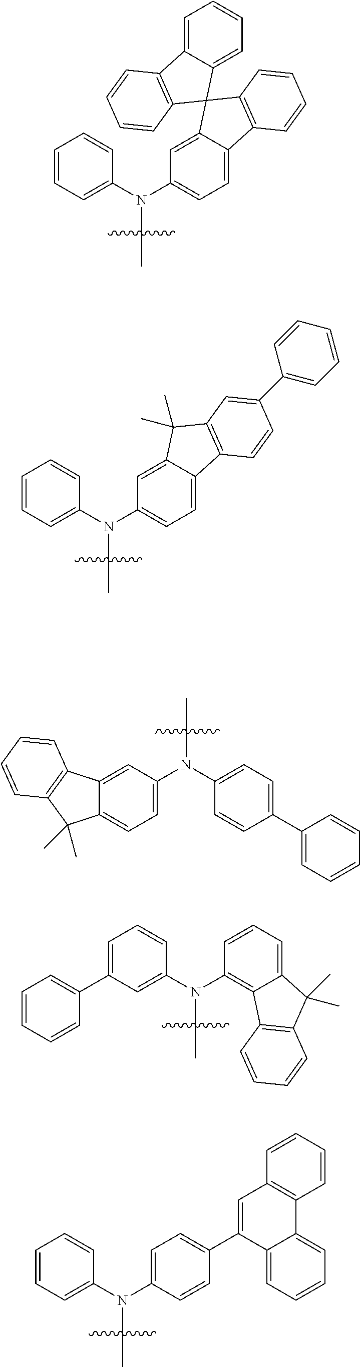 [SMILES: *N(c1ccccc1)c1ccc2c(c1)C1(c3ccccc3-c3ccccc31)c1ccccc1-2 ; *N(c1ccccc1)c1ccc2c(c1)C(C)(C)c1cc(-c3ccccc3)ccc1-2 ; *N(c1ccc(-c2ccccc2)cc1)c1ccc2c(c1)-c1ccccc1C2(C)C ; *N(c1cccc(-c2ccccc2)c1)c1cccc2c1-c1ccccc1C2(C)C ; *N(c1ccccc1)c1ccc(-c2cc3ccccc3c3ccccc23)cc1]

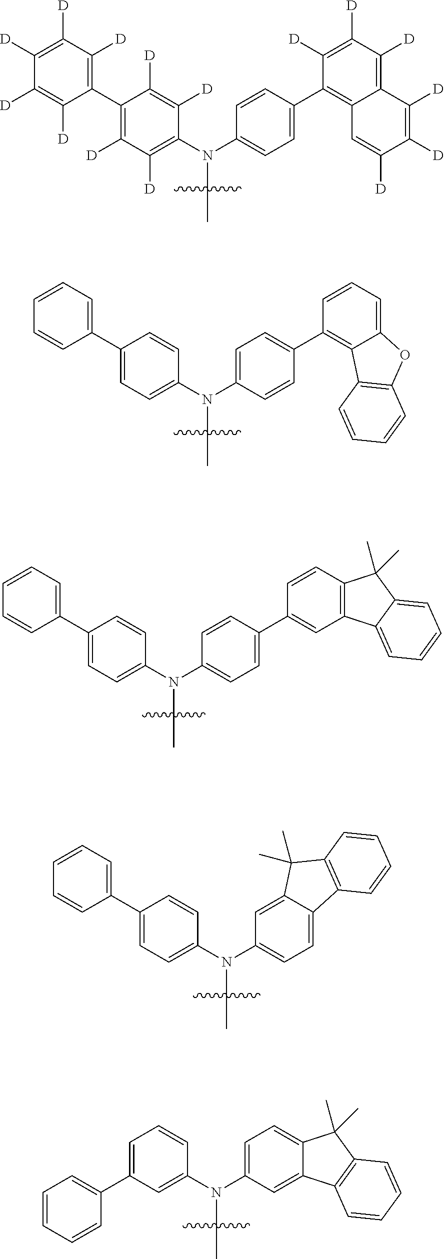 [SMILES: *N(c1ccc(-c2c([2H])c([2H])c([2H])c3c([2H])c([2H])c([2H])cc23)cc1)c1c([2H])c([2H])c(-c2c([2H])c([2H])c([2H])c([2H])c2[2H])c([2H])c1[2H] ; *N(c1ccc(-c2ccccc2)cc1)c1ccc(-c2cccc3oc4ccccc4c23)cc1 ; *N(c1ccc(-c2ccccc2)cc1)c1ccc(-c2ccc3c(c2)-c2ccccc2C3(C)C)cc1 ; *N(c1ccc(-c2ccccc2)cc1)c1ccc2c(c1)C(C)(C)c1ccccc1-2 ; *N(c1cccc(-c2ccccc2)c1)c1ccc2c(c1)-c1ccccc1C2(C)C]

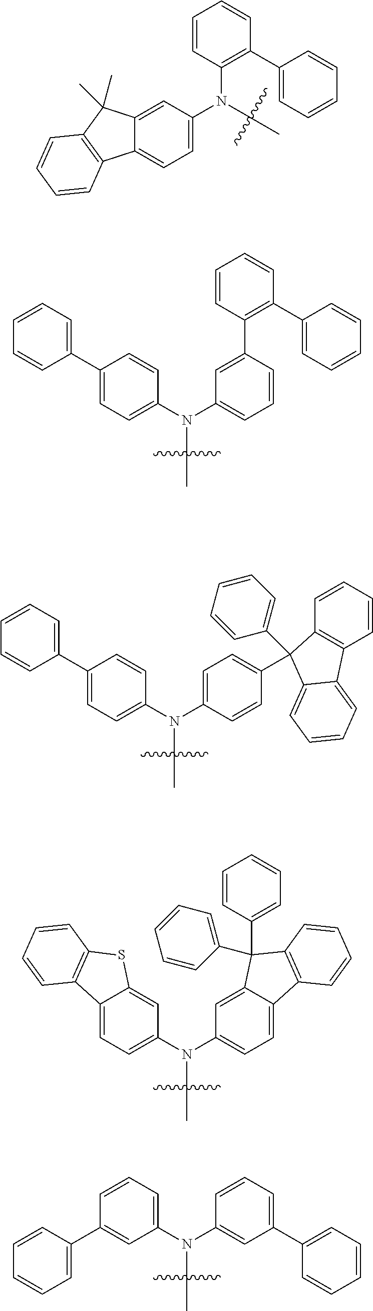 [SMILES: *N(c1ccc2c(c1)C(C)(C)c1ccccc1-2)c1ccccc1-c1ccccc1 ; *N(c1ccc(-c2ccccc2)cc1)c1cccc(-c2ccccc2-c2ccccc2)c1 ; *N(c1ccc(-c2ccccc2)cc1)c1ccc(C2(c3ccccc3)c3ccccc3-c3ccccc32)cc1 ; *N(c1ccc2c(c1)C(c1ccccc1)(c1ccccc1)c1ccccc1-2)c1ccc2c(c1)sc1ccccc12 ; *N(c1cccc(-c2ccccc2)c1)c1cccc(-c2ccccc2)c1]

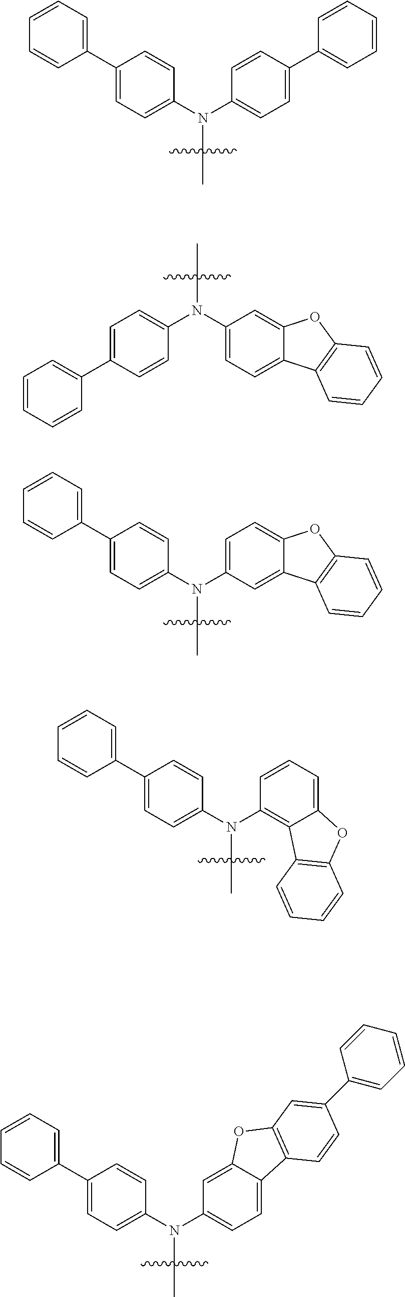

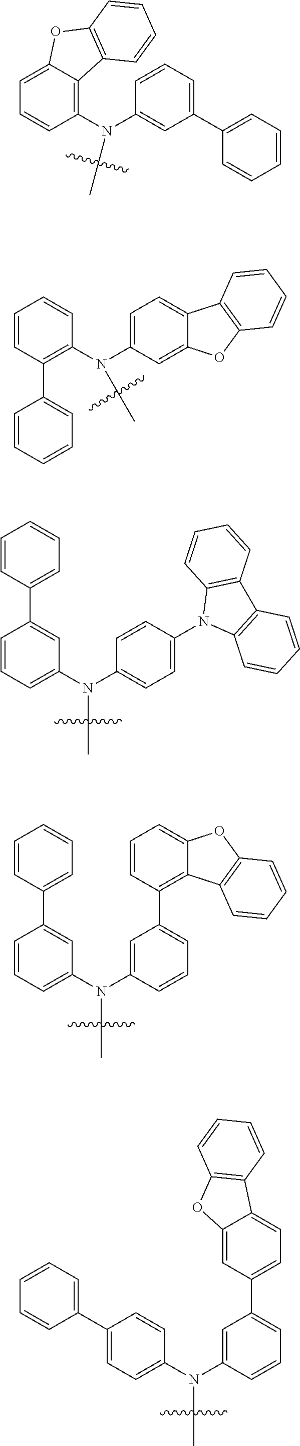 [SMILES: *N(c1cccc(-c2ccccc2)c1)c1cccc2oc3ccccc3c12 ; *N(c1ccc2c(c1)oc1ccccc12)c1ccccc1-c1ccccc1 ; *N(c1ccc(-n2c3ccccc3c3ccccc32)cc1)c1cccc(-c2ccccc2)c1 ; *N(c1cccc(-c2ccccc2)c1)c1cccc(-c2cccc3oc4ccccc4c23)c1 ; *N(c1ccc(-c2ccccc2)cc1)c1cccc(-c2ccc3c(c2)oc2ccccc23)c1]

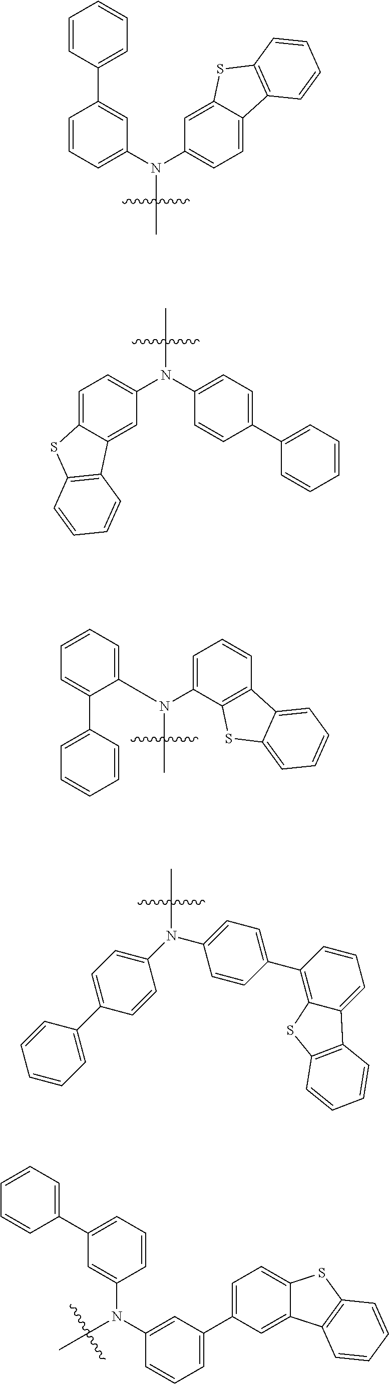 [SMILES: *N(c1cccc(-c2ccccc2)c1)c1ccc2c(c1)sc1ccccc12 ; *N(c1ccc(-c2ccccc2)cc1)c1ccc2sc3ccccc3c2c1 ; *N(c1ccccc1-c1ccccc1)c1cccc2c1sc1ccccc12 ; *N(c1ccc(-c2ccccc2)cc1)c1ccc(-c2cccc3c2sc2ccccc23)cc1 ; *N(c1cccc(-c2ccccc2)c1)c1cccc(-c2ccc3sc4ccccc4c3c2)c1]

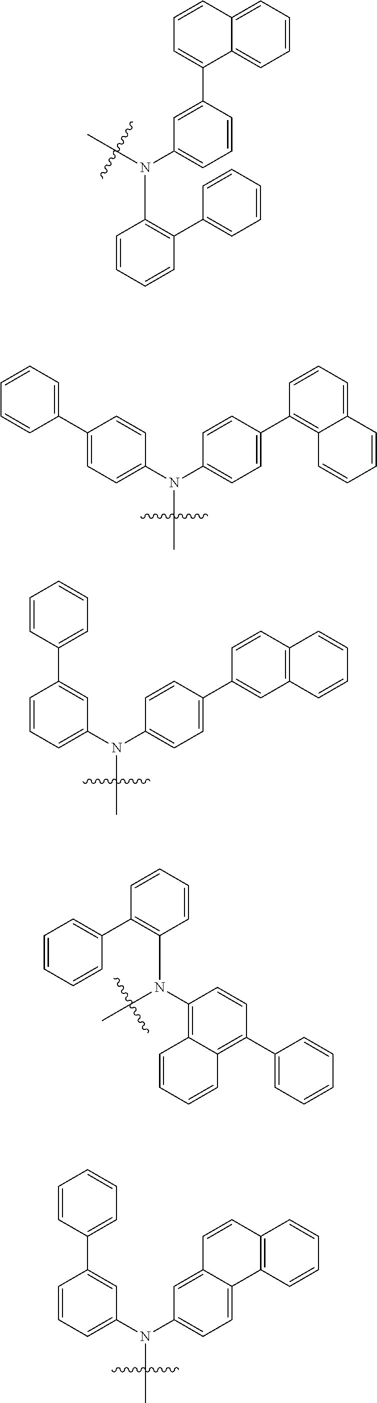 [SMILES: *N(c1cccc(-c2cccc3ccccc23)c1)c1ccccc1-c1ccccc1 ; *N(c1ccc(-c2ccccc2)cc1)c1ccc(-c2cccc3ccccc23)cc1 ; *N(c1ccc(-c2ccc3ccccc3c2)cc1)c1cccc(-c2ccccc2)c1 ; *N(c1ccccc1-c1ccccc1)c1ccc(-c2ccccc2)c2ccccc12 ; *N(c1cccc(-c2ccccc2)c1)c1ccc2c(ccc3ccccc32)c1]

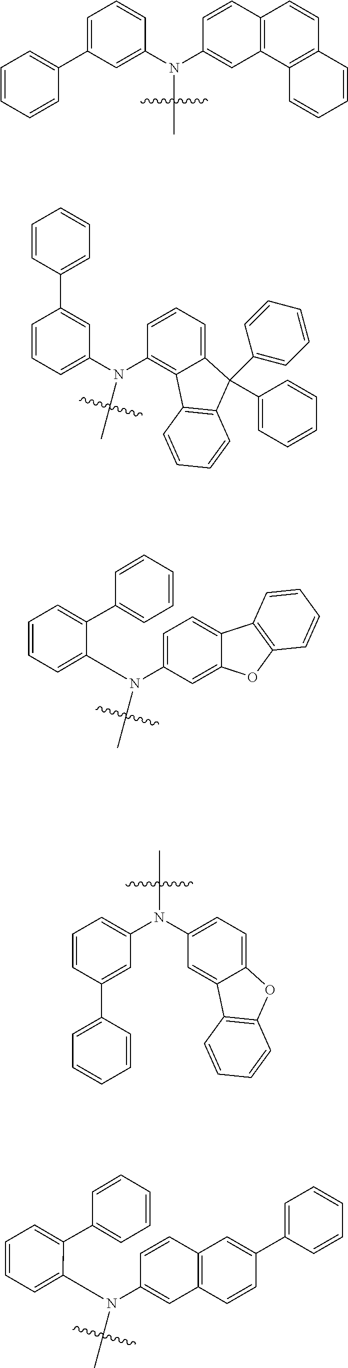 [SMILES: *N(c1cccc(-c2ccccc2)c1)c1ccc2ccc3ccccc3c2c1 ; *N(c1cccc(-c2ccccc2)c1)c1cccc2c1-c1ccccc1C2(c1ccccc1)c1ccccc1 ; *N(c1ccc2c(c1)oc1ccccc12)c1ccccc1-c1ccccc1 ; *N(c1cccc(-c2ccccc2)c1)c1ccc2oc3ccccc3c2c1 ; *N(c1ccc2cc(-c3ccccc3)ccc2c1)c1ccccc1-c1ccccc1]

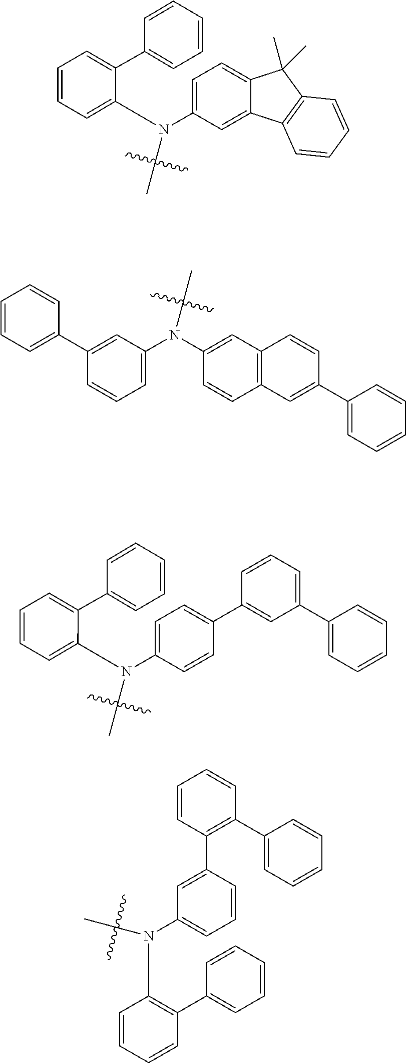 [SMILES: *N(c1ccc2c(c1)-c1ccccc1C2(C)C)c1ccccc1-c1ccccc1 ; *N(c1cccc(-c2ccccc2)c1)c1ccc2cc(-c3ccccc3)ccc2c1 ; *N(c1ccc(-c2cccc(-c3ccccc3)c2)cc1)c1ccccc1-c1ccccc1 ; *N(c1cccc(-c2ccccc2-c2ccccc2)c1)c1ccccc1-c1ccccc1]

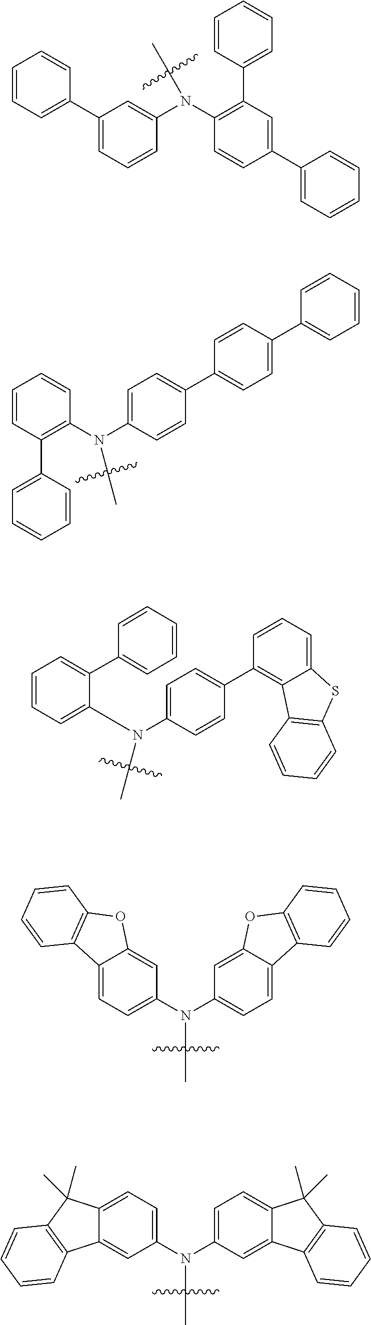 [SMILES: *N(c1cccc(-c2ccccc2)c1)c1ccc(-c2ccccc2)cc1-c1ccccc1 ; *N(c1ccc(-c2ccc(-c3ccccc3)cc2)cc1)c1ccccc1-c1ccccc1 ; *N(c1ccc(-c2cccc3sc4ccccc4c23)cc1)c1ccccc1-c1ccccc1 ; *N(c1ccc2c(c1)oc1ccccc12)c1ccc2c(c1)oc1ccccc12 ; *N(c1ccc2c(c1)-c1ccccc1C2(C)C)c1ccc2c(c1)-c1ccccc1C2(C)C]

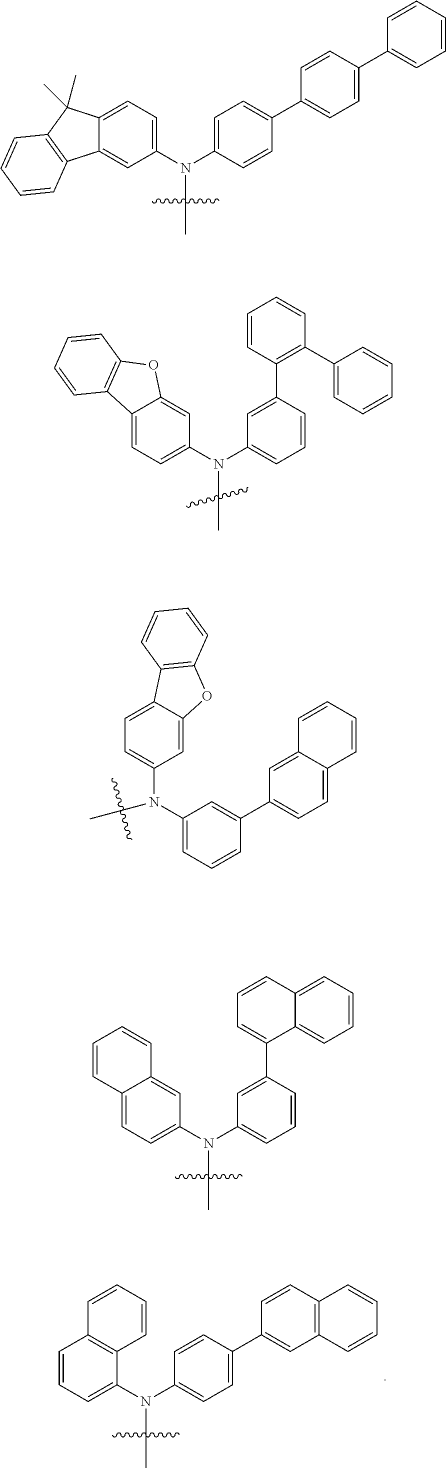 [SMILES: *N(c1ccc(-c2ccc(-c3ccccc3)cc2)cc1)c1ccc2c(c1)-c1ccccc1C2(C)C ; *N(c1cccc(-c2ccccc2-c2ccccc2)c1)c1ccc2c(c1)oc1ccccc12 ; *N(c1cccc(-c2ccc3ccccc3c2)c1)c1ccc2c(c1)oc1ccccc12 ; *N(c1cccc(-c2cccc3ccccc23)c1)c1ccc2ccccc2c1 ; *N(c1ccc(-c2ccc3ccccc3c2)cc1)c1cccc2ccccc12]

11. The arylamine compound according to claim 1, wherein each R₁, each R₂, each R₃, each R₄ and each R₅ are the same or different, and are each independently selected from deuterium, cyano, trideuteromethyl, trimethylsilyl, trifluoromethyl, methyl, ethyl, isopropyl, tert-butyl, phenyl or naphthyl.

12. The arylamine compound according to claim 1, wherein the arylamine compound is selected from the group consisting of the following compounds:

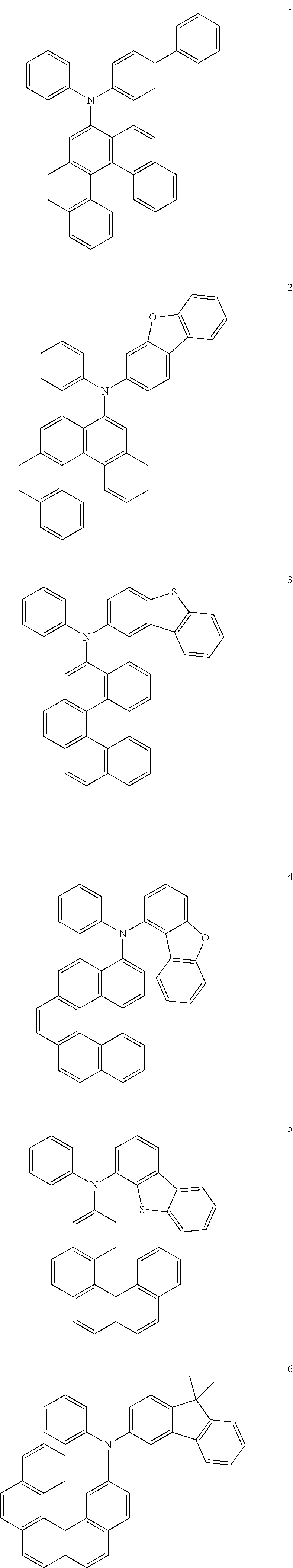 [SMILES: c1ccc(-c2ccc(N(c3ccccc3)c3cc4ccc5ccccc5c4c4c3ccc3ccccc34)cc2)cc1 ; c1ccc(N(c2ccc3c(c2)oc2ccccc23)c2cc3ccccc3c3c2ccc2ccc4ccccc4c23)cc1 ; c1ccc(N(c2ccc3sc4ccccc4c3c2)c2cc3ccc4ccc5ccccc5c4c3c3ccccc23)cc1 ; c1ccc(N(c2cccc3c2ccc2ccc4ccc5ccccc5c4c23)c2cccc3oc4ccccc4c23)cc1 ; c1ccc(N(c2ccc3c(ccc4ccc5ccc6ccccc6c5c43)c2)c2cccc3c2sc2ccccc23)cc1 ; CC1(C)c2ccccc2-c2cc(N(c3ccccc3)c3ccc4ccc5ccc6ccc7ccccc7c6c5c4c3)ccc21]

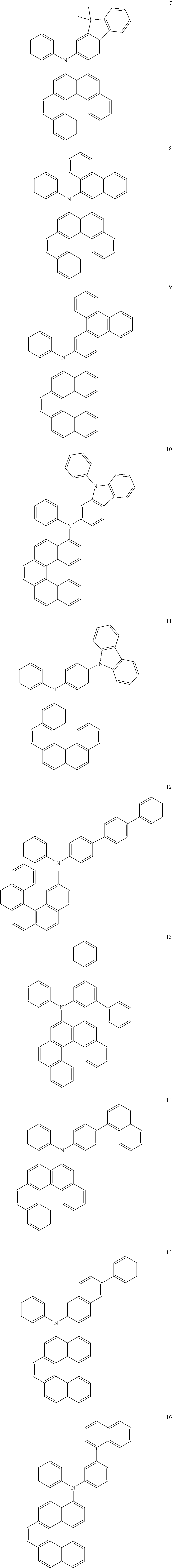

[chemical structure]

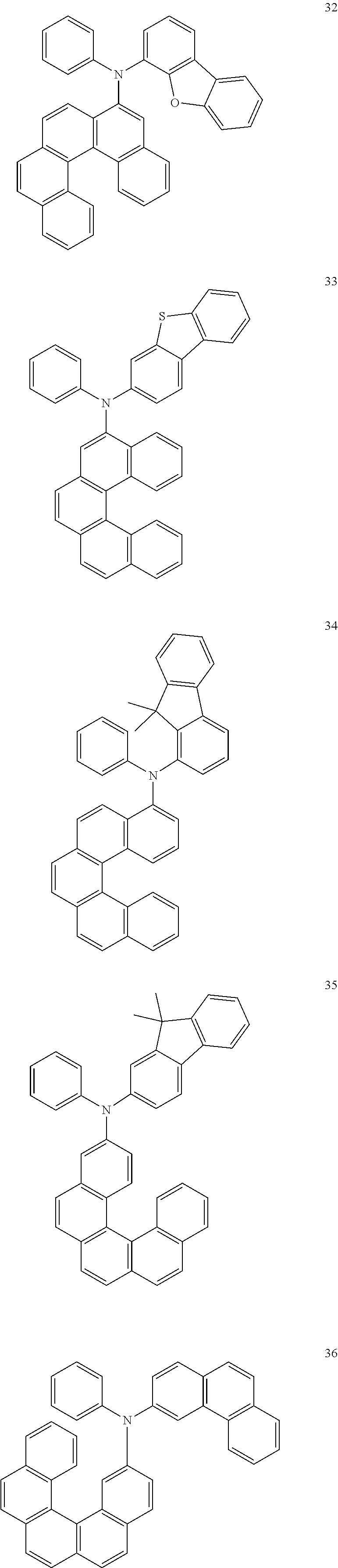 [SMILES: c1ccc(N(c2cc3ccccc3c3c2ccc2ccc4ccccc4c23)c2cccc3c2oc2ccccc23)cc1 ; c1ccc(N(c2ccc3c(c2)sc2ccccc23)c2cc3ccc4ccc5ccccc5c4c3c3ccccc23)cc1 ; CC1(C)c2ccccc2-c2cccc(N(c3ccccc3)c3cccc4c3ccc3ccc5ccc6ccccc6c5c34)c21 ; CC1(C)c2ccccc2-c2ccc(N(c3ccccc3)c3ccc4c(ccc5ccc6ccc7ccccc7c6c54)c3)cc21 ; c1ccc(N(c2ccc3ccc4ccccc4c3c2)c2ccc3ccc4ccc5ccc6ccccc6c5c4c3c2)cc1]

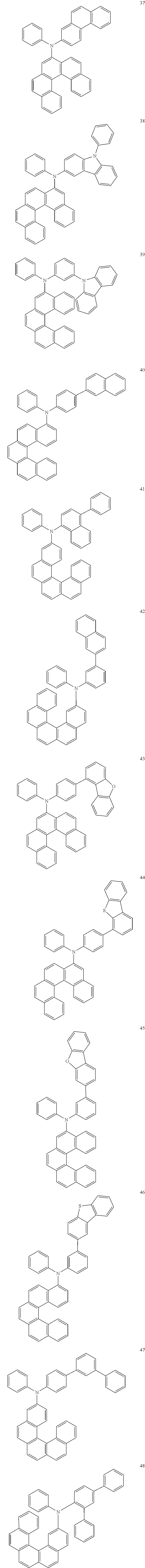

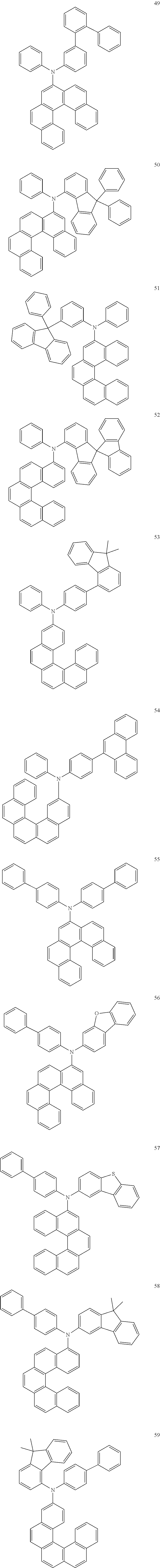

[chemical structure]

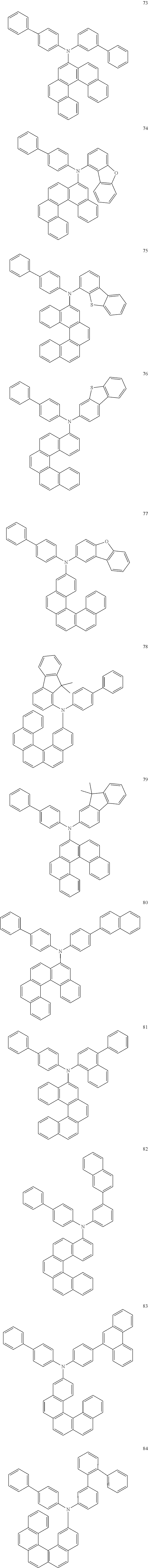

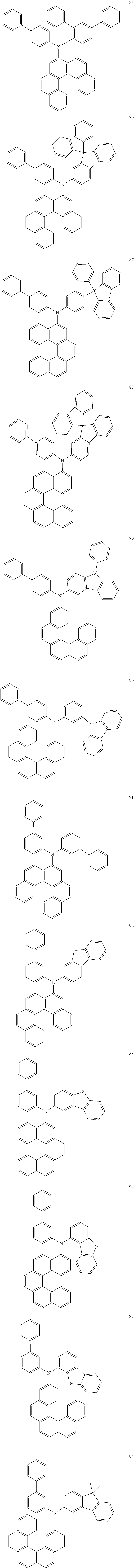

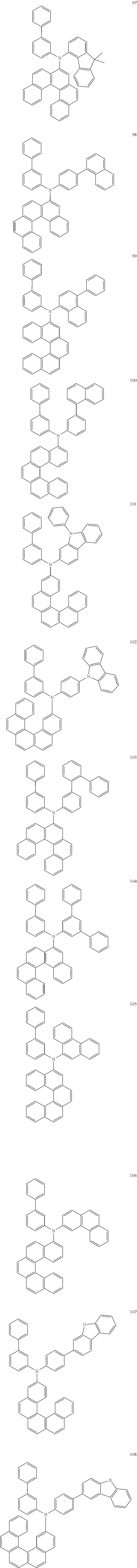

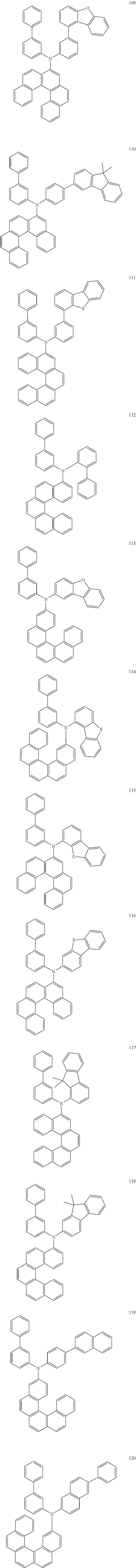

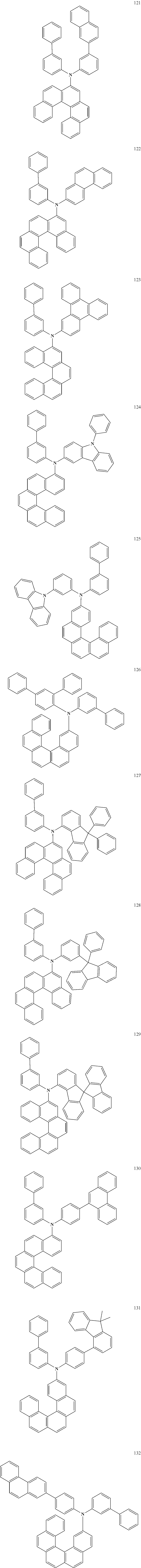

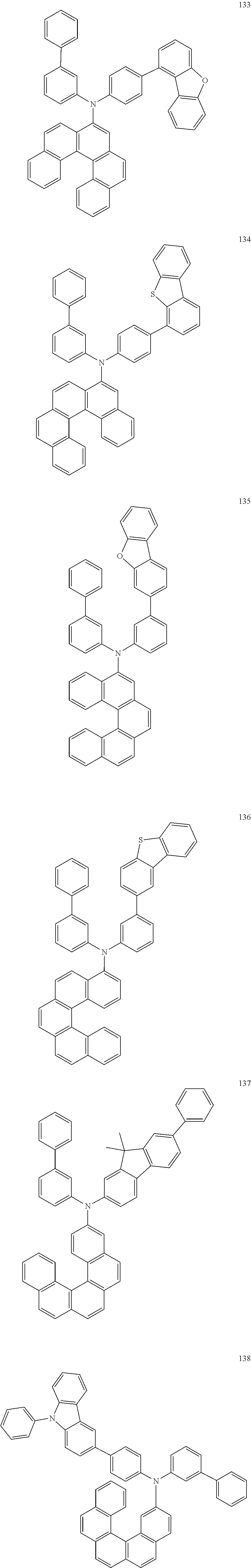

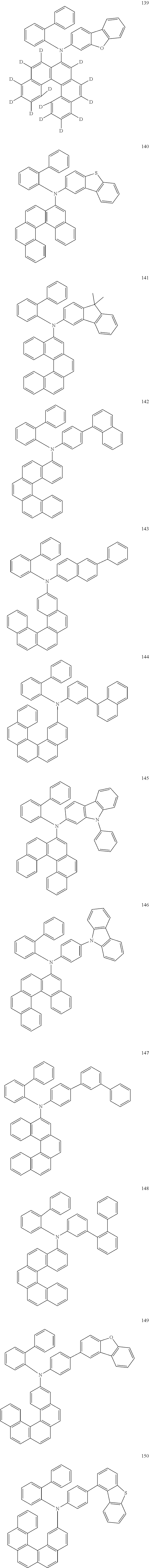

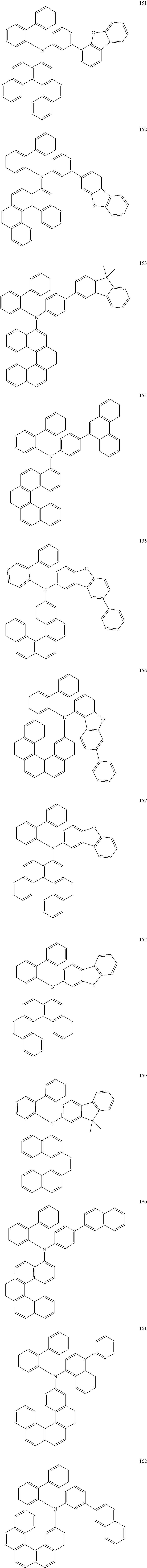

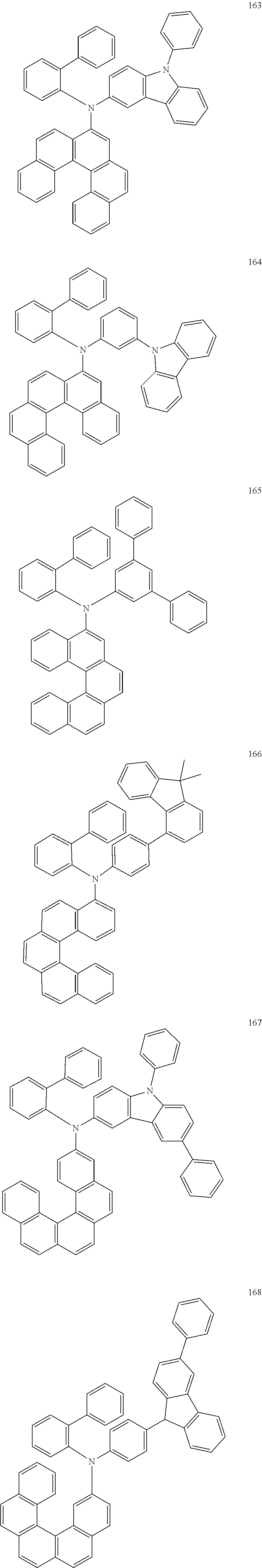

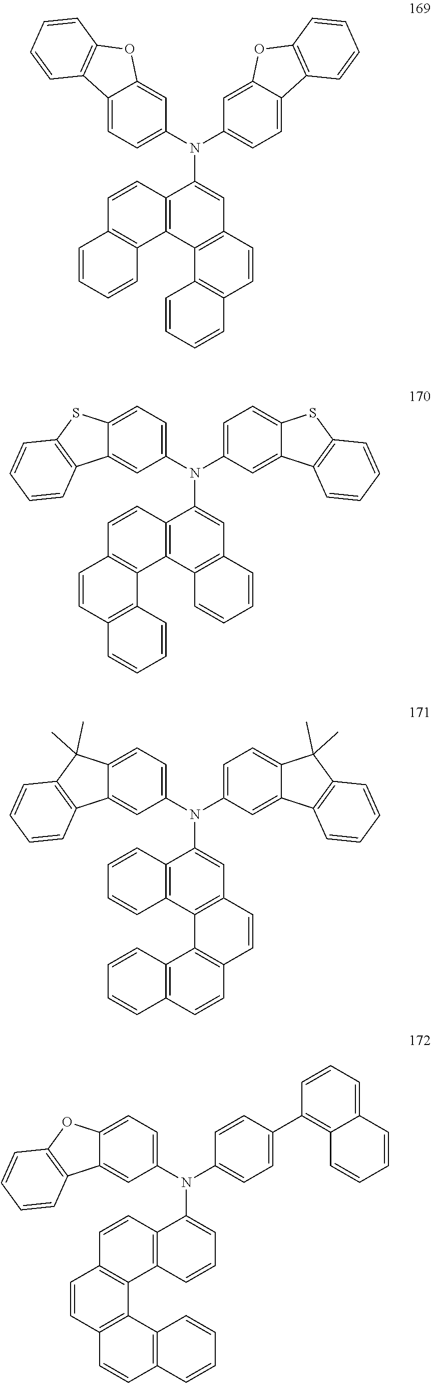 [SMILES: c1ccc2c(c1)ccc1cc(N(c3ccc4c(c3)oc3ccccc34)c3ccc4c(c3)oc3ccccc34)c3ccc4ccccc4c3c12 ; c1ccc2c(c1)ccc1ccc3c(N(c4ccc5sc6ccccc6c5c4)c4ccc5sc6ccccc6c5c4)cc4ccccc4c3c12 ; CC1(C)c2ccccc2-c2cc(N(c3ccc4c(c3)-c3ccccc3C4(C)C)c3cc4ccc5ccc6ccccc6c5c4c4ccccc34)ccc21 ; c1ccc2c(-c3ccc(N(c4ccc5oc6ccccc6c5c4)c4cccc5c4ccc4ccc6ccc7ccccc7c6c45)cc3)cccc2c1]

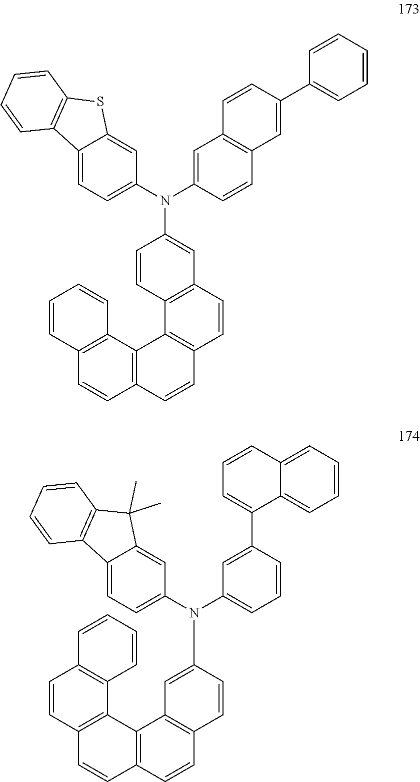 [SMILES: c1ccc(-c2ccc3cc(N(c4ccc5c(ccc6ccc7ccc8ccccc8c7c65)c4)c4ccc5c(c4)sc4ccccc45)ccc3c2)cc1 ; CC1(C)c2ccccc2-c2ccc(N(c3cccc(-c4cccc5ccccc45)c3)c3ccc4ccc5ccc6ccc7ccccc7c6c5c4c3)cc21]

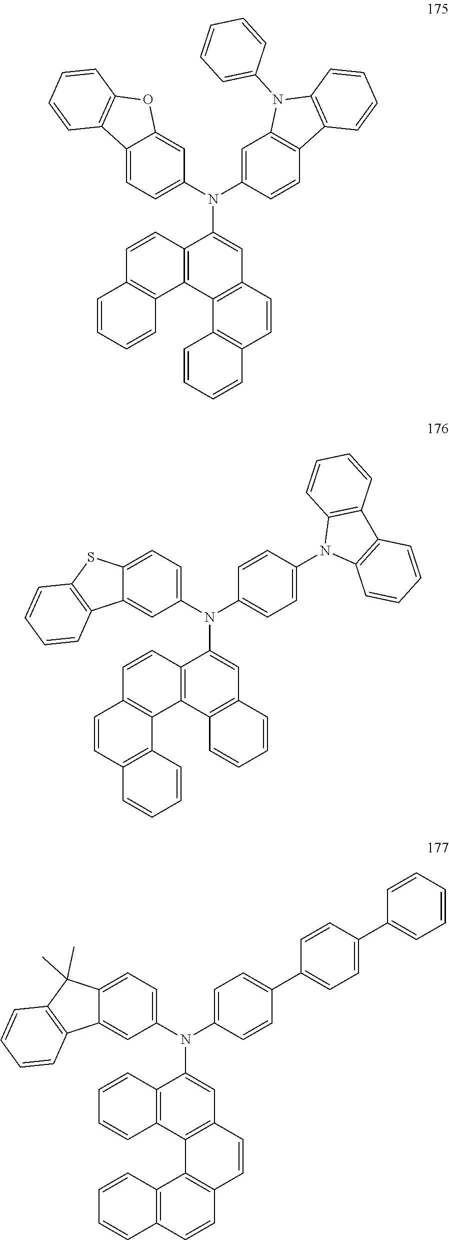 [SMILES: c1ccc(-n2c3ccccc3c3ccc(N(c4ccc5c(c4)oc4ccccc45)c4cc5ccc6ccccc6c5c5c4ccc4ccccc45)cc32)cc1 ; c1ccc2c(c1)ccc1ccc3c(N(c4ccc(-n5c6ccccc6c6ccccc65)cc4)c4ccc5sc6ccccc6c5c4)cc4ccccc4c3c12 ; CC1(C)c2ccccc2-c2cc(N(c3ccc(-c4ccc(-c5ccccc5)cc4)cc3)c3cc4ccc5ccc6ccccc6c5c4c4ccccc34)ccc21]

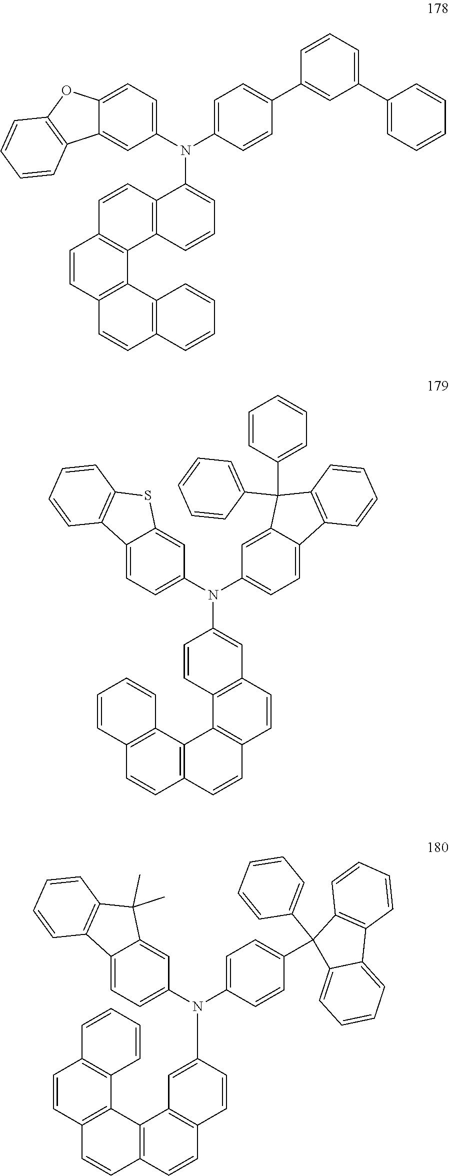 [SMILES: c1ccc(-c2cccc(-c3ccc(N(c4ccc5oc6ccccc6c5c4)c4cccc5c4ccc4ccc6ccc7ccccc7c6c45)cc3)c2)cc1 ; c1ccc(C2(c3ccccc3)c3ccccc3-c3ccc(N(c4ccc5c(ccc6ccc7ccc8ccccc8c7c65)c4)c4ccc5c(c4)sc4ccccc45)cc32)cc1 ; CC1(C)c2ccccc2-c2ccc(N(c3ccc(C4(c5ccccc5)c5ccccc5-c5ccccc54)cc3)c3ccc4ccc5ccc6ccc7ccccc7c6c5c4c3)cc21]

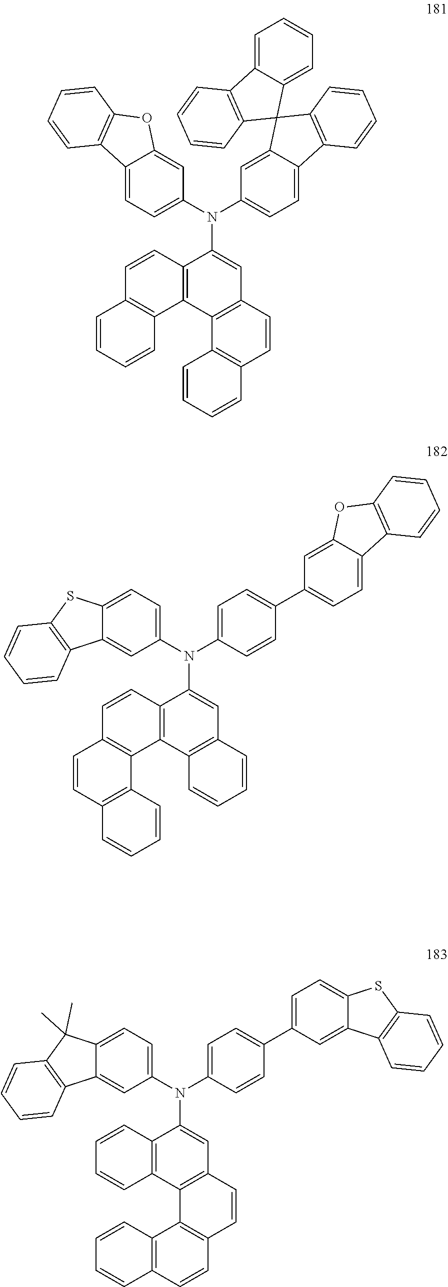 [SMILES: c1ccc2c(c1)-c1ccccc1C21c2ccccc2-c2ccc(N(c3ccc4c(c3)oc3ccccc34)c3cc4ccc5ccccc5c4c4c3ccc3ccccc34)cc21 ; c1ccc2c(c1)ccc1ccc3c(N(c4ccc(-c5ccc6c(c5)oc5ccccc56)cc4)c4ccc5sc6ccccc6c5c4)cc4ccccc4c3c12 ; CC1(C)c2ccccc2-c2cc(N(c3ccc(-c4ccc5sc6ccccc6c5c4)cc3)c3cc4ccc5ccc6ccccc6c5c4c4ccccc34)ccc21]

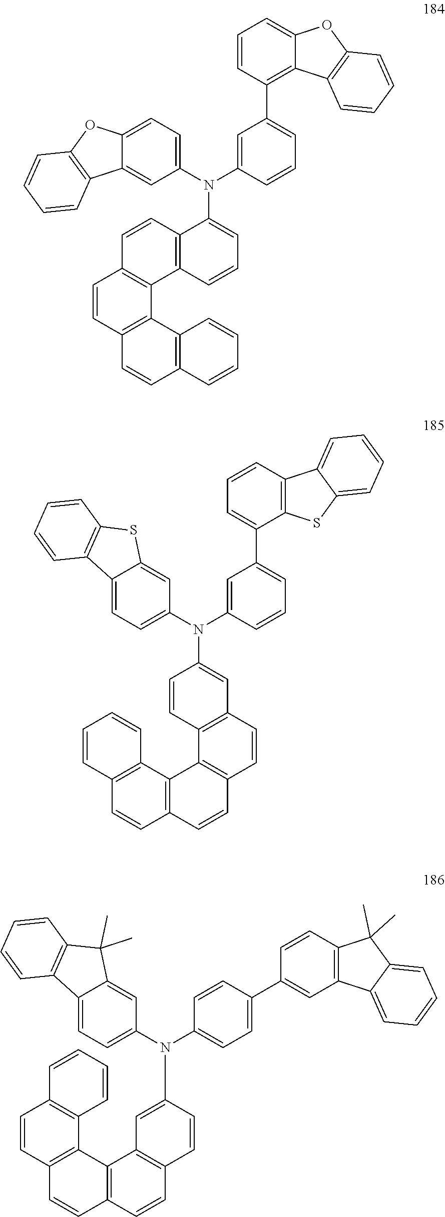 [SMILES: c1cc(-c2cccc3oc4ccccc4c23)cc(N(c2ccc3oc4ccccc4c3c2)c2cccc3c2ccc2ccc4ccc5ccccc5c4c23)c1 ; c1cc(-c2cccc3c2sc2ccccc23)cc(N(c2ccc3c(ccc4ccc5ccc6ccccc6c5c43)c2)c2ccc3c(c2)sc2ccccc23)c1 ; CC1(C)c2ccccc2-c2cc(-c3ccc(N(c4ccc5c(c4)C(C)(C)c4ccccc4-5)c4ccc5ccc6ccc7ccc8ccccc8c7c6c5c4)cc3)ccc21]

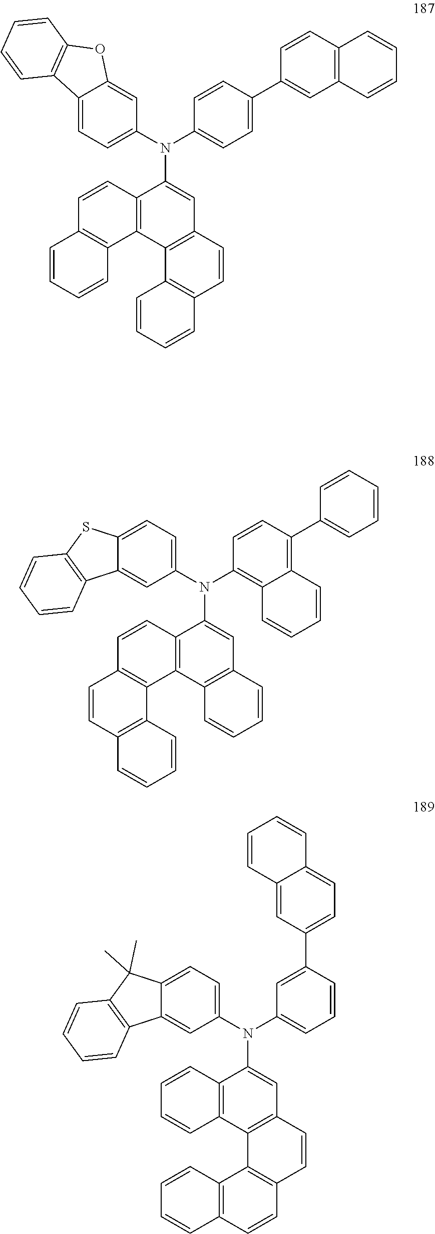 [SMILES: c1ccc2cc(-c3ccc(N(c4ccc5c(c4)oc4ccccc45)c4cc5ccc6ccccc6c5c5c4ccc4ccccc45)cc3)ccc2c1 ; c1ccc(-c2ccc(N(c3ccc4sc5ccccc5c4c3)c3cc4ccccc4c4c3ccc3ccc5ccccc5c34)c3ccccc23)cc1 ; CC1(C)c2ccccc2-c2cc(N(c3cccc(-c4ccc5ccccc5c4)c3)c3cc4ccc5ccc6ccccc6c5c4c4ccccc34)ccc21]

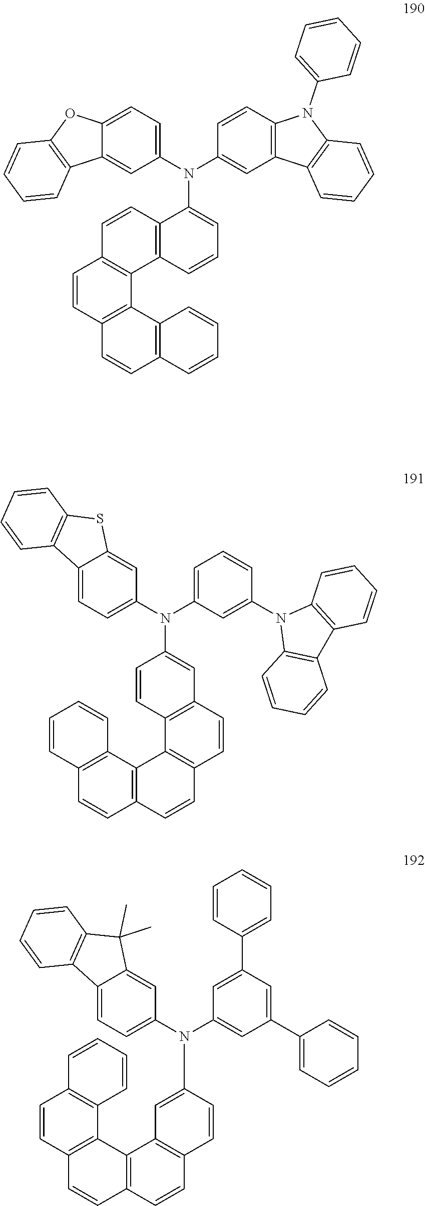 [SMILES: c1ccc(-n2c3ccccc3c3cc(N(c4ccc5oc6ccccc6c5c4)c4cccc5c4ccc4ccc6ccc7ccccc7c6c45)ccc32)cc1 ; c1cc(N(c2ccc3c(ccc4ccc5ccc6ccccc6c5c43)c2)c2ccc3c(c2)sc2ccccc23)cc(-n2c3ccccc3c3ccccc32)c1 ; CC1(C)c2ccccc2-c2ccc(N(c3cc(-c4ccccc4)cc(-c4ccccc4)c3)c3ccc4ccc5ccc6ccc7ccccc7c6c5c4c3)cc21]

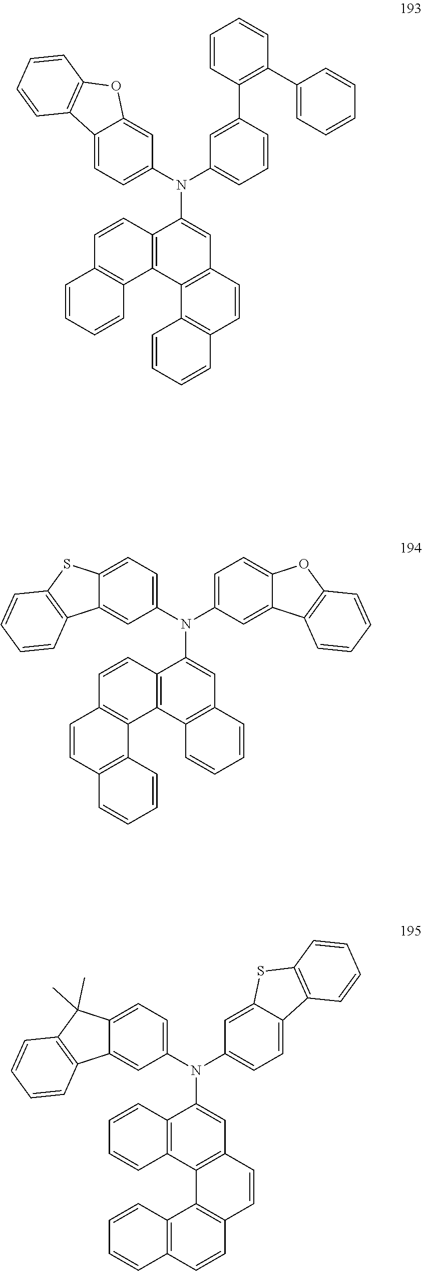 [SMILES: c1ccc(-c2ccccc2-c2cccc(N(c3ccc4c(c3)oc3ccccc34)c3cc4ccc5ccccc5c4c4c3ccc3ccccc34)c2)cc1 ; c1ccc2c(c1)ccc1ccc3c(N(c4ccc5oc6ccccc6c5c4)c4ccc5sc6ccccc6c5c4)cc4ccccc4c3c12 ; CC1(C)c2ccccc2-c2cc(N(c3ccc4c(c3)sc3ccccc34)c3cc4ccc5ccc6ccccc6c5c4c4ccccc34)ccc21]

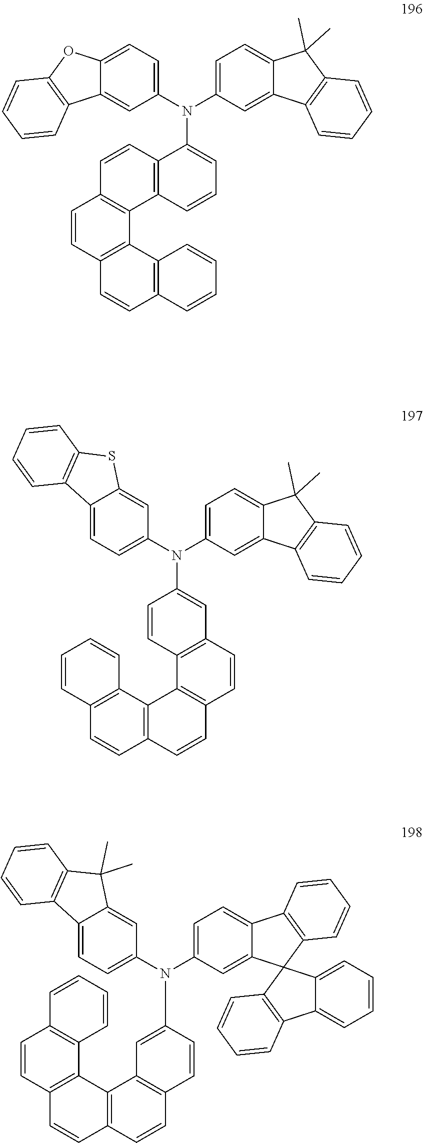 [SMILES: CC1(C)c2ccccc2-c2cc(N(c3ccc4oc5ccccc5c4c3)c3cccc4c3ccc3ccc5ccc6ccccc6c5c34)ccc21 ; CC1(C)c2ccccc2-c2cc(N(c3ccc4c(ccc5ccc6ccc7ccccc7c6c54)c3)c3ccc4c(c3)sc3ccccc34)ccc21 ; CC1(C)c2ccccc2-c2ccc(N(c3ccc4c(c3)C3(c5ccccc5-c5ccccc53)c3ccccc3-4)c3ccc4ccc5ccc6ccc7ccccc7c6c5c4c3)cc21]

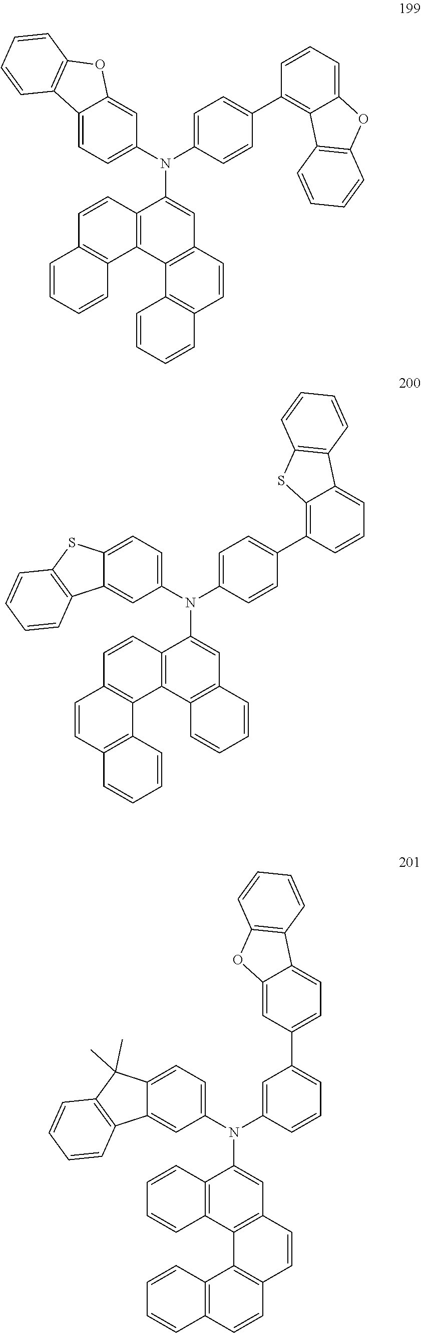 [SMILES: c1ccc2c(c1)ccc1cc(N(c3ccc(-c4cccc5oc6ccccc6c45)cc3)c3ccc4c(c3)oc3ccccc34)c3ccc4ccccc4c3c12 ; c1ccc2c(c1)ccc1ccc3c(N(c4ccc(-c5cccc6c5sc5ccccc56)cc4)c4ccc5sc6ccccc6c5c4)cc4ccccc4c3c12 ; CC1(C)c2ccccc2-c2cc(N(c3cccc(-c4ccc5c(c4)oc4ccccc45)c3)c3cc4ccc5ccc6ccccc6c5c4c4ccccc34)ccc21]

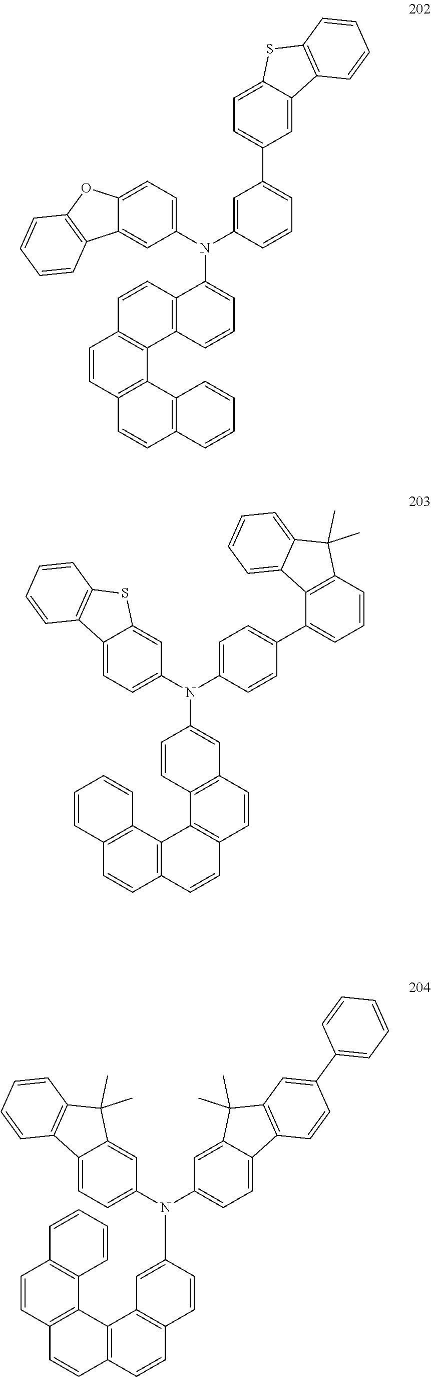 [SMILES: c1cc(-c2ccc3sc4ccccc4c3c2)cc(N(c2ccc3oc4ccccc4c3c2)c2cccc3c2ccc2ccc4ccc5ccccc5c4c23)c1 ; CC1(C)c2ccccc2-c2c(-c3ccc(N(c4ccc5c(ccc6ccc7ccc8ccccc8c7c65)c4)c4ccc5c(c4)sc4ccccc45)cc3)cccc21 ; CC1(C)c2ccccc2-c2ccc(N(c3ccc4c(c3)C(C)(C)c3cc(-c5ccccc5)ccc3-4)c3ccc4ccc5ccc6ccc7ccccc7c6c5c4c3)cc21]

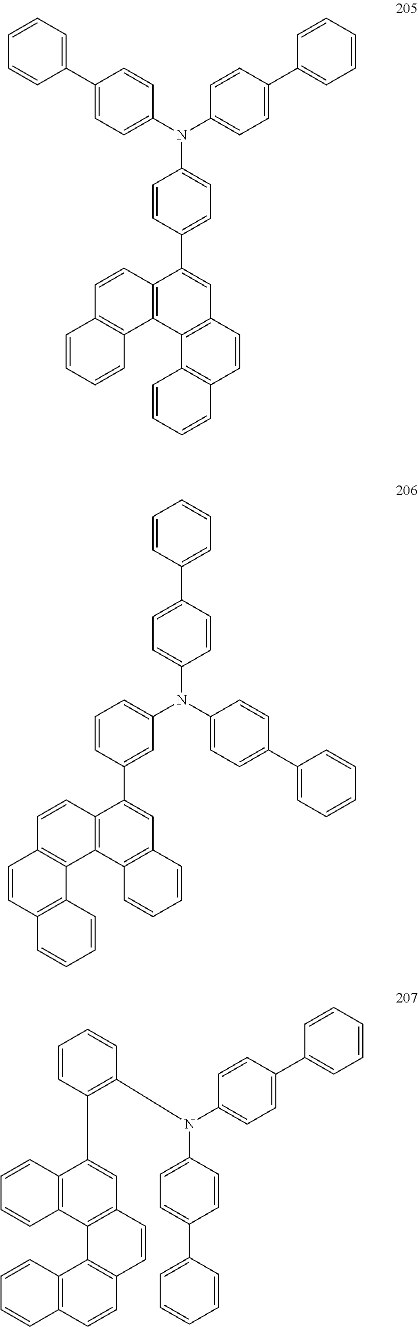 [SMILES: c1ccc(-c2ccc(N(c3ccc(-c4ccccc4)cc3)c3ccc(-c4cc5ccc6ccccc6c5c5c4ccc4ccccc45)cc3)cc2)cc1 ; c1ccc(-c2ccc(N(c3ccc(-c4ccccc4)cc3)c3cccc(-c4cc5ccccc5c5c4ccc4ccc6ccccc6c45)c3)cc2)cc1 ; c1ccc(-c2ccc(N(c3ccc(-c4ccccc4)cc3)c3ccccc3-c3cc4ccc5ccc6ccccc6c5c4c4ccccc34)cc2)cc1]

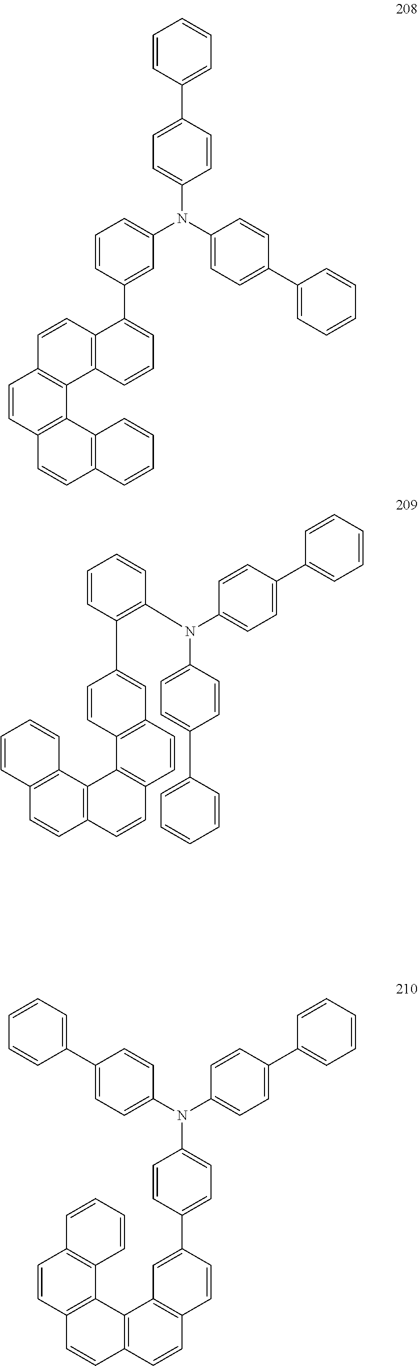 [SMILES: c1ccc(-c2ccc(N(c3ccc(-c4ccccc4)cc3)c3cccc(-c4cccc5c4ccc4ccc6ccc7ccccc7c6c45)c3)cc2)cc1 ; c1ccc(-c2ccc(N(c3ccc(-c4ccccc4)cc3)c3ccccc3-c3ccc4c(ccc5ccc6ccc7ccccc7c6c54)c3)cc2)cc1 ; c1ccc(-c2ccc(N(c3ccc(-c4ccccc4)cc3)c3ccc(-c4ccc5ccc6ccc7ccc8ccccc8c7c6c5c4)cc3)cc2)cc1]

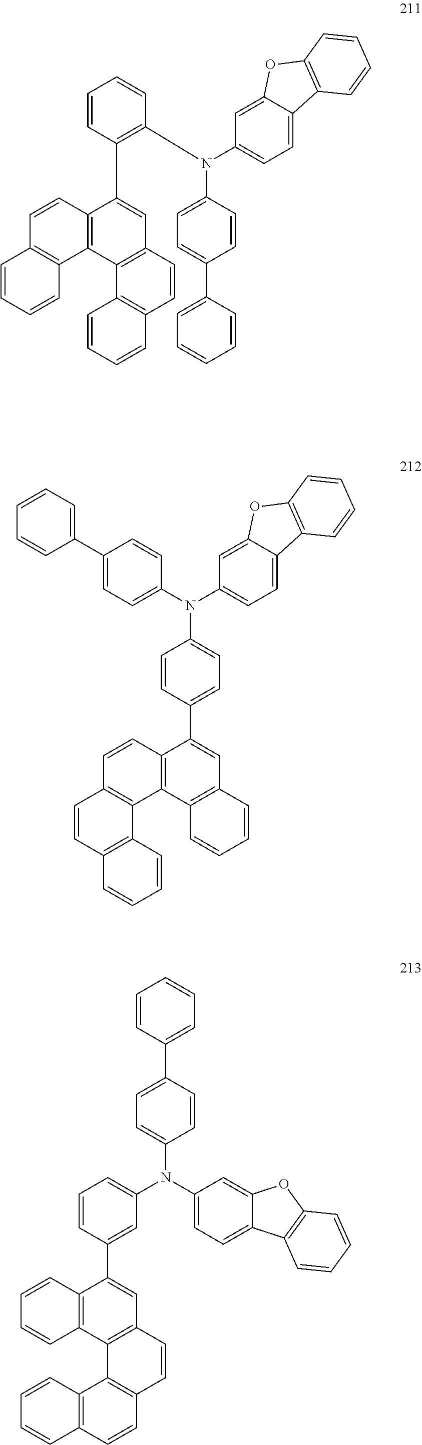 [SMILES: c1ccc(-c2ccc(N(c3ccc4c(c3)oc3ccccc34)c3ccccc3-c3cc4ccc5ccccc5c4c4c3ccc3ccccc34)cc2)cc1 ; c1ccc(-c2ccc(N(c3ccc(-c4cc5ccccc5c5c4ccc4ccc6ccccc6c45)cc3)c3ccc4c(c3)oc3ccccc34)cc2)cc1 ; c1ccc(-c2ccc(N(c3cccc(-c4cc5ccc6ccc7ccccc7c6c5c5ccccc45)c3)c3ccc4c(c3)oc3ccccc34)cc2)cc1]

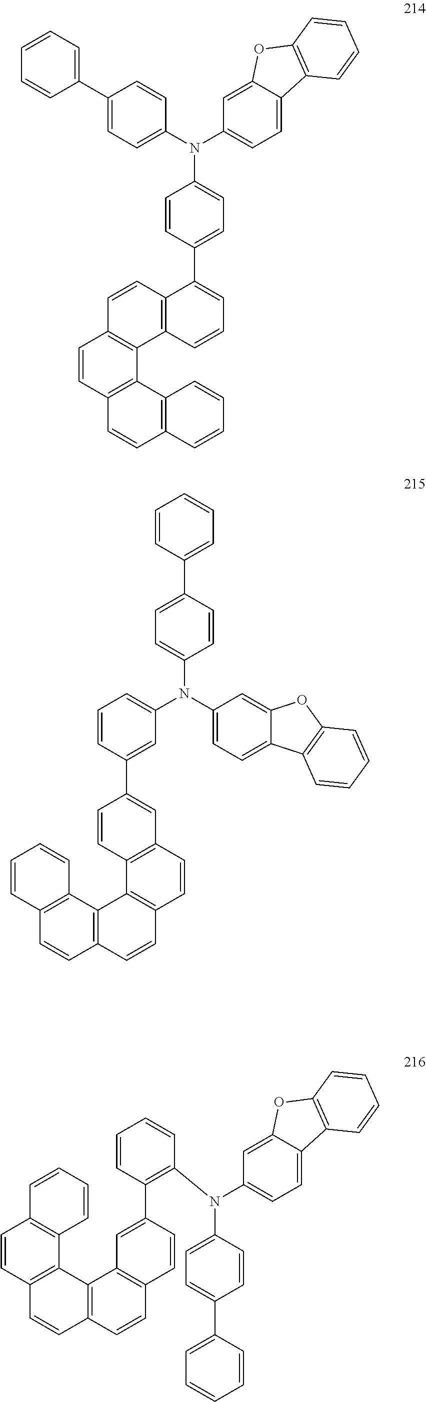 [SMILES: c1ccc(-c2ccc(N(c3ccc(-c4cccc5c4ccc4ccc6ccc7ccccc7c6c45)cc3)c3ccc4c(c3)oc3ccccc34)cc2)cc1 ; c1ccc(-c2ccc(N(c3cccc(-c4ccc5c(ccc6ccc7ccc8ccccc8c7c65)c4)c3)c3ccc4c(c3)oc3ccccc34)cc2)cc1 ; c1ccc(-c2ccc(N(c3ccc4c(c3)oc3ccccc34)c3ccccc3-c3ccc4ccc5ccc6ccc7ccccc7c6c5c4c3)cc2)cc1]

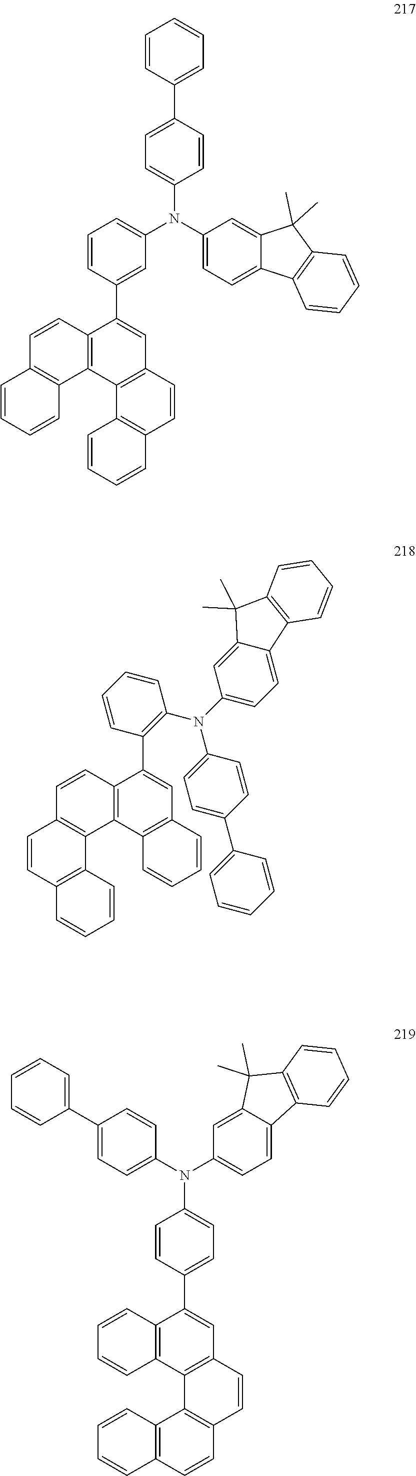 [SMILES: CC1(C)c2ccccc2-c2ccc(N(c3ccc(-c4ccccc4)cc3)c3cccc(-c4cc5ccc6ccccc6c5c5c4ccc4ccccc45)c3)cc21 ; CC1(C)c2ccccc2-c2ccc(N(c3ccc(-c4ccccc4)cc3)c3ccccc3-c3cc4ccccc4c4c3ccc3ccc5ccccc5c34)cc21 ; CC1(C)c2ccccc2-c2ccc(N(c3ccc(-c4ccccc4)cc3)c3ccc(-c4cc5ccc6ccc7ccccc7c6c5c5ccccc45)cc3)cc21]

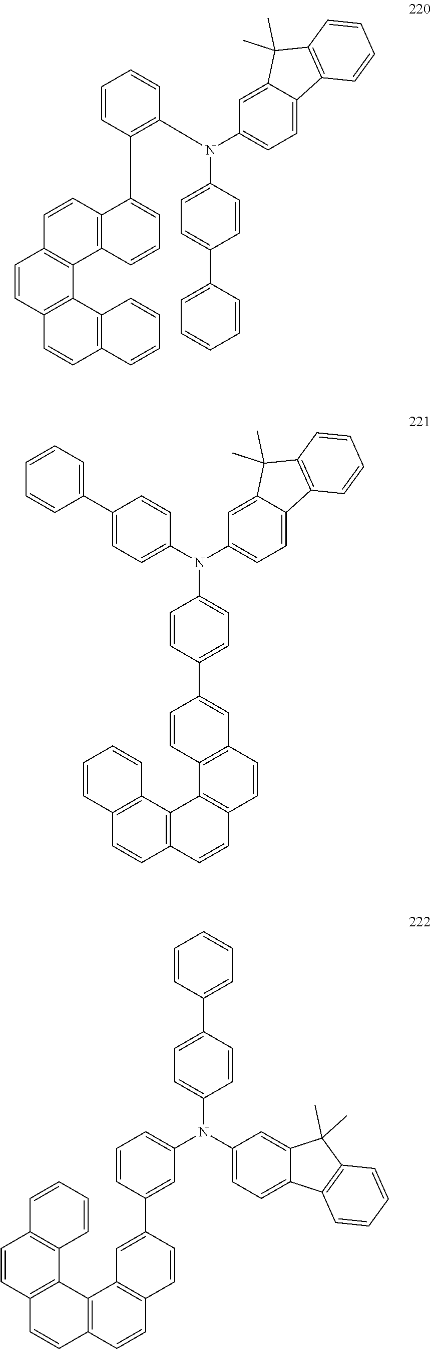 [SMILES: CC1(C)c2ccccc2-c2ccc(N(c3ccc(-c4ccccc4)cc3)c3ccccc3-c3cccc4c3ccc3ccc5ccc6ccccc6c5c34)cc21 ; CC1(C)c2ccccc2-c2ccc(N(c3ccc(-c4ccccc4)cc3)c3ccc(-c4ccc5c(ccc6ccc7ccc8ccccc8c7c65)c4)cc3)cc21 ; CC1(C)c2ccccc2-c2ccc(N(c3ccc(-c4ccccc4)cc3)c3cccc(-c4ccc5ccc6ccc7ccc8ccccc8c7c6c5c4)c3)cc21]

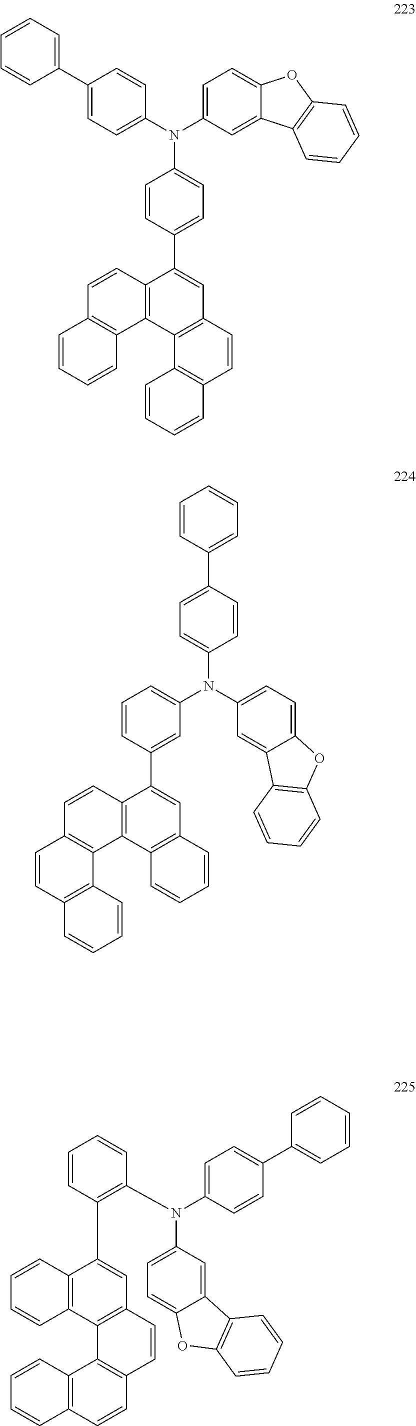 [SMILES: c1ccc(-c2ccc(N(c3ccc(-c4cc5ccc6ccccc6c5c5c4ccc4ccccc45)cc3)c3ccc4oc5ccccc5c4c3)cc2)cc1 ; c1ccc(-c2ccc(N(c3cccc(-c4cc5ccccc5c5c4ccc4ccc6ccccc6c45)c3)c3ccc4oc5ccccc5c4c3)cc2)cc1 ; c1ccc(-c2ccc(N(c3ccc4oc5ccccc5c4c3)c3ccccc3-c3cc4ccc5ccc6ccccc6c5c4c4ccccc34)cc2)cc1]

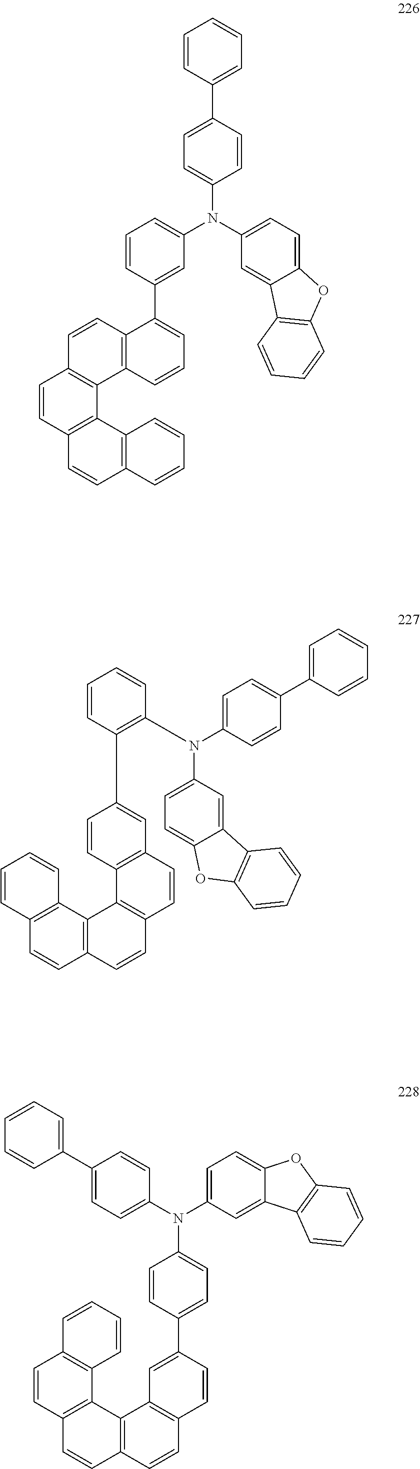 [SMILES: c1ccc(-c2ccc(N(c3cccc(-c4cccc5c4ccc4ccc6ccc7ccccc7c6c45)c3)c3ccc4oc5ccccc5c4c3)cc2)cc1 ; c1ccc(-c2ccc(N(c3ccc4oc5ccccc5c4c3)c3ccccc3-c3ccc4c(ccc5ccc6ccc7ccccc7c6c54)c3)cc2)cc1 ; c1ccc(-c2ccc(N(c3ccc(-c4ccc5ccc6ccc7ccc8ccccc8c7c6c5c4)cc3)c3ccc4oc5ccccc5c4c3)cc2)cc1]

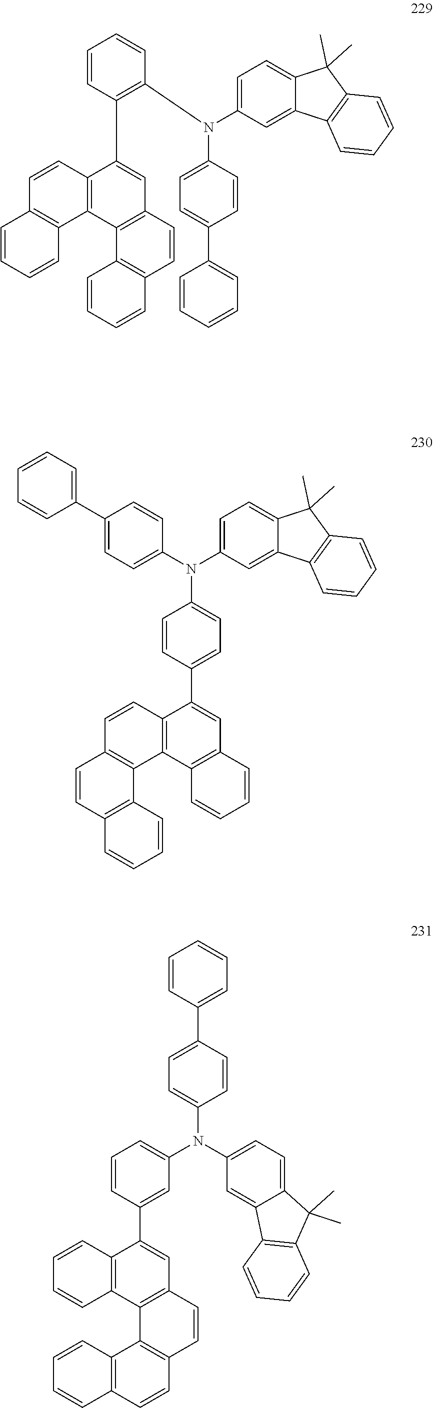 [SMILES: CC1(C)c2ccccc2-c2cc(N(c3ccc(-c4ccccc4)cc3)c3ccccc3-c3cc4ccc5ccccc5c4c4c3ccc3ccccc34)ccc21 ; CC1(C)c2ccccc2-c2cc(N(c3ccc(-c4ccccc4)cc3)c3ccc(-c4cc5ccccc5c5c4ccc4ccc6ccccc6c45)cc3)ccc21 ; CC1(C)c2ccccc2-c2cc(N(c3ccc(-c4ccccc4)cc3)c3cccc(-c4cc5ccc6ccc7ccccc7c6c5c5ccccc45)c3)ccc21]

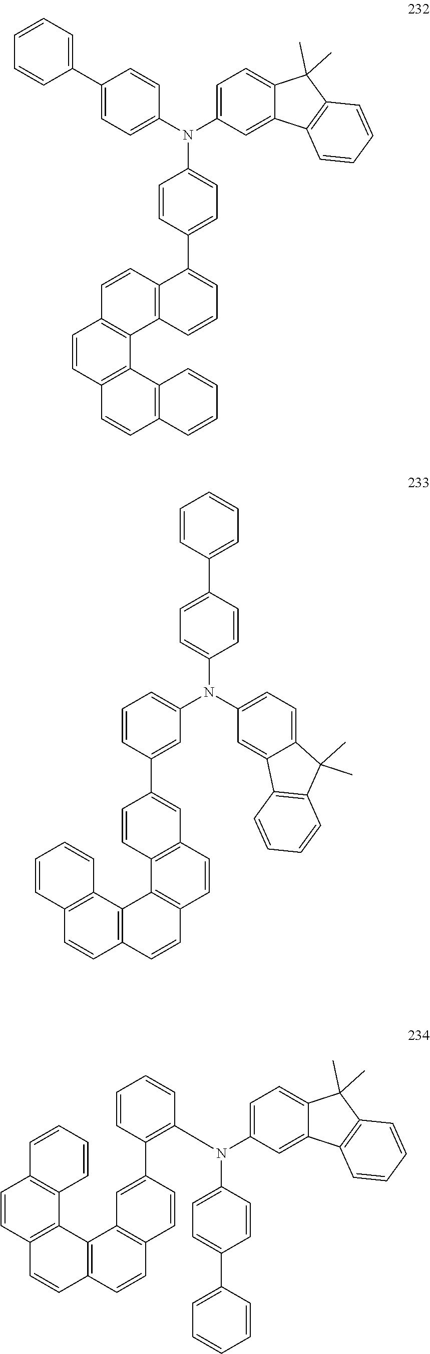 [SMILES: CC1(C)c2ccccc2-c2cc(N(c3ccc(-c4ccccc4)cc3)c3ccc(-c4cccc5c4ccc4ccc6ccc7ccccc7c6c45)cc3)ccc21 ; CC1(C)c2ccccc2-c2cc(N(c3ccc(-c4ccccc4)cc3)c3cccc(-c4ccc5c(ccc6ccc7ccc8ccccc8c7c65)c4)c3)ccc21 ; CC1(C)c2ccccc2-c2cc(N(c3ccc(-c4ccccc4)cc3)c3ccccc3-c3ccc4ccc5ccc6ccc7ccccc7c6c5c4c3)ccc21]

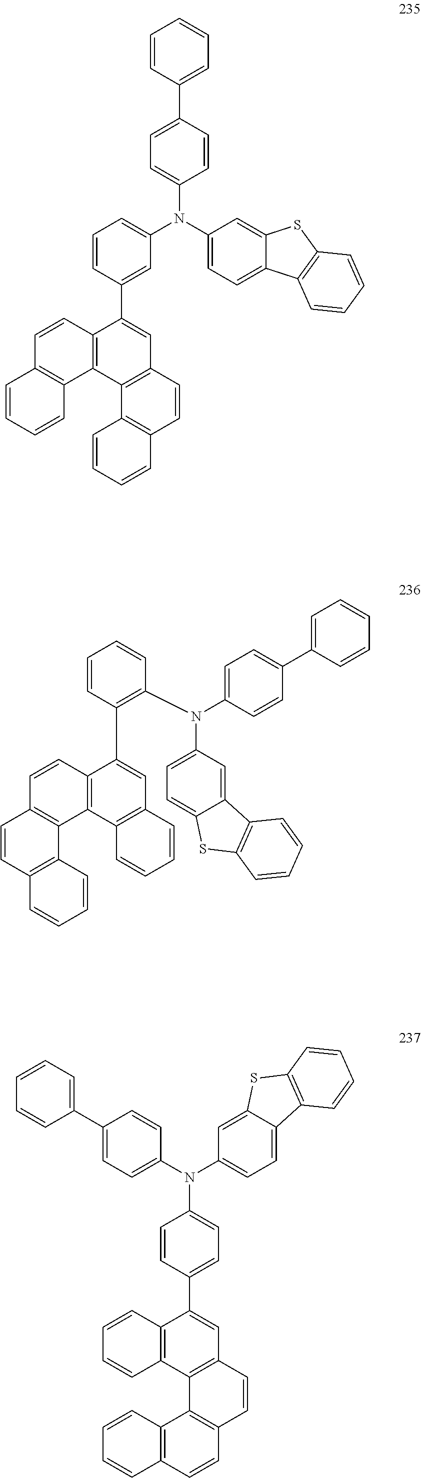 [SMILES: c1ccc(-c2ccc(N(c3cccc(-c4cc5ccc6ccccc6c5c5c4ccc4ccccc45)c3)c3ccc4c(c3)sc3ccccc34)cc2)cc1 ; c1ccc(-c2ccc(N(c3ccc4sc5ccccc5c4c3)c3ccccc3-c3cc4ccccc4c4c3ccc3ccc5ccccc5c34)cc2)cc1 ; c1ccc(-c2ccc(N(c3ccc(-c4cc5ccc6ccc7ccccc7c6c5c5ccccc45)cc3)c3ccc4c(c3)sc3ccccc34)cc2)cc1]

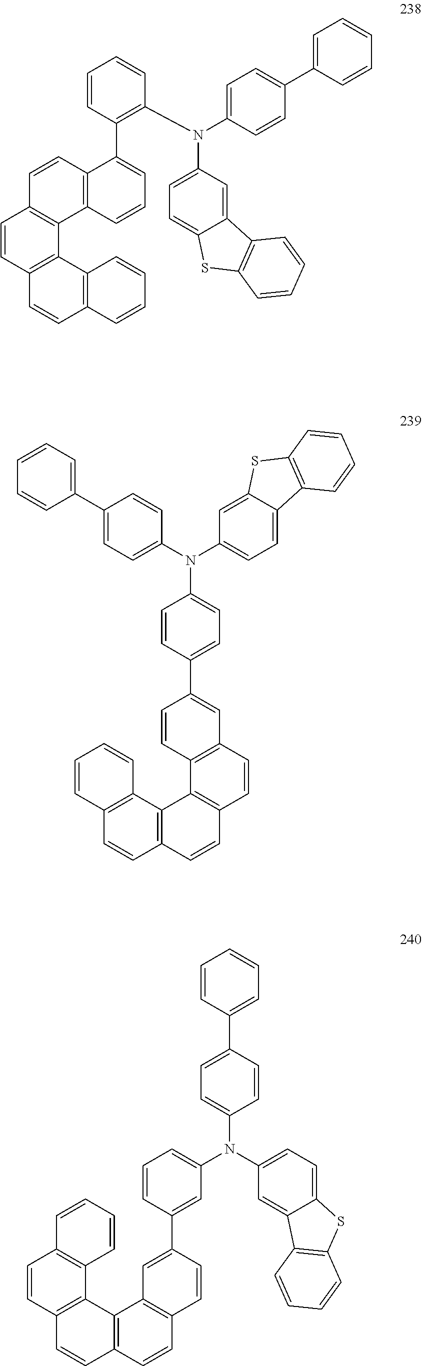 [SMILES: c1ccc(-c2ccc(N(c3ccc4sc5ccccc5c4c3)c3ccccc3-c3cccc4c3ccc3ccc5ccc6ccccc6c5c34)cc2)cc1 ; c1ccc(-c2ccc(N(c3ccc(-c4ccc5c(ccc6ccc7ccc8ccccc8c7c65)c4)cc3)c3ccc4c(c3)sc3ccccc34)cc2)cc1 ; c1ccc(-c2ccc(N(c3cccc(-c4ccc5ccc6ccc7ccc8ccccc8c7c6c5c4)c3)c3ccc4sc5ccccc5c4c3)cc2)cc1]

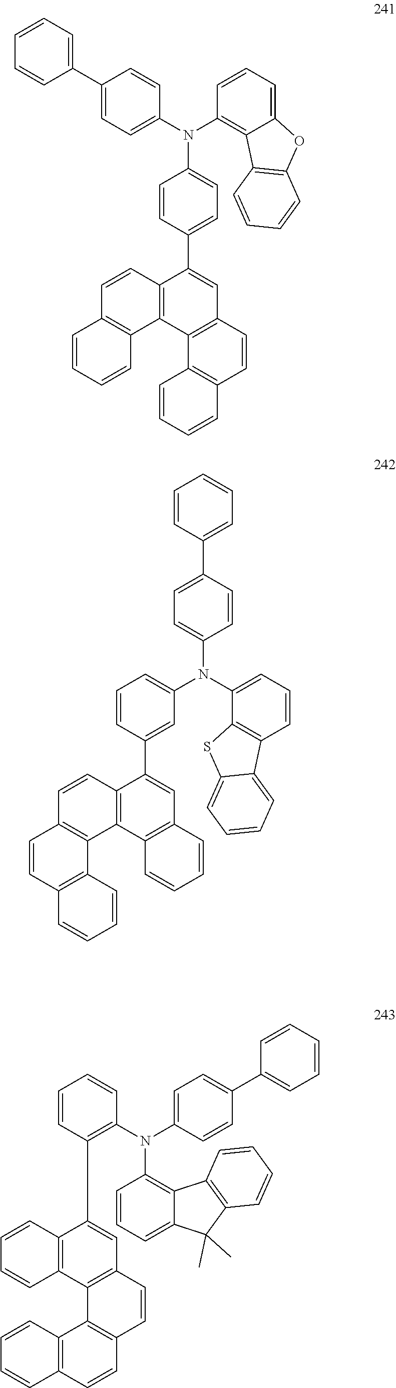

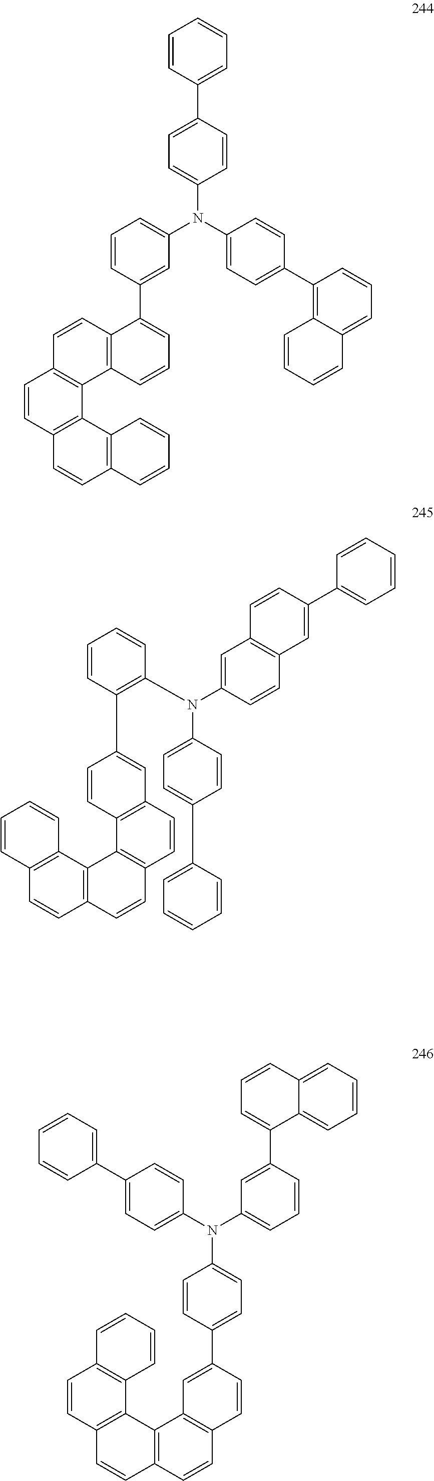 [SMILES: c1ccc(-c2ccc(N(c3ccc(-c4cccc5ccccc45)cc3)c3cccc(-c4cccc5c4ccc4ccc6ccc7ccccc7c6c45)c3)cc2)cc1 ; c1ccc(-c2ccc(N(c3ccc4cc(-c5ccccc5)ccc4c3)c3ccccc3-c3ccc4c(ccc5ccc6ccc7ccccc7c6c54)c3)cc2)cc1 ; c1ccc(-c2ccc(N(c3ccc(-c4ccc5ccc6ccc7ccc8ccccc8c7c6c5c4)cc3)c3cccc(-c4cccc5ccccc45)c3)cc2)cc1]

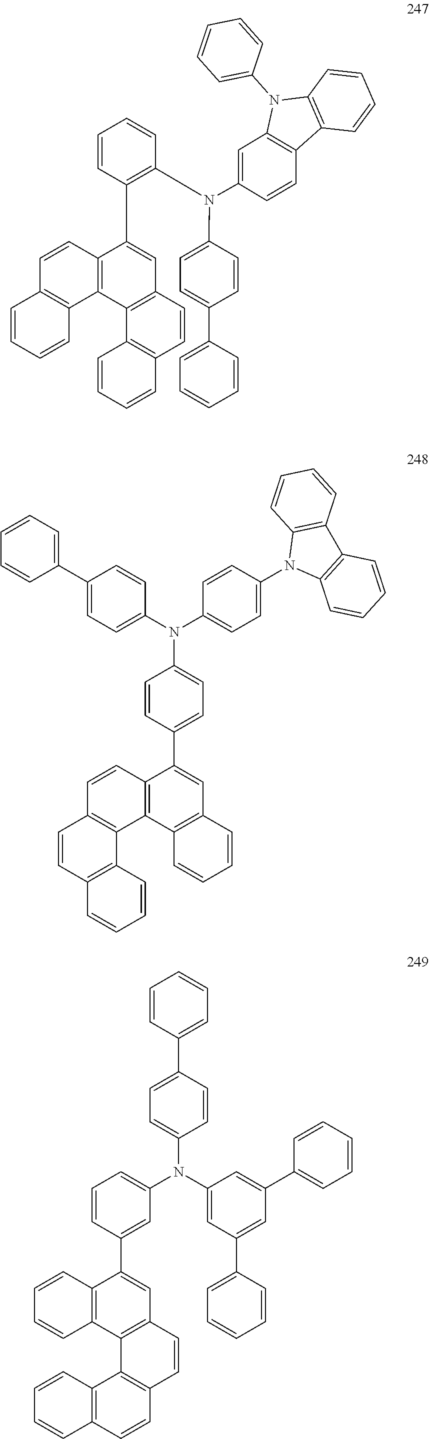 [SMILES: c1ccc(-c2ccc(N(c3ccc4c5ccccc5n(-c5ccccc5)c4c3)c3ccccc3-c3cc4ccc5ccccc5c4c4c3ccc3ccccc34)cc2)cc1 ; c1ccc(-c2ccc(N(c3ccc(-c4cc5ccccc5c5c4ccc4ccc6ccccc6c45)cc3)c3ccc(-n4c5ccccc5c5ccccc54)cc3)cc2)cc1 ; c1ccc(-c2ccc(N(c3cc(-c4ccccc4)cc(-c4ccccc4)c3)c3cccc(-c4cc5ccc6ccc7ccccc7c6c5c5ccccc45)c3)cc2)cc1]

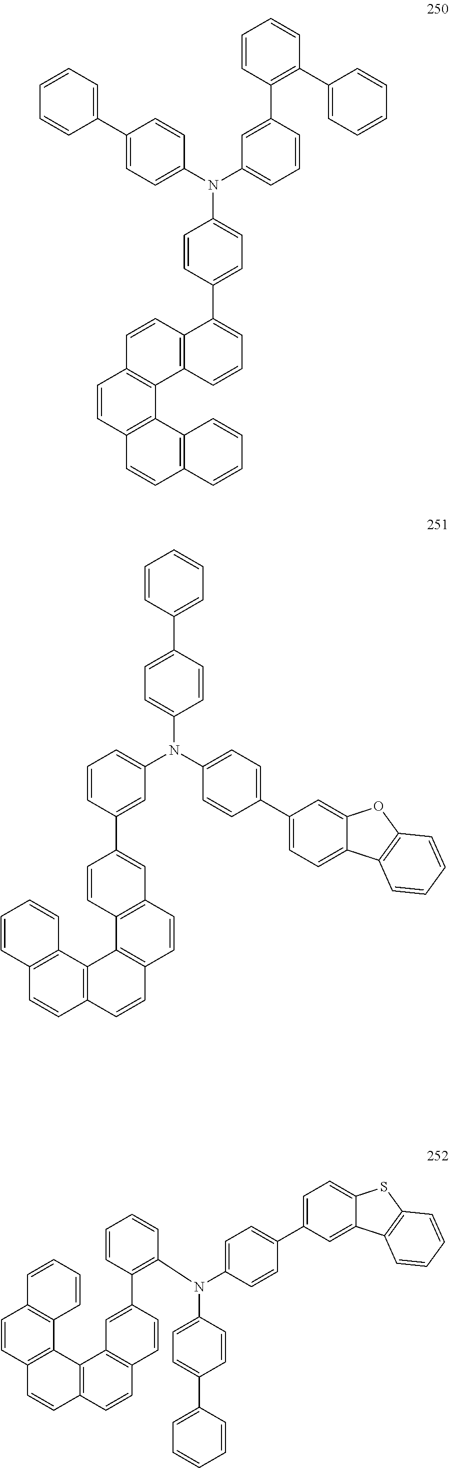 [SMILES: c1ccc(-c2ccc(N(c3ccc(-c4cccc5c4ccc4ccc6ccc7ccccc7c6c45)cc3)c3cccc(-c4ccccc4-c4ccccc4)c3)cc2)cc1 ; c1ccc(-c2ccc(N(c3ccc(-c4ccc5c(c4)oc4ccccc45)cc3)c3cccc(-c4ccc5c(ccc6ccc7ccc8ccccc8c7c65)c4)c3)cc2)cc1 ; c1ccc(-c2ccc(N(c3ccc(-c4ccc5sc6ccccc6c5c4)cc3)c3ccccc3-c3ccc4ccc5ccc6ccc7ccccc7c6c5c4c3)cc2)cc1]

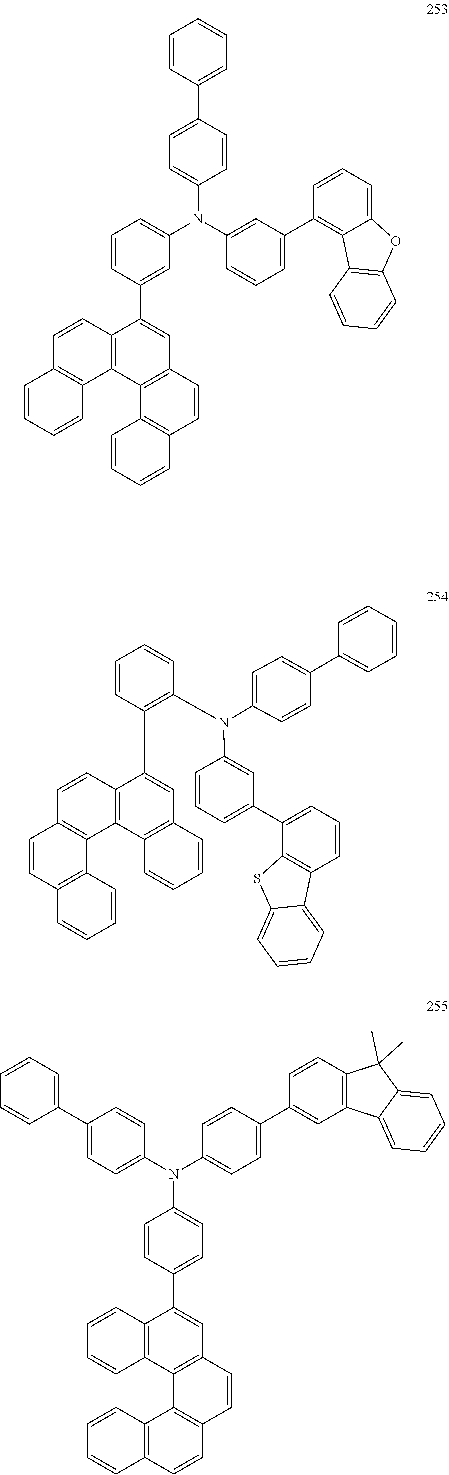 [SMILES: c1ccc(-c2ccc(N(c3cccc(-c4cc5ccc6ccccc6c5c5c4ccc4ccccc45)c3)c3cccc(-c4cccc5oc6ccccc6c45)c3)cc2)cc1 ; c1ccc(-c2ccc(N(c3cccc(-c4cccc5c4sc4ccccc45)c3)c3ccccc3-c3cc4ccccc4c4c3ccc3ccc5ccccc5c34)cc2)cc1 ; CC1(C)c2ccccc2-c2cc(-c3ccc(N(c4ccc(-c5ccccc5)cc4)c4ccc(-c5cc6ccc7ccc8ccccc8c7c6c6ccccc56)cc4)cc3)ccc21]

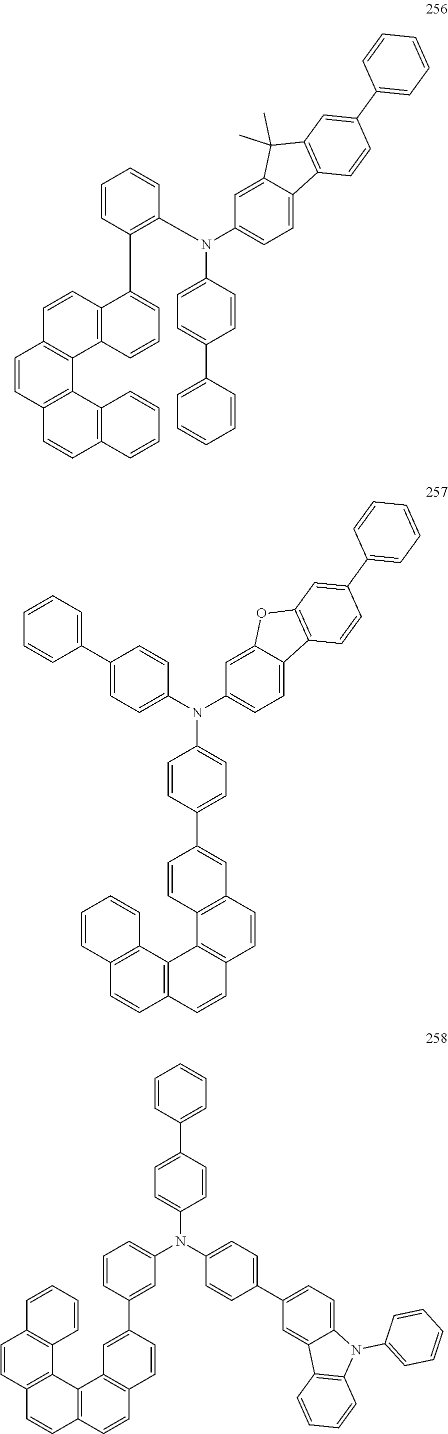 [SMILES: CC1(C)c2cc(-c3ccccc3)ccc2-c2ccc(N(c3ccc(-c4ccccc4)cc3)c3ccccc3-c3cccc4c3ccc3ccc5ccc6ccccc6c5c34)cc21 ; c1ccc(-c2ccc(N(c3ccc(-c4ccc5c(ccc6ccc7ccc8ccccc8c7c65)c4)cc3)c3ccc4c(c3)oc3cc(-c5ccccc5)ccc34)cc2)cc1 ; c1ccc(-c2ccc(N(c3ccc(-c4ccc5c(c4)c4ccccc4n5-c4ccccc4)cc3)c3cccc(-c4ccc5ccc6ccc7ccc8ccccc8c7c6c5c4)c3)cc2)cc1]

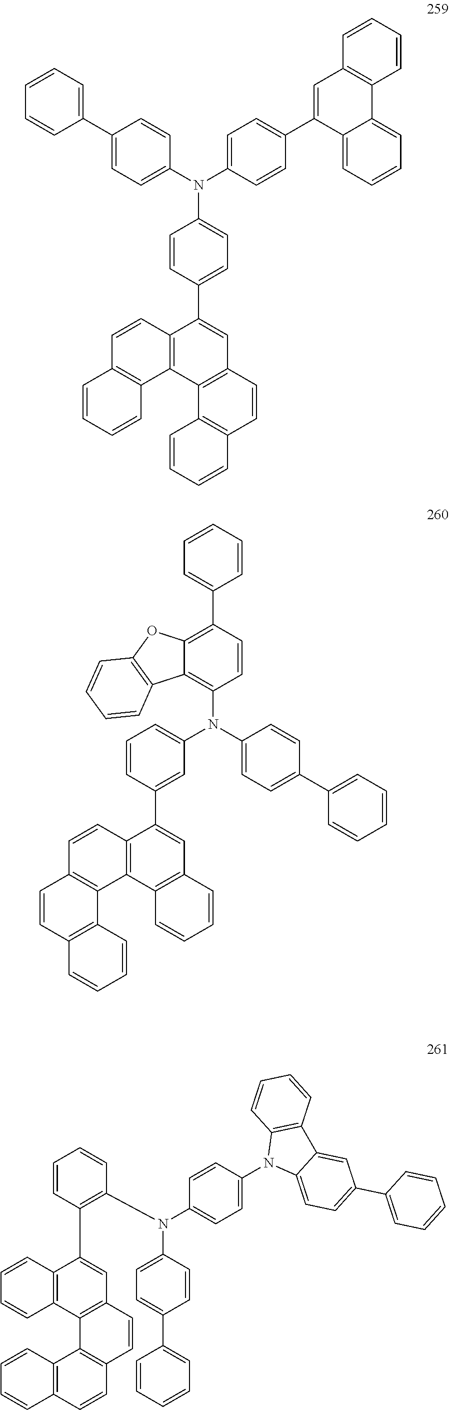

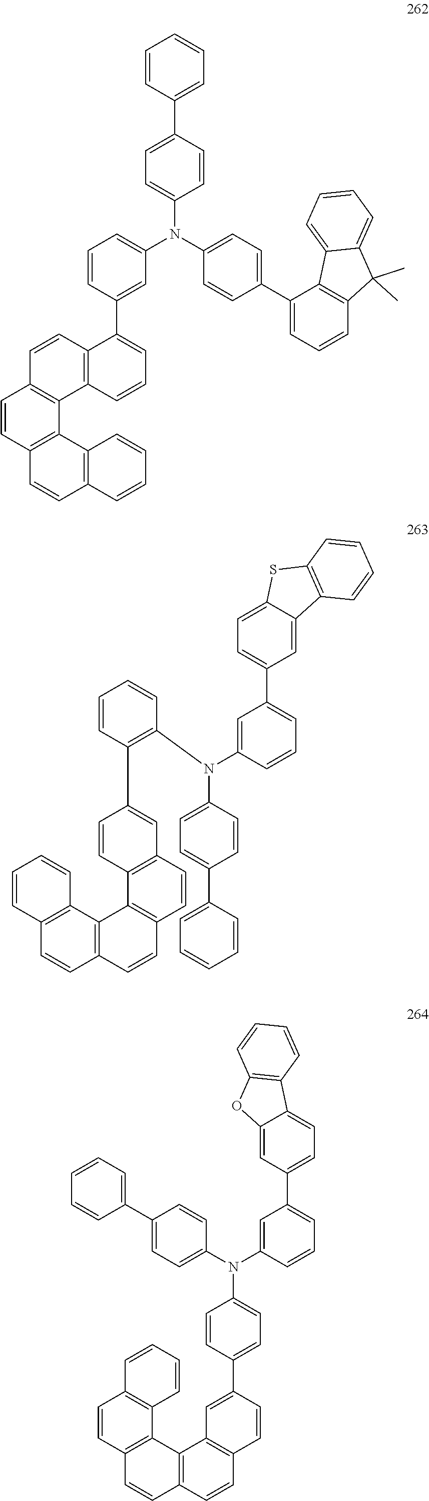 [SMILES: CC1(C)c2ccccc2-c2c(-c3ccc(N(c4ccc(-c5ccccc5)cc4)c4cccc(-c5cccc6c5ccc5ccc7ccc8ccccc8c7c56)c4)cc3)cccc21 ; c1ccc(-c2ccc(N(c3cccc(-c4ccc5sc6ccccc6c5c4)c3)c3ccccc3-c3ccc4c(ccc5ccc6ccc7ccccc7c6c54)c3)cc2)cc1 ; c1ccc(-c2ccc(N(c3ccc(-c4ccc5ccc6ccc7ccc8ccccc8c7c6c5c4)cc3)c3cccc(-c4ccc5c(c4)oc4ccccc45)c3)cc2)cc1]

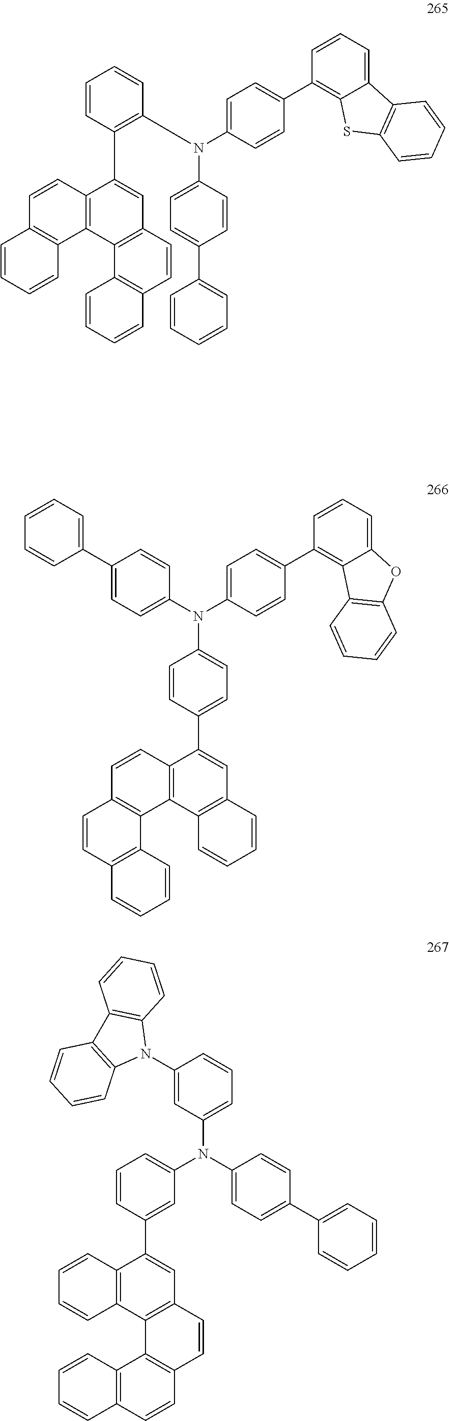

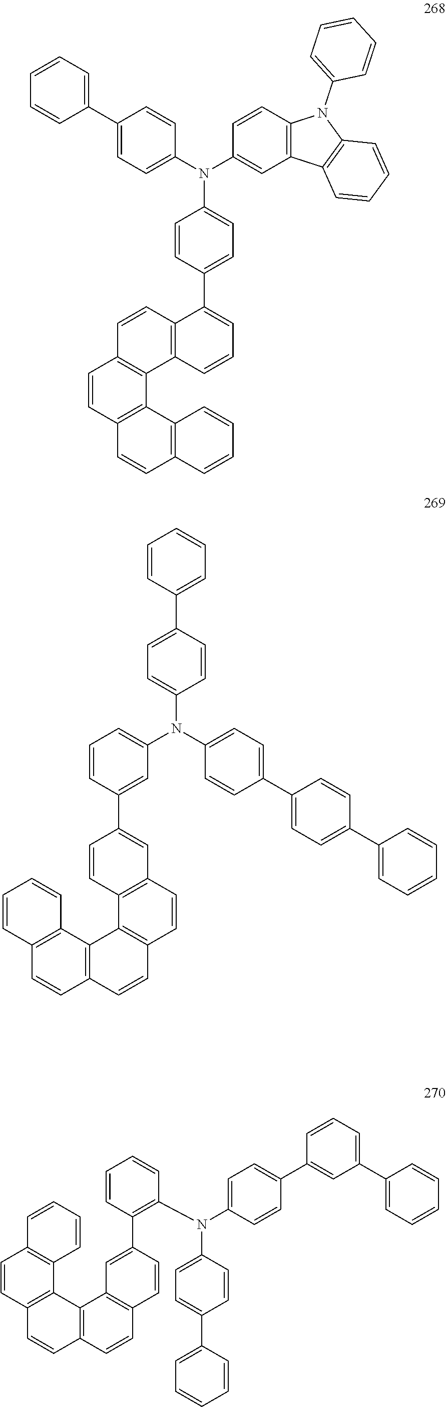 [SMILES: c1ccc(-c2ccc(N(c3ccc(-c4cccc5c4ccc4ccc6ccc7ccccc7c6c45)cc3)c3ccc4c(c3)c3ccccc3n4-c3ccccc3)cc2)cc1 ; c1ccc(-c2ccc(-c3ccc(N(c4ccc(-c5ccccc5)cc4)c4cccc(-c5ccc6c(ccc7ccc8ccc9ccccc9c8c76)c5)c4)cc3)cc2)cc1 ; c1ccc(-c2ccc(N(c3ccc(-c4cccc(-c5ccccc5)c4)cc3)c3ccccc3-c3ccc4ccc5ccc6ccc7ccccc7c6c5c4c3)cc2)cc1]

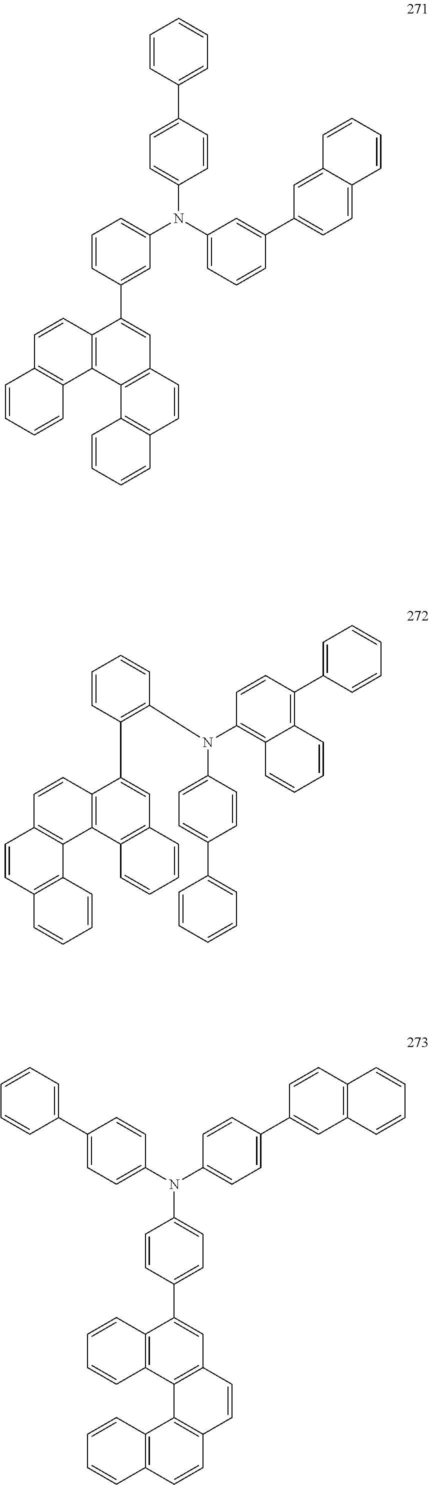 [SMILES: c1ccc(-c2ccc(N(c3cccc(-c4ccc5ccccc5c4)c3)c3cccc(-c4cc5ccc6ccccc6c5c5c4ccc4ccccc45)c3)cc2)cc1 ; c1ccc(-c2ccc(N(c3ccccc3-c3cc4ccccc4c4c3ccc3ccc5ccccc5c34)c3ccc(-c4ccccc4)c4ccccc34)cc2)cc1 ; c1ccc(-c2ccc(N(c3ccc(-c4ccc5ccccc5c4)cc3)c3ccc(-c4cc5ccc6ccc7ccccc7c6c5c5ccccc45)cc3)cc2)cc1]

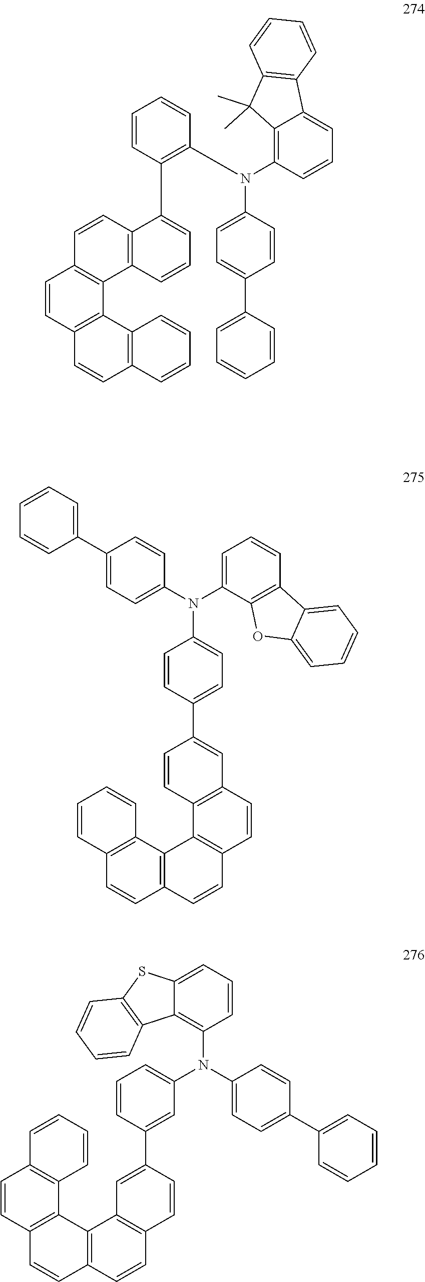 [SMILES: CC1(C)c2ccccc2-c2cccc(N(c3ccc(-c4ccccc4)cc3)c3ccccc3-c3cccc4c3ccc3ccc5ccc6ccccc6c5c34)c21 ; c1ccc(-c2ccc(N(c3ccc(-c4ccc5c(ccc6ccc7ccc8ccccc8c7c65)c4)cc3)c3cccc4c3oc3ccccc34)cc2)cc1 ; c1ccc(-c2ccc(N(c3cccc(-c4ccc5ccc6ccc7ccc8ccccc8c7c6c5c4)c3)c3cccc4sc5ccccc5c34)cc2)cc1]

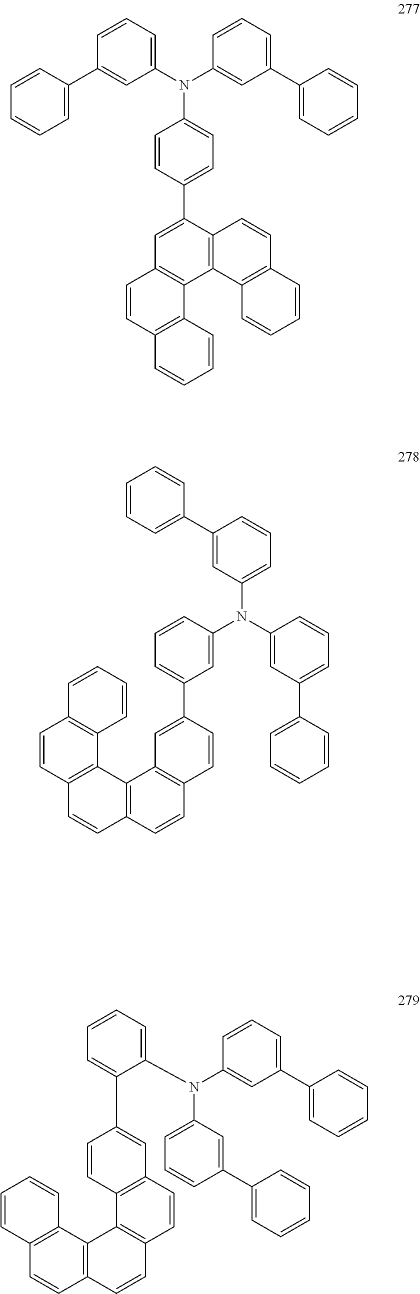 [SMILES: c1ccc(-c2cccc(N(c3ccc(-c4cc5ccc6ccccc6c5c5c4ccc4ccccc45)cc3)c3cccc(-c4ccccc4)c3)c2)cc1 ; c1ccc(-c2cccc(N(c3cccc(-c4ccccc4)c3)c3cccc(-c4ccc5ccc6ccc7ccc8ccccc8c7c6c5c4)c3)c2)cc1 ; c1ccc(-c2cccc(N(c3cccc(-c4ccccc4)c3)c3ccccc3-c3ccc4c(ccc5ccc6ccc7ccccc7c6c54)c3)c2)cc1]

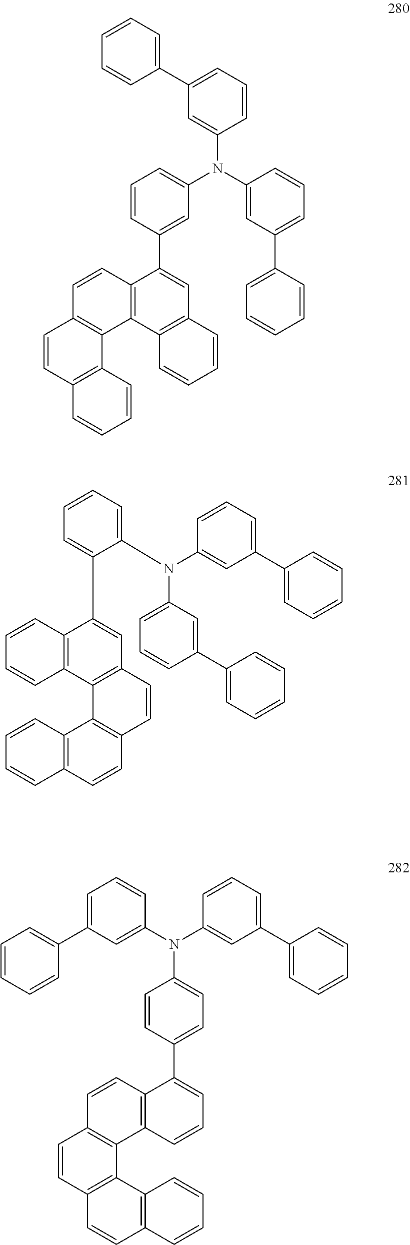 [SMILES: c1ccc(-c2cccc(N(c3cccc(-c4ccccc4)c3)c3cccc(-c4cc5ccccc5c5c4ccc4ccc6ccccc6c45)c3)c2)cc1 ; c1ccc(-c2cccc(N(c3cccc(-c4ccccc4)c3)c3ccccc3-c3cc4ccc5ccc6ccccc6c5c4c4ccccc34)c2)cc1 ; c1ccc(-c2cccc(N(c3ccc(-c4cccc5c4ccc4ccc6ccc7ccccc7c6c45)cc3)c3cccc(-c4ccccc4)c3)c2)cc1]

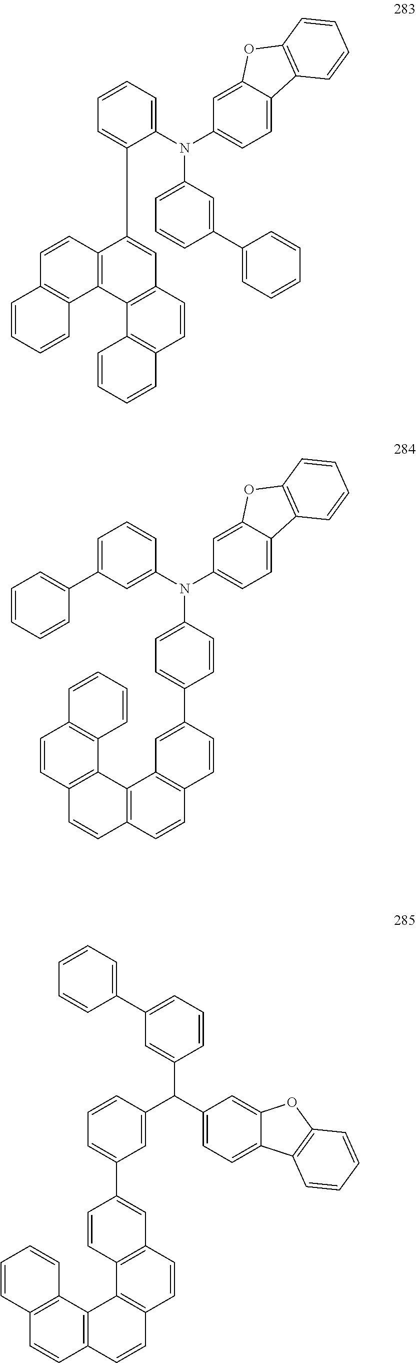 [SMILES: c1ccc(-c2cccc(N(c3ccc4c(c3)oc3ccccc34)c3ccccc3-c3cc4ccc5ccccc5c4c4c3ccc3ccccc34)c2)cc1 ; c1ccc(-c2cccc(N(c3ccc(-c4ccc5ccc6ccc7ccc8ccccc8c7c6c5c4)cc3)c3ccc4c(c3)oc3ccccc34)c2)cc1 ; c1ccc(-c2cccc(C(c3cccc(-c4ccc5c(ccc6ccc7ccc8ccccc8c7c65)c4)c3)c3ccc4c(c3)oc3ccccc34)c2)cc1]

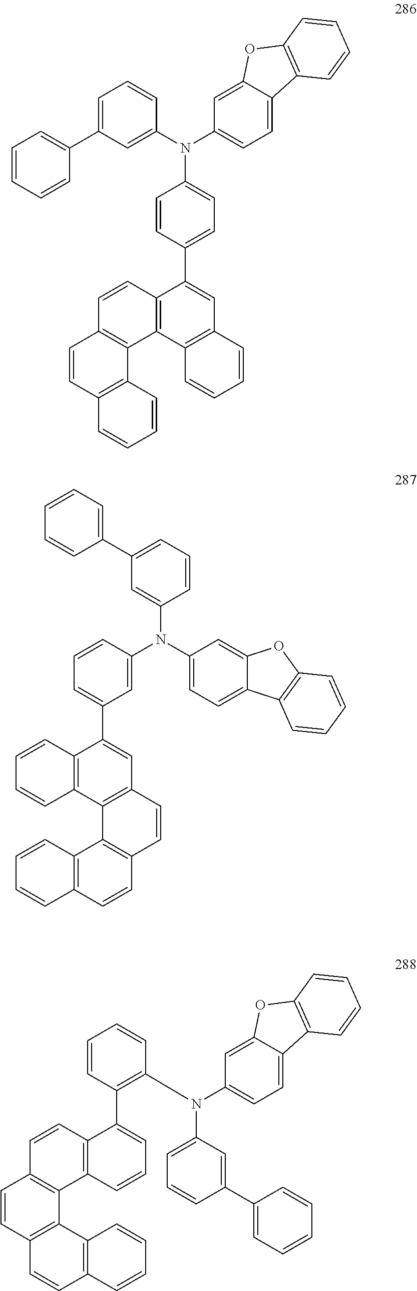 [SMILES: c1ccc(-c2cccc(N(c3ccc(-c4cc5ccccc5c5c4ccc4ccc6ccccc6c45)cc3)c3ccc4c(c3)oc3ccccc34)c2)cc1 ; c1ccc(-c2cccc(N(c3cccc(-c4cc5ccc6ccc7ccccc7c6c5c5ccccc45)c3)c3ccc4c(c3)oc3ccccc34)c2)cc1 ; c1ccc(-c2cccc(N(c3ccc4c(c3)oc3ccccc34)c3ccccc3-c3cccc4c3ccc3ccc5ccc6ccccc6c5c34)c2)cc1]

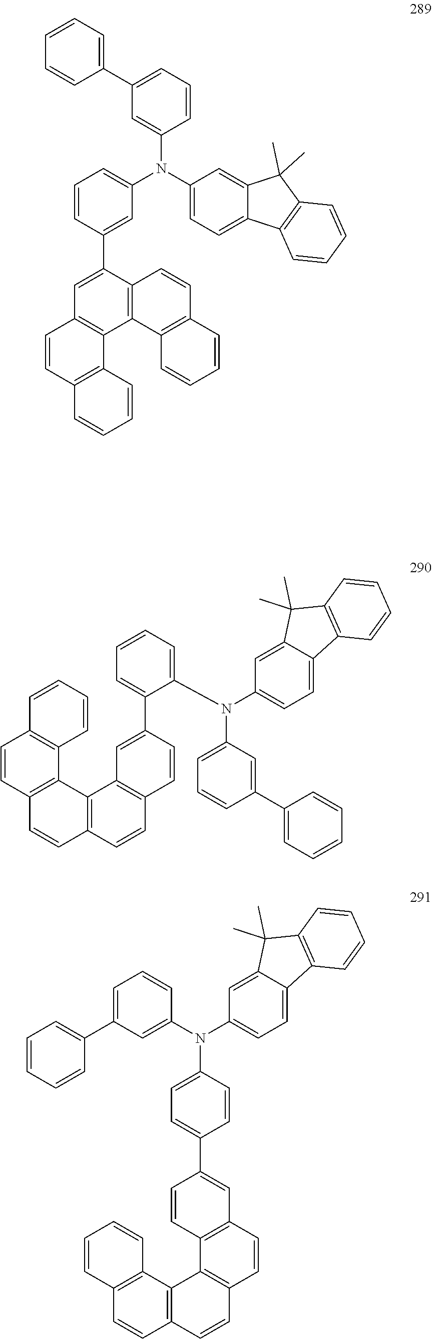 [SMILES: CC1(C)c2ccccc2-c2ccc(N(c3cccc(-c4ccccc4)c3)c3cccc(-c4cc5ccc6ccccc6c5c5c4ccc4ccccc45)c3)cc21 ; CC1(C)c2ccccc2-c2ccc(N(c3cccc(-c4ccccc4)c3)c3ccccc3-c3ccc4ccc5ccc6ccc7ccccc7c6c5c4c3)cc21 ; CC1(C)c2ccccc2-c2ccc(N(c3ccc(-c4ccc5c(ccc6ccc7ccc8ccccc8c7c65)c4)cc3)c3cccc(-c4ccccc4)c3)cc21]

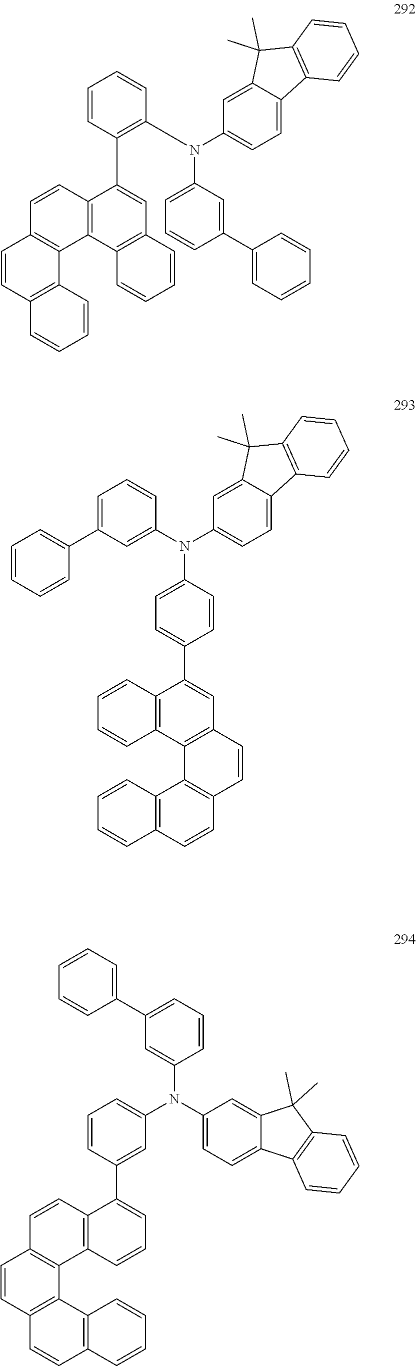 [SMILES: CC1(C)c2ccccc2-c2ccc(N(c3cccc(-c4ccccc4)c3)c3ccccc3-c3cc4ccccc4c4c3ccc3ccc5ccccc5c34)cc21 ; CC1(C)c2ccccc2-c2ccc(N(c3ccc(-c4cc5ccc6ccc7ccccc7c6c5c5ccccc45)cc3)c3cccc(-c4ccccc4)c3)cc21 ; CC1(C)c2ccccc2-c2ccc(N(c3cccc(-c4ccccc4)c3)c3cccc(-c4cccc5c4ccc4ccc6ccc7ccccc7c6c45)c3)cc21]

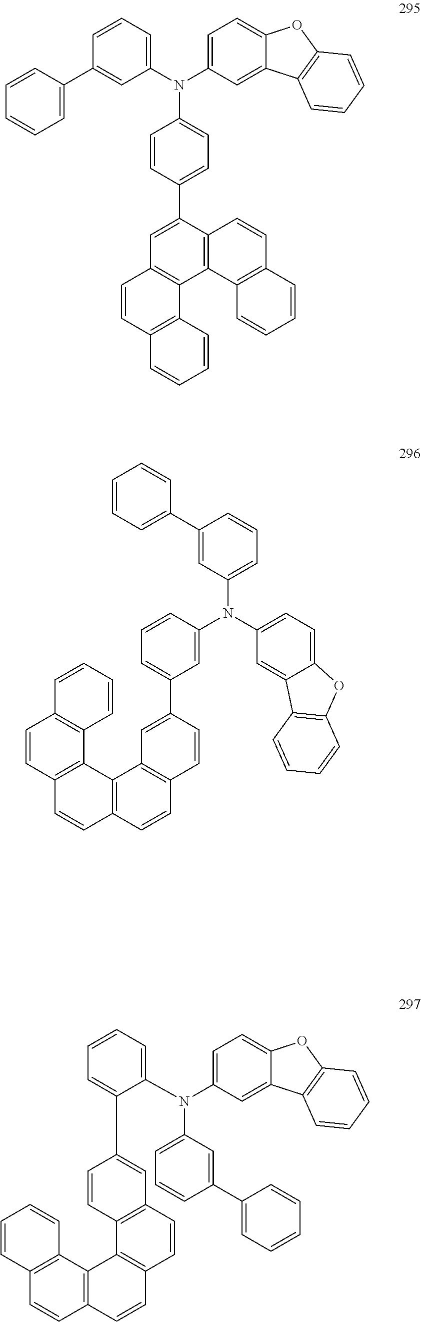 [SMILES: c1ccc(-c2cccc(N(c3ccc(-c4cc5ccc6ccccc6c5c5c4ccc4ccccc45)cc3)c3ccc4oc5ccccc5c4c3)c2)cc1 ; c1ccc(-c2cccc(N(c3cccc(-c4ccc5ccc6ccc7ccc8ccccc8c7c6c5c4)c3)c3ccc4oc5ccccc5c4c3)c2)cc1 ; c1ccc(-c2cccc(N(c3ccc4oc5ccccc5c4c3)c3ccccc3-c3ccc4c(ccc5ccc6ccc7ccccc7c6c54)c3)c2)cc1]

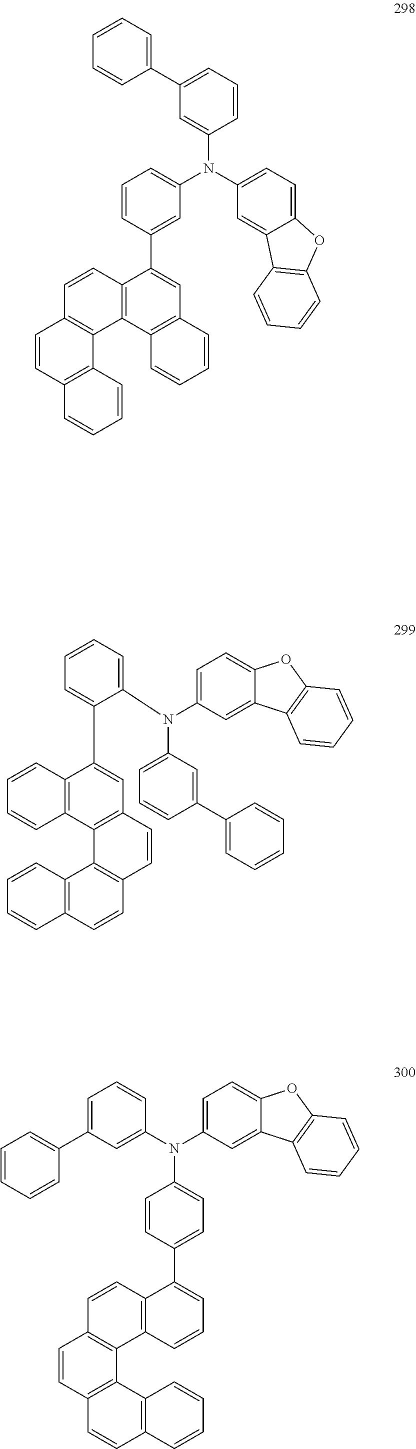 [SMILES: c1ccc(-c2cccc(N(c3cccc(-c4cc5ccccc5c5c4ccc4ccc6ccccc6c45)c3)c3ccc4oc5ccccc5c4c3)c2)cc1 ; c1ccc(-c2cccc(N(c3ccc4oc5ccccc5c4c3)c3ccccc3-c3cc4ccc5ccc6ccccc6c5c4c4ccccc34)c2)cc1 ; c1ccc(-c2cccc(N(c3ccc(-c4cccc5c4ccc4ccc6ccc7ccccc7c6c45)cc3)c3ccc4oc5ccccc5c4c3)c2)cc1]

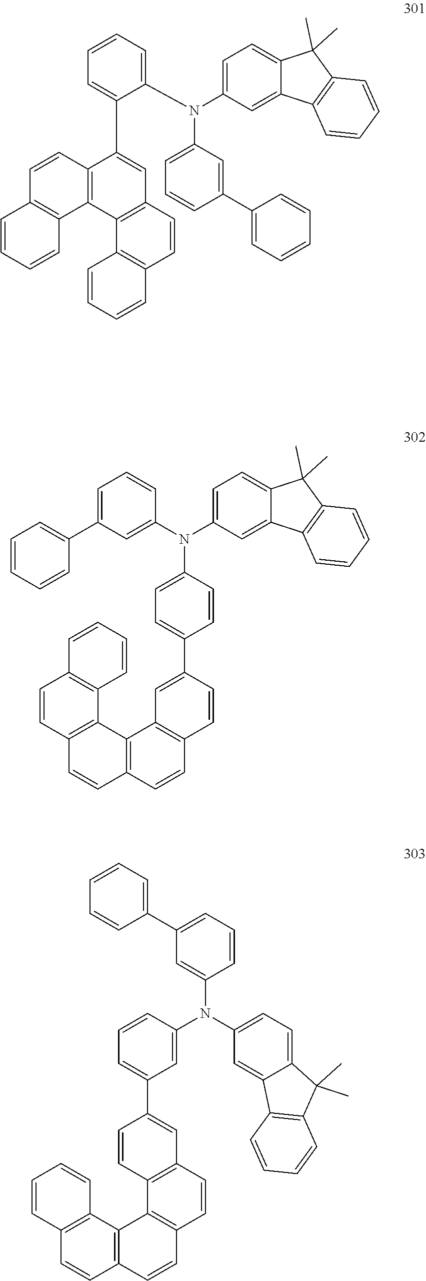 [SMILES: CC1(C)c2ccccc2-c2cc(N(c3cccc(-c4ccccc4)c3)c3ccccc3-c3cc4ccc5ccccc5c4c4c3ccc3ccccc34)ccc21 ; CC1(C)c2ccccc2-c2cc(N(c3ccc(-c4ccc5ccc6ccc7ccc8ccccc8c7c6c5c4)cc3)c3cccc(-c4ccccc4)c3)ccc21 ; CC1(C)c2ccccc2-c2cc(N(c3cccc(-c4ccccc4)c3)c3cccc(-c4ccc5c(ccc6ccc7ccc8ccccc8c7c65)c4)c3)ccc21]

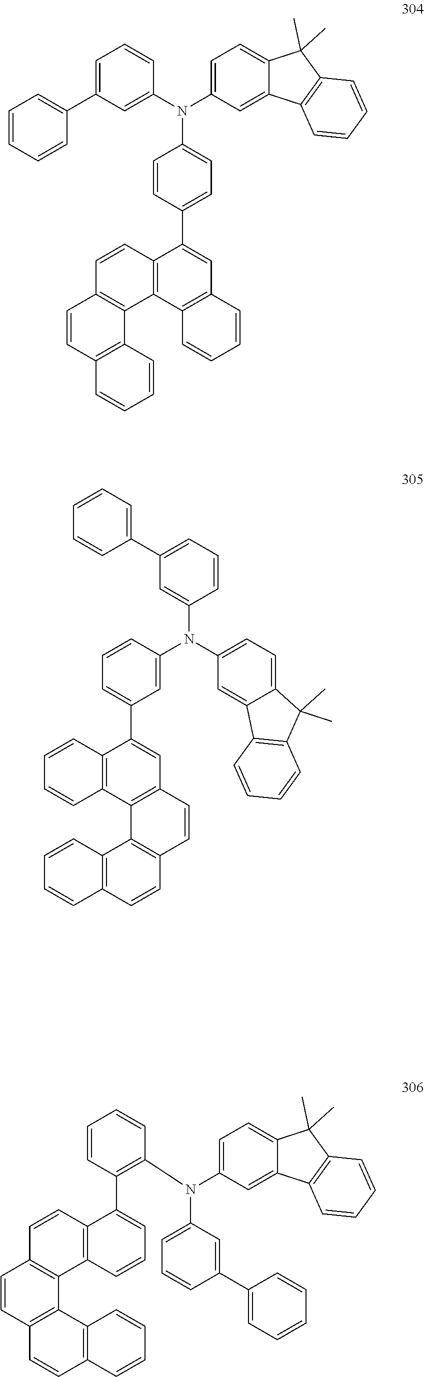 [SMILES: CC1(C)c2ccccc2-c2cc(N(c3ccc(-c4cc5ccccc5c5c4ccc4ccc6ccccc6c45)cc3)c3cccc(-c4ccccc4)c3)ccc21 ; CC1(C)c2ccccc2-c2cc(N(c3cccc(-c4ccccc4)c3)c3cccc(-c4cc5ccc6ccc7ccccc7c6c5c5ccccc45)c3)ccc21 ; CC1(C)c2ccccc2-c2cc(N(c3cccc(-c4ccccc4)c3)c3ccccc3-c3cccc4c3ccc3ccc5ccc6ccccc6c5c34)ccc21]

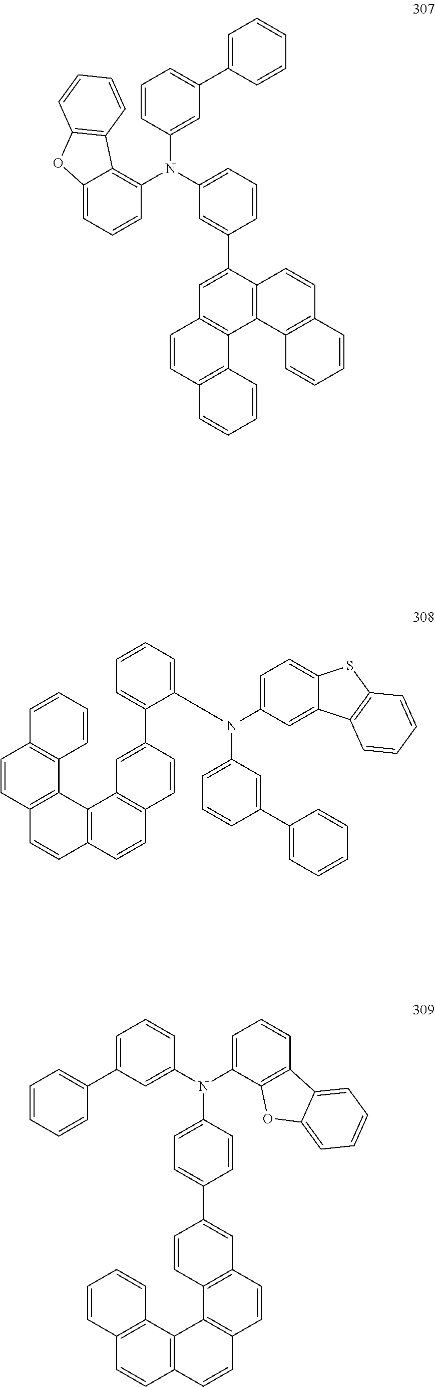 [SMILES: c1ccc(-c2cccc(N(c3cccc(-c4cc5ccc6ccccc6c5c5c4ccc4ccccc45)c3)c3cccc4oc5ccccc5c34)c2)cc1 ; c1ccc(-c2cccc(N(c3ccc4sc5ccccc5c4c3)c3ccccc3-c3ccc4ccc5ccc6ccc7ccccc7c6c5c4c3)c2)cc1 ; c1ccc(-c2cccc(N(c3ccc(-c4ccc5c(ccc6ccc7ccc8ccccc8c7c65)c4)cc3)c3cccc4c3oc3ccccc34)c2)cc1]

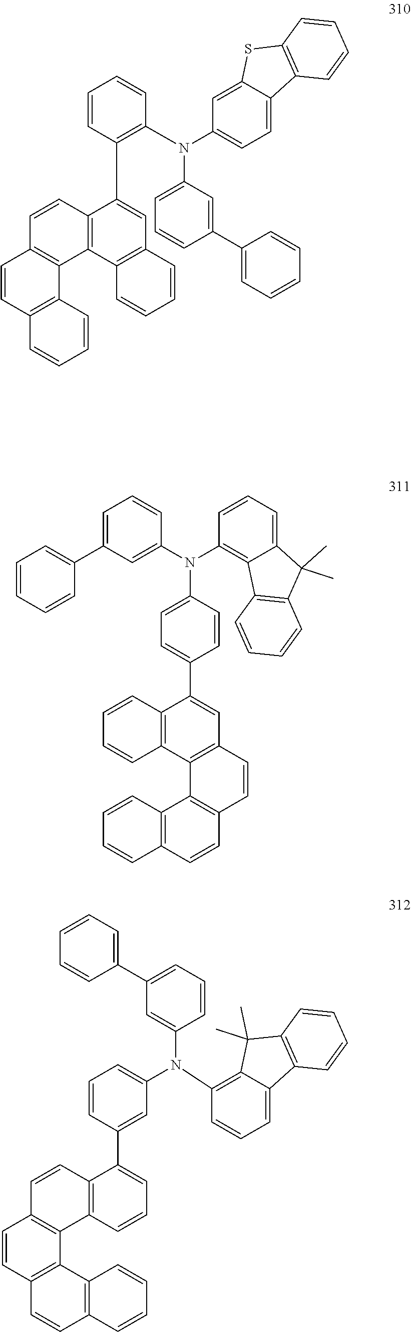 [SMILES: c1ccc(-c2cccc(N(c3ccc4c(c3)sc3ccccc34)c3ccccc3-c3cc4ccccc4c4c3ccc3ccc5ccccc5c34)c2)cc1 ; CC1(C)c2ccccc2-c2c(N(c3ccc(-c4cc5ccc6ccc7ccccc7c6c5c5ccccc45)cc3)c3cccc(-c4ccccc4)c3)cccc21 ; CC1(C)c2ccccc2-c2cccc(N(c3cccc(-c4ccccc4)c3)c3cccc(-c4cccc5c4ccc4ccc6ccc7ccccc7c6c45)c3)c21]

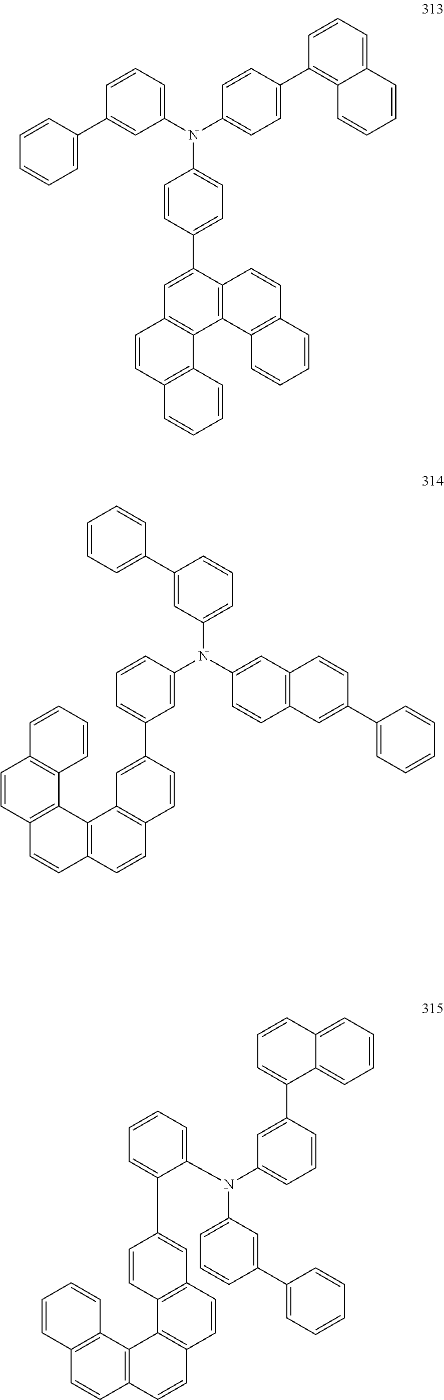 [SMILES: c1ccc(-c2cccc(N(c3ccc(-c4cccc5ccccc45)cc3)c3ccc(-c4cc5ccc6ccccc6c5c5c4ccc4ccccc45)cc3)c2)cc1 ; c1ccc(-c2cccc(N(c3cccc(-c4ccc5ccc6ccc7ccc8ccccc8c7c6c5c4)c3)c3ccc4cc(-c5ccccc5)ccc4c3)c2)cc1 ; c1ccc(-c2cccc(N(c3cccc(-c4cccc5ccccc45)c3)c3ccccc3-c3ccc4c(ccc5ccc6ccc7ccccc7c6c54)c3)c2)cc1]

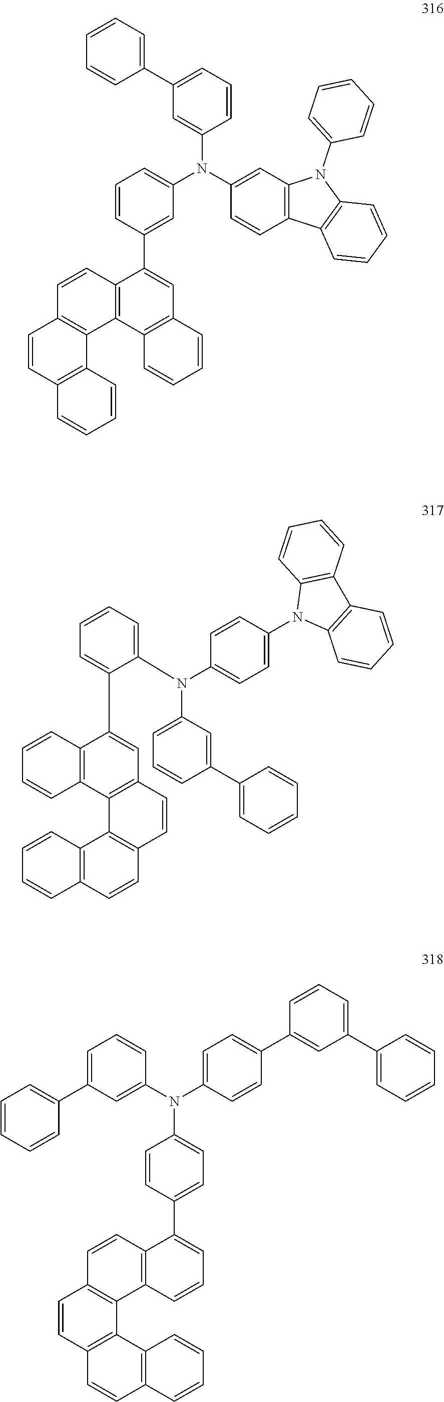 [SMILES: c1ccc(-c2cccc(N(c3cccc(-c4cc5ccccc5c5c4ccc4ccc6ccccc6c45)c3)c3ccc4c5ccccc5n(-c5ccccc5)c4c3)c2)cc1 ; c1ccc(-c2cccc(N(c3ccc(-n4c5ccccc5c5ccccc54)cc3)c3ccccc3-c3cc4ccc5ccc6ccccc6c5c4c4ccccc34)c2)cc1 ; c1ccc(-c2cccc(-c3ccc(N(c4ccc(-c5cccc6c5ccc5ccc7ccc8ccccc8c7c56)cc4)c4cccc(-c5ccccc5)c4)cc3)c2)cc1]

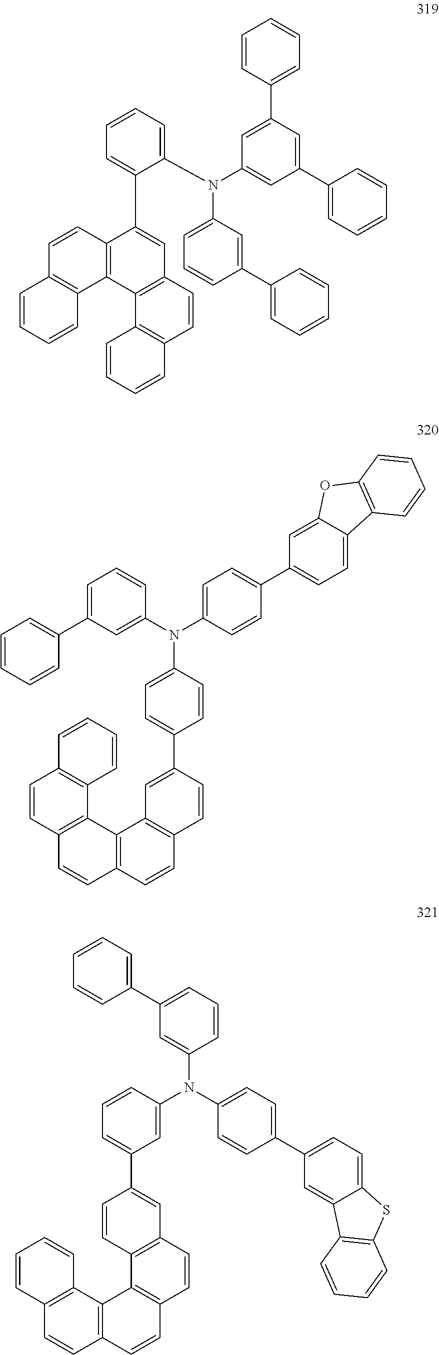 [SMILES: c1ccc(-c2cccc(N(c3cc(-c4ccccc4)cc(-c4ccccc4)c3)c3ccccc3-c3cc4ccc5ccccc5c4c4c3ccc3ccccc34)c2)cc1 ; c1ccc(-c2cccc(N(c3ccc(-c4ccc5c(c4)oc4ccccc45)cc3)c3ccc(-c4ccc5ccc6ccc7ccc8ccccc8c7c6c5c4)cc3)c2)cc1 ; c1ccc(-c2cccc(N(c3ccc(-c4ccc5sc6ccccc6c5c4)cc3)c3cccc(-c4ccc5c(ccc6ccc7ccc8ccccc8c7c65)c4)c3)c2)cc1]

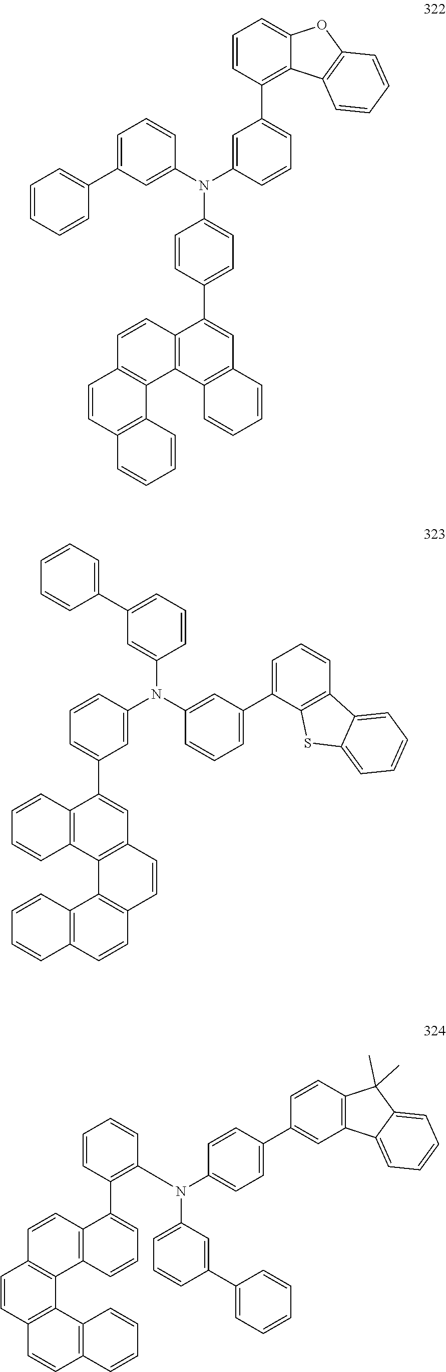 [SMILES: c1ccc(-c2cccc(N(c3ccc(-c4cc5ccccc5c5c4ccc4ccc6ccccc6c45)cc3)c3cccc(-c4cccc5oc6ccccc6c45)c3)c2)cc1 ; c1ccc(-c2cccc(N(c3cccc(-c4cc5ccc6ccc7ccccc7c6c5c5ccccc45)c3)c3cccc(-c4cccc5c4sc4ccccc45)c3)c2)cc1 ; CC1(C)c2ccccc2-c2cc(-c3ccc(N(c4cccc(-c5ccccc5)c4)c4ccccc4-c4cccc5c4ccc4ccc6ccc7ccccc7c6c45)cc3)ccc21]

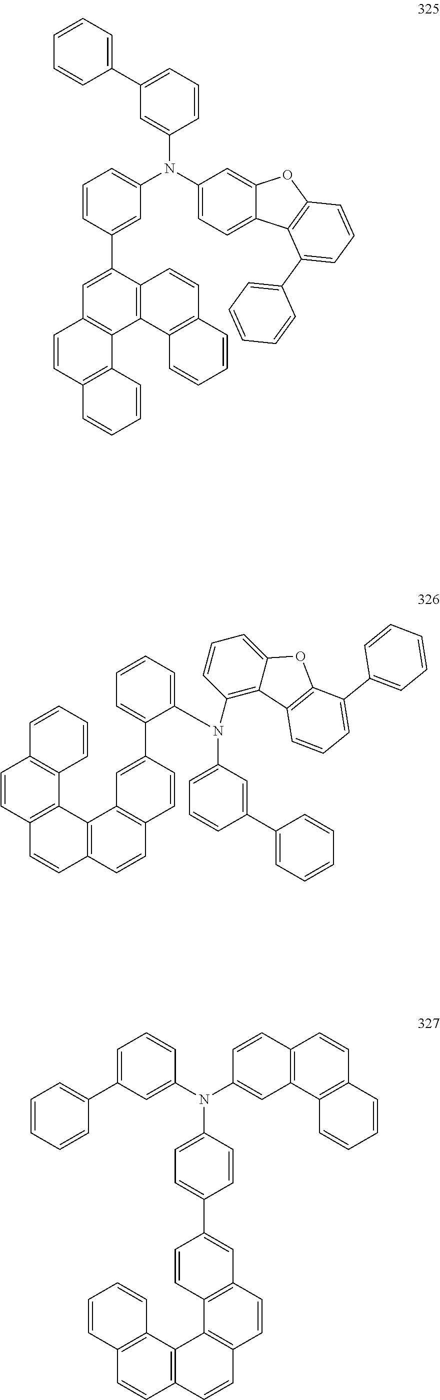 [SMILES: c1ccc(-c2cccc(N(c3cccc(-c4cc5ccc6ccccc6c5c5c4ccc4ccccc45)c3)c3ccc4c(c3)oc3cccc(-c5ccccc5)c34)c2)cc1 ; c1ccc(-c2cccc(N(c3ccccc3-c3ccc4ccc5ccc6ccc7ccccc7c6c5c4c3)c3cccc4oc5c(-c6ccccc6)cccc5c34)c2)cc1 ; c1ccc(-c2cccc(N(c3ccc(-c4ccc5c(ccc6ccc7ccc8ccccc8c7c65)c4)cc3)c3ccc4ccc5ccccc5c4c3)c2)cc1]

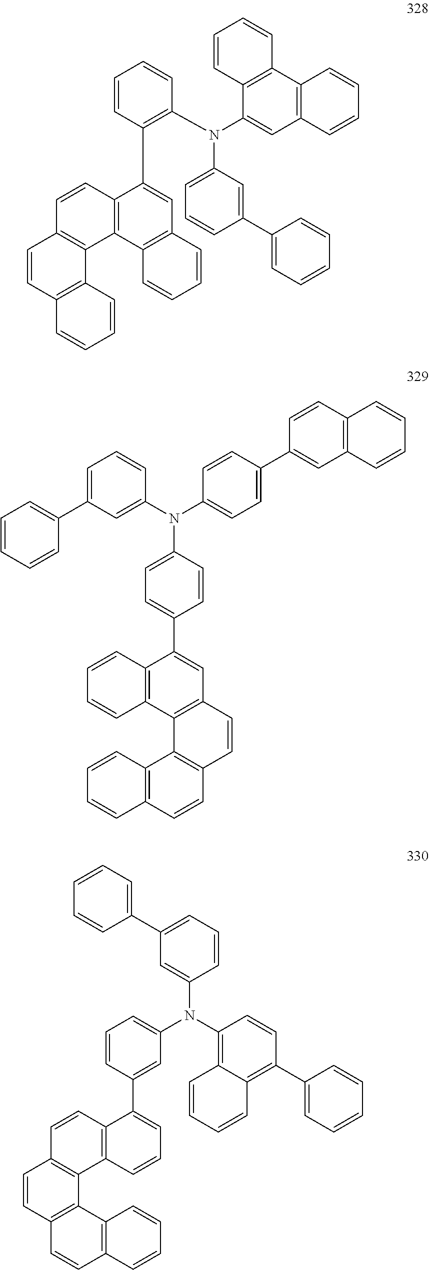 [SMILES: c1ccc(-c2cccc(N(c3ccccc3-c3cc4ccccc4c4c3ccc3ccc5ccccc5c34)c3cc4ccccc4c4ccccc34)c2)cc1 ; c1ccc(-c2cccc(N(c3ccc(-c4ccc5ccccc5c4)cc3)c3ccc(-c4cc5ccc6ccc7ccccc7c6c5c5ccccc45)cc3)c2)cc1 ; c1ccc(-c2cccc(N(c3cccc(-c4cccc5c4ccc4ccc6ccc7ccccc7c6c45)c3)c3ccc(-c4ccccc4)c4ccccc34)c2)cc1]

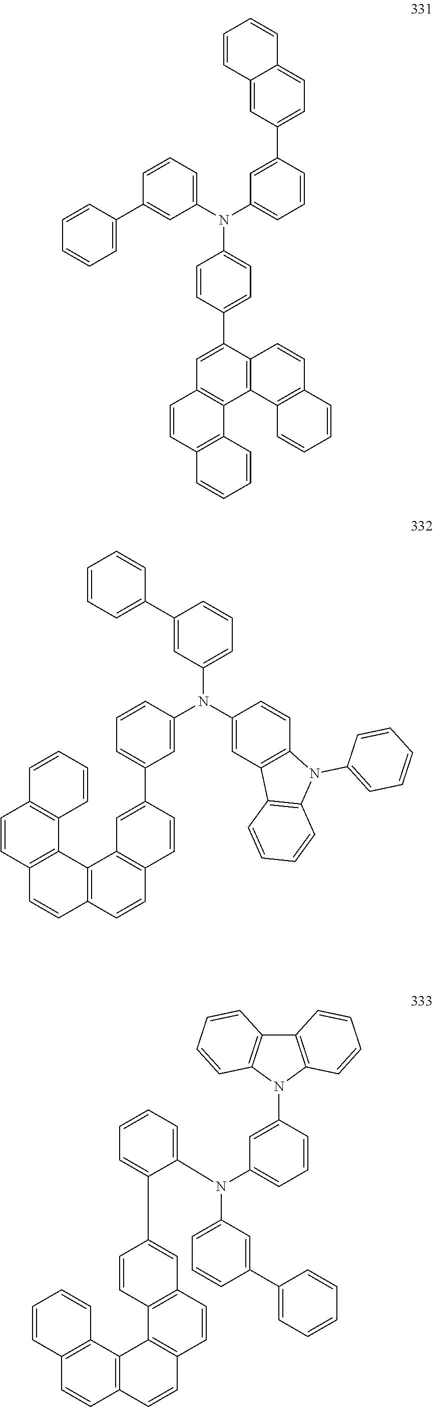 [SMILES: c1ccc(-c2cccc(N(c3ccc(-c4cc5ccc6ccccc6c5c5c4ccc4ccccc45)cc3)c3cccc(-c4ccc5ccccc5c4)c3)c2)cc1 ; c1ccc(-c2cccc(N(c3cccc(-c4ccc5ccc6ccc7ccc8ccccc8c7c6c5c4)c3)c3ccc4c(c3)c3ccccc3n4-c3ccccc3)c2)cc1 ; c1ccc(-c2cccc(N(c3cccc(-n4c5ccccc5c5ccccc54)c3)c3ccccc3-c3ccc4c(ccc5ccc6ccc7ccccc7c6c54)c3)c2)cc1]

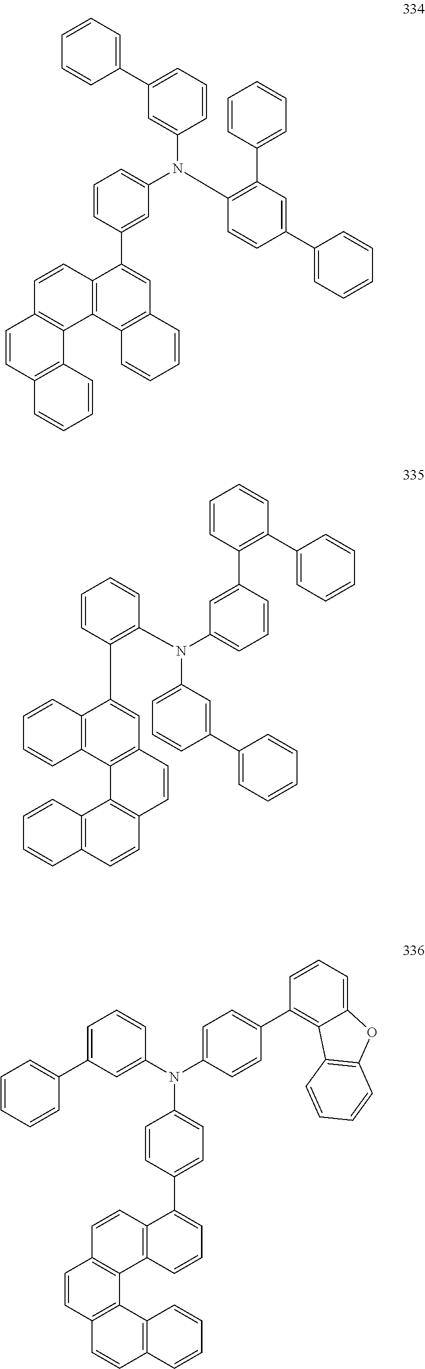 [SMILES: c1ccc(-c2cccc(N(c3cccc(-c4cc5ccccc5c5c4ccc4ccc6ccccc6c45)c3)c3ccc(-c4ccccc4)cc3-c3ccccc3)c2)cc1 ; c1ccc(-c2cccc(N(c3cccc(-c4ccccc4-c4ccccc4)c3)c3ccccc3-c3cc4ccc5ccc6ccccc6c5c4c4ccccc34)c2)cc1 ; c1ccc(-c2cccc(N(c3ccc(-c4cccc5c4ccc4ccc6ccc7ccccc7c6c45)cc3)c3ccc(-c4cccc5oc6ccccc6c45)cc3)c2)cc1]

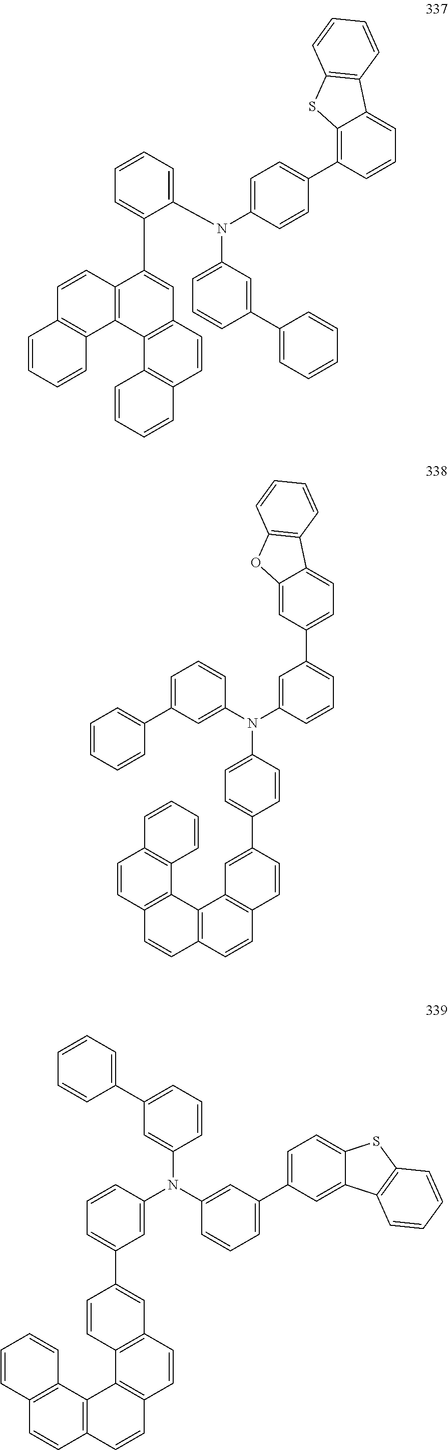 [SMILES: c1ccc(-c2cccc(N(c3ccc(-c4cccc5c4sc4ccccc45)cc3)c3ccccc3-c3cc4ccc5ccccc5c4c4c3ccc3ccccc34)c2)cc1 ; c1ccc(-c2cccc(N(c3ccc(-c4ccc5ccc6ccc7ccc8ccccc8c7c6c5c4)cc3)c3cccc(-c4ccc5c(c4)oc4ccccc45)c3)c2)cc1 ; c1ccc(-c2cccc(N(c3cccc(-c4ccc5c(ccc6ccc7ccc8ccccc8c7c65)c4)c3)c3cccc(-c4ccc5sc6ccccc6c5c4)c3)c2)cc1]

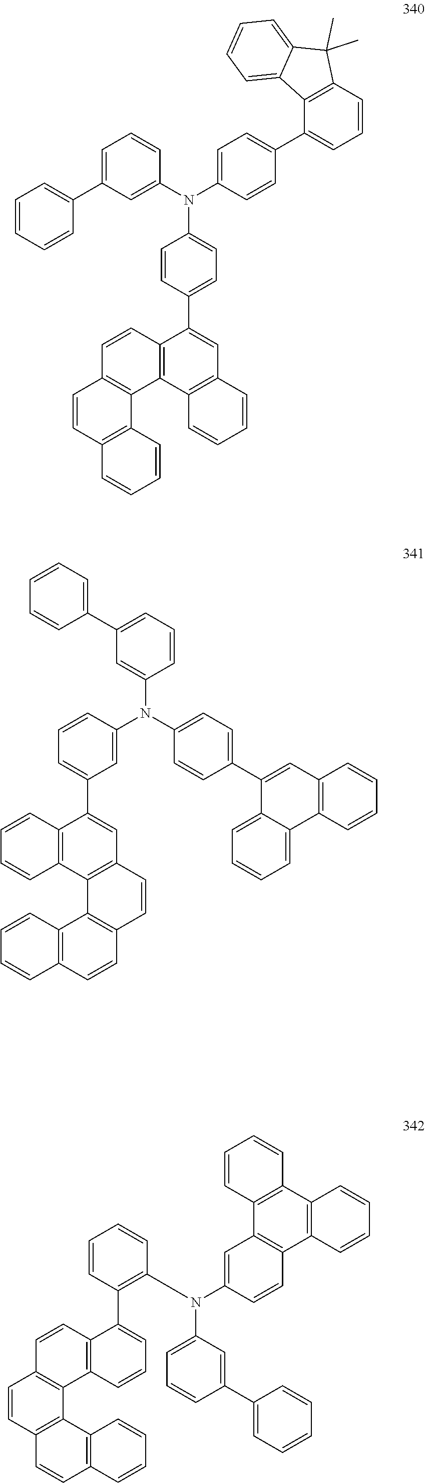 [SMILES: CC1(C)c2ccccc2-c2c(-c3ccc(N(c4ccc(-c5cc6ccccc6c6c5ccc5ccc7ccccc7c56)cc4)c4cccc(-c5ccccc5)c4)cc3)cccc21 ; c1ccc(-c2cccc(N(c3ccc(-c4cc5ccccc5c5ccccc45)cc3)c3cccc(-c4cc5ccc6ccc7ccccc7c6c5c5ccccc45)c3)c2)cc1 ; c1ccc(-c2cccc(N(c3ccc4c5ccccc5c5ccccc5c4c3)c3ccccc3-c3cccc4c3ccc3ccc5ccc6ccccc6c5c34)c2)cc1]

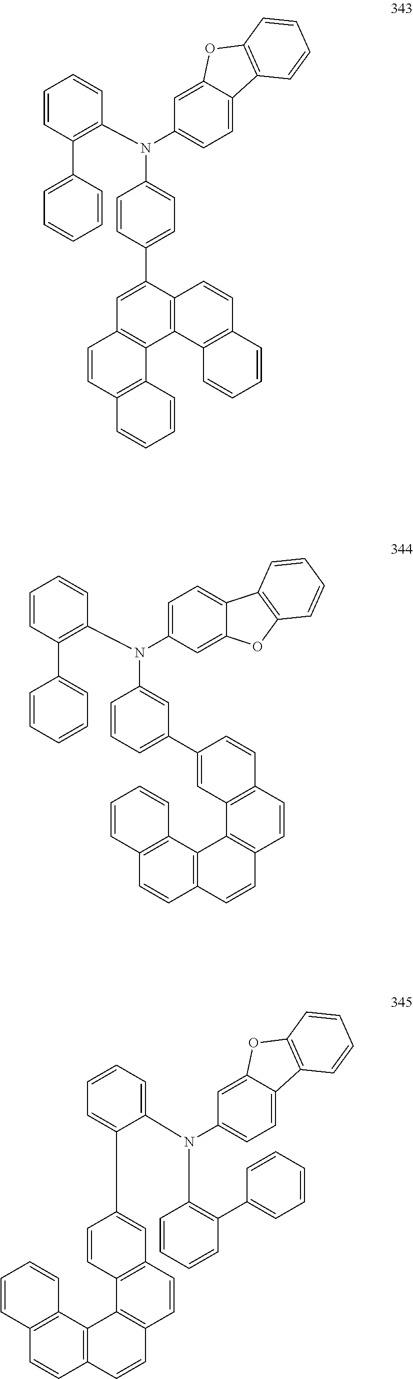 [SMILES: c1ccc(-c2ccccc2N(c2ccc(-c3cc4ccc5ccccc5c4c4c3ccc3ccccc34)cc2)c2ccc3c(c2)oc2ccccc23)cc1 ; c1ccc(-c2ccccc2N(c2cccc(-c3ccc4ccc5ccc6ccc7ccccc7c6c5c4c3)c2)c2ccc3c(c2)oc2ccccc23)cc1 ; c1ccc(-c2ccccc2N(c2ccc3c(c2)oc2ccccc23)c2ccccc2-c2ccc3c(ccc4ccc5ccc6ccccc6c5c43)c2)cc1]

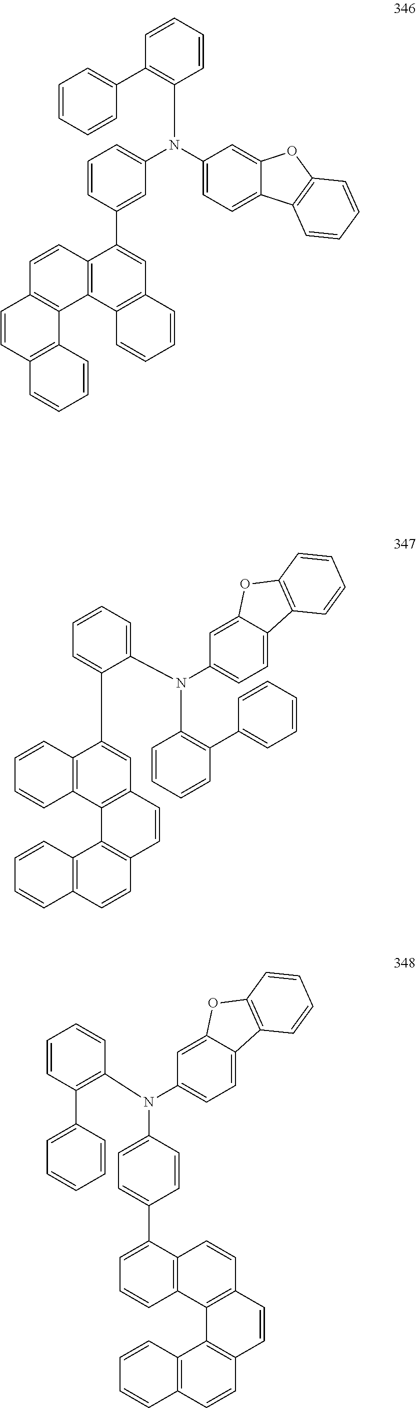 [SMILES: c1ccc(-c2ccccc2N(c2cccc(-c3cc4ccccc4c4c3ccc3ccc5ccccc5c34)c2)c2ccc3c(c2)oc2ccccc23)cc1 ; c1ccc(-c2ccccc2N(c2ccc3c(c2)oc2ccccc23)c2ccccc2-c2cc3ccc4ccc5ccccc5c4c3c3ccccc23)cc1 ; c1ccc(-c2ccccc2N(c2ccc(-c3cccc4c3ccc3ccc5ccc6ccccc6c5c34)cc2)c2ccc3c(c2)oc2ccccc23)cc1]

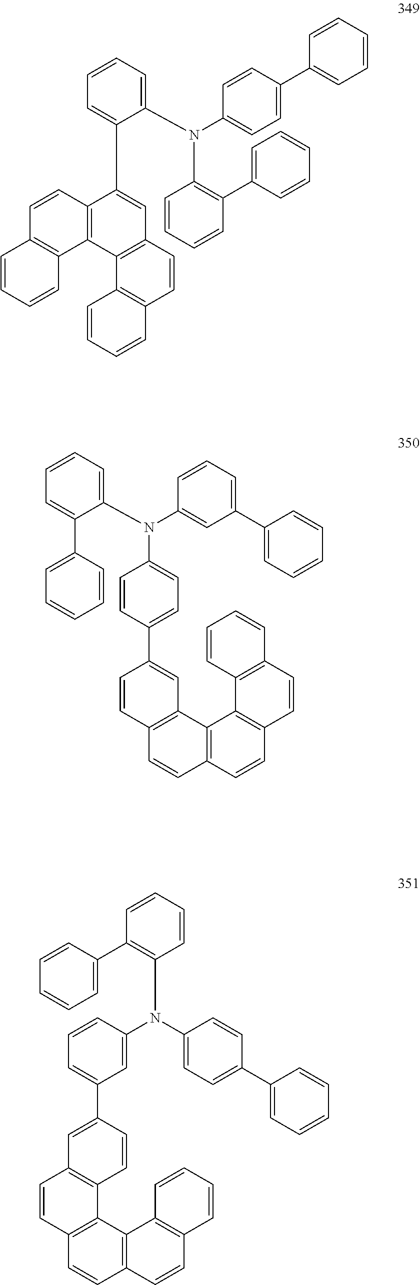 [SMILES: c1ccc(-c2ccc(N(c3ccccc3-c3ccccc3)c3ccccc3-c3cc4ccc5ccccc5c4c4c3ccc3ccccc34)cc2)cc1 ; c1ccc(-c2cccc(N(c3ccc(-c4ccc5ccc6ccc7ccc8ccccc8c7c6c5c4)cc3)c3ccccc3-c3ccccc3)c2)cc1 ; c1ccc(-c2ccc(N(c3cccc(-c4ccc5c(ccc6ccc7ccc8ccccc8c7c65)c4)c3)c3ccccc3-c3ccccc3)cc2)cc1]

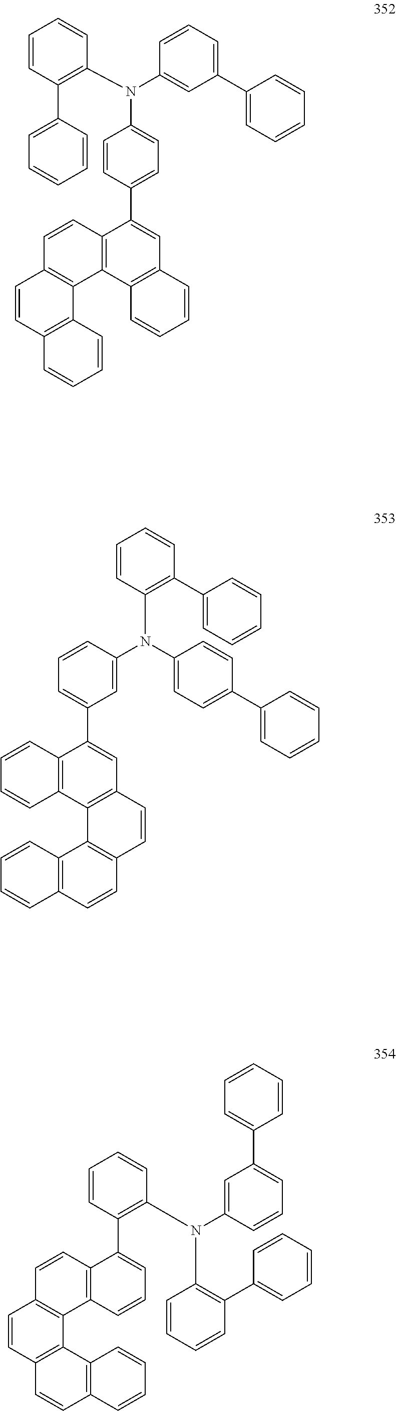 [SMILES: c1ccc(-c2cccc(N(c3ccc(-c4cc5ccccc5c5c4ccc4ccc6ccccc6c45)cc3)c3ccccc3-c3ccccc3)c2)cc1 ; c1ccc(-c2ccc(N(c3cccc(-c4cc5ccc6ccc7ccccc7c6c5c5ccccc45)c3)c3ccccc3-c3ccccc3)cc2)cc1 ; c1ccc(-c2cccc(N(c3ccccc3-c3ccccc3)c3ccccc3-c3cccc4c3ccc3ccc5ccc6ccccc6c5c34)c2)cc1]

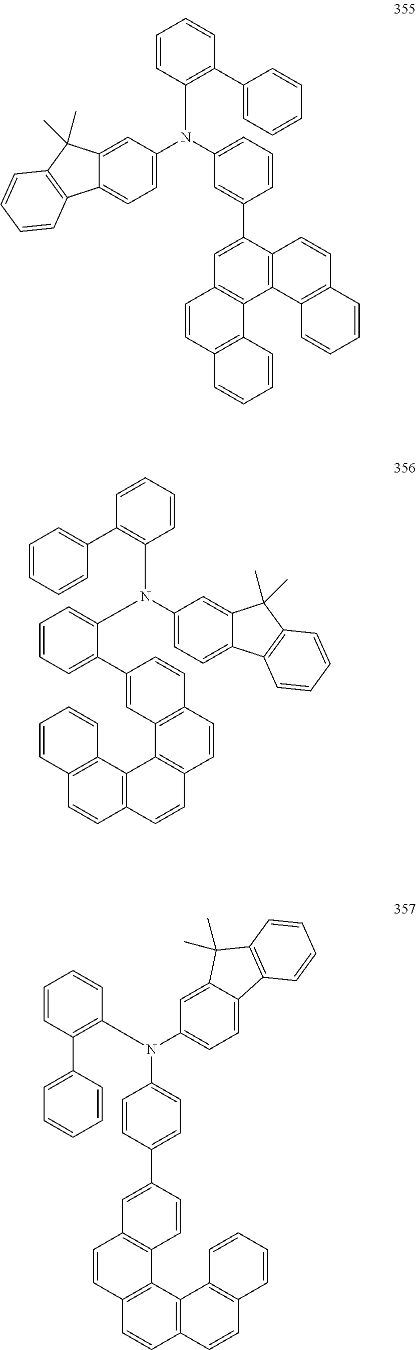 [SMILES: CC1(C)c2ccccc2-c2ccc(N(c3cccc(-c4cc5ccc6ccccc6c5c5c4ccc4ccccc45)c3)c3ccccc3-c3ccccc3)cc21 ; CC1(C)c2ccccc2-c2ccc(N(c3ccccc3-c3ccccc3)c3ccccc3-c3ccc4ccc5ccc6ccc7ccccc7c6c5c4c3)cc21 ; CC1(C)c2ccccc2-c2ccc(N(c3ccc(-c4ccc5c(ccc6ccc7ccc8ccccc8c7c65)c4)cc3)c3ccccc3-c3ccccc3)cc21]

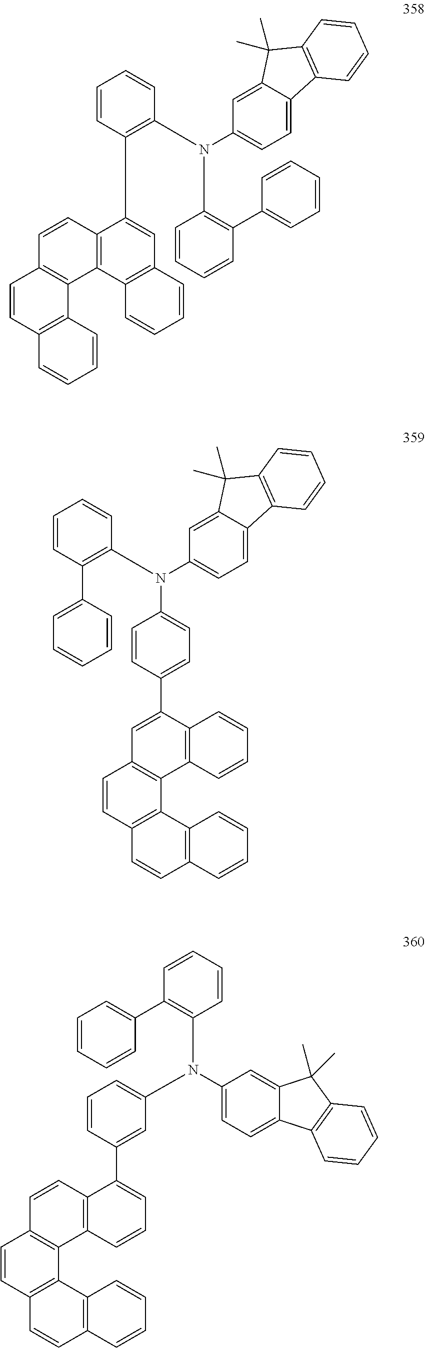 [SMILES: CC1(C)c2ccccc2-c2ccc(N(c3ccccc3-c3ccccc3)c3ccccc3-c3cc4ccccc4c4c3ccc3ccc5ccccc5c34)cc21 ; CC1(C)c2ccccc2-c2ccc(N(c3ccc(-c4cc5ccc6ccc7ccccc7c6c5c5ccccc45)cc3)c3ccccc3-c3ccccc3)cc21 ; CC1(C)c2ccccc2-c2ccc(N(c3cccc(-c4cccc5c4ccc4ccc6ccc7ccccc7c6c45)c3)c3ccccc3-c3ccccc3)cc21]

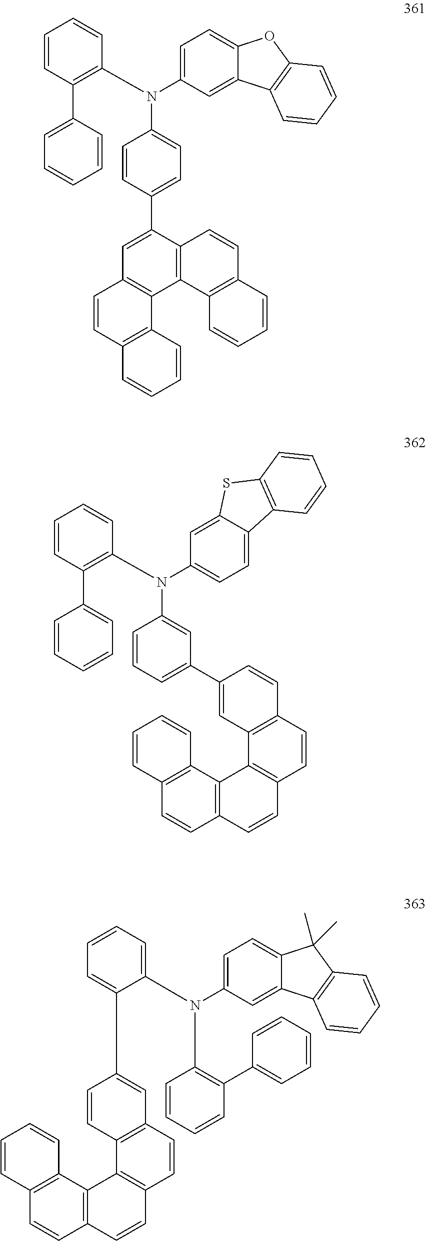 [SMILES: c1ccc(-c2ccccc2N(c2ccc(-c3cc4ccc5ccccc5c4c4c3ccc3ccccc34)cc2)c2ccc3oc4ccccc4c3c2)cc1 ; c1ccc(-c2ccccc2N(c2cccc(-c3ccc4ccc5ccc6ccc7ccccc7c6c5c4c3)c2)c2ccc3c(c2)sc2ccccc23)cc1 ; CC1(C)c2ccccc2-c2cc(N(c3ccccc3-c3ccccc3)c3ccccc3-c3ccc4c(ccc5ccc6ccc7ccccc7c6c54)c3)ccc21]

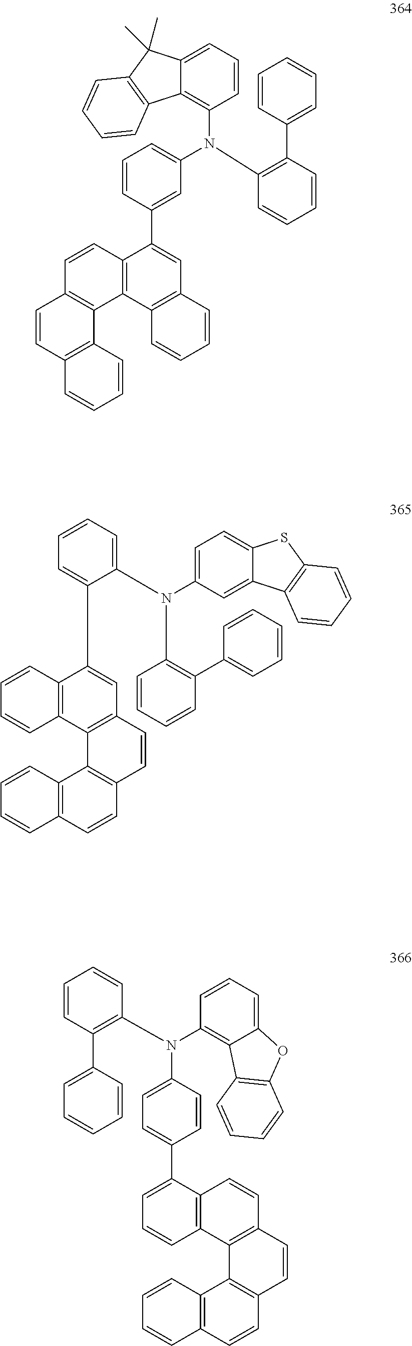 [SMILES: CC1(C)c2ccccc2-c2c(N(c3cccc(-c4cc5ccccc5c5c4ccc4ccc6ccccc6c45)c3)c3ccccc3-c3ccccc3)cccc21 ; c1ccc(-c2ccccc2N(c2ccc3sc4ccccc4c3c2)c2ccccc2-c2cc3ccc4ccc5ccccc5c4c3c3ccccc23)cc1 ; c1ccc(-c2ccccc2N(c2ccc(-c3cccc4c3ccc3ccc5ccc6ccccc6c5c34)cc2)c2cccc3oc4ccccc4c23)cc1]

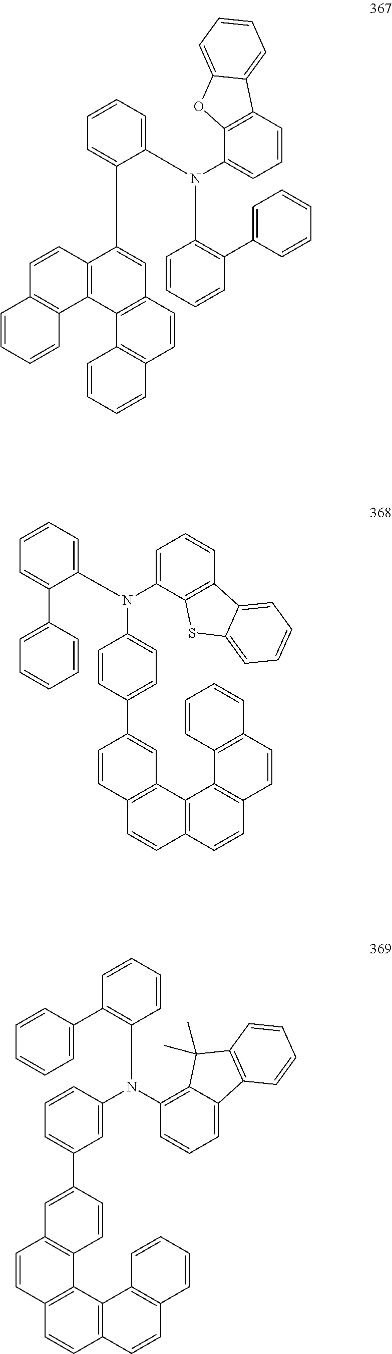 [SMILES: c1ccc(-c2ccccc2N(c2ccccc2-c2cc3ccc4ccccc4c3c3c2ccc2ccccc23)c2cccc3c2oc2ccccc23)cc1 ; c1ccc(-c2ccccc2N(c2ccc(-c3ccc4ccc5ccc6ccc7ccccc7c6c5c4c3)cc2)c2cccc3c2sc2ccccc23)cc1 ; CC1(C)c2ccccc2-c2cccc(N(c3cccc(-c4ccc5c(ccc6ccc7ccc8ccccc8c7c65)c4)c3)c3ccccc3-c3ccccc3)c21]

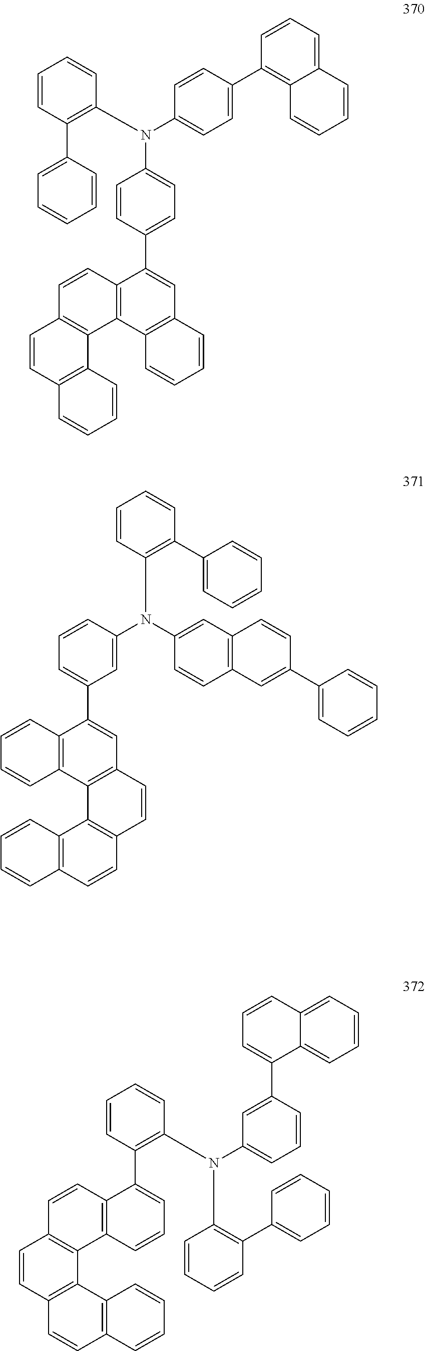 [SMILES: c1ccc(-c2ccccc2N(c2ccc(-c3cccc4ccccc34)cc2)c2ccc(-c3cc4ccccc4c4c3ccc3ccc5ccccc5c34)cc2)cc1 ; c1ccc(-c2ccc3cc(N(c4cccc(-c5cc6ccc7ccc8ccccc8c7c6c6ccccc56)c4)c4ccccc4-c4ccccc4)ccc3c2)cc1 ; c1ccc(-c2ccccc2N(c2cccc(-c3cccc4ccccc34)c2)c2ccccc2-c2cccc3c2ccc2ccc4ccc5ccccc5c4c23)cc1]

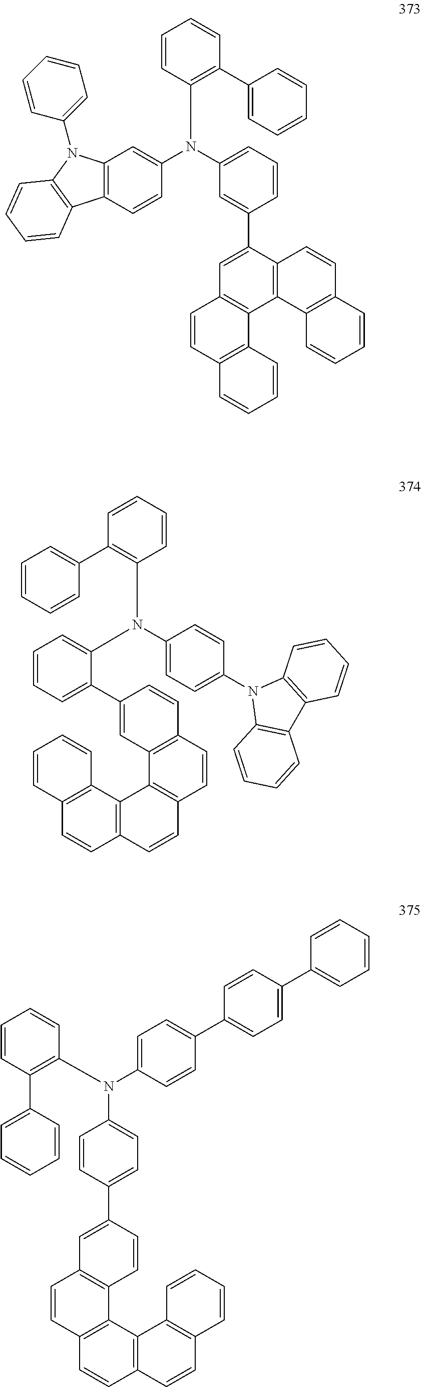 [SMILES: c1ccc(-c2ccccc2N(c2cccc(-c3cc4ccc5ccccc5c4c4c3ccc3ccccc34)c2)c2ccc3c4ccccc4n(-c4ccccc4)c3c2)cc1 ; c1ccc(-c2ccccc2N(c2ccc(-n3c4ccccc4c4ccccc43)cc2)c2ccccc2-c2ccc3ccc4ccc5ccc6ccccc6c5c4c3c2)cc1 ; c1ccc(-c2ccc(-c3ccc(N(c4ccc(-c5ccc6c(ccc7ccc8ccc9ccccc9c8c76)c5)cc4)c4ccccc4-c4ccccc4)cc3)cc2)cc1]

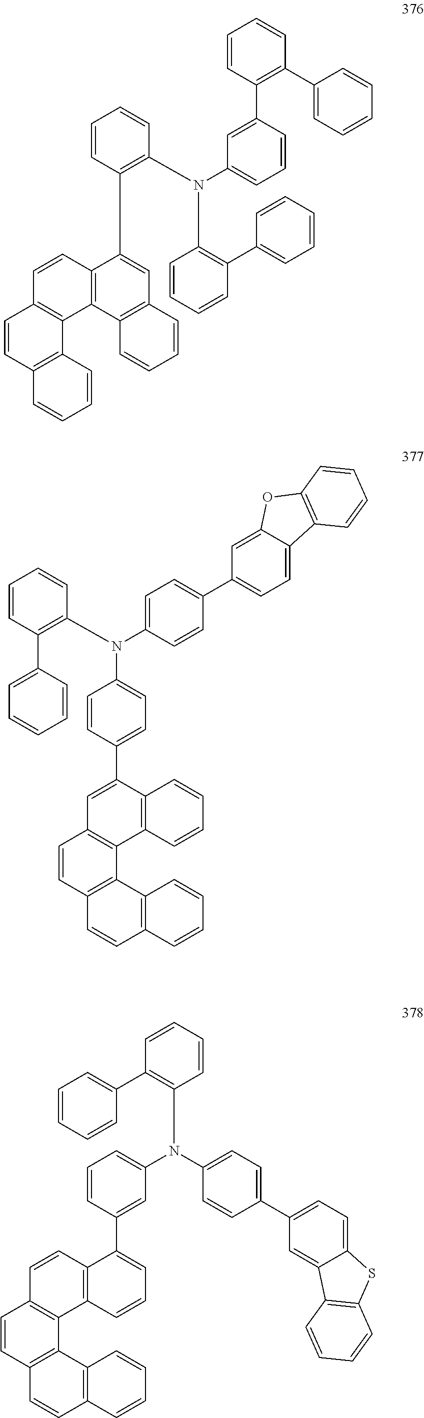 [SMILES: c1ccc(-c2ccccc2-c2cccc(N(c3ccccc3-c3ccccc3)c3ccccc3-c3cc4ccccc4c4c3ccc3ccc5ccccc5c34)c2)cc1 ; c1ccc(-c2ccccc2N(c2ccc(-c3ccc4c(c3)oc3ccccc34)cc2)c2ccc(-c3cc4ccc5ccc6ccccc6c5c4c4ccccc34)cc2)cc1 ; c1ccc(-c2ccccc2N(c2ccc(-c3ccc4sc5ccccc5c4c3)cc2)c2cccc(-c3cccc4c3ccc3ccc5ccc6ccccc6c5c34)c2)cc1]

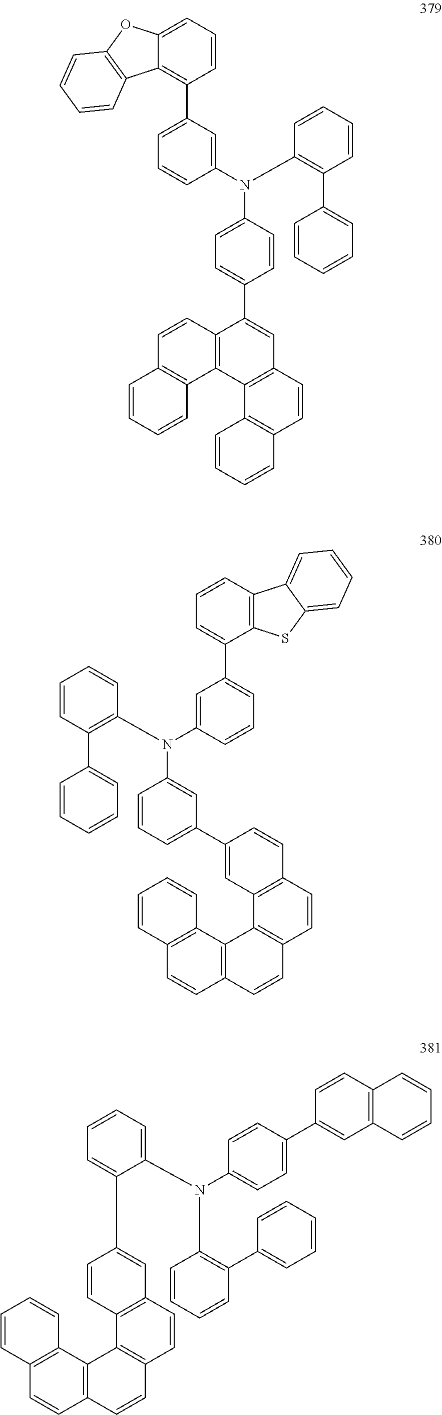 [SMILES: c1ccc(-c2ccccc2N(c2ccc(-c3cc4ccc5ccccc5c4c4c3ccc3ccccc34)cc2)c2cccc(-c3cccc4oc5ccccc5c34)c2)cc1 ; c1ccc(-c2ccccc2N(c2cccc(-c3ccc4ccc5ccc6ccc7ccccc7c6c5c4c3)c2)c2cccc(-c3cccc4c3sc3ccccc34)c2)cc1 ; c1ccc(-c2ccccc2N(c2ccc(-c3ccc4ccccc4c3)cc2)c2ccccc2-c2ccc3c(ccc4ccc5ccc6ccccc6c5c43)c2)cc1]

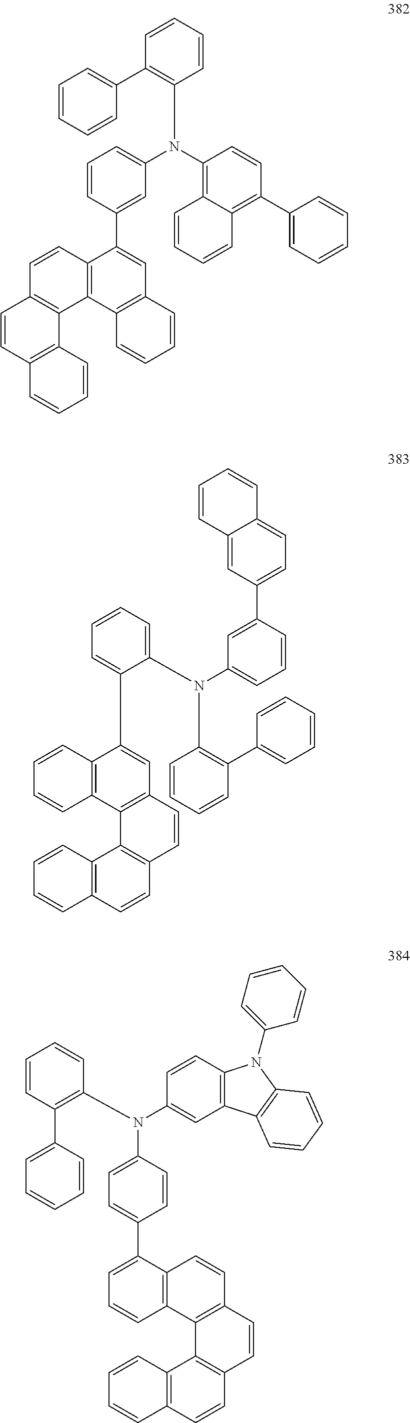 [SMILES: c1ccc(-c2ccccc2N(c2cccc(-c3cc4ccccc4c4c3ccc3ccc5ccccc5c34)c2)c2ccc(-c3ccccc3)c3ccccc23)cc1 ; c1ccc(-c2ccccc2N(c2cccc(-c3ccc4ccccc4c3)c2)c2ccccc2-c2cc3ccc4ccc5ccccc5c4c3c3ccccc23)cc1 ; c1ccc(-c2ccccc2N(c2ccc(-c3cccc4c3ccc3ccc5ccc6ccccc6c5c34)cc2)c2ccc3c(c2)c2ccccc2n3-c2ccccc2)cc1]

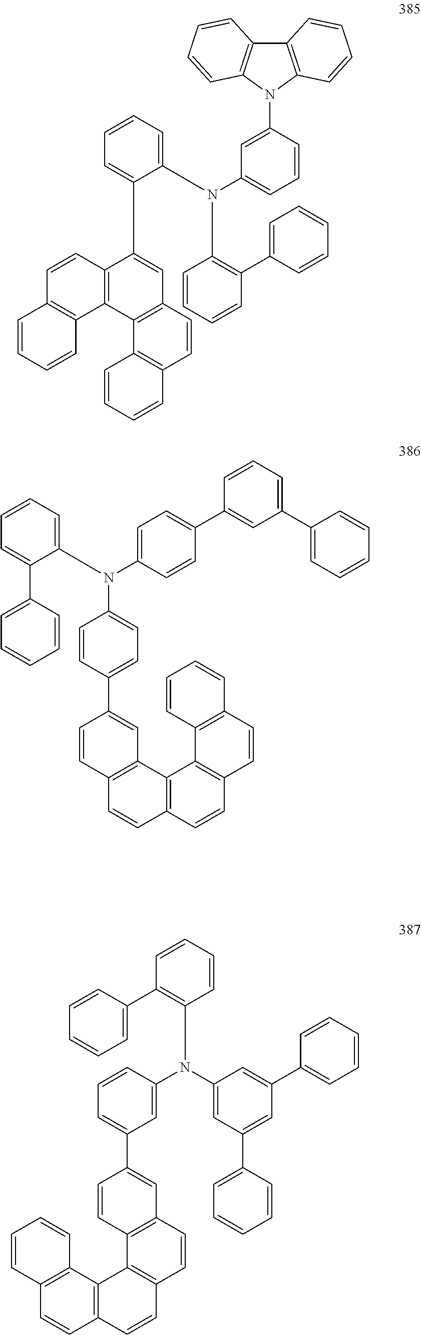 [SMILES: c1ccc(-c2ccccc2N(c2cccc(-n3c4ccccc4c4ccccc43)c2)c2ccccc2-c2cc3ccc4ccccc4c3c3c2ccc2ccccc23)cc1 ; c1ccc(-c2cccc(-c3ccc(N(c4ccc(-c5ccc6ccc7ccc8ccc9ccccc9c8c7c6c5)cc4)c4ccccc4-c4ccccc4)cc3)c2)cc1 ; c1ccc(-c2cc(-c3ccccc3)cc(N(c3cccc(-c4ccc5c(ccc6ccc7ccc8ccccc8c7c65)c4)c3)c3ccccc3-c3ccccc3)c2)cc1]

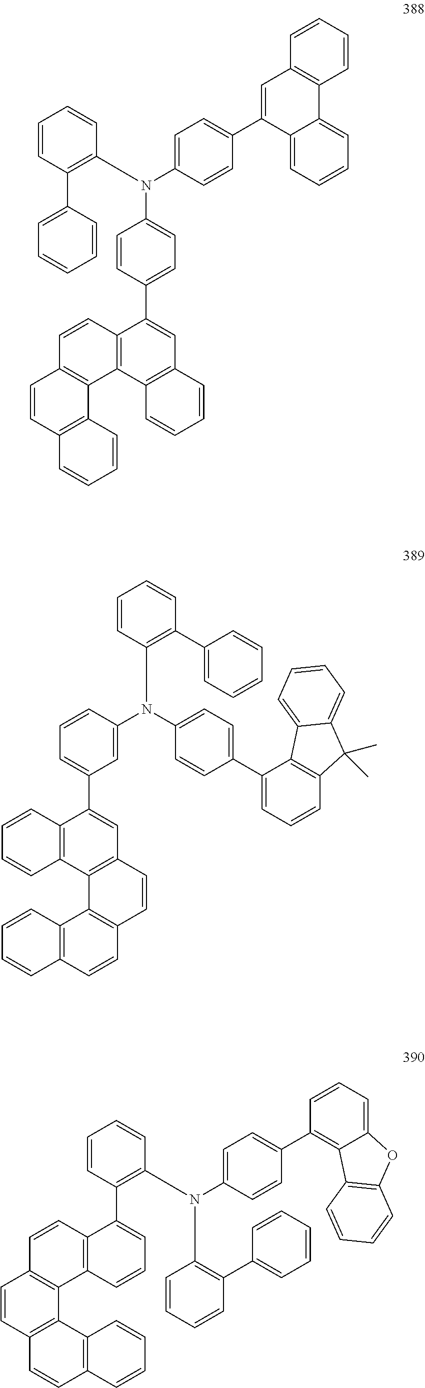 [SMILES: c1ccc(-c2ccccc2N(c2ccc(-c3cc4ccccc4c4ccccc34)cc2)c2ccc(-c3cc4ccccc4c4c3ccc3ccc5ccccc5c34)cc2)cc1 ; CC1(C)c2ccccc2-c2c(-c3ccc(N(c4cccc(-c5cc6ccc7ccc8ccccc8c7c6c6ccccc56)c4)c4ccccc4-c4ccccc4)cc3)cccc21 ; c1ccc(-c2ccccc2N(c2ccc(-c3cccc4oc5ccccc5c34)cc2)c2ccccc2-c2cccc3c2ccc2ccc4ccc5ccccc5c4c23)cc1]

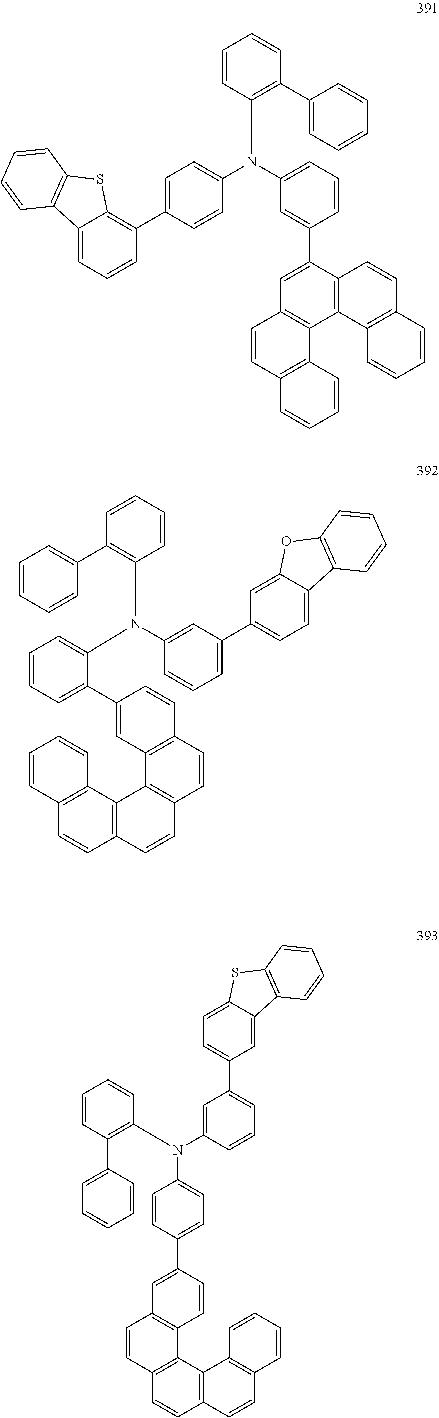 [SMILES: c1ccc(-c2ccccc2N(c2ccc(-c3cccc4c3sc3ccccc34)cc2)c2cccc(-c3cc4ccc5ccccc5c4c4c3ccc3ccccc34)c2)cc1 ; c1ccc(-c2ccccc2N(c2cccc(-c3ccc4c(c3)oc3ccccc34)c2)c2ccccc2-c2ccc3ccc4ccc5ccc6ccccc6c5c4c3c2)cc1 ; c1ccc(-c2ccccc2N(c2ccc(-c3ccc4c(ccc5ccc6ccc7ccccc7c6c54)c3)cc2)c2cccc(-c3ccc4sc5ccccc5c4c3)c2)cc1]

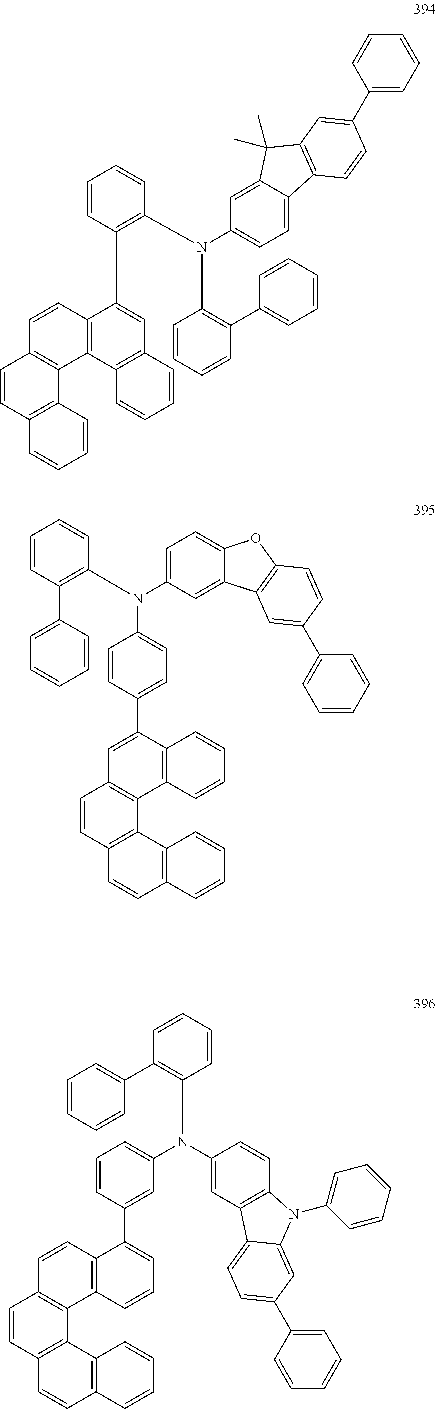 [SMILES: CC1(C)c2cc(-c3ccccc3)ccc2-c2ccc(N(c3ccccc3-c3ccccc3)c3ccccc3-c3cc4ccccc4c4c3ccc3ccc5ccccc5c34)cc21 ; c1ccc(-c2ccc3oc4ccc(N(c5ccc(-c6cc7ccc8ccc9ccccc9c8c7c7ccccc67)cc5)c5ccccc5-c5ccccc5)cc4c3c2)cc1 ; c1ccc(-c2ccc3c4cc(N(c5cccc(-c6cccc7c6ccc6ccc8ccc9ccccc9c8c67)c5)c5ccccc5-c5ccccc5)ccc4n(-c4ccccc4)c3c2)cc1]

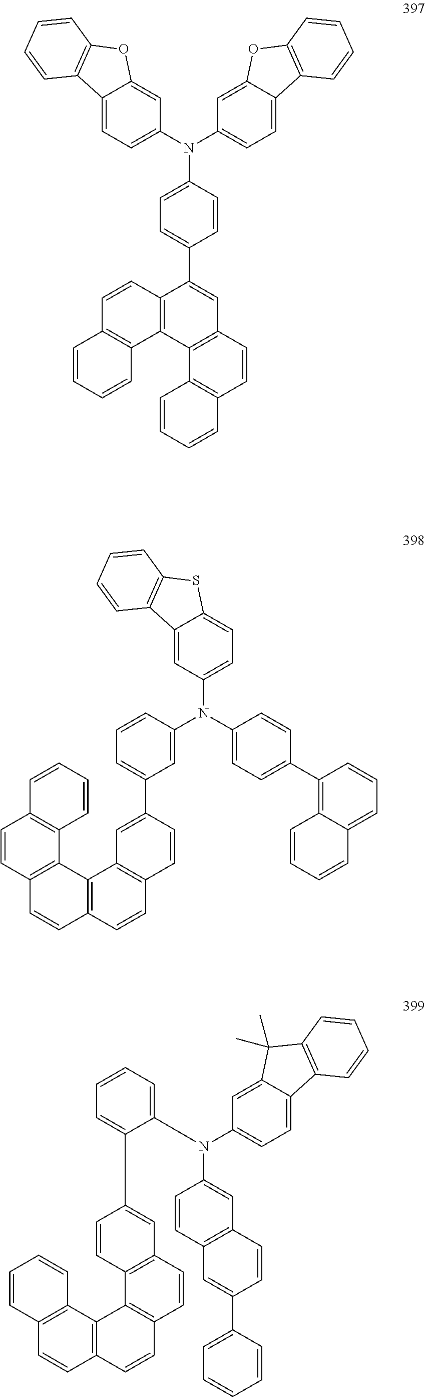 [SMILES: c1ccc2c(c1)ccc1cc(-c3ccc(N(c4ccc5c(c4)oc4ccccc45)c4ccc5c(c4)oc4ccccc45)cc3)c3ccc4ccccc4c3c12 ; c1cc(-c2ccc3ccc4ccc5ccc6ccccc6c5c4c3c2)cc(N(c2ccc(-c3cccc4ccccc34)cc2)c2ccc3sc4ccccc4c3c2)c1 ; CC1(C)c2ccccc2-c2ccc(N(c3ccc4cc(-c5ccccc5)ccc4c3)c3ccccc3-c3ccc4c(ccc5ccc6ccc7ccccc7c6c54)c3)cc21]

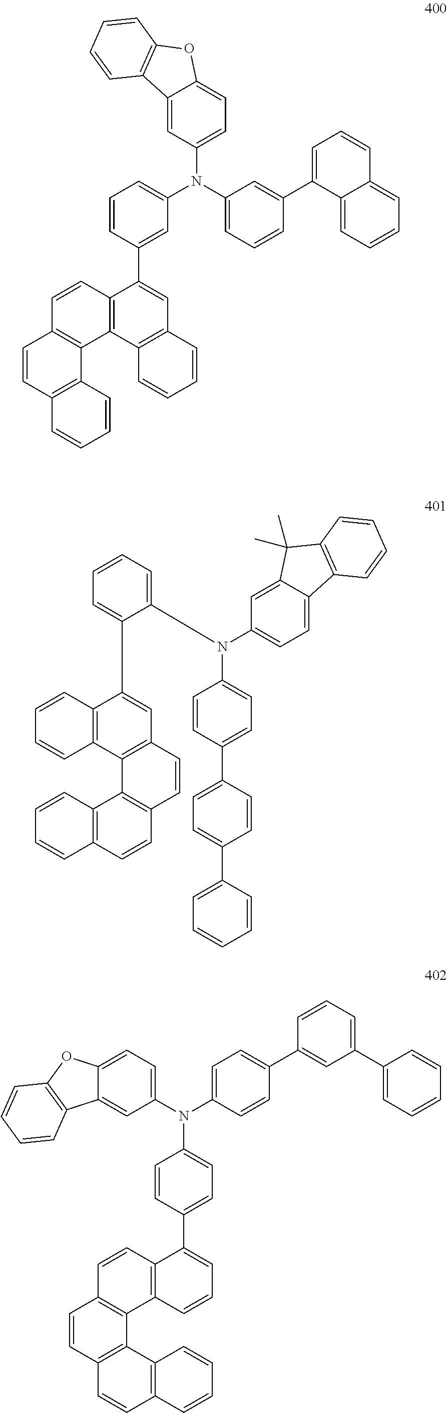 [SMILES: c1cc(-c2cccc3ccccc23)cc(N(c2cccc(-c3cc4ccccc4c4c3ccc3ccc5ccccc5c34)c2)c2ccc3oc4ccccc4c3c2)c1 ; CC1(C)c2ccccc2-c2ccc(N(c3ccc(-c4ccc(-c5ccccc5)cc4)cc3)c3ccccc3-c3cc4ccc5ccc6ccccc6c5c4c4ccccc34)cc21 ; c1ccc(-c2cccc(-c3ccc(N(c4ccc(-c5cccc6c5ccc5ccc7ccc8ccccc8c7c56)cc4)c4ccc5oc6ccccc6c5c4)cc3)c2)cc1]

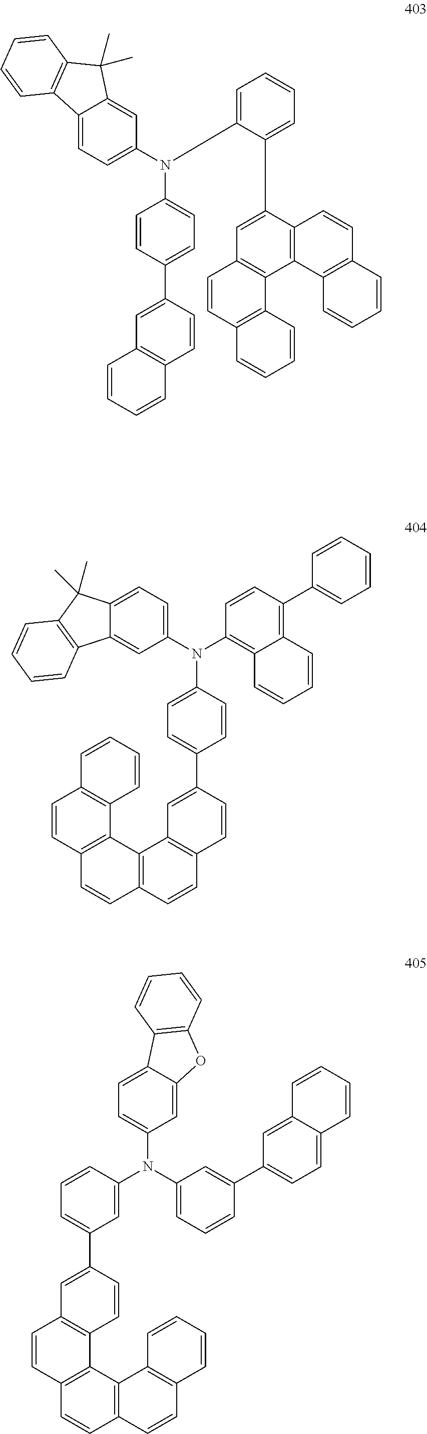 [SMILES: CC1(C)c2ccccc2-c2ccc(N(c3ccc(-c4ccc5ccccc5c4)cc3)c3ccccc3-c3cc4ccc5ccccc5c4c4c3ccc3ccccc34)cc21 ; CC1(C)c2ccccc2-c2cc(N(c3ccc(-c4ccc5ccc6ccc7ccc8ccccc8c7c6c5c4)cc3)c3ccc(-c4ccccc4)c4ccccc34)ccc21 ; c1cc(-c2ccc3ccccc3c2)cc(N(c2cccc(-c3ccc4c(ccc5ccc6ccc7ccccc7c6c54)c3)c2)c2ccc3c(c2)oc2ccccc23)c1]

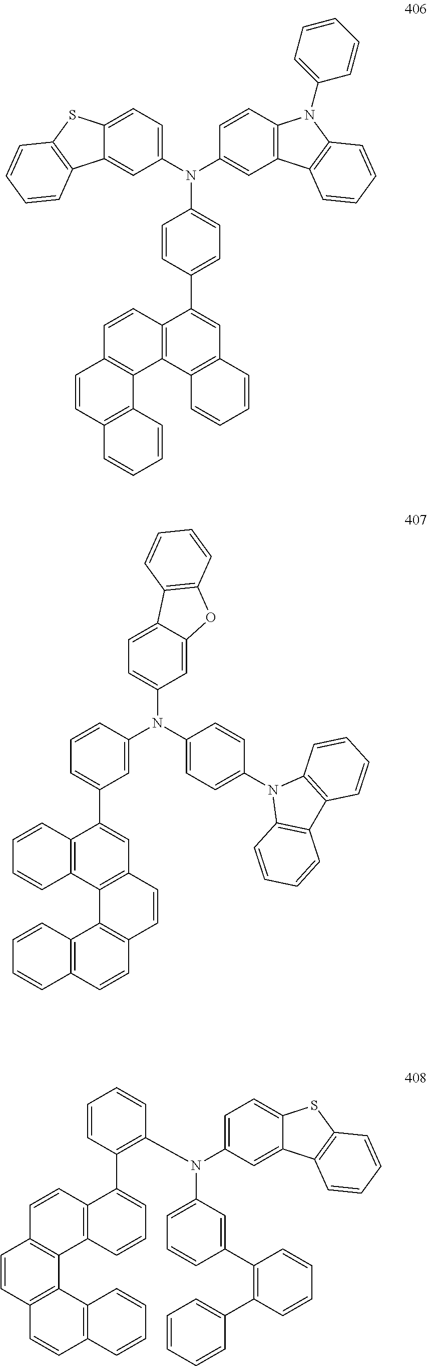 [SMILES: c1ccc(-n2c3ccccc3c3cc(N(c4ccc(-c5cc6ccccc6c6c5ccc5ccc7ccccc7c56)cc4)c4ccc5sc6ccccc6c5c4)ccc32)cc1 ; c1cc(-c2cc3ccc4ccc5ccccc5c4c3c3ccccc23)cc(N(c2ccc(-n3c4ccccc4c4ccccc43)cc2)c2ccc3c(c2)oc2ccccc23)c1 ; c1ccc(-c2ccccc2-c2cccc(N(c3ccc4sc5ccccc5c4c3)c3ccccc3-c3cccc4c3ccc3ccc5ccc6ccccc6c5c34)c2)cc1]

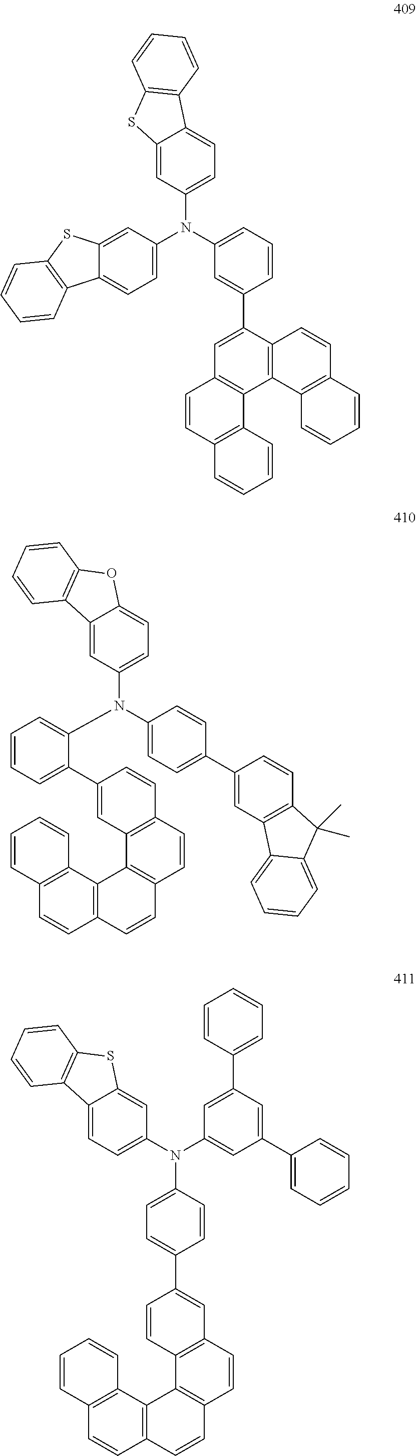 [SMILES: c1cc(-c2cc3ccc4ccccc4c3c3c2ccc2ccccc23)cc(N(c2ccc3c(c2)sc2ccccc23)c2ccc3c(c2)sc2ccccc23)c1 ; CC1(C)c2ccccc2-c2cc(-c3ccc(N(c4ccc5oc6ccccc6c5c4)c4ccccc4-c4ccc5ccc6ccc7ccc8ccccc8c7c6c5c4)cc3)ccc21 ; c1ccc(-c2cc(-c3ccccc3)cc(N(c3ccc(-c4ccc5c(ccc6ccc7ccc8ccccc8c7c65)c4)cc3)c3ccc4c(c3)sc3ccccc34)c2)cc1]

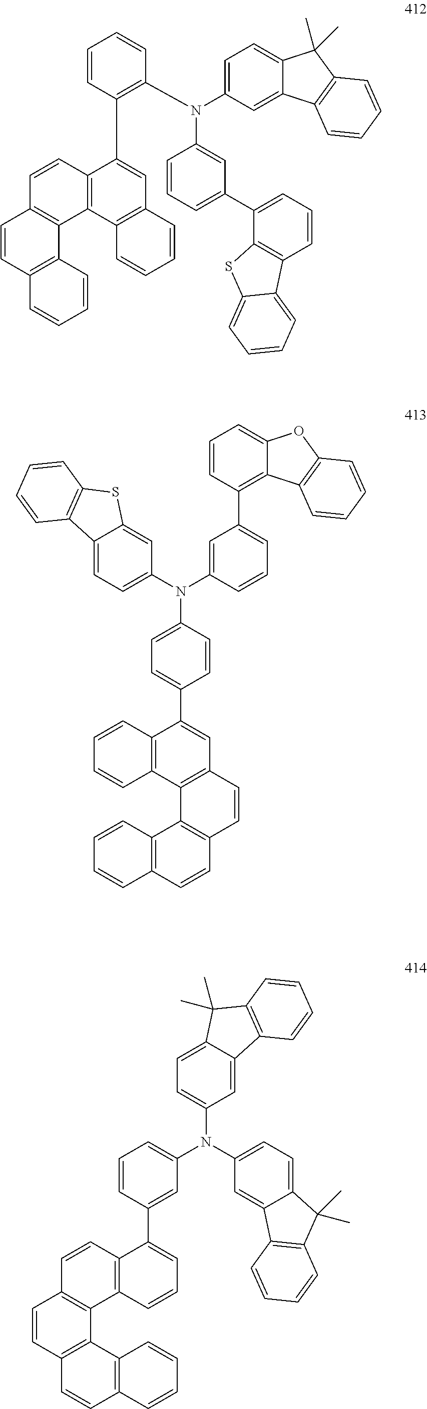 [SMILES: CC1(C)c2ccccc2-c2cc(N(c3cccc(-c4cccc5c4sc4ccccc45)c3)c3ccccc3-c3cc4ccccc4c4c3ccc3ccc5ccccc5c34)ccc21 ; c1cc(-c2cccc3oc4ccccc4c23)cc(N(c2ccc(-c3cc4ccc5ccc6ccccc6c5c4c4ccccc34)cc2)c2ccc3c(c2)sc2ccccc23)c1 ; CC1(C)c2ccccc2-c2cc(N(c3cccc(-c4cccc5c4ccc4ccc6ccc7ccccc7c6c45)c3)c3ccc4c(c3)-c3ccccc3C4(C)C)ccc21]

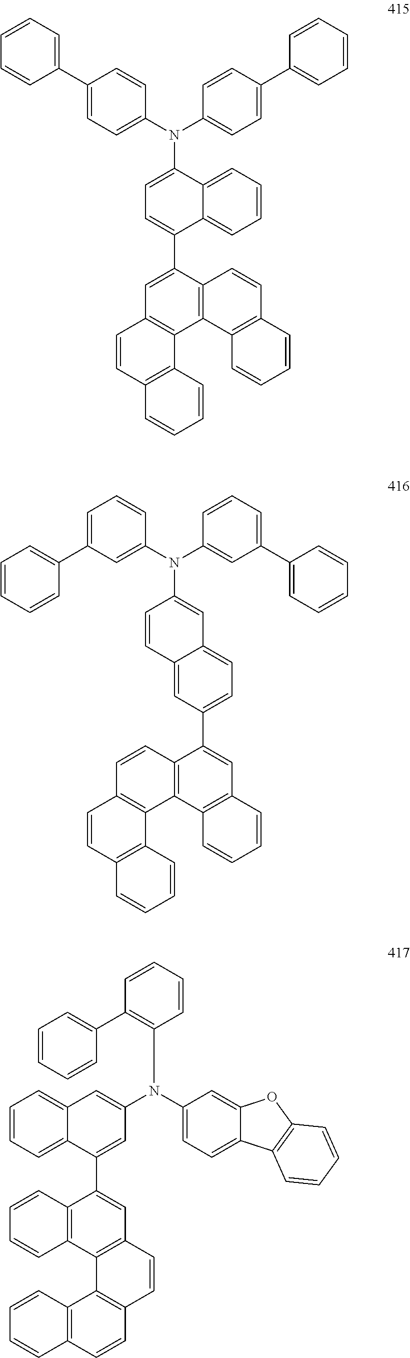 [SMILES: c1ccc(-c2ccc(N(c3ccc(-c4ccccc4)cc3)c3ccc(-c4cc5ccc6ccccc6c5c5c4ccc4ccccc45)c4ccccc34)cc2)cc1 ; c1ccc(-c2cccc(N(c3cccc(-c4ccccc4)c3)c3ccc4cc(-c5cc6ccccc6c6c5ccc5ccc7ccccc7c56)ccc4c3)c2)cc1 ; c1ccc(-c2ccccc2N(c2cc(-c3cc4ccc5ccc6ccccc6c5c4c4ccccc34)c3ccccc3c2)c2ccc3c(c2)oc2ccccc23)cc1]

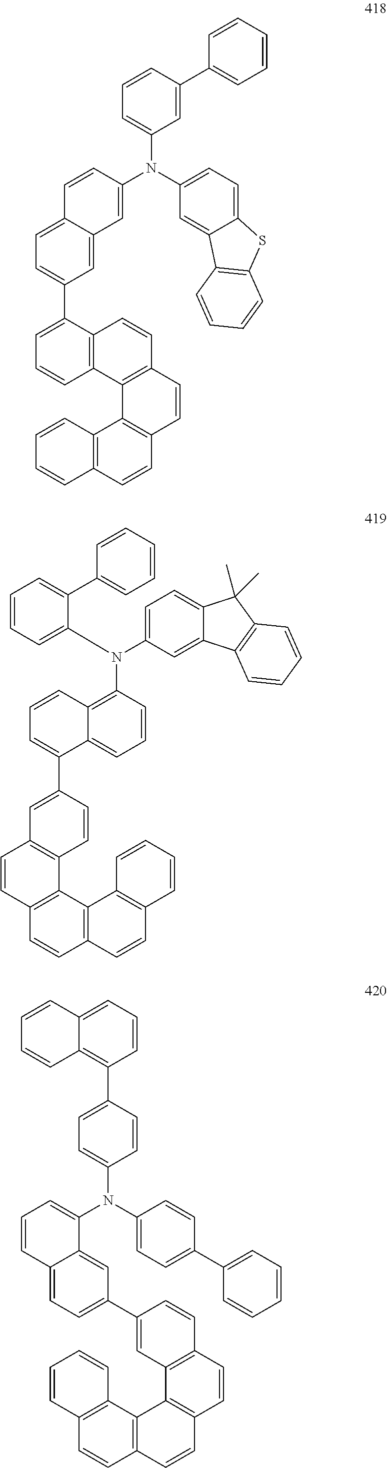

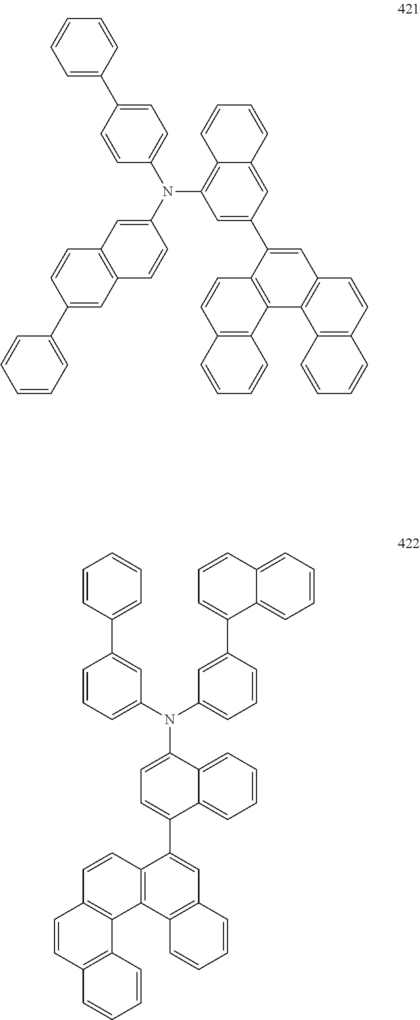 [SMILES: c1ccc(-c2ccc(N(c3ccc4cc(-c5ccccc5)ccc4c3)c3cc(-c4cc5ccc6ccccc6c5c5c4ccc4ccccc45)cc4ccccc34)cc2)cc1 ; c1ccc(-c2cccc(N(c3cccc(-c4cccc5ccccc45)c3)c3ccc(-c4cc5ccccc5c5c4ccc4ccc6ccccc6c45)c4ccccc34)c2)cc1]

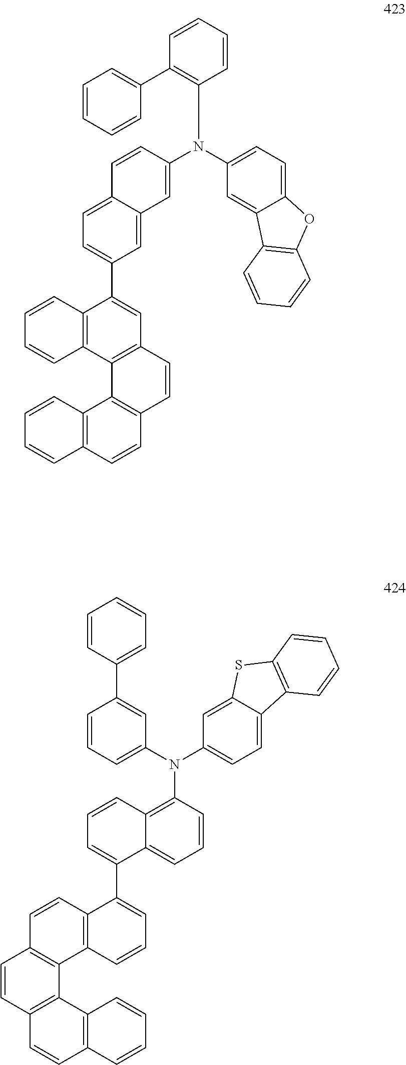 [SMILES: c1ccc(-c2ccccc2N(c2ccc3ccc(-c4cc5ccc6ccc7ccccc7c6c5c5ccccc45)cc3c2)c2ccc3oc4ccccc4c3c2)cc1 ; c1ccc(-c2cccc(N(c3ccc4c(c3)sc3ccccc34)c3cccc4c(-c5cccc6c5ccc5ccc7ccc8ccccc8c7c56)cccc34)c2)cc1]

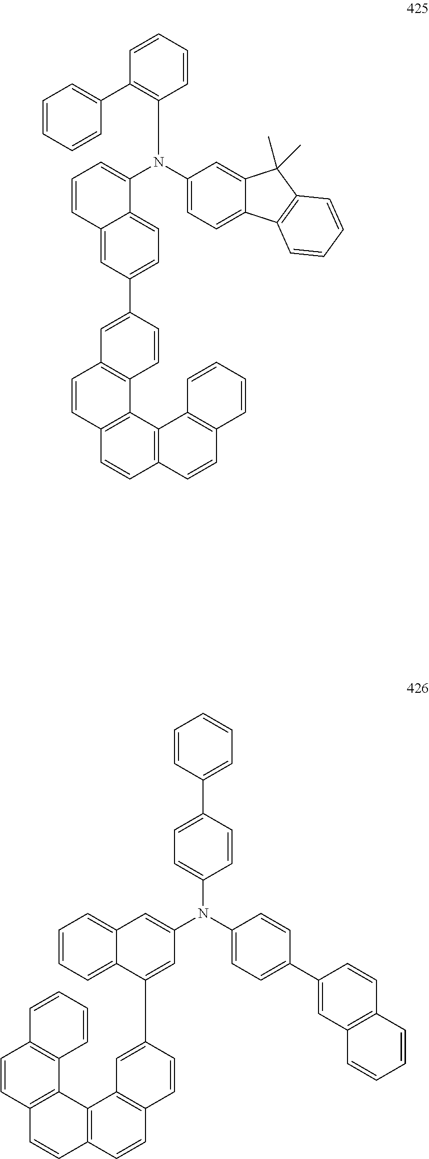 [SMILES: CC1(C)c2ccccc2-c2ccc(N(c3ccccc3-c3ccccc3)c3cccc4cc(-c5ccc6c(ccc7ccc8ccc9ccccc9c8c76)c5)ccc34)cc21 ; c1ccc(-c2ccc(N(c3ccc(-c4ccc5ccccc5c4)cc3)c3cc(-c4ccc5ccc6ccc7ccc8ccccc8c7c6c5c4)c4ccccc4c3)cc2)cc1]

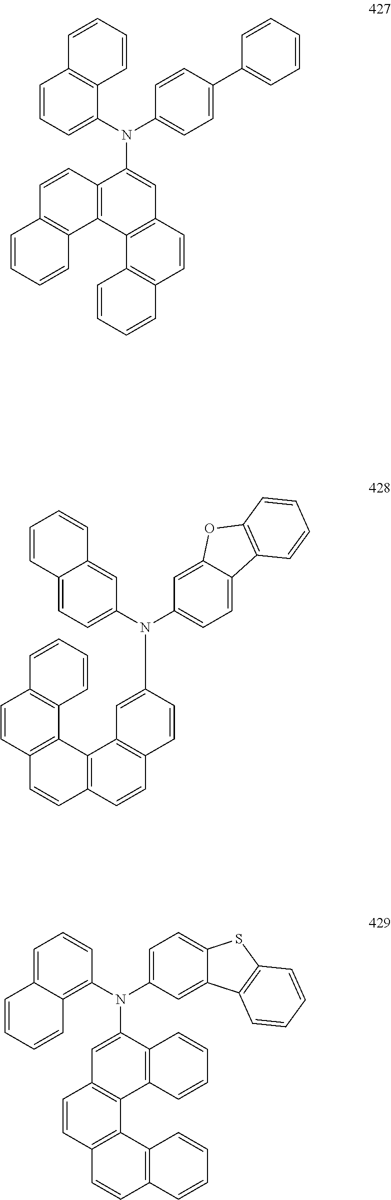 [SMILES: c1ccc(-c2ccc(N(c3cccc4ccccc34)c3cc4ccc5ccccc5c4c4c3ccc3ccccc34)cc2)cc1 ; c1ccc2cc(N(c3ccc4c(c3)oc3ccccc34)c3ccc4ccc5ccc6ccc7ccccc7c6c5c4c3)ccc2c1 ; c1ccc2c(N(c3ccc4sc5ccccc5c4c3)c3cc4ccc5ccc6ccccc6c5c4c4ccccc34)cccc2c1]

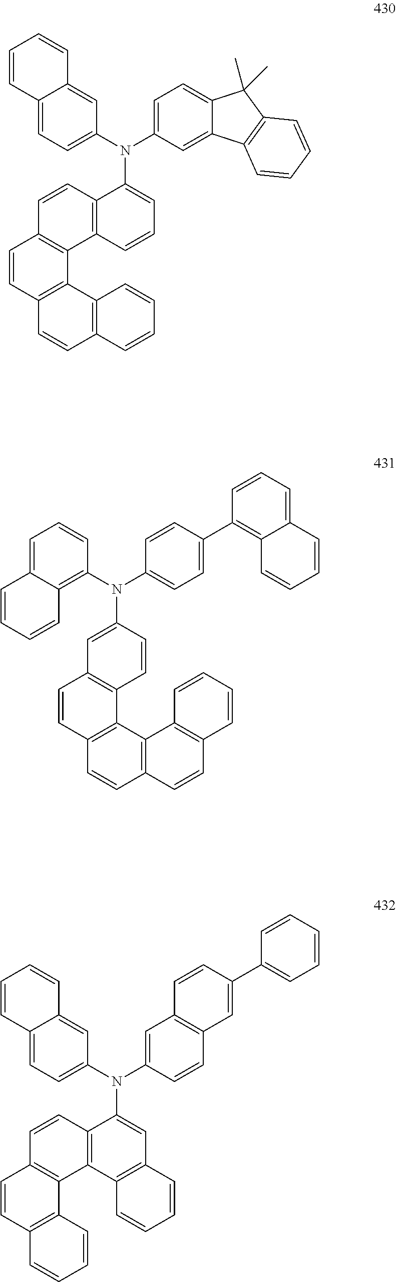 [SMILES: CC1(C)c2ccccc2-c2cc(N(c3ccc4ccccc4c3)c3cccc4c3ccc3ccc5ccc6ccccc6c5c34)ccc21 ; c1ccc2c(-c3ccc(N(c4ccc5c(ccc6ccc7ccc8ccccc8c7c65)c4)c4cccc5ccccc45)cc3)cccc2c1 ; c1ccc(-c2ccc3cc(N(c4ccc5ccccc5c4)c4cc5ccccc5c5c4ccc4ccc6ccccc6c45)ccc3c2)cc1]

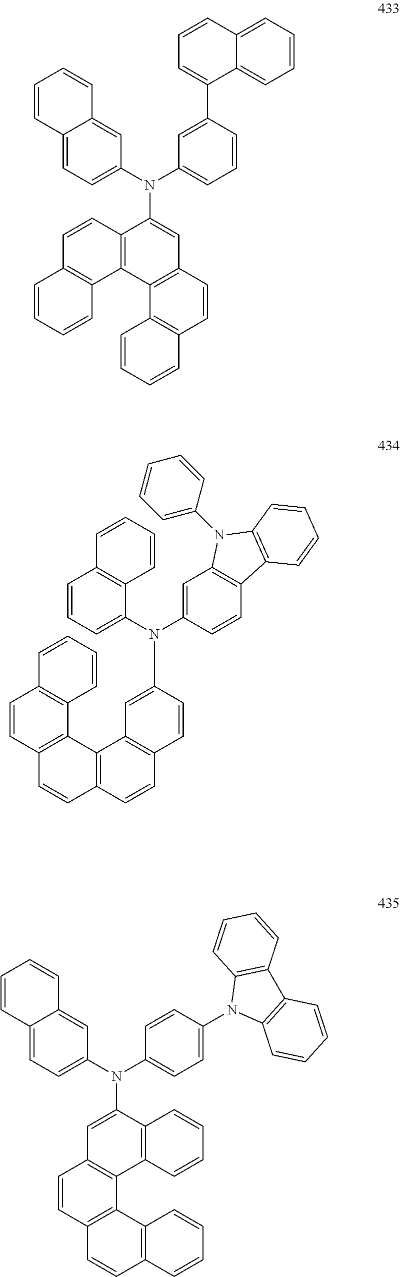 [SMILES: c1cc(-c2cccc3ccccc23)cc(N(c2ccc3ccccc3c2)c2cc3ccc4ccccc4c3c3c2ccc2ccccc23)c1 ; c1ccc(-n2c3ccccc3c3ccc(N(c4ccc5ccc6ccc7ccc8ccccc8c7c6c5c4)c4cccc5ccccc45)cc32)cc1 ; c1ccc2cc(N(c3ccc(-n4c5ccccc5c5ccccc54)cc3)c3cc4ccc5ccc6ccccc6c5c4c4ccccc34)ccc2c1]

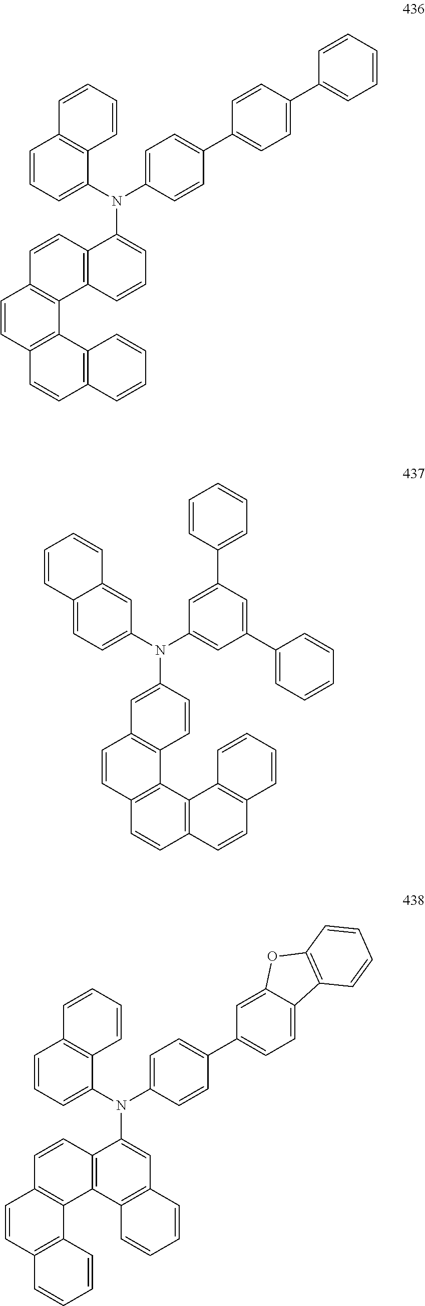 [SMILES: c1ccc(-c2ccc(-c3ccc(N(c4cccc5ccccc45)c4cccc5c4ccc4ccc6ccc7ccccc7c6c45)cc3)cc2)cc1 ; c1ccc(-c2cc(-c3ccccc3)cc(N(c3ccc4ccccc4c3)c3ccc4c(ccc5ccc6ccc7ccccc7c6c54)c3)c2)cc1 ; c1ccc2c(N(c3ccc(-c4ccc5c(c4)oc4ccccc45)cc3)c3cc4ccccc4c4c3ccc3ccc5ccccc5c34)cccc2c1]

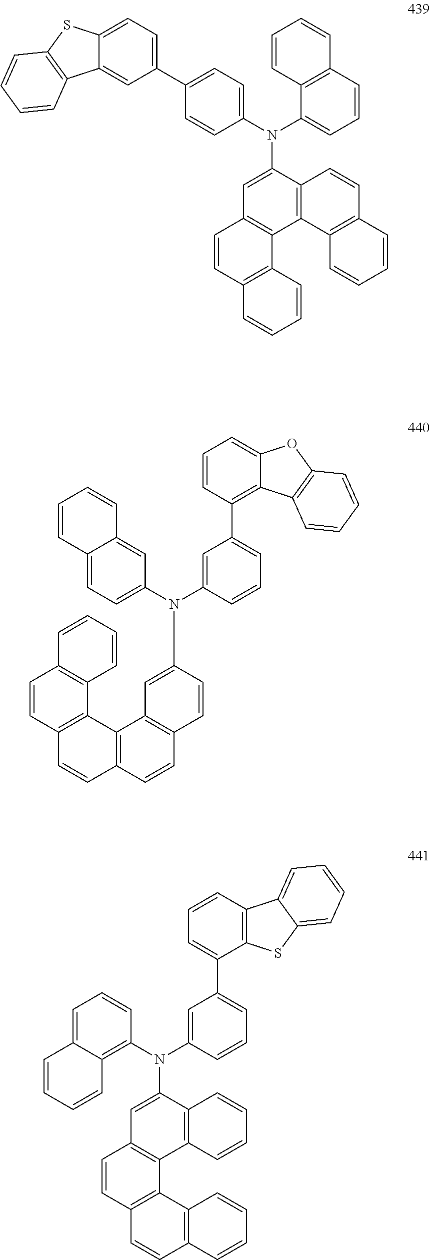 [SMILES: c1ccc2c(N(c3ccc(-c4ccc5sc6ccccc6c5c4)cc3)c3cc4ccc5ccccc5c4c4c3ccc3ccccc34)cccc2c1 ; c1cc(-c2cccc3oc4ccccc4c23)cc(N(c2ccc3ccccc3c2)c2ccc3ccc4ccc5ccc6ccccc6c5c4c3c2)c1 ; c1cc(-c2cccc3c2sc2ccccc23)cc(N(c2cccc3ccccc23)c2cc3ccc4ccc5ccccc5c4c3c3ccccc23)c1]

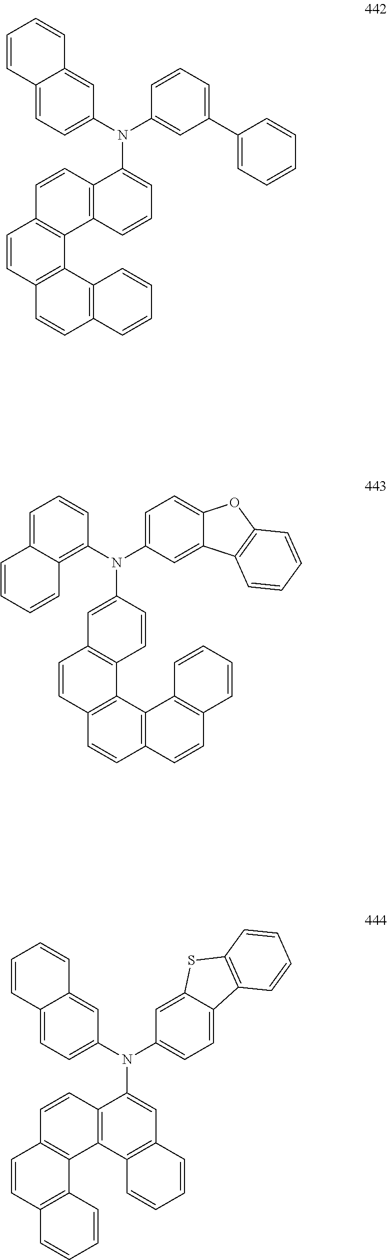 [SMILES: c1ccc(-c2cccc(N(c3ccc4ccccc4c3)c3cccc4c3ccc3ccc5ccc6ccccc6c5c34)c2)cc1 ; c1ccc2c(N(c3ccc4c(ccc5ccc6ccc7ccccc7c6c54)c3)c3ccc4oc5ccccc5c4c3)cccc2c1 ; c1ccc2cc(N(c3ccc4c(c3)sc3ccccc34)c3cc4ccccc4c4c3ccc3ccc5ccccc5c34)ccc2c1]

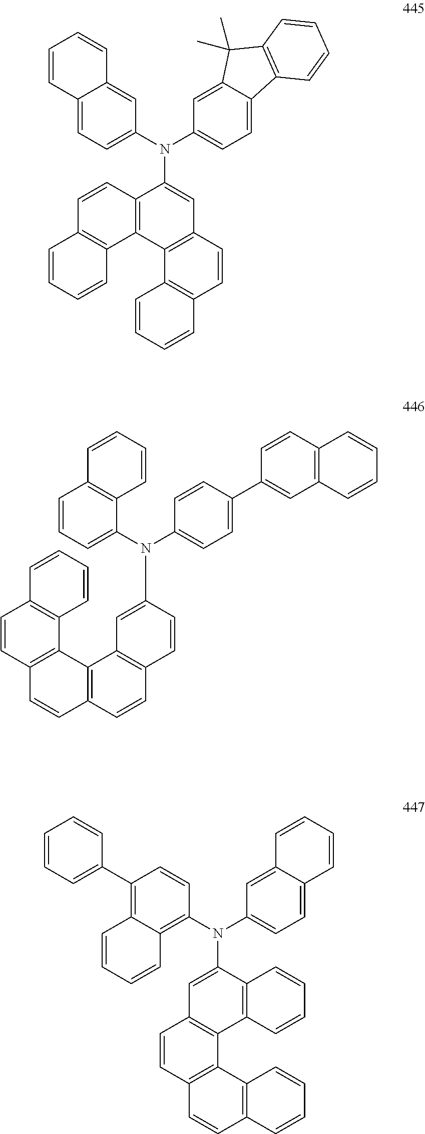 [SMILES: CC1(C)c2ccccc2-c2ccc(N(c3ccc4ccccc4c3)c3cc4ccc5ccccc5c4c4c3ccc3ccccc34)cc21 ; c1ccc2cc(-c3ccc(N(c4ccc5ccc6ccc7ccc8ccccc8c7c6c5c4)c4cccc5ccccc45)cc3)ccc2c1 ; c1ccc(-c2ccc(N(c3ccc4ccccc4c3)c3cc4ccc5ccc6ccccc6c5c4c4ccccc34)c3ccccc23)cc1]

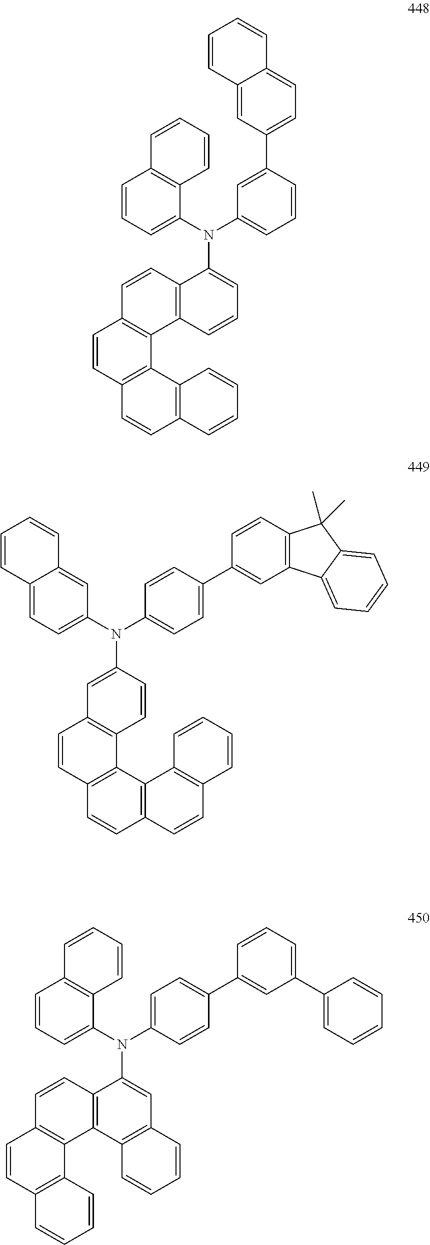 [SMILES: c1cc(-c2ccc3ccccc3c2)cc(N(c2cccc3ccccc23)c2cccc3c2ccc2ccc4ccc5ccccc5c4c23)c1 ; CC1(C)c2ccccc2-c2cc(-c3ccc(N(c4ccc5ccccc5c4)c4ccc5c(ccc6ccc7ccc8ccccc8c7c65)c4)cc3)ccc21 ; c1ccc(-c2cccc(-c3ccc(N(c4cccc5ccccc45)c4cc5ccccc5c5c4ccc4ccc6ccccc6c45)cc3)c2)cc1]

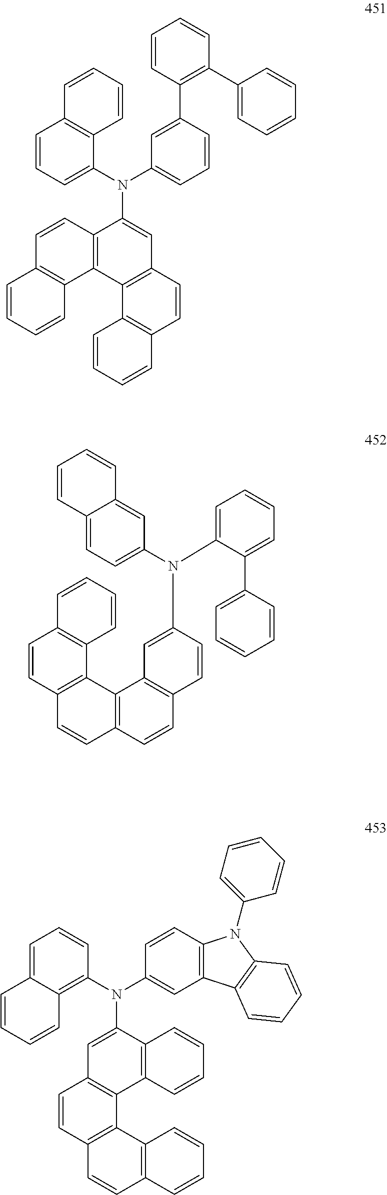 [SMILES: c1ccc(-c2ccccc2-c2cccc(N(c3cccc4ccccc34)c3cc4ccc5ccccc5c4c4c3ccc3ccccc34)c2)cc1 ; c1ccc(-c2ccccc2N(c2ccc3ccccc3c2)c2ccc3ccc4ccc5ccc6ccccc6c5c4c3c2)cc1 ; c1ccc(-n2c3ccccc3c3cc(N(c4cccc5ccccc45)c4cc5ccc6ccc7ccccc7c6c5c5ccccc45)ccc32)cc1]

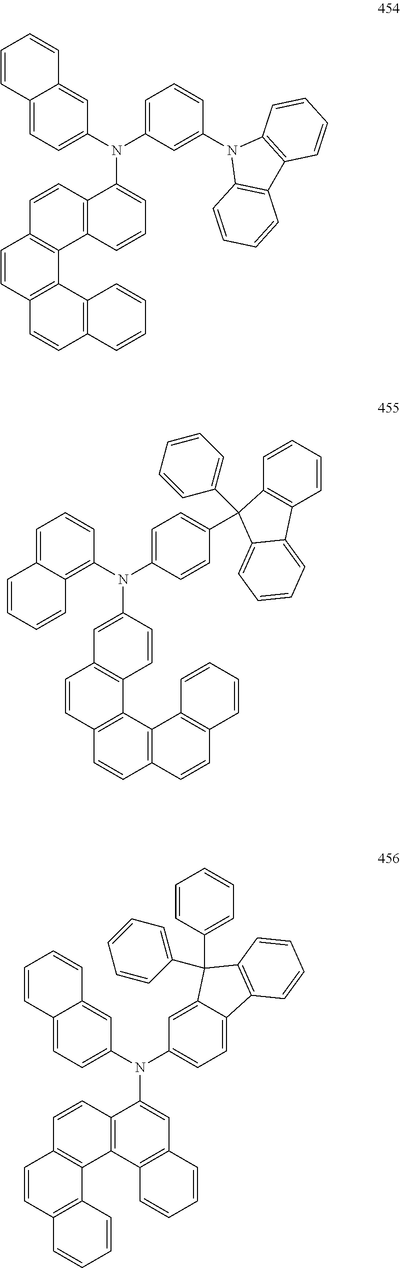 [SMILES: c1cc(N(c2ccc3ccccc3c2)c2cccc3c2ccc2ccc4ccc5ccccc5c4c23)cc(-n2c3ccccc3c3ccccc32)c1 ; c1ccc(C2(c3ccc(N(c4ccc5c(ccc6ccc7ccc8ccccc8c7c65)c4)c4cccc5ccccc45)cc3)c3ccccc3-c3ccccc32)cc1 ; c1ccc(C2(c3ccccc3)c3ccccc3-c3ccc(N(c4ccc5ccccc5c4)c4cc5ccccc5c5c4ccc4ccc6ccccc6c45)cc32)cc1]

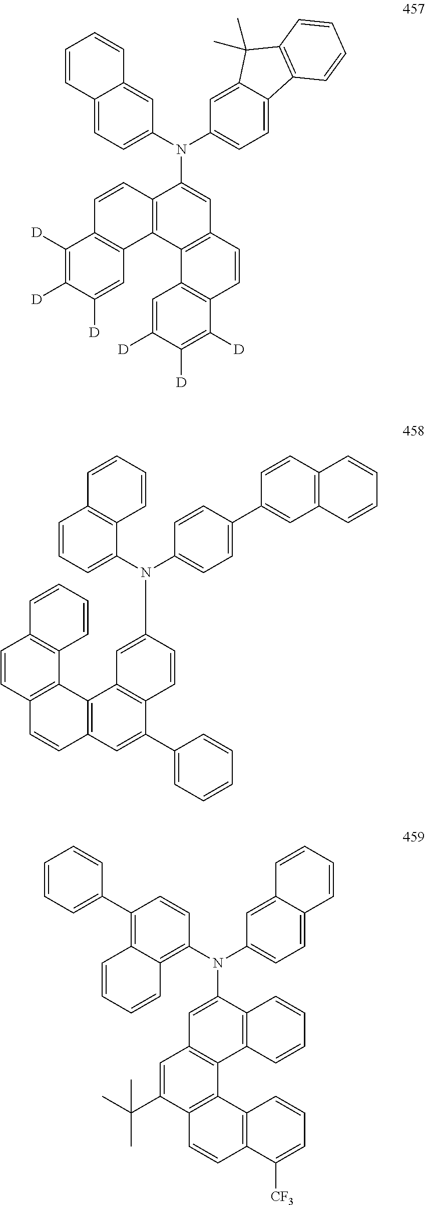 [SMILES: [2H]c1cc2c(ccc3cc(N(c4ccc5c(c4)C(C)(C)c4ccccc4-5)c4ccc5ccccc5c4)c4ccc5c([2H])c([2H])c([2H])cc5c4c32)c([2H])c1[2H] ; c1ccc(-c2cc3ccc4ccc5ccccc5c4c3c3cc(N(c4ccc(-c5ccc6ccccc6c5)cc4)c4cccc5ccccc45)ccc23)cc1 ; CC(C)(C)c1cc2cc(N(c3ccc4ccccc4c3)c3ccc(-c4ccccc4)c4ccccc34)c3ccccc3c2c2c1ccc1c(C(F)(F)F)cccc12]

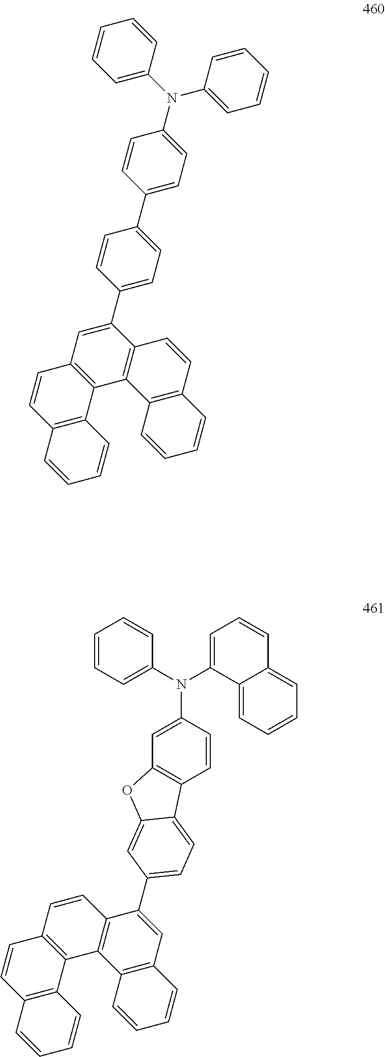 [SMILES: c1ccc(N(c2ccccc2)c2ccc(-c3ccc(-c4cc5ccc6ccccc6c5c5c4ccc4ccccc45)cc3)cc2)cc1 ; c1ccc(N(c2ccc3c(c2)oc2cc(-c4cc5ccccc5c5c4ccc4ccc6ccccc6c45)ccc23)c2cccc3ccccc23)cc1]

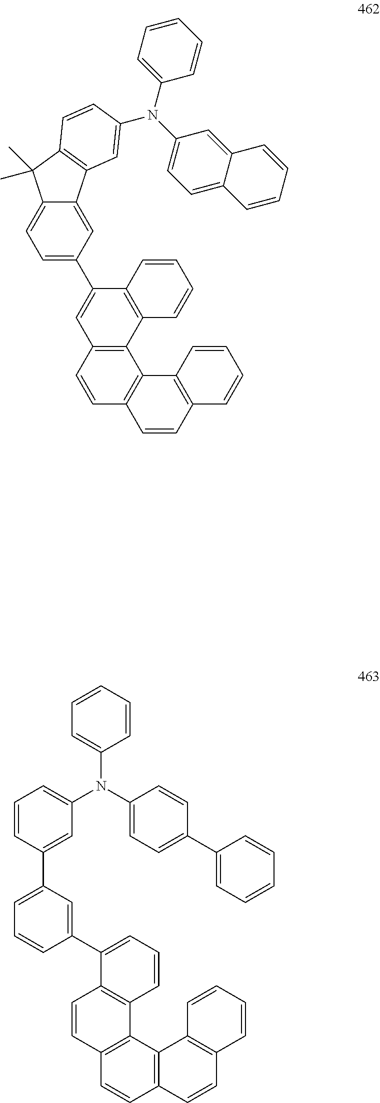 [SMILES: CC1(C)c2ccc(-c3cc4ccc5ccc6ccccc6c5c4c4ccccc34)cc2-c2cc(N(c3ccccc3)c3ccc4ccccc4c3)ccc21 ; c1ccc(-c2ccc(N(c3ccccc3)c3cccc(-c4cccc(-c5cccc6c5ccc5ccc7ccc8ccccc8c7c56)c4)c3)cc2)cc1]

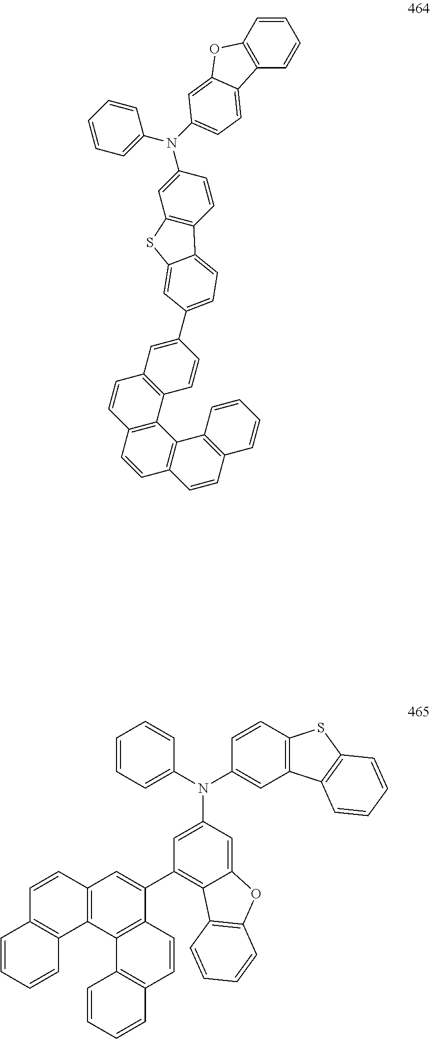 [SMILES: c1ccc(N(c2ccc3c(c2)oc2ccccc23)c2ccc3c(c2)sc2cc(-c4ccc5c(ccc6ccc7ccc8ccccc8c7c65)c4)ccc23)cc1 ; c1ccc(N(c2cc(-c3cc4ccc5ccccc5c4c4c3ccc3ccccc34)c3c(c2)oc2ccccc23)c2ccc3sc4ccccc4c3c2)cc1]

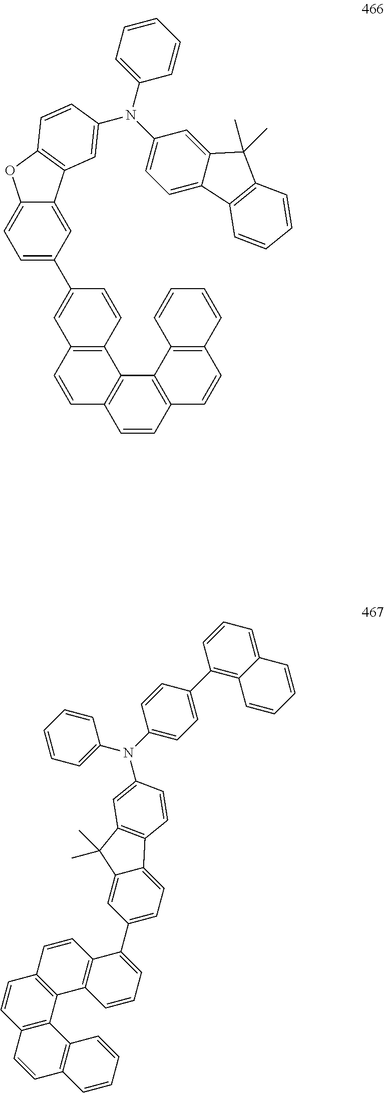 [SMILES: CC1(C)c2ccccc2-c2ccc(N(c3ccccc3)c3ccc4oc5ccc(-c6ccc7c(ccc8ccc9ccc%10ccccc%10c9c87)c6)cc5c4c3)cc21 ; CC1(C)c2cc(-c3cccc4c3ccc3ccc5ccc6ccccc6c5c34)ccc2-c2ccc(N(c3ccccc3)c3ccc(-c4cccc5ccccc45)cc3)cc21]

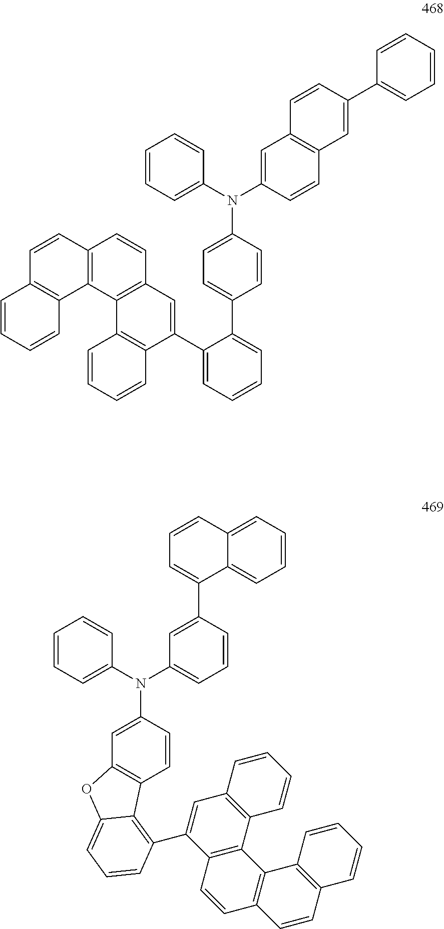 [SMILES: c1ccc(-c2ccc3cc(N(c4ccccc4)c4ccc(-c5ccccc5-c5cc6ccc7ccc8ccccc8c7c6c6ccccc56)cc4)ccc3c2)cc1 ; c1ccc(N(c2cccc(-c3cccc4ccccc34)c2)c2ccc3c(c2)oc2cccc(-c4cc5ccccc5c5c4ccc4ccc6ccccc6c45)c23)cc1]

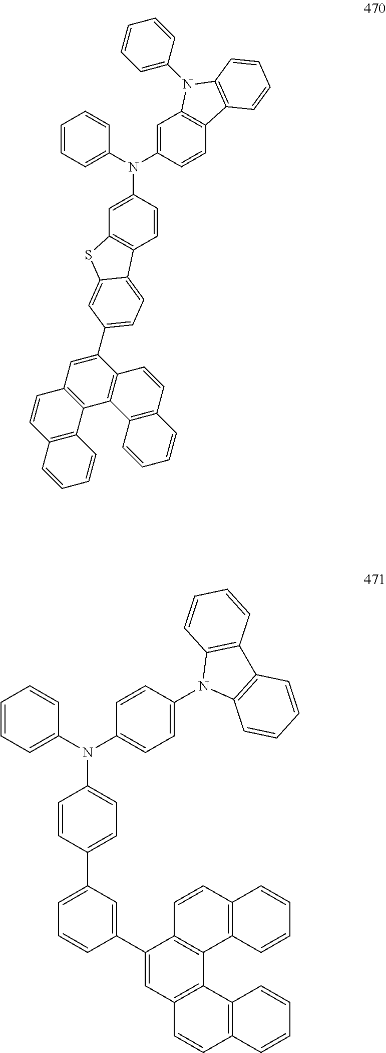 [SMILES: c1ccc(N(c2ccc3c(c2)sc2cc(-c4cc5ccc6ccccc6c5c5c4ccc4ccccc45)ccc23)c2ccc3c4ccccc4n(-c4ccccc4)c3c2)cc1 ; c1ccc(N(c2ccc(-c3cccc(-c4cc5ccc6ccccc6c5c5c4ccc4ccccc45)c3)cc2)c2ccc(-n3c4ccccc4c4ccccc43)cc2)cc1]

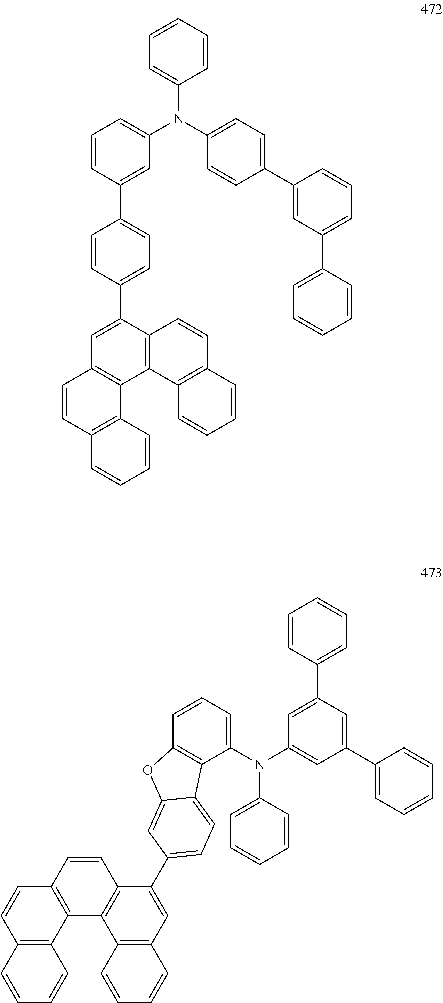 [SMILES: c1ccc(-c2cccc(-c3ccc(N(c4ccccc4)c4cccc(-c5ccc(-c6cc7ccc8ccccc8c7c7c6ccc6ccccc67)cc5)c4)cc3)c2)cc1 ; c1ccc(-c2cc(-c3ccccc3)cc(N(c3ccccc3)c3cccc4oc5cc(-c6cc7ccccc7c7c6ccc6ccc8ccccc8c67)ccc5c34)c2)cc1]

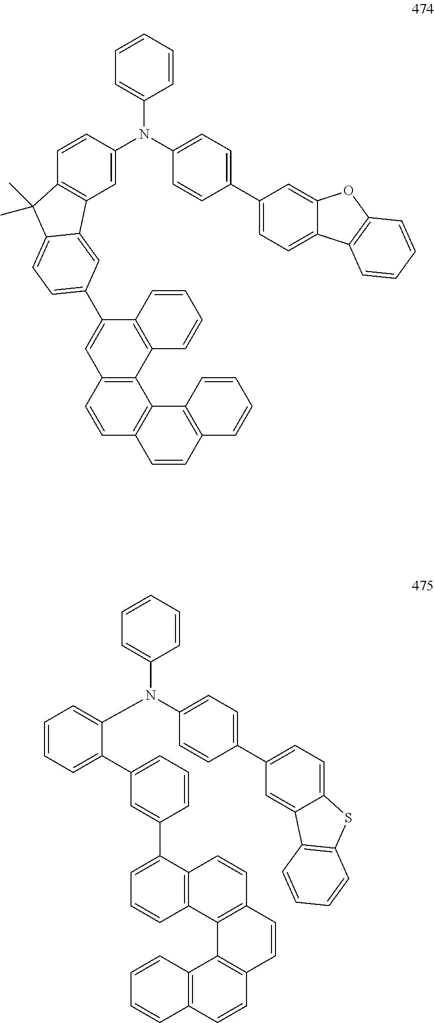 [SMILES: CC1(C)c2ccc(-c3cc4ccc5ccc6ccccc6c5c4c4ccccc34)cc2-c2cc(N(c3ccccc3)c3ccc(-c4ccc5c(c4)oc4ccccc45)cc3)ccc21 ; c1ccc(N(c2ccc(-c3ccc4sc5ccccc5c4c3)cc2)c2ccccc2-c2cccc(-c3cccc4c3ccc3ccc5ccc6ccccc6c5c34)c2)cc1]

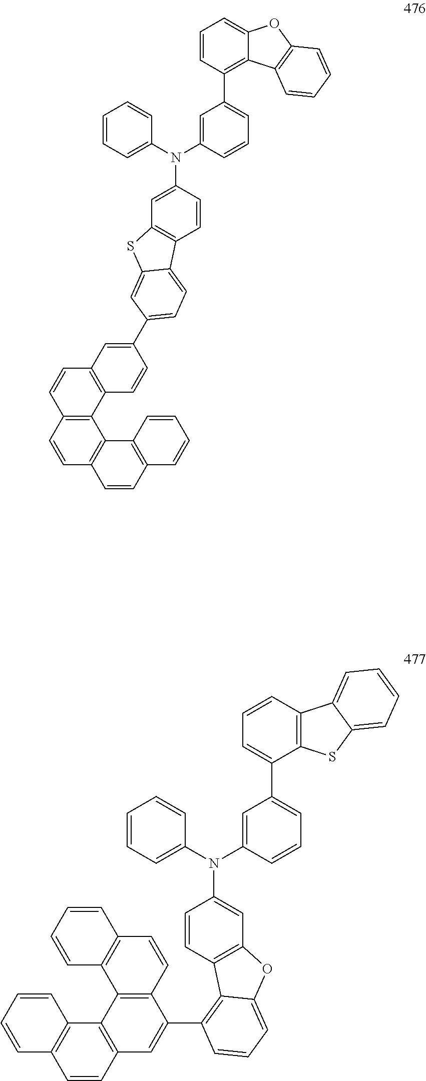 [SMILES: c1ccc(N(c2cccc(-c3cccc4oc5ccccc5c34)c2)c2ccc3c(c2)sc2cc(-c4ccc5c(ccc6ccc7ccc8ccccc8c7c65)c4)ccc23)cc1 ; c1ccc(N(c2cccc(-c3cccc4c3sc3ccccc34)c2)c2ccc3c(c2)oc2cccc(-c4cc5ccc6ccccc6c5c5c4ccc4ccccc45)c23)cc1]

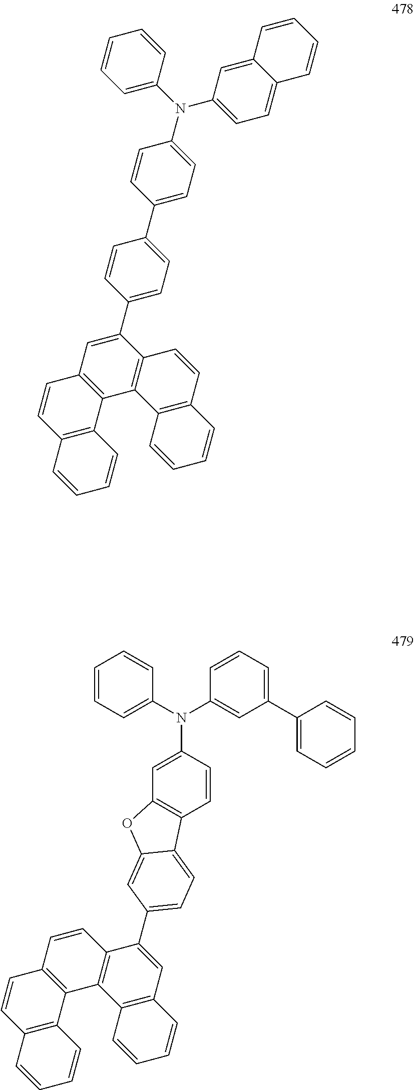 [SMILES: c1ccc(N(c2ccc(-c3ccc(-c4cc5ccc6ccccc6c5c5c4ccc4ccccc45)cc3)cc2)c2ccc3ccccc3c2)cc1 ; c1ccc(-c2cccc(N(c3ccccc3)c3ccc4c(c3)oc3cc(-c5cc6ccccc6c6c5ccc5ccc7ccccc7c56)ccc34)c2)cc1]

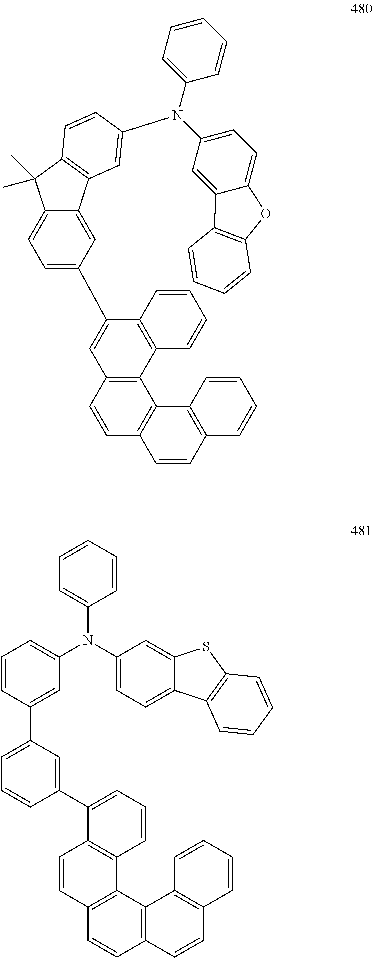 [SMILES: CC1(C)c2ccc(-c3cc4ccc5ccc6ccccc6c5c4c4ccccc34)cc2-c2cc(N(c3ccccc3)c3ccc4oc5ccccc5c4c3)ccc21 ; c1ccc(N(c2cccc(-c3cccc(-c4cccc5c4ccc4ccc6ccc7ccccc7c6c45)c3)c2)c2ccc3c(c2)sc2ccccc23)cc1]

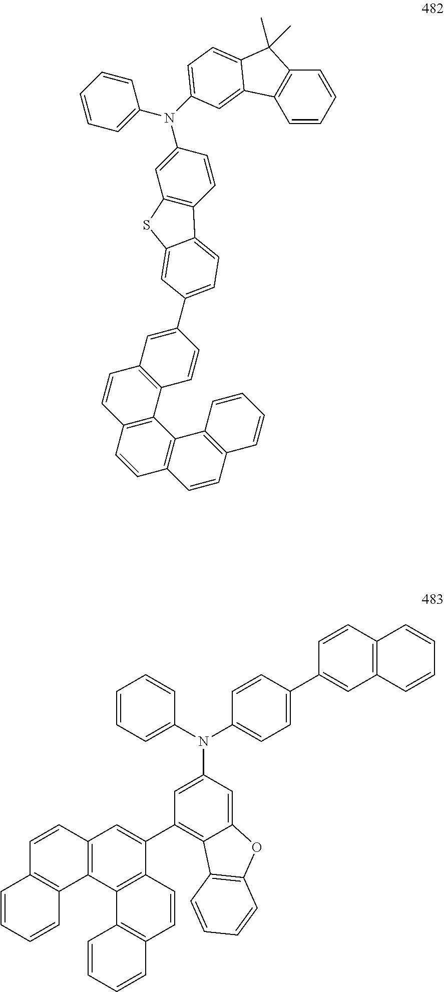 [SMILES: CC1(C)c2ccccc2-c2cc(N(c3ccccc3)c3ccc4c(c3)sc3cc(-c5ccc6c(ccc7ccc8ccc9ccccc9c8c76)c5)ccc34)ccc21 ; c1ccc(N(c2ccc(-c3ccc4ccccc4c3)cc2)c2cc(-c3cc4ccc5ccccc5c4c4c3ccc3ccccc34)c3c(c2)oc2ccccc23)cc1]

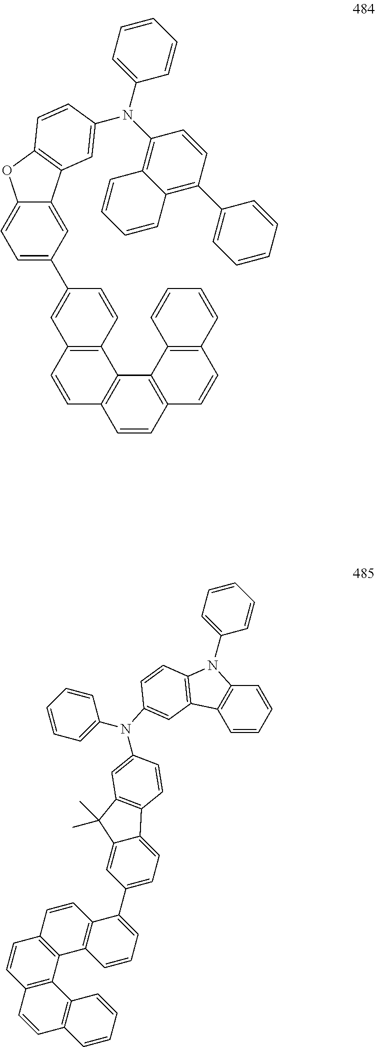 [SMILES: c1ccc(-c2ccc(N(c3ccccc3)c3ccc4oc5ccc(-c6ccc7c(ccc8ccc9ccc%10ccccc%10c9c87)c6)cc5c4c3)c3ccccc23)cc1 ; CC1(C)c2cc(-c3cccc4c3ccc3ccc5ccc6ccccc6c5c34)ccc2-c2ccc(N(c3ccccc3)c3ccc4c(c3)c3ccccc3n4-c3ccccc3)cc21]

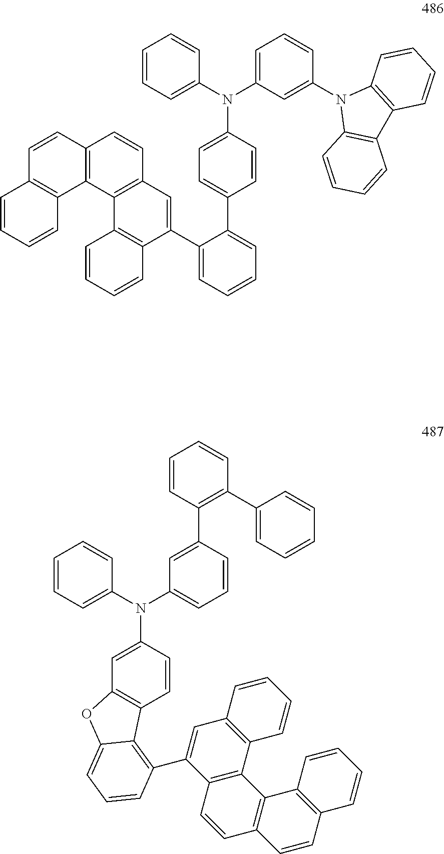 [SMILES: c1ccc(N(c2ccc(-c3ccccc3-c3cc4ccc5ccc6ccccc6c5c4c4ccccc34)cc2)c2cccc(-n3c4ccccc4c4ccccc43)c2)cc1 ; c1ccc(-c2ccccc2-c2cccc(N(c3ccccc3)c3ccc4c(c3)oc3cccc(-c5cc6ccccc6c6c5ccc5ccc7ccccc7c56)c34)c2)cc1]

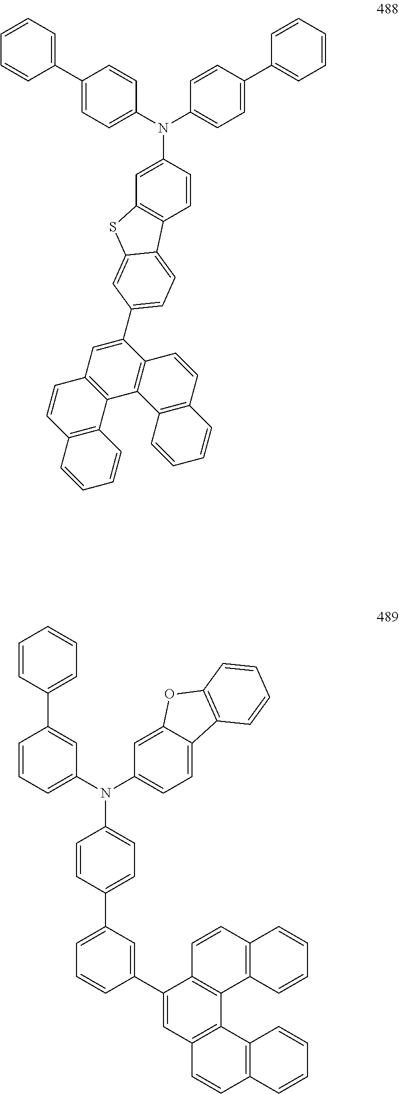 [SMILES: c1ccc(-c2ccc(N(c3ccc(-c4ccccc4)cc3)c3ccc4c(c3)sc3cc(-c5cc6ccc7ccccc7c6c6c5ccc5ccccc56)ccc34)cc2)cc1 ; c1ccc(-c2cccc(N(c3ccc(-c4cccc(-c5cc6ccc7ccccc7c6c6c5ccc5ccccc56)c4)cc3)c3ccc4c(c3)oc3ccccc34)c2)cc1]

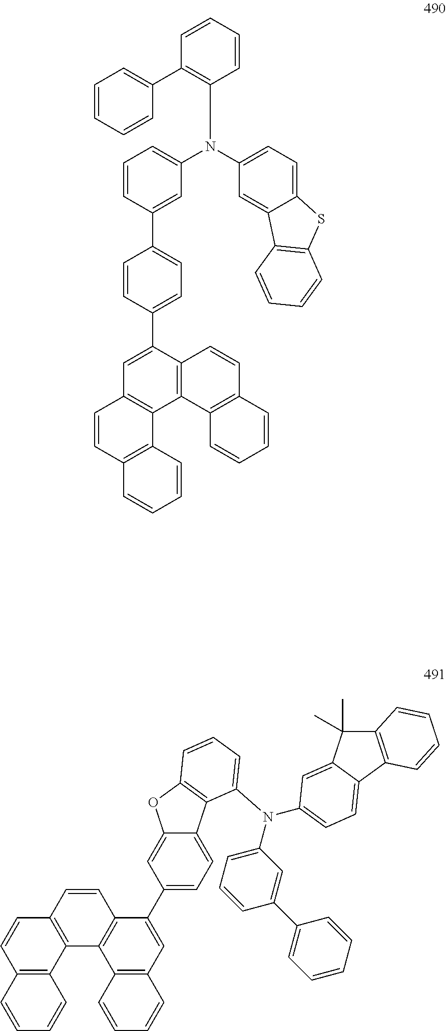 [SMILES: c1ccc(-c2ccccc2N(c2cccc(-c3ccc(-c4cc5ccc6ccccc6c5c5c4ccc4ccccc45)cc3)c2)c2ccc3sc4ccccc4c3c2)cc1 ; CC1(C)c2ccccc2-c2ccc(N(c3cccc(-c4ccccc4)c3)c3cccc4oc5cc(-c6cc7ccccc7c7c6ccc6ccc8ccccc8c67)ccc5c34)cc21]

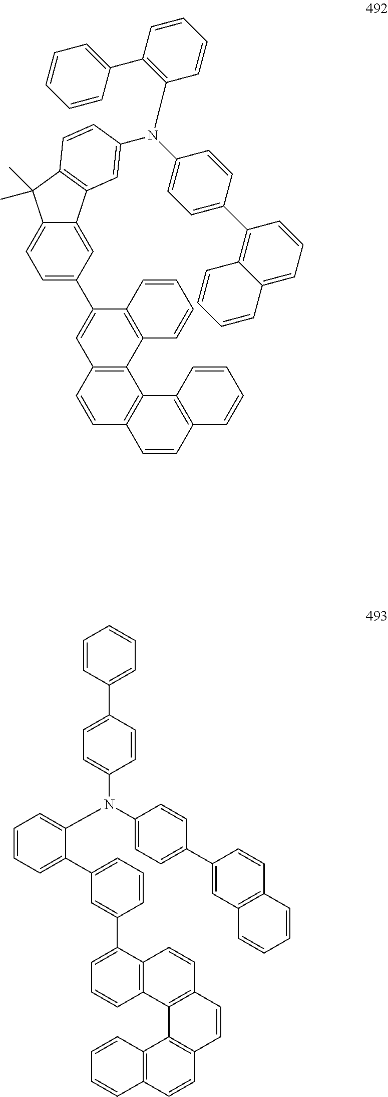 [SMILES: CC1(C)c2ccc(-c3cc4ccc5ccc6ccccc6c5c4c4ccccc34)cc2-c2cc(N(c3ccc(-c4cccc5ccccc45)cc3)c3ccccc3-c3ccccc3)ccc21 ; c1ccc(-c2ccc(N(c3ccc(-c4ccc5ccccc5c4)cc3)c3ccccc3-c3cccc(-c4cccc5c4ccc4ccc6ccc7ccccc7c6c45)c3)cc2)cc1]

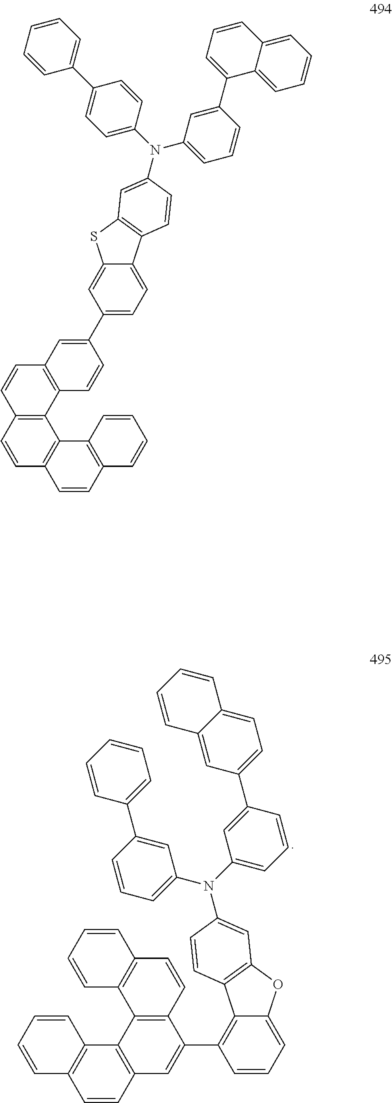 [SMILES: c1ccc(-c2ccc(N(c3cccc(-c4cccc5ccccc45)c3)c3ccc4c(c3)sc3cc(-c5ccc6c(ccc7ccc8ccc9ccccc9c8c76)c5)ccc34)cc2)cc1 ; c1ccc(-c2cccc(N(c3cccc(-c4ccc5ccccc5c4)c3)c3ccc4c(c3)oc3cccc(-c5cc6ccc7ccccc7c6c6c5ccc5ccccc56)c34)c2)cc1]

13. An organic electroluminescent device, comprising an anode and a cathode which are oppositely disposed, and a functional layer disposed between the anode and the cathode, wherein the functional layer comprises the arylamine compound according to claim 1.

14. The organic electroluminescent device according to claim 13, wherein the functional layer comprises an organic electroluminescent layer comprising the arylamine compound.

15. An electronic apparatus, comprising the organic electroluminescent device according to claim 13.

16. The arylamine compound according to claim 2, wherein the substituent(s) in Ar₁ and Ar₂ are each independently selected from deuterium, fluorine, cyano, haloalkyl with 1 to 4 carbon atoms, deuteroalkyl with 1 to 4 carbon atoms, alkyl with 1 to 4 carbon atoms, cycloalkyl with 5 to 10 carbon atoms, aryl with 6 to 12 carbon atoms, heteroaryl with 5 to 12 carbon atoms, and trialkylsilyl with 3 to 8 carbon atoms.

17. The arylamine compound according to claim 16, wherein any two adjacent substituents in Ar₁ and Ar₂ form a benzene ring or a fluorene ring.

18. The arylamine compound according to claim 5, wherein substituent(s) in L₁, L₂ and L₃ are each independently selected from deuterium, fluorine, cyano, haloalkyl with 1 to 4 carbon atoms, deuteroalkyl with 1 to 4 carbon atoms, alkyl with 1 to 4 carbon atoms, aryl with 6 to 10 carbon atoms, and trialkylsilyl with 3 to 8 carbon atoms.

19. The arylamine compound according to claim 6, wherein the substituent(s) in L₁, L₂ and L₃ are each independently selected from deuterium, fluorine, cyano, trimethylsilyl, trideuteromethyl, trifluoromethyl, methyl, ethyl, isopropyl, tert-butyl, phenyl or naphthyl.
